(19) United States
(12) Patent Application Publication
(10) Pub. No.: US 2024/0174637 A1
(43) Pub. Date: May 30, 2024

(54) DEGRADERS OF GRK2 AND USES THEREOF

(71) Applicants: Grazia PIIZZI, Cambridge, MA (US); Eugene L., Piatnitski CHEKLER, Cambridge, MA (US); Jonathan Barry HUROV, Bedford, MA (US); Alexandra LANTERMANN, Cambridge, MA (US); Kiley Marie COUTO, Cambridge, MA (US); Hua XU, Cambridge, MA (US); Sourav SARKAR, Cambridge, MA (US); Bruce Allen LEFKER, Nahant, MA (US); Ralph P. ROBINSON, Mystic, CT (US); Volodymyr KYSIL, San Diego, CA (US); Sonata Therapeutics, Inc., Cambridge, MA (US)

(72) Inventors: Grazia Piizzi, Brookline, MA (US); Eugene L. Piatnitski Chekler, Concord, MA (US); Jonathan Barry Hurov, Bedford, MA (US); Alexandra Lantermann, Cambridge, MA (US); Kiley Marie Couto, Quincy, MA (US); Hua Xu, Sharon, MA (US); Sourav Sarkar, North Chelmsford, MA (US); Bruce Allen Lefker, Nahant, MA (US); Ralph P. Robinson, Mystic, CT (US); Volodymyr Kysil, San Diego, CA (US)

(21) Appl. No.: 18/273,755

(22) PCT Filed: Jan. 21, 2022
(86) PCT No.: PCT/US2022/013295

§ 371 Date: Jul. 21, 2023

Related U.S. Application Data
(60) Provisional application No. 63/140,576, filed on Jan. 22, 2021.

(51) Int. Cl. C07D 401/14; C07D 405/14; C07D 417/14

(52) U.S. Cl. CPC C07D 401/14; C07D 405/14; C07D 417/14

(57) ABSTRACT

Provided herein are compounds (e.g., compounds of Formulae (A-I), (B-I), (C-I)), and pharmaceutically acceptable salts, stereoisomers, tautomers, isotopically labeled derivatives, solvates, hydrates, polymorphs, co-crystals, and prodrugs thereof, pharmaceutical compositions thereof, and kits comprising the same. The compounds provided herein are degraders of GRK2 proteins and are therefore useful for, e.g., treating and/or preventing diseases (e.g., cancer) in a subject, for inhibiting tumor growth in a subject, for inhibiting the activity of GRK2 and/or degrading a GRK2 protein in vitro or in vivo, etc. In certain embodiments, the compounds provided herein are selective for GRK2. Also provided herein are methods and synthetic intermediates useful in the preparation of compounds described herein.

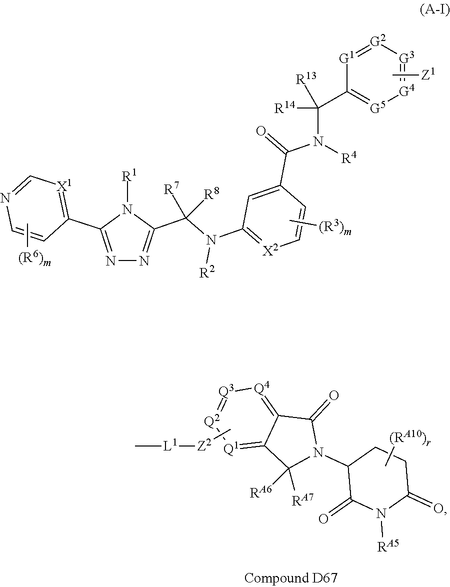

### RELATED APPLICATIONS

This application claims priority under 35 U.S.C. § 119(e) to U.S. Provisional Patent Application, U.S. Ser. No. 63/140,576, filed Jan. 22, 2021, the entire contents of which is incorporated herein by reference.

### BACKGROUND

G-protein-coupled receptor kinases (GRKs) participate in the processes of regulation of multiple G-protein-coupled receptors (GPCRs) of great physiological and pharmacological relevance. These proteins form a family of seven members that phosphorylate agonist-activated receptors in serine/threonine residues, promoting internalization, recycling and/or degradation processes of GPCRs.

GRK2, which is the most ubiquitous and best characterized isoform of the family of GRKs, has been found to regulate the activity of different GPCRs involved in diseases such as cancer, along with cytosolic proteins involved in proliferative and survival signaling pathways, as well as non-GPCRs membrane proteins with oncogenic potential. GRK2 protein levels and activity have also been reported to be enhanced in patients and/or in preclinical models of other diseases such as heart failure, cardiac hypertrophy, and hypertension.

Accordingly, there is a need to develop new compounds that decrease the level and/or activity of GRK2, including compounds that can inhibit and/or degrade GRK2 proteins.

### SUMMARY

Provided herein are degraders of GRK2 proteins, including compounds of any of the formulae herein, pharmaceutical compositions and kits comprising the same, and methods of using the same (e.g., for the treatment and/or prevention of diseases, e.g., cancer, in a subject). Also provided herein are methods of preparing the compounds and pharmaceutical compositions described herein.

In one aspect, provided herein are compounds of Formula (A-I):

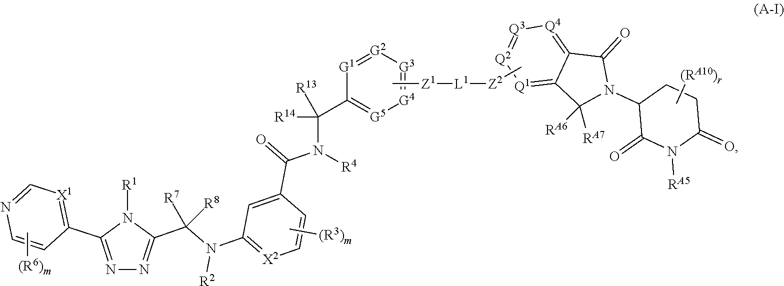

and pharmaceutically acceptable salts, stereoisomers, tautomers, isotopically labeled derivatives, solvates, hydrates, polymorphs, co-crystals, and prodrugs thereof, wherein the variables are as defined herein.

In certain embodiments, for example, a compound of Formula (A-I) is selected from the compounds recited in Table A (infra), and pharmaceutically acceptable salts, stereoisomers, tautomers, isotopically labeled derivatives, solvates, hydrates, polymorphs, co-crystals, and prodrugs thereof.

In another aspect, provided herein are compounds of Formula (B-I):

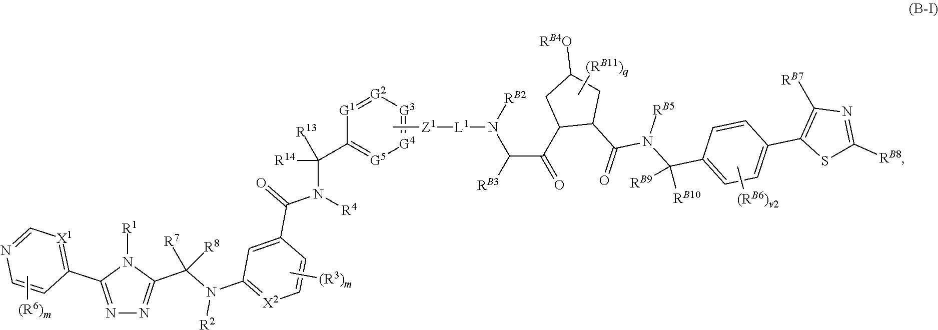

and pharmaceutically acceptable salts, stereoisomers, tautomers, isotopically labeled derivatives, solvates, hydrates, polymorphs, co-crystals, and prodrugs thereof, wherein the variables are as defined herein.

In certain embodiments, for example, a compound of Formula (B-I) is selected from the compounds recited in Table B (infra), and pharmaceutically acceptable salts, stereoisomers, tautomers, isotopically labeled derivatives, solvates, hydrates, polymorphs, co-crystals, and prodrugs thereof.

In another aspect, provided herein are compounds of Formula (C-I):

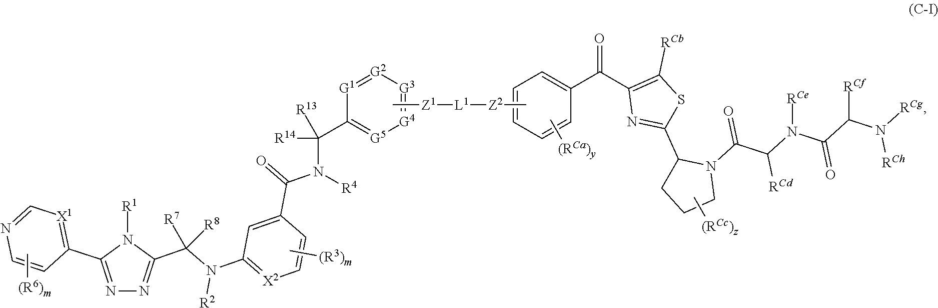

and pharmaceutically acceptable salts, stereoisomers, tautomers, isotopically labeled derivatives, solvates, hydrates, polymorphs, co-crystals, and prodrugs thereof, wherein the variables are as defined herein.

In certain embodiments, for example, a compound of Formula (C-I) is selected from the compounds recited in Table C (infra), and pharmaceutically acceptable salts, stereoisomers, tautomers, isotopically labeled derivatives, solvates, hydrates, polymorphs, co-crystals, and prodrugs thereof.

The compounds provided herein are degraders of GRK family member proteins (e.g., GRK2) and are therefore useful for treating and/or preventing diseases (e.g., cancer) in a subject. In certain embodiments, the compounds provided herein are GRK2 degraders. In certain embodiments, the compounds provided herein are selective GRK2 degraders, i.e., selective for GRK2 over other kinases (e.g., over other GRK family member proteins).

In another aspect, provided herein are pharmaceutical compositions comprising a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, and one or more pharmaceutically acceptable carriers or excipients. In certain embodiments, a pharmaceutical composition provided herein comprises a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof. The pharmaceutical compositions described herein are useful for treating and/or preventing diseases (e.g., cancer) in a subject. The pharmaceutical compositions provided herein may further comprise one or more additional therapeutic agents (e.g., anti-cancer agents).

In other aspects, provided herein are methods and uses of the compounds and pharmaceutical compositions provided herein, including the following:
(a) Methods of treating and/or preventing a proliferative disease (e.g., cancer) in a subject comprising administering to the subject a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the proliferative disease is cancer. In certain embodiments, the proliferative disease is a cancer related to the activity of a GRK family member protein (e.g., GRK2) in a subject or cell.
(b) Methods of inhibiting tumor growth in a subject comprising administering to the subject a therapeutically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof.
(c) Methods of treating and/or preventing a cardiovascular disease in a subject comprising administering to the subject a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the cardiovascular disease is heart failure, cardiac hypertrophy, or hypertension. In certain embodiments, the cardiovascular disease is related to the activity of a GRK family member protein (e.g., GRK2) in a subject or cell.
(d) Methods of treating opioid addiction in a subject comprising administering to the subject a therapeutically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof.
(e) Methods of treating and/or preventing a GRK2-related disease in a subject comprising administering to the subject a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the GRK2-related disease is related to increased activity of GRK2 in a subject.
(f) Methods of degrading a GRK2 protein in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the degrading occurs in vivo (i.e., in a subject). In certain embodiments, the degrading occurs in vitro (e.g., in a cell line or biological sample). In certain embodiments, the degradation is selective GRK2 degradation.
(g) Methods of inhibiting the activity of GRK2 in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the inhibiting occurs in vivo (i.e., in a subject). In certain embodiments, the inhibiting occurs in vitro (e.g., in a cell line or biological sample). In certain embodiments, the inhibition is selective GRK2 inhibition.

In another aspect, provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in any of the methods provided herein.

In yet another aspect, provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the preparation of medicaments (e.g., for treating and/or preventing a disease, e.g., cancer, in a subject).

In another aspect, provided herein are kits comprising a compound of (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. The kits described herein may include a single dose or multiple doses of the compound or pharmaceutical composition thereof. The kits described herein are useful in any method or use provided herein, and optionally further comprise instructions for using the kit (e.g., instructions for using the compound or composition included in the kit).

Also provided herein are methods of preparing compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof. Synthetic intermediates useful in the preparation of the compounds are also provided herein and are considered to be part of the disclosure.

The details of certain embodiments of the disclosure are set forth in the Detailed Description of Certain Embodiments, as described below. Other features, objects, and advantages of the disclosure will be apparent from the Definitions, Examples, Figures, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms (“isotopically labeled derivatives”). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C_{3- 5}, C₃₋₄, C₄₋₆, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“C₁₋₂₀ heteroalkyl”). In certain embodiments, a heteroalkyl group refers to a saturated group having from 1 to 12 carbon atoms and 1 or more heteroatoms within the parent chain (“C₁₋₁₂ heteroalkyl”). In some embodiments, a heteroalkyl group is a saturated group having 1 to 11 carbon atoms and 1 or more heteroatoms within the parent chain (“C₁₋₁₁ heteroalkyl”). In some embodiments, a heteroalkyl group is a saturated group having 1 to 10 carbon atoms and 1 or more heteroatoms within the parent chain (“C₁₋₁₀ heteroalkyl”). In some embodiments, a heteroalkyl group is a saturated group having 1 to 9 carbon atoms and 1 or more heteroatoms within the parent chain (“C₁₋₉ heteroalkyl”). In some embodiments, a heteroalkyl group is a saturated group having 1 to 8 carbon atoms and 1 or more heteroatoms within the parent chain (“C₁₋₈ heteroalkyl”). In some embodiments, a heteroalkyl group is a saturated group having 1 to 7 carbon atoms and 1 or more heteroatoms within the parent chain (“C₁₋₇ heteroalkyl”). In some embodiments, a heteroalkyl group is a saturated group having 1 to 6 carbon atoms and 1 or more heteroatoms within the parent chain (“C₁₋₆ heteroalkyl”). In some embodiments, a heteroalkyl group is a saturated group having 1 to 5 carbon atoms and 1 or 2 heteroatoms within the parent chain (“C₁₋₅ heteroalkyl”). In some embodiments, a heteroalkyl group is a saturated group having 1 to 4 carbon atoms and 1 or 2 heteroatoms within the parent chain (“C₁₋₄ heteroalkyl”). In some embodiments, a heteroalkyl group is a saturated group having 1 to 3 carbon atoms and 1 heteroatom within the parent chain (“C₁₋₃ heteroalkyl”). In some embodiments, a heteroalkyl group is a saturated group having 1 to 2 carbon atoms and 1 heteroatom within the parent chain (“C₁₋₂ heteroalkyl”). In some embodiments, a heteroalkyl group is a saturated group having 1 carbon atom and 1 heteroatom (“C₁ heteroalkyl”). In some embodiments, a heteroalkyl group is a saturated group having 2 to 6 carbon atoms and 1 or 2 heteroatoms within the parent chain (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In an alkenyl group, a C═C double bond for which the stereochemistry is not specified (e.g., —CH═CHCH₃ or

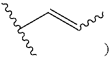

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“C₁₋₂₀ heteroalkenyl”). In certain embodiments, a heteroalkenyl group refers to a group having from 1 to 12 carbon atoms, at least one double bond, and 1 or more heteroatoms within the parent chain (“C₁₋₁₂ heteroalkenyl”). In certain embodiments, a heteroalkenyl group refers to a group having from 1 to 11 carbon atoms, at least one double bond, and 1 or more heteroatoms within the parent chain (“C₁₋₁₁ heteroalkenyl”). In certain embodiments, a heteroalkenyl group refers to a group having from 1 to 10 carbon atoms, at least one double bond, and 1 or more heteroatoms within the parent chain (“C₁₋₁₀ heteroalkenyl”). In some embodiments, a heteroalkenyl group has 1 to 9 carbon atoms at least one double bond, and 1 or more heteroatoms within the parent chain (“C₁₋₉ heteroalkenyl”). In some embodiments, a heteroalkenyl group has 1 to 8 carbon atoms, at least one double bond, and 1 or more heteroatoms within the parent chain (“C₁₋₈ heteroalkenyl”). In some embodiments, a heteroalkenyl group has 1 to 7 carbon atoms, at least one double bond, and 1 or more heteroatoms within the parent chain (“C₁₋₇ heteroalkenyl”). In some embodiments, a heteroalkenyl group has 1 to 6 carbon atoms, at least one double bond, and 1 or more heteroatoms within the parent chain (“C₁₋₆ heteroalkenyl”). In some embodiments, a heteroalkenyl group has 1 to 5 carbon atoms, at least one double bond, and 1 or 2 heteroatoms within the parent chain (“C₁₋₅ heteroalkenyl”). In some embodiments, a heteroalkenyl group has 1 to 4 carbon atoms, at least one double bond, and 1 or 2 heteroatoms within the parent chain (“C₁₋₄ heteroalkenyl”). In some embodiments, a heteroalkenyl group has 1 to 3 carbon atoms, at least one double bond, and 1 heteroatom within the parent chain (“C₁₋₃ heteroalkenyl”). In some embodiments, a heteroalkenyl group has 1 to 2 carbon atoms, at least one double bond, and 1 heteroatom within the parent chain (“C₁₋₂ heteroalkenyl”). In some embodiments, a heteroalkenyl group has 1 to 6 carbon atoms, at least one double bond, and 1 or 2 heteroatoms within the parent chain (“C₁₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“C₁₋₂₀ heteroalkynyl”). In certain embodiments, a heteroalkynyl group refers to a group having from 1 to 10 carbon atoms, at least one triple bond, and 1 or more heteroatoms within the parent chain (“C₁₋₁₀ heteroalkynyl”). In some embodiments, a heteroalkynyl group has 1 to 9 carbon atoms, at least one triple bond, and 1 or more heteroatoms within the parent chain (“C₁₋₉ heteroalkynyl”). In some embodiments, a heteroalkynyl group has 1 to 8 carbon atoms, at least one triple bond, and 1 or more heteroatoms within the parent chain (“C₁₋₈ heteroalkynyl”). In some embodiments, a heteroalkynyl group has 1 to 7 carbon atoms, at least one triple bond, and 1 or more heteroatoms within the parent chain (“C₁₋₇ heteroalkynyl”). In some embodiments, a heteroalkynyl group has 1 to 6 carbon atoms, at least one triple bond, and 1 or more heteroatoms within the parent chain (“C₁₋₆ heteroalkynyl”). In some embodiments, a heteroalkynyl group has 1 to 5 carbon atoms, at least one triple bond, and 1 or 2 heteroatoms within the parent chain (“C₁₋₅ heteroalkynyl”). In some embodiments, a heteroalkynyl group has 1 to 4 carbon atoms, at least one triple bond, and 1 or 2 heteroatoms within the parent chain (“C₁₋₄ heteroalkynyl”). In some embodiments, a heteroalkynyl group has 1 to 3 carbon atoms, at least one triple bond, and 1 heteroatom within the parent chain (“C₁₋₃ heteroalkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃1₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl is substituted or unsubstituted, 3- to 8-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydro-pyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

Exemplary tricyclic heteroaryl groups include phenanthridinyl, dibenzofuranyl, carbazolyl, acridinyl, phenothiazinyl, phenoxazinyl, and phenazinyl.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “halo” or “halogen” refers to fluorine (fluoro, —F), chlorine (chloro, —Cl), bromine (bromo, —Br), or iodine (iodo, —I).

The term “silyl” refers to the group —Si(R^{aa})₃, wherein R^{aa} is as defined herein.

The term “unsaturated bond” refers to a double or triple bond. The term “unsaturated” or “partially unsaturated” refers to a moiety that includes at least one double or triple bond. The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen, oxygen, and sulfur may have hydrogen substituents and/or any suitable substituent as described herein which satisfy the valencies of the heteroatoms and results in the formation of a stable moiety. The embodiments described herein are not limited in any manner by the exemplary substituents described herein.

Exemplary substituents (e.g., carbon atom substituents) include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, C₁₋₂₀ heteroalkyl, C₁₋₂₀ heteroalkenyl, C₁₋₂₀ heteroalkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R—, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, C₁₋₂₀ heteroalkyl, C₁₋₂₀ heteroalkenyl, C₁₋₂₀ heteroalkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{a}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, C₁₋₂₀ heteroalkyl, C₁₋₂₀ heteroalkenyl, C₁₋₂₀ heteroalkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, C₁₋₂₀ heteroalkyl, C₁₋₂₀ heteroalkenyl, C₁₋₂₀ heteroalkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, C₁₋₁₀ heteroalkyl, C₁₋₁₀ heteroalkenyl, C₁₋₁₀ heteroalkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, C₁₋₁₀ heteroalkyl, C₁₋₁₀ heteroalkenyl, C₁₋₁₀ heteroalkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, C₁₋₁₀ heteroalkyl, C₁₋₁₀ heteroalkenyl, C₁₋₁₀ heteroalkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₄ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, C₁₋₁₀ heteroalkyl, C₁₋₁₀ heteroalkenyl, C₁₋₁₀ heteroalkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and each X⁻ is a counterion.

In certain embodiments, the molecular weight of a substituent (e.g., carbon atom substituent) is lower than 250, lower than 200, lower than 150, lower than 100, or lower than 50 g/mol.

In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, or —NO₂. In certain embodiments, each carbon atom substituent is independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that includes the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached).

In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that includes the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds). In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{a}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv). In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, and Claims. The embodiments provided herein are not limited in any manner by the above exemplary listing of substituents.

### Other Definitions

The following definitions are more general terms used throughout the present application.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

“Stereoisomers” that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 20 of, e.g., between 20 and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 20 of between 20 and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, e.g., Bundgard, H., Design of Prodrugs, pp. 7-9, 21-24, Elsevier, Amsterdam 1985). Prodrugs include acid derivatives such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester-type prodrugs such as (acyloxy)alkyl esters or ((alkoxycarbonyl)oxy)alkylesters. Aliphatic or aromatic (e.g., alkyl, alkenyl, alkynyl, aryl, or arylalkyl) esters of the compounds described herein may be preferred.

Throughout the present disclosure, references to “the compound” and “a compound” provided herein are intended to encompass the compound or group of compounds, and also pharmaceutically acceptable salts, stereoisomers, tautomers, isotopically labeled derivatives, solvates, hydrates, polymorphs, co-crystals, and prodrugs thereof as described herein.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting GRK2 activity in a subject. In certain embodiments, a therapeutically effective amount is an amount sufficient for degrading a GRK2 protein in a subject. In certain embodiments, a therapeutically effective amount is an amount sufficient for treating a proliferative disease (e.g., cancer) in a subject. In certain embodiments, a therapeutically effective amount is an amount sufficient for treating a cardiovascular disease in a subject. In certain embodiments, a therapeutically effective amount is an amount sufficient for treating a GRK2-related disease in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting GRK2 activity in a subject. In certain embodiments, a prophylactically effective amount is an amount sufficient for degrading a GRK2 protein in a subject. In certain embodiments, a prophylactically effective amount is an amount sufficient for preventing a proliferative disease (e.g., cancer) in a subject. In certain embodiments, a prophylactically effective amount is an amount sufficient for preventing a cardiovascular disease in a subject. In certain embodiments, a prophylactically effective amount is an amount sufficient for preventing a GRK2-related disease in a subject.

As used herein, the term “GRK2” refers to G-protein-coupled receptor kinase 2 and belongs to the G-protein-coupled receptor kinase subfamily of the Ser/Thr protein kinases. GRK2 is encoded by the ADRBK1 gene, the nucleic acid sequence of which is set forth in SEQ ID NO: 1, below:

(SEQ ID NO: 1)
ATGGCGGACCTGGAGGCGGTGCTGGCCGACGTGAGCTACCTGATGGCCATGGAGAAGAGCAAGGCCACGC
CGGCCGCGCGCGCCAGCAAGAAGATCCTGCTGCCCGAGCCCAGCATCCGCAGTGTCATGCAGAAGTACCT
GGAGGACCGGGGCGAGGTGACCTTTGAGAAGATCTTTTCCCAGAAGCTGGGGTACCTGCTCTTCCGAGAC
TTCTGCCTGAACCACCTGGAGGAGGCCAGGCCCTTGGTGGAATTCTATGAGGAGATCAAGAAGTACGAGA
AGCTGGAGACGGAGGAGGAGCGTGTGGCCCGCAGCCGGGAGATCTTCGACTCATACATCATGAAGGAGCT
GCTGGCCTGCTCGCATCCCTTCTCGAAGAGTGCCACTGAGCATGTCCAAGGCCACCTGGGGAAGAAGCAG
GTGCCTCCGGATCTCTTCCAGCCATACATCGAAGAGATTTGTCAAAACCTCCGAGGGGACGTGTTCCAGA
AATTCATTGAGAGCGATAAGTTCACACGGTTTTGCCAGTGGAAGAATGTGGAGCTCAACATCCACCTGAC
CATGAATGACTTCAGCGTGCATCGCATCATTGGGCGCGGGGGCTTTGGCGAGGTCTATGGGTGCCGGAAG
GCTGACACAGGCAAGATGTACGCCATGAAGTGCCTGGACAAAAAGCGCATCAAGATGAAGCAGGGGGAGA
CCCTGGCCCTGAACGAGCGCATCATGCTCTCGCTCGTCAGCACTGGGGACTGCCCATTCATTGTCTGCAT
GTCATACGCGTTCCACACGCCAGACAAGCTCAGCTTCATCCTGGACCTCATGAACGGTGGGGACCTGCAC
TACCACCTCTCCCAGCACGGGGTCTTCTCAGAGGCTGACATGCGCTTCTATGCGGCCGAGATCATCCTGG
GCCTGGAGCACATGCACAACCGCTTCGTGGTCTACCGGGACCTGAAGCCAGCCAACATCCTTCTGGACGA
GCATGGCCACGTGCGGATCTCGGACCTGGGCCTGGCCTGTGACTTCTCCAAGAAGAAGCCCCATGCCAGC
GTGGGCACCCACGGGTACATGGCTCCGGAGGTCCTGCAGAAGGGCGTGGCCTACGACAGCAGTGCCGACT
GGTTCTCTCTGGGGTGCATGCTCTTCAAGTTGCTGCGGGGGCACAGCCCCTTCCGGCAGCACAAGACCAA
AGACAAGCATGAGATCGACCGCATGACGCTGACGATGGCCGTGGAGCTGCCCGACTCCTTCTCCCCTGAA
CTACGCTCCCTGCIGGAGGGGTTGCTGCAGAGGGATGTCAACCGGAGATTGGGCTGCCTGGGCCGAGGGG
CTCAGGAGGIGAAAGAGAGCCCCTTTTTCCGCTCCCTGGACTGGCAGATGGTCTTCTTGCAGAAGTACCC
TCCCCCGCTGATCCCCCCACGAGGGGAGGTGAACGCGGCCGACGCCTTCGACATTGGCTCCTTCGATGAG
GAGGACACAAAAGGAATCAAGTTACTGGACAGTGATCAGGAGCTCTACCGCAACTTCCCCCTCACCATCT
CGGAGCGGTGGCAGCAGGAGGTGGCAGAGACTGTCTTCGACACCATCAACGCTGAGACAGACCGGCTGGA
GGCTCGCAAGAAAGCCAAGAACAAGCAGCTGGGCCATGAGGAAGACTACGCCCTGGGCAAGGACTGCATC
ATGCATGGCTACATGTCCAAGATGGGCAACCCCTTCCTGACCCAGTGGCAGCGGCGGTACTTCTACCTGT
TCCCCAACCGCCTCGAGTGGCGGGGCGAGGGCGAGGCCCCGCAGAGCCTGCTGACCATGGAGGAGATCCA
GTCGGTGGAGGAGACGCAGATCAAGGAGCGCAAGTGCCTGCTCCTCAAGATCCGCGGTGGGAAACAGTTC
ATTTTGCAGTGCGATAGCGACCCTGAGCTGGIGCAGTGGAAGAAGGAGCTGCGCGACGCCTACCGCGAGG
CCCAGCAGCTGGTGCAGCGGGTGCCCAAGATGAAGAACAAGCCGCGCTCGCCCGTGGTGGAGCTGAGCAA
GGTGCCGCTGGTCCAGCGCGGCAGTGCCAACGGCCTCTGA

The term “GRK2” also refers to natural variants of the wild-type GRK2 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type GRK2, which is set forth in SEQ ID NO: 2, below:

(SEQ ID NO: 2)
MADLEAVLADVSYLMAMEKSKATPAARASKKILLPEPSIRSVMQKYLEDR
GEVTFEKIFSQKLGYLLERDFCLNHLEEARPLVEFYEEIKKYEKLETEEE
RVARSREIFDSYIMKELLACSHPFSKSATEHVQGHLGKKQVPPDLFQPYI
EEICQNLRGDVFQKFIESDKFTRFCQWKNVELNIHLIMNDFSVHRIIGRG
GFGEVYGCRKADTGKMYAMKCLDKKRIKMKQGETLALNERIMLSLVSTGD
CPFIVCMSYAFHTPDKLSFILDLMNGGDLHYHLSQHGVFSEADMRFYAAE
IILGLEHMHNRFVVYRDLKPANILLDEHGHVRISDLGLACDFSKKKPHAS
VGTHGYMAPEVLQKGVAYDSSADWFSLGCMLFKLLRGHSPFRQHKTKDKH
EIDRMTLTMAVELPDSFSPELRSLLEGLLQRDVNRRLGCLGRGAQEVKES
PFFRSLDWQMVFLQKYPPPLIPPRGEVNAADAFDIGSFDEEDTKGIKLLD
SDQELYRNFPLTISERWQQEVAETVEDTINAETDRLEARKKAKNKQLGHE
EDYALGKDCIMHGYMSKMGNPFLTQWORRYFYLFPNRLEWRGEGEAPQSL
LTMEEIQSVEETQIKERKCLLLKIRGGKQFILQCDSDPELVQWKKELRDA
YREAQQLVQRVPKMKNKPRSPVVELSKVPLVQRGSANGL

As used herein, the term “GRK2-related disease” refers to a diseases or condition that is associated with cells that express or overexpress GRK2 (e.g., cancer cells that express or overexpress GRK2 compared to a reference). In certain embodiments, a GRK2-related disease is a disease or condition associated with aberrant (e.g., increased) activity of GRK2 in a subject. GRK2-related disorders can be identified by assessing a cell or a biopsy of a tissue sample for GRK2 expression and comparing it to GRK2 expression in a reference cell or tissue sample.

As used herein, “degrade” or “degrading” in the context of protein, for example, in the context of GRK2, refers to metabolizing or breaking down said protein. In some embodiments, degrading a protein (e.g., GRK2) leads to a reduction of the level of activity of the protein (e.g., GRK2 activity) or a downstream effect, e.g., relative to a baseline or control level of enzyme activity. As used herein, the term “degrader” refers to a small molecule compound including a degradation moiety, wherein the compound interacts with a protein (e.g., GRK2) in a way that results in partial or complete degradation of the protein in a cell or subject. As used herein, the term “degradation moiety” refers to a moiety whose binding results in partial or complete degradation of a protein (e.g., GRK2). In one example, the moiety binds to a protease or a ubiquitin ligase that metabolizes the protein (e.g., GRK2).

As used herein, the term “GRK3” refers to G-protein-coupled receptor kinase 3 and belongs to the G-protein-coupled receptor kinase subfamily of the Ser/Thr protein kinases. Human GRK3 is encoded by the ADRBK2 gene. The term “GRK3” also refers to natural variants of the wild-type GRK3 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type human GRK3, which is set forth in SEQ ID NO: 3, below:

(SEQ ID NO: 3)
MADLEAVLADVSYLMAMEKSKATPAARASKRIVLPEPSIRSVMQKYLAER
NEITFDKIFNQKIGFLLFKDFCLNEINEAVPQVKFYEEIKEYEKLDNEED
RLCRSRQIYDAYIMKELLSCSHPFSKQAVEHVQSHLSKKQVISTLFQPYI
EEICESLRGDIFQKFMESDKFTRFCQWKNVELNIHLIMNEFSVHRIIGRG
GFGEVYGCRKADTGKMYAMKCLDKKRIKMKQGETLALNERIMLSLVSTGD
CPFIVCMTYAFHTPDKLCFILDLMNGGDLHYHLSQHGVFSEKEMRFYATE
IILGLEHMHNRFVVYRDLKPANILLDEHGHARISDLGLACDFSKKKPHAS
VGTHGYMAPEVLQKGTAYDSSADWFSLGCMLFKLLRGHSPFRQHKTKDKH
EIDRMILTVNVELPDTFSPELKSLLEGLLQRDVSKRLGCHGGGSQEVKEH
SFFKGVDWQHVYLQKYPPPLIPPRGEVNAADAFDIGSFDEEDIKGIKLLD
CDQELYKNFPLVISERWQQEVTETVYEAVNADIDKIEARKRAKNKQLGHE
EDYALGKDCIMHGYMLKLGNPFLIQWORRYFYLFPNRLEWRGEGESRQNL
LIMEQILSVEETQIKDKKCILFRIKGGKQFVLQCESDPEFVQWKKELNET
FKEAQRLLRRAPKFLNKPRSGTVELPKPSLCHRNSNGL

As used herein, the term “GRK3-related disease” refers to a diseases or condition that is associated with cells that express or overexpress GRK3 (e.g., cancer cells that express or overexpress GRK3 compared to a reference). In certain embodiments, a GRK3-related disease is a disease or condition associated with aberrant (e.g., increased) activity of GRK3 in a subject. GRK3-related disorders can be identified by assessing a cell or a biopsy of a tissue sample for GRK3 expression and comparing it to GRK3 expression in a reference cell or tissue sample.

As used herein the term “inhibit,” “inhibition,” or “inhibiting” in the context of enzymes, for example, in the context of GRK2, refers to a reduction in the activity of the enzyme or a downstream effect. In some embodiments, the term refers to a reduction of the level of enzyme activity (e.g., GRK2 activity) to a level that is statistically significantly lower than an initial level, which may, for example, be a baseline or control level of enzyme activity. In some embodiments, the term refers to a reduction of the level of enzyme activity (e.g., GRK2 activity) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

As used herein, “level” means a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, g/mL, ng/mL) or percentage relative to total protein or mRNA in a sample.

In certain embodiments, a compound described herein is a “selective” degrader and/or inhibitor that degrades and/or inhibits one or more enzymes to a greater extent than over other enzymes. In certain embodiments, the compounds provided herein are selective GRK2 degraders, i.e., that selectively degrade GRK2 over other proteins (e.g., over other protein kinases, e.g., over other GRK family member proteins). In certain embodiments, the selectivity is at least 2-fold, at least 3-fold, at least 5-fold, at least 10-fold, at least 30-fold, at least 50-fold, at least 100-fold, at least 300-fold, at least 500-fold, at least 1,000-fold, at least 3,000-fold, at least 5,000-fold, at least 10,000-fold, at least 30,000-fold, at least 50,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

For example, the selectivity of a compound described herein in inhibiting the activity of GRK2 over a different protein (e.g., a different GRK2 family member protein) may be measured by the quotient of the IC₅₀ value of the compound in inhibiting the activity of the different protein over the IC₅₀ value of the compound in inhibiting the activity of GRK2. The selectivity of a compound described herein for GRK2 over a different protein (e.g., a different GRK family member protein) may also be measured by the quotient of the K_{d} value of an adduct of the compound and the different protein over the K_{d} value of an adduct of the compound and GRK2.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

### BRIEF DESCRIPTION OF THE DRAWINGS

The accompanying drawings, which are incorporated in and constitute a part of this specification, illustrate several embodiments of the disclosure and together with the description, provide non-limiting examples of the disclosure.

FIGS. 1A-ID. Dose dependent GRK2 degradation of D67 (A), D73 (B), D64 (C), and D66 (D) after 24-hour treatment in PAXF1657 cells.

### DETAILED DESCRIPTION OF CERTAIN EMBODIMENTS

Provided herein are degraders of GRK2 proteins, including compounds of any of the formulae herein (e.g., Formulae (A-I), (B-I), and (C-I)), pharmaceutical compositions and kits comprising the same, and methods of using the same (e.g., for the treatment and/or prevention of diseases, e.g., cancer, in a subject). In certain embodiments, the compounds provided herein comprise degradation moieties that can aid in the degradation of GRK2 proteins in a cell and/or in a subject. Also provided herein are methods of preparing the compounds and pharmaceutical compositions described herein.

### Compounds

Provided herein are compounds of Formula (A-I):

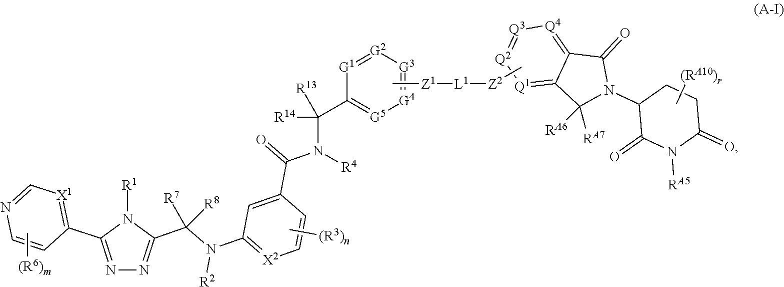

and pharmaceutically acceptable salts, stereoisomers, tautomers, isotopically labeled derivatives, solvates, hydrates, polymorphs, co-crystals, and prodrugs thereof, wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, or optionally substituted 5-10 membered heteroarylene;
Z¹ is a bond, —O—, —NR^{N}—, —S—, —C(═O)—, or optionally substituted —CH₂—;
Z² is a bond, —O—, —NR^{A11}—, —S—, —C(═O)—, or optionally substituted —CH₂—;
G¹ is CR¹⁵ or N;
G², G³, G⁴, and G⁵ are each independently CR¹⁶, CH, or N;
Q₁, Q², Q³, and Q⁴ are each independently CR^{A1}, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{A5}, and R^{A11} are each independently hydrogen, optionally substituted C₁₋₆ alkyl, optionally substituted C₁₋₆ acyl, or a nitrogen protecting group;
each instance of R³, R⁶, R⁹, R¹⁵, R¹⁶, and R^{A1} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R⁸ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{A6} and R^{A7} are independently hydrogen or optionally substituted C₁₋₆ alkyl, or optionally R^{A6} and R^{A7} are joined together with the intervening atoms to form optionally substituted C₃₋₈ carbocyclyl or 3-8 membered heterocyclyl, or optionally R^{A6} and R^{A7} are taken together to form ═O;
each instance of R^{A10} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
m and n are each independently is 0, 1, 2, or 3; and
r is 0, 1, 2, 3, 4, or 5.

In certain embodiments, a compound of Formula (A-I) is of Formula (A-II):

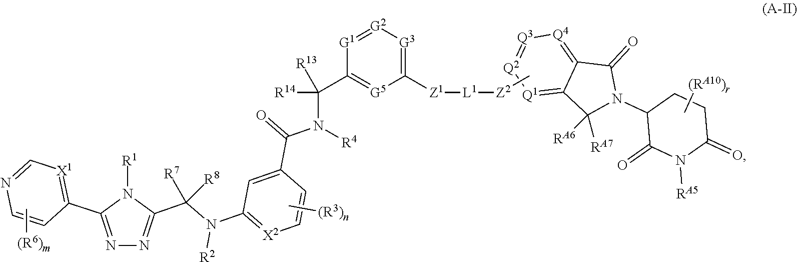

or a pharmaceutically acceptable salt, stereoisomer, tautomer, isotopically labeled derivative, solvate, hydrate, polymorph, co-crystal, or prodrug thereof.

In certain embodiments, a compound of Formula (A-I) is of Formula (A-III):

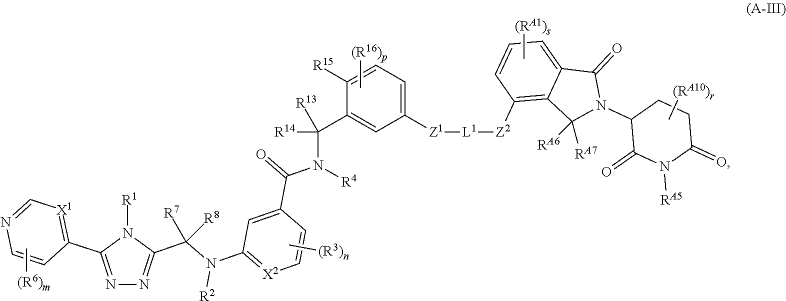

or a pharmaceutically acceptable salt, stereoisomer, tautomer, isotopically labeled derivative, solvate, hydrate, polymorph, co-crystal, or prodrug thereof, wherein p and s are each independently 0, 1, 2, or 3.

In certain embodiments, a compound of Formula (A-I) is of Formula (A-IV):

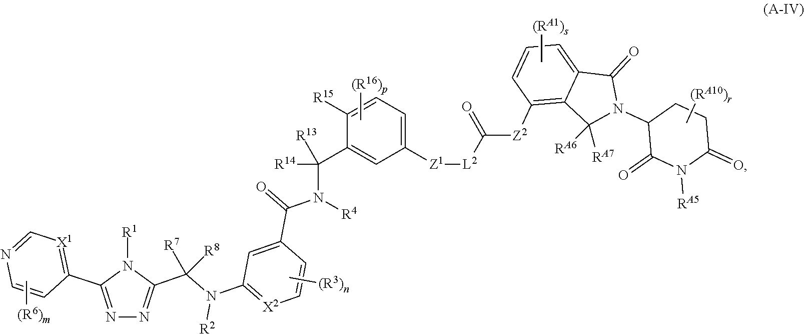

or a pharmaceutically acceptable salt, stereoisomer, tautomer, isotopically labeled derivative, solvate, hydrate, polymorph, co-crystal, or prodrug thereof, wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

In certain embodiments, a compound of Formula (A-I) is of Formula (A-V):

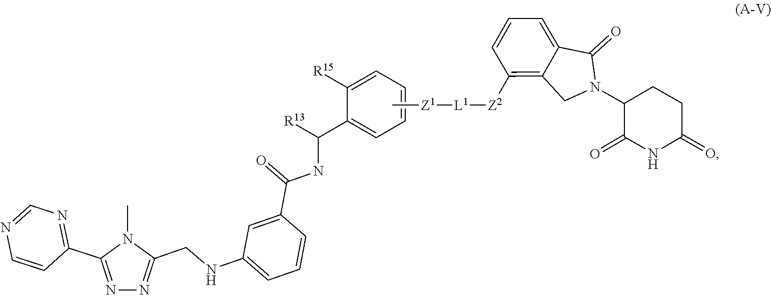

or a pharmaceutically acceptable salt, stereoisomer, tautomer, isotopically labeled derivative, solvate, hydrate, polymorph, co-crystal, or prodrug thereof.

In certain embodiments, a compound of Formula (A-I) is of Formula (A-VI):

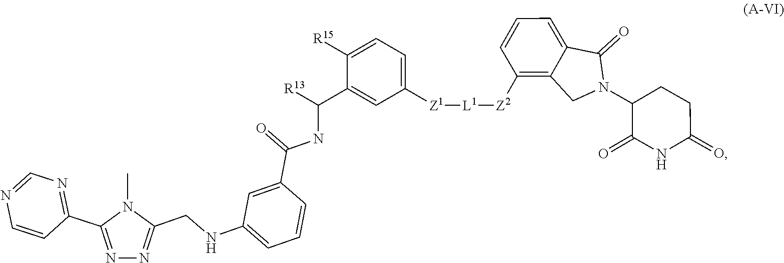

or a pharmaceutically acceptable salt, stereoisomer, tautomer, isotopically labeled derivative, solvate, hydrate, polymorph, co-crystal, or prodrug thereof.

In certain embodiments, a compound of Formula (A-I) is of Formula (A-VII):

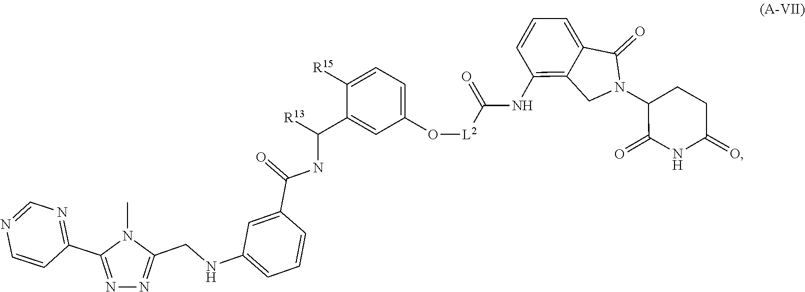

or a pharmaceutically acceptable salt, stereoisomer, tautomer, isotopically labeled derivative, solvate, hydrate, polymorph, co-crystal, or prodrug thereof.

In certain embodiments, for example, a compound of Formula (A-I) is selected from the compounds recited in Table A (infra), and pharmaceutically acceptable salts, stereoisomers, tautomers, isotopically labeled derivatives, solvates, hydrates, polymorphs, co-crystals, and prodrugs thereof.

In the various aspects and embodiments disclosed herein, express reference to a compound of Formula (A-I) is understood to alternatively refer to a compound of any disclosed subgenus or species thereof, for example, Formulae (A-II)-(A-VII), or to a compound of Table A (infra).

TABLE A
Examples of Compounds of Formula (A-I)

| | Com- |
|---|---|
| Structure | pound |
| 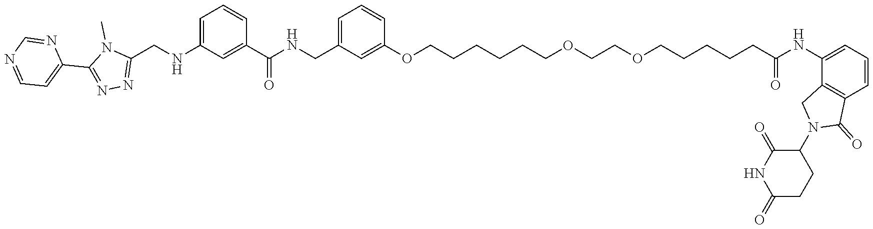 [SMILES: Cn1c(CNc2cccc(C(=O)NCc3cccc(OCCCCCCOCCOCCCCCC(=O)Nc4cccc5c4CN(C4CCC(=O)NC4=O)C5=O)c3)c2)nnc1-c1ccncn1] | D52 |
| 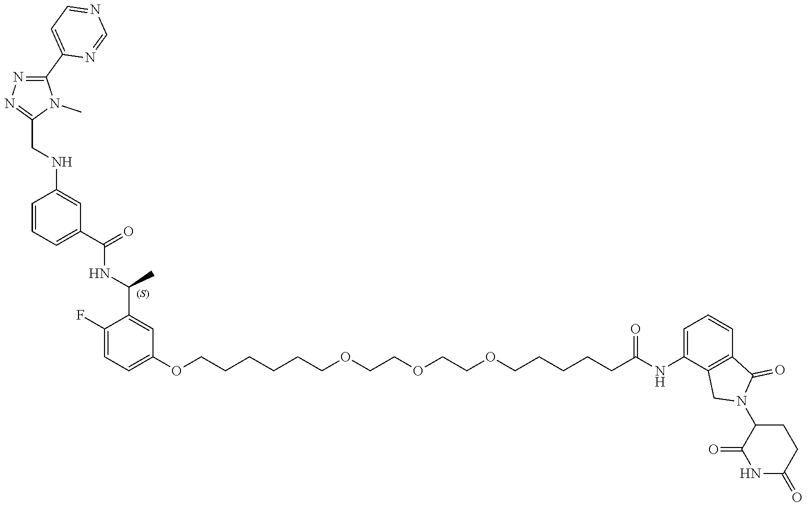 | D53 |
| 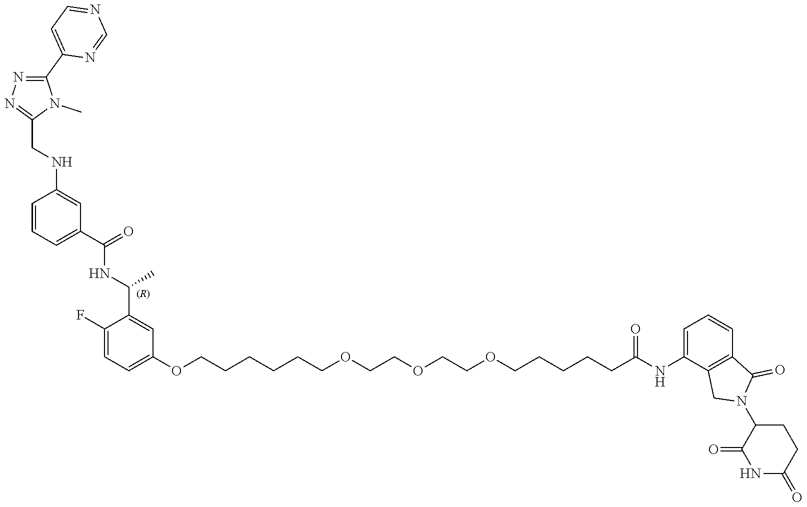 | D54 |
| 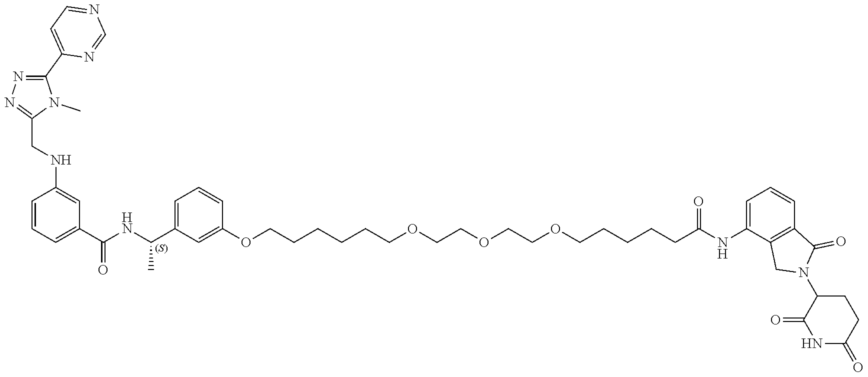 | D55 |
| 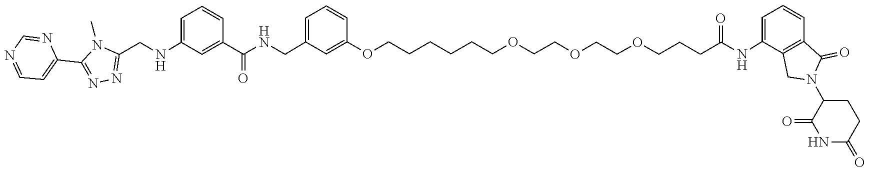 [SMILES: Cn1c(CNc2cccc(C(=O)NCc3cccc(OCCCCCCOCCOCCOCCCC(=O)Nc4cccc5c4CN(C4CCC(=O)NC4=O)C5=O)c3)c2)nnc1-c1ccncn1] | D56 |
| 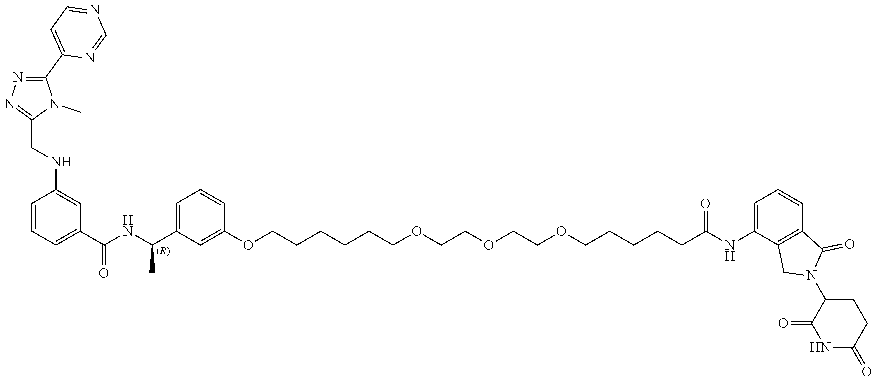 | D57 |
| 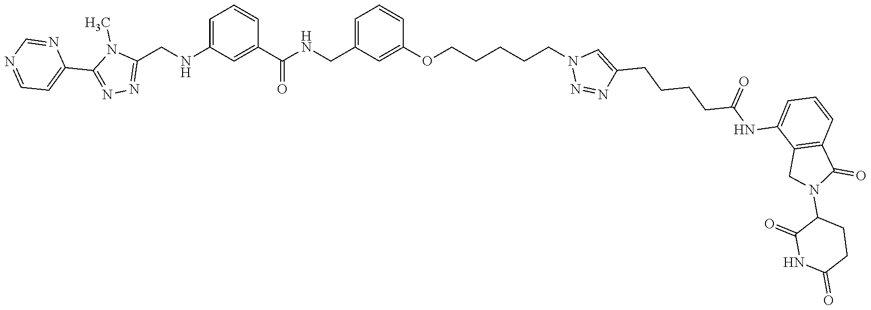 [SMILES: Cn1c(CNc2cccc(C(=O)NCc3cccc(OCCCCCn4cc(CCCCC(=O)Nc5cccc6c5CN(C5CCC(=O)NC5=O)C6=O)nn4)c3)c2)nnc1-c1ccncn1] | D58 |
| 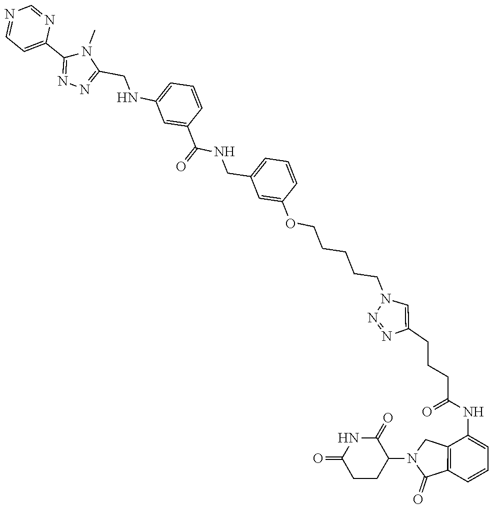 [SMILES: Cn1c(CNc2cccc(C(=O)NCc3cccc(OCCCCCn4cc(CCCC(=O)Nc5cccc6c5CN(C5CCC(=O)NC5=O)C6=O)nn4)c3)c2)nnc1-c1ccncn1] | D59 |
| 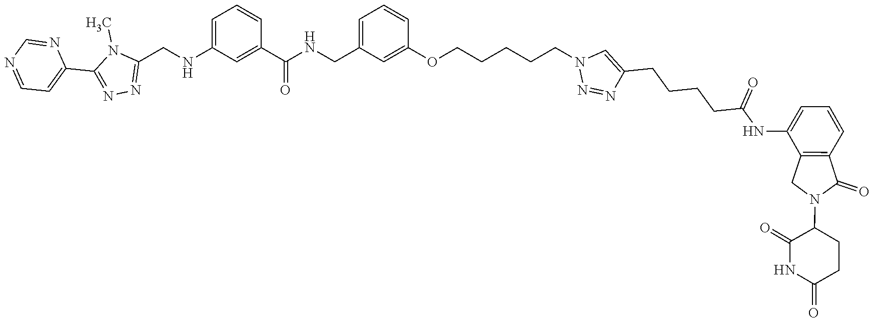 [SMILES: Cn1c(CNc2cccc(C(=O)NCc3cccc(OCCCCCn4cc(CCCCC(=O)Nc5cccc6c5CN(C5CCC(=O)NC5=O)C6=O)nn4)c3)c2)nnc1-c1ccncn1] | D60 |
| 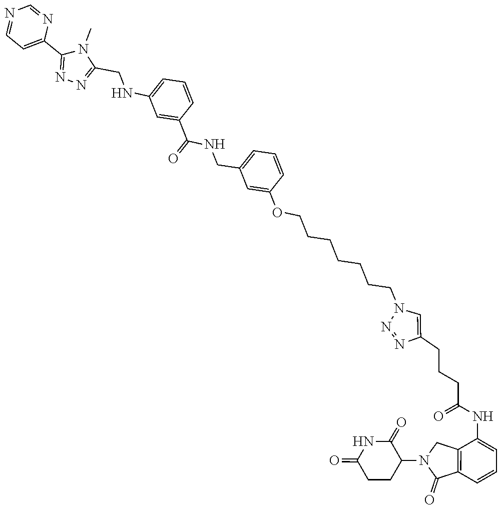 [SMILES: Cn1c(CNc2cccc(C(=O)NCc3cccc(OCCCCCCCn4cc(CCCC(=O)Nc5cccc6c5CN(C5CCC(=O)NC5=O)C6=O)nn4)c3)c2)nnc1-c1ccncn1] | D61 |
| 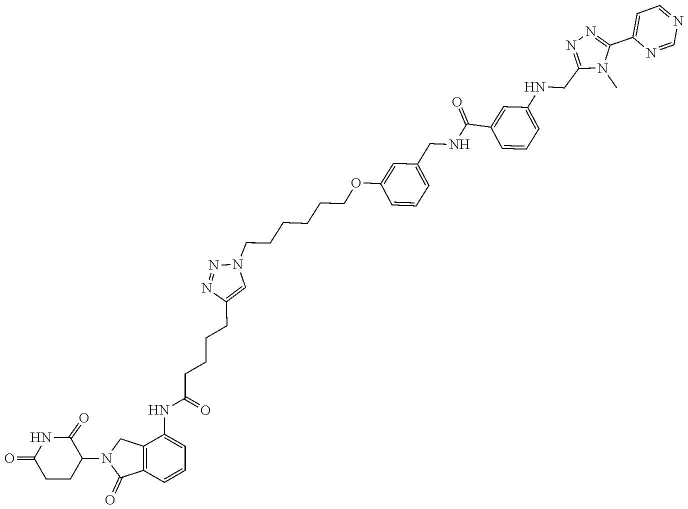 [SMILES: Cn1c(CNc2cccc(C(=O)NCc3cccc(OCCCCCCn4cc(CCCCC(=O)Nc5cccc6c5CN(C5CCC(=O)NC5=O)C6=O)nn4)c3)c2)nnc1-c1ccncn1] | D62 |
| 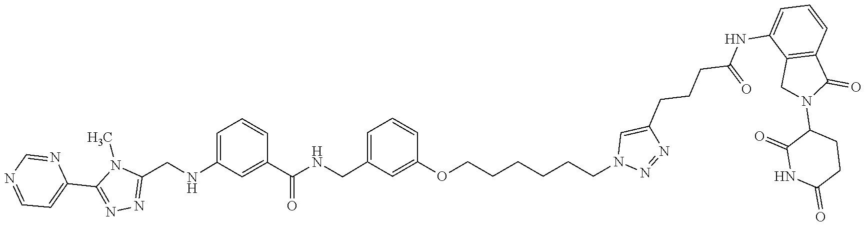 [SMILES: Cn1c(CNc2cccc(C(=O)NCc3cccc(OCCCCCCn4cc(CCCC(=O)Nc5cccc6c5CN(C5CCC(=O)NC5=O)C6=O)nn4)c3)c2)nnc1-c1ccncn1] | D63 |
| 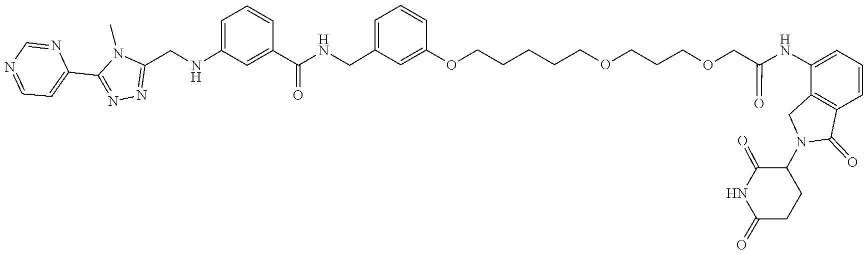 [SMILES: Cn1c(CNc2cccc(C(=O)NCc3cccc(OCCCCCOCCCOCC(=O)Nc4cccc5c4CN(C4CCC(=O)NC4=O)C5=O)c3)c2)nnc1-c1ccncn1] | D64 |
| 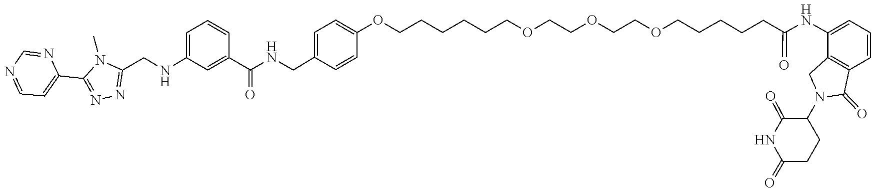 [SMILES: Cn1c(CNc2cccc(C(=O)NCc3ccc(OCCCCCCOCCOCCOCCCCCC(=O)Nc4cccc5c4CN(C4CCC(=O)NC4=O)C5=O)cc3)c2)nnc1-c1ccncn1] | D65 |
| 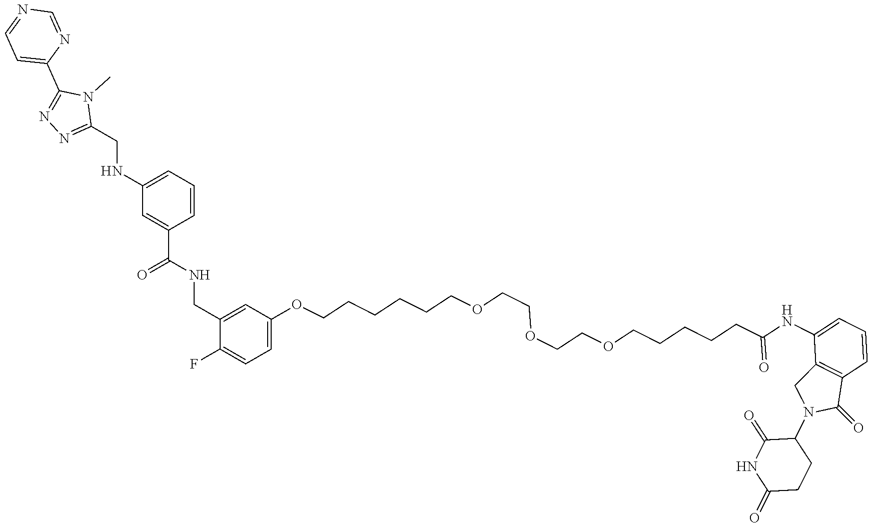 [SMILES: Cn1c(CNc2cccc(C(=O)NCc3cc(OCCCCCCOCCOCCOCCCCCC(=O)Nc4cccc5c4CN(C4CCC(=O)NC4=O)C5=O)ccc3F)c2)nnc1-c1ccncn1] | D66 |
| 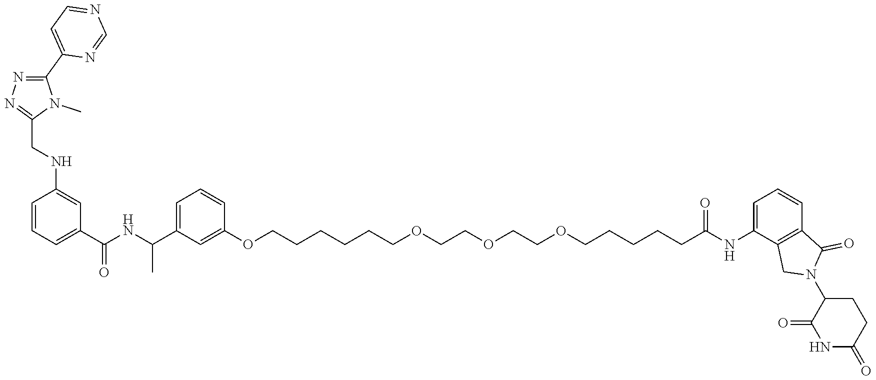 [SMILES: CC(NC(=O)c1cccc(NCc2nnc(-c3ccncn3)n2C)c1)c1cccc(OCCCCCCOCCOCCOCCCCCC(=O)Nc2cccc3c2CN(C2CCC(=O)NC2=O)C3=O)c1] | D67 |
| 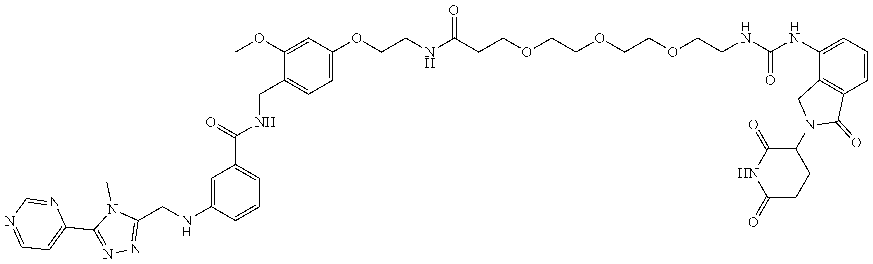 [SMILES: COc1cc(OCCNC(=O)CCOCCOCCOCCNC(=O)Nc2cccc3c2CN(C2CCC(=O)NC2=O)C3=O)ccc1CNC(=O)c1cccc(NCc2nnc(-c3ccncn3)n2C)c1] | D68 |
| 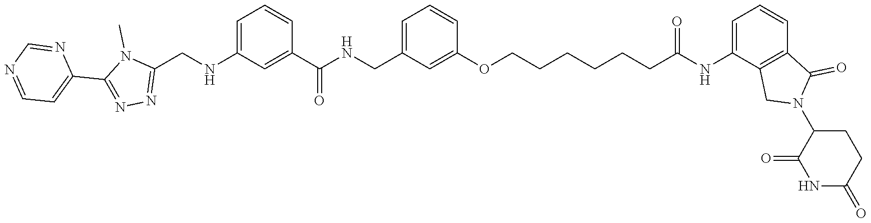 [SMILES: Cn1c(CNc2cccc(C(=O)NCc3cccc(OCCCCCCC(=O)Nc4cccc5c4CN(C4CCC(=O)NC4=O)C5=O)c3)c2)nnc1-c1ccncn1] | D69 |
| 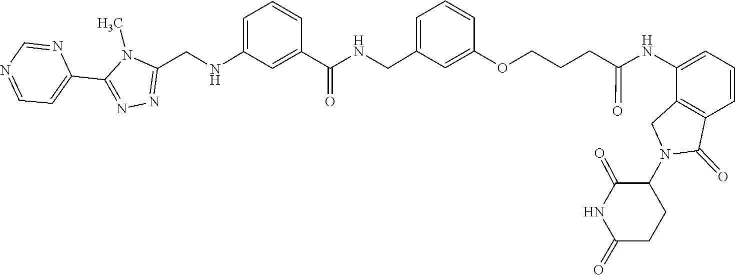 [SMILES: Cn1c(CNc2cccc(C(=O)NCc3cccc(OCCCC(=O)Nc4cccc5c4CN(C4CCC(=O)NC4=O)C5=O)c3)c2)nnc1-c1ccncn1] | D70 |
| 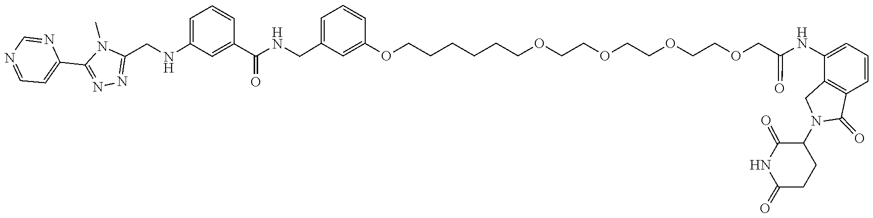 [SMILES: Cn1c(CNc2cccc(C(=O)NCc3cccc(OCCCCCCOCCOCCOCCOCC(=O)Nc4cccc5c4CN(C4CCC(=O)NC4=O)C5=O)c3)c2)nnc1-c1ccncn1] | D71 |
| 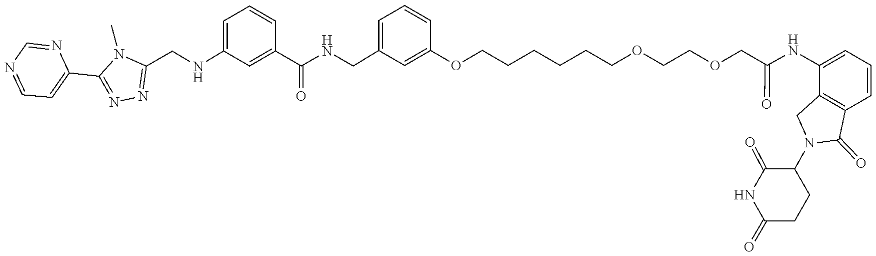 [SMILES: Cn1c(CNc2cccc(C(=O)NCc3cccc(OCCCCCCOCCOCC(=O)Nc4cccc5c4CN(C4CCC(=O)NC4=O)C5=O)c3)c2)nnc1-c1ccncn1] | D72 |
| 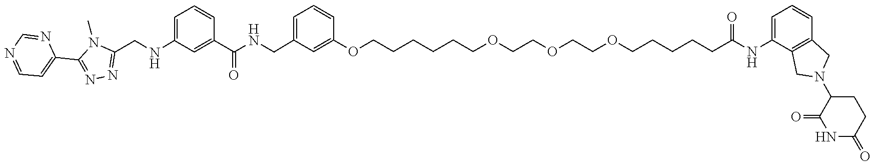 [SMILES: Cn1c(CNc2cccc(C(=O)NCc3cccc(OCCCCCCOCCOCCOCCCCCC(=O)Nc4cccc5c4CN(C4CCC(=O)NC4=O)C5)c3)c2)nnc1-c1ccncn1] | D73 |
| 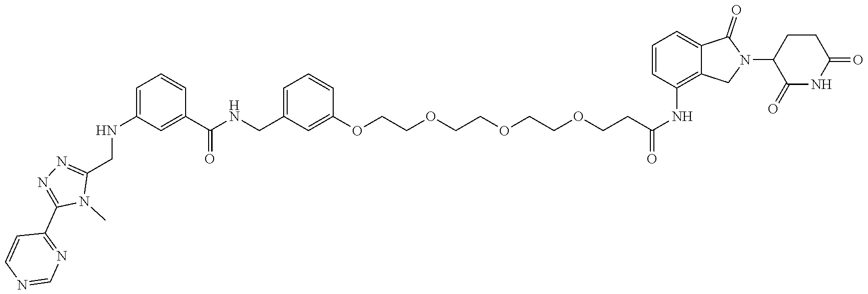 [SMILES: Cn1c(CNc2cccc(C(=O)NCc3cccc(OCCOCCOCCOCCC(=O)Nc4cccc5c4CN(C4CCC(=O)NC4=O)C5=O)c3)c2)nnc1-c1ccncn1] | D107 |
| 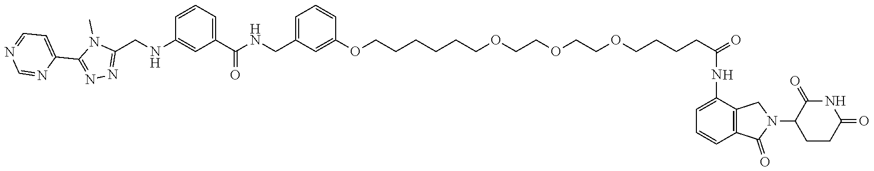 | D108 |
| 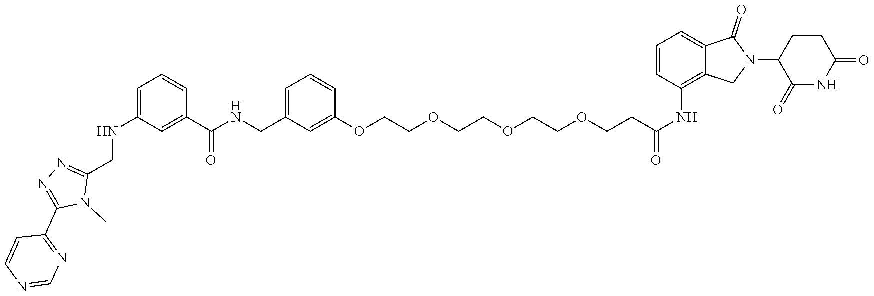 [SMILES: Cn1c(CNc2cccc(C(=O)NCc3cccc(OCCOCCOCCOCCC(=O)Nc4cccc5c4CN(C4CCC(=O)NC4=O)C5=O)c3)c2)nnc1-c1ccncn1] | D111 |
| 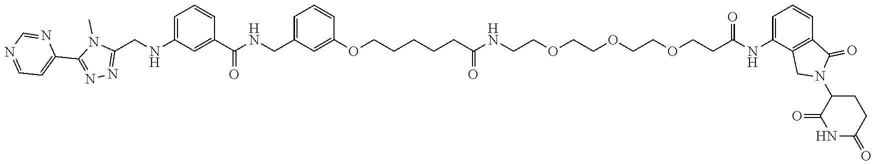 [SMILES: Cn1c(CNc2cccc(C(=O)NCc3cccc(OCCCCCC(=O)NCCOCCOCCOCCC(=O)Nc4cccc5c4CN(C4CCC(=O)NC4=O)C5=O)c3)c2)nnc1-c1ccncn1] | D113 |
| 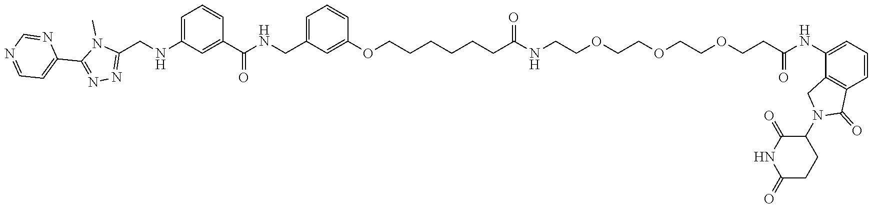 [SMILES: Cn1c(CNc2cccc(C(=O)NCc3cccc(OCCCCCCC(=O)NCCOCCOCCOCCC(=O)Nc4cccc5c4CN(C4CCC(=O)NC4=O)C5=O)c3)c2)nnc1-c1ccncn1] | D114 |
| 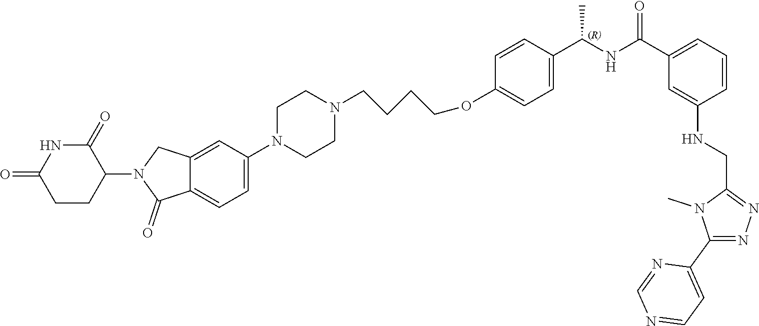 | D117 |
| 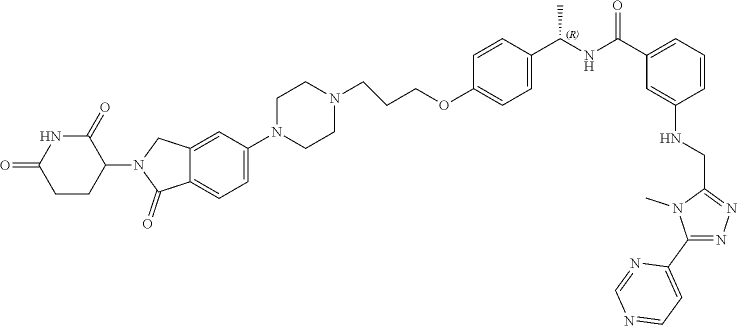 | D118 |
| 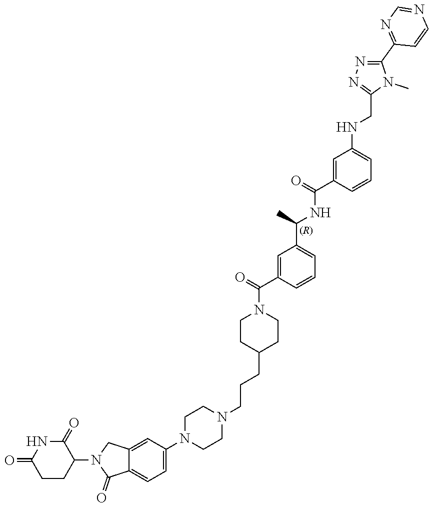 | D119 |
| 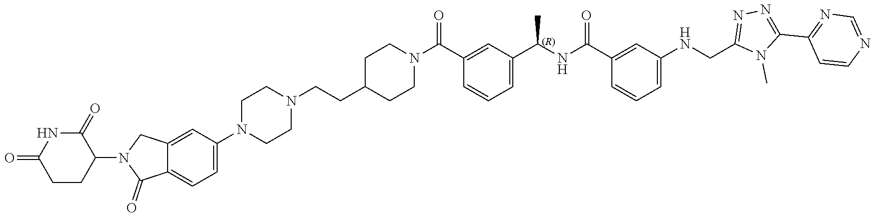 | D120 |
| 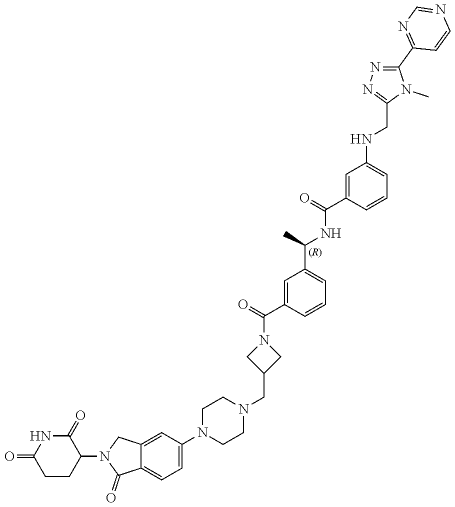 | D121 |
| 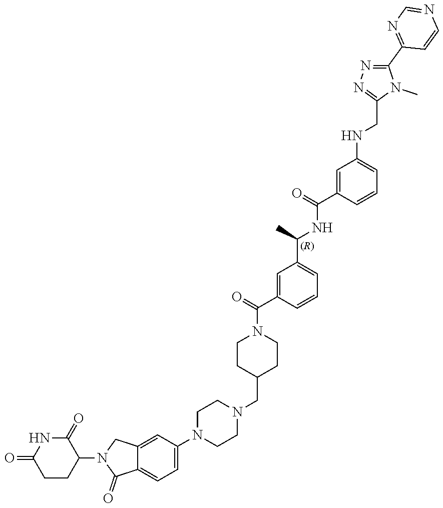 | D122 |
| 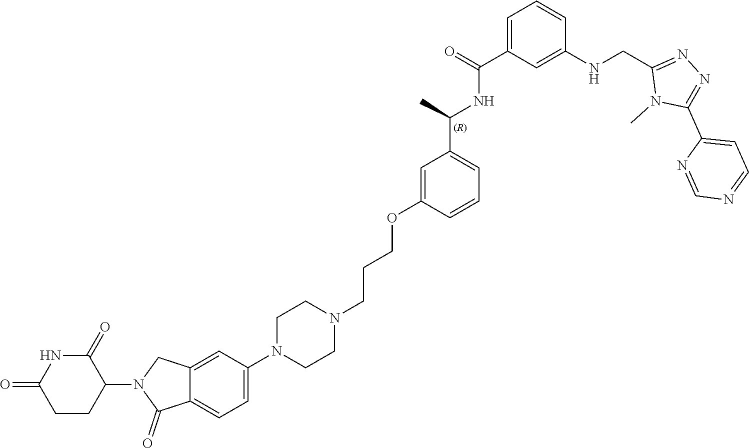 | D123 |
| 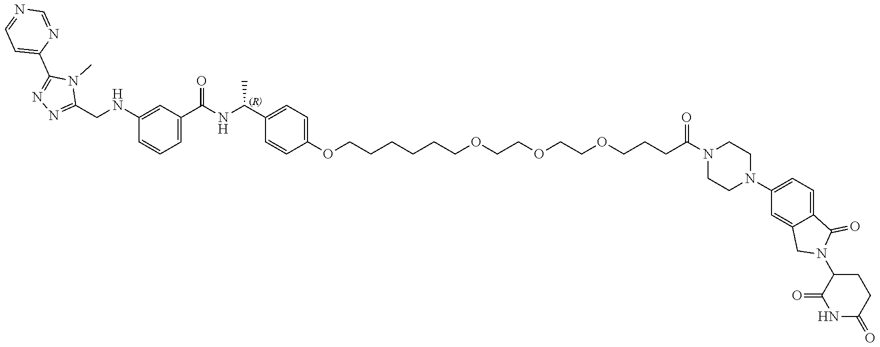 | D124 |
| 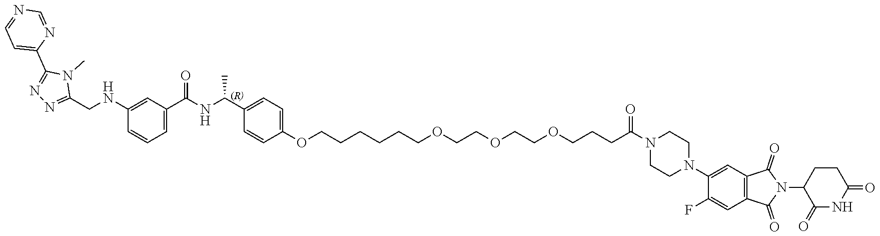 | D125 |
| 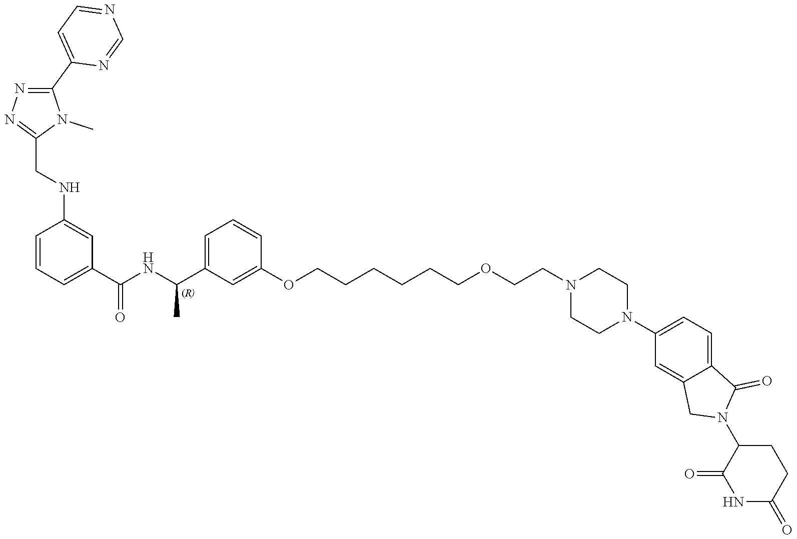 | D126 |
| 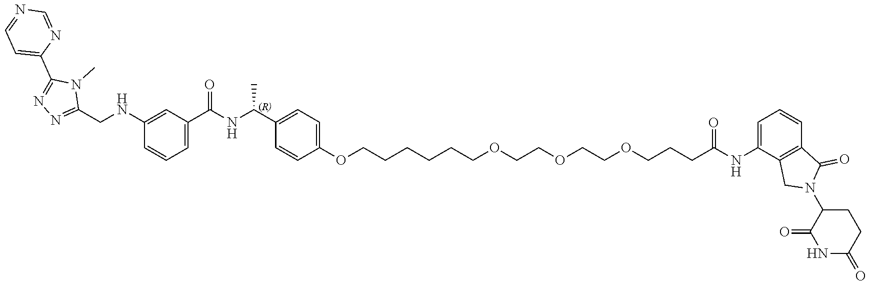 | D127 |
| 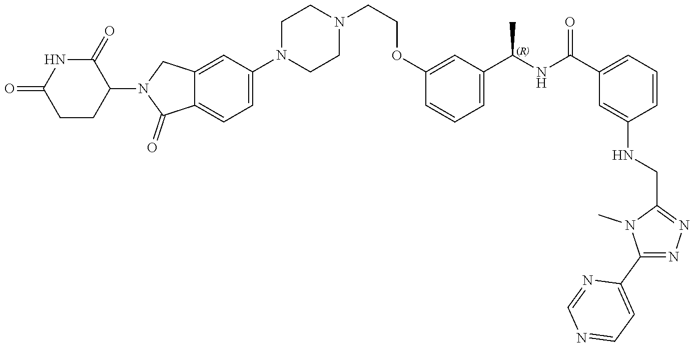 | D128 |
| 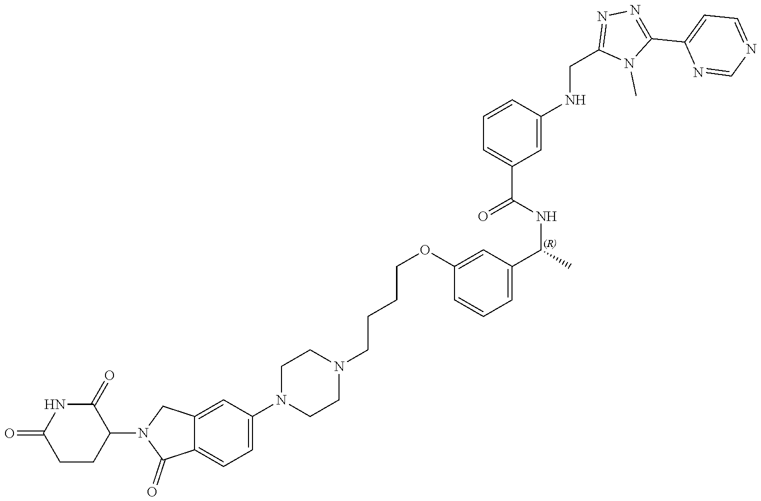 | D129 |
| 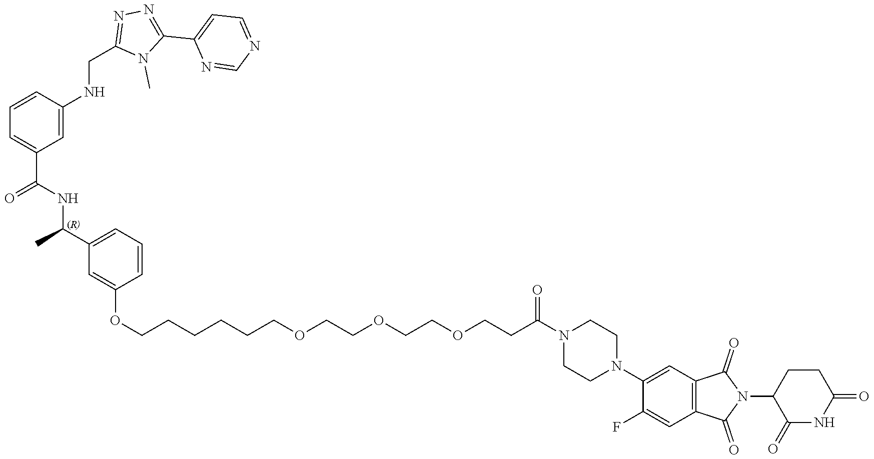 | D130 |
| 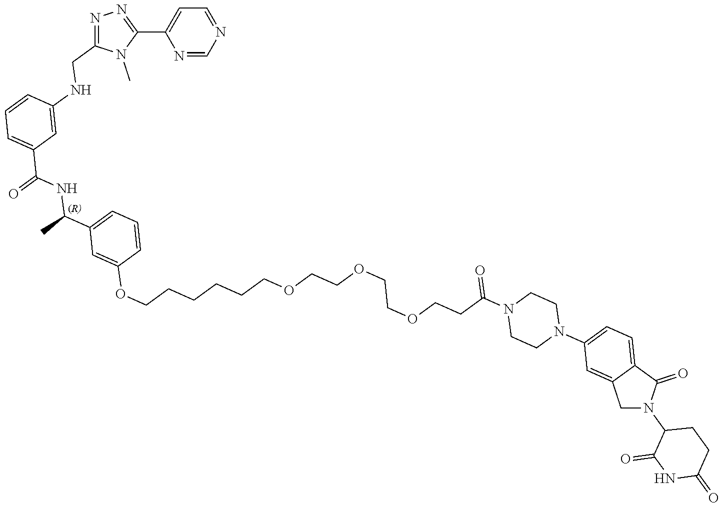 | D131 |
| 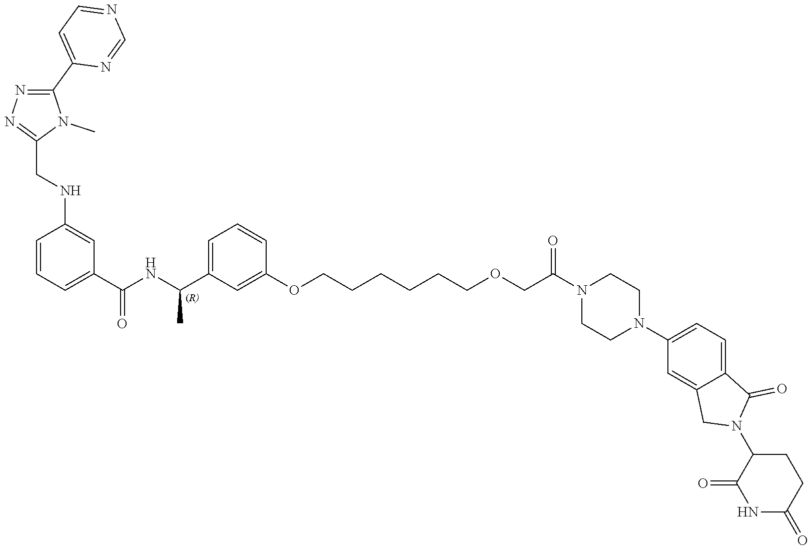 | D132 |
| 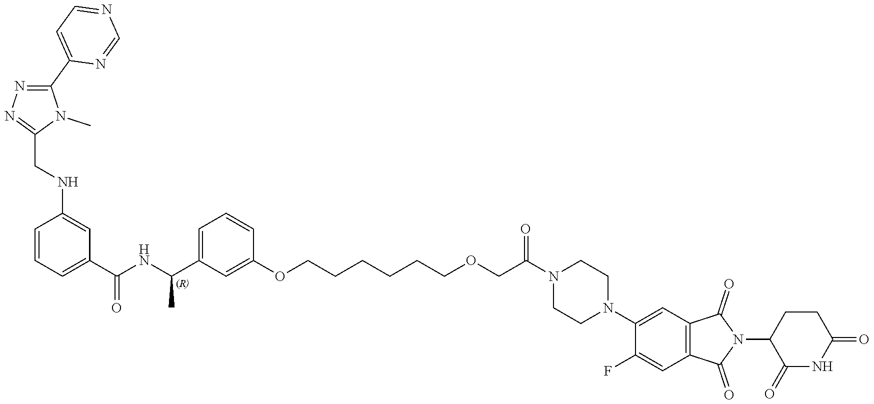 | D133 |
| 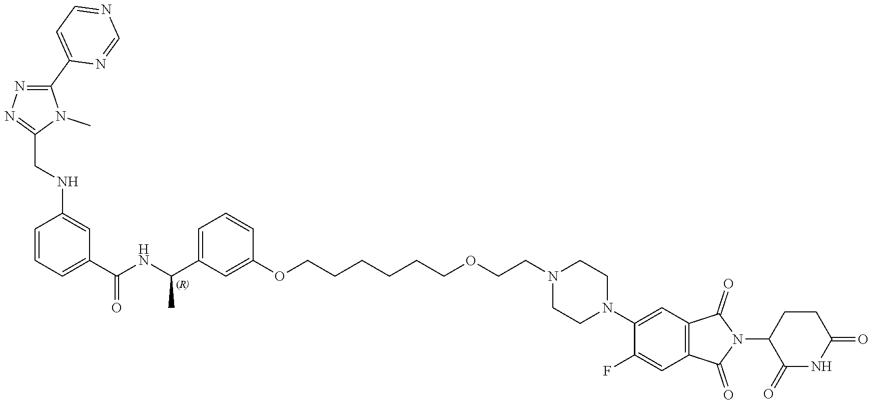 | D134 |
| 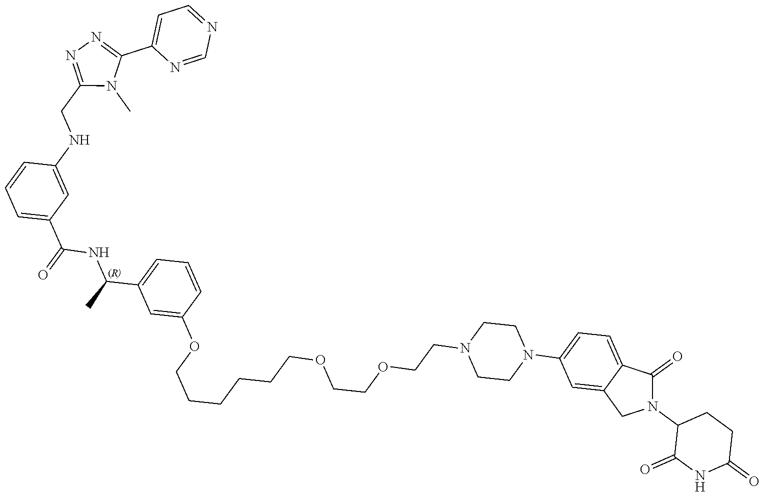 | D135 |
| 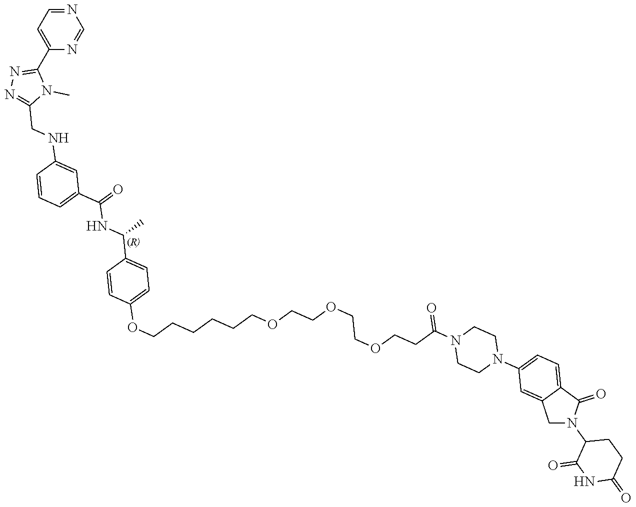 | D136 |
| 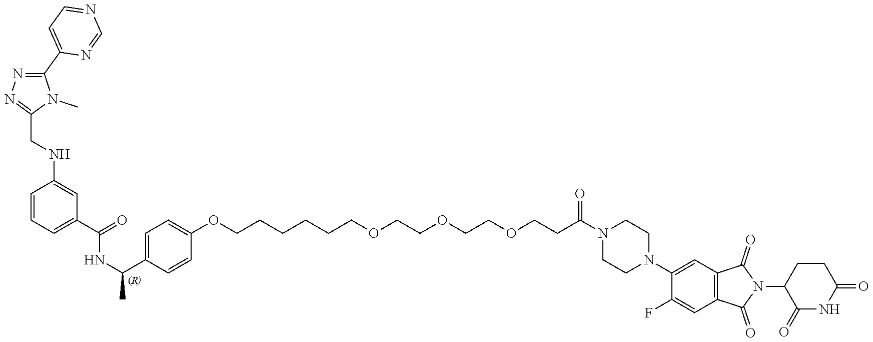 | D137 |
| 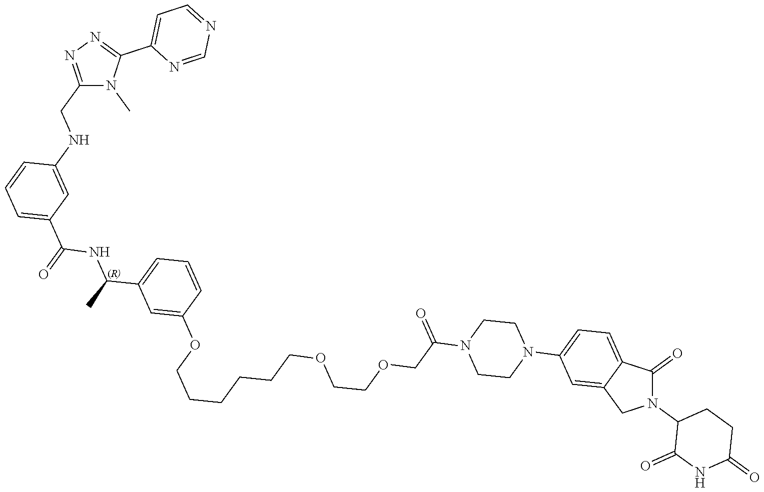 | D138 |
| 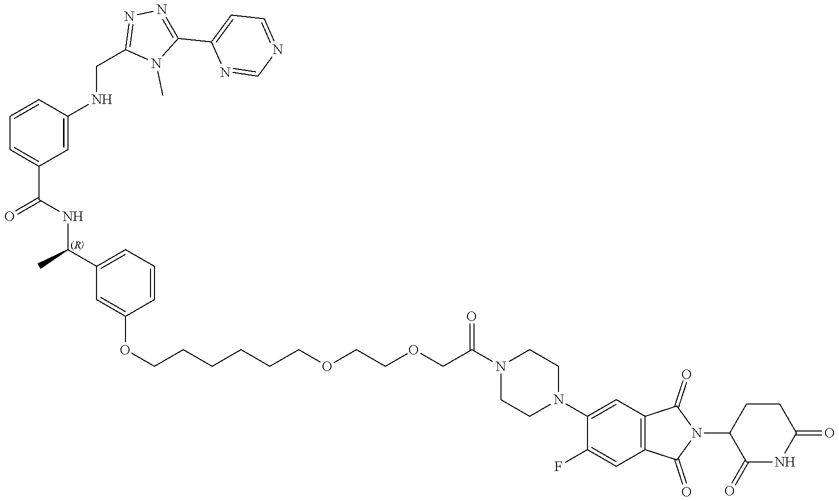 | D139 |
| 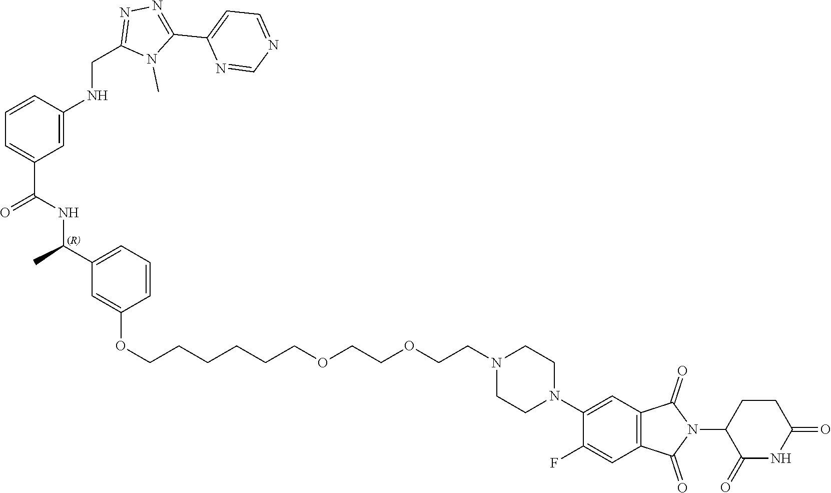 | D140 |
| 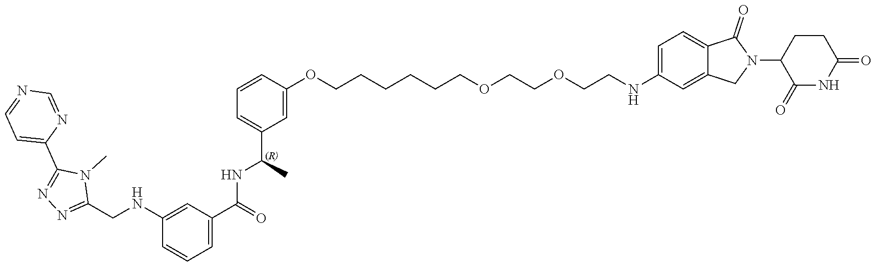 | D141 |
| 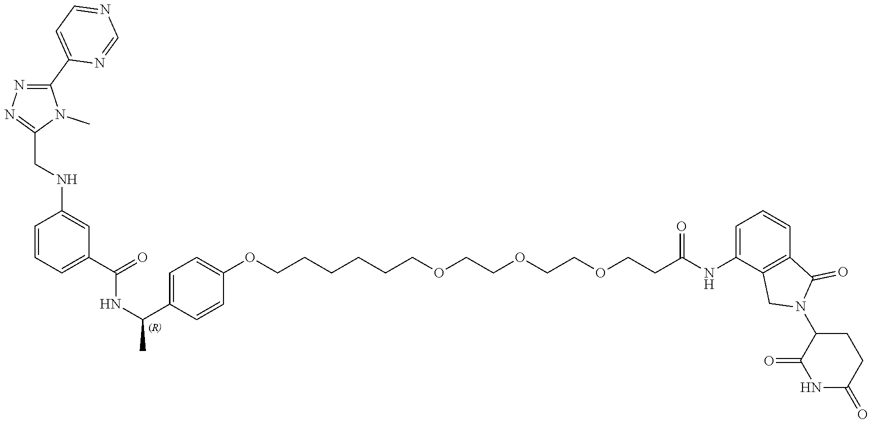 | D142 |
| 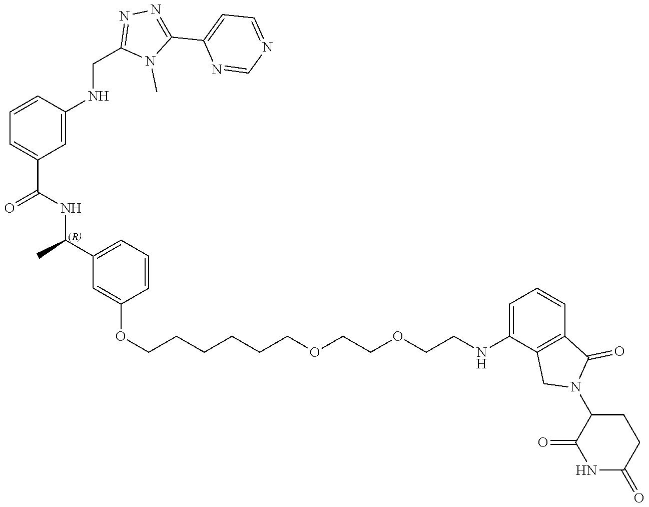 | D143 |
| 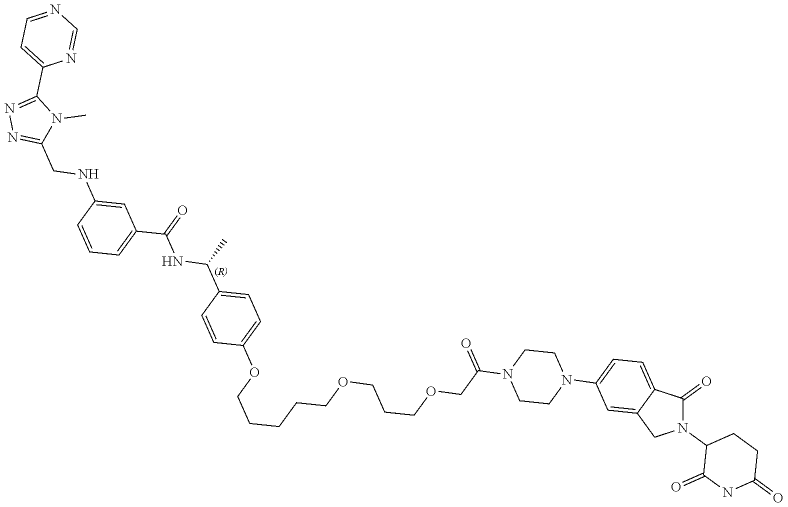 | D144 |
| 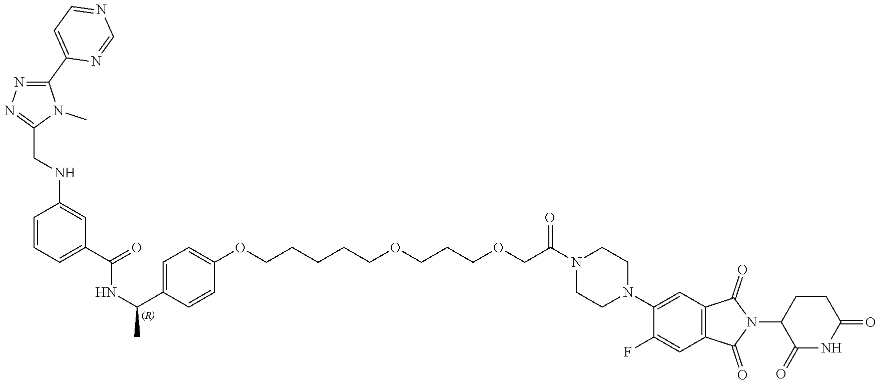 | D145 |
| 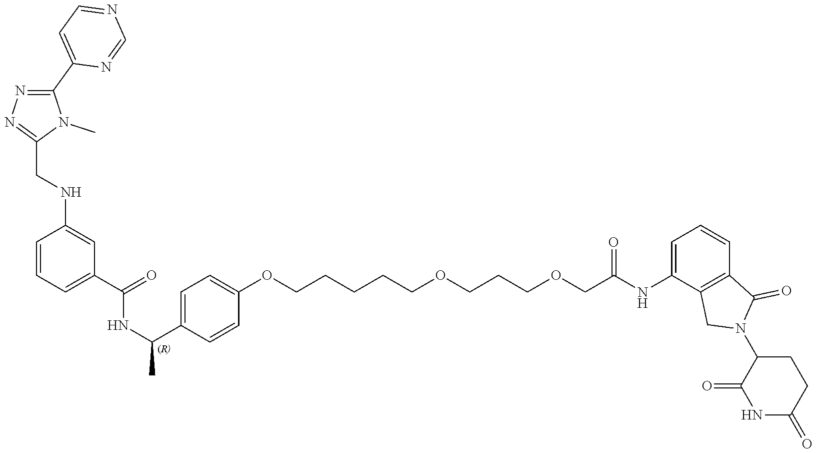 | D146 |
| 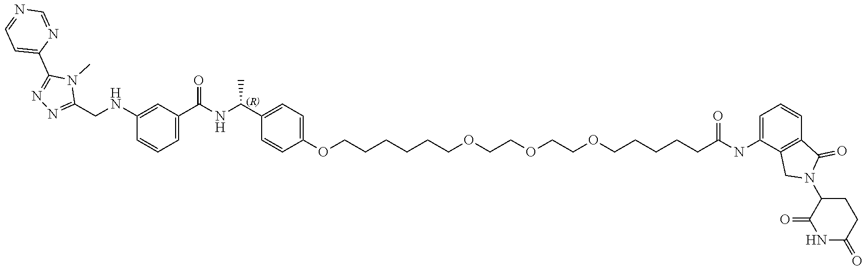 | D147 |
| 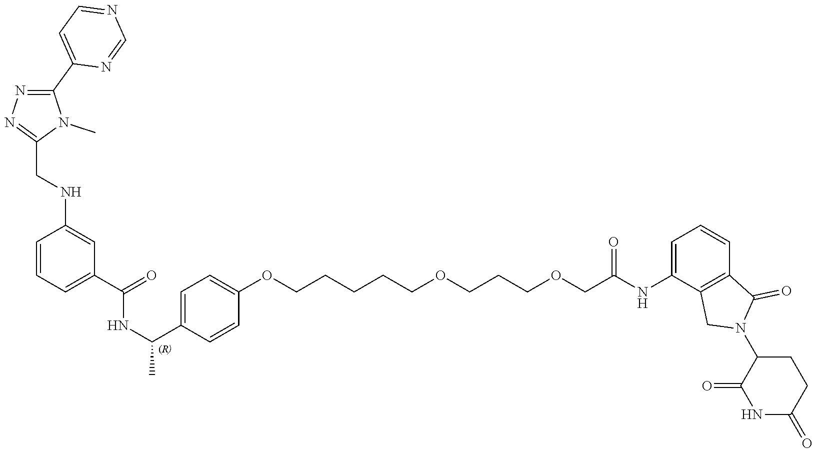 | D150 |
| 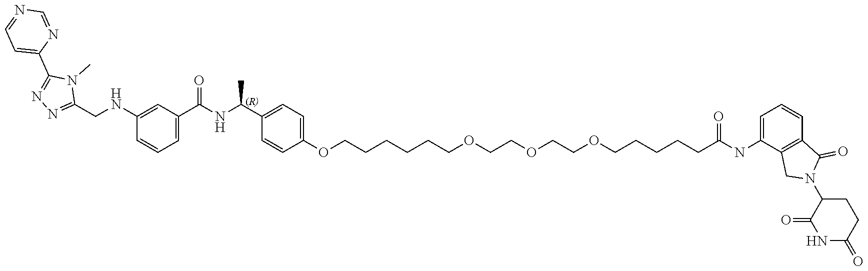 | D152 |
| 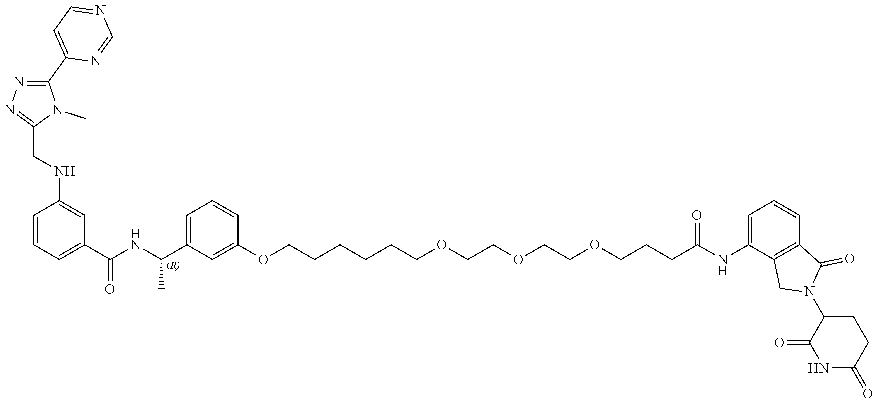 | D154 |
| 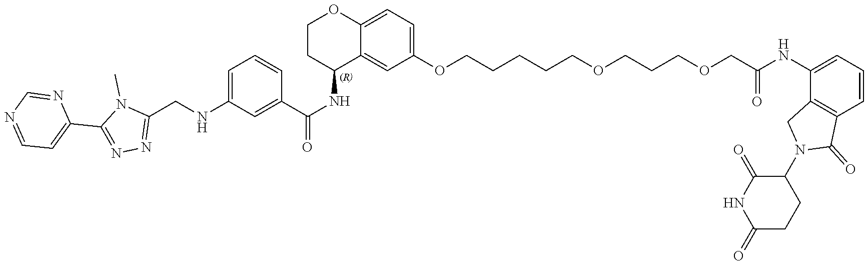 | D155 |
| 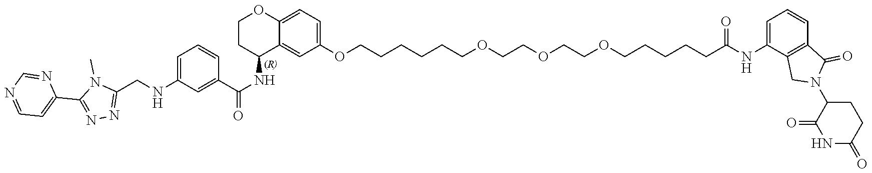 | D156 |
| 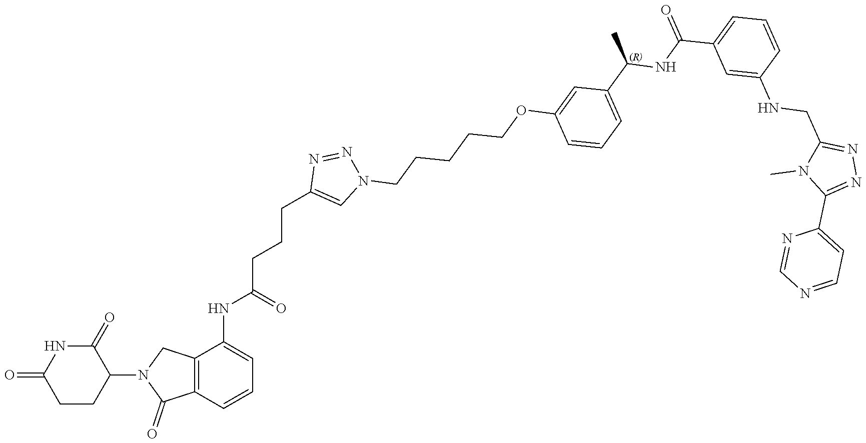 | D157 |
| 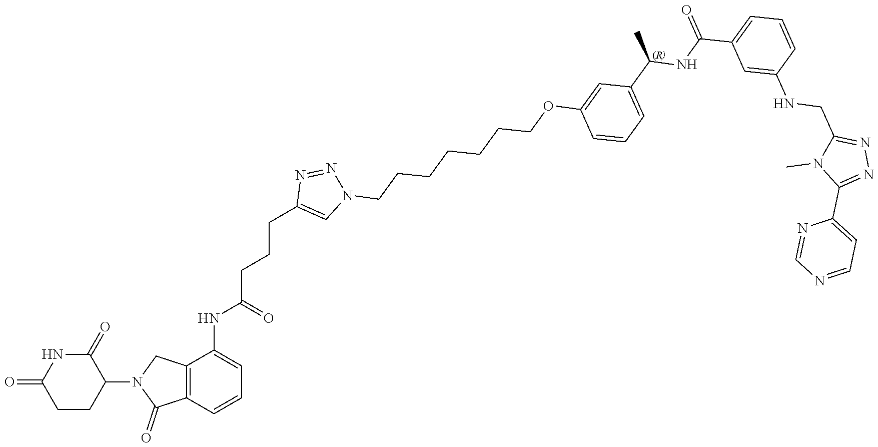 | D158 |
| 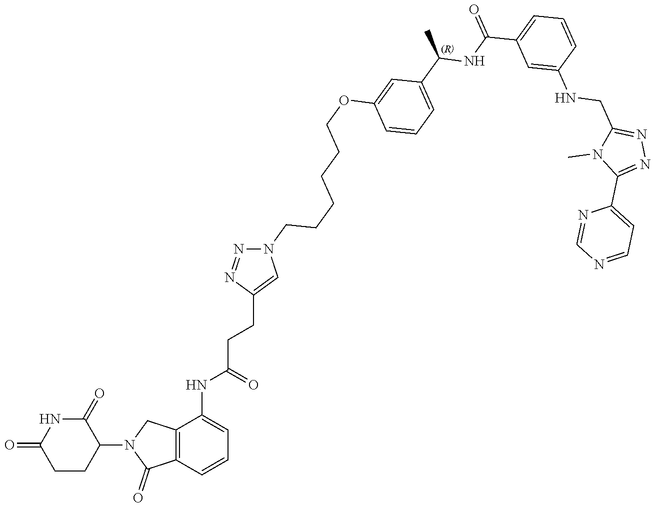 | D159 |
| 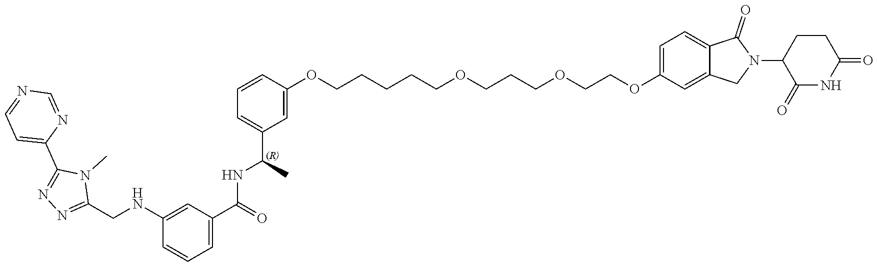 | D160 |
| 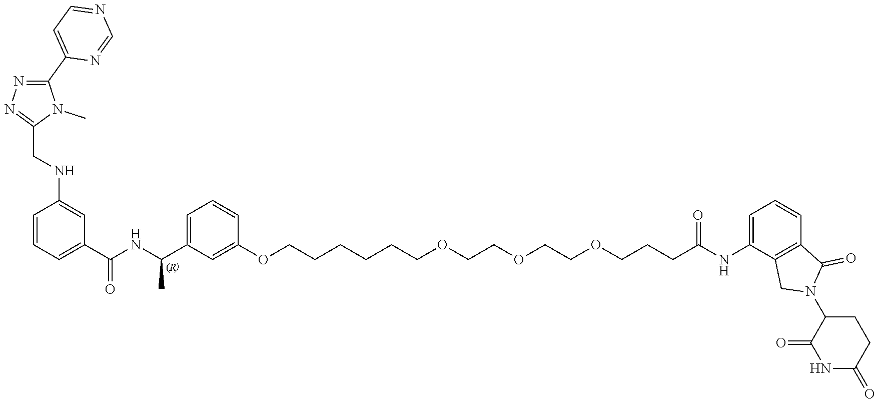 | D163 |
| 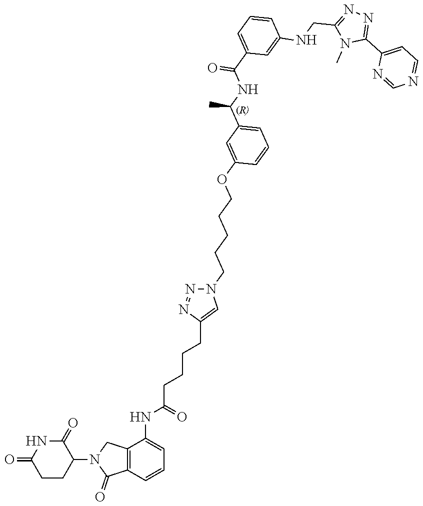 | D164 |
| 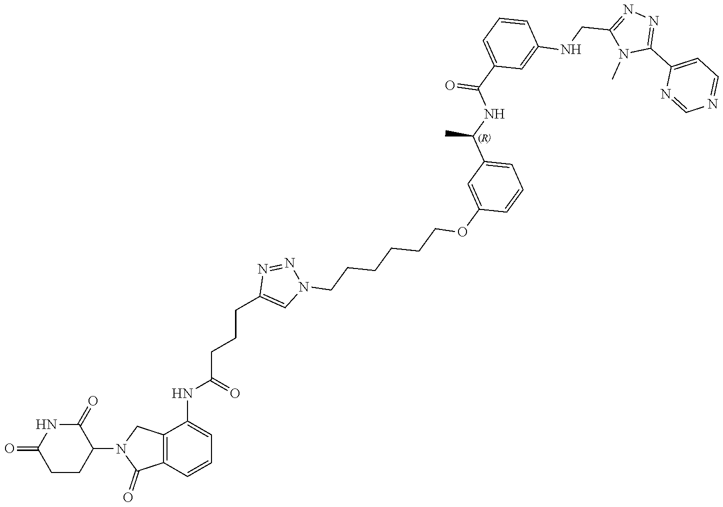 | D165 |
| 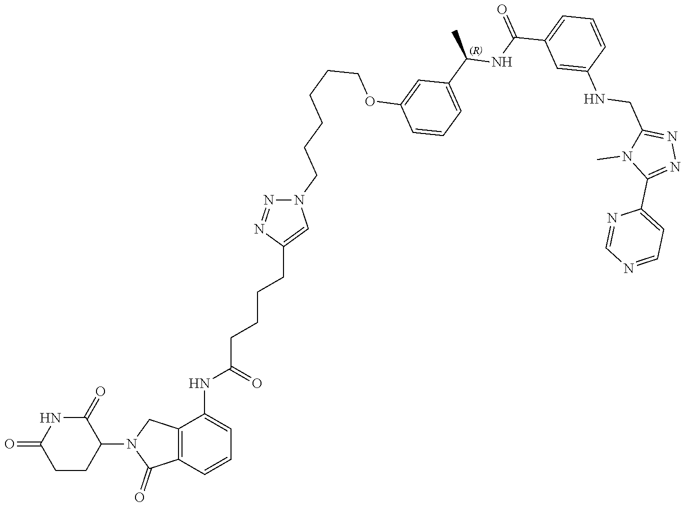 | D166 |
| 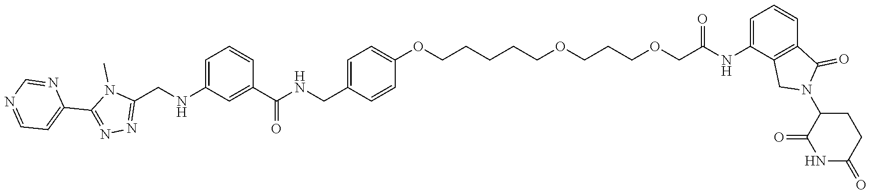 [SMILES: Cn1c(CNc2cccc(C(=O)NCc3ccc(OCCCCCOCCCOCC(=O)Nc4cccc5c4CN(C4CCC(=O)NC4=O)C5=O)cc3)c2)nnc1-c1ccncn1] | D167 |
| 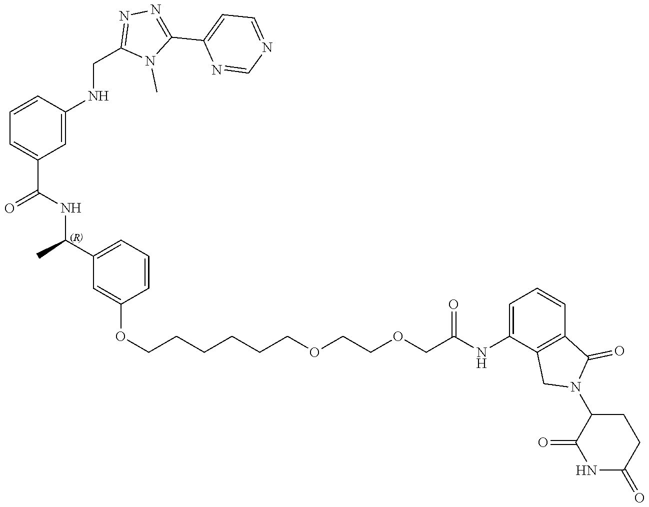 | D170 |
| 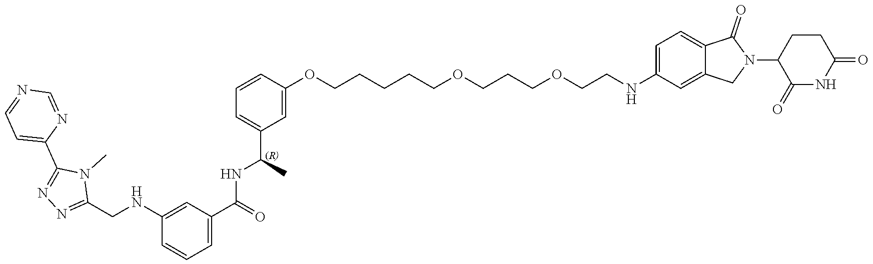 | D171 |
| 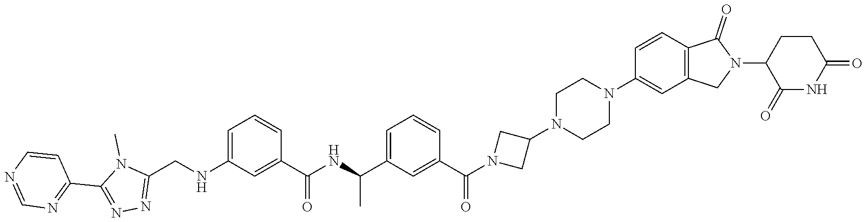 [SMILES: C[C@@H](NC(=O)c1cccc(NCc2nnc(-c3ccncn3)n2C)c1)c1cccc(C(=O)N2CC(N3CCN(c4ccc5c(c4)CN(C4CCC(=O)NC4=O)C5=O)CC3)C2)c1] | D172 |
| 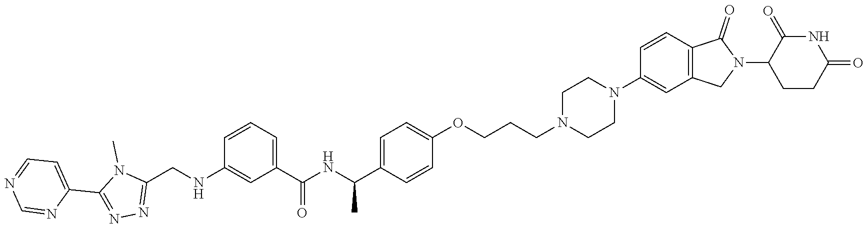 [SMILES: C[C@@H](NC(=O)c1cccc(NCc2nnc(-c3ccncn3)n2C)c1)c1ccc(OCCCN2CCN(c3ccc4c(c3)CN(C3CCC(=O)NC3=O)C4=O)CC2)cc1] | D173 |
| 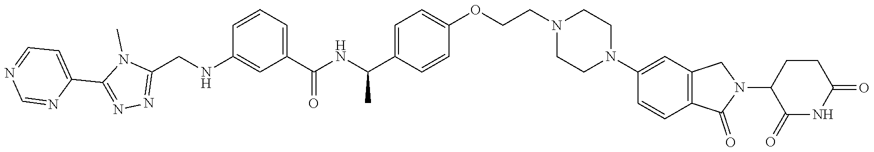 [SMILES: C[C@@H](NC(=O)c1cccc(NCc2nnc(-c3ccncn3)n2C)c1)c1ccc(OCCN2CCN(c3ccc4c(c3)CN(C3CCC(=O)NC3=O)C4=O)CC2)cc1] | D174 |
| 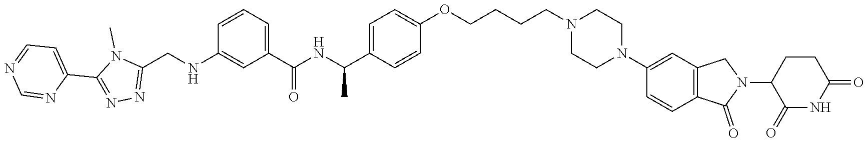 [SMILES: C[C@@H](NC(=O)c1cccc(NCc2nnc(-c3ccncn3)n2C)c1)c1ccc(OCCCCN2CCN(c3ccc4c(c3)CN(C3CCC(=O)NC3=O)C4=O)CC2)cc1] | D175 |
| 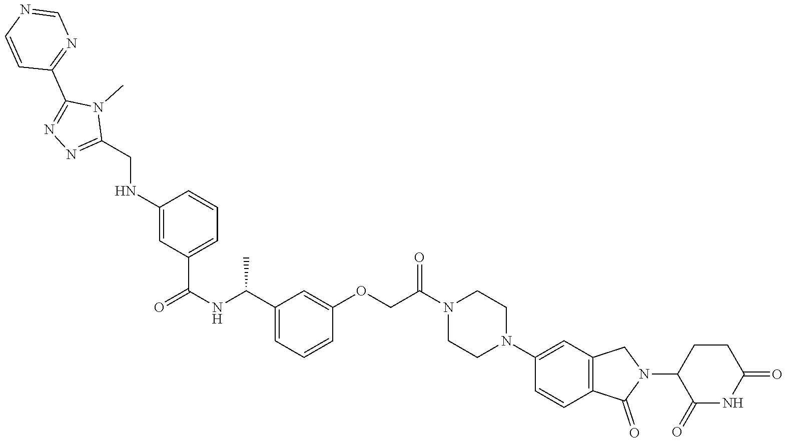 [SMILES: C[C@@H](NC(=O)c1cccc(NCc2nnc(-c3ccncn3)n2C)c1)c1cccc(OCC(=O)N2CCN(c3ccc4c(c3)CN(C3CCC(=O)NC3=O)C4=O)CC2)c1] | D176 |
| 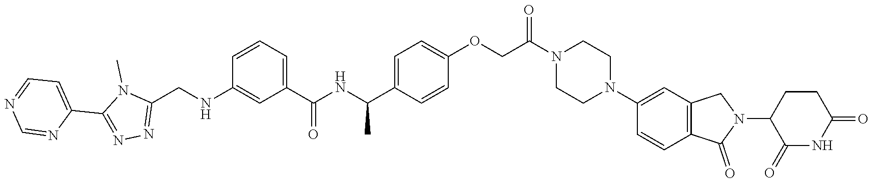 [SMILES: C[C@@H](NC(=O)c1cccc(NCc2nnc(-c3ccncn3)n2C)c1)c1ccc(OCC(=O)N2CCN(c3ccc4c(c3)CN(C3CCC(=O)NC3=O)C4=O)CC2)cc1] | D177 |
| 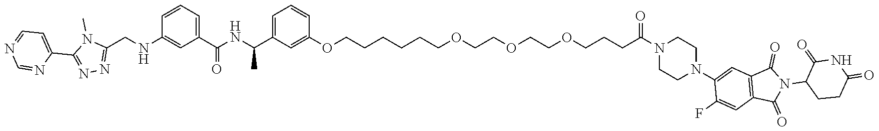 [SMILES: C[C@@H](NC(=O)c1cccc(NCc2nnc(-c3ccncn3)n2C)c1)c1cccc(OCCCCCCOCCOCCOCCCC(=O)N2CCN(c3cc4c(cc3F)C(=O)N(C3CCC(=O)NC3=O)C4=O)CC2)c1] | D178 |
| 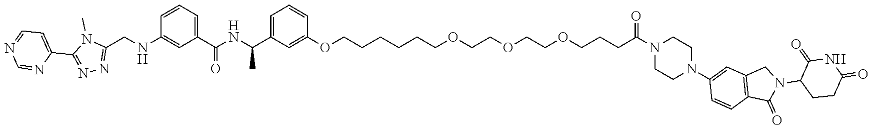 [SMILES: C[C@@H](NC(=O)c1cccc(NCc2nnc(-c3ccncn3)n2C)c1)c1cccc(OCCCCCCOCCOCCOCCCC(=O)N2CCN(c3ccc4c(c3)CN(C3CCC(=O)NC3=O)C4=O)CC2)c1] | D179 |
| 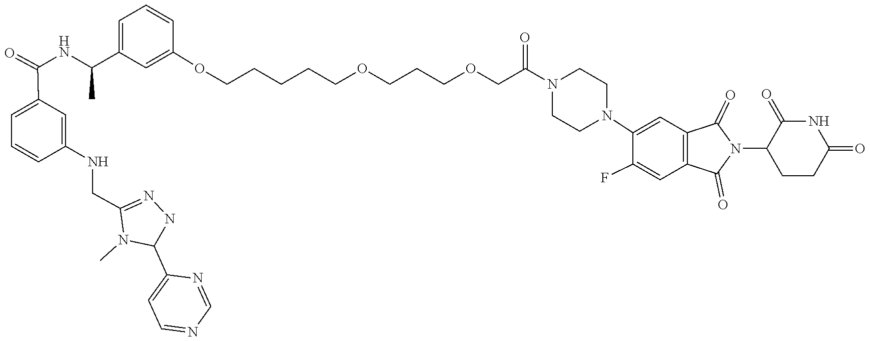 | D180 |
| 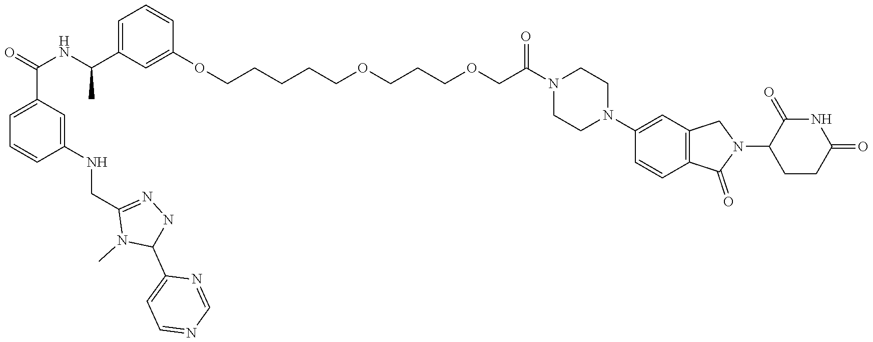 | D181 |
| 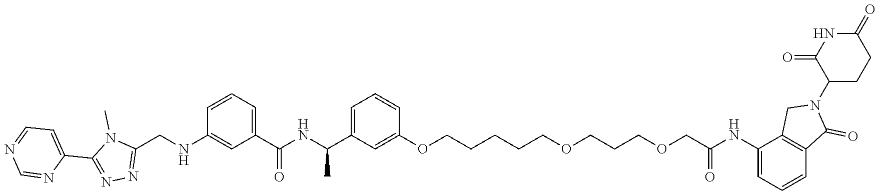 [SMILES: C[C@@H](NC(=O)c1cccc(NCc2nnc(-c3ccncn3)n2C)c1)c1cccc(OCCCCCOCCCOCC(=O)Nc2cccc3c2CN(C2CCC(=O)NC2=O)C3=O)c1] | D183 |
| 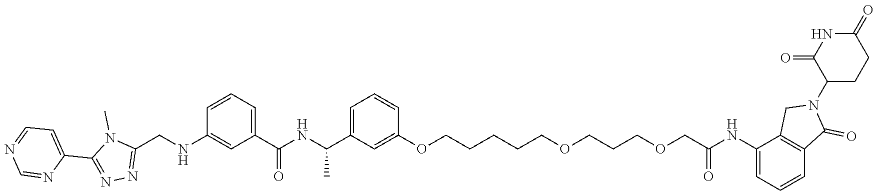 [SMILES: C[C@H](NC(=O)c1cccc(NCc2nnc(-c3ccncn3)n2C)c1)c1cccc(OCCCCCOCCCOCC(=O)Nc2cccc3c2CN(C2CCC(=O)NC2=O)C3=O)c1] | D185 |
| 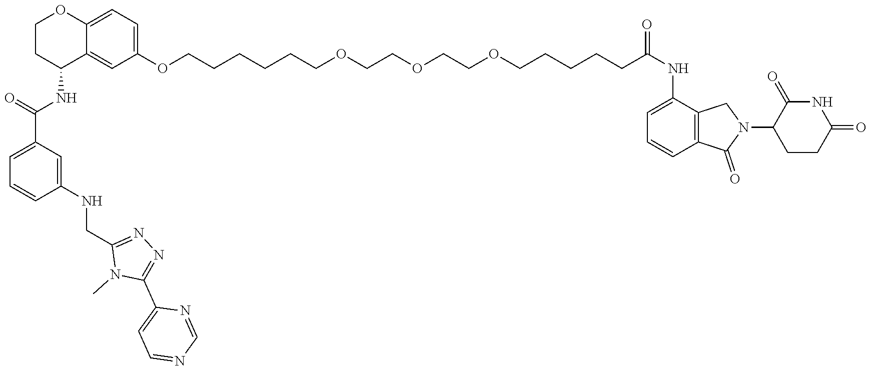 [SMILES: Cn1c(CNc2cccc(C(=O)N[C@@H]3CCOc4ccc(OCCCCCCOCCOCCOCCCCCC(=O)Nc5cccc6c5CN(C5CCC(=O)NC5=O)C6=O)cc43)c2)nnc1-c1ccncn1] | D187 |
| 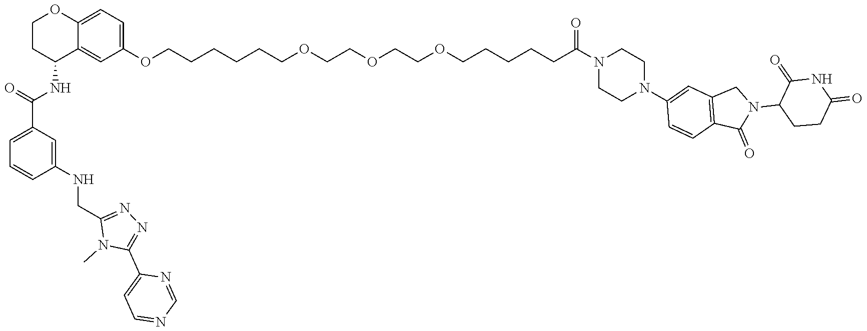 [SMILES: Cn1c(CNc2cccc(C(=O)N[C@@H]3CCOc4ccc(OCCCCCCOCCOCCOCCCCCC(=O)N5CCN(c6ccc7c(c6)CN(C6CCC(=O)NC6=O)C7=O)CC5)cc43)c2)nnc1-c1ccncn1] | D188 |
| 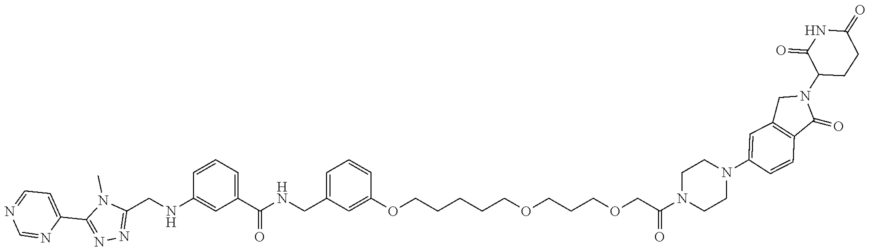 [SMILES: Cn1c(CNc2cccc(C(=O)NCc3cccc(OCCCCCOCCCOCC(=O)N4CCN(c5ccc6c(c5)CN(C5CCC(=O)NC5=O)C6=O)CC4)c3)c2)nnc1-c1ccncn1] | D189 |
| 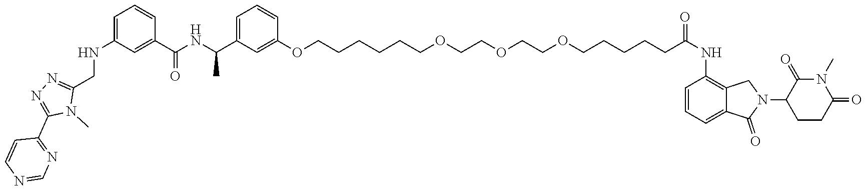 [SMILES: C[C@@H](NC(=O)c1cccc(NCc2nnc(-c3ccncn3)n2C)c1)c1cccc(OCCCCCCOCCOCCOCCCCCC(=O)Nc2cccc3c2CN(C2CCC(=O)N(C)C2=O)C3=O)c1] | D190 |
| 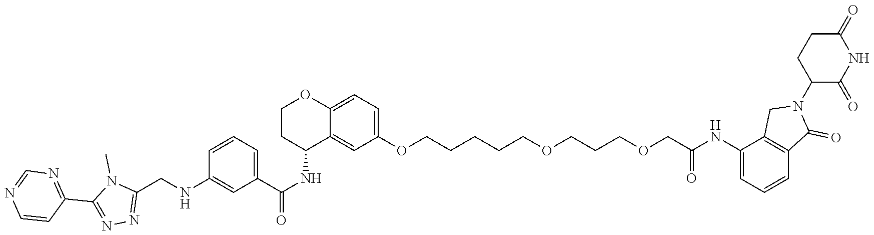 [SMILES: Cn1c(CNc2cccc(C(=O)N[C@@H]3CCOc4ccc(OCCCCCOCCCOCC(=O)Nc5cccc6c5CN(C5CCC(=O)NC5=O)C6=O)cc43)c2)nnc1-c1ccncn1] | D192 |
| 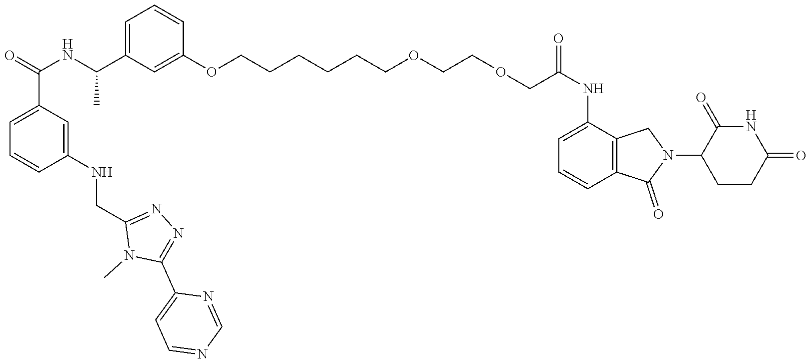 [SMILES: C[C@H](NC(=O)c1cccc(NCc2nnc(-c3ccncn3)n2C)c1)c1cccc(OCCCCCCOCCOCC(=O)Nc2cccc3c2CN(C2CCC(=O)NC2=O)C3=O)c1] | D193 |
| 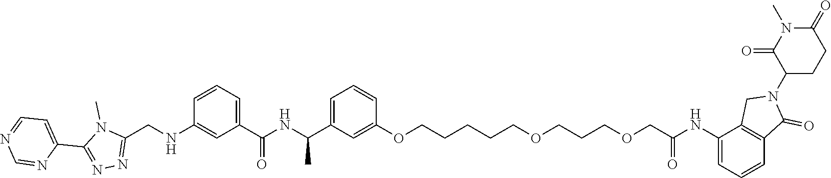 [SMILES: C[C@@H](NC(=O)c1cccc(NCc2nnc(-c3ccncn3)n2C)c1)c1cccc(OCCCCCOCCCOCC(=O)Nc2cccc3c2CN(C2CCC(=O)N(C)C2=O)C3=O)c1] | D194 |
| 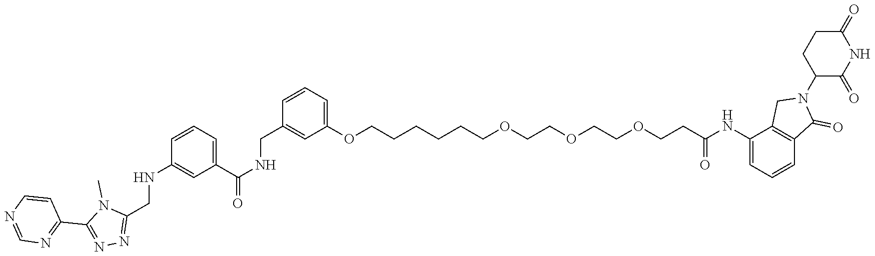 [SMILES: Cn1c(CNc2cccc(C(=O)NCc3cccc(OCCCCCCOCCOCCOCCC(=O)Nc4cccc5c4CN(C4CCC(=O)NC4=O)C5=O)c3)c2)nnc1-c1ccncn1] | D195 |
| 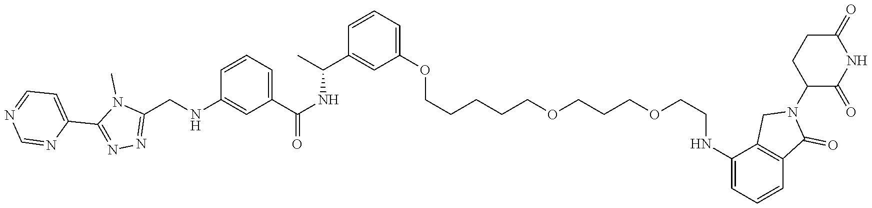 [SMILES: C[C@@H](NC(=O)c1cccc(NCc2nnc(-c3ccncn3)n2C)c1)c1cccc(OCCCCCOCCCOCCNc2cccc3c2CN(C2CCC(=O)NC2=O)C3=O)c1] | D196 |
| 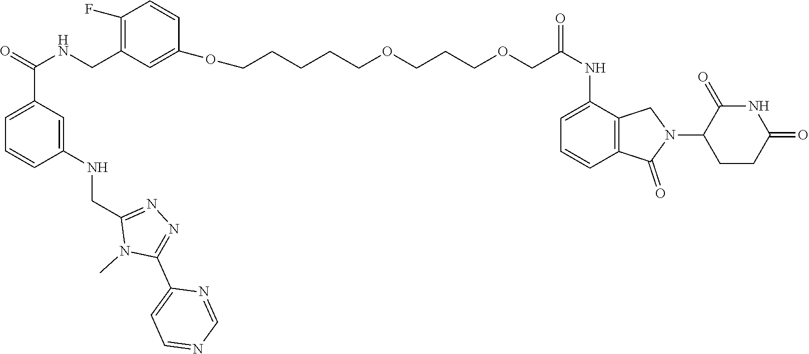 | D197 |
| 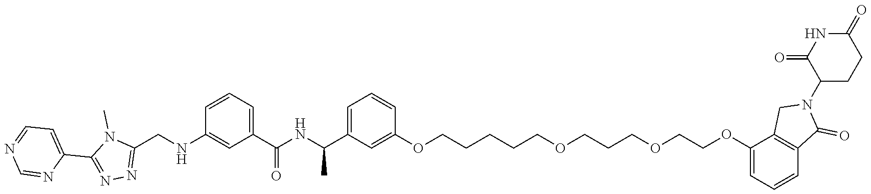 [SMILES: C[C@@H](NC(=O)c1cccc(NCc2nnc(-c3ccncn3)n2C)c1)c1cccc(OCCCCCOCCCOCCOc2cccc3c2CN(C2CCC(=O)NC2=O)C3=O)c1] | D198 |

Also provided herein are compounds of Formula (B-I):

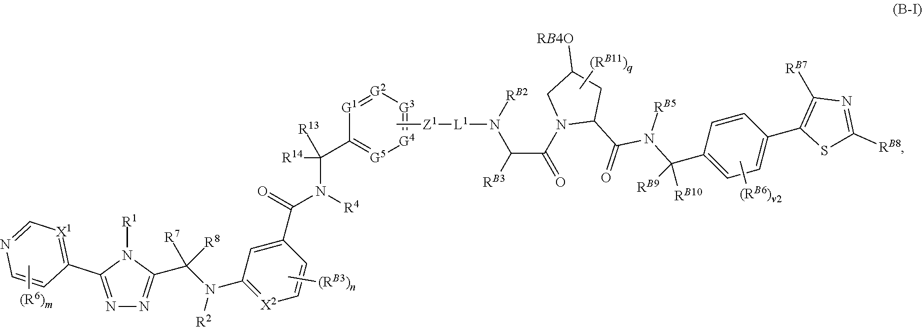

and pharmaceutically acceptable salts, stereoisomers, tautomers, isotopically labeled derivatives, solvates, hydrates, polymorphs, co-crystals, and prodrugs thereof, wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof;
Z¹ is a bond, —O—, —NR^{N}—, —S—, —C(═O)—, or optionally substituted —CH₂—;
G¹ is CR¹⁵ or N;
G², G³, G⁴, and G⁵ are each independently CR¹⁶, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{B2}, and R^{B5} are each independently hydrogen, optionally substituted C₁₋₆ alkyl, optionally substituted C₁₋₆ acyl, or a nitrogen protecting group;
each instance of R³, R⁶, R⁹, R¹⁵, R¹⁶, R^{B6}, R^{B7}, and R^{B8} is independently hydrogen, halogen, —CN, —N₃, —NO₂, optionally substituted C₁₋₆ alkyl, optionally substituted C₁₋₆ heteroalkyl, optionally substituted C₂₋₆alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R⁸ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{B3} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, or optionally substituted 5-10 membered heteroaryl;
R^{B4} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
R^{B9} and R^{B10} are independently hydrogen or optionally substituted C₁₋₆ alkyl, or optionally R^{B9} and R^{B10} are joined together with the intervening atoms to form optionally substituted C₃₋₈ carbocyclyl or optionally substituted 3-8 membered heterocyclyl;
each instance of R^{B11} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
m and n are each independently 0, 1, 2, or 3;
q is 0, 1, 2, 3, 4, or 5; and
v2 is 0, 1, 2, 3, or 4.

In certain embodiments, a compound of Formula (B-I) is of Formula (B-II):

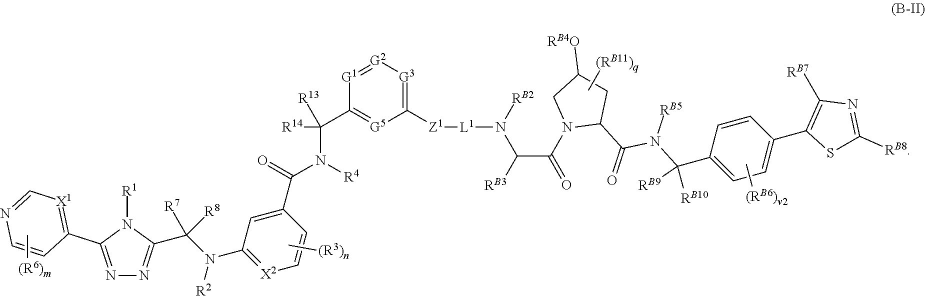

or a pharmaceutically acceptable salt, stereoisomer, tautomer, isotopically labeled derivative, solvate, hydrate, polymorph, co-crystal, or prodrug thereof.

In certain embodiments, a compound of Formula (B-I) is of Formula (B-III):

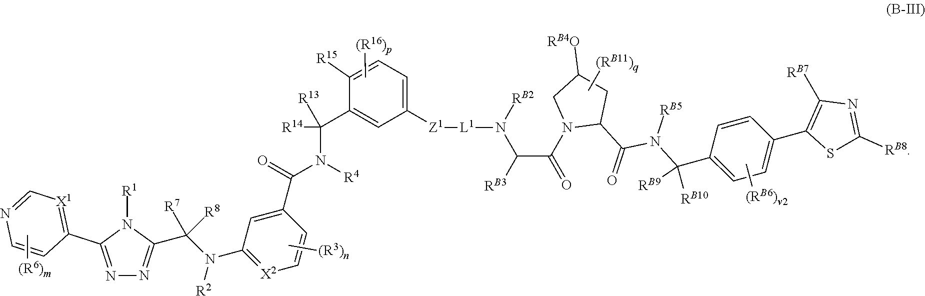

or a pharmaceutically acceptable salt, stereoisomer, tautomer, isotopically labeled derivative, solvate, hydrate, polymorph, co-crystal, or prodrug thereof, wherein p is 0, 1, 2, or 3.

In certain embodiments, a compound of Formula (B-I) is of Formula (B-IV):

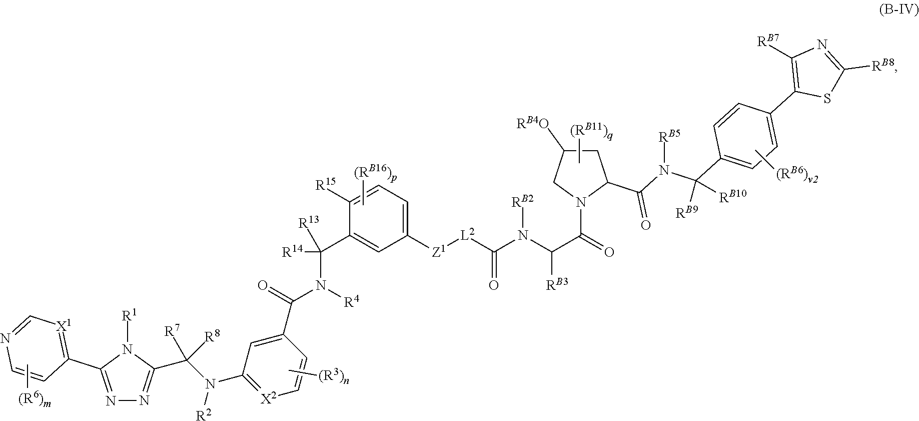

or a pharmaceutically acceptable salt, stereoisomer, or tautomer, or isotopically labeled derivative thereof, wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

In certain embodiments, a compound of Formula (B-I) is of Formula (B-V):

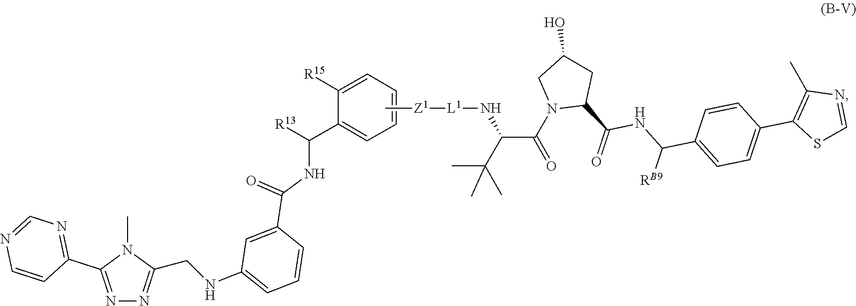

or a pharmaceutically acceptable salt, stereoisomer, tautomer, isotopically labeled derivative, solvate, hydrate, polymorph, co-crystal, or prodrug thereof.

In certain embodiments, a compound of Formula (B-I) is of Formula (B-VI):

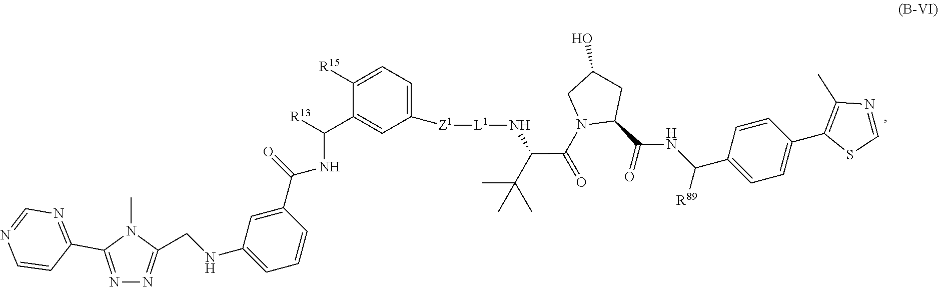

or a pharmaceutically acceptable salt, stereoisomer, tautomer, isotopically labeled derivative, solvate, hydrate, polymorph, co-crystal, or prodrug thereof.

In certain embodiments, a compound of Formula (B-I) is of Formula (B-VII):

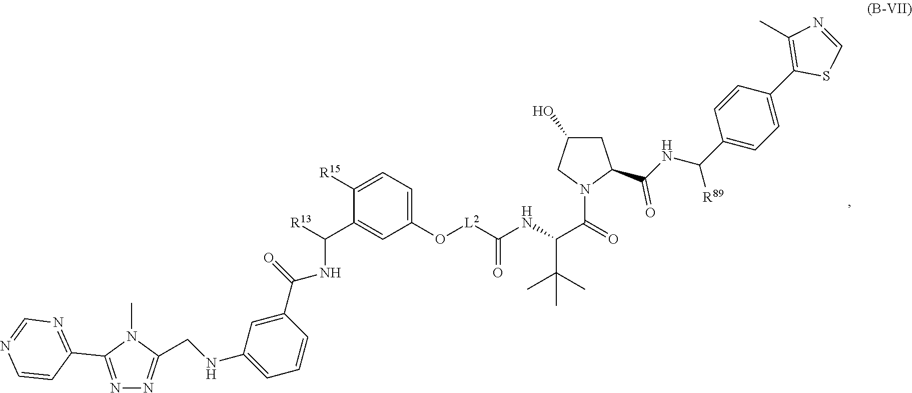

or a pharmaceutically acceptable salt, stereoisomer, tautomer, isotopically labeled derivative, solvate, hydrate, polymorph, co-crystal, or prodrug thereof.

In certain embodiments, for example, a compound of Formula (B-I) is selected from the compounds recited in Table B (infra), and pharmaceutically acceptable salts, stereoisomers, tautomers, isotopically labeled derivatives, solvates, hydrates, polymorphs, co-crystals, and prodrugs thereof.

In the various aspects and embodiments disclosed herein, express reference to a compound of Formula (B-I) is understood to alternatively refer to a compound of any disclosed subgenus or species thereof, for example, Formulae (B-II)-(B-VII), or to a compound of Table B (infra).

TABLE B
Examples of Compounds of Formula (B-I)

| Structure | Compound |
|---|---|
| 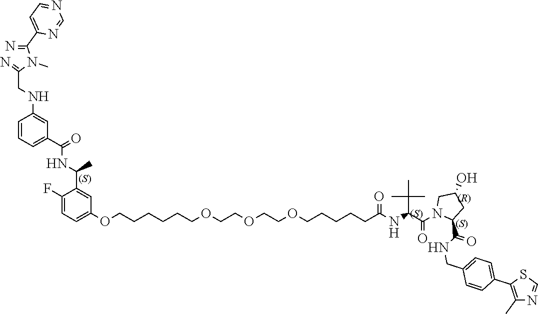 | D74 |
| 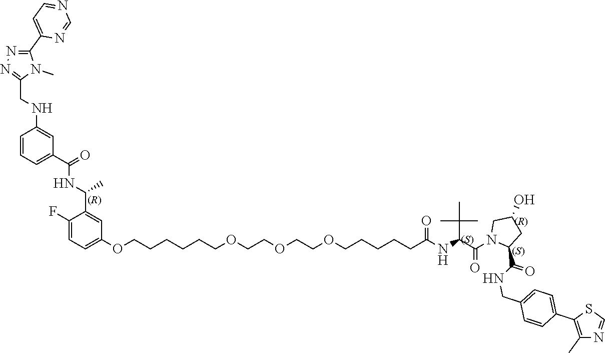 | D75 |
| 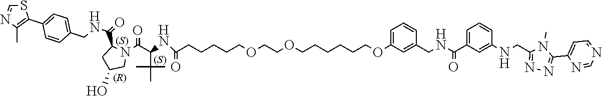 | D76 |
| 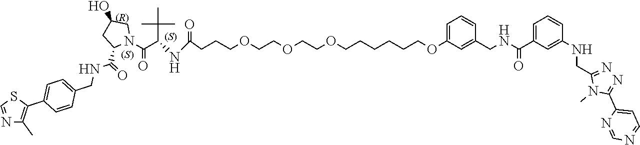 | D77 |
| 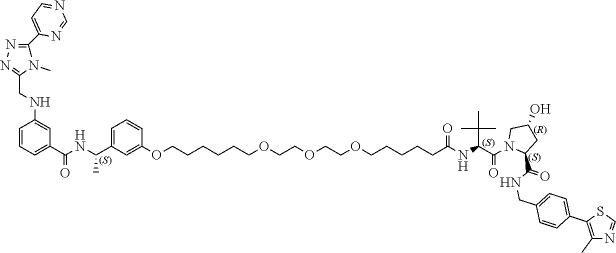 | D78 |
| 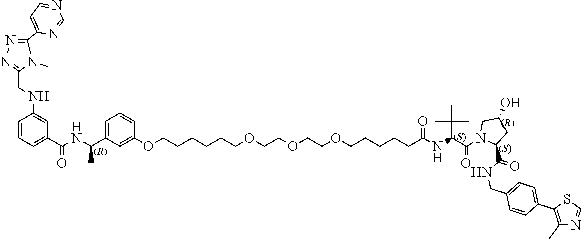 | D79 |
| 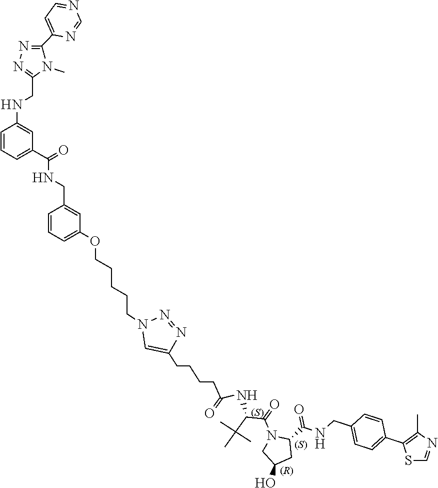 | D80 |
| 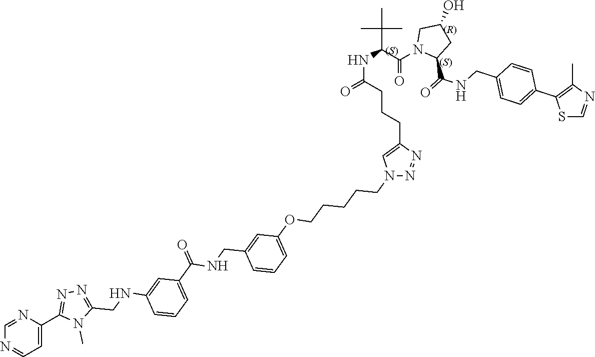 | D81 |
| 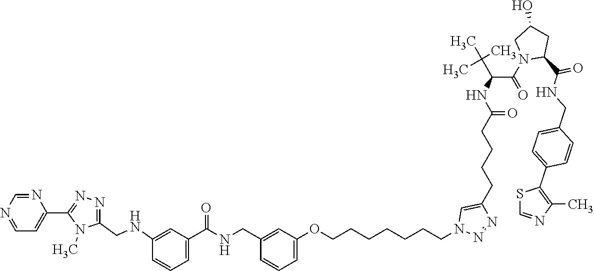 [SMILES: Cc1ncsc1-c1ccc(CNC(=O)[C@@H]2C[C@@H](O)CN2C(=O)[C@@H](NC(=O)CCCCc2cn(CCCCCCCOc3cccc(CNC(=O)c4cccc(NCc5nnc(-c6ccncn6)n5C)c4)c3)nn2)C(C)(C)C)cc1] | D82 |
| 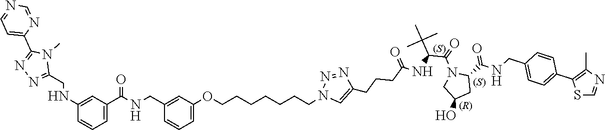 | D83 |
| 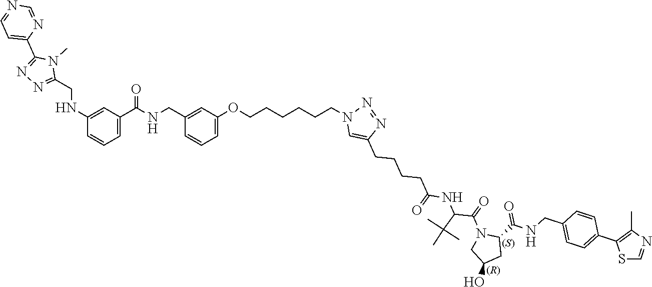 | D84 |
| 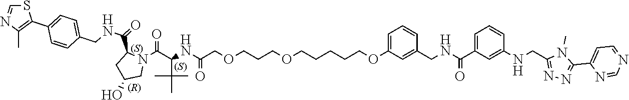 | D85 |
| 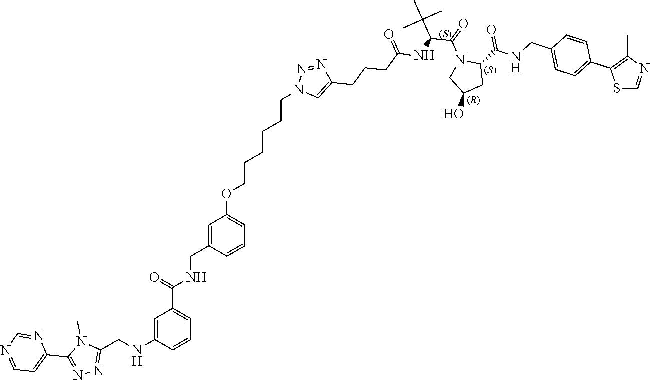 | D86 |
| 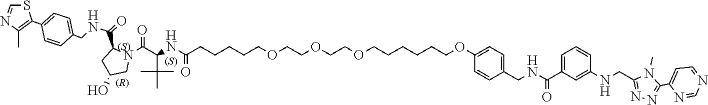 | D87 |
| 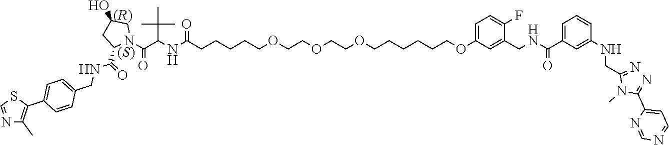 | D88 |
| 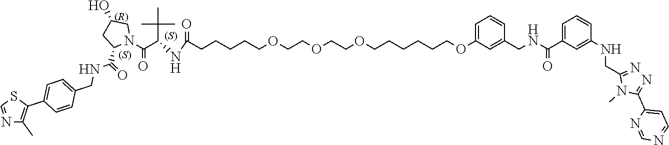 | D89 |
| 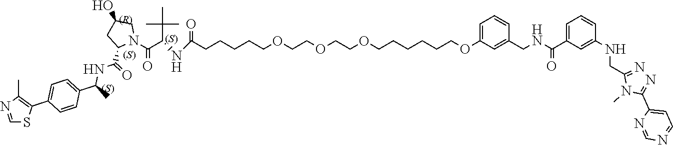 | D90 |
| 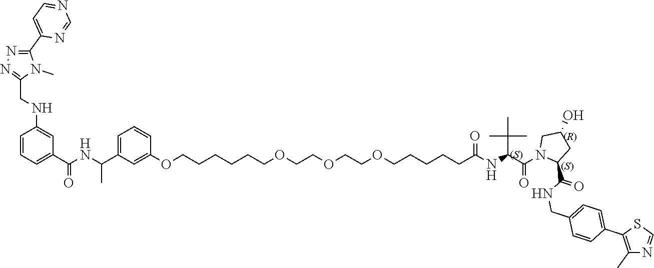 | D91 |
| 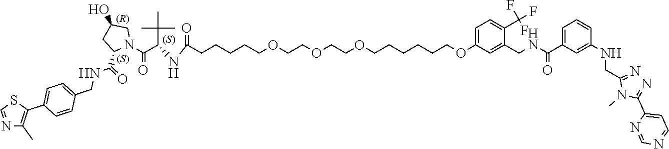 | D92 |
| 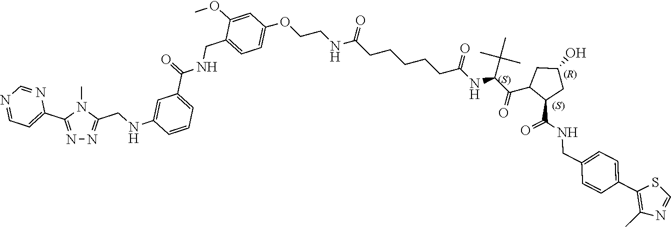 | D93 |
| 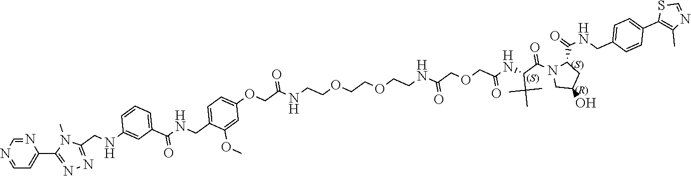 | D94 |
| 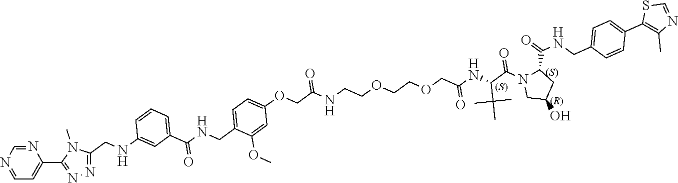 | D95 |
| 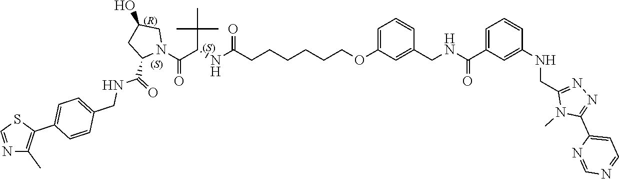 | D96 |
| 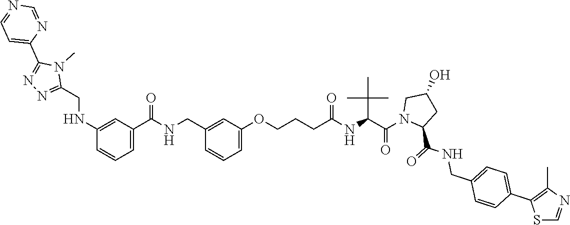 [SMILES: Cc1ncsc1-c1ccc(CNC(=O)[C@@H]2C[C@@H](O)CN2C(=O)[C@@H](NC(=O)CCCOc2cccc(CNC(=O)c3cccc(NCc4nnc(-c5ccncn5)n4C)c3)c2)C(C)(C)C)cc1] | D97 |
| 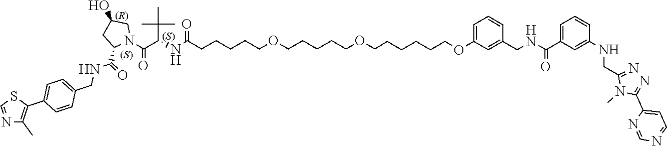 | D98 |
| 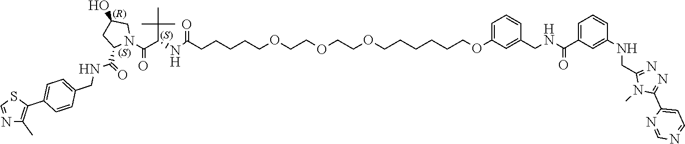 | D99 |
| 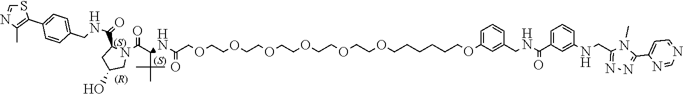 | D100 |
| 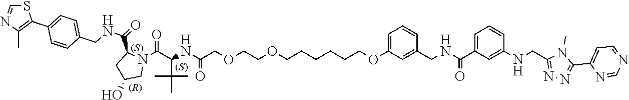 | D101 |
| 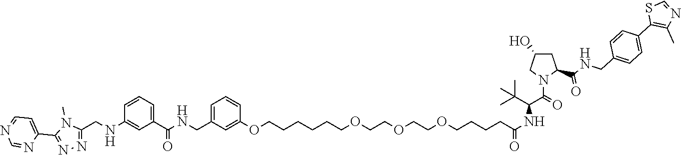 [SMILES: Cc1ncsc1-c1ccc(CNC(=O)[C@@H]2C[C@@H](O)CN2C(=O)[C@@H](NC(=O)CCCCOCCOCCOCCCCCCOc2cccc(CNC(=O)c3cccc(NCc4nnc(-c5ccncn5)n4C)c3)c2)C(C)(C)C)cc1] | D109 |
| 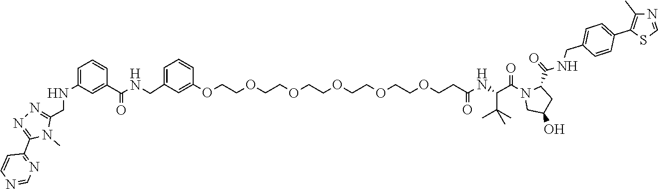 [SMILES: Cc1ncsc1-c1ccc(CNC(=O)[C@@H]2C[C@@H](O)CN2C(=O)[C@@H](NC(=O)CCOCCOCCOCCOCCOCCOc2cccc(CNC(=O)c3cccc(NCc4nnc(-c5ccncn5)n4C)c3)c2)C(C)(C)C)cc1] | D110 |
| 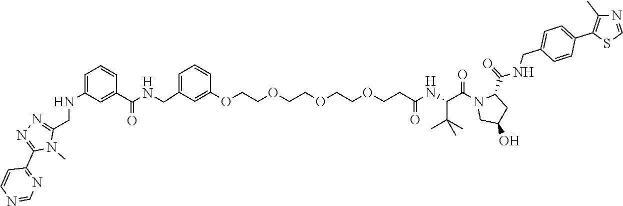 [SMILES: Cc1ncsc1-c1ccc(CNC(=O)[C@@H]2C[C@@H](O)CN2C(=O)[C@@H](NC(=O)CCOCCOCCOCCOc2cccc(CNC(=O)c3cccc(NCc4nnc(-c5ccncn5)n4C)c3)c2)C(C)(C)C)cc1] | D112 |
| 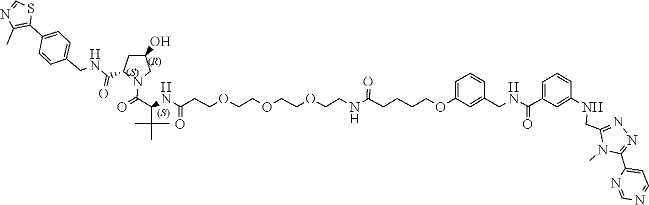 | D115 |
| 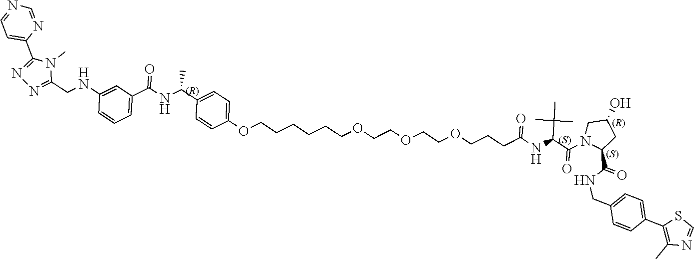 | D116 |
| 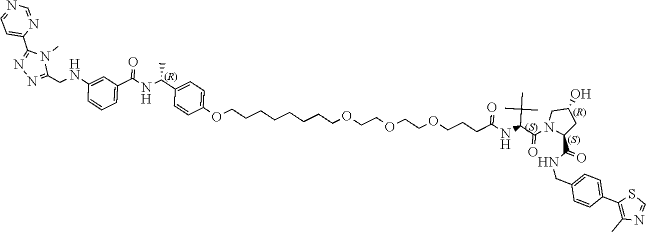 | D148 |
| 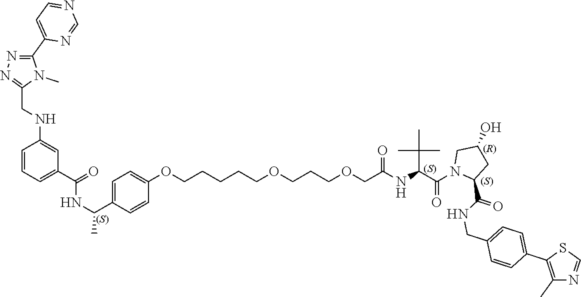 | D151 |
| 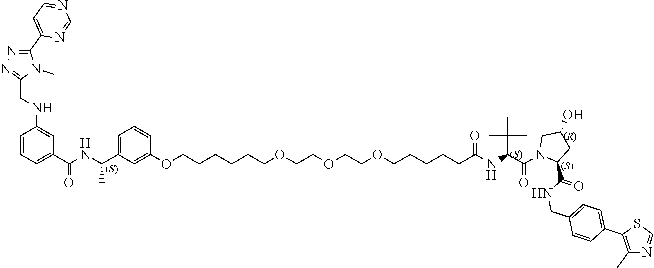 | D153 |
| 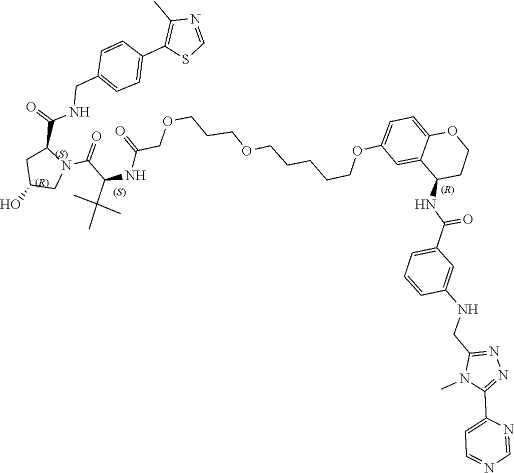 | D161 |
| 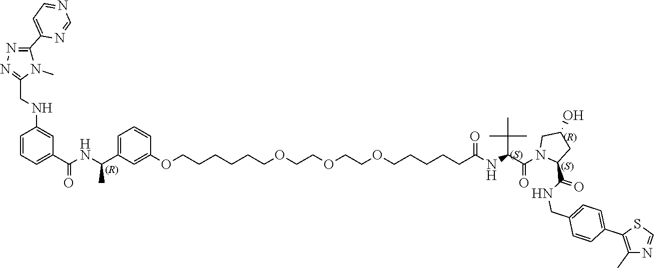 | D162 |
| 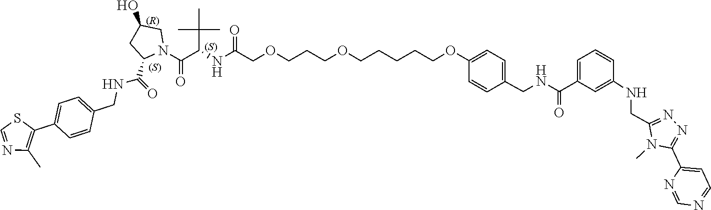 | D168 |
| 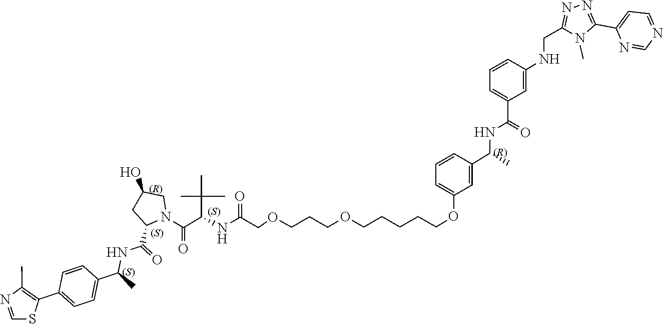 | D169 |
| 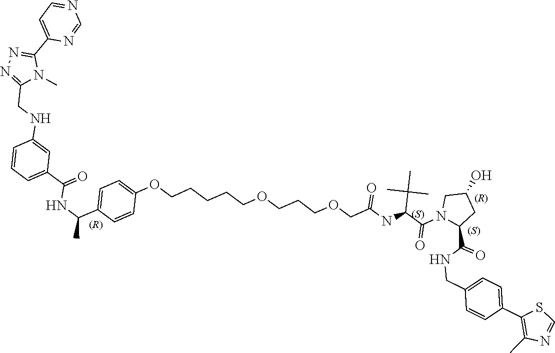 | D172 |
| 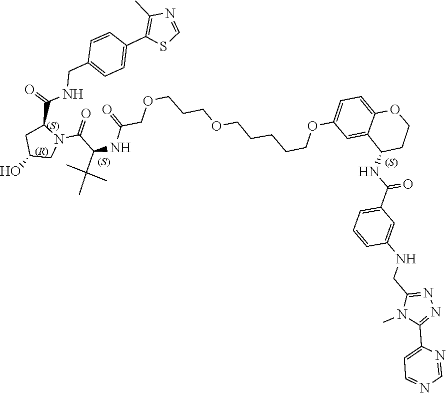 | D173 |
| 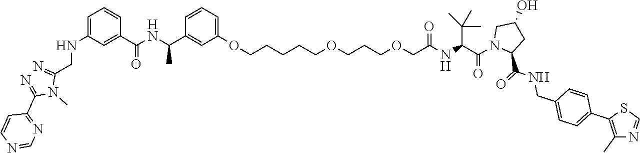 [SMILES: Cc1ncsc1-c1ccc(CNC(=O)[C@@H]2C[C@@H](O)CN2C(=O)[C@@H](NC(=O)COCCCOCCCCCOc2cccc([C@@H](C)NC(=O)c3cccc(NCc4nnc(-c5ccncn5)n4C)c3)c2)C(C)(C)C)cc1] | D182 |
| 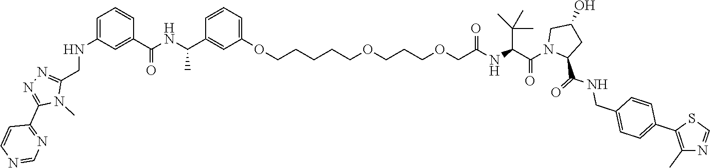 [SMILES: Cc1ncsc1-c1ccc(CNC(=O)[C@@H]2C[C@@H](O)CN2C(=O)[C@@H](NC(=O)COCCCOCCCCCOc2cccc([C@H](C)NC(=O)c3cccc(NCc4nnc(-c5ccncn5)n4C)c3)c2)C(C)(C)C)cc1] | D184 |
| 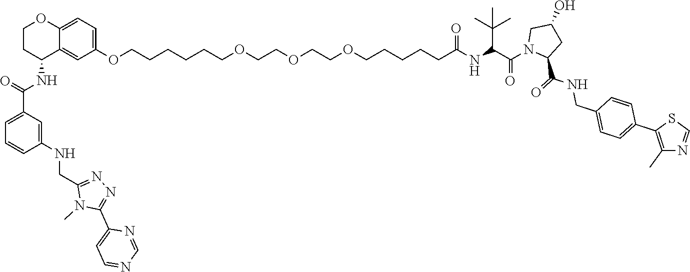 | D186 |

Also provided herein are compounds of Formula (C-I):

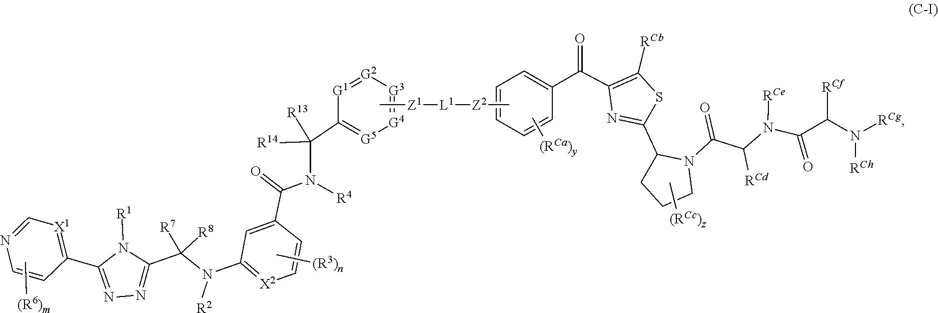

and pharmaceutically acceptable salts, stereoisomers, tautomers, isotopically labeled derivatives, solvates, hydrates, polymorphs, co-crystals, and prodrugs thereof, wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof;
Z¹ and Z² are each independently a bond, —O—, —NR^{N}, —S—, —C(═O)—, or optionally substituted —CH₂—;
G¹ is CR¹⁵ or N;
G², G³, G⁴, and G⁵ are each independently CR¹⁶, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{Ce}, R^{Cg}, and R^{Ch} are each independently hydrogen, optionally substituted C₁₋₆ alkyl, optionally substituted C₁₋₆ acyl, or a nitrogen protecting group;
optionally wherein R^{Cg} and R^{Ch} are joined together with the intervening atoms to form optionally substituted optionally substituted 3-8 membered heterocyclyl or optionally substituted 5-10 membered heteroaryl;
each instance of R³, R⁶, R⁹, R¹⁵, R¹⁶, R^{Ca}, and R^{Cb} is independently hydrogen, halogen, —CN, —N₃, —NO₂, optionally substituted C₁₋₆ alkyl, optionally substituted C₁₋₆ heteroalkyl, optionally substituted C₂₋₆alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R¹ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{Cd} and R^{Cf} are each independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, or optionally substituted C₁₋₆ acyl;
each instance of R^{Ce} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
y is 0, 1, 2, 3, or 4; and
z is 0, 1, 2, 3, 4, 5, 6, or 7.

In certain embodiments, a compound of Formula (C-I) is of Formula (C-II):

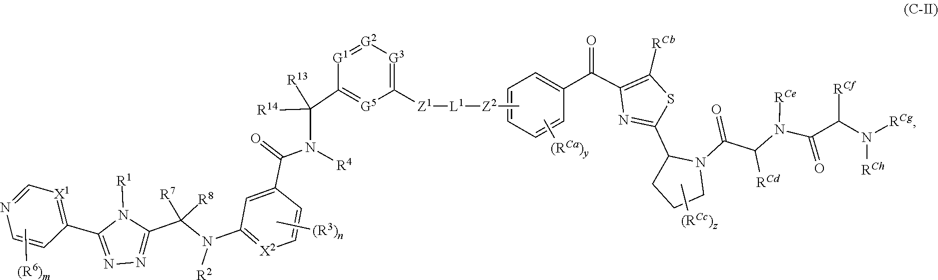

or a pharmaceutically acceptable salt, stereoisomer, tautomer, isotopically labeled derivative, solvate, hydrate, polymorph, co-crystal, or prodrug thereof.

In certain embodiments, a compound of Formula (C-I) is of Formula (C-III):

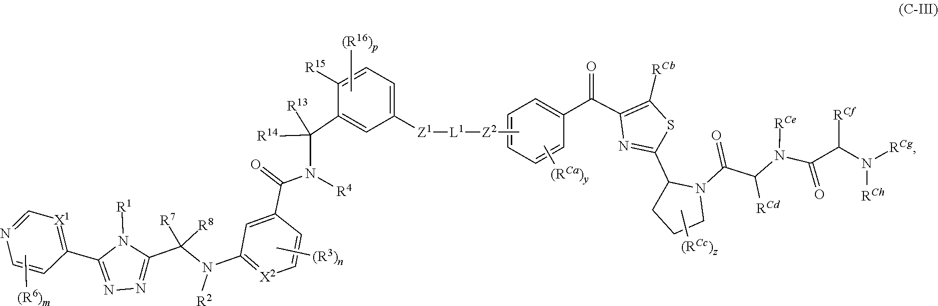

or a pharmaceutically acceptable salt, stereoisomer, tautomer, isotopically labeled derivative, solvate, hydrate, polymorph, co-crystal, or prodrug thereof, wherein p is 0, 1, 2, or 3.

In certain embodiments, a compound of Formula (C-I) is of Formula (C-IV):

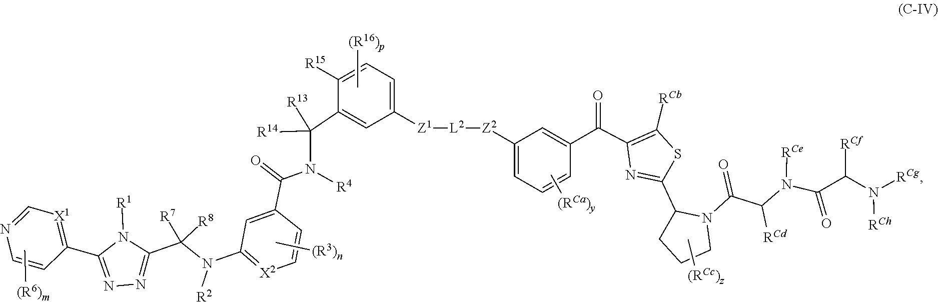

or a pharmaceutically acceptable salt, stereoisomer, tautomer, isotopically labeled derivative, solvate, hydrate, polymorph, co-crystal, or prodrug thereof, wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₅ carbocyclylene, optionally substituted 3-8 membered heterocyclylene, optionally substituted C₆-C₁₀ arylene, or optionally substituted 5-10 membered heteroarylene.

In certain embodiments, a compound of Formula (C-I) is of Formula (C-V):

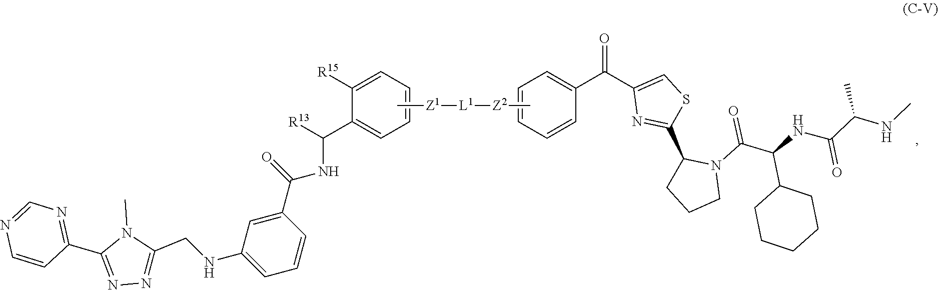

or a pharmaceutically acceptable salt, stereoisomer, tautomer, isotopically labeled derivative, solvate, hydrate, polymorph, co-crystal, or prodrug thereof.

In certain embodiments, a compound of Formula (C-I) is of Formula (C-VI):

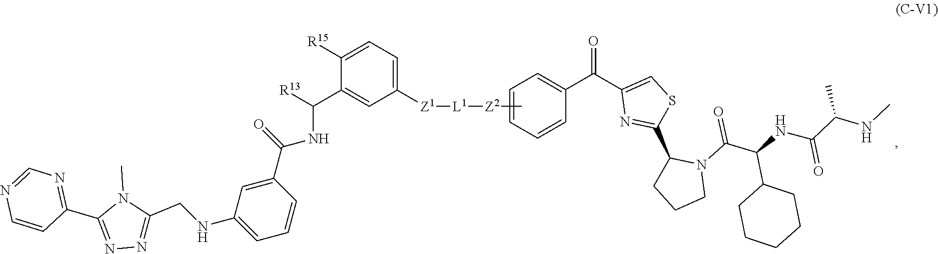

or a pharmaceutically acceptable salt, stereoisomer, tautomer, isotopically labeled derivative, solvate, hydrate, polymorph, co-crystal, or prodrug thereof.

In certain embodiments, a compound of Formula (C-I) is of Formula (C-VII):

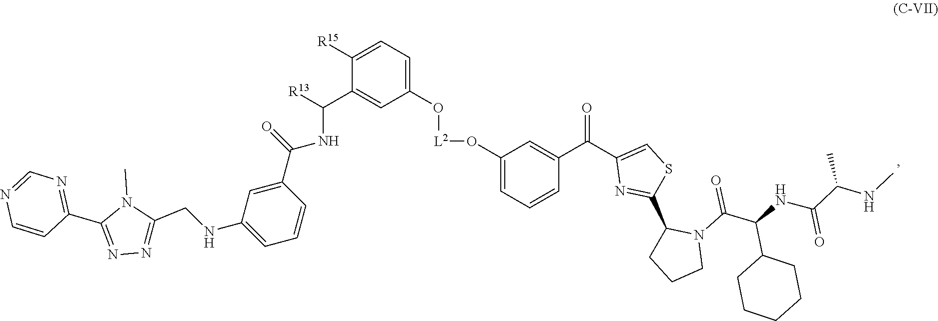

or a pharmaceutically acceptable salt, stereoisomer, tautomer, isotopically labeled derivative, solvate, hydrate, polymorph, co-crystal, or prodrug thereof.

In certain embodiments, for example, a compound of Formula (C-I) is selected from the compounds recited in Table C (infra), and pharmaceutically acceptable salts, stereoisomers, tautomers, isotopically labeled derivatives, solvates, hydrates, polymorphs, co-crystals, and prodrugs thereof.

In the various aspects and embodiments disclosed herein, express reference to a compound of Formula (C-I) is understood to alternatively refer to a compound of any disclosed subgenus or species thereof, for example, Formulae (C-II)-(C-VII), or to a compound of Table C (infra).

TABLE C
Examples of Compounds of Formula (C-I)

| Structure | Compound |
|---|---|
| 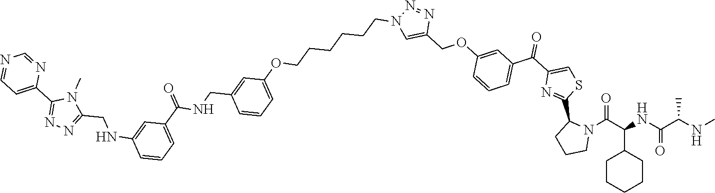 [SMILES: CN[C@@H](C)C(=O)N[C@H](C(=O)N1CCC[C@H]1c1nc(C(=O)c2cccc(OCc3cn(CCCCCCOc4cccc(CNC(=O)c5cccc(NCc6nnc(-c7ccncn7)n6C)c5)c4)nn3)c2)cs1)C1CCCCC1] | D102 |
| 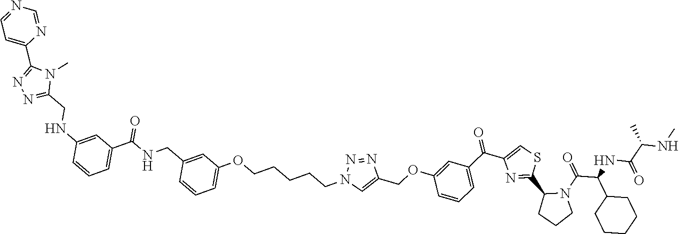 [SMILES: CN[C@@H](C)C(=O)N[C@H](C(=O)N1CCC[C@H]1c1nc(C(=O)c2cccc(OCc3cn(CCCCCOc4cccc(CNC(=O)c5cccc(NCc6nnc(-c7ccncn7)n6C)c5)c4)nn3)c2)cs1)C1CCCCC1] | D103 |
| 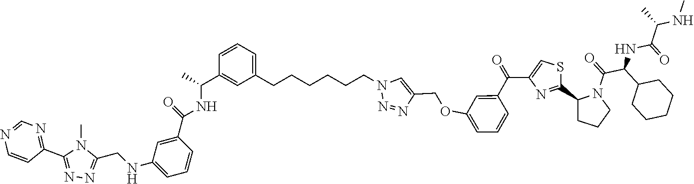 [SMILES: CN[C@@H](C)C(=O)N[C@H](C(=O)N1CCC[C@H]1c1nc(C(=O)c2cccc(OCc3cn(CCCCCCc4cccc([C@@H](C)NC(=O)c5cccc(NCc6nnc(-c7ccncn7)n6C)c5)c4)nn3)c2)cs1)C1CCCCC1] | D104 |
| 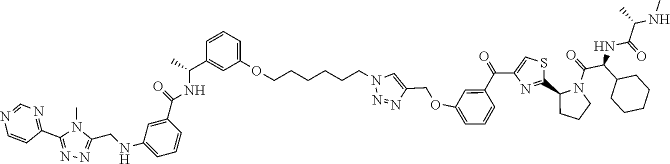 [SMILES: CN[C@@H](C)C(=O)N[C@H](C(=O)N1CCC[C@H]1c1nc(C(=O)c2cccc(OCc3cn(CCCCCCOc4cccc([C@@H](C)NC(=O)c5cccc(NCc6nnc(-c7ccncn7)n6C)c5)c4)nn3)c2)cs1)C1CCCCC1] | D105 |
| 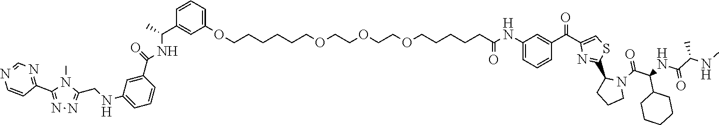 [SMILES: CN[C@@H](C)C(=O)N[C@H](C(=O)N1CCC[C@H]1c1nc(C(=O)c2cccc(NC(=O)CCCCCOCCOCCOCCCCCCOc3cccc([C@@H](C)NC(=O)c4cccc(NCc5nnc(-c6ccncn6)n5C)c4)c3)c2)cs1)C1CCCCC1] | D106 |
| 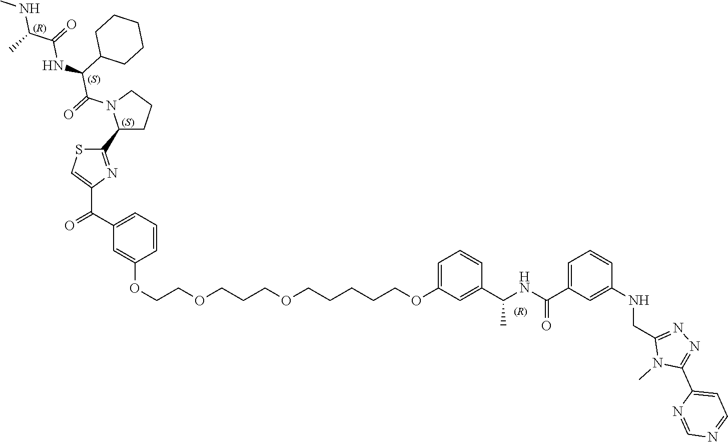 | D149 |

The following definitions and embodiments apply to all generic formulae comprising the relevant groups (e.g., any of Formulae (A-I)-(A-VII), Formulae (B-I)-(B-VII), Formulae (C-I)-(C-VII), etc.) provided herein.

In certain embodiments, L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof.

In certain embodiments, L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof.

In certain embodiments, L¹ is optionally substituted C₁₋₄₀ alkylene. In certain embodiments, L¹ is optionally substituted C₁₋₄₀ heteroalkylene. In certain embodiments, L¹ is optionally substituted C₁₋₃₀ alkylene. In certain embodiments, L¹ is optionally substituted C₁₋₃₀ heteroalkylene. In certain embodiments, L¹ is optionally substituted C₁₋₂₀ alkylene. In certain embodiments, L¹ is optionally substituted C₁₋₂₀ heteroalkylene. In certain embodiments, the alkylene or heteroalkylene of L¹ is substituted with one or more (e.g., 1, 2, 3, 4, 5, 6, 7, 8, 9, 10) instances of ═O. In certain embodiments, L¹ is -L²-C(C═O)—. In certain embodiments, the heteroalkylene comprises one or more (e.g., 1, 2, 3, 4, 5, 6, 7, 8, 9, 10) heteroatoms independently selected from of O and N. In certain embodiments, the heteroalkylene comprises one or more (e.g., 1, 2, 3, 4, 5, 6, 7, 8, 9, 10) groups independently selected from —C(═O)—, —O—, —NR^{N}—, —OC(═O)—, —C(═O)O—, —NR^{N}C(═O)—, and —C(═O)NR^{N}—. In certain embodiments, the heteroalkylene comprises one or more (e.g., 1, 2, 3, 4, 5, 6, 7, 8, 9, 10) groups independently selected from —C(═O)—, —O—, —NH—, —OC(═O)—, —C(═O)O—, —NHC(═O)—, and —C(═O)NH—.

In certain embodiments, L¹ is unsubstituted C₁₋₄₀ alkylene. In certain embodiments, L¹ is unsubstituted C₁₋₄₀ heteroalkylene. In certain embodiments, L¹ is unsubstituted C₁₋₃₀ alkylene. In certain embodiments, L¹ is unsubstituted C₁₋₃₀ heteroalkylene. In certain embodiments, L¹ is unsubstituted C₁₋₂₀ alkylene. In certain embodiments, L¹ is unsubstituted C₁₋₂₀ heteroalkylene. In certain embodiments, the heteroalkylene of L¹ comprises one or more (e.g., 1, 2, 3, 4, 5, 6, 7, 8, 9, 10) heteroatoms independently selected from O and N. In certain embodiments, the heteroalkylene comprises one or more (e.g., 1, 2, 3, 4, 5, 6, 7, 8, 9, 10) groups independently selected from —O— and —NH—.

In certain embodiments, the alkylene or heteroalkylene of L¹ is interrupted with at least one instance of optionally substituted triazolylene. In certain embodiments, the alkylene or heteroalkylene of L¹ is interrupted with at least one instance of:

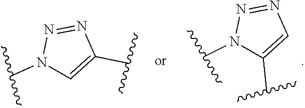 [SMILES: *c1cn(*)nn1 ; *c1cnnn1*]

In certain embodiments, the alkylene or heteroalkylene of L¹ is interrupted with at least one instance (e.g., 1 or 2 instances) of optionally substituted heterocyclylene. In certain embodiments, the alkylene or heteroalkylene of L¹ is interrupted with at least one instance (e.g., 1 or 2 instances) of optionally substituted 4-6 membered heterocyclylene. In certain embodiments, the alkylene or heteroalkylene of L¹ is interrupted with at least one instance (e.g., 1 or 2 instances) of optionally substituted 4-6 membered heterocyclylene comprising 1 or 2 heteroatoms selected from N and O. In certain embodiments, the alkylene or heteroalkylene of L¹ is interrupted with at least one instance (e.g., 1 or 2 instances) of optionally substituted 4-6 membered heterocyclylene comprising 1 or 2 N atoms.

In certain embodiments, the alkylene or heteroalkylene of L¹ is interrupted with at least one instance (e.g., 1 or 2 instances) of unsubstituted heterocyclylene. In certain embodiments, the alkylene or heteroalkylene of L¹ is interrupted with at least one instance (e.g., 1 or 2 instances) of unsubstituted 4-6 membered heterocyclylene. In certain embodiments, the alkylene or heteroalkylene of L¹ is interrupted with at least one instance (e.g., 1 or 2 instances) of unsubstituted 4-6 membered heterocyclylene comprising 1 or 2 heteroatoms selected from N and O. In certain embodiments, the alkylene or heteroalkylene of L¹ is interrupted with at least one instance (e.g., 1 or 2 instances) of unsubstituted 4-6 membered heterocyclylene comprising 1 or 2 N atoms. In certain embodiments, the alkylene or heteroalkylene of L¹ is interrupted with at least one instance (e.g., 1 or 2 instances) of:

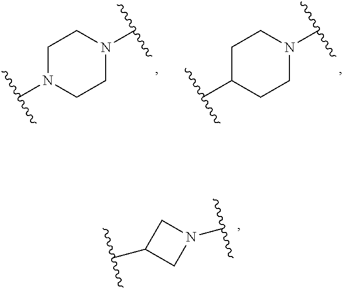 [SMILES: *N1CCN(*)CC1 ; *C1CCN(*)CC1 ; *C1CN(*)C1]

or a combination thereof.

As used herein, when a group (i.e., L¹) is “interrupted” with a moiety, said moiety may appear between two atoms of the group (i.e., internally) or on the terminus of the group. For example, in an instance where L¹ is interrupted with

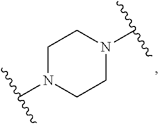 [SMILES: *N1CCN(*)CC1]

L¹ may be of the formula:

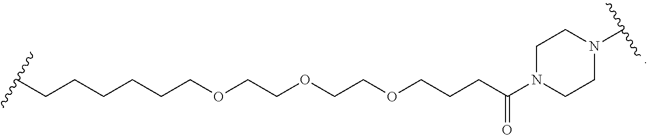 [SMILES: *CCCCCCOCCOCCOCCCC(=O)N1CCN(*)CC1]

In certain embodiments, for example, L¹ is of one of the following formulae:

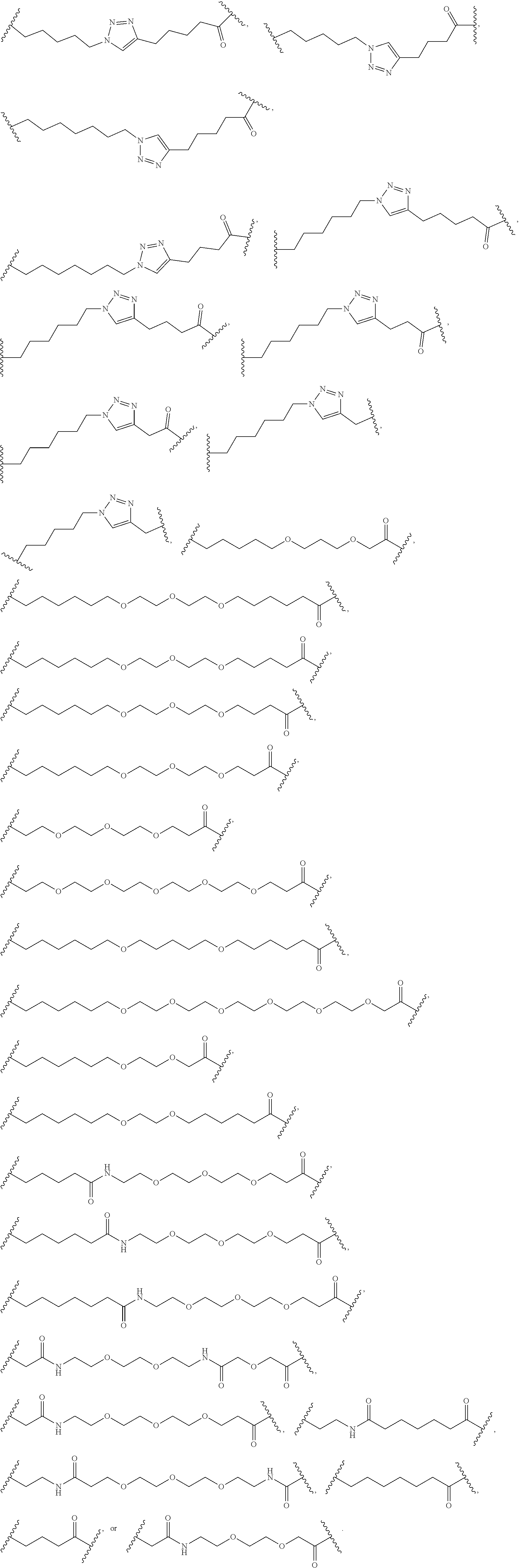

In certain embodiments, L¹ is of one of the following formulae:

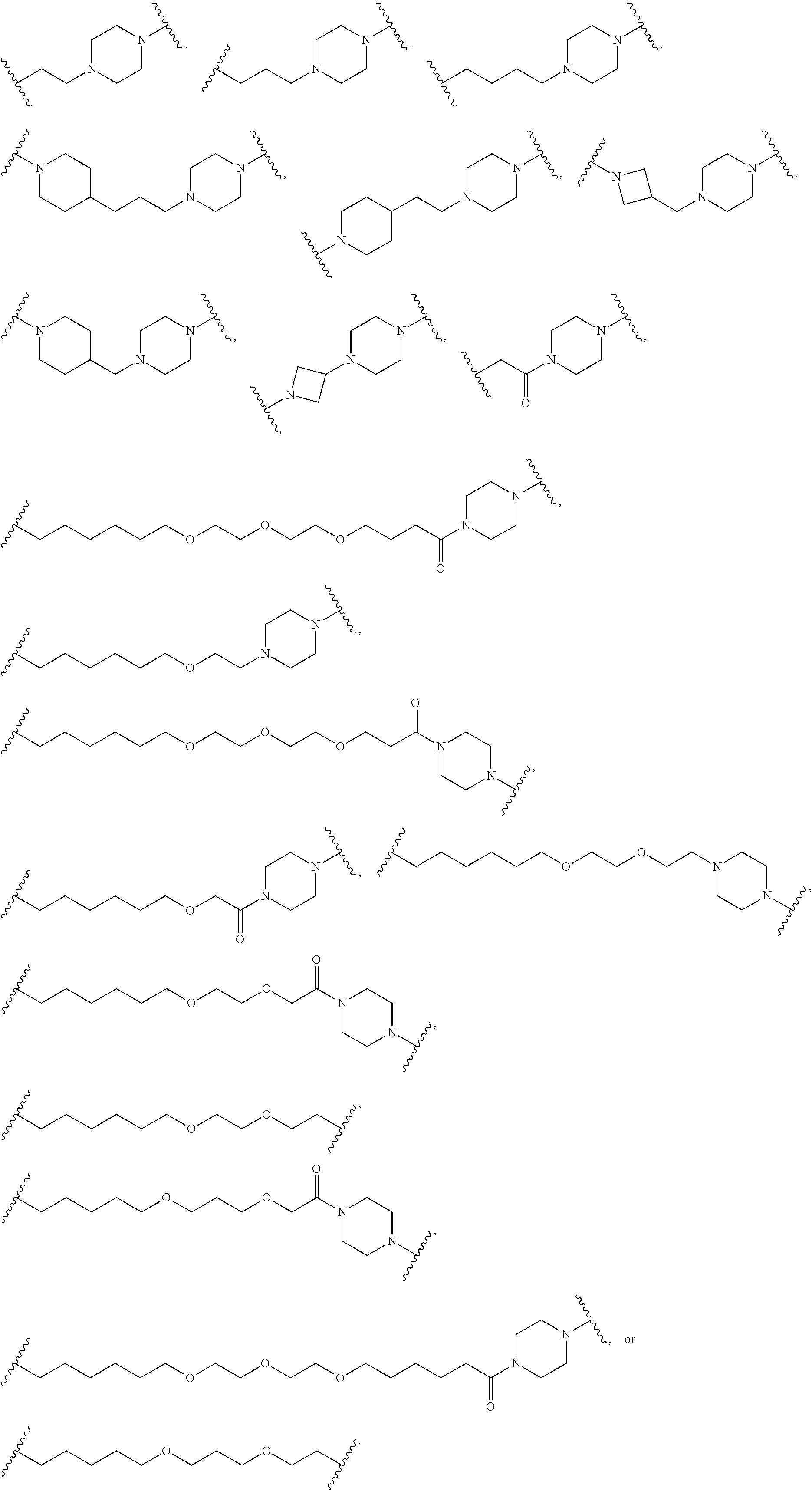

In certain embodiments, for example, L¹ is of one of the following formulae:

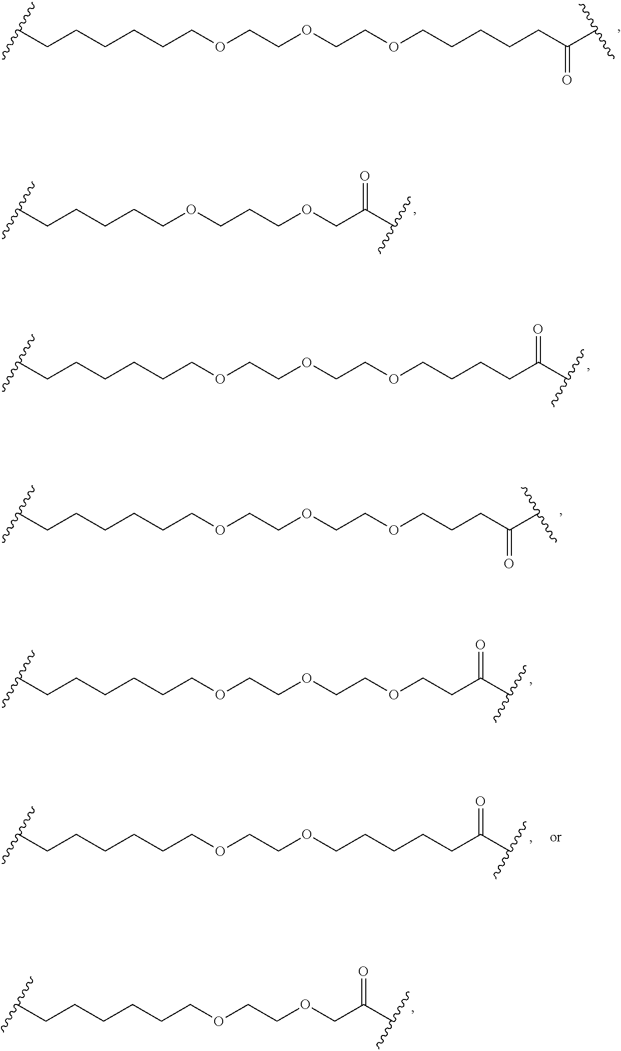

In certain embodiments, Z¹ is a bond, —O—, —NR^{N}—, —S—, —C(═O)—, or optionally substituted —CH₂—. In certain embodiments, Z¹ is a bond, —O—, —NH—, —S—, or —CH₂—. In certain embodiments, Z¹ is —O—. In certain embodiments, Z¹ is a bond. In certain embodiments, Z¹ is —C(═O)—. In certain embodiments, Z¹ is —NR^{N}—. In certain embodiments, Z¹ is —NH—.

In certain embodiments, Z² is a bond, —O—, —NR^{A11}, —S—, —C(═O)—, or optionally substituted —CH₂—. In certain embodiments, Z² is a bond, —O—, —NR^{N}—, —S—, or optionally substituted —CH₂—. In certain embodiments, Z² is a bond, —O—, —NH—, —S—, or —CH₂—. In certain embodiments, Z² is —NR^{A11}. In certain embodiments, Z² is —NR^{N}—. In certain embodiments, Z² is —NH—. In certain embodiments, Z² is —O—. In certain embodiments, Z² is a bond. In certain embodiments, Z² is —C(═O)—.

In certain embodiments, L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

In certain embodiments, L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally substituted C₃-C₈ carbocyclylene, optionally substituted 3-8 membered heterocyclylene, optionally substituted C₆-C₁₀ arylene, optionally substituted 5-10 membered heteroarylene, and any combination thereof.

In certain embodiments, L² is optionally substituted C₁₋₃₀ alkylene. In certain embodiments, L² is optionally substituted C₁₋₃₀ heteroalkylene. In certain embodiments, L² is optionally substituted C₁₋₂₀ alkylene. In certain embodiments, L² is optionally substituted C₁₋₂₀ heteroalkylene. In certain embodiments, L² is optionally substituted C₁₋₁₀ alkylene. In certain embodiments, L² is optionally substituted C₁₋₁₀ heteroalkylene. In certain embodiments, the alkylene or heteroalkylene of L² is substituted with one or more (e.g., 1, 2, 3, 4, 5, 6, 7, 8, 9, 10) instances of ═O. In certain embodiments, the heteroalkylene comprises one or more (e.g., 1, 2, 3, 4, 5, 6, 7, 8, 9, 10) heteroatoms independently selected from of O and N. In certain embodiments, the heteroalkylene comprises one or more (e.g., 1, 2, 3, 4, 5, 6, 7, 8, 9, 10) groups independently selected from —C(═O)—, —O—, —NR^{N}—, —OC(═O)—, —C(═O)O—, —NR^{N}C(═O)—, and —C(═O)NR^{N}—. In certain embodiments, the heteroalkylene comprises one or more (e.g., 1, 2, 3, 4, 5, 6, 7, 8, 9, 10) groups independently selected from —C(═O)—, —O—, —NH—, —OC(═O)—, —C(═O)O—, —NHC(═O)—, and —C(═O)NH—.

In certain embodiments, L² is unsubstituted C₁₋₃₀ alkylene. In certain embodiments, L² is unsubstituted C₁₋₃₀ heteroalkylene. In certain embodiments, L² is unsubstituted C₁₋₂₀ alkylene. In certain embodiments, L² is unsubstituted C₁₋₂₀ heteroalkylene. In certain embodiments, L² is unsubstituted C₁₋₁₀ alkylene. In certain embodiments, L² is unsubstituted C₁₋₁₀ heteroalkylene. In certain embodiments, the heteroalkylene comprises one or more (e.g., 1, 2, 3, 4, 5, 6, 7, 8, 9, 10) heteroatoms independently selected from O and N. In certain embodiments, the heteroalkylene comprises one or more (e.g., 1, 2, 3, 4, 5, 6, 7, 8, 9, 10) groups independently selected from —O— and —NH—.

In certain embodiments, the alkylene or heteroalkylene of L² is interrupted with at least one instance of optionally substituted triazolylene. In certain embodiments, the alkylene or heteroalkylene of L² is interrupted with at least one instance of:

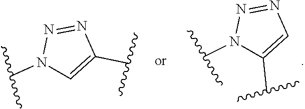 [SMILES: *c1cn(*)nn1 ; *c1cnnn1*]

In certain embodiments, L² is of one of the following formulae:

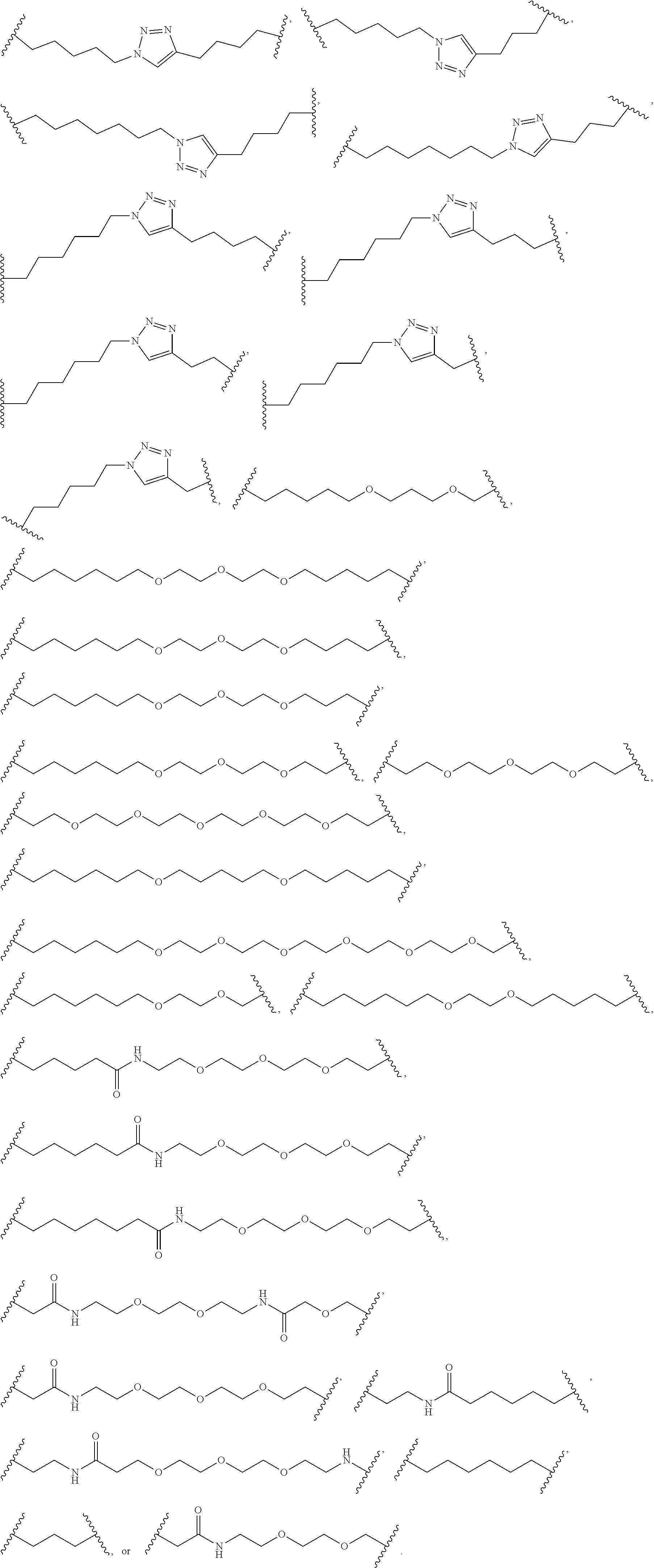

In certain embodiments, L² is of one of the following formulae:

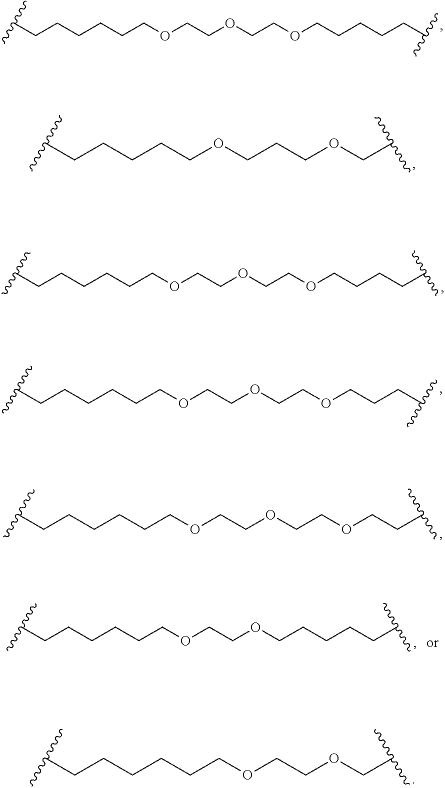

In certain embodiments, G¹ is CR⁵ or N. In certain embodiments, G¹ is CR¹⁵.

In certain embodiments, G² is CR¹⁶, CH, or N. In certain embodiments, G² is CR¹⁶. In certain embodiments, G² is CH.

In certain embodiments, G³ is CR¹⁶, CH, or N. In certain embodiments, G³ is CR¹⁶. In certain embodiments, G³ is CH.

In certain embodiments, G⁴ is CR¹⁶, CH, or N. In certain embodiments, G⁴ is CR¹⁶. In certain embodiments, G⁴ is CH.

In certain embodiments, G⁵ is CR¹⁶, CH, or N. In certain embodiments, G⁵ is CR¹⁶. In certain embodiments, G⁵ is CH.

In certain embodiments, G², G³, G⁴, and G⁵ are independently CR¹⁶ or CH. In certain embodiments, G², G³, G⁴, and G⁵ are CR¹⁶. In certain embodiments, G², G³, G⁴, and G⁵ are CH.

In certain embodiments, X¹ is CR⁹ or N. In certain embodiments, X¹ is N.

In certain embodiments, X² is CR³ or N. In certain embodiments, X² is CR³. In certain embodiments, X² is CH.

In certain embodiments, R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group.

In certain embodiments, R¹ is optionally substituted C₁₋₆ alkyl. In certain embodiments, R¹ is unsubstituted C₁₋₆ alkyl. In certain embodiments, R¹ is unsubstituted C₁₋₃ alkyl. In certain embodiments, R¹ is methyl. In certain embodiments, R¹ is hydrogen.

In certain embodiments, R¹ is C₁₋₆ alkyl substituted with —OR^{O}. In certain embodiments, R¹ is C₁₋₆ alkyl substituted with —OH.

In certain embodiments, R² is hydrogen, optionally substituted C₁₋₆ alkyl, optionally substituted C₁₋₆ acyl, or a nitrogen protecting group. In certain embodiments, R² is hydrogen. In certain embodiments, R² is optionally substituted C₁₋₆ alkyl.

In certain embodiments, each instance of R³ is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R³ is hydrogen. In certain embodiments, each instance of R³ is hydrogen. In certain embodiments, at least one instance of R³ is optionally substituted C₁₋₆ alkyl. In certain embodiments, at least one instance of R³ is halogen.

In certain embodiments, R⁴ is hydrogen, optionally substituted C₁₋₆ alkyl, optionally substituted C₁₋₆ acyl, or a nitrogen protecting group. In certain embodiments, R⁴ is hydrogen. In certain embodiments, R⁴ is optionally substituted C₁₋₆ alkyl.

In certain embodiments, each instance of R⁶ is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R⁶ is hydrogen. In certain embodiments, at least one instance of R⁶ is optionally substituted C₁₋₆ alkyl. In certain embodiments, at least one instance of R⁶ is halogen.

In certain embodiments, R⁷ is hydrogen, deuterium, or optionally substituted C₁₋₆ alkyl. In certain embodiments, R⁷ is hydrogen. In certain embodiments, R⁷ is optionally substituted C₁₋₆ alkyl.

In certain embodiments, R¹ is hydrogen, deuterium, or optionally substituted C₁₋₆ alkyl. In certain embodiments, R⁷ is hydrogen. In certain embodiments, R⁷ is optionally substituted C₁₋₆ alkyl.

In certain embodiments, R⁷ and R⁸ are hydrogen.

In certain embodiments, R⁷ is deuterium. In certain embodiments, R⁸ is deuterium. In certain embodiments, R⁷ and R⁸ are deuterium.

In certain embodiments, R⁹ is hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, R⁹ is hydrogen. In certain embodiments, R⁹ is optionally substituted C₁₋₆ alkyl. In certain embodiments, R⁹ is halogen.

In certain embodiments, R⁹ and R¹ are joined together with the intervening atoms to form optionally substituted 4-8 membered heterocyclyl.

In certain embodiments, R¹³ is hydrogen or optionally substituted C₁₋₆ alkyl. In certain embodiments, R¹³ is optionally substituted C₁₋₆ alkyl. In certain embodiments, R¹³ is unsubstituted C₁₋₆ alkyl. In certain embodiments, R¹³ is unsubstituted C₁₋₃ alkyl. In certain embodiments, R¹³ is methyl. In certain embodiments, R¹³ is hydrogen.

In certain embodiments, R¹⁴ is hydrogen or optionally substituted C₁₋₆ alkyl. In certain embodiments, R¹⁴ is hydrogen. In certain embodiments, R¹⁴ is optionally substituted C₁₋₆ alkyl.

In certain embodiments, R¹³ and R¹⁴ are hydrogen. In certain embodiments, R¹³ is optionally substituted C₁₋₆ alkyl; and R¹⁴ is hydrogen. In certain embodiments, R¹³ is unsubstituted C₁₋₆ alkyl; and R¹⁴ is hydrogen. In certain embodiments, R¹³ is unsubstituted C₁₋₃ alkyl; and R¹⁴ is hydrogen. In certain embodiments, R¹³ is methyl; and R¹⁴ is hydrogen.

In certain embodiments, R¹³ and R¹⁴ are joined together with the intervening atoms to form optionally substituted C₃₋₈ carbocyclyl or 3-8 membered heterocyclyl.

In certain embodiments, R¹⁵ is hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, R¹⁵ is optionally substituted C₁₋₆ alkyl. In certain embodiments, R¹⁵ is halogen.

In certain embodiments, R¹⁵ is halogen. In certain embodiments, R¹⁵ is —F.

In certain embodiments, R¹⁵ is C₁₋₆ haloalkyl. In certain embodiments, R¹⁵ is C₁₋₃ haloalkyl. In certain embodiments, R¹⁵ is trihalomethyl. In certain embodiment, R¹⁵ is —CF₃.

In certain embodiments, R¹⁵ is —O—R^{O}. In certain embodiments, R¹⁵ is —O—C₁₋₆ alkyl. In certain embodiments, R¹⁵ is —O—C₁₋₃ alkyl. In certain embodiments, R¹⁵ is —OMe.

In certain embodiments, R¹³ and R¹⁵ are joined together with the intervening atoms to form optionally substituted C₄₋₈ carbocyclyl or optionally substituted 4-8 membered heterocyclyl.

In certain embodiments, R¹³ and R¹⁵ are joined together with the intervening atoms to form optionally substituted 5-7 membered heterocyclyl. In certain embodiments, R¹³ and R¹⁵ are joined together with the intervening atoms to form optionally substituted 6-membered heterocyclyl. In certain embodiments, R¹³ and R¹⁵ are joined together to form:

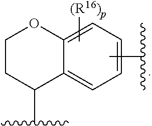

In certain embodiments, R¹³ and R¹⁵ are joined together to form:

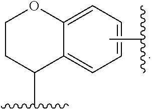

In certain embodiments, each instance of R¹⁶ is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R¹⁶ is hydrogen. In certain embodiments, at least one instance of R¹⁶ is optionally substituted C₁₋₆ alkyl. In certain embodiments, at least one instance of R¹⁶ is halogen.

In certain embodiments, m is 0, 1, 2, or 3. In certain embodiments, m is 0.

In certain embodiments, n is 0, 1, 2, or 3. In certain embodiments, n is 0.

In certain embodiments, p is 0, 1, 2, or 3. In certain embodiments, p is 0.

In certain embodiments, Q¹ is CR^{A1}, CH, or N. In certain embodiments, Q¹ is CR^{A1}. In certain embodiments, Q¹ is CH.

In certain embodiments, Q² is CR^{A1}, CH, or N. In certain embodiments, Q² is CR^{A1}. In certain embodiments, Q² is CH.

In certain embodiments, Q³ is CR^{A1}, CH, or N. In certain embodiments, Q³ is CR^{A1}. In certain embodiments, Q³ is CH.

In certain embodiments, Q⁴ is CR^{A1}, CH, or N. In certain embodiments, Q⁴ is CR^{A1}. In certain embodiments, Q⁴ is CH.

In certain embodiments, Q¹, Q², Q³, and Q⁴ are independently CR^{A1} or CH. In certain embodiments, Q¹, Q², Q³, and Q⁴ are independently CR^{A1}. In certain embodiments, Q¹, Q², Q³, and Q⁴ are CH.

In certain embodiments, each instance of R^{A1} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R^{A1} is hydrogen. In certain embodiments, at least one instance of R^{A1} is optionally substituted C₁₋₆ alkyl. In certain embodiments, at least one instance of R^{A1} is halogen.

In certain embodiments, R^{A5} is hydrogen, optionally substituted C₁₋₆ alkyl, optionally substituted C₁₋₆ acyl, or a nitrogen protecting group. In certain embodiments, R¹ is hydrogen. In certain embodiments, R^{A5} is optionally substituted C₁₋₆alkyl.

In certain embodiments, R^{A6} is hydrogen or optionally substituted C₁₋₆ alkyl. In certain embodiments, R^{A6} is hydrogen. In certain embodiments, R^{A6} is optionally substituted C₁₋₆ alkyl.

In certain embodiments, R^{A7} is hydrogen or optionally substituted C₁₋₆ alkyl. In certain embodiments, R^{A7} is hydrogen. In certain embodiments, R^{A7} is optionally substituted C₁₋₆ alkyl.

In certain embodiments, R^{A6} and R^{A7} are joined together with the intervening atoms to form optionally substituted C₃₋₈ carbocyclyl or 3-8 membered heterocyclyl.

In certain embodiments, R^{A6} and R^{A7} are taken together to form ═O.

In certain embodiments, each instance of R^{A10} is independently halogen or optionally substituted C₁-C₆ alkyl. In certain embodiments, at least one instance of R^{A10} is optionally substituted C₁₋₆ alkyl. In certain embodiments, at least one instance of R^{A10} is halogen.

In certain embodiments, R^{A11} is hydrogen, optionally substituted C₁₋₆ alkyl, optionally substituted C₁₋₆ acyl, or a nitrogen protecting group. In certain embodiments, R^{A11} is hydrogen. In certain embodiments, R^{A11} is optionally substituted C₁₋₆ alkyl.

In certain embodiments, s is 0, 1, 2, or 3. In certain embodiments, s is 0.

In certain embodiments, r is 0, 1, 2, 3, 4, or 5. In certain embodiments, r is 0.

In certain embodiments, R^{B2} is hydrogen, optionally substituted C₁₋₆ alkyl, optionally substituted C₁₋₆ acyl, or a nitrogen protecting group. In certain embodiments, R^{B2} is hydrogen. In certain embodiments, R^{B2} is optionally substituted C₁₋₆ alkyl.

In certain embodiments, R^{B3} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, or optionally substituted 5-10 membered heteroaryl.

In certain embodiments, R^{B3} is optionally substituted C₁₋₆ alkyl. In certain embodiments, R^{B3} is unsubstituted C₁₋₆ alkyl. In certain embodiments, R^{3B} is tert-butyl.

In certain embodiments, R^{B4} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group. In certain embodiments, R^{B4} is hydrogen. In certain embodiments, R^{B4} is optionally substituted C₁₋₆ alkyl.

In certain embodiments, R^{B5} is hydrogen, optionally substituted C₁₋₆ alkyl, optionally substituted C₁₋₆ acyl, or a nitrogen protecting group. In certain embodiments, R^{B5} is hydrogen. In certain embodiments, R^{B5} is optionally substituted C₁₋₆ alkyl.

In certain embodiments, each instance of R^{B6} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R^{B6} is hydrogen. In certain embodiments, at least one instance of R^{B6} is optionally substituted C₁₋₆ alkyl. In certain embodiments, at least one instance of R^{B6} is halogen.

In certain embodiments, R^{B7} is hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, R^{B7} is hydrogen. In certain embodiments, R^{B7} is halogen.

In certain embodiments, R^{B7} is optionally substituted C₁₋₆ alkyl. In certain embodiments, R^{B7} is unsubstituted C₁₋₆ alkyl. In certain embodiments, R^{B7} is unsubstituted C₁₋₃ alkyl. In certain embodiments, R^{B7} is methyl.

In certain embodiments, R^{B8} is hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, R^{B8} is hydrogen. In certain embodiments, R^{B8} is optionally substituted C₁₋₆ alkyl. In certain embodiments, R^{B8} is halogen.

In certain embodiments, R^{B9} is hydrogen or optionally substituted C₁₋₆ alkyl. In certain embodiments, R^{B9} is hydrogen. In certain embodiments, R^{B9} is optionally substituted C₁₋₆ alkyl. In certain embodiments, R^{B9} is unsubstituted C₁₋₆ alkyl. In certain embodiments, R^{B9} is unsubstituted C₁₋₃ alkyl. In certain embodiments, R^{B9} is methyl.

In certain embodiments, R^{B10} is hydrogen or optionally substituted C₁₋₆ alkyl. In certain embodiments, R^{B10} is hydrogen. In certain embodiments, R^{B10} is optionally substituted C₁₋₆ alkyl.

In certain embodiments, R^{B9} and R^{B10} are hydrogen. In certain embodiments, R^{B9} is optionally substituted C₁₋₆ alkyl; and R^{B10} is hydrogen. In certain embodiments, R^{B9} is unsubstituted C₁₋₆ alkyl; and R^{B10} is hydrogen. In certain embodiments, R^{B9} is unsubstituted C₁₋₃ alkyl; and R^{B10} is hydrogen. In certain embodiments, R^{B9} is methyl; and R^{B10} is hydrogen.

In certain embodiments, R^{B9} and R^{B10} are joined together with the intervening atoms to form optionally substituted C₃₋₈ carbocyclyl or optionally substituted 3-8 membered heterocyclyl.

In certain embodiments, each instance of R^{B11} is independently halogen or optionally substituted C₁-C₆ alkyl. In certain embodiments, R^{B11} is optionally substituted C₁₋₆ alkyl. In certain embodiments, R^{B11} is halogen.

In certain embodiments, q is 0, 1, 2, 3, 4, or 5. In certain embodiments, q is 0.

In certain embodiments, v2 is 0, 1, 2, 3, or 4. In certain embodiments, v2 is 0.

In certain embodiments, each instance of R^{Ca} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, at least one instance of R^{Ca} is hydrogen. In certain embodiments, at least one instance of R^{Ca} is optionally substituted C₁₋₆ alkyl. In certain embodiments, at least one instance of R^{Ca} is halogen.

In certain embodiments, R^{Cb} is hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S}. In certain embodiments, R^{Cb} is hydrogen. In certain embodiments, at least one instance of R^{Cb} is optionally substituted C₁₋₆ alkyl. In certain embodiments, at least one instance of R^{Cb} is halogen.

In certain embodiments, each instance of R^{Cc} is independently halogen or optionally substituted C₁-C₆ alkyl. In certain embodiments, at least one instance of R^{Cc} is optionally substituted C₁₋₆ alkyl. In certain embodiments, at least one instance of R^{Cc} is halogen.

In certain embodiments, R^{Cd} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, or optionally substituted C₁₋₆ acyl.

In certain embodiments, R^{Cd} is optionally substituted C₃₋₈ carbocyclyl. In certain embodiments, R^{Cd} is optionally substituted C₅₋₇ carbocyclyl. In certain embodiments, R^{Cd} is optionally substituted C₆ carbocyclyl. In certain embodiments, R^{Cd} is unsubstituted C₃₋₈ carbocyclyl. In certain embodiments, R^{Cd} is unsubstituted C₅₋₇ carbocyclyl. In certain embodiments, R^{Cd} is:

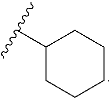 [SMILES: *C1CCCCC1]

In certain embodiments, R^{Ce} is hydrogen, optionally substituted C₁₋₆ alkyl, optionally substituted C₁₋₆ acyl, or a nitrogen protecting group. In certain embodiments, R^{Ce} is hydrogen. In certain embodiments, R^{Ce} is optionally substituted C₁₋₆ alkyl.

In certain embodiments, R^{Cf} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, or optionally substituted C₁₋₆ acyl. In certain embodiments, R^{Cf} is hydrogen.

In certain embodiments, R^{Cf} is optionally substituted C₁₋₆ alkyl. In certain embodiments, R^{Cf} is unsubstituted C₁₋₆ alkyl. In certain embodiments, R^{Cf} is unsubstituted C₁₋₃ alkyl. In certain embodiments, R^{Cf} is methyl.

In certain embodiments, R^{Cg} is hydrogen, optionally substituted C₁₋₆ alkyl, optionally substituted C₁₋₆ acyl, or a nitrogen protecting group. In certain embodiments, R^{Cg} is hydrogen.

In certain embodiments, R^{Cg} is optionally substituted C₁₋₆ alkyl. In certain embodiments, R^{Cg} is unsubstituted C₁₋₆ alkyl. In certain embodiments, R^{Cg} is unsubstituted C₁₋₃ alkyl. In certain embodiments, R^{Cg} is methyl.

In certain embodiments, R^{Ch} is hydrogen, optionally substituted C₁₋₆ alkyl, optionally substituted C₁₋₆ acyl, or a nitrogen protecting group. In certain embodiments, R^{Ch} is hydrogen. In certain embodiments, R^{Ch} is optionally substituted C₁₋₆ alkyl.

In certain embodiments, R^{Cg} is optionally substituted C₁₋₆ alkyl; and R^{Ch} is hydrogen. In certain embodiments, R^{Cg} is unsubstituted C₁₋₆ alkyl; and R^{Ch} is hydrogen. In certain embodiments, R^{Cg} is unsubstituted C₁₋₃ alkyl; and R^{Ch} is hydrogen. In certain embodiments, R^{Cg} is methyl; and R^{Ch} is hydrogen.

In certain embodiments, R^{Cg} and R^{Ch} are joined together with the intervening atoms to form optionally substituted optionally substituted 3-8 membered heterocyclyl or optionally substituted 5-10 membered heteroaryl;

In certain embodiments, y is 0, 1, 2, 3, or 4. In certain embodiments, y is 0.

In certain embodiments, z is 0, 1, 2, 3, 4, 5, 6, or 7. In certain embodiments, z is 0.

In certain embodiments, each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group.

In certain embodiments, each instance of R^{O} is independently hydrogen, optionally substituted C₁₋₆ alkyl, optionally substituted C₁₋₆ acyl, or an oxygen protecting group. In certain embodiments, at least one instance of R^{O} is hydrogen. In certain embodiments, at least one instance of R^{O} is optionally substituted C₁₋₆ alkyl.

In certain embodiments, each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group.

In certain embodiments, two R^{N} bonded to the same nitrogen atom are taken together with the intervening atoms to form optionally substituted optionally substituted 3-8 membered heterocyclyl or optionally substituted 5-10 membered heteroaryl.

In certain embodiments, each instance of R^{N} is independently hydrogen, optionally substituted C₁₋₆ alkyl, optionally substituted C₁₋₆ acyl, or a nitrogen protecting group. In certain embodiments, at least one instance of R^{N} is hydrogen. In certain embodiments, at least one instance of R^{N} is optionally substituted C₁₋₆ alkyl.
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group.

In certain embodiments, each instance of R^{S} is independently hydrogen, optionally substituted C₁₋₆ alkyl, optionally substituted C₁₋₆ acyl, or a sulfur protecting group. In certain embodiments, at least one instance of R^{S} is hydrogen. In certain embodiments, at least one instance of R^{S} is optionally substituted C₁₋₆ alkyl.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Pharmaceutical Compositions, Kits, and Administration

The present disclosure provides pharmaceutical compositions comprising a compound provided herein (e.g., a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof). The pharmaceutical composition may comprise one or more pharmaceutically acceptable carriers/excipients. In certain embodiments, a compound described herein is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives.

In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions can be conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form. Low-boiling propellants generally include liquid propellants having a boiling point of below 65° F. at atmospheric pressure. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

A pharmaceutical composition described herein can be prepared, packaged, and/or sold in a formulation for buccal administration. Such formulations may, for example, be in the form of tablets and/or lozenges made using conventional methods. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically, contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The additional pharmaceutical agents include, but are not limited to, anti-proliferative agents, anti-cancer agents, anti-angiogenesis agents, steroidal or non-steroidal anti-inflammatory agents (NSAIDs), immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, and hormones.

In certain embodiments, the additional pharmaceutical agent is an anti-proliferative agent. In certain embodiments, the additional pharmaceutical agent is an anti-cancer agent. “Anti-cancer agents” encompass biotherapeutic anti-cancer agents as well as chemotherapeutic agents.

In certain embodiments, the additional pharmaceutical agent is a protein kinase inhibitor. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs, hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, and other agents that promote differentiation.

Exemplary biotherapeutic anti-cancer agents include, but are not limited to, interferons, cytokines, vaccines, hematopoietic growth factors, monoclonal serotherapy, immunostimulants and/or immunodulatory agents, immune cell growth factors, and antibodies.

In certain embodiments, a compound or composition is used in combination with an immunotherapy. In certain embodiments, a compound or composition is used in combination with an immune checkpoint inhibitor. Checkpoint inhibitors can be broken down into at least 4 major categories: i) agents such as antibodies that block an inhibitory pathway directly on T cells or natural killer (NK) cells (e.g., PD-1 targeting antibodies, antibodies targeting TIM-3, and antibodies targeting LAG-3, 2B4, CD160, A2aR, BTLA, CGEN-15049, or KIR); ii) agents such as antibodies that activate stimulatory pathways directly on T cells or NK cells (e.g., antibodies targeting OX40, GITR, or 4-1BB); iii) agents such as antibodies that block a suppressive pathway on immune cells or rely on antibody-dependent cellular cytotoxicity to deplete suppressive populations of immune cells (e.g., CTLA-4 targeting antibodies, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., antibodies targeting PD-L1, and antibodies targeting B7-H3, B7-H4, Gal-9, or MUC1).

In certain embodiments, the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compounds described herein or pharmaceutical compositions can be administered in combination with an anti-cancer therapy including, but not limited to, surgery, radiation therapy, and transplantation (e.g., stem cell transplantation, bone marrow transplantation).

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form a single unit dosage form. Thus, in one aspect, provided are kits including a first container comprising a compound or pharmaceutical composition described herein. In certain embodiments, the kits are useful for treating a disease (e.g., cancer) in a subject in need thereof. In certain embodiments, the kits are useful for preventing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease in a subject in need thereof. A kit described herein may include one or more additional pharmaceutical agents described herein as a separate composition.

### Methods of Treatment and Uses

Compounds provided herein are degraders of GRK2 proteins and are therefore useful in, e.g., treating and/or preventing diseases (e.g., proliferative diseases (e.g., cancer), cardiovascular diseases) in a subject, inhibiting tumor growth in a subject, degrading GRK2 proteins in vitro or in vivo, etc. Compounds provided herein can also degrade of GRK3 proteins and are therefore useful in, e.g., treating and/or preventing diseases (e.g., proliferative diseases (e.g., cancer), cardiovascular diseases) in a subject, inhibiting tumor growth in a subject, degrading GRK3 proteins in vitro or in vivo, etc.

Provided herein are methods of treating and/or preventing a disease in a subject comprising administering to the subject a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in treating and/or preventing a disease in a subject. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments.

In certain embodiments, the disease is a GRK2-related disease. In certain embodiments, the disease is a GRK3-related disease.

In certain embodiments, the disease is a hematological disease, an infection, a cardiovascular disease, (e.g., cardiac failure, cardiac hypertrophy, hypertension), a proliferative disease (e.g., cancer), an endocrinological disease, a metabolic disease, a gastroenterological disease, a respiratory disease, inflammation (e.g., inflammatory bowel disease), a neurological disease, opioid addiction, or an urological disease.

In certain embodiments, the disease is a proliferative disease (e.g., cancer). Provided herein are methods of treating a proliferative disease (e.g., cancer) in a subject comprising administering to the subject a therapeutically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in treating a proliferative disease (e.g., cancer) in a subject. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for treating proliferative diseases (e.g., cancer). In certain embodiments, the proliferative disease is cancer. In certain embodiments, the cancer is pancreatic cancer.

A “proliferative disease” refers to a disease that occurs due to abnormal growth or extension by the multiplication of cells (See, e.g., Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located.

In certain embodiments, the proliferative disease to be treated is cancer. In certain embodiments, the cancer is a GRK2-related cancer. In certain embodiments, the cancer is a GRK3-related cancer.

The term “cancer” refers to a class of diseases characterized by the development of abnormal cells that proliferate uncontrollably and have the ability to infiltrate and destroy normal body tissues. In certain embodiments, the cancer is a solid tumor. In certain embodiments, the cancer is a hematopoietic cancer (i.e., hematological cancer).

In certain embodiments, the cancer is a hematopoietic cancer (e.g., leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomads, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; immunocytic amyloidosis). In certain embodiments, the cancer is leukemia. In certain embodiments, the cancer is acute lymphoblastic leukemia (ALL). In certain embodiments, the cancer is early T-cell precursor (ETP)-acute lymphoblastic leukemia (ALL).

In certain embodiments, the cancer is liver cancer (e.g., hepatocellular cancer (HCC) (e.g., hepatocellular carcinoma, hepatoblastoma, hepatocellular adenoma), malignant hepatoma, hemangiomas, biliary cancer (e.g., cholangiocarcinoma)).

In certain embodiments, the cancer is musculoskeletal cancer (e.g., bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer, synovioma).

In certain embodiments, the cancer is a nervous system cancer (e.g., brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma)). In certain embodiments, the disease to be treated is a brain tumor. In certain embodiments, the disease is pleomorphic xenoanthrocytoma (PXA). In certain embodiments, the disease is pediatric pleomorphic xenoanthrocytoma (PXA).

In certain embodiments, the cancer is selected from endocrine/exocrine cancers (e.g., thyroid cancer (e.g., papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic andenocarcinoma, intraductal papillary mucinous neoplasm (IPMN), Islet cell tumors, ductal adenocarcinoma, insulinoma, glucagonoma, vipoma), adrenal gland cancer, neuroendocrine cancer (e.g., gastroenteropancreatic neuroendoctrine tumor (GEP-NET), carcinoid tumor), sebaceous gland carcinoma, sweat gland carcinoma). In certain embodiments, the cancer is sweat gland cancer (e.g., sweat gland carcinoma).

In certain embodiments, the cancer is head and neck cancer (e.g., squamous cell carcinoma of the head and neck (SCCHN), adenoid cystic carcinoma).

In certain embodiments, the cancer is oral cancer (e.g., buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer), salivary gland cancer).

In certain embodiments, the cancer is esophageal cancer (e.g., esophageal squamous cell carcinoma, esophageal adenocarcinoma, Barrett's adenocarcinoma, esophageal leiomyosarcoma).

In certain embodiments, the cancer is gastrointestinal cancer (e.g., anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer, large intestine cancer).

In certain embodiments, the cancer is cardiovascular cancer (e.g., primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma, cardiac rhabdomyoma).

In certain embodiments, the cancer is lung cancer (e.g., bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma, papillary adenocarcinoma).

In certain embodiments, the cancer is a genitourinary cancer (e.g., bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma), penile cancer (e.g., Paget's disease of the penis and scrotum)).

In certain embodiments, the cancer is a gynecological cancer (e.g., breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer), endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer, vulvar cancer (e.g., Paget's disease of the vulva) vaginal cancer, fallopian tube cancer).

In certain embodiments, the cancer is skin cancer (e.g., squamous cell carcinoma (SCC), keratoacanthoma (KA), melanoma, basal cell carcinoma (BCC), dermatofribroma).

In certain embodiments, the cancer is a soft tissue cancer (e.g., intraepithelial neoplasms, epithelial carcinomas, epithelial sarcomas, adenocarcinomas, adenomas, fibrosarcomas, fibromas, liposarcomas, lipomas, myxomas, teratomas).

In certain embodiments, the cancer is skin cancer (e.g., melanoma), breast cancer, ovarian cancer, prostate cancer, gliomas, thyroid cancer, pancreatic cancer, bile duct cancer, urinary tract cancer, head and neck cancer, gastric cancer, rhabdoid cancer, mesothelioma, cervical cancer, liver cancer, colorectal cancer, lymphoma, lung cancer, leukemia, or kidney cancer. In certain embodiments, the cancer is pancreatic cancer.

Additionally, provided herein are methods of inhibiting tumor growth in a subject comprising administering to the subject an effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting tumor growth in a subject. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for inhibiting tumor growth.

In certain embodiments, the tumor is a GRK2-related tumor. In certain embodiments, the tumor is a GRK3-related tumor.

In certain embodiments, treating cancer and/or inhibiting tumor growth can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

In certain embodiments, treating cancer and/or inhibiting tumor growth may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

In certain embodiments, treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

In certain embodiments, treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the present disclosure. An increase in average survival time of a population may also be measured, for example, by calculating for a population the average length of survival following completion of a first round of treatment with the compound of the present disclosure.

In certain embodiments, treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the present disclosure. A decrease in the mortality rate of a population may also be measured, for example, by calculating for a population the average number of disease-related deaths per unit time following completion of a first round of treatment with the compound of the present disclosure.

In certain embodiments, treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the present disclosure. An increase in average progression-free survival time of a population may also be measured, for example, by calculating for a population the average length of progression-free survival following completion of a first round of treatment with the compound of the present disclosure. “Progression-free survival” as used herein refers to the length of time during and after medication or treatment during which the disease being treated (e.g., cancer) does not get worse.

Also provided herein are methods of treating and/or preventing a cardiovascular disease in a subject comprising administering to the subject a therapeutically and/or prophylactically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in treating and/or preventing a cardiovascular disease in a subject. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for treating a cardiovascular disease.

In certain embodiments, the cardiovascular disease is a GRK2-related cardiovascular disease. In certain embodiments, the cardiovascular disease is a GRK2-related cardiovascular disease.

In certain embodiments, the disease is a cardiovascular disease. A “cardiovascular disease” is a disease involving the heart and/or blood vessels. In certain embodiments, the disease is atherogenesis or atherosclerosis. In certain embodiments, the disease is arterial stent occlusion, heart failure (e.g., congestive heart failure), a coronary arterial disease, myocarditis, pericarditis, a cardiac valvular disease, stenosis, restenosis, in-stent-stenosis, angina pectoris, myocardial infarction, acute coronary syndromes, coronary artery bypass grafting, a cardio-pulmonary bypass procedure, endotoxemia, ischemia-reperfusion injury, cerebrovascular ischemia (stroke), renal reperfusion injury, embolism (e.g., pulmonary, renal, hepatic, gastro-intestinal, or peripheral limb embolism), or myocardial ischemia.

Also provided herein are methods of treating opioid addiction in a subject comprising administering to the subject a therapeutically effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in treating opioid addiction in a subject. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for treating opioid addiction.

Also provided herein are methods for degrading a GRK2 protein in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in degrading a GRK2 protein in vivo or in vitro. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for degrading a GRK2 protein in vivo. In certain embodiments, the degrading occurs in vivo. In certain embodiments, the degrading occurs in vitro. In certain embodiments, the degradation is selective for GRK2, i.e., selective for GRK2 over other proteins (e.g., over other protein kinases, e.g., over other GRK family member proteins). In certain embodiments, the method or use further comprises determining the level of a protein (e.g., GRK2) in the subject or in vitro.

Also provided herein are methods for inhibiting the activity of GRK2 in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting the activity of GRK2 in vivo or in vitro. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for inhibiting the activity of GRK2 in vivo. In certain embodiments, the inhibiting occurs in vivo. In certain embodiments, the inhibiting occurs in vitro. In certain embodiments, the inhibition is selective for GRK2, i.e., selective for GRK2 over other proteins (e.g., over other protein kinases, e.g., over other GRK family member proteins). In certain embodiments, the method or use further comprises determining the level of a protein (e.g., GRK2) in the subject or in vitro.

Also provided herein are methods for degrading a GRK3 protein in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in degrading a GRK3 protein in vivo or in vitro. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for degrading a GRK3 protein in vivo. In certain embodiments, the degrading occurs in vivo. In certain embodiments, the degrading occurs in vitro. In certain embodiments, the degradation is selective for GRK3, i.e., selective for GRK3 over other proteins (e.g., over other protein kinases, e.g., over other GRK family member proteins). In certain embodiments, the method or use further comprises determining the level of a protein (e.g., GRK3) in the subject or in vitro.

Also provided herein are methods for inhibiting the activity of GRK3 in vivo or in vitro with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting the activity of GRK3 in vivo or in vitro. Also provided herein are uses of compounds of Formulae (A-I), (B-I), and (C-I), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of medicaments for inhibiting the activity of GRK3 in vivo. In certain embodiments, the inhibiting occurs in vivo. In certain embodiments, the inhibiting occurs in vitro. In certain embodiments, the inhibition is selective for GRK3, i.e., selective for GRK2 over other proteins (e.g., over other protein kinases, e.g., over other GRK family member proteins). In certain embodiments, the method or use further comprises determining the level of a protein (e.g., GRK2) in the subject or in vitro.

It would be understood that in vivo methods provided herein comprise administering to a subject an effective amount of a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof.

In certain embodiments, in vitro methods provided herein can be carried out, for example, in a cell line, assay, biological sample, etc. In certain embodiments, methods for degrading a GRK2 protein and/or inhibiting the activity of GRK2 in vitro comprise contacting a GRK2 protein with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for degrading a GRK2 protein and/or inhibiting the activity of GRK2 in a cell comprise contacting the cell with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for degrading GRK2 protein and/or inhibiting the activity of GRK2 in a biological sample comprise contacting the biological sample with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof.

In certain embodiments, methods for degrading a GRK3 protein and/or inhibiting the activity of GRK3 in vitro comprise contacting a GRK3 protein with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for degrading a GRK3 protein and/or inhibiting the activity of GRK2 in a cell comprise contacting the cell with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for degrading GRK3 protein and/or inhibiting the activity of GRK2 in a biological sample comprise contacting the biological sample with a compound of Formula (A-I), (B-I), or (C-I), or a pharmaceutically acceptable salt, stereoisomer, tautomer, isotopically labeled derivative, solvate, hydrate, polymorph, co-crystal, or prodrug thereof, or a pharmaceutical composition thereof.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### GRK2 Enzyme Inhibition

### Example 1. GRK2 Inhibition Assay

Assay: GRK2 enzyme (1 nM final concentration) was diluted in 25 mM HEPES, 10 mM MgCl₂, 2 mM DTT, 0.01% Tween-20, and 1 mM EGTA. Then the GRK2 mixture was added into ProxiPlate-384 white plate and pre-incubated for 30 min with test compounds at room temperature. ATP (7 μM final concentration) and Ulight TopoIIα (50 nM final concentration) were added into the assay plate to initiate the reaction and the mixture was incubated at room temperature for 90 min. Then, Eu anti-TopoIIα (0.12 nM final concentration), BSA (0.01% final concentration), and EDTA (11 mM final concentration) in LANCE assay buffer were added into each well. After 60 min incubation time at room temperature, TR-FRET signal was measured by EnVision plate reader.

Results: As shown in Table 1 and Table 4 below, compounds of the present disclosure were found to inhibit GRK2.

### Example 2. ADPGlo GRK2 Inhibition Assay

Assay: GRK2 (7.5 nM) was incubated with ATP (10 μM) and GRKtide (0.3 mg/mL) in 5 μL of assay buffer (see above) for 180 min at room temperature. HTS was performed using 1 μM compound. Compounds were dissolved in 100% DMSO, serially diluted tree-fold from 100 μM concentration to 46 nM and transferred (50 nL) into assay ready plate.

Materials: GRK2 was purchased from SignalChem (Cat #A14-10G, Lot #X645-3). Substrate GRKtide was from SignalChem (Cat #G46-58, Lot #R339-6). ADP-Glo Kinase Assay from Promega (Cat #V9102). Assay buffer consisted of 25 mM HEPES (pH7.5), 10 mM MgCl₂, 0.01% Tween-20, 1 mM DDT. 384-well white plates were from Greiner Bio-Rad (Item #784075).

HTS protocol: Take 384 well plate with 50 nL of compound in columns 3-22/DMSO solution 1-2,23-24. Add 2.5 μL assay buffer to columns 23 and 24 using Thermo Scientific Multidrop Combi Dispenser. Add 2.5 μL of 2× enzyme solution (15 nM in 1× assay buffer) using Thermo Scientific Multidrop Combi Reagent Dispenser to all columns except of 23 and 24. Incubate for 15 minutes. Fill plate with 2.5 μL of 2× substrate mix (20 μM ATP and 0.6 mg/mL GRKtide in 1× assay buffer) using Thermo Scientific Multidrop Combi Reagent Dispenser. Spin 15 seconds at 1000 rpm and incubate for 180 min at room temperature. Add 5 μL ADP-Glo reagent to all the wells. Spin 15 seconds at 1000 rpm and incubate for 40 min at room temperature. Add 10 μL Detection solution to all the wells. Incubate for 30 min at room temperature. Read plate in Luminescent mode on BMG PheraStar FSX (gain=3600).

Data Analysis: Data were analyzed in GraphPad Prism 8.0.2. Each HTS plate contains compounds in columns 3-22, controls (enzyme, no compound) in columns 1 and 2, and blanks (no enzyme) in columns 23 and 24. HTS percent inhibition was calculated for each compound from the signal in luminescence units and the mean of the plate controls and the mean of the plate blanks using the following equation: % Inhibition=100*(1−((signal−blank mean)/(control mean−blank mean))). At the final stage of data processing, we obtain dose-response curves, tables and SDF-files with the results of screening for each substance.

Results: As shown in Table 1 below, compounds of the present disclosure were found to inhibit GRK2.

### Example 3. Second ADPGlo GRK2 Inhibition Assay

Assay: GRK2 (2 nM) was incubated with ATP (20 μM) and GRKtide (1.2 mg/mL) in 11 μL of assay buffer (see above) for 180 min at room temperature. Compounds were dissolved in 100% DMSO, serially diluted 3.16-fold from 100 μM with 10 concentration points and transferred as 100× into assay plate (final compounds concentration in the reaction plate from 1 μM).

Materials: GRK2 was purchased from SignalChem (Cat #A14-10G, Lot #X645-3). Substrate GRKtide was from SignalChem (Cat #G46-58, Lot #R339-6). ADP-Glo Kinase Assay from Promega (Cat #V9102). Assay buffer consisted of 20 mM HEPES (pH7.5), 20 mM MgCl2, 0.01% Tween-20, 0.05 mM DDT. 384-well white plates were from Corning (Cat #4513).

HTS protocol: Add 5.5 μL of 2× substrate-enzyme mix into all columns except of 1-2 and 24 of reaction plate. Spin 15 seconds at 200 g. Transfer 1 ul of compounds from the plate with serial dilutions (384 well plate with 22 uL of compound in columns 3-22/DMSO solution 1-2,23-24) to Dilution plate (384 well plate with 49 uL of buffer with 2×40 μM ATP). After mixing transfer 5.5 ul from Dilution plate to Reaction plate (to 5.5 μL of 2× substrate-enzyme mix). Spin 15 seconds at 200 g and incubate for 180 min at room temperature with sealing. Add 3 μL ADP-Glo reagent to all the wells. Spin 15 seconds at 200 g and incubate for 40 min at room temperature. Add 6 μL Detection solution to all the wells. Incubate for 30 min at room temperature. Read plate using ClarioStar Plus (BMG).

Data Analysis: Data were analyzed in GraphPad Prism 5.0.2. Each HTS plate contains compounds in columns 3-22, controls (enzyme, no compound) in column 23, and blanks (no enzyme) in columns 2. HTS percent inhibition was calculated for each compound from the signal in luminescence units and the mean of the plate controls and the mean of the plate blanks using the following equation: % Inhibition=100*(1-(signal-blank mean)/(control mean-blank mean)).

Results: As shown in Table 1 below, compounds of the present disclosure were found to inhibit GRK2.

TABLE 1
GRK2 Enzyme Inhibition Data

| | | ADP Glo | Second ADPGlo |
|---|---|---|---|
| | Enzymatic GRK2 | Enzymatic GRK2 | Enzymatic GRK2 |
| | Assay: IC₅₀ (nM) | Assay: IC₅₀ (nM) | Assay: IC₅₀ (nM) |
| Compound | (Example 1)* | (Example 2)* | (Example 3)* |
| D52 | | | A |
| D74 | B | | |
| D53 | B | | |
| D75 | A | | |
| D54 | A | | |
| D76 | | | A |
| D55 | A | | |
| D56 | | | A |
| D77 | | | A |
| D57 | A | | |
| D78 | A | | |
| D79 | A | | |
| D80 | A | | |
| D59 | A | | |
| D60 | | | A |
| D82 | | | A |
| D83 | | | A |
| D62 | | | A |
| D84 | A | | |
| D63 | A | | |
| D64 | A | | |
| D85 | | | A |
| D65 | | | A |
| D86 | A | | |
| D66 | A | | |
| D67 | A | | |
| D87 | A | | |
| D88 | B | | |
| D102 | A | | |
| D89 | A | | |
| D90 | A | | |
| D91 | A | | |
| D92 | A | | |
| D103 | A | | |
| D93 | | | A |
| D68 | | | C |
| D94 | | | B |
| D95 | | | A |
| D69 | | | A |
| D70 | | | A |
| D71 | | A | |
| D72 | | A | |
| D73 | | | A |
| D96 | | | A |
| D97 | | | A |
| D98 | | | A |
| D99 | | | A |
| D100 | | A | A |
| D101 | | A | A |

*Scale: A < 100 nM; 100 nM < B < 1 uM; C > 1 uM

### Proliferation Data

### Example 4: Cellular Proliferation Assay

In order to identify compounds specific for GRK2, we generated two isogenic cell line pairs of the pancreatic cancer cell line PAXF1657, a pair of cells stably overexpressing GRK2 cDNA or the control empty vector and a cell line pair of PAXF1657 GRK2 knockout clonal cells versus non-targeting control clonal cells. The goal was to identify compounds that effectively impair proliferation in PAXF1657 empty vector or non-targeting control cells, but not in GRK2 cDNA overexpressing or GRK2 knockout cells, thereby identifying compounds superior to compounds of Okawa et al. J. Med. Chem. 2017, 60, 6942-6990.

Proliferation Assay: Cellular anti-proliferative activity of compounds was assessed by using the pancreatic cancer cell line, PAXF1657, stably overexpressing GRK2 cDNA or the control empty vector, as well as the parental urinary bladder cancer cell line, 5637. In addition, we also took advantage of GRK2 knockout cell lines that were generated via CRISPR. Cell lines were seeded into tissue culture treated, white-walled, 96-well plates at a density of 500 cells/well in RPMI1640 media supplemented with 10% H.I. FBS and penicillin/streptomycin. Plates were incubated overnight at 37° C., 5% CO₂ to allow cells to adhere to the wells. GRK2 inhibitors were added to the cells using a 10-point dilution series with a final concentration ranging from 30 μM-0.0002 μM in 0.3% DMSO. At the time of compound addition, a set of plates, that were not treated with compounds, were collected and cell viability was measured using CellTiter-Glo (Promega). CellTiter-Glo reagent was added to the designed plates and luminescence was measuring using a Biotek Synergy plate reader. The compound treated cells were incubated for 3 days at 37° C., 5% CO₂. The media was then aspirated from each well and replaced with fresh media containing GRK2 inhibitors. The compound treated cells were then incubated for an additional 4 days at 37° C., 5% CO₂. Cell viability was assessed and at end of the 7-day compound treatment by CellTiterGlo.

Results: As shown in Table 2 and Table 4, compounds provided herein were found to inhibit proliferation the PAXF1657 control cell line significantly more than a PAXF1657 cell line that overexpresses GRK2. This indicates the inhibition of proliferation by the compounds provided herein is the result of GRK2 inhibition by the compounds.

TABLE 2
Cellular Proliferation Data

| | | 7-day |
|---|---|---|
| | 7-day | proliferation assay: |
| | proliferation assay: | IC₅₀ GeoMean (uM) |
| | IC₅₀ GeoMean (uM) | [Cell Line: PAXF1657 |
| Compound | [Cell Line: PAXF1657 EV]* | KO Clone #4]* |
| D56 | B | C |
| D77 | A | C |
| D57 | C | C |
| D78 | A | B |
| D79 | A | C |
| D58 | B | C |
| D80 | C | C |
| D59 | C | C |
| D81 | C | C |
| D60 | C | C |
| D82 | A | C |
| D61 | C | C |
| D83 | B | C |
| D62 | C | C |
| D63 | C | C |
| D64 | C | C |
| D85 | B | C |
| D65 | A | C |
| D86 | C | C |
| D66 | C | C |
| D107 | C | C |
| D67 | B | C |
| D87 | B | C |
| D102 | A | B |
| D90 | B | C |
| D113 | C | C |
| D114 | C | C |
| D115 | C | B |
| D91 | A | B |
| D92 | A | C |
| D103 | B | B |
| D72 | B | B |
| D73 | B | C |
| D96 | B | C |
| D97 | C | B |
| D99 | A | B |
| D100 | C | C |
| D101 | A | B |

*Scale: A < 1 uM; 1 uM < B < 10 uM; C > 10 uM

### GRK2 Degradation Data

### Example 5: GRK2 Degradation Assay

General Protocol: Degradation Assay and Western Blot. PAXF1657 cells were plated in 6-well, 24-well plates or 60 mm tissue culture treated dishes and incubated at 37° C. until 70% confluency. The media was aspirated, and the cells were treated with fresh media containing the compounds in the concentration range of 0 μM to 10 μM for a period of 6 to 24 hours. At the desired time points the cells were washed twice with cold PBS and lysed with lysis buffer (ice cold PBS+1% NP40 with 1/100 protease/phosphatase inhibitors) on ice for 8 mins followed by sonication at 70% intensity pulses for 10 seconds. The cells were centrifuged at 20000 g for 8 min at 4° C. The supernatants were collected and stored at −80° C.

Samples were mixed with NuPAGE LDS sample buffer and NuPAGE sample reducing buffer (3×), vortexed and heated at 95° C. for 10 min, and then analyzed by SDS-PAGE (NuPAGE 4-12%) followed by transfer to nitrocellulose membrane. Blocking was achieved with 5% milk in TBST (TBS with 1% tween 20) for 1 hour. The membranes were washed three times with TBST and incubated with GRK2 or R-actin antibodies in 5% milk in TBST at 4° C. overnight. The membranes were washed three times with TBST and incubated with anti-mouse IgG-HRP or anti-rabbit IgG-HRP at room temperature for 2 hours in 5% milk in TBST. The membranes were washed three times with TBST and developed with ECL Dura, ECL femto or ECL atto substrates.

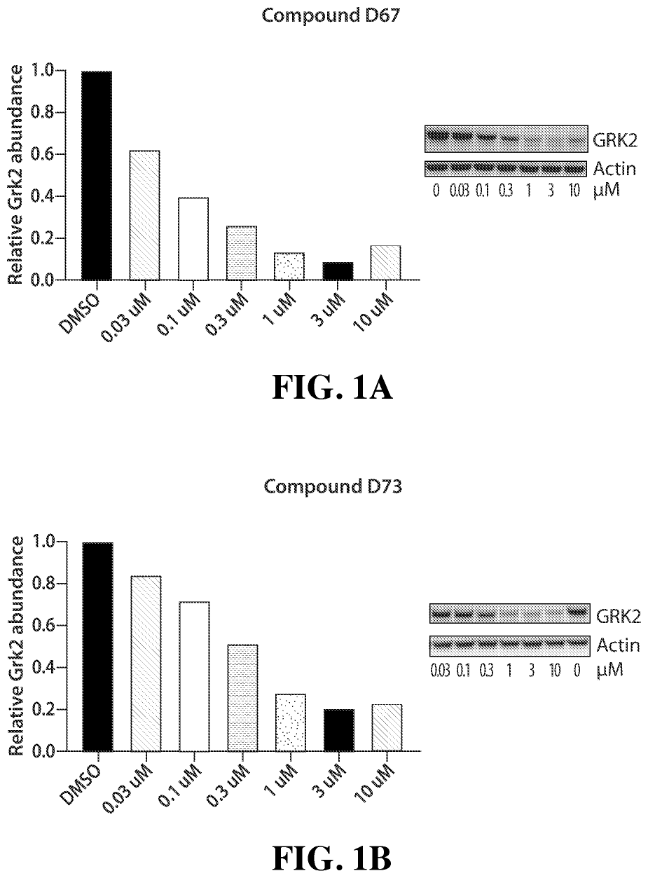

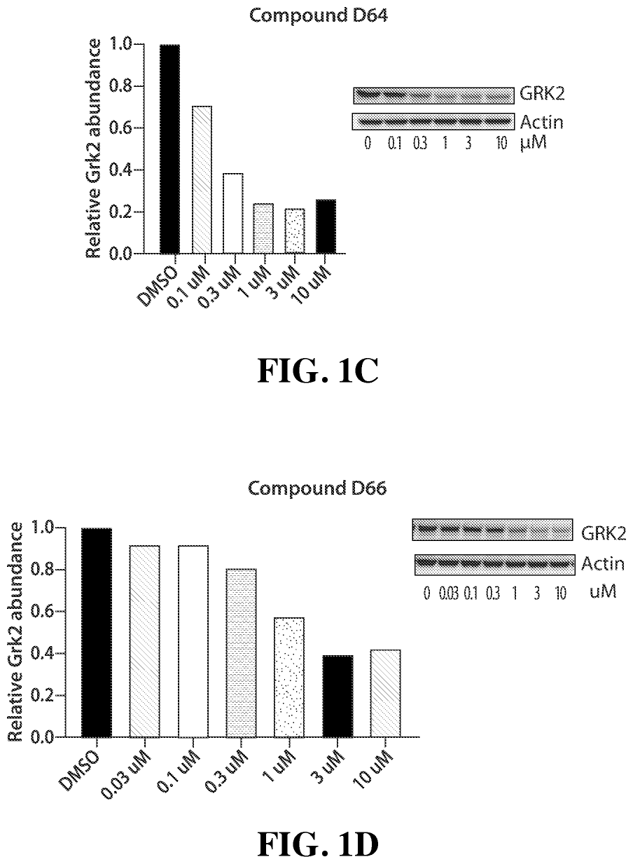

Results: As shown below in Table 3, compounds provided herein are degraders of GRK2 (represented as % GRK2 degradation). As shown in FIGS. 1A-1D. Dose dependent Grk2 degradation of D67 (A), D73 (B), D64 (C), and D66 (D) after 24-hour treatment in PAXF1657 cells.

TABLE 3
Degradation of GRK2

| | % GRK2 | % GRK2 | % GRK2 | % GRK2 | % GRK2 | % GRK2 |
|---|---|---|---|---|---|---|
| | degradation | degradation | degradation | degradation | degradation | degradation |
| | at 0.03 uM in | at 0.1 uM in | at 0.3 uM in | at 1 uM in | at 3 uM in | at 10 uM in |
| | PAXF1657 | PAXF1657 | PAXF1657 | PAXF1657 | PAXF1657 | PAXF1657 |
| Compound | EV (24 hrs) | EV (24 hrs) | EV (24 hrs) | EV (24 hrs) | EV (24 hrs) | EV (24 hrs) |
| D101 | | | | 18% | | 10% |
| D100 | | | | 22% | | 39% |
| D99 | | | | 44% | 13% | |
| D97 | | | | 11% | | |
| D96 | | | 19% | 31% | 6% | |
| D73 | 15% | 28% | 48% | 67% | 76% | 76% |
| D72 | | | 36% | 59% | 64% | |
| D87 | | | | 23% | 18% | |
| D92 | | | 34% | 12% | 5% | |
| D91 | 37% | 28% | 42% | 51% | 16% | −4% |
| D79 | | | 52% | 54% | 8% | |
| D78 | | | −12% | −21% | −18% | |
| D65 | | | 36% | 64% | 67% | |
| D67 | 38% | 60% | 74% | 87% | 90% | 83% |
| D57 | | | 70% | 82% | 85% | |
| D107 | | | | 19% | 20% | |
| D115 | | | | −14% | −2% | |
| D114 | | | | 32% | 15% | |
| D113 | | | | −6% | 0% | |
| D90 | | | | 23% | 20% | |
| D103 | | | | 66% | 70% | |
| D102 | | | | 70% | 73% | |
| D85 | | | 17% | 34% | 30% | |
| D64 | | 26% | 62% | 79% | 83% | 77% |
| D66 | 8% | 8% | 40% | 55% | 71% | 58% |
| D86 | | | | 16% | 24% | |
| D63 | | | 34% | 55% | 68% | |
| D62 | | | 35% | 56% | 75% | |
| D83 | | | 2% | 25% | 39% | |
| D61 | | | 28% | 55% | 52% | |
| D82 | | | −1% | 28% | 57% | |
| D60 | | | −3% | 13% | 69% | |
| D81 | | | −3% | 1% | 5% | |
| D59 | | | 33% | 18% | 53% | |
| D80 | | | 6% | 0% | 23% | |
| D58 | | | 7% | 41% | 68% | |
| D77 | | | −4% | −1% | −7% | |
| D56 | | | 25% | 52% | 64% | |

TABLE 4
Additional Degradation, Enzymatic, and Proliferation Data

| | | | Degradation | | | |
|---|---|---|---|---|---|---|
| | | Degradation | Assay - Full | | | GeoMean |
| | Degradation | Assay - 3 | Dose Response: | Enzymatic | GeoMean | IC50 (uM) |
| | Assay - 3 | point: | IC50 (uM) [cell | GRK2 IC50: | IC50 (UM) | PAXF1657 |
| | point: Conc | Degradation | line: PAXF1657, | Av IC50 | PAXF1657 | KO Clone |
| Compound | (uM) | (%) | time point: 24 h] | GRK2 nM | EV | #4 |
| D172 | 0.3 | −7.47 | | 104 | 14.1 | >30.0 |
| | 1 | −8.22 | | | | |
| | 3 | −15.9 | | | | |
| D173 | 0.3 | 52.5 | | 18.6 | 0.03 | 0.26 |
| | 1 | 48.5 | | | | |
| | 3 | 14.7 | | | | |
| D174 | 0.3 | 63.6 | | 12 | <458E−06 | >30.0 |
| | 1 | 50.2 | | | | |
| | 3 | 28.3 | | | | |
| D175 | 0.3 | 87.7 | | 10.3 | <458E−06 | >30.0 |
| | 1 | 89.2 | | | | |
| | 3 | 82.8 | | | | |
| D176 | 0.3 | 33 | | 24.1 | 8.02 | >30.0 |
| | 1 | 24.4 | | | | |
| | 3 | 10.7 | | | | |
| D177 | 0.3 | 13.2 | | 10.2 | 27 | >30.0 |
| | 1 | 20.9 | | | | |
| | 3 | 36.8 | | | | |
| D178 | 0.3 | 45 | | 11.6 | 0.31 | >30 |
| | 1 | 72 | | | | |
| | 3 | 84 | | | | |
| D179 | 0.3 | 58 | | 6.36 | 0.14 | >30 |
| | 1 | 82 | | | | |
| | 3 | 88 | | | | |
| D180 | 0.3 | 43 | | 7.31 | 0.82 | >30 |
| | 1 | 74 | | | | |
| | 3 | 85 | | | | |
| D181 | 0.3 | 55 | | 11.8 | 0.58 | >30 |
| | 1 | 78 | | | | |
| | 3 | 86 | | | | |
| D116 | 0.3 | 15 | | 6.6 | 1.88 | 7.48 |
| | 1 | 30 | | | | |
| | 3 | −40 | | | | |
| D117 | 0.3 | 12.3 | | >1.0E+03 | | |
| | 1 | −20.5 | | | | |
| | 3 | −4.97 | | | | |
| D118 | 0.3 | −13.1 | | >1.0E+03 | | |
| | 1 | 3.23 | | | | |
| | 3 | 5.18 | | | | |
| D119 | 0.3 | 90 | | 16.1 | 0.116 | 2.73 |
| | 1 | 93.7 | | | | |
| | 3 | 89 | | | | |
| D120 | 0.3 | 90.8 | | 9.55 | 0.04 | >30 |
| | 1 | 94.8 | | | | |
| | 3 | 89.6 | | | | |
| D121 | 0.3 | 7.97 | | 44.7 | 11.4 | >30.0 |
| | 1 | 24 | | | | |
| | 3 | 53.8 | | | | |
| D122 | 0.3 | 84.9 | | 12.3 | 0.10 | >30 |
| | 1 | 90.6 | | | | |
| | 3 | 92.5 | | | | |
| D123 | 0.3 | 81.6 | 0.03 | 6.56 | <458E−06 | >30 |
| | 1 | 85.2 | | | | |
| | 3 | 84 | | | | |
| D124 | 0.3 | 80.5 | 0.10 | 5.4 | 0.09 | >30.0 |
| | 1 | 90.7 | | | | |
| | 3 | 89.3 | | | | |
| D125 | 0.3 | 71.9 | | 1.13 | 0.11 | 26.6 |
| | 1 | 82.5 | | | | |
| | 3 | 76.4 | | | | |
| D126 | 0.3 | 93.2 | 0.04 | 4.56 | 0.13 | >30.0 |
| | 1 | 94.1 | | | | |
| | 3 | 88 | | | | |
| D127 | 0.3 | 81.4 | | 2.2 | 0.10 | >30.0 |
| | 1 | 82.1 | | | | |
| | 3 | 78.5 | | | | |
| D128 | 0.3 | −23.2 | | 16.3 | 0.80 | >30.0 |
| | 1 | 8.53 | | | | |
| | 3 | −38.6 | | | | |
| D129 | 0.3 | 79.8 | 0.08 | 27.9 | 0.10 | >30.0 |
| | 1 | 77.5 | | | | |
| | 3 | 70.6 | | | | |
| D130 | 0.3 | 78.7 | 0.29 | 17.9 | 0.88 | 24.68 |
| | 1 | 84.7 | | | | |
| | 3 | 90.7 | | | | |
| D131 | 0.3 | 17.5 | 0.24 | 26.6 | 0.22 | >30.0 |
| | 1 | 68.7 | | | | |
| | 3 | 89.2 | | | | |
| D132 | 0.3 | 87.4 | 0.12 | 16.3 | 0.04 | >30.0 |
| | 1 | 92.4 | | | | |
| | 3 | 91.5 | | | | |
| D133 | 0.3 | 57.7 | | 9.05 | 0.71 | >30.0 |
| | 1 | 82.4 | | | | |
| | 3 | 86.7 | | | | |
| D134 | 0.3 | 64.1 | 0.29 | 29.9 | | |
| | 1 | 82.6 | | | | |
| | 3 | 86.7 | | | | |
| D135 | 0.3 | 82.2 | 0.06 | 23.7 | | |
| | 1 | 84.2 | | | | |
| | 3 | 84.3 | | | | |
| D136 | 0.3 | 87.3 | 0.218 | 12.4 | 0.06 | 5.09 |
| | 1 | 94.6 | | | | |
| | 3 | 91.6 | | | | |
| D137 | 0.3 | 71.2 | | 12.5 | 0.14 | 22.78 |
| | 1 | 79.2 | | | | |
| | 3 | 80.4 | | | | |
| D138 | 0.3 | 85 | 0.137 | 20.2 | 0.66 | >30.0 |
| | 1 | 92 | | | | |
| | 3 | 95 | | | | |
| D139 | 0.3 | 86 | 0.23 | 13.6 | 0.20 | 26.55 |
| | 1 | 92 | | | | |
| | 3 | 95 | | | | |
| D140 | 0.3 | 88 | 0.12 | 20.9 | 0.78 | 19.9 |
| | 1 | 92 | | | | |
| | 3 | 93 | | | | |
| D141 | 0.3 | 89 | 8.71 | 19.5 | 0.17 | 1.66 |
| | 1 | 88 | | | | |
| | 3 | 67 | | | | |
| D142 | 0.3 | 87.6 | 0.07 | 10.2 | 1.45 | >30.0 |
| | 1 | 87.5 | | | | |
| | 3 | 80.9 | | | | |
| D143 | 0.3 | 88 | 0.11 | 10.4 | 0.11 | 5.32 |
| | 1 | 82 | | | | |
| | 3 | 83 | | | | |
| D147 | 0.3 | 73.8 | | 10.1 | 0.11 | >30.0 |
| | 1 | 81.6 | | | | |
| | 3 | 83.1 | | | | |
| D148 | 0.3 | 46.7 | 2.57 | 14.1 | 0.10 | 8.31 |
| | 1 | 21.2 | | | | |
| | 3 | 0.218 | | | | |
| D149 | 0.3 | 62.1 | | 18.1 | 0.06 | 4.09 |
| | 1 | 61.1 | | | | |
| | 3 | 65.8 | | | | |
| D150 | 0.3 | 44 | | >1.00E+03 | 27.7 | >30.0 |
| | 1 | 43 | | | | |
| | 3 | 40 | | | | |
| D151 | 0.3 | 56 | | >1.00E+03 | 0.30 | 9.65 |
| | 1 | 58 | | | | |
| | 3 | 34 | | | | |
| D152 | 0.3 | 24 | | >1.00E+03 | 12.7 | >30.0 |
| | 1 | 28 | | | | |
| | 3 | 38 | | | | |
| D153 | 0.3 | 10 | | 125 | 0.34 | 6.82 |
| | 1 | 12 | | | | |
| | 3 | 18 | | | | |
| D154 | 0.3 | 25.1 | | 183 | 4.39 | >30.0 |
| | 1 | 19.5 | | | | |
| | 3 | 9.15 | | | | |
| D155 | 0.3 | 61.9 | | | >30.0 | >30.0 |
| | 1 | 58.1 | | | | |
| | 3 | −21.9 | | | | |
| D156 | 0.3 | −8.15 | | | 2.15 | >30.0 |
| | 1 | 2.96 | | | | |
| | 3 | 11.5 | | | | |
| D157 | 0.3 | 13.5 | | | 7.39 | >30.0 |
| | 1 | 11.1 | | | | |
| | 3 | 42.3 | | | | |
| D158 | 0.3 | 42.9 | | | 1.71 | >30.0 |
| | 1 | 65.8 | | | | |
| | 3 | 81.9 | | | | |
| D159 | 0.3 | 10 | | | 18.2 | >30.0 |
| | 1 | 46.3 | | | | |
| | 3 | 64.5 | | | | |
| D160 | 0.3 | 92 | | 25 | 0.05 | >30.0 |
| | 1 | 87 | | | | |
| | 3 | 83 | | | | |
| D105 | 0.3 | 87 | | 8.71 | 0.11 | 3.72 |
| | 1 | 86 | | | | |
| | 3 | 85 | | | | |
| D161 | 0.3 | 15 | | | 1.56 | 7.51 |
| | 1 | 23 | | | | |
| | 3 | 30 | | | | |
| D162 | 0.3 | 51 | | 14.1 | 0.49 | 11.39 |
| | 1 | 72.7 | | | | |
| | 3 | 72.9 | | | | |
| D163 | 0.3 | 74.4 | | 16.6 | 0.37 | >30.0 |
| | 1 | 81.5 | | | | |
| | 3 | 89.6 | | | | |
| D164 | 0.3 | 17.1 | | | 5.09 | >30.0 |
| | 1 | 14.1 | | | | |
| | 3 | 42.6 | | | | |
| D165 | 0.3 | −1.61 | | | 2.6 | >30.0 |
| | 1 | 35.5 | | | | |
| | 3 | 70.1 | | | | |
| D166 | 0.3 | 12.1 | | | 1.91 | >30.0 |
| | 1 | 32.4 | | | | |
| | 3 | 68.1 | | | | |
| D167 | 0.3 | 33.3 | | | 2.44 | >30.0 |
| | 1 | 57.4 | | | | |
| | 3 | 55.2 | | | | |
| D168 | 0.3 | 0.276 | | | >1.88 | 6.03 |
| | 1 | −12 | | | | |
| | 3 | −22 | | | | |
| D169 | 0.3 | 23.6 | | 14.5 | 0.12 | 8.15 |
| | 1 | 39.4 | | | | |
| | 3 | 24.0 | | | | |
| D170 | 0.3 | 51.6 | | 12.1 | 0.41 | >30.0 |
| | 1 | 72.9 | | | | |
| | 3 | 81.5 | | | | |
| D171 | 0.3 | 89.7 | | 28.7 | 0.09 | >30.0 |
| | 1 | 90.6 | | | | |
| | 3 | 76.2 | | | | |
| D198 | 0.3 | 83.9 | | 22.1 | 0.01 | >30.0 |
| | 1 | 81.6 | | | | |
| | 3 | 50.3 | | | | |
| D197 | 0.3 | −6.8 | | 82.3 | 5.89 | >30.0 |
| | 1 | 27.7 | | | | |
| | 3 | 54.5 | | | | |
| D196 | 0.3 | 66.2 | | 23.8 | 0.36 | >30.0 |
| | 1 | 71.1 | | | | |
| | 3 | 38.9 | | | | |
| D195 | 0.3 | −46.4 | | | 1.09 | >30.0 |
| | 1 | 9.8 | | | | |
| | 3 | 32.1 | | | | |
| D194 | 0.3 | 25 | | | | |
| | 1 | 15 | | | | |
| | 3 | 0 | | | | |
| D193 | 0.3 | 19.3 | | | | |
| | 1 | 16.7 | | | | |
| | 3 | 29.6 | | | | |
| D192 | 0.3 | 13.5 | | | | |
| | 1 | 37.4 | | | | |
| | 3 | 56.2 | | | | |
| D191 | 0.3 | 36.7 | | 24.9 | 0.32 | >30.0 |
| | 1 | 78.1 | | | | |
| | 3 | 86.0 | | | | |
| D190 | 0.3 | −22 | | 12.7 | 1.74 | 26.8 |
| | 1 | 51 | | | | |
| | 3 | 53 | | | | |
| D189 | 0.3 | 2.78 | | | 0.42 | >30.0 |
| | 1 | 35.8 | | | | |
| | 3 | 58.5 | | | | |
| D188 | 0.3 | 56.9 | | | 1.26 | >30.0 |
| | 1 | 67.7 | | | | |
| | 3 | 78.2 | | | | |
| D187 | 0.3 | 11.8 | | | 2.09 | >30.0 |
| | 1 | 23.8 | | | | |
| | 3 | 63.1 | | | | |
| D186 | 0.3 | 20 | | | 0.56 | 5.09 |
| | 1 | 36.3 | | | | |
| | 3 | −32.6 | | | | |
| D185 | 0.3 | 1.79 | | 211 | >30 | >30 |
| | 1 | 17.5 | | | | |
| | 3 | 19.2 | | | | |
| D184 | 0.3 | 1.96 | | | | |
| | 1 | 17.7 | | | | |
| | 3 | 20.6 | | | | |
| D183 | 0.3 | 82 | | 16.7 | 0.11 | >30 |
| | 1 | 86.5 | | | | |
| | 3 | 94.6 | | | | |
| D182 | 0.3 | 29.1 | 0.38 | | 0.12 | 10 |
| | 1 | 29.5 | | | | |
| | 3 | 21.3 | | | | |
| D109 | 0.3 | 26.1 | | 54.9 | 0.68 | 5.17 |
| | 1 | 45.9 | | | | |
| | 3 | 66.6 | | | | |

### Synthesis of Compounds

The examples below include procedures, intermediates, and characterization data useful, e.g., for the preparation of compounds provided herein. All synthetic steps, procedures, compounds (e.g., synthetic intermediates), reaction conditions, reaction mixtures, reagents, etc. are included herein as aspects of the present disclosure.

### Example 6: Preparation of Compounds D64, D85, D57, D79, D55, D78, D108, D109, D180, D181, D194, D191, D190, D189, D185, D184, D183, and D182

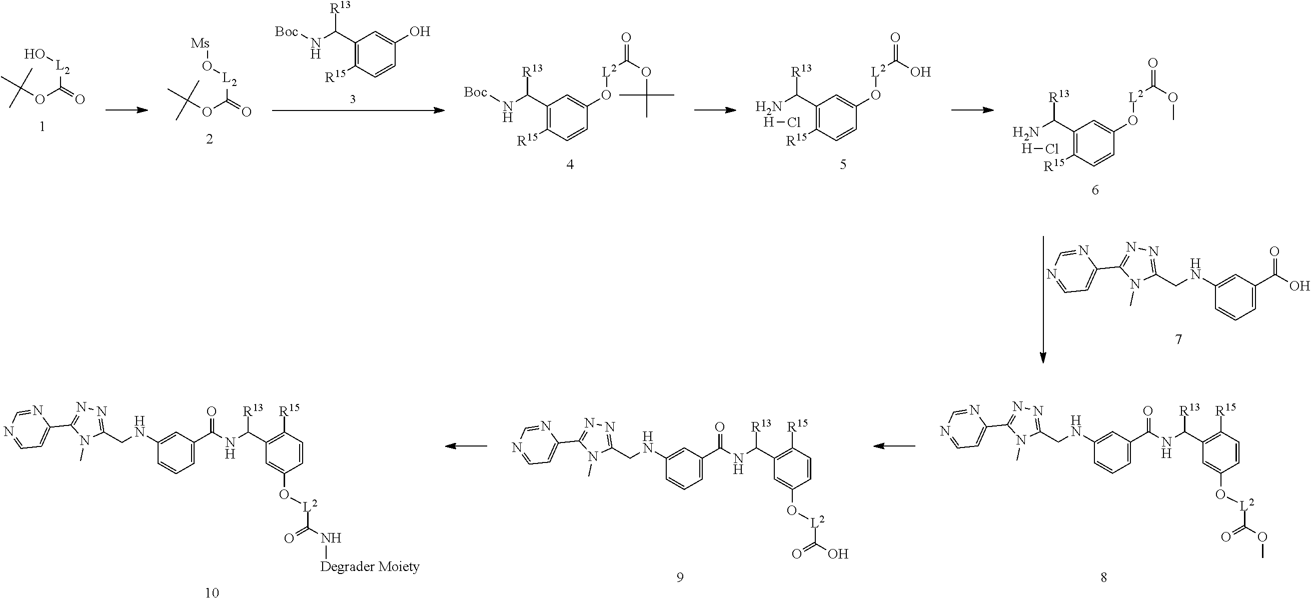

### General LCMS Conditions

C18 column 100×4.6 mm, 5.0 μm, pore size 100 Å, water-acetonitrile+0.1% TFA, gradient 5 to 87% for 10 min.

C18 column 20×2 mm, particle size 2.5 μm, pore size 100 Å, water-acetonitrile+0.1% TFA, gradient 5 to 87% for 3 min.

HPLC neutral conditions (YMC-Pack ODS-AQ 250×20 mml, S-10 μm, 12 nm, gradient water-acetonitrile).

HPLC standard acidic conditions (YMC-Pack ODS-AQ 250×20 mml, S-10 μm, 12 nm, gradient A solution-B solution (A: 1000 mL H₂O-226 μl TFA, B: 1000 mL CH₃CN-226 μL TFA)

### General Procedures

Compound 2. A solution of compound 1 (2.9 mmol, 1 eq) and diisopropylamine (0.56 g, 0.75 mL, 4.35 mmol, 1.5 eq) in dry DCM (50 mL) was cooled to 0° C., and mesyl chloride (0.40 g, 0.27 mL, 3.48 mmol, 1.2 eq) was added. The mixture was warmed to 0° C. over 1 h period, stirred for 1 h. A progress of the reaction was monitored by TLC. The mixture was washed by water (3*20 mL), dried over Na₂SO₄, and evaporated. The product immediately was used in the next step without additional purification.

Compound 4. Compound 3 (2.5 mmol, 1 eq), compound 2 (2.75 mmol, 1.1 eq), K₂CO₃ (5.0 mmol, 2 eq) in dry acetonitrile (50 mL) were stirred for 12 hours at reflux. The precipitate was filtered off, washed with acetonitrile. The solvent was evaporated to dryness to give the product. The product 4 was used in the next step without additional purification.

Compound 5. A solution of compound 4 (2.5 mmol, 1 eq) in dry DCM (50 mL) with 12.5 mL of 3 M HCl in dioxane (5 eq) were stirred for 24 h at r.t. The mixture was evaporated to dryness to give the crude product 5 which was used in the next step without additional purification.

Compound 6. To a solution of compound 5 (2.5 mmol, 1 eq) in dry MeOH (20 mL) was added dropwise SOCl₂ (0.36 mL, 5.0 mmol, 2 eq). The mixture was stirred for 12 hours at r.t. and then evaporated to dryness to give the crude product 5 which no need additional purification.

Compound 8. Compound 6 (1.2 mmol, 1 eq), compound 7 (1.2 mmol, 1 eq), HOBt (1.2 mmol, 1 eq), EDCI (1.32 mmol, 1.1 eq) and DIPEA (4.8 mmol, 4 eq) in dry DCM (30 mL) were stirred for 12 h at r.t. The reaction mixture was washed with water (3*10 mL), brine (2×10 mL), dried on activated Na₂SO₄, and concentrated in a rotary evaporator under reduced pressure at ambient temperature. The residue was purified by silica gel flash column chromatography using chloroform and methanol as an eluent to afford the desired product 8.

Compound 9. To a solution of compound 8 (1.2 mmol, 1 eq) in MeOH (20 mL) was added a solution KOH (3.6 mmol, 3 eq) in 3 mL H₂O. The mixture was stirred for 12-36 h at r.t. and then MeOH evaporated. To the residue was added 15 mL of H₂O and the mixture was acidified to pH=5 with 1N aq. HCl and aqueous phase was extracted with 4*15 mL of DCM. Combined organic phases were washed with brine, dried over Na₂SO₄, and evaporated. The product was used in the next step without additional purification.

Compound 10. Compound 9 (0.1 mmol, 1 eq), recruiter (0.1 mmol, 1 eq), TBTU (0.2 mmol, 2 eq) and DIPEA (0.4 mmol, 4 eq) in dry pyridine (3 mL) were stirred for 12 hours at r.t. The mixture was evaporated to dryness. The residue was purified by HPLC in neutral conditions (YMC-Pack ODS-AQ 250×20 ml, S-10 μm, 12 nm, gradient water-acetonitrile).

### Compounds and Analytical Data

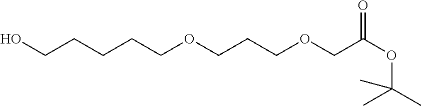 [SMILES: CC(C)(C)OC(=O)COCCCOCCCCCO]

Tert-butyl 2-(3-((5-hydroxypentyl)oxy)propoxy)acetate was prepared according to the procedures ACS Med. Chem. Lett. 2020, 11, 8, 1539-1547.

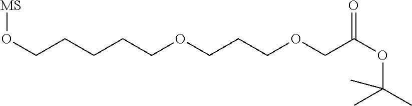

Compound 2. Tert-butyl 3,9,13-trioxa-2-thiapentadecan-15-oate 2,2-dioxide. Colorless oil. Yield 95%.

The product was used in the next step immediately.

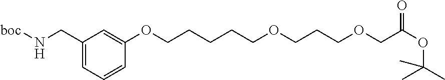

Compound 4. Tert-butyl 2-(3-((5-(3-(((tert-butoxycarbonyl)amino)methyl)phenoxy) pentyl)oxy)propoxy)acetate. Yellowish oil. Yield 99%. LCMS (C18 column 20×2 mm, 2.5 μm, pore size 100 Å, water-acetonitrile+0.1% TFA, gradient 5 to 87% for 3 min, retention time 2.04 min). MS (ESI) m/z 482.5 [MH]⁺. ¹H NMR (400 MHz, DMSO-d₆) δ 7.34 (t, J=6.36 Hz, 1H), 7.19 (t, J=7.95, 1H), 6.80-6.74 (m, 3H), 4.08 (d, J=5.99 Hz, 2H), 3.94-3.91 (m, 4H), 1.75-1.66 (m, 4H), 1.57-1.51 (m, 2H), 1.47-1.39 (m, 20H).

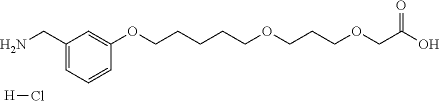 [SMILES: NCc1cccc(OCCCCCOCCCOCC(=O)O)c1 ; Cl]

Compound 5. 2-(3-((5-(3-(aminomethyl)phenoxy)pentyl)oxy)propoxy)acetic acid hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 0.98 min). MS (ESI) m/z 326.5 [MH]⁺.

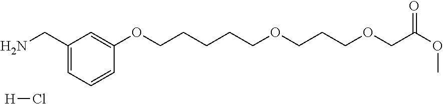 [SMILES: COC(=O)COCCCOCCCCCOc1cccc(CN)c1 ; Cl]

Compound 6. methyl 2-(3-((5-(3-(aminomethyl)phenoxy)pentyl)oxy)propoxy)acetate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.22 min). MS (ESI) m/z 340.5 [MH]⁺.

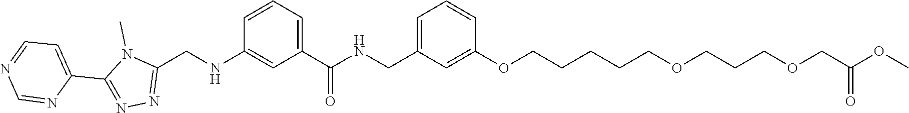 [SMILES: COC(=O)COCCCOCCCCCOc1cccc(CNC(=O)c2cccc(NCc3nnc(-c4ccncn4)n3C)c2)c1]

Compound 8. methyl 2-(3-((5-(3-((3-(((4-methyl-5-(pyrimidin-4-yl)-4H-1,2,4-triazol-3-yl)methyl)amino)benzamido)methyl)phenoxy)pentyl)oxy)propoxy)acetate. Light brown solid. Yield 98%. LCMS (C18 column 20×2 mm, 2.5 μm, pore size 100 Å, water-acetonitrile+0.1% TFA, gradient 5 to 87% for 3 min, retention time 1.39 min). MS (ESI) m/z 632.8 [MH]⁺. ¹H NMR (400 MHz, DMSO-d₆) δ 9.33 (s, 1H), 8.97 (d, J=5.26, 1H), 8.81 (t, J=587 Hz, 1H), 8.18 (dd, J=5.26, 0.98 Hz, 1H), 7.24-7.09 (m, 4H), 6.92-6.77 (m, 4H), 6.47 (t, J=5.38 Hz, 1H), 4.58 (d, J=5.50 Hz, 2H), 4.42 (d, J=5.87 Hz, 2H), 4.87 (br.s, 5H),), 3.92 (t, J=6.36 Hz, 2H), 3.66-3.63 (m, 4H), 3.51-3.47 (m, 2H), 3.41-3.36 (m, 3H), 1.75-1.66 (m, 4H), 1.56-1.38 (m, 4H).

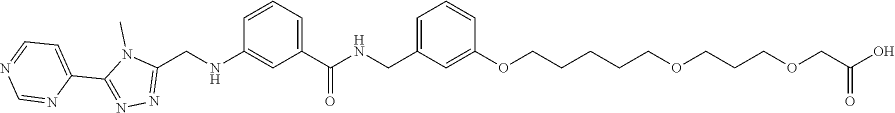 [SMILES: Cn1c(CNc2cccc(C(=O)NCc3cccc(OCCCCCOCCCOCC(=O)O)c3)c2)nnc1-c1ccncn1]

Compound 9. 2-(3-((5-(3-((3-(((4-methyl-5-(pyrimidin-4-yl)-4H-1,2,4-triazol-3-yl)methyl)amino)benzamido)methyl)phenoxy)pentyl)oxy)propoxy)acetic acid. Light brown solid. Yield 71%. LCMS (C18 column 20×2 mm, 2.5 μm, pore size 100 Å, water-acetonitrile+0.1% TFA, gradient 5 to 87% for 3 min, retention time 1.26 min). MS (ESI) m/z 618.7 [MH]⁺.

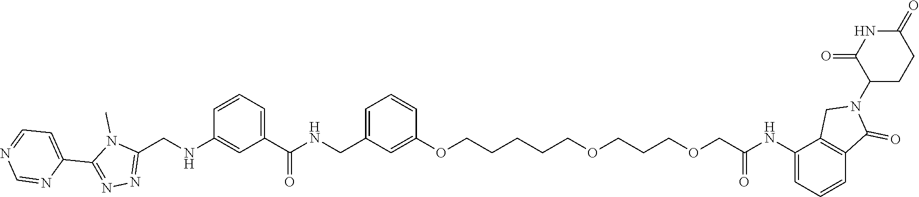 [SMILES: Cn1c(CNc2cccc(C(=O)NCc3cccc(OCCCCCOCCCOCC(=O)Nc4cccc5c4CN(C4CCC(=O)NC4=O)C5=O)c3)c2)nnc1-c1ccncn1]

Compound 10. D64. N-(3-((5-(3-(2-((2-(2,6-dioxopiperidin-3-yl)-1-oxoisoindolin-4-yl)amino)-2-oxoethoxy)propoxy)pentyl)oxy)benzyl)-3-(((4-methyl-5-(pyrimidin-4-yl)-4H-1,2,4-triazol-3-yl)methyl)amino)benzamide. Colorless solid. LCMS (C18 column 100×4.6 mm, 5.0 μm, pore size 100 Å, water-acetonitrile+0.1% TFA, gradient 5 to 87% for 10 min, retention time 5.61 min). MS (ESI) m/z 860.0 [MH]⁺. ¹H NMR (400 MHz, DMSO-d₆). δ 10.99 (s, 1H), 9.69 (s, 1H), 9.33 (d, J=1.3 Hz, 1H), 8.97 (d, J=5.3 Hz, 1H), 8.81 (t, J=6.0 Hz, 1H), 8.17 (dd, J=5.3, 1.4 Hz, 1H), 7.73 (d, J=6.9 Hz, 1H), 7.59-7.43 (m, 2H), 7.27-7.05 (m, 4H), 6.95-6.72 (m, 4H), 6.47 (t, J=5.87 Hz, 1H), 5.13 (dd, J=13.3, 5.1 Hz, 1H), 4.57 (d, J=5.5 Hz, 2H), 4.46-4.33 (m, Hz, 4H), 4.10-4.00 (m, 5H), 3.90 (t, J=6.6 Hz, 2H), 3.58 (t, J=6.4 Hz, 2H), 3.45 (t, J=6.5 Hz, 2H), 3.35 (t, J=6.3 Hz, 2H), 2.91 (s, 1H), 2.72-2.55 (m, 1H), 2.35 (d, J=17.6 Hz, 1H), 2.02 (s, 1H), 1.87-1.74 (m, 2H), 1.72-1.62 (m, 2H), 1.45 (dd, J=37.6, 6.9 Hz, 4H).

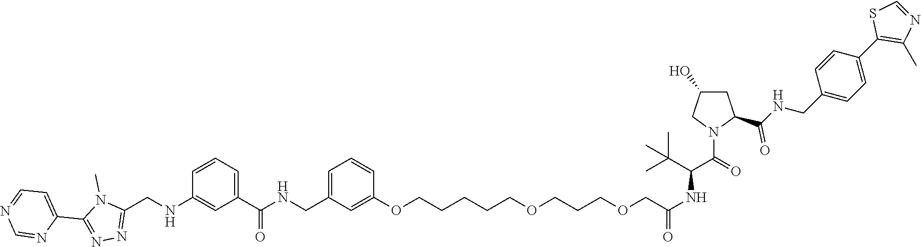 [SMILES: Cc1ncsc1-c1ccc(CNC(=O)[C@@H]2C[C@@H](O)CN2C(=O)[C@@H](NC(=O)COCCCOCCCCCOc2cccc(CNC(=O)c3cccc(NCc4nnc(-c5ccncn5)n4C)c3)c2)C(C)(C)C)cc1]

Compound 10. D85. (2S,4R)-1-((S)-3,3-dimethyl-2-(2-(3-((5-(3-((3-(((4-methyl-5-(pyrimidin-4-yl)-4H-1,2,4-triazol-3-yl)methyl)amino)benzamido)methyl)phenoxy)pentyl)oxy)propoxy)acetamido)butanoyl)-4-hydroxy-N-(4-(4-methylthiazol-5-yl)benzyl)pyrrolidine-2-carboxamide. Colorless solid. LCMS (C18 column 100×4.6 mm, 5.0 μm, pore size 100 Å, water-acetonitrile+0.1% TFA, gradient 5 to 87% for 10 min, retention time 6.11 min). MS (ESI) m/z 1030.0 [MH]⁺. ¹H NMR (400 MHz, DMSO-d₆). δ 9.33 (d, J=1.3 Hz, 1H), 8.97 (t, J=2.6 Hz, 2H), 8.80 (t, J=5.75 Hz, 1H), 8.59 (t, J=5.62 Hz, 1H), 8.17 (dd, J=5.1, 1.3 Hz, 1H), 7.48-7.30 (m, 5H), 7.27-7.06 (m, 4H), 6.94-6.73 (m, 4H), 6.47 (t, J=6.5, 1H), 5.14 (d, J=3.5 Hz, 1H), 4.63-4.51 (m, 3H), 4.48-4.18 (m, 6H), 4.07 (s, 3H), 3.96-3.82 (m, 4H), 3.69-3.56 (m, J=16.2 Hz, 2H), 3.52 (t, J=6.7 Hz, 2H), 3.42 (t, J=6.3 Hz, 2H), 3.38-3.33 (m, 2H), 2.43 (s, 3H), 2.12-2.00 (m, 1H), 1.94-1.84 (m, 1H), 1.80-1.61 (m, 4H), 1.54-1.32 (m, 4H), 0.93 (s, 9H).

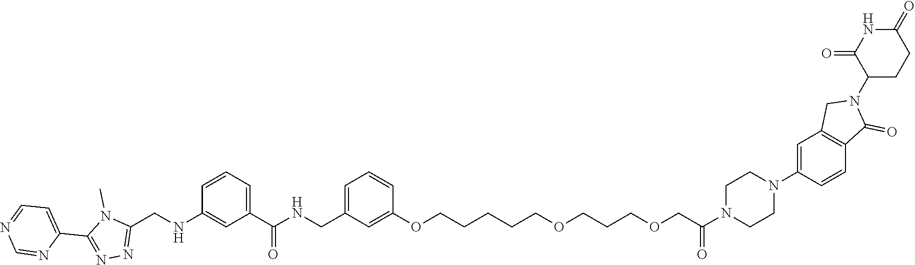 [SMILES: Cn1c(CNc2cccc(C(=O)NCc3cccc(OCCCCCOCCCOCC(=O)N4CCN(c5ccc6c(c5)CN(C5CCC(=O)NC5=O)C6=O)CC4)c3)c2)nnc1-c1ccncn1]

Compound 10. D189. N-(3-((5-(3-(2-(4-(2-(2,6-dioxopiperidin-3-yl)-1-oxoisoindolin-5-yl)piperazin-1-yl)-2-oxoethoxy)propoxy)pentyl)oxy)benzyl)-3-(((4-methyl-5-(pyrimidin-4-yl)-4H-1,2,4-triazol-3-yl)methyl)amino)benzamide. Colorless solid. Yield 62%. LCMS (C18 column 100×4.6 mm, 5.0 μm, pore size 100 Å, water-acetonitrile+0.1% TFA, gradient 5 to 87% for 10 min, retention time 5.36 min). MS (ESI) m/z 928.7 [MH]⁺. ¹H NMR (400 MHz, DMSO-d₆) δ 10.94 (s, 1H), 9.33 (d, J=1.4 Hz, 1H), 8.97 (d, J=5.3 Hz, 1H), 8.81 (t, J=6.1 Hz, 1H), 8.17 (dd, J=5.3, 1.4 Hz, 1H), 7.53 (d, J=8.5 Hz, 1H), 7.24 (m, 1H), 7.19 (m, 2H), 7.08 (m, 3H), 6.90 (dd, J=7.9, 2.4 Hz, 1H), 6.85 (m, 2H), 6.77 (m, 1H), 6.48 (t, J=5.7 Hz, 1H), 5.04 (dd, J=13.3, 5.1 Hz, 1H), 4.57 (d, J=5.7 Hz, 2H), 4.40 (d, J=6.0 Hz, 2H), 4.32 (d, J=16.9 Hz, 1H), 4.20 (d, J=16.9 Hz, 1H), 4.15 (s, 2H), 4.07 (s, 3H), 3.90 (t, J=6.4 Hz, 2H), 3.57 (s, 4H), 3.47 (t, J=6.4 Hz, 2H), 3.40 (t, J=6.3 Hz, 2H), 3.34 (t, J=6.3 Hz, 2H), 3.28 (m, 4H), 2.89 (m, 1H), 2.63 (m, 1H), 2.35 (m, 1H), 1.94 (m, 1H), 1.70 (m, 4H), 1.51 (p, J=6.5 Hz, 2H), 1.40 (m, 2H).

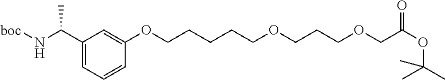

Compound 4. (R)-tert-butyl 2-(3-((5-(3-(1-((tert butoxycarbonyl)amino)ethyl)phenoxy) pentyl) oxy)propoxy)acetate. Yellowish oil. Yield 98%. LCMS (C18 column 20×2 mm, 2.5 μm, pore size 100 Å, water-acetonitrile+0.1% TFA, gradient 5 to 87% for 3 min, retention time 2.13 min). MS (ESI) m/z 496.9 [MH]⁺.

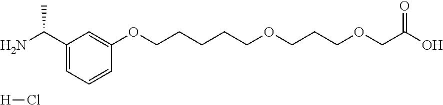 [SMILES: C[C@@H](N)c1cccc(OCCCCCOCCCOCC(=O)O)c1 ; Cl]

Compound 5. (R)-2-(3-((5-(3-(1-aminoethyl)phenoxy)pentyl)oxy)propoxy)acetic acid hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.00 min). MS (ESI) m/z 340.4 [MH]⁺.

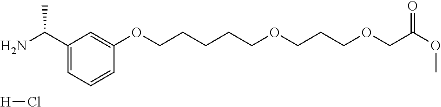 [SMILES: COC(=O)COCCCOCCCCCOc1cccc([C@@H](C)N)c1 ; Cl]

Compound 6. (R)-methyl 2-(3-((5-(3-(1-aminoethyl)phenoxy)pentyl)oxy)propoxy)acetate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.14 min). MS (ESI) m/z 354.5 [MH]⁺.

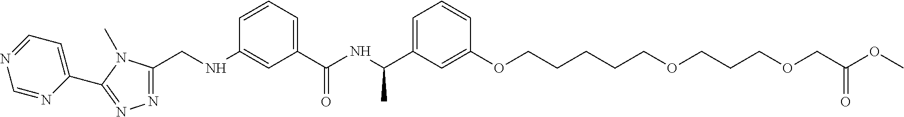 [SMILES: COC(=O)COCCCOCCCCCOc1cccc([C@@H](C)NC(=O)c2cccc(NCc3nnc(-c4ccncn4)n3C)c2)c1]

Compound 8. (R)-methyl 2-(3-((5-(3-(1-(3-(((4-methyl-5-(pyrimidin-4-yl)-4H-1,2,4-triazol-3-yl)methyl)amino)benzamido)ethyl)phenoxy)pentyl)oxy)propoxy)acetate. Light brown solid. Yield 88%. LCMS (C18 column 20×2 mm, 2.5 μm, pore size 100 Å, water-acetonitrile+0.1% TFA, gradient 5 to 87% for 3 min, retention time 1.51 min). MS (ESI) m/z 646.8 [MH]⁺.

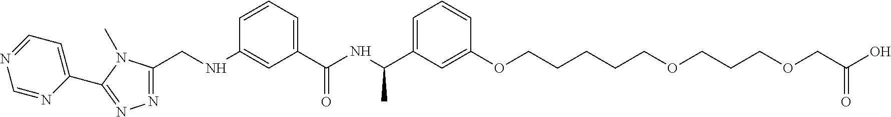 [SMILES: C[C@@H](NC(=O)c1cccc(NCc2nnc(-c3ccncn3)n2C)c1)c1cccc(OCCCCCOCCCOCC(=O)O)c1]

Compound 9. (R)-2-(3-((5-(3-(1-(3-(((4-methyl-5-(pyrimidin-4-yl)-4H-1,2,4-triazol-3-yl)methyl)amino)benzamido)ethyl)phenoxy)pentyl)oxy)propoxy)acetic acid. Light brown solid. Yield 75%. LCMS (C18 column 20×2 mm, 2.5 μm, pore size 100 Å, water-acetonitrile+0.1% TFA, gradient 5 to 87% for 3 min, retention time 1.41 min). MS (ESI) m/z 632.8 [MH]⁺.

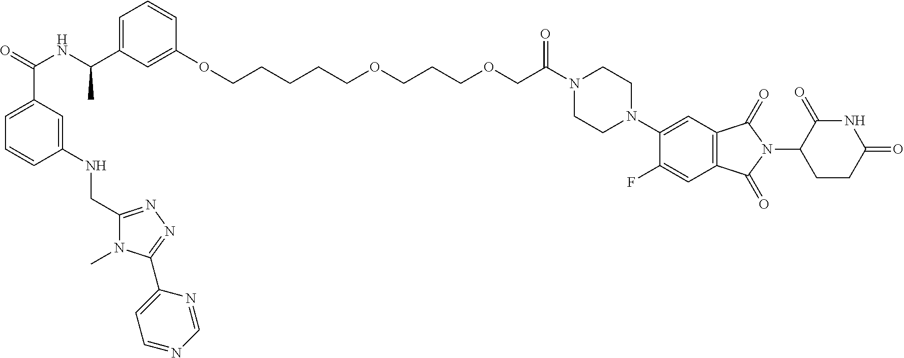 [SMILES: C[C@@H](NC(=O)c1cccc(NCc2nnc(-c3ccncn3)n2C)c1)c1cccc(OCCCCCOCCCOCC(=O)N2CCN(c3cc4c(cc3F)C(=O)N(C3CCC(=O)NC3=O)C4=O)CC2)c1]

Compound 10. D180. N-((1R)-1-(3-((5-(3-(2-(4-(2-(2,6-dioxopiperidin-3-yl)-6-fluoro-1,3-dioxoisoindolin-5-yl)piperazin-1-yl)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 79%. LCMS (C18 column 100×4.6 mm, 5.0 μm, pore size 100 Å, water-acetonitrile+0.1% TFA, gradient 5 to 87% for 10 min, retention time 6.19 min). MS (ESI) m/z 975.2 [MH]⁺. ¹H NMR (400 MHz, DMSO-d₆) δ 11.09 (s, 1H), 9.32 (d, J=1.4 Hz, 1H), 8.96 (d, J=5.3 Hz, 1H), 8.54 (d, J=8.2 Hz, 1H), 8.17 (dd, J=5.3, 1.4 Hz, 1H), 7.74 (d, J=11.2 Hz, 1H), 7.46 (d, J=7.3 Hz, 1H), 7.18 (m, 3H), 7.09 (d, J=7.6 Hz, 1H), 6.91 (m, 3H), 6.75 (m, 1H), 6.44 (t, J=5.7 Hz, 1H), 5.09 (m, 2H), 4.57 (d, J=5.7 Hz, 2H), 4.14 (s, 2H), 4.07 (s, 3H), 3.92 (t, J=6.4 Hz, 2H), 3.59 (s, 4H), 3.48 (t, J=6.4 Hz, 2H), 3.41 (t, J=6.4 Hz, 2H), 3.35 (t, J=6.4 Hz, 2H), 3.25 (s, 4H), 2.86 (m, 1H), 2.59 (m, 1H), 2.51 (m, 1H), 2.03 (m, 1H), 1.72 (m, 4H), 1.53 (m, 2H), 1.43 (m, 5H).

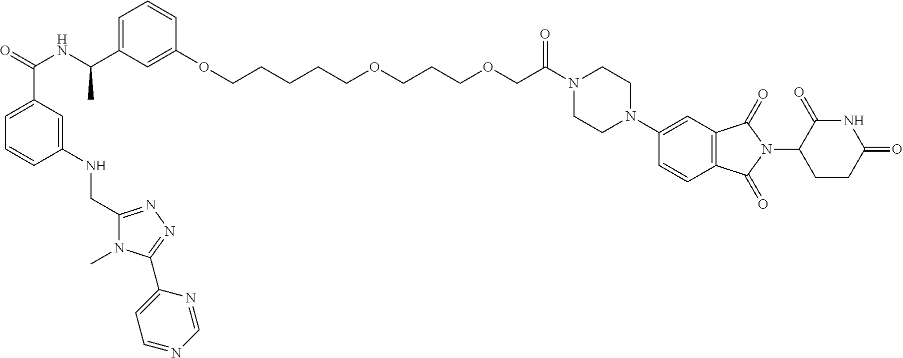 [SMILES: C[C@@H](NC(=O)c1cccc(NCc2nnc(-c3ccncn3)n2C)c1)c1cccc(OCCCCCOCCCOCC(=O)N2CCN(c3ccc4c(c3)C(=O)N(C3CCC(=O)NC3=O)C4=O)CC2)c1]

Compound 10. D181. N-((1R)-1-(3-((5-(3-(2-(4-(2-(2,6-dioxopiperidin-3-yl)-1-oxoisoindolin-5-yl)piperazin-1-yl)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 95%. LCMS (C18 column 100×4.6 mm, 5.0 μm, pore size 100 Å, water-acetonitrile+0.1% TFA, gradient 5 to 87% for 10 min, retention time 5.59 min). MS (ESI) m/z 942.8 [MH]⁺. ¹H NMR (400 MHz, DMSO-d₆) δ 10.92 (s, 1H), 9.32 (d, J=1.4 Hz, 1H), 8.96 (d, J=5.3 Hz, 1H), 8.54 (d, J=8.2 Hz, 1H), 8.17 (dd, J=5.3, 1.5 Hz, 1H), 7.53 (d, J=8.4 Hz, 1H), 7.18 (m, 3H), 7.10 (d, J=7.6 Hz, 1H), 7.06 (m, 2H), 6.91 (m, 3H), 6.75 (m, 1H), 6.44 (t, J=5.7 Hz, 1H), 5.06 (m, 2H), 4.57 (d, J=5.7 Hz, 2H), 4.26 (m, 2H), 4.14 (s, 2H), 4.06 (s, 3H), 3.91 (t, J=6.4 Hz, 2H), 3.57 (s, 4H), 3.48 (t, J=6.4 Hz, 2H), 3.40 (t, J=6.4 Hz, 2H), 3.35 (m, 6H), 2.88 (m, 1H), 2.63 (m, 1H), 2.35 (m, 1H), 1.94 (m, 1H), 1.71 (m, 4H), 1.51 (m, 2H), 1.42 (m, 5H).

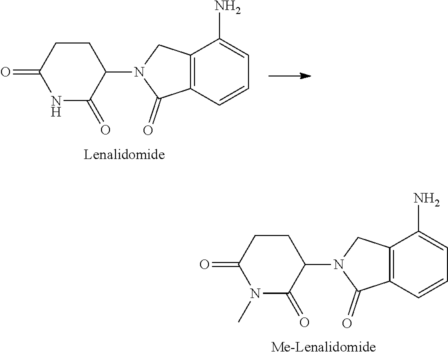 [SMILES: Nc1cccc2c1CN(C1CCC(=O)NC1=O)C2=O ; CN1C(=O)CCC(N2Cc3c(N)cccc3C2=O)C1=O]

Me-Lenalidomide. 3-(4-amino-1-oxoisoindolin-2-yl)-1-methylpiperidine-2,6-dione To a suspension of Lenalidomide (1.5 g, 5.8 mmol) in acetone (30 ml) K2CO3 (1.6 g, 11.6 mmol, 2 eq) was added followed by Mel (1.2 g, 8.7 mmol, 1.5 eq). The mixture was stirred at rt for 72 h. The precipitate was filtered off and washed with water to give Me-Lenalidomide as a white powder. Yield 1.0 g, 65%. ¹H NMR (400 MHz, DMSO-d₆) δ 7.19 (t, J=7.5 Hz, 1H), 6.92 (d, J=7.1 Hz, 1H), 6.80 (d, J=7.5 Hz, 1H), 5.41 (s, 2H), 5.16 (d, J=8.9 Hz, 1H), 4.15 (dd, J=44.7, 17.0 Hz, 2H), 3.12-2.90 (m, 4H), 2.86-2.70 (m, 1H), 2.41-2.21 (m, 1H), 2.14-1.94 (m, 1H).

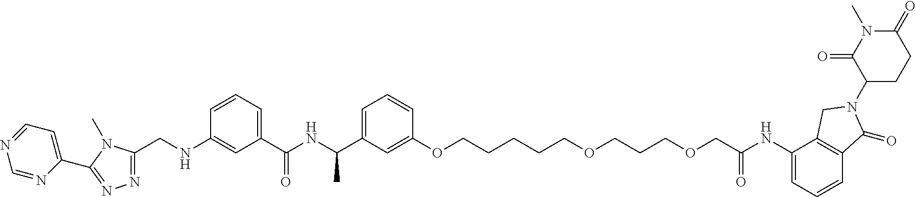 [SMILES: C[C@@H](NC(=O)c1cccc(NCc2nnc(-c3ccncn3)n2C)c1)c1cccc(OCCCCCOCCCOCC(=O)Nc2cccc3c2CN(C2CCC(=O)N(C)C2=O)C3=O)c1]

Compound 10. D194. N-((1R)-1-(3-((5-(3-(2-((2-(1-methyl-2,6-dioxopiperidin-3-yl)-1-oxoisoindolin-4-yl)amino)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 41%. LCMS (C18 column 100×4.6 mm, 5.0 μm, pore size 100 Å, water-acetonitrile+0.1% TFA, gradient 5 to 87% for 10 min, retention time 5.96 min). MS (ESI) m/z 887.8 [MH]⁺. ¹H NMR (400 MHz, DMSO-d₆) δ 9.69 (s, 1H), 9.33 (d, J=1.4 Hz, 1H), 8.96 (d, J=5.2 Hz, 1H), 8.55 (d, J=8.2 Hz, 1H), 8.17 (dd, J=5.3, 1.4 Hz, 1H), 7.73 (d, J=7.5 Hz, 1H), 7.56 (d, J=7.5 Hz, 1H), 7.50 (t, J=7.5 Hz, 1H), 7.18 (m, 3H), 7.10 (d, J=7.6 Hz, 1H), 6.91 (m, 3H), 6.75 (m, 1H), 6.45 (t, J=5.7 Hz, 1H), 5.20 (dd, J=13.4, 5.1 Hz, 1H), 5.09 (p, J=6.7 Hz, 1H), 4.57 (d, J=5.6 Hz, 2H), 4.34 (m, 2H), 4.07 (m, 5H), 3.91 (t, J=6.4 Hz, 2H), 3.57 (t, J=6.4 Hz, 2H), 3.45 (t, J=6.3 Hz, 2H), 3.35 (t, J=6.3 Hz, 2H), 3.00 (s, 3H), 2.97 (m, 1H), 2.75 (m, 1H), 2.36 (m, 1H), 2.01 (m, 1H), 1.80 (p, J=6.4 Hz, 2H), 1.67 (p, J=6.7 Hz, 2H), 1.51 (p, J=6.3 Hz, 2H), 1.42 (m, 5H).

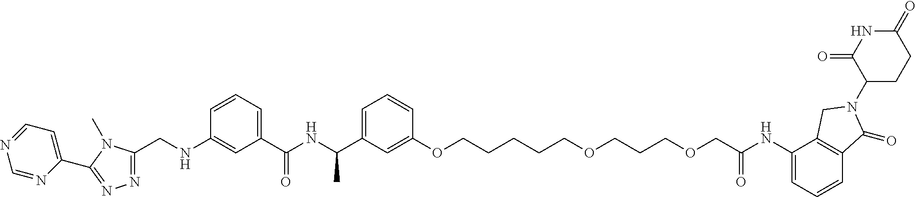 [SMILES: C[C@@H](NC(=O)c1cccc(NCc2nnc(-c3ccncn3)n2C)c1)c1cccc(OCCCCCOCCCOCC(=O)Nc2cccc3c2CN(C2CCC(=O)NC2=O)C3=O)c1]

Compound 10. D183. N-((1R)-1-(3-((5-(3-(2-((2-(2,6-dioxopiperidin-3-yl)-1-oxoisoindolin-4-yl)amino)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 41%. LCMS (C18 column 100×4.6 mm, 5.0 μm, pore size 100 Å, water-acetonitrile+0.1% TFA, gradient 5 to 87% for 10 min, retention time 5.85 min). MS (ESI) m/z 873.5 [MH]⁺. ¹H NMR (400 MHz, DMSO-d₆). δ 11.00 (s, 1H), 9.69 (s, 1H), 9.33 (d, J=1.3 Hz, 1H), 8.96 (d, J=5.3 Hz, 1H), 8.56 (d, J=8.5 Hz, 1H), 8.17 (dd, J=5.3, 1.4 Hz, 1H), 7.73 (d, J=7.9 Hz, 1H), 7.59-7.46 (m, 2H), 7.25-7.13 (m, 3H), 7.10 (d, J=7.7 Hz, 1H), 6.97-6.86 (m, 3H), 6.75 (d, J=8.0 Hz, 1H), 6.46 (t, J=5.14 Hz, 1H), 5.19-5.01 (m, 2H), 4.57 (d, J=5.6 Hz, 2H), 4.37 (d, J=9.0 Hz, 2H), 4.10-4.03 (m, 5H), 3.91 (t, J=6.2 Hz, 2H), 3.58 (t, J=6.5 Hz, 2H), 3.45 (t, J=6.3 Hz, 2H), 3.38-3.34 (m, 2H), 2.97-2.83 (m, 1H), 2.69-2.55 (m, 1H), 2.42-2.28 (m, 1H), 2.07-1.94 (m, 1H), 1.87-1.74 (m, 2H), 1.73-1.61 (m, 2H), 1.57-1.34 (m, 7H).

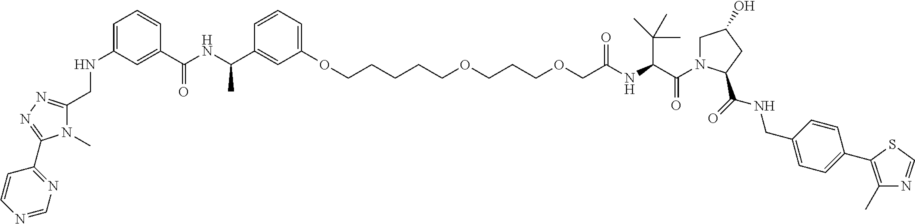 [SMILES: Cc1ncsc1-c1ccc(CNC(=O)[C@@H]2C[C@@H](O)CN2C(=O)[C@@H](NC(=O)COCCCOCCCCCOc2cccc([C@@H](C)NC(=O)c3cccc(NCc4nnc(-c5ccncn5)n4C)c3)c2)C(C)(C)C)cc1]

Compound 10. D182. (2S,4R)-1-((S)-3,3-dimethyl-2-(2-(3-((5-(3-((R)-1-(3-(((4-methyl-5-(pyrimidin-4-yl)-4H-1,2,4-triazol-3-yl)methyl)amino)benzamido)ethyl) phenoxy)pentyl)oxy)propoxy)acetamido)butanoyl)-4-hydroxy-N-(4-(4-methylthiazol-5-yl)benzyl)pyrrolidine-2-carboxamide. Colorless solid. Yield 42%. LCMS (C18 column 100×4.6 mm, 5.0 μm, pore size 100 Å, water-acetonitrile+0.1% TFA, gradient 5 to 87% for 10 min, retention time 6.31 min). MS (ESI) m/z 1045.3 [MH]⁺. ¹H NMR (400 MHz, DMSO-d₆). δ 9.32 (s, 1H), 9.00-8.92 (m, 2H), 8.64-8.50 (m, 2H), 8.17 (dd, J=5.3, 1.3 Hz, 1H), 7.47-7.32 (m, 5H), 7.24-7.06 (m, 4H), 6.96-6.86 (m, 3H), 6.75 (d, J=6.8 Hz, 1H), 6.46 (t, J=5.87 Hz, 1H), 5.15 (d, J=3.3 Hz, 1H), 5.13-5.04 (m, 1H), 4.61-4.50 (m, 3H), 4.50-4.31 (m, 3H), 4.30-4.20 (m, 1H), 4.07 (s, 3H), 3.96-3.82 (m, 4H), 3.70-3.56 (m, 2H), 3.52 (t, J=6.1 Hz, 2H), 3.43 (t, J=6.2 Hz, 2H), 3.38-3.33 (m, 2H), 2.44 (s, 3H), 2.11-2.01 (m, 1H), 1.95-1.83 (m, 1H), 1.82-1.71 (m, 2H), 1.71-1.61 (m, 2H), 1.58-1.46 (m, 2H), 1.46-1.33 (m, 5H), 0.93 (s, 9H).

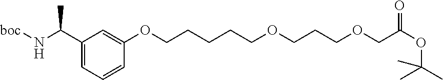

Compound 4. (S)-tert-butyl 2-(3-((5-(3-(1-((tert-butoxycarbonyl)amino)ethyl) phenoxy)pentyl)oxy)propoxy)acetate. Yellowish oil. Yield 99%. LCMS (C18 column 20×2 mm, 2.5 μm, pore size 100 Å, water-acetonitrile+0.1% TFA, gradient 5 to 87% for 3 min, retention time 2.13 min). MS (ESI) m/z 496.8 [MH]⁺.

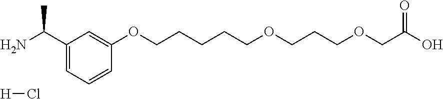 [SMILES: C[C@H](N)c1cccc(OCCCCCOCCCOCC(=O)O)c1 ; Cl]

Compound 5. (S)-2-(3-((5-(3-(1-aminoethyl)phenoxy)pentyl)oxy)propoxy)acetic acid hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.00 min). MS (ESI) m/z 340.5 [MH]⁺.

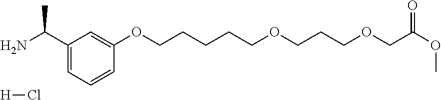 [SMILES: COC(=O)COCCCOCCCCCOc1cccc([C@H](C)N)c1 ; Cl]

Compound 6. (S)-methyl 2-(3-((5-(3-(1-aminoethyl)phenoxy)pentyl)oxy)propoxy)acetate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.14 min). MS (ESI) m/z 354.4 [MH]⁺.

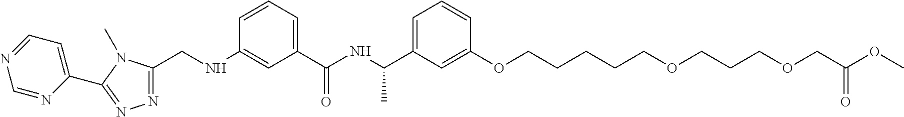 [SMILES: COC(=O)COCCCOCCCCCOc1cccc([C@H](C)NC(=O)c2cccc(NCc3nnc(-c4ccncn4)n3C)c2)c1]

Compound 8. (S)-methyl 2-(3-((5-(3-(1-(3-(((4-methyl-5-(pyrimidin-4-yl)-4H-1,2,4-triazol-3-yl)methyl)amino)benzamido)ethyl)phenoxy)pentyl)oxy)propoxy)acetate. Light brown solid. Yield 98%. LCMS (C18 column 20×2 mm, 2.5 μm, pore size 100 Å, water-acetonitrile+0.1% TFA, gradient 5 to 87% for 3 min, retention time 1.51 min). MS (ESI) m/z 646.5 [MH]⁺.

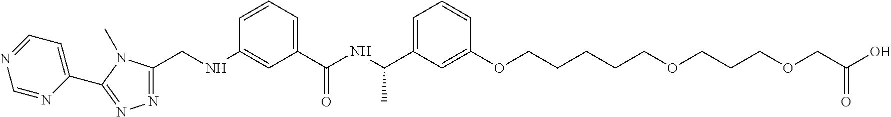 [SMILES: C[C@H](NC(=O)c1cccc(NCc2nnc(-c3ccncn3)n2C)c1)c1cccc(OCCCCCOCCCOCC(=O)O)c1]

Compound 9. (S)-2-(3-((5-(3-(1-(3-(((4-methyl-5-(pyrimidin-4-yl)-4H-1,2,4-triazol-3-yl)methyl)amino)benzamido)ethyl)phenoxy)pentyl)oxy)propoxy)acetic acid. Light brown solid. Yield 79%. LCMS (C18 column 20×2 mm, 2.5 μm, pore size 100 Å, water-acetonitrile+0.1% TFA, gradient 5 to 87% for 3 min, retention time 1.38 min). MS (ESI) m/z 632.8[MH]⁺.

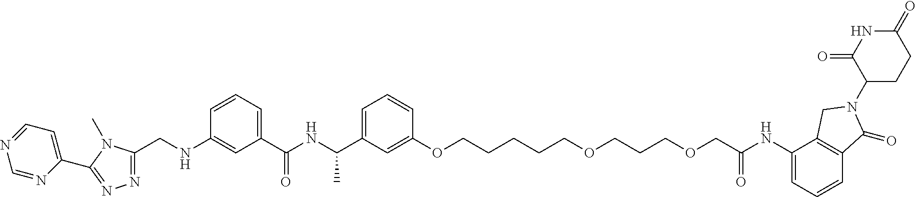 [SMILES: C[C@H](NC(=O)c1cccc(NCc2nnc(-c3ccncn3)n2C)c1)c1cccc(OCCCCCOCCCOCC(=O)Nc2cccc3c2CN(C2CCC(=O)NC2=O)C3=O)c1]

Compound 10. D185. N-((1S)-1-(3-((5-(3-(2-((2-(2,6-dioxopiperidin-3-yl)-1-oxoisoindolin-4-yl)amino)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 48%. LCMS (C18 column 100×4.6 mm, 5.0 μm, pore size 100 Å, water-acetonitrile+0.1% TFA, gradient 5 to 87% for 10 min, retention time 5.83 min). MS (ESI) m/z 873.4 [MH]⁺. ¹H NMR (400 MHz, DMSO-d₆). δ 11.00 (s, 1H), 9.69 (s, 1H), 9.33 (d, J=1.3 Hz, 1H), 8.96 (d, J=5.3 Hz, 1H), 8.56 (d, J=8.5 Hz, 1H), 8.17 (dd, J=5.3, 1.4 Hz, 1H), 7.73 (d, J=7.9 Hz, 1H), 7.59-7.46 (m, 2H), 7.25-7.13 (m, 3H), 7.10 (d, J=7.7 Hz, 1H), 6.97-6.86 (m, 3H), 6.75 (d, J=8.0 Hz, 1H), 6.46 (t, J=5.14 Hz, 1H), 5.19-5.01 (m, 2H), 4.57 (d, J=5.6 Hz, 2H), 4.37 (d, J=9.0 Hz, 2H), 4.10-4.03 (m, 5H), 3.91 (t, J=6.2 Hz, 2H), 3.58 (t, J=6.5 Hz, 2H), 3.45 (t, J=6.3 Hz, 2H), 3.38-3.34 (m, 2H), 2.97-2.83 (m, 1H), 2.69-2.55 (m, 1H), 2.42-2.28 (m, 1H), 2.07-1.94 (m, 1H), 1.87-1.74 (m, 2H), 1.73-1.61 (m, 2H), 1.57-1.34 (m, 7H).

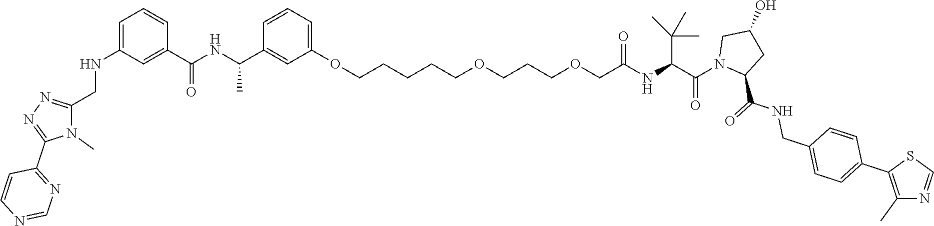 [SMILES: Cc1ncsc1-c1ccc(CNC(=O)[C@@H]2C[C@@H](O)CN2C(=O)[C@@H](NC(=O)COCCCOCCCCCOc2cccc([C@H](C)NC(=O)c3cccc(NCc4nnc(-c5ccncn5)n4C)c3)c2)C(C)(C)C)cc1]

Compound 10. D184. (2S,4R)-1-((S)-3,3-dimethyl-2-(2-(3-((5-(3-((S)-1-(3-(((4-methyl-5-(pyrimidin-4-yl)-4H-1,2,4-triazol-3-yl)methyl)amino)benzamido)ethyl)phenoxy)pentyl)oxy) propoxy)acetamido)butanoyl)-4-hydroxy-N-(4-(4-methylthiazol-5-yl)benzyl)pyrrolidine-2-carboxamide. Colorless solid. Yield 40%. LCMS (C18 column 100×4.6 mm, 5.0 μm, pore size 100 Å, water-acetonitrile+0.1% TFA, gradient 5 to 87% for 10 min, retention time 6.31 min). MS (ESI) m/z 1045.1 [MH]⁺. ¹H NMR (400 MHz, DMSO-d₆). δ 9.32 (s, 1H), 9.00-8.92 (m, 2H), 8.64-8.50 (m, 2H), 8.17 (dd, J=5.3, 1.3 Hz, 1H), 7.47-7.32 (m, 5H), 7.24-7.06 (m, 4H), 6.96-6.86 (m, 3H), 6.75 (d, J=6.8 Hz, 1H), 6.46 (t, J=5.87 Hz, 1H), 5.15 (d, J=3.3 Hz, 1H), 5.13-5.04 (m, 1H), 4.61-4.50 (m, 3H), 4.50-4.31 (m, 3H), 4.30-4.20 (m, 1H), 4.07 (s, 3H), 3.96-3.82 (m, 4H), 3.70-3.56 (m, 2H), 3.52 (t, J=6.1 Hz, 2H), 3.43 (t, J=6.2 Hz, 2H), 3.38-3.33 (m, 2H), 2.44 (s, 3H), 2.11-2.01 (m, 1H), 1.95-1.83 (m, 1H), 1.82-1.71 (m, 2H), 1.71-1.61 (m, 2H), 1.58-1.46 (m, 2H), 1.46-1.33 (m, 5H), 0.93 (s, 9H).

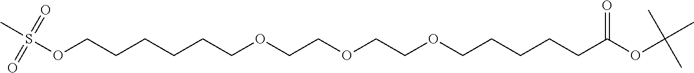 [SMILES: CC(C)(C)OC(=O)CCCCCOCCOCCOCCCCCCOS(C)(=O)=O]

Compound 2. Tert-butyl 3,10,13,16-tetraoxa-2-thiadocosan-22-oate 2,2-dioxide. Colorless oil. Yield 100%. The product was used in the next step immediately. ¹H NMR (400 MHz, DMSO-d₆) δ 4.18 (t, J=6.5 Hz, 2H), 3.54-3.42 (m, 8H), 3.37 (dd, J=11.6, 6.5 Hz, 4H), 3.16 (s, 3H), 2.17 (t, J=7.3 Hz, 2H), 1.64 (p, J=14.0, 6.7 Hz, 2H), 1.56-1.42 (m, 6H), 1.39 (s, 9H), 1.36-1.20 (m, 6H).

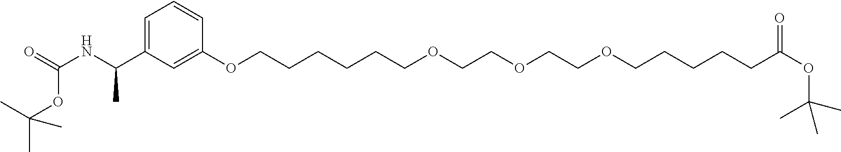 [SMILES: C[C@@H](NC(=O)OC(C)(C)C)c1cccc(OCCCCCCOCCOCCOCCCCCC(=O)OC(C)(C)C)c1]

Compound 4. (R)-tert-butyl 6-(2-(2-((6-(3-(1-((tert-butoxycarbonyl)amino)ethyl) phenoxy)hexyl)oxy)ethoxy)ethoxy)hexanoate. Yellowish oil. Yield 99%. LCMS (C18 column 20×2 mm, 2.5 μm, pore size 100 Å, water-acetonitrile+0.1% TFA, gradient 5 to 87% for 3 min, retention time 2.42 min). MS (ESI) m/z 596.7 [MH]⁺.

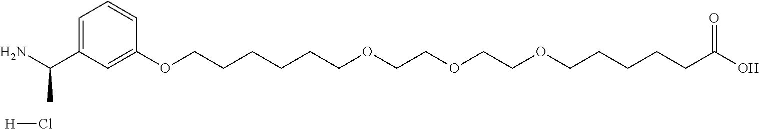

Compound 5. (R)-6-(2-(2-((6-(3-(1-aminoethyl)phenoxy)hexyl)oxy)ethoxy)ethoxy)hexanoic acid hydrochloride. Light brown solid. Yield 100%. LCMS (C18 column 20×2 mm, 2.5 μm, pore size 100 Å, water-acetonitrile+0.1% TFA, gradient 5 to 87% for 3 min, retention time 1.20 min). MS (ESI) m/z 440.5 [MH]⁺.

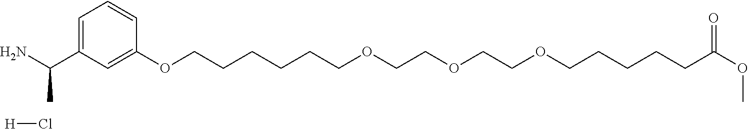

Compound 6. (R)-methyl 6-(2-(2-((6-(3-(1-aminoethyl)phenoxy)hexyl)oxy)ethoxy)ethoxy)hexanoate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.30 min). MS (ESI) m/z 454.5 [MH]⁺.

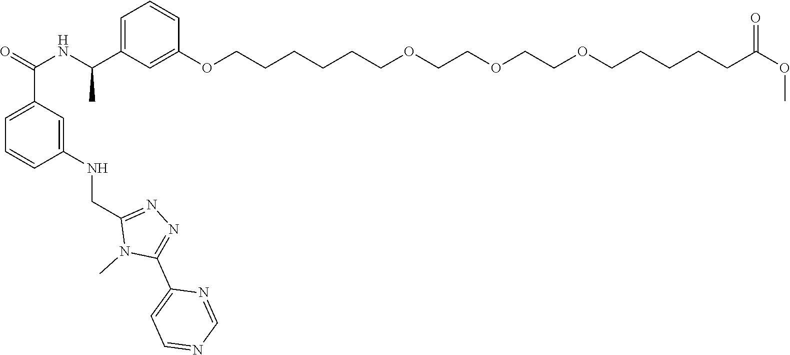 [SMILES: COC(=O)CCCCCOCCOCCOCCCCCCOc1cccc([C@@H](C)NC(=O)c2cccc(NCc3nnc(-c4ccncn4)n3C)c2)c1]

Compound 8. (R)-methyl 6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoate.

Yellowish solid. Yield 97%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 746.7 [MH]⁺.

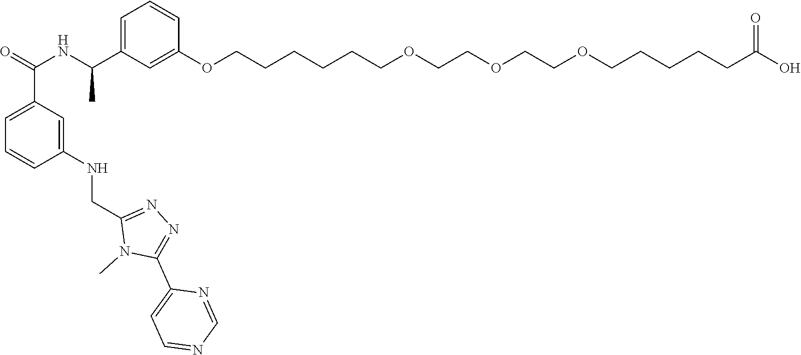 [SMILES: C[C@@H](NC(=O)c1cccc(NCc2nnc(-c3ccncn3)n2C)c1)c1cccc(OCCCCCCOCCOCCOCCCCCC(=O)O)c1]

Compound 9 (R)-6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoic acid.

Yellowish solid. Yield 82%. LCMS (C18 column 20×2 mm, 2.5 μm, pore size 100 Å, water-acetonitrile+0.1% TFA, gradient 5 to 87% for 3 min, retention time 1.46 min). MS (ESI) m/z 732.7 [MH]⁺.

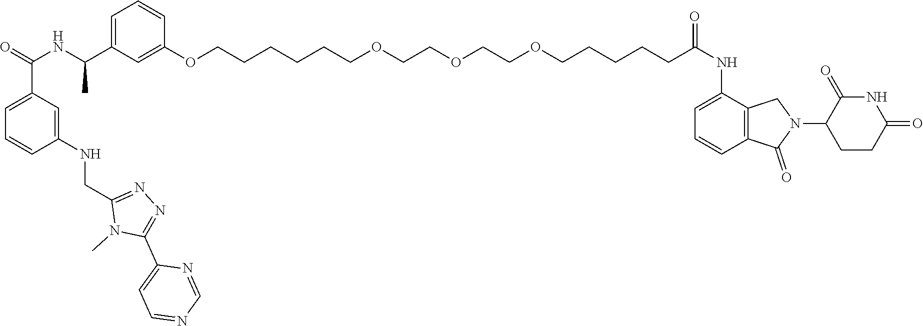 [SMILES: C[C@@H](NC(=O)c1cccc(NCc2nnc(-c3ccncn3)n2C)c1)c1cccc(OCCCCCCOCCOCCOCCCCCC(=O)Nc2cccc3c2CN(C2CCC(=O)NC2=O)C3=O)c1]

Compound 10. D57. N-((1R)-1-(3-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Colorless solid. 20%. LCMS (C18 column 100×4.6 mm, 5.0 μm, pore size 100 Å, water-acetonitrile+0.1% TFA, gradient 5 to 87% for 10 min, retention time 6.18 min). MS (ESI) m/z 975.2 [MH]⁺. ¹H NMR (400 MHz, DMSO-d₆) δ 11.00 (s, 1H), 9.74 (s, 1H), 9.33 (s, 1H), 8.97 (d, J=5.3 Hz, 1H), 8.55 (d, J=8.4 Hz, 1H), 8.17 (d, J=5.2 Hz, 1H), 7.81 (d, J=7.0 Hz, 1H), 7.58-7.39 (m, 2H), 7.26-7.07 (m, 4H), 6.99-6.85 (m, 3H), 6.76 (d, J=6.8 Hz, 1H), 6.45 (t, J=5.38 Hz, 1H), 5.21-4.98 (m, 2H), 4.57 (d, J=5.3 Hz, 2H), 4.43-4.23 (m, 2H), 4.07 (s, 3H), 3.92 (t, J=6.5 Hz, 2H), 3.52-3.41 (m, 8H), 3.36 (dd, J=15.1, 6.6 Hz, 4H), 2.98-2.82 (m, 1H), 2.68-2.55 (m, 1H), 2.39-2.21 (m, 3H), 2.07-1.92 (m, 1H), 1.74-1.18 (m, 17H).

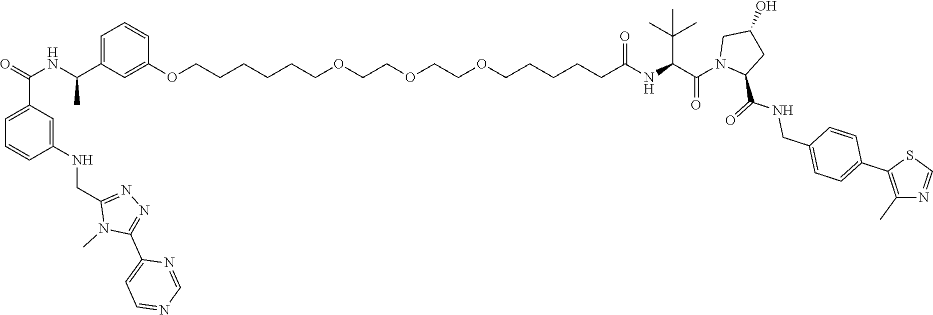 [SMILES: Cc1ncsc1-c1ccc(CNC(=O)[C@@H]2C[C@@H](O)CN2C(=O)[C@@H](NC(=O)CCCCCOCCOCCOCCCCCCOc2cccc([C@@H](C)NC(=O)c3cccc(NCc4nnc(-c5ccncn5)n4C)c3)c2)C(C)(C)C)cc1]

Compound 10. D79. (2S,4R)-1-((S)-2-(tert-butyl)-22-(3-((R)-1-(3-(((4-methyl-5-(pyrimidin-4-yl)-4H-1,2,4-triazol-3-yl)methyl)amino)benzamido)ethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless solid. 35%. LCMS (C18 column 100×4.6 mm, 5.0 μm, pore size 100 Å, water-acetonitrile+0.1% TFA, gradient 5 to 87% for 10 min, retention time 6.61 min). MS (ESI) m/z 1144.8 [MH]⁺. ¹H NMR (400 MHz, DMSO-d₆) δ 9.33 (s, 1H), 9.02-8.90 (m, 2H), 8.59-8.48 (m, 2H), 8.17 (d, J=5.2 Hz, 1H), 7.82 (d, J=9.1 Hz, 1H), 7.40 (q, J=8.2 Hz, 4H), 7.23-7.03 (m, 4H), 6.97-6.85 (m, 3H), 6.76 (d, J=7.1 Hz, 1H), 6.45 (t, J=5.50, 1H), 5.15-5.02 (m, 2H), 4.61-4.50 (m, J=13.1, 7.7 Hz, 3H), 4.48-4.38 (m, 2H), 4.38-4.30 (m, 1H), 4.26-4.16 (m, 1H), 4.07 (s, 3H), 3.92 (t, J=6.4 Hz, 2H), 3.74-3.59 (m, 2H), 3.52-3.40 (m, 8H), 3.39-3.32 (m, 4H), 2.44 (s, 3H), 2.29-2.20 (m, 1H), 2.16-1.97 (m, 2H), 1.94-1.84 (m, 1H), 1.75-1.63 (m, 2H), 1.58-1.18 (m, 16H), 0.93 (s, 9H).

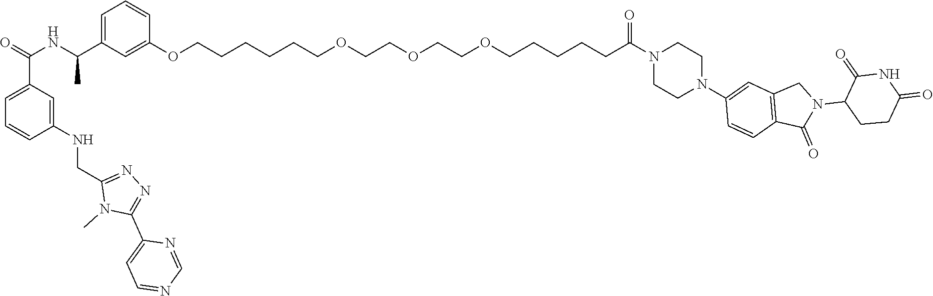 [SMILES: C[C@@H](NC(=O)c1cccc(NCc2nnc(-c3ccncn3)n2C)c1)c1cccc(OCCCCCCOCCOCCOCCCCCC(=O)N2CCN(c3ccc4c(c3)CN(C3CCC(=O)NC3=O)C4=O)CC2)c1]

Compound 10. D191. N-((1R)-1-(3-((6-(2-(2-((6-(4-(2-(2,6-dioxopiperidin-3-yl)-1-oxoisoindolin-5-yl)piperazin-1-yl)-6-oxohexyl)oxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Colorless solid. 35%. LCMS (C18 column 100×4.6 mm, 5.0 μm, pore size 100 Å, water-acetonitrile+0.1% TFA, gradient 5 to 87% for 10 min, retention time 5.96 min). MS (ESI) m/z 1043.4 [MH]⁺. ¹H NMR (400 MHz, DMSO-d₆) δ 10.94 (s, 1H), 9.33 (d, J=1.3 Hz, 1H), 8.96 (d, J=5.3 Hz, 1H), 8.55 (d, J=8.2 Hz, 1H), 8.17 (d, J=5.3 Hz, 1H), 7.53 (d, J=8.4 Hz, 1H), 7.18 (m, 3H), 7.07 (m, 3H), 6.92 (m, 3H), 6.76 (d, J=7.9 Hz, 1H), 6.46 (t, J=5.7 Hz, 1H), 5.06 (m, 2H), 4.57 (d, J=5.7 Hz, 2H), 4.32 (d, J=16.9 Hz, 1H), 4.21 (d, J=16.9 Hz, 1H), 4.06 (s, 3H), 3.92 (t, J=6.5 Hz, 2H), 3.58 (m, 4H), 3.47 (m, 8H), 3.36 (t, J=6.5 Hz, 4H), 3.26 (m, 4H), 2.90 (m, 1H), 2.61 (m, 1H), 2.33 (m, 3H), 1.95 (m, 1H), 1.68 (m, 2H), 1.50 (m, 6H), 1.43 (d, J=7.1 Hz, 3H), 1.34 (m, 6H).

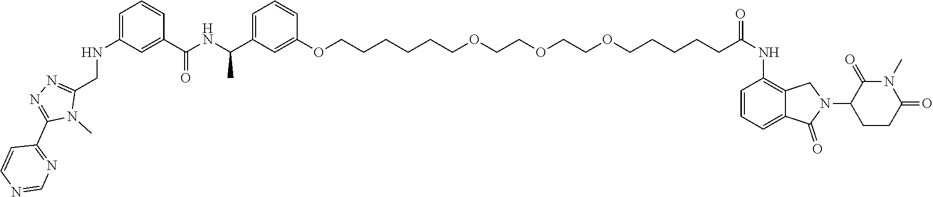 [SMILES: C[C@@H](NC(=O)c1cccc(NCc2nnc(-c3ccncn3)n2C)c1)c1cccc(OCCCCCCOCCOCCOCCCCCC(=O)Nc2cccc3c2CN(C2CCC(=O)N(C)C2=O)C3=O)c1]

Compound 10. D190. N-((1R)-1-(3-((6-(2-(2-((6-(4-(2-(1-methyl-2,6-dioxopiperidin-3-yl)-1-oxoisoindolin-5-yl)piperazin-1-yl)-6-oxohexyl)oxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Colorless solid. 25%. LCMS (C18 column 100×4.6 mm, 5.0 μm, pore size 100 Å, water-acetonitrile+0.1% TFA, gradient 5 to 87% for 10 min, retention time 6.34 min). MS (ESI) m/z 988.2 [MH]⁺. ¹H NMR (400 MHz, DMSO-d₆) δ 9.76 (s, 1H), 9.33 (d, J=1.4 Hz, 1H), 8.97 (d, J=5.2 Hz, 1H), 8.55 (d, J=8.2 Hz, 1H), 8.17 (dd, J=5.3, 1.4 Hz, 1H), 7.80 (m, 1H), 7.49 (m, 2H), 7.18 (m, 3H), 7.10 (d, J=7.9 Hz, 1H), 6.91 (m, 3H), 6.75 (m, 1H), 6.46 (t, J=5.7 Hz, 1H), 5.21 (dd, J=13.4, 5.1 Hz, 1H), 5.09 (p, J=7.0 Hz, 1H), 4.57 (d, J=5.7 Hz, 2H), 4.33 (m, 2H), 4.07 (s, 3H), 3.92 (t, J=6.5 Hz, 2H), 3.46 (m, 8H), 3.36 (m, 4H), 3.01 (s, 3H), 2.94 (m, 1H), 2.76 (m, 1H), 2.32 (m, 3H), 2.03 (m, 1H), 1.65 (m, 4H), 1.49 (m, 4H), 1.43 (d, J=7.1 Hz, 3H), 1.35 (m, 6H).

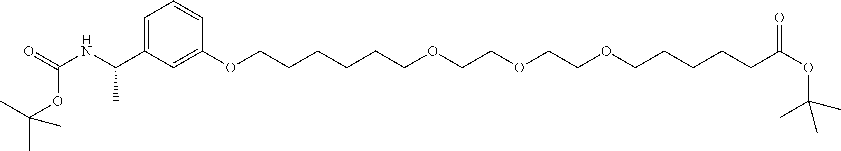 [SMILES: C[C@H](NC(=O)OC(C)(C)C)c1cccc(OCCCCCCOCCOCCOCCCCCC(=O)OC(C)(C)C)c1]

Compound 4. (S)-tert-butyl 6-(2-(2-((6-(3-(1-((tert-butoxycarbonyl)amino)ethyl)phenoxy) hexyl)oxy)ethoxy)ethoxy)hexanoate. Yellowish oil. Yield 95%. LCMS (C18 column 20×2 mm, 2.5 μm, pore size 100 Å, water-acetonitrile+0.1% TFA, gradient 5 to 87% for 3 min, retention time 2.50 min). MS (ESI) m/z 596.8 [MH]⁺.

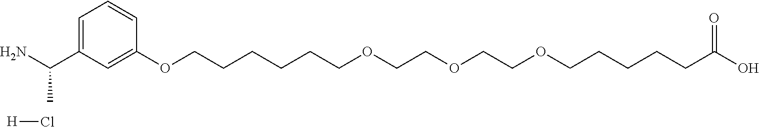

Compound 5 (S)-6-(2-(2-((6-(3-(1-aminoethyl)phenoxy)hexyl)oxy)ethoxy)ethoxy)hexanoic acid hydrochloride. Light brown solid. Yield 100%. LCMS (C18 column 20×2 mm, 2.5 μm, pore size 100 Å, water-acetonitrile+0.1% TFA, gradient 5 to 87% for 3 min, retention time 1.17 min). MS (ESI) m/z 440.5 [MH]⁺.

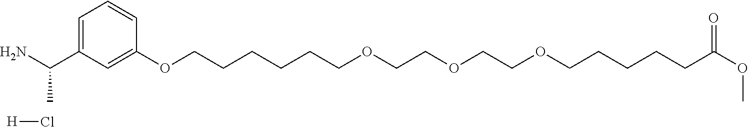

Compound 6. (S)-methyl 6-(2-(2-((6-(3-(1-aminoethyl)phenoxy)hexyl)oxy)ethoxy)ethoxy)hexanoate hydrochloride. Light brown solid. Yield 96%. LCMS (C18 column 20×2 mm, 2.5 μm, pore size 100 Å, water-acetonitrile+0.1% TFA, gradient 5 to 87% for 3 min, retention time 1.31 min). MS (ESI) m/z 454.5 [MH]⁺.

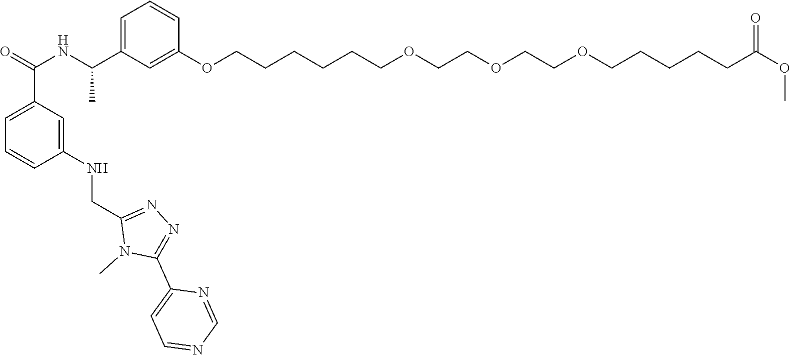 [SMILES: COC(=O)CCCCCOCCOCCOCCCCCCOc1cccc([C@H](C)NC(=O)c2cccc(NCc3nnc(-c4ccncn4)n3C)c2)c1]

Compound 8. (S)-methyl 6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoate. Yellowish solid. Yield 98%. LCMS (C18 column 20×2 mm, 2.5 μm, pore size 100 Å, water-acetonitrile+0.1% TFA, gradient 5 to 87% for 3 min, retention time 1.65 min). MS (ESI) m/z 746.8 [MH]⁺.

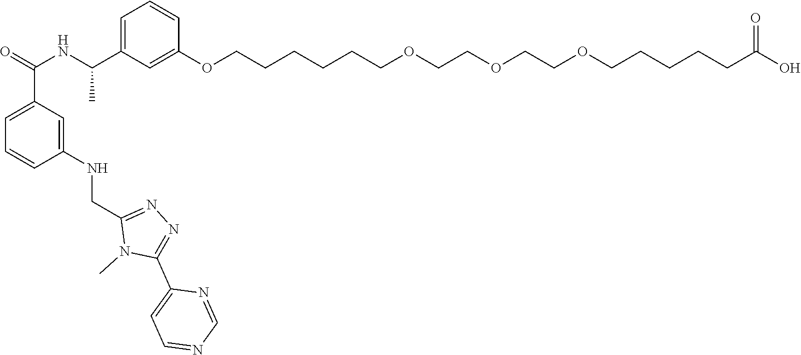 [SMILES: C[C@H](NC(=O)c1cccc(NCc2nnc(-c3ccncn3)n2C)c1)c1cccc(OCCCCCCOCCOCCOCCCCCC(=O)O)c1]

Compound 9. (S)-6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoic acid. Yellowish solid. Yield 71%. LCMS (C18 column 20×2 mm, 2.5 μm, pore size 100 Å, water-acetonitrile+0.1% TFA, gradient 5 to 87% for 3 min, retention time 1.45 min). MS (ESI) m/z 732.7 [MH]⁺.

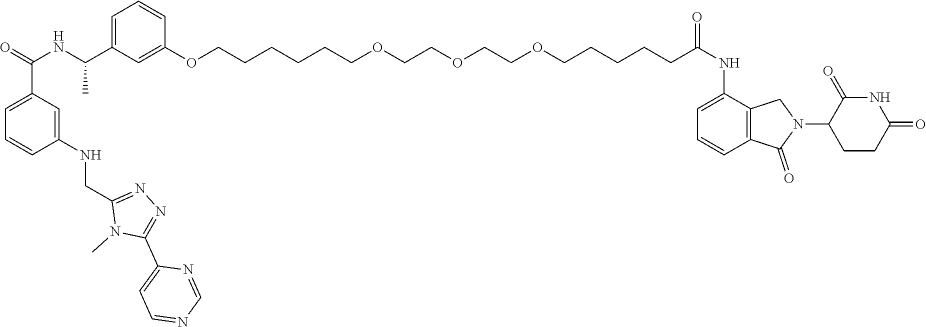 [SMILES: C[C@H](NC(=O)c1cccc(NCc2nnc(-c3ccncn3)n2C)c1)c1cccc(OCCCCCCOCCOCCOCCCCCC(=O)Nc2cccc3c2CN(C2CCC(=O)NC2=O)C3=O)c1]

Compound 10. D55. N-((1S)-1-(3-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Colorless solid. 16%. LCMS (C18 column 100×4.6 mm, 5.0 μm, pore size 100 Λ, water-acetonitrile+0.1% TFA, gradient 5 to 87% for 10 min, retention time 6.23 min). MS (ESI) m/z 974.3 [MH]⁺. ¹H NMR (400 MHz, DMSO-d₆) 11.01 (s, 1H), 9.74 (s, 1H), 9.33 (d, J=1.3 Hz, 1H), 8.97 (d, J=5.3 Hz, 1H), 8.55 (d, J=8.4 Hz, 1H), 8.17 (dd, J=5.2, 1.3 Hz, 1H), 7.81 (d, J=7.3 Hz, 1H), 7.58-7.38 (m, 2H), 7.27-7.06 (m, 4H), 6.99-6.86 (m, 3H), 6.82-6.71 (m, 1H), 6.45 (t, 1H), 5.21-5.02 (m, 2H), 4.57 (d, J=5.3 Hz, 2H), 4.35 (q, J=17.9 Hz, 2H), 4.07 (s, 3H), 3.92 (t, J=6.4 Hz, 2H), 3.51-3.41 (m, 8H), 3.36 (dd, J=15.3, 6.5 Hz, 4H), 2.98-2.85 (m, 1H), 2.68-2.56 (m, 1H), 2.39-2.29 (m, 3H), 2.06-1.95 (m, 1H), 1.73-1.23 (m, 17H).

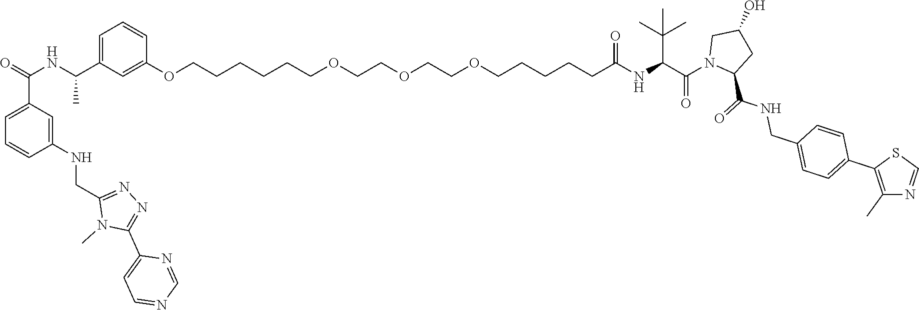 [SMILES: Cc1ncsc1-c1ccc(CNC(=O)[C@@H]2C[C@@H](O)CN2C(=O)[C@@H](NC(=O)CCCCCOCCOCCOCCCCCCOc2cccc([C@H](C)NC(=O)c3cccc(NCc4nnc(-c5ccncn5)n4C)c3)c2)C(C)(C)C)cc1]

Compound 10. D78. (2S,4R)-1-((S)-2-(tert-butyl)-22-(3-((S)-1-(3-(((4-methyl-5-(pyrimidin-4-yl)-4H-1,2,4-triazol-3-yl)methyl)amino)benzamido)ethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless solid. 32%. LCMS (C18 column 100×4.6 mm, 5.0 μm, pore size 100 Å, water-acetonitrile+0.1% TFA, gradient 5 to 87% for 10 min, retention time 6.61 min). MS (ESI) m/z 1144.8 [MH]⁺. ¹H NMR (400 MHz, DMSO-d₆) 9.33 (d, J=1.3 Hz, 1H), 9.03-8.91 (m, 2H), 8.59-8.48 (m, 2H), 8.17 (d, J=5.3 Hz, 1H), 7.83 (d, J=9.3 Hz, 1H), 7.39 (q, J=8.4 Hz, 4H), 7.25-7.06 (m, 4H), 6.97-6.85 (m, 3H), 6.75 (d, J=9.0 Hz, 1H), 6.46 (t, J=5.50 Hz, 1H), 5.16-5.01 (m, 1H), 4.62-4.48 (m, 3H), 4.48-4.37 (m, 2H), 4.37-4.29 (m, 1H), 4.28-4.14 (m, 1H), 4.07 (s, 3H), 3.92 (t, J=6.4 Hz, 2H), 3.72-3.59 (m, 2H), 3.54-3.39 (m, 8H), 3.39-3.31 (m, 4H), 2.44 (s, 3H), 2.23 (s, 1H), 2.16-1.98 (m, 2H), 1.96-1.83 (m, 1H), 1.75-1.61 (m, 2H), 1.55-1.18 (m, 16H), 0.93 (s, 9H).

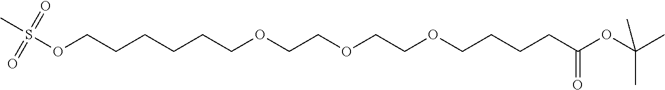 [SMILES: CC(C)(C)OC(=O)CCCCOCCOCCOCCCCCCOS(C)(=O)=O]

Compound 2. Tert-butyl 3,10,13,16-tetraoxa-2-thiahenicosan-21-oate 2,2-dioxide. Colorless oil. Yield 92%. The product was used in the next step immediately.

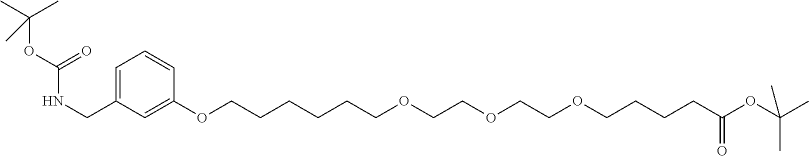 [SMILES: CC(C)(C)OC(=O)CCCCOCCOCCOCCCCCCOc1cccc(CNC(=O)OC(C)(C)C)c1]

Compound 4. tert-butyl 5-(2-(2-((6-(3-(((tert-butoxycarbonyl)amino)methyl)phenoxy)hexyl) oxy)ethoxy)ethoxy)pentanoate. Yellowish oil. Yield 100%. LCMS (C18 column 20×2 mm, 2.5 μm, pore size 100 Å, water-acetonitrile+0.1% TFA, gradient 5 to 87% for 3 min, retention time 2.30 min). MS (ESI) m/z 568.8 [MH]⁺.

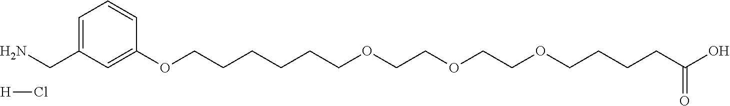

Compound 5. 5-(2-(2-((6-(3-(aminomethyl)phenoxy)hexyl)oxy)ethoxy)ethoxy)pentanoic acid hydrochloride. Light brown solid. Yield 100%. LCMS (C18 column 20×2 mm, 2.5 μm, pore size 100 Å, water-acetonitrile+0.1% TFA, gradient 5 to 87% for 3 min, retention time 1.10 min). MS (ESI) m/z 412.5 [MH]⁺.

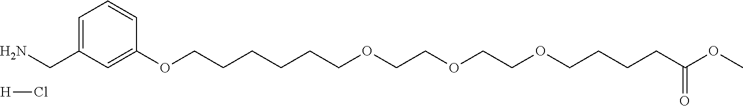

Compound 6. Methyl 5-(2-(2-((6-(3-(aminomethyl)phenoxy)hexyl)oxy)ethoxy)ethoxy)pentanoate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.24 min). MS (ESI) m/z 426.4 [MH]⁺.

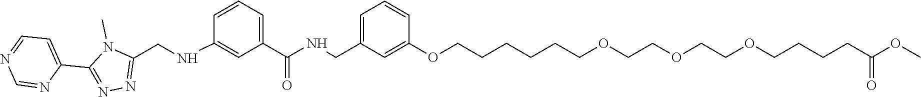 [SMILES: COC(=O)CCCCOCCOCCOCCCCCCOc1cccc(CNC(=O)c2cccc(NCc3nnc(-c4ccncn4)n3C)c2)c1]

Compound 8. Methyl 5-(2-(2-((6-(3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)pentanoate. Light brown solid. Yield 98%. LCMS (C18 column 20×2 mm, 2.5 μm, pore size 100 Å, water-acetonitrile+0.1% TFA, gradient 5 to 87% for 3 min, retention time 1.55 min). MS (ESI) m/z 718.7 [MH]⁺.

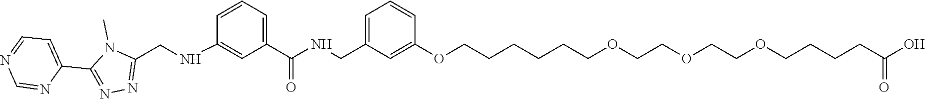 [SMILES: Cn1c(CNc2cccc(C(=O)NCc3cccc(OCCCCCCOCCOCCOCCCCC(=O)O)c3)c2)nnc1-c1ccncn1]

Compound 9. 5-(2-(2-((6-(3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)pentanoic acid. Light brown solid. Quantitative yield. LCMS (C18 column 20×2 mm, 2.5 μm, pore size 100 Å, water-acetonitrile+0.1% TFA, gradient 5 to 87% for 3 min, retention time 1.39 min). MS (ESI) m/z 705.0 [MH]⁺.

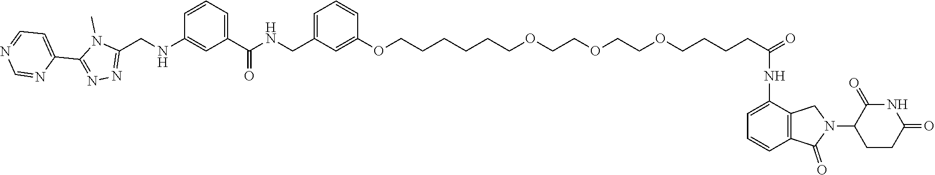 [SMILES: Cn1c(CNc2cccc(C(=O)NCc3cccc(OCCCCCCOCCOCCOCCCCC(=O)Nc4cccc5c4CN(C4CCC(=O)NC4=O)C5=O)c3)c2)nnc1-c1ccncn1]

Compound 10. D108. N-(3-((6-(2-(2-((5-((2-(2,6-dioxopiperidin-3-yl)-1-oxoisoindolin-4-yl)amino)-5-oxopentyl)oxy)ethoxy)ethoxy)hexyl)oxy)benzyl)-3-(((4-methyl-5-(pyrimidin-4-yl)-4H-1,2,4-triazol-3-yl)methyl)amino)benzamide. Colorless solid. LCMS (C18 column 100×4.6 mm, 5.0 μm, pore size 100 Å, water-acetonitrile+0.1% TFA, gradient 5 to 87% for 10 min, retention time 5.93 min). MS (ESI) m/z 945.8 [MH]⁺. ¹H NMR (400 MHz, DMSO-d₆) δ 11.01 (s, 1H), 9.75 (s, 1H), 9.33 (s, 1H), 8.97 (d, J=5.2 Hz, 1H), 8.83-8.75 (m, 1H), 8.17 (d, J=4.0 Hz, 1H), 7.81 (d, J=6.8 Hz, 1H), 7.48 (d, J=7.1 Hz, 2H), 7.29-7.06 (m, 4H), 6.96-6.74 (m, 4H), 6.50-6.45 (m, 1H), 5.13 (s, 1H), 4.57 (d, J=5.3 Hz, 2H), 4.40 (d, J=5.2 Hz, 2H), 4.35 (d, J=8.4 Hz, 2H), 4.07 (s, 3H), 3.90 (t, J=6.4 Hz, 2H), 3.57-3.33 (m, 8H), 3.01-2.83 (m, 1H), 2.71-2.56 (m, 2H), 2.41-2.29 (m, 4H), 2.08-1.95 (m, 1H), 1.79-1.21 (m, 14H).

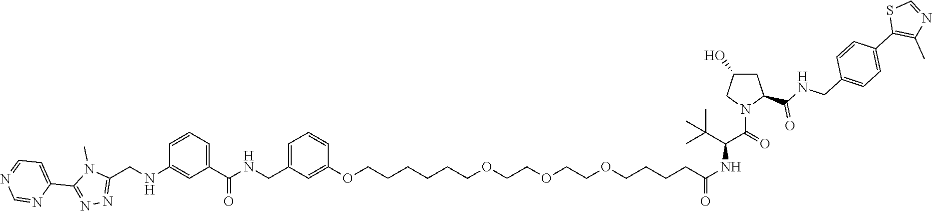 [SMILES: Cc1ncsc1-c1ccc(CNC(=O)[C@@H]2C[C@@H](O)CN2C(=O)[C@@H](NC(=O)CCCCOCCOCCOCCCCCCOc2cccc(CNC(=O)c3cccc(NCc4nnc(-c5ccncn5)n4C)c3)c2)C(C)(C)C)cc1]

Compound 10. D109. (2S,4R)-1-((S)-2-(tert-butyl)-21-(3-((3-(((4-methyl-5-(pyrimidin-4-yl)-4H-1,2,4-triazol-3-yl)methyl)amino)benzamido)methyl)phenoxy)-4-oxo-9,12,15-trioxa-3-azahenicosan-1-oyl)-4-hydroxy-N-(4-(4-methylthiazol-5-yl)benzyl)pyrrolidine-2-carboxamide. Colorless solid. LCMS (C18 column 100×4.6 mm, 5.0 μm, pore size 100 Å, water-acetonitrile+0.1% TFA, gradient 5 to 87% for 10 min, retention time 6.30 min). MS (ESI) m/z 1117.0 [MH]⁺. ¹H NMR (400 MHz, DMSO-d₆) δ 9.36-9.30 (m, 1H), 9.01-8.94 (m, 2H), 8.81 (t, J=5.5 Hz, 1H), 8.55 (t, J=5.5 Hz, 1H), 8.17 (dd, J=5.3, 1.4 Hz, 1H), 7.84 (d, J=9.3 Hz, 1H), 7.39 (q, J=8.2 Hz, 4H), 7.27-7.06 (m, 4H), 6.90 (d, J=7.8 Hz, 1H), 6.88-6.82 (m, 2H), 6.78 (d, J=8.7 Hz, 1H), 6.47 (t, J=5.5 Hz, 1H), 5.11 (d, J=3.6 Hz, 1H), 4.64-4.51 (m, 3H), 4.48-4.38 (m, 4H), 4.34 (s, 1H), 4.25-4.16 (m, 1H), 4.07 (s, 3H), 3.91 (t, J=6.5 Hz, 2H), 3.72-3.58 (m, 2H), 3.47 (dd, J=15.5, 5.2 Hz, 8H), 3.36 (t, J=6.4 Hz, 4H), 2.44 (s, 3H), 2.31-2.20 (m, 1H), 2.18-1.97 (m, 2H), 1.95-1.84 (m, 1H), 1.73-1.62 (m, 2H), 1.58-1.26 (m, 10H), 0.93 (s, 9H).

### Example 7: Preparation of Intermediate 3

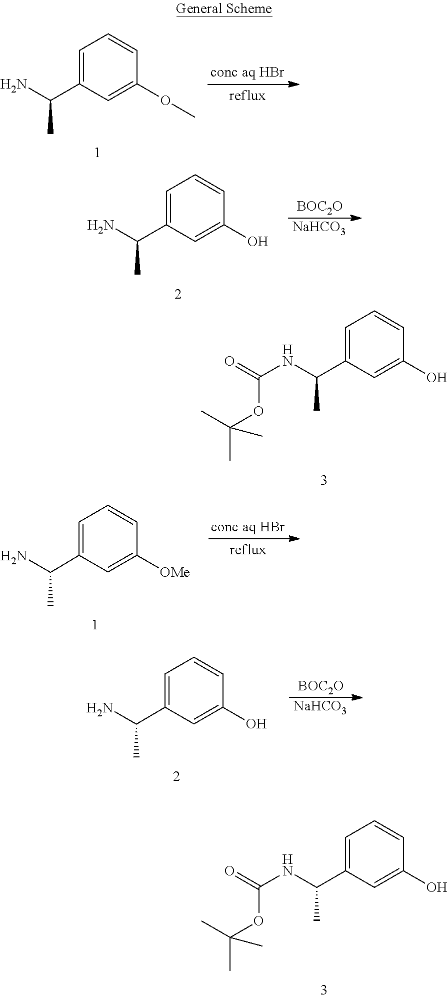

### Example 8: Preparation of Compounds D76 and D52

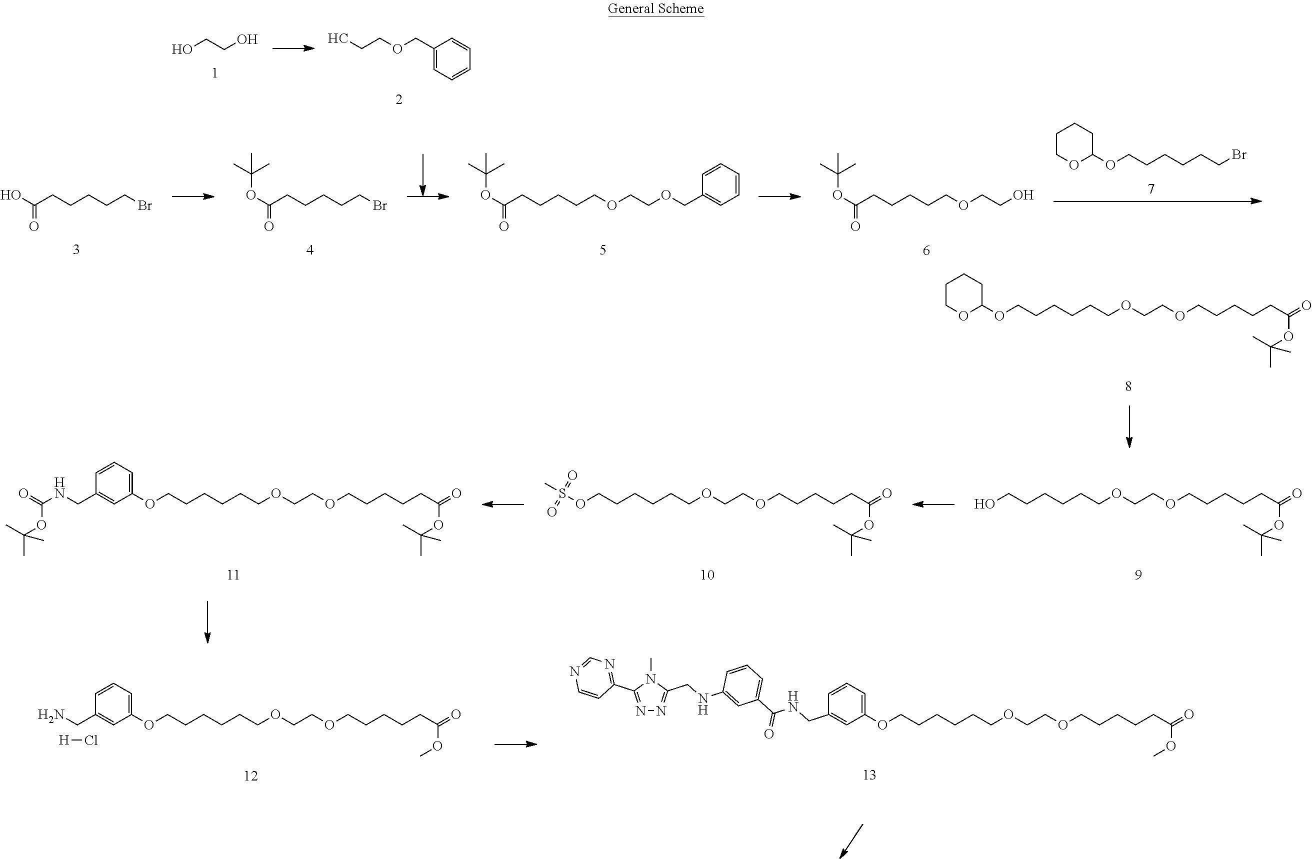

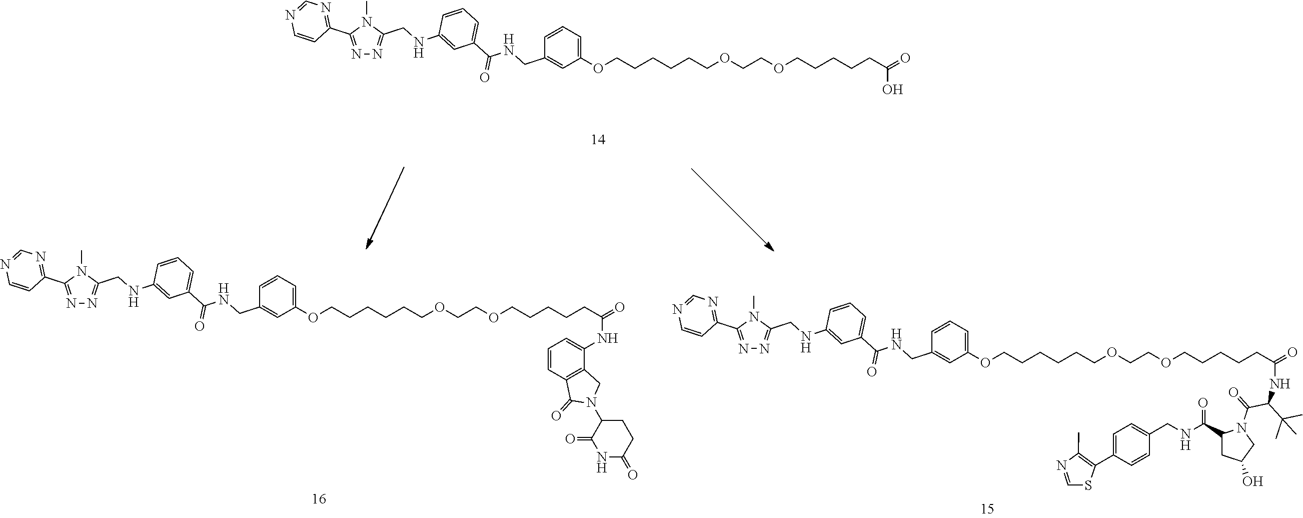

### Compounds, Procedures, and Analytical Data

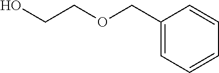 [SMILES: OCCOCc1ccccc1]

A mixture of glycol 1 (40 mL, 44.5 g, 0.717 mol), benzyl chloride (20 mL, 22 g, 0.173 mol) and aqueous 50% sodium hydroxide (50 mL) is heated with stirring at 100° C. for 24 h. The mixture is cooled, diluted with water (100 mL) and extracted with ether (3×40 mL). The organic extracts are combined, dried with magnesium sulfate, filtered and concentrated under vacuum. The product 2 was purified by chromatography on silica, using chloroform as an eluent. Yield 9 g (34%). ¹H NMR (400 MHz, CDCl₃) δ 7.43-7.29 (m, 5H), 4.58 (s, 2H), 3.78 (s, 2H), 3.69-3.51 (m, 2H), 2.10 (s, 1H).

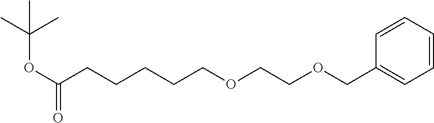 [SMILES: CC(C)(C)OC(=O)CCCCCOCCOCc1ccccc1]

To a solution of alcohol 2 (7 g, 46 mmol) in toluene (240 mL), tert-butyl 6-bromohexanoate (22 g, 87 mmol), tetrabutylammonium bromide (1.4 g, 4.3 mmol) and a solution of NaOH (42 g, 1.05 mol) in water (120 mL) were added. The reaction mixture was stirred at reflux for 48 hrs. The mixture is cooled, diluted with water (200 mL) and extracted with ether (3×100 mL). The organic extracts are combined, dried with magnesium sulfate, filtered and concentrated under vacuum. The product 5 was purified by chromatography on silica, using chloroform as an eluent. Yield 7.1 g (47%). ¹H NMR (400 MHz, CDCl₃) δ 7.40-7.28 (m, 5H), 4.59 (s, 2H), 3.70-3.54 (m, 4H), 3.48 (t, J=7.5 Hz, 2H), 2.22 (t, J=7.5 Hz, 2H), 1.69-1.54 (m, 4H), 1.45 (s, 9H), 1.43-1.33 (m, 2H).

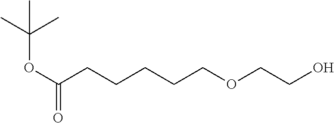 [SMILES: CC(C)(C)OC(=O)CCCCCOCCO]

To a solution of ester 5 (7.1 g, 22.02 mmol) in methanol (50 mL), Pd on carbon (10%, 0.7 g) was carefully added. The reaction mixture was flushed with hydrogen and stirred at ambient temperature in hydrogen atmosphere for overnight. Then the mixture was filtered through celite and concentration the filtrate to leave pure product 6 5.1 g (100%). ¹H NMR (400 MHz, CDCl₃) δ 3.74 (t, J=6.5 Hz, 2H), 3.54 (t, J=6.5 Hz, 2H), 3.49 (t, J=6.5 Hz, 2H), 2.23 (t, J=7.4 Hz, 2H), 1.90 (s, 1H), 1.62 (tt, J=13.8, 7.2 Hz, 4H), 1.45 (s, 9H), 1.43-1.36 (m, 2H).

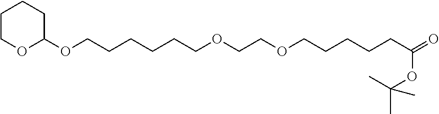 [SMILES: CC(C)(C)OC(=O)CCCCCOCCOCCCCCCOC1CCCCO1]

To a solution of alcohol 6 (0.75 g, 3.2 mmol) in toluene (17 mL), 2-[(6-bromohexyl)oxy]tetrahydro-2H-pyran (1.04 g, 3.92 mmol), tetrabutylammonium bromide (0.1 g, 0.31 mmol) and a solution of NaOH (3 g, 75 mmol) in water (8.5 mL) were added. The reaction mixture was stirred at reflux for 48 hrs. The mixture is cooled, diluted with water (100 mL) and extracted with ether (3×10 mL). The organic extracts are combined, dried with magnesium sulfate, filtered and concentrated under vacuum. The product 8 was purified by chromatography on silica, using chloroform as an eluent. Yield 1.06 g (78%). ¹H NMR (400 MHz, CDCl₃) δ 4.58-4.57 (m, 1H), 3.94-3.82 (m, 2H), 3.78-3.72 (m, 2H), 3.57-3.37 (m, 6H), 2.27-2.18 (m, 2H), 1.94-1.78 (m, 2H), 1.75-1.68 (m, 2H), 1.67-1.47 (m, 14H), 1.45 (s, 9H), 1.44-1.33 (m, 4H).

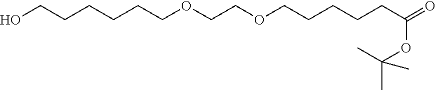 [SMILES: CC(C)(C)OC(=O)CCCCCOCCOCCCCCCO]

To a solution of ester 8 (1.06 g, 2.5 mmol) in methanol (5 mL), toluene sulphonic acid hydrate (48 mg, 0.25 mmol) was added and the reaction mixture was stirred at ambient temperature for 2 hours. Then the solvent was stripped off and the residue was partitioned between methylene chloride (10 mL) and water (10 mL). Organic phase was separated and aqueous phase was extracted with methylene chloride (10 mL). Combined organic phases were dried over MgSO₄ and concentrated. Yield of 9 was 0.75 g, (88%). ¹H NMR (400 MHz, CDCl₃) δ 3.73 (d, J=4.0 Hz, 1H), 3.68-3.65 (m, 1H), 3.56 (d, J=8.8 Hz, 2H), 3.53 (d, J=4.4 Hz, 1H), 3.49 (t, J=6.5 Hz, 2H), 2.22 (td, J=7.4, 4.7 Hz, 2H), 2.10 (s, 1H), 1.66-1.58 (m, 10H), 1.45 (s, 12H), 1.43-1.34 (m, 4H).

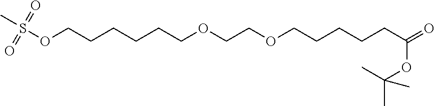 [SMILES: CC(C)(C)OC(=O)CCCCCOCCOCCCCCCOS(C)(=O)=O]

Yield 0.42 g, (100%).

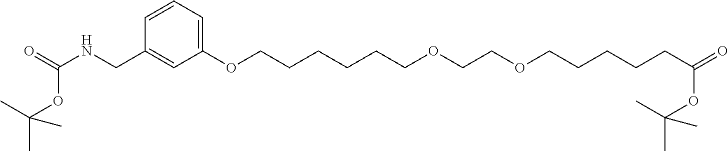 [SMILES: CC(C)(C)OC(=O)CCCCCOCCOCCCCCCOc1cccc(CNC(=O)OC(C)(C)C)c1]

Colorless oil 96%. LCMS (C18 column 20×2 mm, 2.5 μm, pore size 100 Å, water-acetonitrile+0.1% TFA, gradient 5 to 87% for 3 min, retention time 2.53 min). MS (ESI) m/z 538.5 [MH]+.

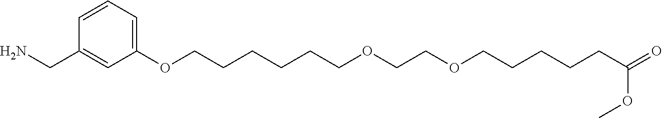 [SMILES: COC(=O)CCCCCOCCOCCCCCCOc1cccc(CN)c1]

Colorless oil 100%. LCMS (C18 column 20×2 mm, 2.5 μm, pore size 100 Å, water-acetonitrile+0.1% TFA, gradient 5 to 87% for 3 min, retention time 1.28 min). MS (ESI) m/z 396.5 [MH]+.

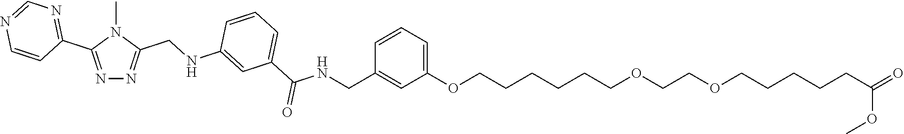 [SMILES: COC(=O)CCCCCOCCOCCCCCCOc1cccc(CNC(=O)c2cccc(NCc3nnc(-c4ccncn4)n3C)c2)c1]

Colorless oil 94%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 588.8 [MH]+.

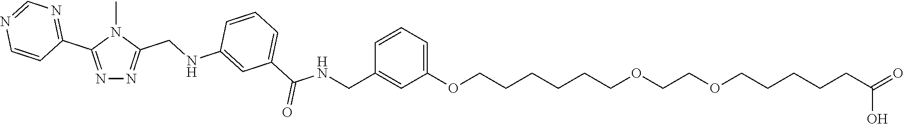 [SMILES: Cn1c(CNc2cccc(C(=O)NCc3cccc(OCCCCCCOCCOCCCCCC(=O)O)c3)c2)nnc1-c1ccncn1]

Colorless oil 95%. LCMS (C18 column 20×2 mm, 2.5 μm, pore size 100 Å, water-acetonitrile+0.1% TFA, gradient 5 to 87% for 3 min, retention time 1.46 min). MS (ESI) m/z 675.0 [MH]+.

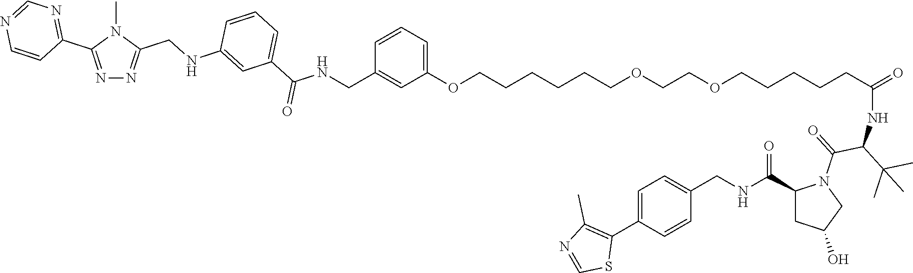 [SMILES: Cc1ncsc1-c1ccc(CNC(=O)[C@@H]2C[C@@H](O)CN2C(=O)[C@@H](NC(=O)CCCCCOCCOCCCCCCOc2cccc(CNC(=O)c3cccc(NCc4nnc(-c5ccncn5)n4C)c3)c2)C(C)(C)C)cc1]

D76. To a solution of acid 14 (0.18 g, 0.267 mmol) in dried pyridine (2 mL), VHL hydrochloride (0.14 g, 0.299 mmol) was added, followed by TBTU (0.13 g, 0.342 mmol). The reaction mixture was stirred at ambient temperature and evaporated dryness. The residue was partitioned between methylene chloride (5 mL) and water (5 mL). The organic layer was separated; aqueous one was extracted with methylene chloride (5 mL). Combined organic layers were dried over MgSO₄ and concentrated. The product was purified by HPLC chromatography. Colorless oil 36%. LCMS (C18 column 20×2 mm, 2.5 μm, pore size 100 Å, water-acetonitrile+0.1% TFA, gradient 5 to 87% for 10 min, retention time 6.58 min). MS (ESI) m/z 1087.8 [MH]+. ¹H NMR (400 MHz, DMSO-d₆) δ 9.33 (s, 1H), 8.97 (s, 1H), 8.96 (d, J=5.5 Hz, 1H), 8.81 (t, J=6.6 Hz, 1H), 8.55 (t, J=6.6 Hz, 1H), 8.17 (d, J=6.6 Hz, 1H), 7.83 (d, J=9.3 Hz, 1H), 7.39 (q, J=8.1 Hz, 4H), 7.24 (s, 1H), 7.19 (dd, J=16.8, 8.0 Hz, 2H), 7.10 (d, J=8.2 Hz, 1H), 6.90 (d, J=9.1 Hz, 1H), 6.85 (d, J=6.7 Hz, 1H), 6.84 (s, 1H), 6.78 (d, J=9.0 Hz, 1H), 6.47 (t, J=6.6 Hz, 1H), 5.11 (d, J=3.5 Hz, 1H), 4.57 (d, J=6.5 Hz, 2H), 4.53 (d, J=9.0 Hz, 2H), 4.46-4.37 (m, 4H), 4.34 (s, 1H), 4.24-4.16 (m, 1H), 4.07 (s, 3H), 3.91 (t, J=6.5 Hz, 2H), 3.67-3.61 (m, 2H), 3.44 (s, 3H), 3.38-3.31 (m, 2H), 2.44 (s, 3H), 2.33-2.20 (m, 1H), 2.13-1.97 (m, 2H), 1.93-1.83 (m, 1H), 1.70-1.62 (m, 2H), 1.55-1.42 (m, 7H), 1.40-1.18 (m, 7H), 0.96-0.90 (s, 9H).

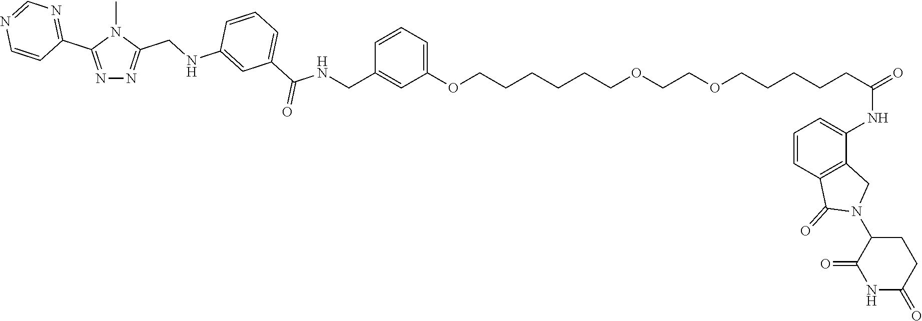 [SMILES: Cn1c(CNc2cccc(C(=O)NCc3cccc(OCCCCCCOCCOCCCCCC(=O)Nc4cccc5c4CN(C4CCC(=O)NC4=O)C5=O)c3)c2)nnc1-c1ccncn1]

D52. To a solution of acid 14 (0.18 g, 0.267 mmol) in dried pyridine (2 mL), aniline (0.10 g, 0.385 mmol) was added, followed by TBTU (0.2 g, 0.527 mmol). The reaction mixture was stirred at ambient temperature and evaporated dryness. The residue was partitioned between methylene chloride (5 mL) and water (5 mL). The organic layer was separated; aqueous one was extracted with methylene chloride (5 mL). Combined organic layers were dried over MgSO₄ and concentrated. The product was purified by HPLC chromatography. Colorless oil. LCMS (C18 column 20×2 mm, 2.5 μm, pore size 100 Å, water-acetonitrile+0.1% TFA, gradient 5 to 87% for 10 min, retention time 6.11 min). MS (ESI) m/z 915.8 [MH]+. ¹H NMR (400 MHz, DMSO-d₆) δ 11.01 (s, 1H), 9.75 (s, 1H), 9.33 (s, 1H), 8.97 (d, J=5.3 Hz, 1H), 8.81 (t, J=5.3 Hz, 1H), 8.17 (d, J=5.3, 1H), 7.81 (d, J=7.0 Hz, 1H), 7.54-7.41 (m, 2H), 7.24 (s, 1H), 7.19 (dd, J=16.4, 8.1 Hz, 2H), 7.10 (d, J=7.8 Hz, 1H), 6.90 (d, J=7.5 Hz, 1H), 6.85 (d, J=8.5 Hz, 2H), 6.77 (d, J=9.5 Hz, 1H), 6.47 (t, J=5.3 Hz, 1H), 5.14 (dd, J=13.1, 5.1 Hz, 1H), 4.57 (d, J=5.5 Hz, 2H), 4.40 (d, J=5.6 Hz, 2H), 4.35 (d, J=7.4 Hz, 1H), 4.07-4.03 (m, 4H), 3.91 (t, J=6.4 Hz, 1H), 3.44 (s, 3H), 3.43-3.33 (m, 2H), 2.96-2.85 (m, 1H), 2.68-2.57 (m, 1H), 2.35 (t, J=7.7 Hz, 2H), 2.27 (t, J=7.4 Hz, 1H), 2.06-1.96 (m, 1H), 1.71-1.64 (m, 2H), 1.63-1.56 (m, 2H), 1.54-1.43 (m, 6H), 1.40-1.23 (m, 8H).

### Example 9: Preparation of Compounds D91, D67, D88, D66, D54, D75, D53, D74, D92, D156, D188, and D187

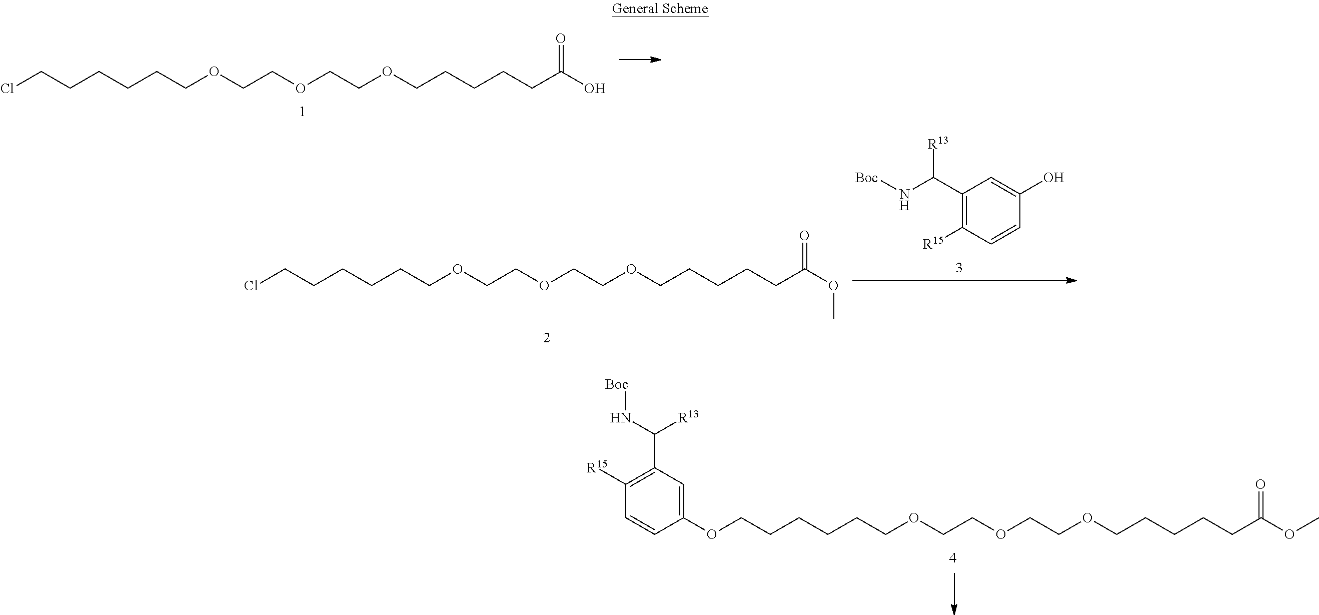

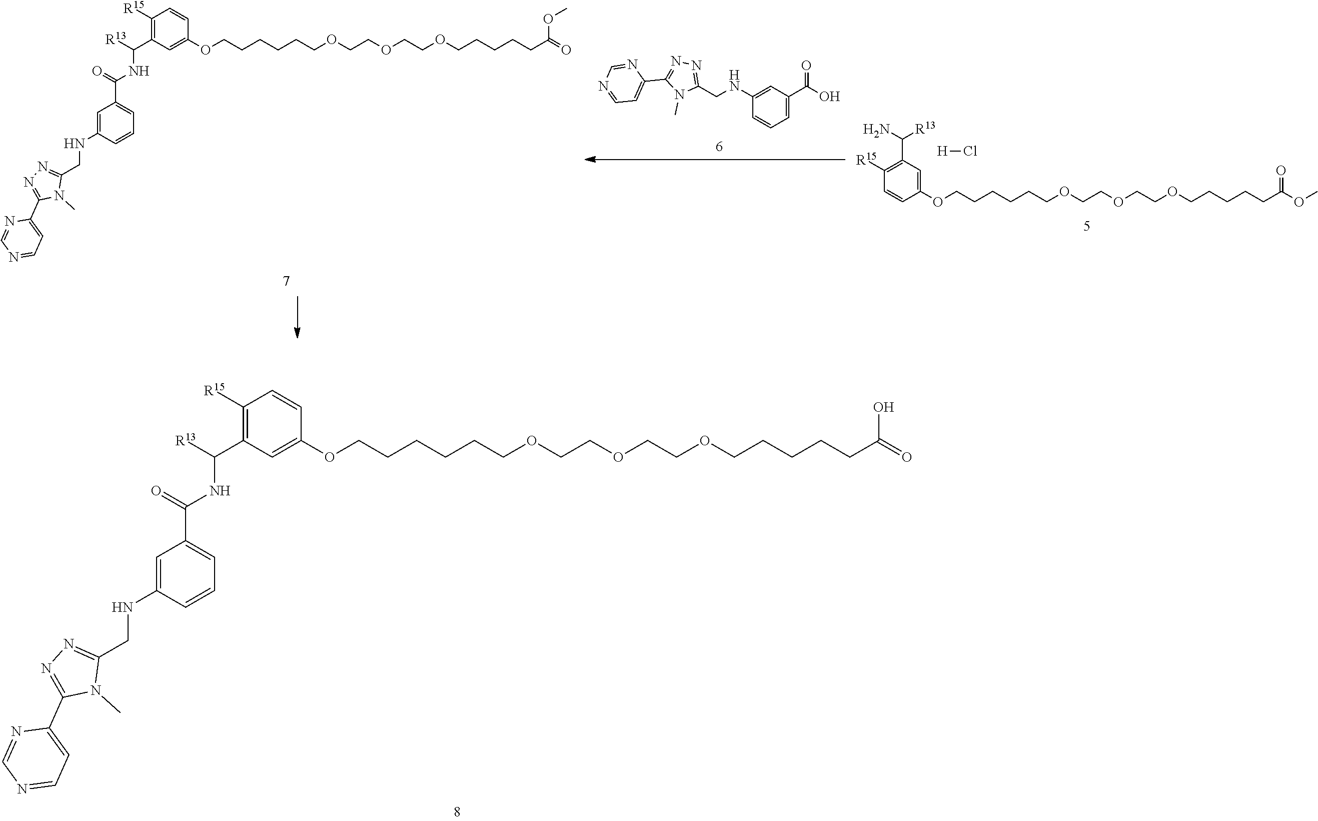

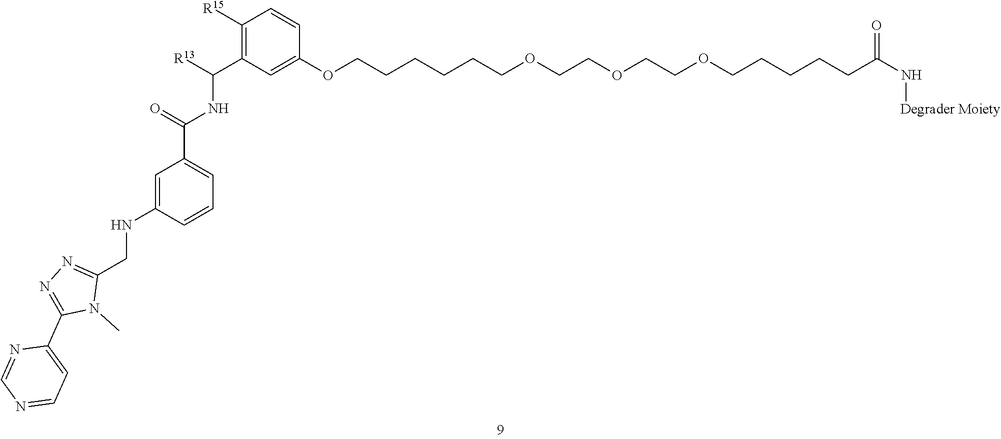

### General Procedures

Compound 2. Methyl 6-(2-(2-((6-chlorohexyl)oxy)ethoxy)ethoxy)hexanoate. To a solution of compound 1 (3.3 mmol, 1 eq) in dry MeOH (15 mL) was added dropwise SOCl₂ (0.48 mL, 6.6 mmol, 2 eq) at 0° C. The mixture was stirred for 12 h at r.t. and then evaporated to dryness to give the crude product 2 which no need additional purification. Colorless oil, yield 98%. ¹H NMR (400 MHz, DMSO-d₆) δ 3.61 (t, J=6.5 Hz, 2H), 3.57 (s, 3H), 3.53-3.47 (m, 4H), 3.45 (d, J=3.5 Hz, 4H), 3.36 (dd, J=9.7, 6.2 Hz, 4H), 2.28 (t, J=7.3 Hz, 2H), 1.69 (dd, J=13.9, 6.8 Hz, 2H), 1.59-1.43 (m, 6H), 1.41-1.19 (m, 6H).

Compound 4. Compound 3 (2.5 mmol, 1 eq), compound 2 (2.75 mmol, 1.1 eq), K₂CO₃ (5.0 mmol, 2 eq), KI (0.25 mmol, 0.1 eq) in dry acetonitrile (50 mL) were stirred for 12 h at reflux. The precipitate was filtered off, washed with acetonitrile. The solvent was evaporated to dryness. The residue was purified by silica gel flash column chromatography using chloroform and methanol as an eluent to afford the desired product 4.

Compound 5. A solution of compound 4 (2.5 mmol, 1 eq) in dry DCM (50 mL) with 12.5 mL 3M HCl in dioxane (5 eq) were stirred for 24 h at r.t. The mixture was evaporated to dryness to give the crude product 5 which was used in the next step without additional purification.

Compound 7. Compound 6 (1.2 mmol, 1 eq), compound 5 (1.2 mmol, 1 eq), hydroxybenzotriazole (HOBt) (1.2 mmol, 1 eq), 1-Ethyl-3-(3-dimethylaminopropyl) carbodiimide (EDCI) (1.32 mmol, 1.1 eq) and DIPEA (4.8 mmol, 4 eq) in dry dichloromethane (DCM) (30 mL) were stirred for 12 h at r.t. The reaction mixture was washed with water (3*10 mL), brine (2×10 mL), dried on activated Na₂SO₄, and concentrated in a rotary evaporator under reduced pressure at ambient temperature. The residue was purified by silica gel flash column chromatography using chloroform and methanol as an eluent to afford the desired product 7.

Compound 8. To a solution of compound 7 (1.2 mmol, 1 eq) in MeOH (20 mL) was added a solution KOH (3.6 mmol, 3 eq) in 3 mL H₂O. The mixture was stirred for 12-36 h at r.t. and then MeOH evaporated. To the residue was added 15 mL of H₂O and the mixture was acidified to pH 5 with 1N aqueous HCl and aqueous phase was extracted with 4*15 mL of DCM. Combined organic phases were washed with brine, dried over Na₂SO₄, and evaporated. The product 8 was used in the next step without additional purification.

Compound 9. Compound 8 (0.1 mmol, 1 eq), recruiter (0.1 mmol, 1 eq), 2-(1H-Benzotriazole-1-yl)-1,1,3,3-tetramethylaminium tetrafluoroborate (TBTU) (0.2 mmol, 2 eq) and N,N-Diisopropylethylamine (DIPEA) (0.4 mmol, 4 eq) in dry pyridine (3 mL) were stirred for 12 h at r.t. The mixture was evaporated to dryness. The residue was purified by HPLC in neutral conditions (YMC-Pack ODS-AQ 250×20 mml, S-10 μm, 12 nm, gradient water-acetonitrile).

### Compounds and Analytical Data

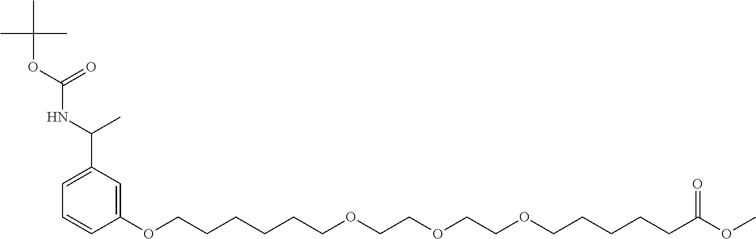 [SMILES: COC(=O)CCCCCOCCOCCOCCCCCCOc1cccc(C(C)NC(=O)OC(C)(C)C)c1]

Compound 4. Methyl 6-(2-(2-((6-(3-(1-((tert-butoxycarbonyl)amino)ethyl)phenoxy) hexyl)oxy)ethoxy)ethoxy)hexanoate. Colorless oil 94%. LCMS (C18 column 20×2 mm, 2.5 μm, pore size 100 Å, water-acetonitrile+0.1% TFA, gradient 5 to 87% for 3 min, retention time 2.04 min). MS (ESI) m/z 554.8 [MH]+. ¹H NMR (400 MHz, CDCl₃) δ 7.24 (t, J=7.2 Hz, 1H), 6.87 (d, J=7.2 Hz, 1H), 6.83 (s, 1H), 6.77 (d, J=7.2 Hz, 1H), 3.95 (t, J=7.9 Hz, 1H), 3.67-3.64 (m, 8H), 3.61-3.57 (m, 4H), 3.49-3.44 (m, 4H), 2.32 (t, J=7.9 Hz, 2H), 1.78 (t, J=7.9 Hz, 2H), 1.67-1.57 (m, 7H), 1.51-1.36 (m, 20H).

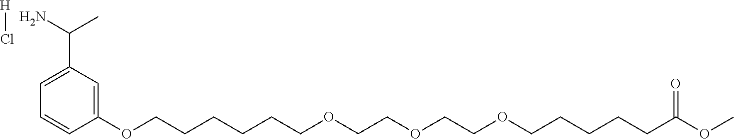

Compound 5. methyl 6-(2-(2-((6-(3-(1-aminoethyl)phenoxy)hexyl)oxy)ethoxy)ethoxy)hexanoate hydrochloride. Colorless oil 100%. LCMS (C18 column 20×2 mm, 2.5 μm, pore size 100 Å, water-acetonitrile+0.1% TFA, gradient 5 to 87% for 3 min, retention time 1.31 min). MS (ESI) m/z 454.6 [MH]+.

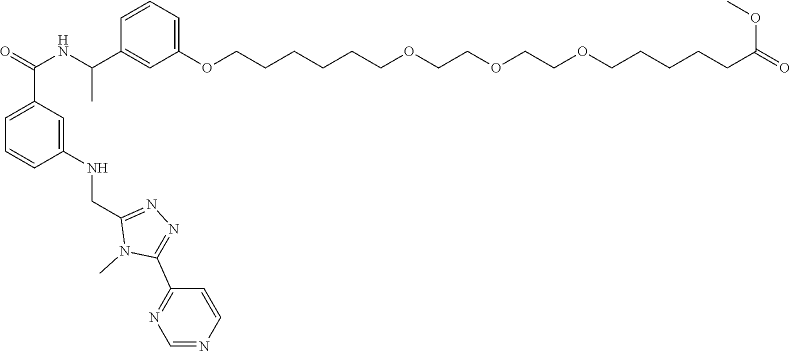 [SMILES: COC(=O)CCCCCOCCOCCOCCCCCCOc1cccc(C(C)NC(=O)c2cccc(NCc3nnc(-c4ccncn4)n3C)c2)c1]

Compound 7. methyl 6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoate. Colorless oil 75%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 476.8 [MH]+. ¹H NMR (400 MHz, CDCl₃) δ 9.28 (s, 1H), 8.87 (d, J=7.1 Hz, 1H), 8.27 (d, J=7.1 Hz, 1H), 7.27-7.22 (m, 2H), 7.08 (d, J=7.2 Hz, 1H), 6.93-6.90 (m, 2H), 6.80 (d, J=7.2 Hz, 1H), 6.46 (d, J=7.2 Hz, 1H), 5.30 (s, 2H), 5.30-5.28 (m, 1H), 4.95 (s, 2H), 4.17 (s, 2H), 3.95 (t, J=7.9 Hz, 2H), 3.67-3.64 (m, 9H), 3.63-3.56 (m, 11H), 3.50-3.43 (m, 5H), 2.31 (t, J=7.9 Hz, 2H), 1.79 (t, J=7.9 Hz, 2H), 1.68-1.57 (m, 11H), 1.51-1.36 (m, 8H).

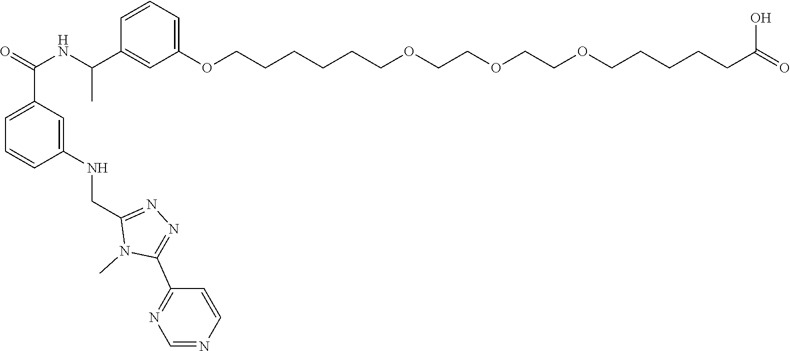 [SMILES: CC(NC(=O)c1cccc(NCc2nnc(-c3ccncn3)n2C)c1)c1cccc(OCCCCCCOCCOCCOCCCCCC(=O)O)c1]

Compound 8. 6-(2-(2-((6-(3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoic acid. Colorless oil 99%. LCMS (C18 column 20×2 mm, 2.5 μm, pore size 100 Å, water-acetonitrile+0.1% TFA, gradient 5 to 87% for 3 min, retention time 1.50 min). MS (ESI) m/z 732.8 [MH]+.

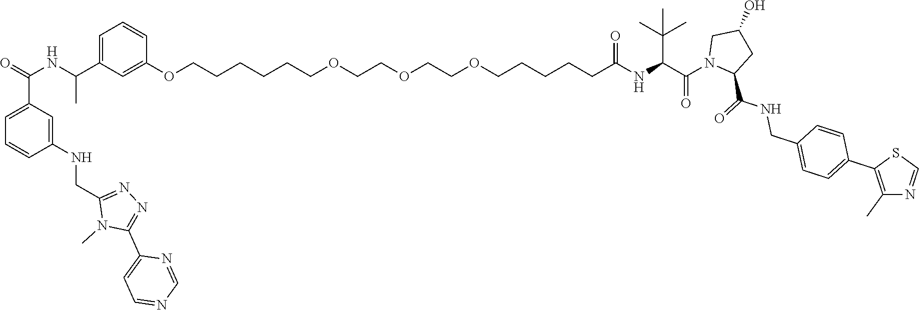 [SMILES: Cc1ncsc1-c1ccc(CNC(=O)[C@@H]2C[C@@H](O)CN2C(=O)[C@@H](NC(=O)CCCCCOCCOCCOCCCCCCOc2cccc(C(C)NC(=O)c3cccc(NCc4nnc(-c5ccncn5)n4C)c3)c2)C(C)(C)C)cc1]

Compound 9. D91. (2S,4R)-1-((2S)-2-(tert-butyl)-22-(3-(1-(3-(((4-methyl-5-(pyrimidin-4-yl)-4H-1,2,4-triazol-3-yl)methyl)amino)benzamido)ethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless oil 19%. LCMS (C18 column 100×4.6 mm, 5.0 μm, pore size 100 Å, water-acetonitrile+0.1% TFA, gradient 5 to 87% for 10 min, retention time 6.59 min). MS (ESI) m/z 1146.0 [MH]+. ¹H NMR (400 MHz, DMSO-d₆) δ 9.32 (s, 1H), 8.98 (s, 2H), 8.54 (t, J=6.1 Hz, 2H), 8.18 (d, J=6.1 Hz, 1H), 7.82 (d, J=7.1 Hz, 1H), 7.42-7.38 (m, 5H), 7.22-7.16 (m, 3H), 7.10 (d, J=7.2 Hz, 1H), 6.92-6.90 (m, 3H), 6.76 (d, J=7.2 Hz, 1H), 6.44 (t, J=7.2 Hz, 1H), 5.11-5.07 (m, 2H), 4.59-4.52 (m, 3H), 4.46-4.40 (m, 2H), 4.36-4.32 (m, 1H), 4.23-4.18 (m, 1H), 4.06 (s, 3H), 3.92 (t, J=7.9 Hz, 2H), 3.66-3.64 (m, 2H), 3.50-3.42 (m, 11H), 3.37-3.30 (m, 57H), 2.43 (s, 3H), 2.29-2.21 (m, 1H), 2.14-2.00 (m, 3H), 1.93-1.86 (m, 1H), 1.71-1.65 (m, 2H), 1.53-1.22 (m, 19H), 0.92 (s, 11H).

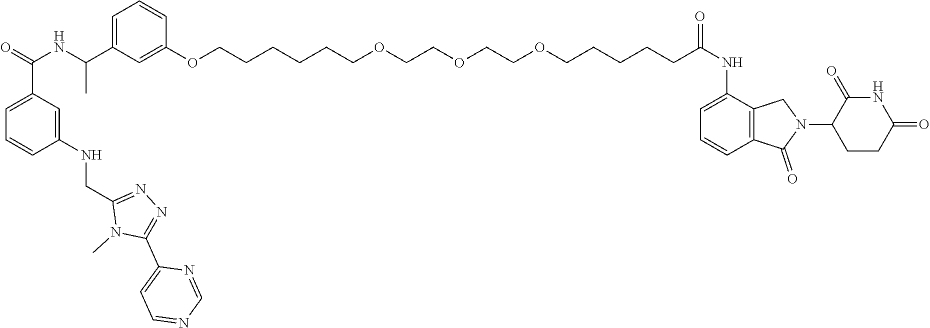 [SMILES: CC(NC(=O)c1cccc(NCc2nnc(-c3ccncn3)n2C)c1)c1cccc(OCCCCCCOCCOCCOCCCCCC(=O)Nc2cccc3c2CN(C2CCC(=O)NC2=O)C3=O)c1]

Compound 9. D67. N-(1-(3-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Colorless oil 19%. LCMS (C18 column 100×4.6 mm, 5.0 μm, pore size 100 Å, water-acetonitrile+0.1% TFA, gradient 5 to 87% for 10 min, retention time 6.18 min). MS (ESI) m/z 974.6 [MH]+. ¹H NMR (400 MHz, DMSO-d₆) δ 11.00 (s, 1H), 9.74 (s, 1H), 9.32 (s, 1H), 8.97 (d, J=6.1 Hz, 1H), 8.56 (d, J=7.1 Hz, 1H), 8.26 (d, J=6.1 Hz, 1H), 7.81 (d, J=7.1 Hz, 1H), 7.50-7.46 (m, 2H), 7.20-7.16 (m, 3H), 7.08 (d, J=7.2 Hz, 1H), 6.93-6.88 (m, 3H), 6.76 (d, J=7.2 Hz, 1H), 6.44 (t, J=7.2 Hz, 1H), 5.12 (tt, J=7.2 Hz, J=2.2 Hz, 2H), 4.56 (d, J=6.2 Hz, 2H), 4.35 (dd, J=12.2 Hz, J=7.2 Hz, 2H), 4.06 (s, 3H), 3.91 (t, J=7.9 Hz, 2H), 3.49-3.43 (m, 8H), 3.37-3.31 (m, 11H), 2.90 (m, 1H), 2.60 (m, 1H), 2.34 (t, J=6.2 Hz, 3H), 2.2 (m, 1H), 1.70-1.58 (m, 4H), 1.52-1.48 (m, 4H), 1.43 (d, J=7.2 Hz, 3H), 1.38-1.30 (m, 5H).

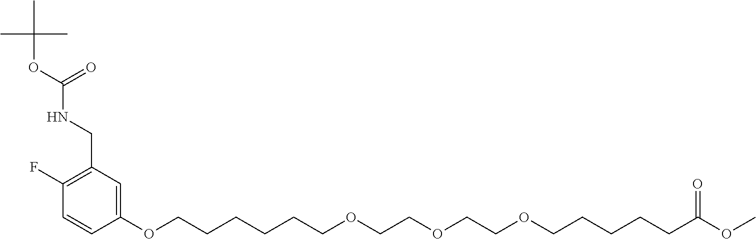 [SMILES: COC(=O)CCCCCOCCOCCOCCCCCCOc1ccc(F)c(CNC(=O)OC(C)(C)C)c1]

Compound 4. methyl 6-(2-(2-((6-(3-(((tert-butoxycarbonyl)amino)methyl)-4-fluorophenoxy)hexyl)oxy)ethoxy)ethoxy)hexanoate. Colorless oil 94%. LCMS (C18 column 20×2 mm, 2.5 μm, pore size 100 Å, water-acetonitrile+0.1% TFA, gradient 5 to 87% for 3 min, retention time 2.01 min). MS (ESI) m/z 558.6 [MH]+. ¹H NMR (400 MHz, CDCl₃) δ 6.93 (t, J=7.2 Hz, 1H), 6.86-6.83 (m, 1H), 6.83 (s, 1H), 6.75-6.71 (m, 1H), 4.95-4.90 (m, 1H), 4.33 (s, 2H), 3.90 (t, J=7.9 Hz, 2H), 3.67-3.64 (m, 8H), 3.60-3.58 (m, 4H), 3.49-3.44 (m, 4H), 2.31 (t, J=7.9 Hz, 2H), 1.78 (t, J=7.9 Hz, 2H), 1.67-1.57 (m, 7H), 1.50-1.36 (m, 18H).

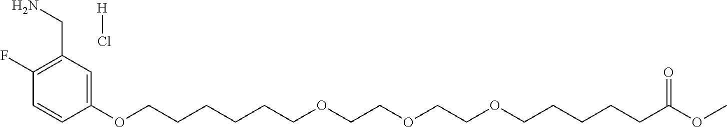

Compound 5. methyl 6-(2-(2-((6-(3-(aminomethyl)-4-fluorophenoxy)hexyl)oxy)ethoxy)ethoxy)hexanoate hydrochloride. Colorless oil 100%. LCMS (C18 column 20×2 mm, 2.5 μm, pore size 100 Å, water-acetonitrile+0.1% TFA, gradient 5 to 87% for 3 min, retention time 1.17 min). MS (ESI) m/z 458.6 [MH]+.

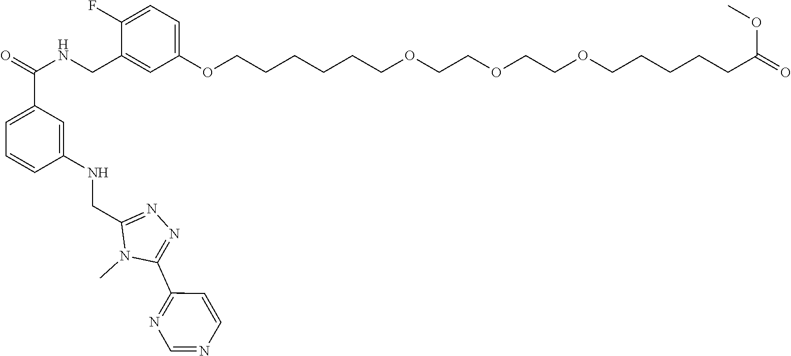 [SMILES: COC(=O)CCCCCOCCOCCOCCCCCCOc1ccc(F)c(CNC(=O)c2cccc(NCc3nnc(-c4ccncn4)n3C)c2)c1]

Compound 7. methyl 6-(2-(2-((6-(4-fluoro-3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)hexanoate. Colorless oil 61%. LCMS (C18 column 20×2 mm, 2.5 μm, pore size 100 Å, water-acetonitrile+0.1% TFA, gradient 5 to 87% for 3 min, retention time 1.57 min). MS (ESI) m/z 750.8 [MH]+. ¹H NMR (400 MHz, CDCl₃) δ 9.29 (s, 1H), 8.88 (d, J=7.1 Hz, 1H), 8.27 (d, J=7.1 Hz, 1H), 7.28-7.23 (m, 2H), 7.10 (d, J=7.2 Hz, 1H), 6.98-6.91 (m, 3H), 6.77-6.73 (m, 1H), 6.68-6.66 (m, 1H), 4.65-4.61 (m, 3H), 4.17 (s, 2H), 3.90 (t, J=7.9 Hz, 2H), 3.67-3.63 (m, 8H), 3.59-3.56 (m, 4H), 3.48-3.43 (m, 5H), 2.31 (t, J=7.9 Hz, 2H), 1.77 (t, J=7.9 Hz, 2H), 1.67-1.56 (m, 7H), 1.49-1.32 (m, 8H).

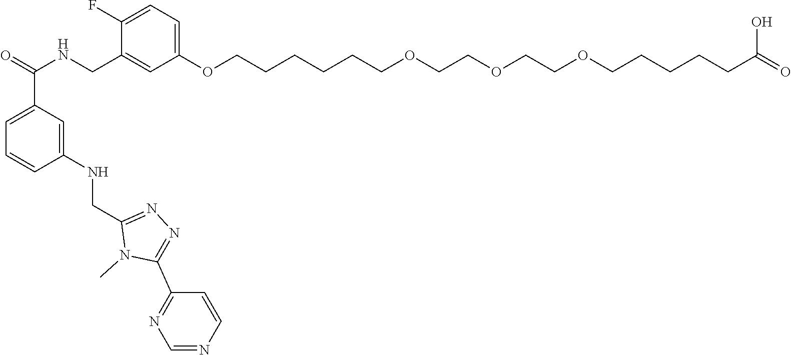 [SMILES: Cn1c(CNc2cccc(C(=O)NCc3cc(OCCCCCCOCCOCCOCCCCCC(=O)O)ccc3F)c2)nnc1-c1ccncn1]

Compound 8. 6-(2-(2-((6-(4-fluoro-3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)hexanoic acid. Colorless oil 99%. LCMS (C18 column 20×2 mm, 2.5 μm, pore size 100 Å, water-acetonitrile+0.1% TFA, gradient 5 to 87% for 3 min, retention time 1.40 min). MS (ESI) m/z 736.6 [MH]+.

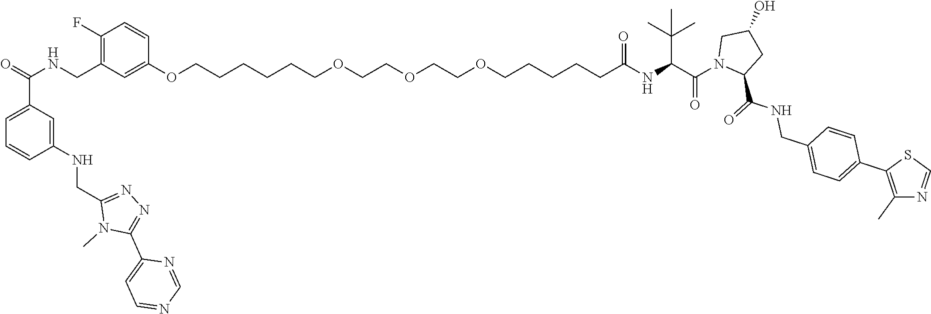 [SMILES: Cc1ncsc1-c1ccc(CNC(=O)[C@@H]2C[C@@H](O)CN2C(=O)[C@@H](NC(=O)CCCCCOCCOCCOCCCCCCOc2ccc(F)c(CNC(=O)c3cccc(NCc4nnc(-c5ccncn5)n4C)c3)c2)C(C)(C)C)cc1]

Compound 9. D88. (2S,4R)-1-((S)-2-(tert-butyl)-22-(4-fluoro-3-((3-(((4-methyl-5-(pyrimidin-4-yl)-4H-1,2,4-triazol-3-yl)methyl)amino)benzamido)methyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless oil 19%. LCMS (C18 column 100×4.6 mm, 5.0 μm, pore size 100 Å, water-acetonitrile+0.1% TFA, gradient 5 to 87% for 10 min, retention time 6.54 min). MS (ESI) m/z 1149.4 [MH]+. ¹H NMR (400 MHz, DMSO-d₆) δ 9.32 (s, 1H), 8.97-8.96 (m, 2H), 8.79 (t, J=6.1 Hz, 2H), 8.54 (t, J=6.1 Hz, 1H), 8.17 (d, J=6.1 Hz, 1H), 7.83 (d, J=7.1 Hz, 1H), 7.42-7.36 (m, 4H), 7.23 (s, 1H), 7.18 (t, J=7.1 Hz, 1H), 7.10 (d, J=7.2 Hz, 1H), 7.05 (d, J=7.2 Hz, 1H), 6.91 (d, J=7.2 Hz, 1H), 6.86-6.79 (m, 2H), 6.48 (t, J=7.2 Hz, 1H), 5.10 (d, J=5.2 Hz, 1H), 4.57 (d, J=6.2 Hz, 2H), 4.54 (d, J=7.2 Hz, 1H), 4.44-4.40 (m, 4H), 4.36-4.32 (m, 1H), 4.23-4.18 (m, 1H), 4.07 (s, 3H), 3.87 (t, J=7.9 Hz, 2H), 3.66-3.64 (m, 2H), 3.50-3.42 (m, 10H), 3.35-3.28 (m, 41H), 2.43 (s, 3H), 2.29-2.20 (m, 1H), 2.14-1.98 (m, 2H), 1.93-1.86 (m, 1H), 1.66-1.62 (m, 2H), 1.49-1.43 (m, 7H), 1.37-1.22 (m, 7H), 0.92 (s, 11H).

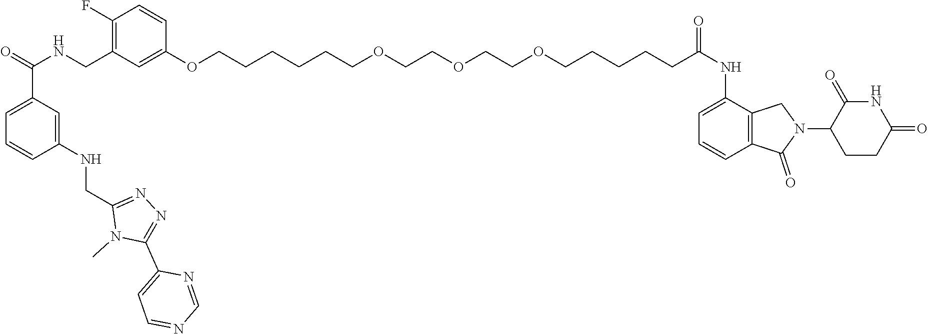 [SMILES: Cn1c(CNc2cccc(C(=O)NCc3cc(OCCCCCCOCCOCCOCCCCCC(=O)Nc4cccc5c4CN(C4CCC(=O)NC4=O)C5=O)ccc3F)c2)nnc1-c1ccncn1]

Compound 9. D66. N-(5-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)-2-fluorobenzyl)-3-(((4-methyl-5-(pyrimidin-4-yl)-4H-1,2,4-triazol-3-yl)methyl)amino)benzamide. Colorless oil 15%. LCMS (C18 column 100×4.6 mm, 5.0 μm, pore size 100 Å, water-acetonitrile+0.1% TFA, gradient 5 to 87% for 10 min, retention time 6.13 min). MS (ESI) m/z 978.0 [MH]+. ¹H NMR (400 MHz, DMSO-d₆) δ 11.00 (s, 1H), 9.74 (s, 1H), 9.32 (s, 1H), 8.96 (d, J=6.1 Hz, 1H), 8.80 (t, J=6.1 Hz, 1H), 8.16 (d, J=6.1 Hz, 1H), 7.81 (d, J=7.1 Hz, 1H), 7.50-7.45 (m, 2H), 7.23 (s, 1H), 7.18 (t, J=7.1 Hz, 1H), 7.10 (d, J=7.2 Hz, 1H), 7.05 (d, J=7.2 Hz, 1H), 6.91 (d, J=7.2 Hz, 1H), 6.85-6.79 (m, 2H), 6.48 (t, J=6.2 Hz, 1H), 5.14 (dd, J=7.2 Hz, J=2.2 Hz, 2H), 4.57 (d, J=6.2 Hz, 2H), 4.42 (d, J=6.2 Hz, 2H), 4.34 (d, J=7.2 Hz, 2H), 4.06 (s, 3H), 3.86 (t, J=7.2 Hz, 2H), 3.50-3.41 (m, 10H), 3.38-3.31 (m, 14H), 2.96-2.87 (m, 1H), 2.63-2.57 (m, 1H), 2.34 (t, J=6.2 Hz, 3H), 2.07-1.99 (m, 1H), 1.68-1.58 (m, 5H), 1.54-1.42 (m, 5H), 1.39-1.25 (m, 7H).

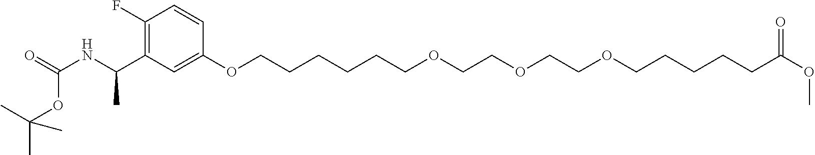 [SMILES: COC(=O)CCCCCOCCOCCOCCCCCCOc1ccc(F)c([C@@H](C)NC(=O)OC(C)(C)C)c1]

Compound 4. (R)-methyl 6-(2-(2-((6-(3-(1-((tert-butoxycarbonyl)amino)ethyl)-4-fluorophenoxy)hexyl)oxy)ethoxy)ethoxy)hexanoate. Colorless solid 48%. LCMS (C18 column 20×2 mm, 2.5 μm, pore size 100 Å, water-acetonitrile+0.1% TFA, gradient 5 to 87% for 3 min, retention time 2.14 min). MS (ESI) m/z 572.8 [MH]+. ¹H NMR (400 MHz, DMSO-d₆) δ 7.43 (br.d, J=8.4 Hz, 1H), 7.02 (t, J=9.53 Hz, 1H), 7.92 (dd, J=6.12, 3.06 Hz, 1H), 6.80-6.74 (m, 1H), 4.90-4.78 (m, 1H), 3.90 (t, J=6.36 Hz, 2H), 3.57 (s, 3H), 3.52-3.42 (m, 8H), 3.38 (dt, J=10.4, 6.5 Hz, 4H), 2.28 (t, J=7.4 Hz, 3H), 1.69 (pent, J=6.7 Hz, 2H), 1.56-1.44 (m, 6H), 1.36 (s, 9H), 1.31-1.22 (m, 6H).

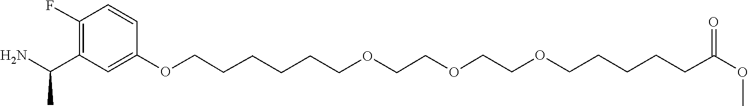 [SMILES: COC(=O)CCCCCOCCOCCOCCCCCCOc1ccc(F)c([C@@H](C)N)c1]

Compound 5. (R)-methyl 6-(2-(2-((6-(3-(1-aminoethyl)-4-fluorophenoxy)hexyl)oxy) ethoxy)ethoxy)hexanoate hydrochloride. Colorless solid 100%. LCMS (C18 column 20×2 mm, 2.5 μm, pore size 100 Å, water-acetonitrile+0.1% TFA, gradient 5 to 87% for 3 min, retention time 1.29 min). MS (ESI) m/z 472.6 [MH]+.

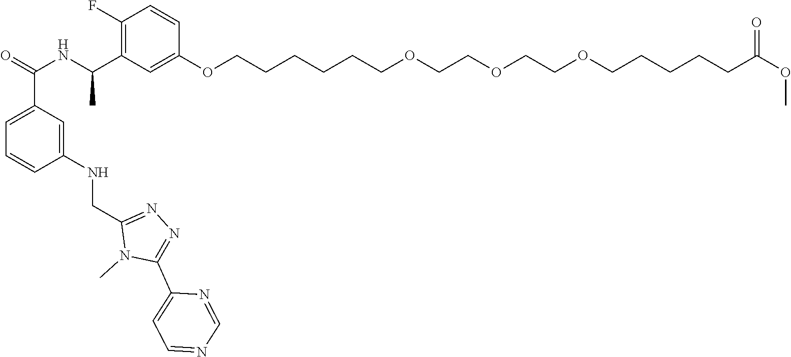 [SMILES: COC(=O)CCCCCOCCOCCOCCCCCCOc1ccc(F)c([C@@H](C)NC(=O)c2cccc(NCc3nnc(-c4ccncn4)n3C)c2)c1]

Compound 7. (R)-methyl 6-(2-(2-((6-(4-fluoro-3-(1-(3-(((4-methyl-5-(pyrimidin-4-yl)-4H-1,2,4-triazol-3-yl)methyl)amino)benzamido)ethyl)phenoxy)hexyl)oxy) ethoxy)ethoxy)hexanoate. Light brown 85%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 764.6 [MH]+.

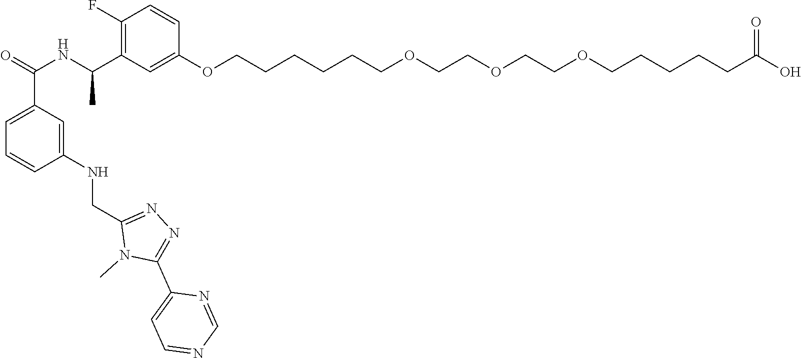 [SMILES: C[C@@H](NC(=O)c1cccc(NCc2nnc(-c3ccncn3)n2C)c1)c1cc(OCCCCCCOCCOCCOCCCCCC(=O)O)ccc1F]

Compound 8. (R)-6-(2-(2-((6-(4-fluoro-3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoic acid. Light brown solid 95%. LCMS (C18 column 20×2 mm, 2.5 μm, pore size 100 Å, water-acetonitrile+0.1% TFA, gradient 5 to 87% for 3 min, retention time 1.48 min). MS (ESI) m/z 750.4 [MH]+.

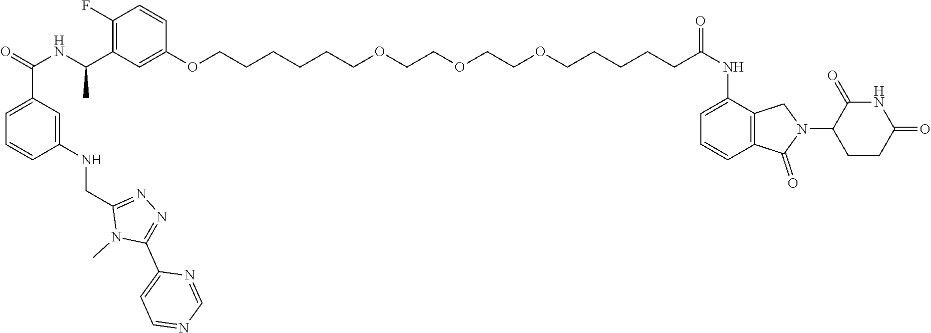 [SMILES: C[C@@H](NC(=O)c1cccc(NCc2nnc(-c3ccncn3)n2C)c1)c1cc(OCCCCCCOCCOCCOCCCCCC(=O)Nc2cccc3c2CN(C2CCC(=O)NC2=O)C3=O)ccc1F]

Compound 9. D54. N-((1R)-1-(5-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)-2-fluorophenyl)ethyl)-3-(((4-methyl-5-(pyrimidin-4-yl)-4H-1,2,4-triazol-3-yl)methyl)amino)benzamide. Colorless solid 15%. LCMS (C18 column 100×4.6 mm, 5.0 μm, pore size 100 Å, water-acetonitrile+0.1% TFA, gradient 5 to 87% for 10 min, retention time 6.33 min). MS (ESI) m/z 992.0 [MH]+. ¹H NMR (400 MHz, DMSO-d₆) δ 11.0 (s, 1H), 9.75 (s, 1H), 9.32 (d, J=1.22 Hz, 1H), 8.97 (d, J=5.38 Hz, 1H), 8.62 (d, J=7.82 Hz, 1H), 8.17 (dd, J=5.25, 1.46 Hz, 1H), 7.81 (dd, J=7.46, 1.71 Hz, 1H), 7.52-7.44 (m, 2H), 7.21-7.16 (m, 2H), 7.14-7.09 (m, 1H), 7.07-6.97 (m, 2H), 6.93-6.88 (m, 1H), 6.80-6.75 (m, 1H), 6.48 (t, J=5.5 Hz, 1H), 5.37-5.27 (m, 1H), 5.14 (dd, J=13.2, 5.02 Hz, 1H), 4.57 (d, J=5.38 Hz, 2H), 4.36 (dd, J=7.70 Hz, 2H), 4.07 (s, 3H), 3.88 (t, J=6.24 Hz, 2H), 3.50-3.40 (m, 8H), 3.39-3.32 (m, 4H), 2.97-2.86 (m, 1H), 2.69-2.53 (m, 2H), 2.34 (t, J=7.33 Hz, 3H), 2.07-1.97 (m, 1H), 1.70-1.25 (m, 18H).

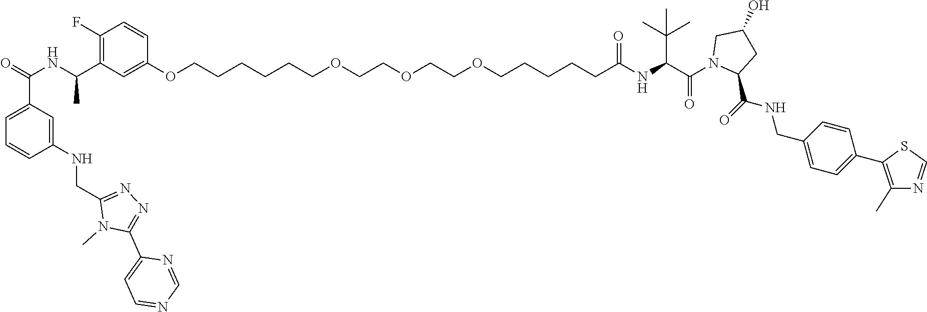 [SMILES: Cc1ncsc1-c1ccc(CNC(=O)[C@@H]2C[C@@H](O)CN2C(=O)[C@@H](NC(=O)CCCCCOCCOCCOCCCCCCOc2ccc(F)c([C@@H](C)NC(=O)c3cccc(NCc4nnc(-c5ccncn5)n4C)c3)c2)C(C)(C)C)cc1]

Compound 9. D75. (2S,4R)-1-((S)-2-(tert-butyl)-22-(4-fluoro-3-((R)-1-(3-(((4-methyl-5-(pyrimidin-4-yl)-4H-1,2,4-triazol-3-yl)methyl)amino)benzamido)ethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless solid 50%. LCMS (C18 column 100×4.6 mm, 5.0 μm, pore size 100 Å, water-acetonitrile+0.1% TFA, gradient 5 to 87% for 10 min, retention time 6.68 min). MS (ESI) m/z 1163.3 [MH]+. ¹H NMR (400 MHz, DMSO-d₆) δ 9.33 (d, J=1.22 Hz, 1H), 9.00-8.94 (m, 2H), 8.62 (d, J=8.20 Hz, 1H), 8.56 (t, J=5.50 Hz, 1H), 8.17 (dd, J=5.26, 1.35 Hz, 1H), 7.84 (d, J=9.41 Hz, 1H), 7.39 (q, J=8.31 Hz, 4H), 7.22-7.15 (m, 2H), 7.13-7.09 (m, 1H), 7.07-6.97 (m, 2H), 6.93-6.89 (m, 1H), 6.80-6.75 (m, 1H), 6.48 (t, J=5.5 Hz, 1H), 5.37-5.28 (m, 1H), 5.11 (d, J=3.55 Hz, 1H), 4.60-4.51 (m, 3H), 4.47-4.39 (m, 2H), 4.34 (br.s, 1H), 4.25-4.17 (m, 1H), 4.07 (s, 3H), 3.88 (t, J=6.50 Hz, 2H), 3.70-3.60 (m, 2H), 3.50-3.40 (m, 8H), 3.37-3.32 (m, 4H), 2.44 (s, 3H), 2.30-2.20 (m, 1H), 2.15-1.99 (m, 2H), 1.55-1.19 (m, 16H), 0.93 (s, 9H).

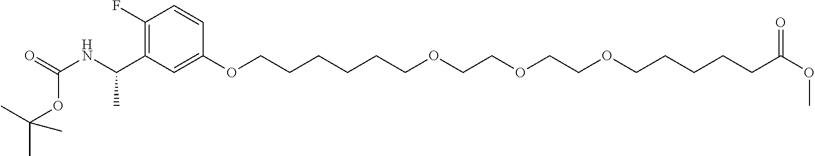 [SMILES: COC(=O)CCCCCOCCOCCOCCCCCCOc1ccc(F)c([C@H](C)NC(=O)OC(C)(C)C)c1]

Compound 4. (S)-methyl 6-(2-(2-((6-(3-(1-((tert-butoxycarbonyl)amino)ethyl)-4-fluorophenoxy)hexyl)oxy)ethoxy)ethoxy)hexanoate. Colorless solid 57%. LCMS (C18 column 20×2 mm, 2.5 μm, pore size 100 Å, water-acetonitrile+0.1% TFA, gradient 5 to 87% for 3 min, retention time 2.14 min). MS (ESI) m/z 572.7 [MH]+. ¹H NMR (400 MHz, DMSO-d₆) δ 7.42 (br.d, J=8.68 Hz, 1H), 7.02 (t, J=9.04 Hz, 1H), 7.92 (dd, J=6.12, 3.06 Hz, 1H), 6.80-6.74 (m, 1H), 4.89-4.78 (m, 1H), 3.90 (t, J=6.36 Hz, 2H), 3.57 (s, 3H), 3.52-3.42 (m, 8H), 3.38 (dt, J=10.4, 6.48 Hz, 4H), 2.28 (t, J=7.34 Hz, 3H), 1.69 (pent, J=6.7 Hz, 2H), 1.57-1.44 (m, 6H), 1.36 (s, 9H), 1.31-1.22 (m, 6H).

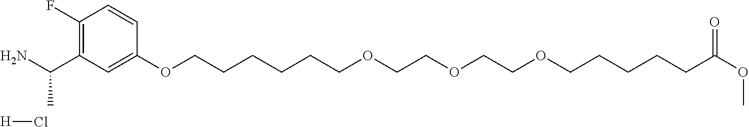

Compound 5. (S)-methyl 6-(2-(2-((6-(3-(1-aminoethyl)-4-fluorophenoxy)hexyl)oxy)ethoxy)ethoxy)hexanoate hydrochloride. Colorless solid 100%. LCMS (C18 column 20×2 mm, 2.5 μm, pore size 100 Å, water-acetonitrile+0.1% TFA, gradient 5 to 87% for 3 min, retention time 1.27 min). MS (ESI) m/z 472.6 [MH]+.

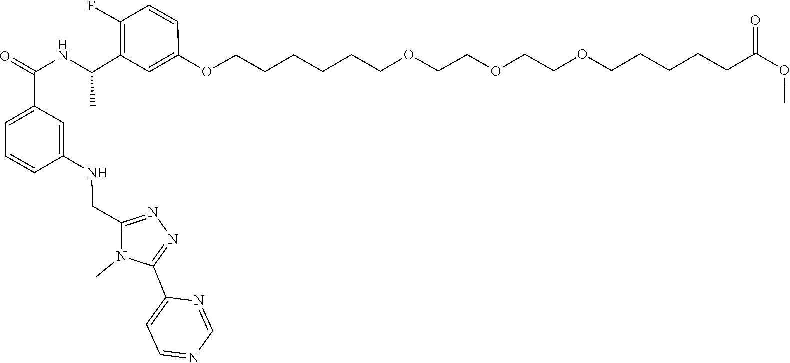 [SMILES: COC(=O)CCCCCOCCOCCOCCCCCCOc1ccc(F)c([C@H](C)NC(=O)c2cccc(NCc3nnc(-c4ccncn4)n3C)c2)c1]

Compound 7. (S)-methyl 6-(2-(2-((6-(4-fluoro-3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoate. Light brown solid 83%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 764.7 [MH]+.

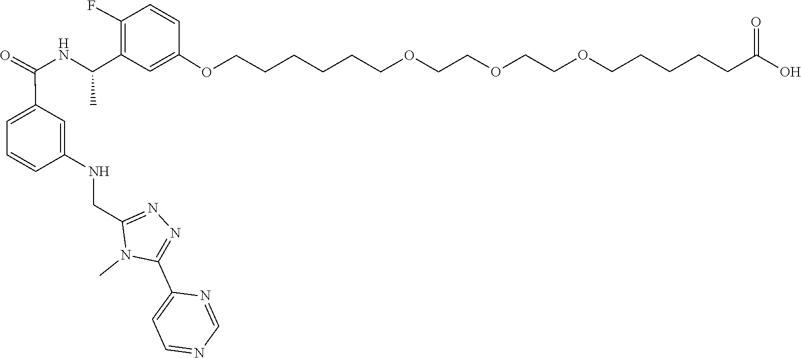 [SMILES: C[C@H](NC(=O)c1cccc(NCc2nnc(-c3ccncn3)n2C)c1)c1cc(OCCCCCCOCCOCCOCCCCCC(=O)O)ccc1F]

Compound 8. (S)-6-(2-(2-((6-(4-fluoro-3-(1-(3-(((4-methyl-5-(pyrimidin-4-yl)-4H-1,2,4-triazol-3-yl)methyl)amino)benzamido)ethyl)phenoxy)hexyl)oxy)ethoxy)ethoxy)hexanoic acid. Light brown solid 94%. LCMS (C18 column 20×2 mm, 2.5 μm, pore size 100 Å, water-acetonitrile+0.1% TFA, gradient 5 to 87% for 3 min, retention time 1.48 min). MS (ESI) m/z 750.7 [MH]+.

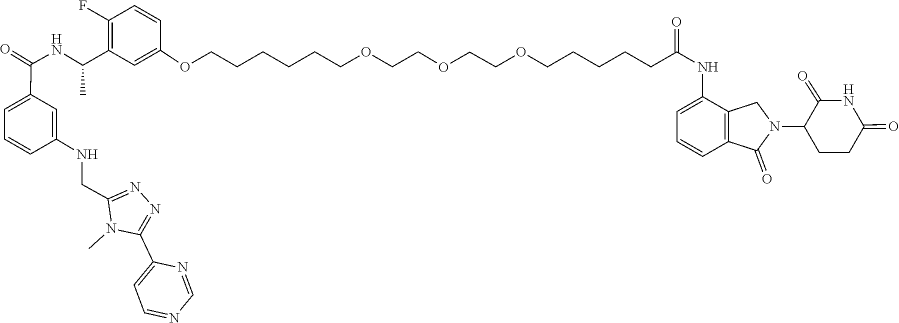 [SMILES: C[C@H](NC(=O)c1cccc(NCc2nnc(-c3ccncn3)n2C)c1)c1cc(OCCCCCCOCCOCCOCCCCCC(=O)Nc2cccc3c2CN(C2CCC(=O)NC2=O)C3=O)ccc1F]

Compound 9. D53. N-((1S)-1-(5-((6-(2-(2-((6-((2-(2,6-dioxopiperidin-3-yl)-1-oxoisoindolin-4-yl)amino)-6-oxohexyl)oxy)ethoxy)ethoxy)hexyl)oxy)-2-fluorophenyl)ethyl)-3-(((4-methyl-5-(pyrimidin-4-yl)-4H-1,2,4-triazol-3-yl)methyl)amino)benzamide. Colorless solid 35%. LCMS (C18 column 100×4.6 mm, 5.0 μm, pore size 100 Å, water-acetonitrile+0.1% TFA, gradient 5 to 87% for 10 min, retention time 6.33 min). MS (ESI) m/z 992.0 [MH]+. ¹H NMR (400 MHz, DMSO-d₆) δ 11.0 (s, 1H), 9.75 (s, 1H), 9.31 (d, J=1.21 Hz, 1H), 8.97 (d, J=5.40 Hz, 1H), 8.61 (d, J=7.79 Hz, 1H), 8.17 (dd, J=5.25, 1.46 Hz, 1H), 7.81 (dd, J=7.46, 1.71 Hz, 1H), 7.52-7.44 (m, 2H), 7.21-7.16 (m, 2H), 7.14-7.09 (m, 1H), 7.07-6.97 (m, 2H), 6.93-6.88 (m, 1H), 6.81-6.75 (m, 1H), 6.48 (t, J=5.5 Hz, 1H), 5.37-5.27 (m, 1H), 5.14 (dd, J=13.2, 5.03 Hz, 1H), 4.57 (d, J=5.38 Hz, 2H), 4.36 (dd, J=7.60 Hz, 2H), 4.07 (s, 3H), 3.88 (t, J=6.24 Hz, 2H), 3.51-3.40 (m, 8H), 3.39-3.32 (m, 4H), 2.97-2.86 (m, 1H), 2.69-2.53 (m, 2H), 2.34 (t, J=7.33 Hz, 3H), 2.07-1.97 (m, 1H), 1.70-1.25 (m, 18H).

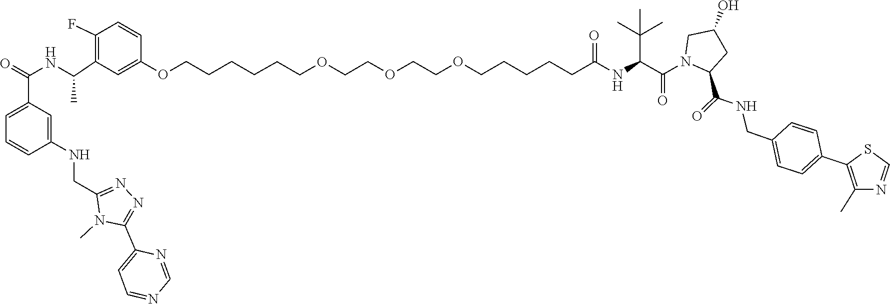 [SMILES: Cc1ncsc1-c1ccc(CNC(=O)[C@@H]2C[C@@H](O)CN2C(=O)[C@@H](NC(=O)CCCCCOCCOCCOCCCCCCOc2ccc(F)c([C@H](C)NC(=O)c3cccc(NCc4nnc(-c5ccncn5)n4C)c3)c2)C(C)(C)C)cc1]

Compound 9. D74. (2S,4R)-1-((S)-2-(tert-butyl)-22-(4-fluoro-3-((S)-1-(3-(((4-methyl-5-(pyrimidin-4-yl)-4H-1,2,4-triazol-3-yl)methyl)amino)benzamido)ethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. Colorless solid 50%. LCMS (C18 column 100×4.6 mm, 5.0 μm, pore size 100 Å, water-acetonitrile+0.1% TFA, gradient 5 to 87% for 10 min, retention time 6.77 min). MS (ESI) m/z 1163.3 [MH]+. ¹H NMR (400 MHz, DMSO-d₆) δ 9.33 (d, J=1.20 Hz, 1H), 9.00-8.94 (m, 2H), 8.62 (d, J=8.20 Hz, 1H), 8.56 (t, J=5.49 Hz, 1H), 8.17 (dd, J=5.26, 1.35 Hz, 1H), 7.84 (d, J=9.41 Hz, 1H), 7.39 (q, J=8.31 Hz, 4H), 7.22-7.15 (m, 2H), 7.12-7.09 (m, 1H), 7.07-6.97 (m, 2H), 6.92-6.89 (m, 1H), 6.80-6.75 (m, 1H), 6.49 (t, J=5.51 Hz, 1H), 5.37-5.28 (m, 1H), 5.11 (d, J=3.55 Hz, 1H), 4.60-4.51 (m, 3H), 4.48-4.39 (m, 2H), 4.34 (br.s, 1H), 4.25-4.17 (m, 1H), 4.07 (s, 3H), 3.88 (t, J=6.51 Hz, 2H), 3.70-3.60 (m, 2H), 3.50-3.40 (m, 8H), 3.39-3.32 (m, 4H), 2.44 (s, 3H), 2.30-2.20 (m, 1H), 2.15-1.99 (m, 2H), 1.55-1.19 (m, 16H), 0.93 (s, 9H).

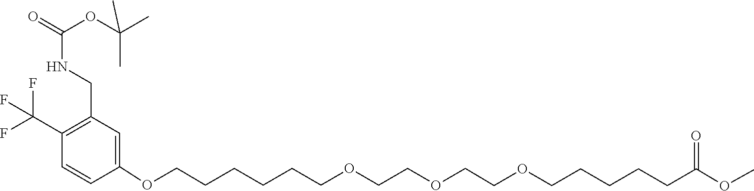 [SMILES: COC(=O)CCCCCOCCOCCOCCCCCCOc1ccc(C(F)(F)F)c(CNC(=O)OC(C)(C)C)c1]

Compound 4. methyl 6-(2-(2-((6-(3-(((tert-butoxycarbonyl)amino)methyl)-4-(trifluoromethyl)phenoxy)hexyl)oxy)ethoxy)ethoxy)hexanoate. Colorless oil 60%. LCMS (C18 column 20×2 mm, 2.5 μm, pore size 100-Å, water-acetonitrile+0.1% TFA, gradient 5 to 87% for 3 min, retention time 2.13 min). MS (ESI) m/z 608.7 [MH]+.

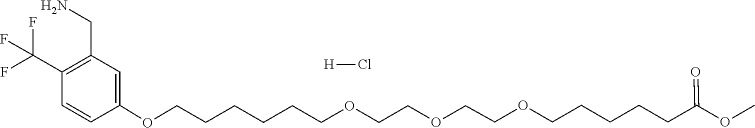

Compound 5. methyl 6-(2-(2-((6-(3-(aminomethyl)-4-(trifluoromethyl)phenoxy)hexyl)oxy)ethoxy)ethoxy)hexanoate. 100%. LCMS (C18 column 20×2 mm, 2.5 μm, pore size 100-Å, water-acetonitrile+0.1% TFA, gradient 5 to 87% for 3 min, retention time 1.21 min). MS (ESI) m/z 508.6 [MH]+.

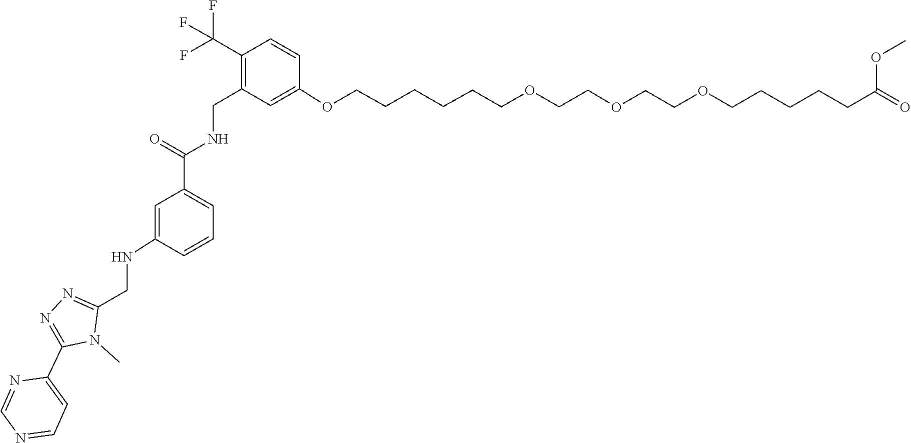 [SMILES: COC(=O)CCCCCOCCOCCOCCCCCCOc1ccc(C(F)(F)F)c(CNC(=O)c2cccc(NCc3nnc(-c4ccncn4)n3C)c2)c1]

Compound 7. methyl 6-(2-(2-((6-(3-((3-(((4-methyl-5-(pyrimidin-4-yl)-4H-1,2,4-triazol-3-yl)methyl)amino)benzamido)methyl)-4-(trifluoromethyl)phenoxy)hexyl)oxy)ethoxy)ethoxy)hexanoate. 13%. LCMS (C18 column 20×2 mm, 2.5 μm, pore size 100-Å, water-acetonitrile+0.1% TFA, gradient 5 to 87% for 3 min, retention time 1.82 min). MS (ESI) m/z 800.8 [MH]+.

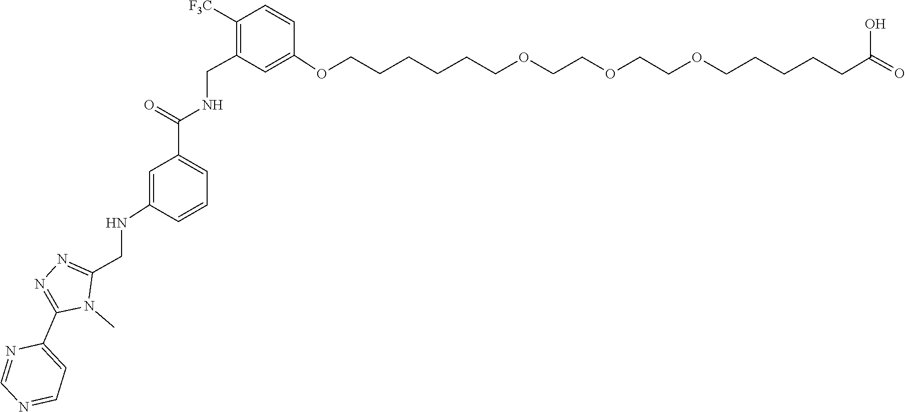 [SMILES: Cn1c(CNc2cccc(C(=O)NCc3cc(OCCCCCCOCCOCCOCCCCCC(=O)O)ccc3C(F)(F)F)c2)nnc1-c1ccncn1]

Compound 8. 6-(2-(2-((6-(3-((3-(((4-methyl-5-(pyrimidin-4-yl)-4H-1,2,4-triazol-3-yl)methyl)amino)benzamido)methyl)-4-(trifluoromethyl)phenoxy)hexyl)oxy)ethoxy)ethoxy)hexanoic acid. 95%. LCMS (C18 column 20×2 mm, 2.5 μm, pore size 100-Å, water-acetonitrile+0.1% TFA, gradient 5 to 87% for 3 min, retention time 1.6 min). MS (ESI) m/z 786.9 [MH]+.

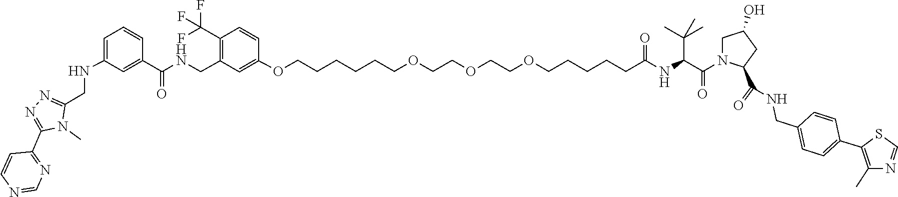 [SMILES: Cc1ncsc1-c1ccc(CNC(=O)[C@@H]2C[C@@H](O)CN2C(=O)[C@@H](NC(=O)CCCCCOCCOCCOCCCCCCOc2ccc(C(F)(F)F)c(CNC(=O)c3cccc(NCc4nnc(-c5ccncn5)n4C)c3)c2)C(C)(C)C)cc1]

Compound 9. D92. (2S,4R)-1-((S)-2-(tert-butyl)-22-(3-((3-(((4-methyl-5-(pyrimidin-4-yl)-4H-1,2,4-triazol-3-yl)methyl)amino)benzamido)methyl)-4-(trifluoromethyl)phenoxy)-4-oxo-10,13,16-trioxa-3-azadocosan-1-oyl)-4-hydroxy-N-(4-(4-methylthiazol-5-yl)benzyl)pyrrolidine-2-carboxamide. White powder 38%. LCMS (C18 column 100×4.6 mm, 5.0 μm, pore size 100-Å, water-acetonitrile+0.1% TFA, gradient 5 to 87% for 10 min, retention time 6.94 min). MS (ESI) m/z 1199.4 [MH]+. ¹H NMR (400 MHz, DMSO-d₆) δ 9.33 (s, 1H), 8.97 (d, J=5.0 Hz, 2H), 8.89 (s, J=5.4 Hz, 1H), 8.55 (t, J=5.4 Hz, 1H), 8.18 (d, J=5.2 Hz, 1H), 7.83 (d, J=9.3 Hz, 1H), 7.63 (d, J=8.5 Hz, 1H), 7.39 (q, J=8.3 Hz, 4H), 7.27 (s, 1H), 7.21 (t, J=7.7 Hz, 1H), 7.14 (d, J=7.8 Hz, 1H), 7.02-6.88 (m, 3H), 6.51 (t, J=5.7 Hz, 1H), 5.11 (d, J=3.4 Hz, 1H), 4.65-4.50 (m, 4H), 4.48-4.38 (m, 2H), 4.34 (s, 1H), 4.26-4.17 (m, 1H), 4.07 (s, 3H), 3.98 (t, J=6.5 Hz, 2H), 3.72-3.58 (m, 2H), 3.51-3.39 (m, 8H), 3.35-3.32 (m, 3H), 2.44 (s, 3H), 2.30-2.19 (m, 1H), 2.16-1.96 (m, 2H), 1.95-1.83 (m, 1H), 1.73-1.59 (m, 2H), 1.56-1.40 (m, 6H), 1.40-1.16 (m, 8H), 0.92 (s, 9H).

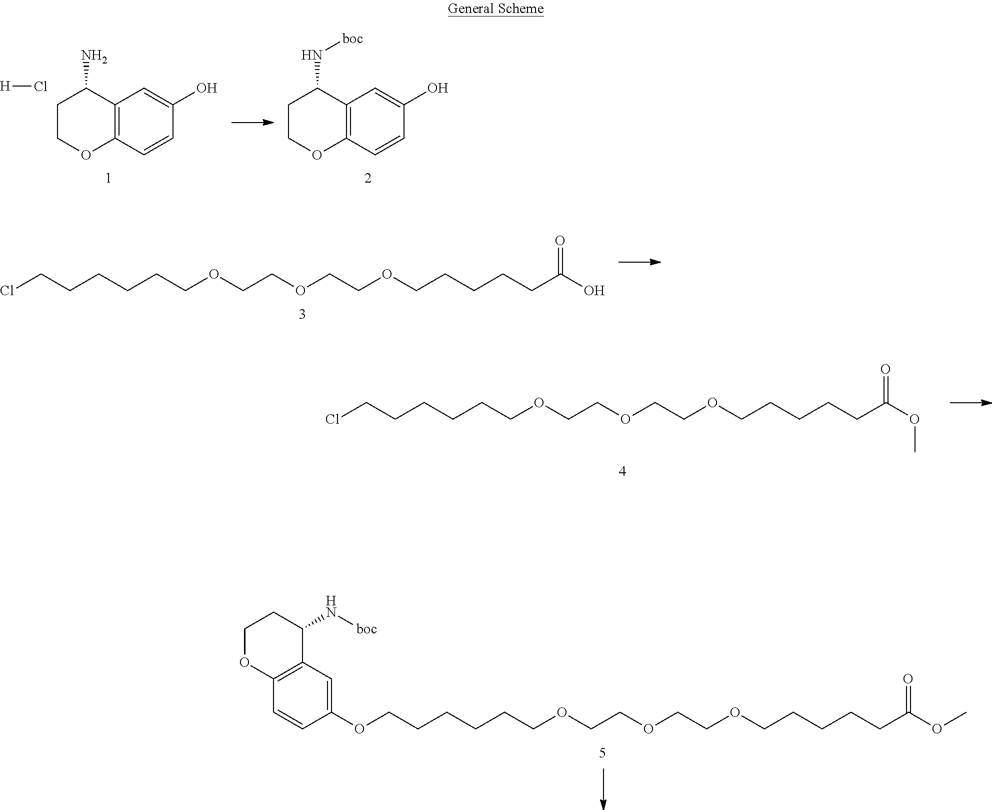

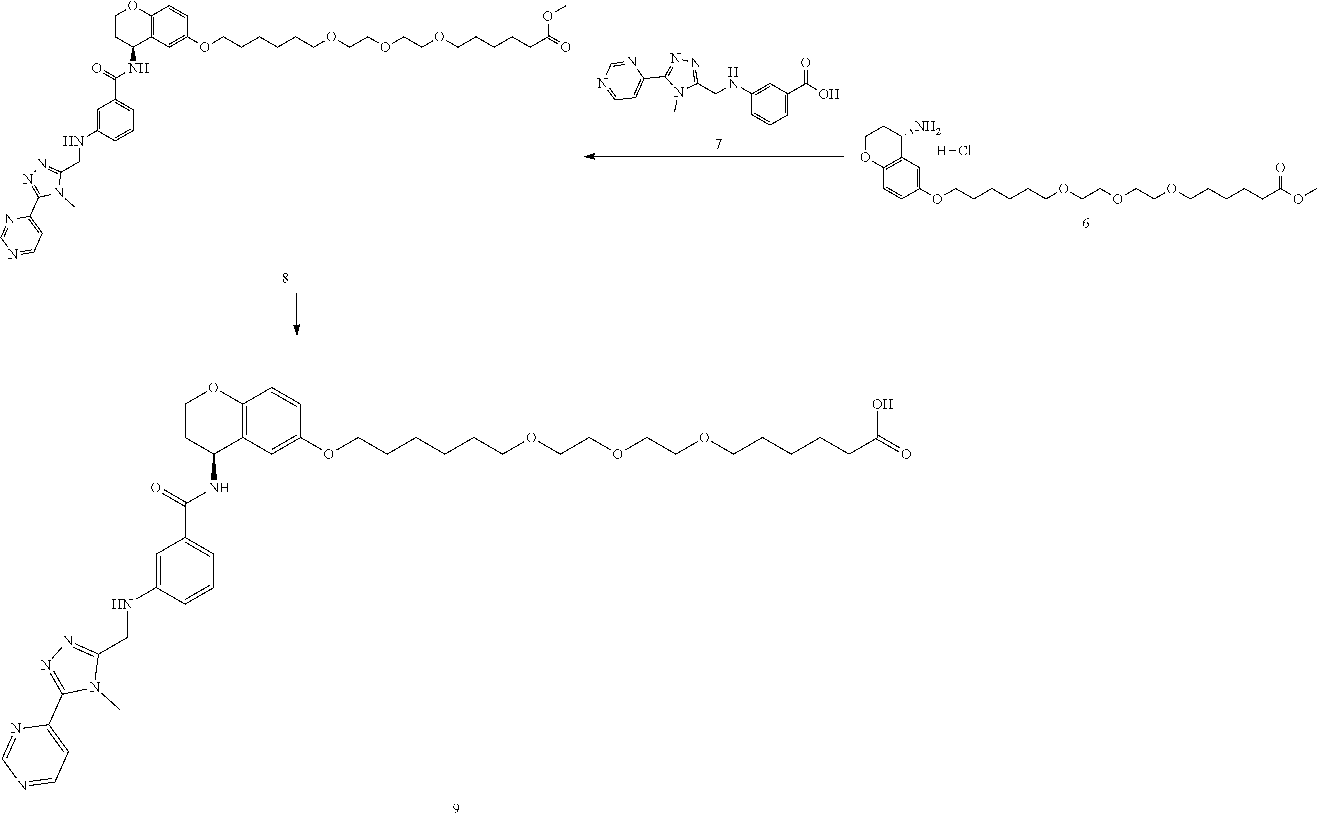

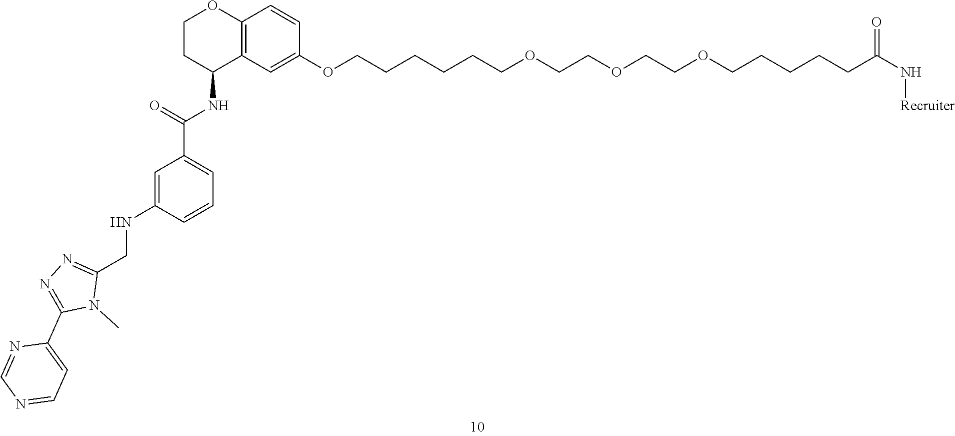

### General Procedures

Compound 4. Methyl 6-(2-(2-((6-chlorohexyl)oxy)ethoxy)ethoxy)hexanoate. To a solution of compound 3 (3.3 mmol, 1 eq) in dry MeOH (15 mL) was added dropwise SOCl₂ (0.48 ml, 6.6 mmol, 2 eq) at 0° C. The mixture was stirred for 12 h at r.t. and then evaporated to dryness to give the crude product 4 which no need additional purification. Colorless oil, yield 98%. ¹H NMR (400 MHz, DMSO) δ 3.61 (t, J=6.5 Hz, 2H), 3.57 (s, 3H), 3.53-3.47 (m, 4H), 3.45 (d, J=3.5 Hz, 4H), 3.36 (dd, J=9.7, 6.2 Hz, 4H), 2.28 (t, J=7.3 Hz, 2H), 1.69 (dd, J=13.9, 6.8 Hz, 2H), 1.59-1.43 (m, 6H), 1.41-1.19 (m, 6H).

Compound 5. Compound 4 (2.5 mmol, 1 eq), compound 2 (2.75 mmol, 1.1 eq), K₂CO₃ (5.0 mmol, 2 eq), KI (0.25 mmol, 0.1 eq) in dry acetonitrile (50 mL) were stirred for 12 h at reflux. The precipitate was filtered off, washed with acetonitrile. The solvent was evaporated to dryness. The residue was purified by silica gel flash column chromatography using chloroform and methanol as an eluent to afford the desired product 5.

Compound 6. A solution of compound 5 (2.5 mmol, 1 eq) in dry DCM (50 mL) with 12.5 ml 3 M HCl in dioxane (5 eq) were stirred for 24 h at r.t. The mixture was evaporated to dryness to give the crude product 6 which was used in the next step without additional purification.

Compound 8. Compound 7 (1.2 mmol, 1 eq), compound 6 (1.2 mmol, 1 eq), HOBt (1.2 mmol, 1 eq), EDCI (1.32 mmol, 1.1 eq) and DIPEA (4.8 mmol, 4 eq) in dry DCM (30 ml) were stirred for 12 h at r.t. The reaction mixture was washed with water (3*10 ml), brine (2×10 mL), dried on activated Na₂SO₄, and concentrated in a rotary evaporator under reduced pressure at ambient temperature. The residue was purified by silica gel flash column chromatography using chloroform and methanol as an eluent to afford the desired product 8.

Compound 9. To a solution of compound 8 (1.2 mmol, 1 eq) in MeOH (20 mL) was added a solution KOH (3.6 mmol, 3 eq) in 3 ml H₂O. The mixture was stirred for 12-36 h at r.t. and then MeOH evaporated. To the residue was added 15 ml of H₂O and the mixture was acidified to pH 5 with 1N aq. HCl and aqueous phase was extracted with 4*15 ml of DCM. Combined organic phases were washed with brine, dried over Na₂SO₄, and evaporated. The product 9 was used in the next step without additional purification.

Compound 10. Compound 9 (0.1 mmol, 1 eq), recruiter (0.1 mmol, 1 eq), TBTU (0.2 mmol, 2 eq) and DIPEA (0.4 mmol, 4 eq) in dry pyridine (3 ml) were stirred for 12 h at r.t. The mixture was evaporated to dryness. The residue was purified by HPLC in neutral conditions (YMC-Pack ODS-AQ 250×20 mml, S-10 μm, 12 nm, gradient water-acetonitrile).

### Syntheses According to the General Procedures

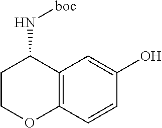

Compound 2. (S)-tert-butyl 6-hydroxychroman-4-ylcarbamate. To an ice cold suspension of amine hydrochloride 1 (1 g, 4.96 mmol) in DCM (20 ml), di-tert-butyl dicarbonate (1.2 g, 5.49 mmol) was added, followed by triethylamine (0.8 ml, 0.58 g, 5.74 mmol). The mixture was stirred at ambient temperature for overnight and quenched with water (10 ml). Organic phase was separated; aqueous phase was extracted with DCM (2 by 10 ml). Combined organic phases were dried over MgSO4 and the solvent was stripped off. The residue was crystallized from DCM-hexane to afford 1.3 g (98%) of compound 2. ¹H NMR (400 MHz, CDCl₃) δ 6.75 (s, 1H), 6.71-6.69 (m, 2H), 5.81 (s, 1H), 4.90 (d, J=8.1 Hz, 1H), 4.82 (s, 1H), 4.21-4.16 (m, 1H), 4.12-4.07 (m, 1H), 2.23-2.05 (m, 1H), 2.05-1.91 (m, 1H), 1.51 (s, 9H).

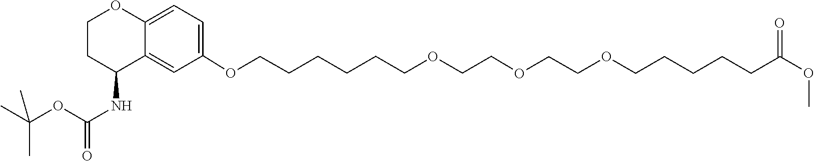 [SMILES: COC(=O)CCCCCOCCOCCOCCCCCCOc1ccc2c(c1)[C@@H](NC(=O)OC(C)(C)C)CCO2]

Compound 5. (S)-methyl 6-(2-(2-(6-(4-(tert-butoxycarbonylamino)chroman-6-yloxy)hexyloxy)-ethoxy)ethoxy)hexanoate. Colorless oil 99%. LCMS (C18 column 20×2 mm, 2.5 μm, pore size 100 Å, water-acetonitrile+0.1% TFA, gradient 5 to 87% for 3 min, retention time 2.06 min). MS (ESI) m/z 582.5 [MH]+.

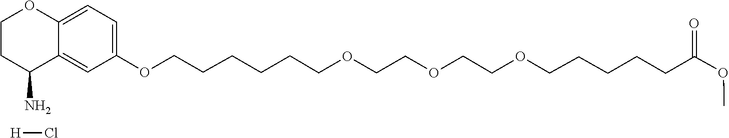

Compound 6. (S)-methyl 6-(2-(2-(6-(4-aminochroman-6-yloxy)hexyloxy)ethoxy)ethoxy)-hexanoate hydrochloride. Colorless crystals 100%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 482.5 [MH]+.

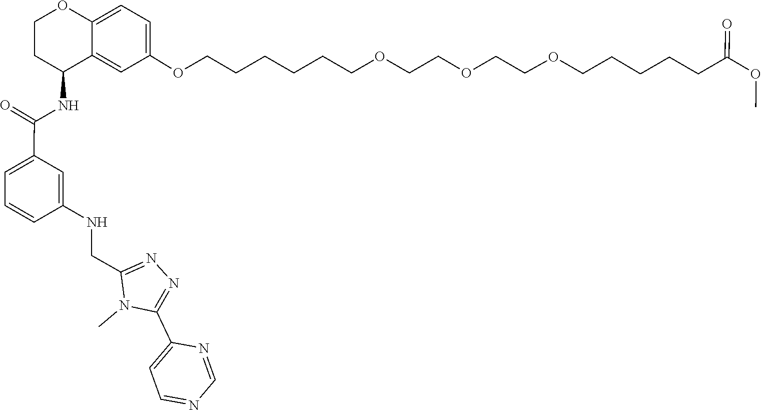 [SMILES: COC(=O)CCCCCOCCOCCOCCCCCCOc1ccc2c(c1)[C@@H](NC(=O)c1cccc(NCc3nnc(-c4ccncn4)n3C)c1)CCO2]

Compound 8. (S)-methyl 6-(2-(2-(6-(4-(3-((4-methyl-5-(pyrimidin-4-yl)-4H-1,2,4-triazol-3-yl)methylamino)benzamido)chroman-6-yloxy)hexyloxy)ethoxy)ethoxy)hexanoate. Colorless oil 63%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 774.5 [MH]+. ¹H NMR (400 MHz, CDCl₃) δ 9.29 (d, J=1.2 Hz, 1H), 8.89 (d, J=5.2 Hz, 1H), 8.29 (dd, J=5.3, 1.3 Hz, 1H), 7.31 (s, 1H), 7.29-7.22 (m, 1H), 7.10 (d, J=7.7 Hz, 1H), 7.01-6.90 (m, 1H), 6.79 (s, 3H), 6.49 (d, J=7.3 Hz, 1H), 5.32 (d, J=5.9 Hz, 1H), 4.71 (s, 1H), 4.60 (d, J=5.0 Hz, 2H), 4.26 (d, J=6.0 Hz, 1H), 4.17 (d, J=13.8 Hz, 4H), 3.86 (t, J=6.5 Hz, 2H), 3.75 (t, J=6.6 Hz, 6H), 3.66 (s, 3H), 3.65-3.62 (m, 4H), 3.58-3.57 (m, 4H), 3.46-3.43 (m, 4H), 2.30 (t, J=7.5 Hz, 2H), 2.16 (d, J=5.8 Hz, 1H), 1.90-1.85 (m, 2H), 1.75-1.71 (m, 1H), 1.67-1.58 (m, 4H), 1.45-1.37 (m, 2H).

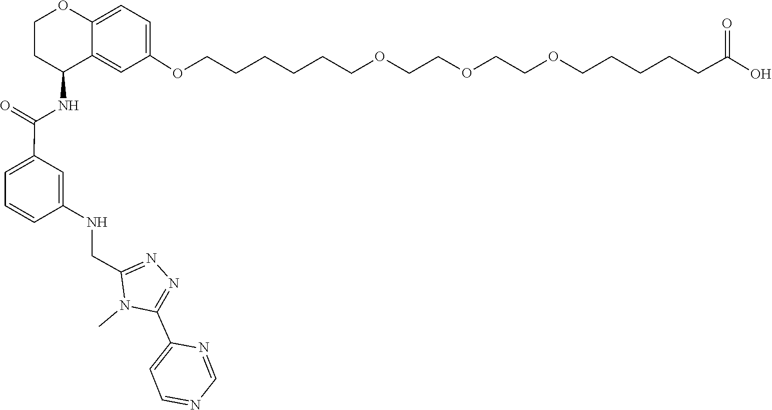 [SMILES: Cn1c(CNc2cccc(C(=O)N[C@H]3CCOc4ccc(OCCCCCCOCCOCCOCCCCCC(=O)O)cc43)c2)nnc1-c1ccncn1]

Compound 9. (S)-6-(2-(2-(6-(4-(3-((4-methyl-5-(pyrimidin-4-yl)-4H-1,2,4-triazol-3-yl)methyl-amino)benzamido)chroman-6-yloxy)hexyloxy)ethoxy)ethoxy)hexanoic acid. Colorless oil 87%. LCMS (C18 column 20×2 mm, 2.5 μm, pore size 100 Å, water-acetonitrile+0.1% TFA, gradient 5 to 87% for 3 min, retention time 1.43 min). MS (ESI) m/z 760.5 [MH]+.

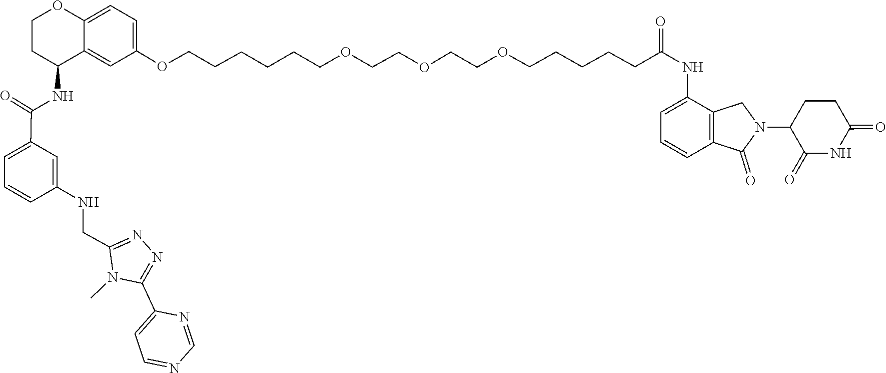 [SMILES: Cn1c(CNc2cccc(C(=O)N[C@H]3CCOc4ccc(OCCCCCCOCCOCCOCCCCCC(=O)Nc5cccc6c5CN(C5CCC(=O)NC5=O)C6=O)cc43)c2)nnc1-c1ccncn1]

Compound 10. D156. N-((4S)-6-(6-(2-(2-(6-(2-(2,6-dioxopiperidin-3-yl)-1-oxoisoindolin-4-ylamino)-6-oxohexyloxy)ethoxy)ethoxy)hexyloxy)chroman-4-yl)-3-((4-methyl-5-(pyrimidin-4-yl)-4H-1,2,4-triazol-3-yl)methylamino)benzamide. Colorless oil 43%. LCMS (C18 column 100×4.6 mm, 5.0 μm, pore size 100 Å, water-acetonitrile+0.1% TFA, gradient 5 to 87% for 10 min, retention time 6.06 min). MS (ESI) m/z 1003.0 [MH]+. ¹H NMR (400 MHz, DMSO-d₆) δ 10.99 (s, 1H), 9.73 (s, 1H), 9.33 (s, 1H), 8.97 (d, J=5.3 Hz, 1H), 8.63 (d, J=8.2 Hz, 1H), 8.17 (d, J=5.3 Hz, 1H), 7.81 (dd, J=6.9, 1.6 Hz, 1H), 7.51-7.45 (m, 2H), 7.26 (br, 1H), 7.18-7.11 (m, 2H), 6.91 (d, J=8.7 Hz, 1H), 6.75-6.67 (m, 3H), 6.44 (t, J=5.6 Hz, 1H), 5.25-5.20 (m, 1H), 5.13 (dd, J=13.4, 5.0 Hz, 1H), 4.57 (d, J=5.6 Hz, 2H), 4.41-4.30 (m, 2H), 4.30-4.26 (m, 1H), 4.17-4.11 (m, 1H), 4.07 (s, 3H), 3.81 (t, J=6.4 Hz, 2H), 3.48-3.40 (m, 9H), 3.37 (t, J=6.5 Hz, 2H), 3.34 (t, J=6.4 Hz, 1H), 2.95-2.87 (m, 1H), 2.67-2.57 (m 1H), 2.38-2.28 (m, 3H), 2.09-1.98 (m, 3H), 1.64-1.57 (m, 4H), 1.55-1.41 (m, 4H), 1.37-1.26 (m, 7H).

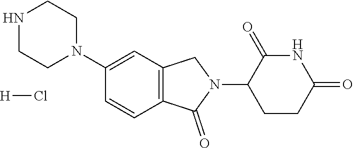

### Recruiter. 3-(1-Oxo-5-(piperazin-1-yl)isoindolin-2-yl)piperidine-2,6-dione hydrochloride

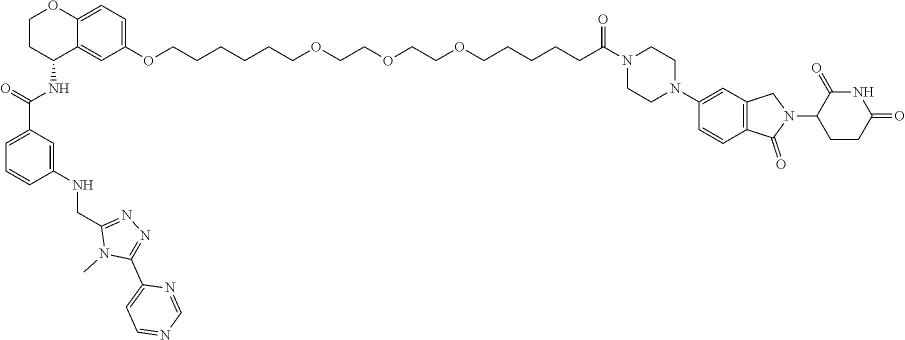 [SMILES: Cn1c(CNc2cccc(C(=O)N[C@@H]3CCOc4ccc(OCCCCCCOCCOCCOCCCCCC(=O)N5CCN(c6ccc7c(c6)CN(C6CCC(=O)NC6=O)C7=O)CC5)cc43)c2)nnc1-c1ccncn1]

Compound 10. D188. N-((4R)-6-(6-(2-(2-(6-(4-(2-(2,6-dioxopiperidin-3-yl)-1-oxoisoindolin-5-yl)piperazin-1-yl)-6-oxohexyloxy)ethoxy)ethoxy)hexyloxy)chroman-4-yl)-3-((4-methyl-5-(pyrimidin-4-yl)-4H-1,2,4-triazol-3-yl)methylamino)benzamide. Colorless oil 86%. LCMS (C18 column 100×4.6 mm, 5.0 μm, pore size 100 Å, water-acetonitrile+0.1% TFA, gradient 5 to 87% for 10 min, retention time 5.87 min). MS (ESI) m/z 1071.2 [MH]+. ¹H NMR (400 MHz, DMSO) δ 10.94 (s, 1H), 9.33 (d, J=1.1 Hz, 1H), 8.97 (d, J=5.3 Hz, 1H), 8.65 (d, J=8.5 Hz, 1H), 8.17 (dd, J=5.3, 1.3 Hz, 1H), 7.53 (d, J=8.4 Hz, 1H), 7.27 (s, 1H), 7.20-7.09 (m, 2H), 7.05 (d, J=8.3 Hz, 2H), 6.90 (d, J=7.6 Hz, 1H), 6.78-6.61 (m, 3H), 6.46 (t, J=5.5 Hz, 1H), 5.22 (d, J=6.9 Hz, 1H), 5.05 (dd, J=13.2, 5.2 Hz, 1H), 4.57 (d, J=5.5 Hz, 2H), 4.33 (d, J=17.1 Hz, 1H), 4.26-4.09 (m, 3H), 4.07 (s, 3H), 3.81 (t, J=6.4 Hz, 2H), 3.62-3.55 (m, 4H), 3.48-3.41 (m, 9H), 3.36-3.22 (m, 7H), 2.89 (dd, J=22.0, 8.8 Hz, 1H), 2.63 (d, J=27.2 Hz, 1H), 2.41-2.27 (m, 3H), 2.09-1.90 (m, 3H), 1.63 (dd, J=20.9, 14.2 Hz, 2H), 1.47 (dt, J=14.6, 7.1 Hz, 6H), 1.36-1.23 (m, 6H).

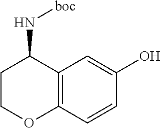

Compound 2. (R)-tert-butyl 6-hydroxychroman-4-ylcarbamate. To an ice cold suspension of amine hydrochloride 1 (1 g, 4.96 mmol) in DCM (20 ml), di-tert-butyl dicarbonate (1.2 g, 5.49 mmol) was added, followed by triethylamine (0.8 ml, 0.58 g, 5.74 mmol). The mixture was stirred at ambient temperature for overnight and quenched with water (10 ml). Organic phase was separated; aqueous phase was extracted with DCM (2 by 10 ml). Combined organic phases were dried over MgSO4 and the solvent was stripped off. The residue was crystallized from DCM-hexane to afford 1.3 g (98%) of compound 2. ¹H NMR (400 MHz, CDCl₃) δ 6.75 (s, 1H), 6.71-6.69 (m, 2H), 5.81 (s, 1H), 4.90 (d, J=8.1 Hz, 1H), 4.82 (s, 1H), 4.21-4.16 (m, 1H), 4.12-4.07 (m, 1H), 2.23-2.05 (m, 1H), 2.05-1.91 (m, 1H), 1.51 (s, 9H).

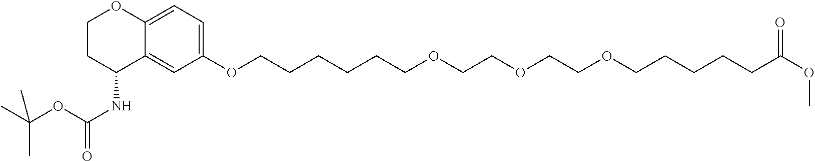 [SMILES: COC(=O)CCCCCOCCOCCOCCCCCCOc1ccc2c(c1)[C@H](NC(=O)OC(C)(C)C)CCO2]

Compound 5. (R)-methyl 6-(2-(2-(6-(4-(tert-butoxycarbonylamino)chroman-6-yloxy)hexyloxy)-ethoxy)ethoxy)hexanoate. Colorless oil 99%. LCMS (C18 column 20×2 mm, 2.5 μm, pore size 100 Å, water-acetonitrile+0.1% TFA, gradient 5 to 87% for 3 min, retention time 2.06 min). MS (ESI) m/z 582.5 [MH]+.

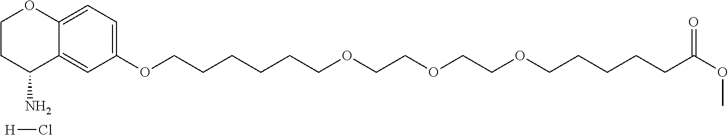

Compound 6. (R)-methyl 6-(2-(2-(6-(4-aminochroman-6-yloxy)hexyloxy)ethoxy)ethoxy)-hexanoate hydrochloride. Colorless crystals 100%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 482.5 [MH]+.

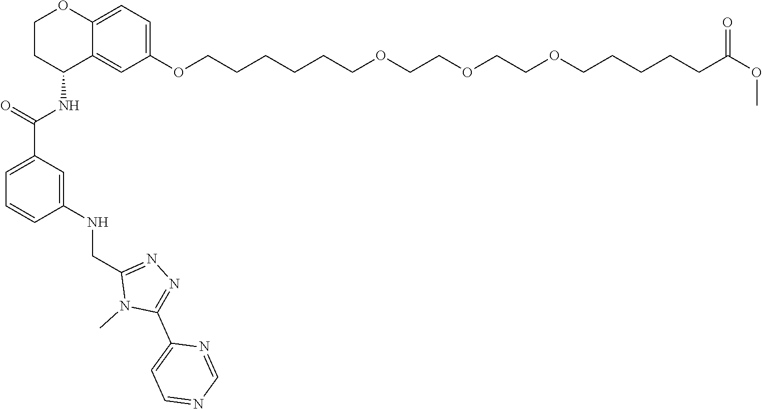 [SMILES: COC(=O)CCCCCOCCOCCOCCCCCCOc1ccc2c(c1)[C@H](NC(=O)c1cccc(NCc3nnc(-c4ccncn4)n3C)c1)CCO2]

Compound 8. (R)-methyl 6-(2-(2-(6-(4-(3-((4-methyl-5-(pyrimidin-4-yl)-4H-1,2,4-triazol-3-yl)methylamino)benzamido)chroman-6-yloxy)hexyloxy)ethoxy)ethoxy)hexanoate. Colorless oil 63%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 774.5 [MH]+. ¹H NMR (400 MHz, CDCl₃) δ 9.29 (d, J=1.2 Hz, 1H), 8.89 (d, J=5.2 Hz, 1H), 8.29 (dd, J=5.3, 1.3 Hz, 1H), 7.31 (s, 1H), 7.29-7.22 (m, 1H), 7.10 (d, J=7.7 Hz, 1H), 7.01-6.90 (m, 1H), 6.79 (s, 3H), 6.49 (d, J=7.3 Hz, 1H), 5.32 (d, J=5.9 Hz, 1H), 4.71 (s, 1H), 4.60 (d, J=5.0 Hz, 2H), 4.26 (d, J=6.0 Hz, 1H), 4.17 (d, J=13.8 Hz, 4H), 3.86 (t, J=6.5 Hz, 2H), 3.75 (t, J=6.6 Hz, 6H), 3.66 (s, 3H), 3.65-3.62 (m, 4H), 3.58-3.57 (m, 4H), 3.46-3.43 (m, 4H), 2.30 (t, J=7.5 Hz, 2H), 2.16 (d, J=5.8 Hz, 1H), 1.90-1.85 (m, 2H), 1.75-1.71 (m, 1H), 1.67-1.58 (m, 4H), 1.45-1.37 (m, 2H).

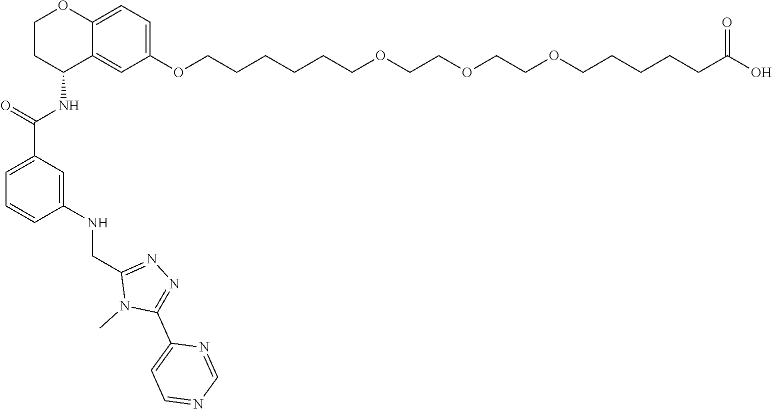 [SMILES: Cn1c(CNc2cccc(C(=O)N[C@@H]3CCOc4ccc(OCCCCCCOCCOCCOCCCCCC(=O)O)cc43)c2)nnc1-c1ccncn1]

Compound 9. (R)-6-(2-(2-(6-(4-(3-((4-methyl-5-(pyrimidin-4-yl)-4H-1,2,4-triazol-3-yl)methyl-amino)benzamido)chroman-6-yloxy)hexyloxy)ethoxy)ethoxy)hexanoic acid. Colorless oil 87%. LCMS (C18 column 20×2 mm, 2.5 μm, pore size 100 Å, water-acetonitrile+0.1% TFA, gradient 5 to 87% for 3 min, retention time 1.43 min). MS (ESI) m/z 760.5 [MH]+.

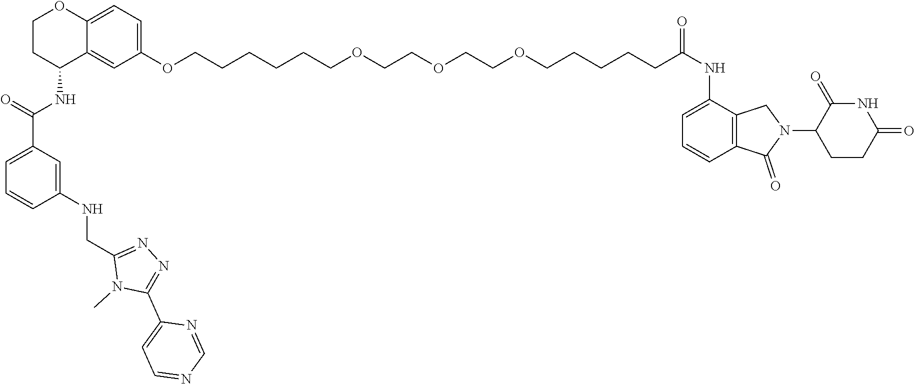 [SMILES: Cn1c(CNc2cccc(C(=O)N[C@@H]3CCOc4ccc(OCCCCCCOCCOCCOCCCCCC(=O)Nc5cccc6c5CN(C5CCC(=O)NC5=O)C6=O)cc43)c2)nnc1-c1ccncn1]

Compound 10. D187. N-((4R)-6-(6-(2-(2-(6-(2-(2,6-dioxopiperidin-3-yl)-1-oxoisoindolin-4-ylamino)-6-oxohexyloxy)ethoxy)ethoxy)hexyloxy)chroman-4-yl)-3-((4-methyl-5-(pyrimidin-4-yl)-4H-1,2,4-triazol-3-yl)methylamino)benzamide. Colorless oil 61%. LCMS (C18 column 100×4.6 mm, 5.0 μm, pore size 100 Å, water-acetonitrile+0.1% TFA, gradient 5 to 87% for 10 min, retention time 5.92 min). MS (ESI) m/z 1002.2 [MH]+. ¹H NMR (400 MHz, DMSO) δ 11.01 (s, 1H), 9.75 (s, 1H), 9.33 (s, 1H), 8.97 (d, J=5.3 Hz, 1H), 8.65 (d, J=8.3 Hz, 1H), 8.17 (d, J=5.3 Hz, 1H), 7.81 (d, J=5.5 Hz, 1H), 7.54-7.43 (m, 2H), 7.26 (s, 1H), 7.20-7.06 (m, 2H), 6.90 (d, J=8.2 Hz, 1H), 6.77-6.62 (m, 3H), 6.46 (t, J=5.5 Hz, 1H), 5.22 (d, J=7.1 Hz, 1H), 5.14 (dd, J=13.4, 5.1 Hz, 1H), 4.57 (d, J=5.6 Hz, 2H), 4.35 (q, J=17.4 Hz, 2H), 4.26-4.20 (m, 1H), 4.13 (t, J=12.7 Hz, 1H), 4.07 (s, 3H), 3.81 (t, J=6.4 Hz, 2H), 3.54-3.39 (m, 8H), 3.38-3.26 (m, 4H), 2.90 (dd, J=21.6, 9.0 Hz, 1H), 2.64 (dd, J=33.1, 13.8 Hz, 1H), 2.40-2.26 (m, 3H), 2.09-1.96 (m, 3H), 1.68-1.56 (m, 4H), 1.55-1.39 (m, 4H), 1.36-1.24 (m, 6H).

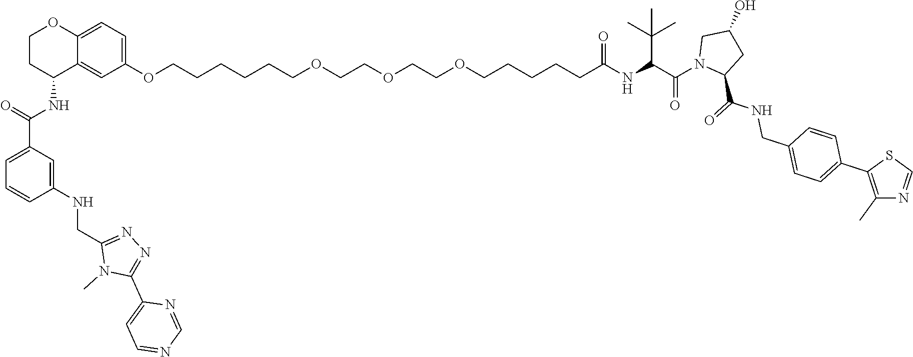

Compound 10. D186. (2S,4R)-1-((R)-2-tert-butyl-22-((R)-4-(3-((4-methyl-5-(pyrimidin-4-yl)-4H-1,2,4-triazol-3-yl)methylamino)benzamido)chroman-6-yloxy)-4-oxo-10,13,16-trioxa-3-azadocosane)-4-hydroxy-N-(4-(4-methylthiazol-5-yl)benzyl)pyrrolidine-2-carboxamide. Colorless oil 26%. LCMS (C18 column 100×4.6 mm, 5.0 μm, pore size 100 Å, water-acetonitrile+0.1% TFA, gradient 5 to 87% for 10 min, retention time 6.36 min). MS (ESI) m/z 1172.9 [MH]+. ¹H NMR (400 MHz, DMSO) δ 9.33 (s, 1H), 8.97 (s, 1H), 8.96 (s, 1H), 8.65 (d, J=8.3 Hz, 1H), 8.55 (t, J=6.0 Hz, 1H), 8.17 (d, J=5.3 Hz, 1H), 7.84 (d, J=9.4 Hz, 1H), 7.39 (q, J=8.2 Hz, 4H), 7.26 (s, 1H), 7.21-7.08 (m, 2H), 6.90 (d, J=7.7 Hz, 1H), 6.80-6.59 (m, 3H), 6.46 (t, J=5.6 Hz, 1H), 5.28-5.15 (m, 1H), 5.11 (d, J=3.5 Hz, 1H), 4.57 (d, J=5.6 Hz, 2H), 4.53 (d, J=9.4 Hz, 1H), 4.43 (dd, J=15.8, 7.2 Hz, 2H), 4.34 (s, 1H), 4.23 (d, J=5.5 Hz, 1H), 4.20-4.11 (m, 1H), 4.07 (s, 3H), 3.81 (t, J=6.4 Hz, 2H), 3.63 (d, J=12.0 Hz, 2H), 3.50-3.46 (m, 4H), 3.43-3.39 (m, 4H), 3.36-3.29 (m, 5H), 2.44 (s, 3H), 2.29-2.21 (m, 1H), 2.13-1.98 (m, 4H), 1.91-1.86 (m, 1H), 1.66-1.57 (m, 2H), 1.52-1.41 (m, 6H), 1.36-1.20 (m, 6H), 0.92 (s, 9H).

### Example 10: Preparation of Compounds D87, D65, D147, and D152

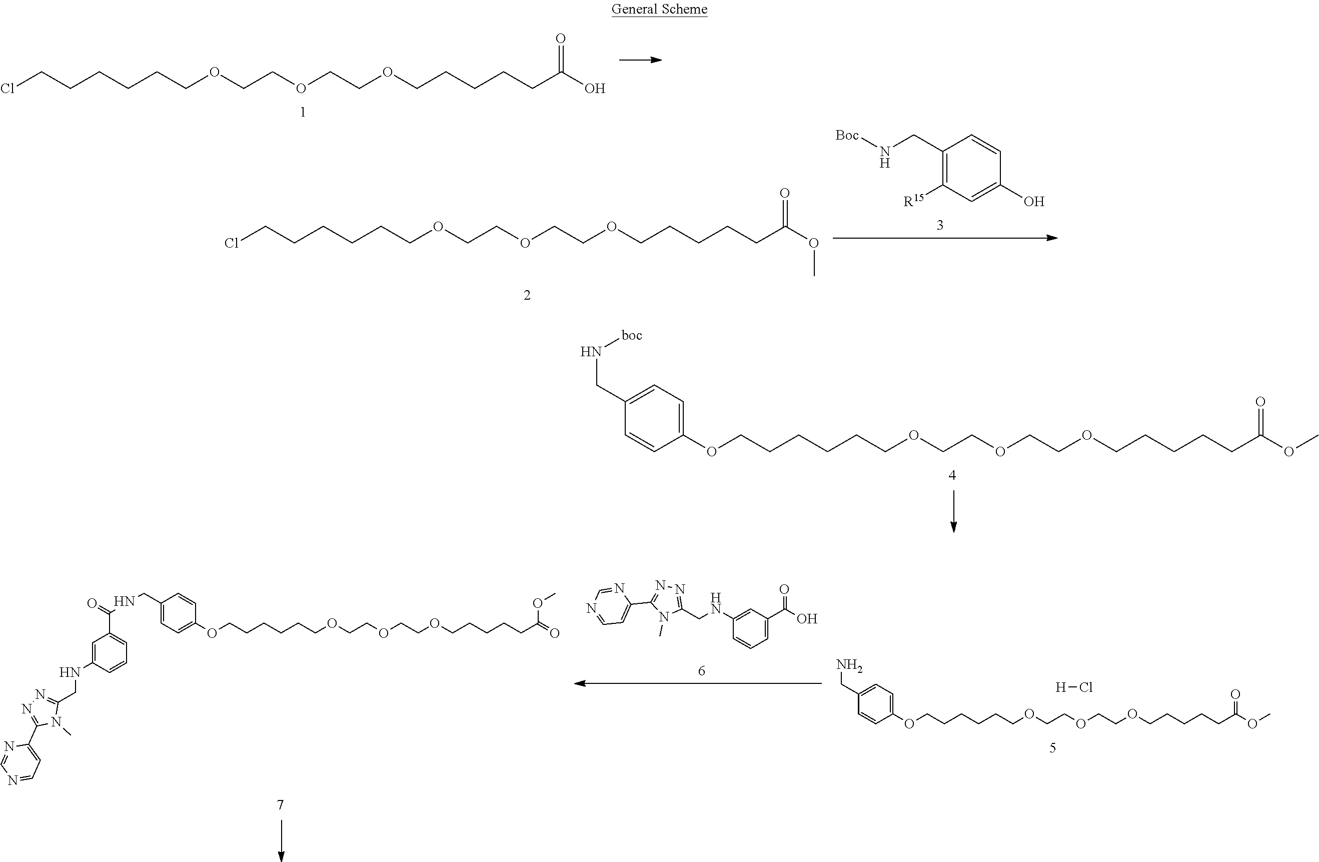

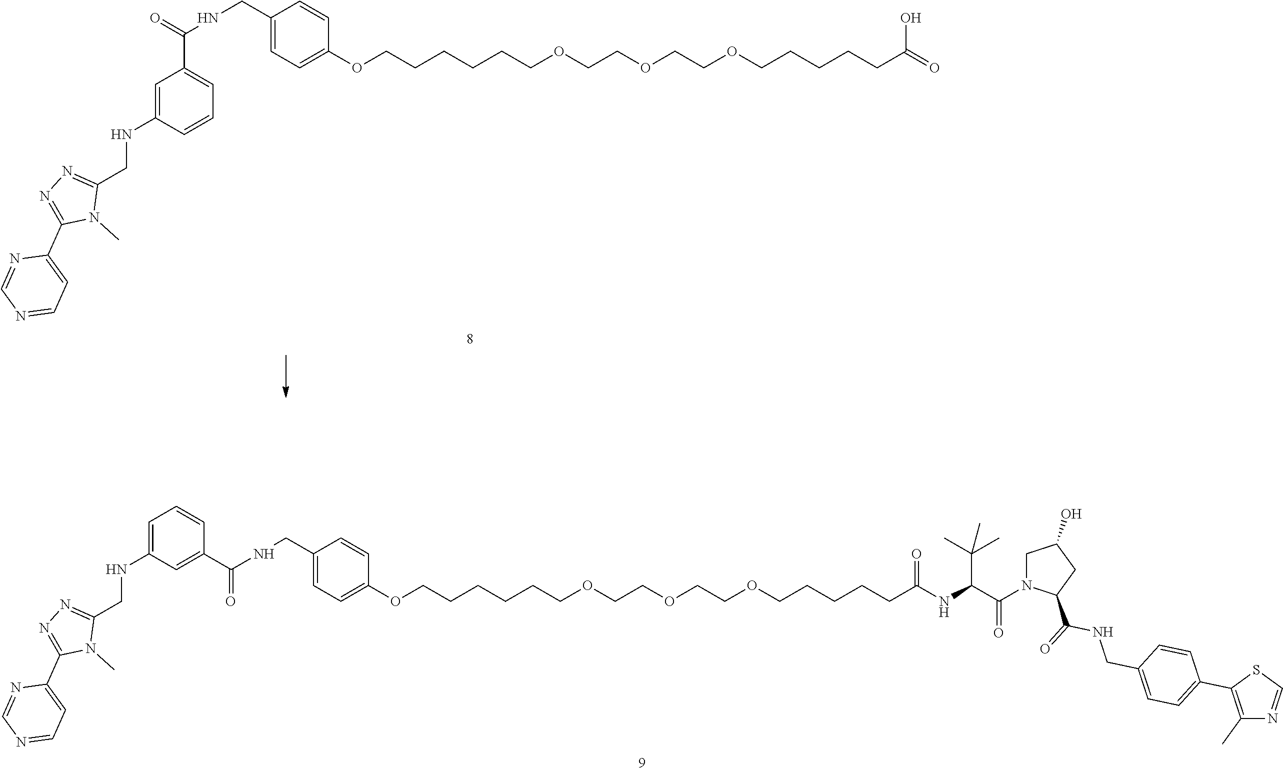 [SMILES: Cn1c(CNc2cccc(C(=O)NCc3ccc(OCCCCCCOCCOCCOCCCCCC(=O)O)cc3)c2)nnc1-c1ccncn1 ; Cc1ncsc1-c1ccc(CNC(=O)[C@@H]2C[C@@H](O)CN2C(=O)[C@@H](NC(=O)CCCCCOCCOCCOCCCCCCOc2ccc(CNC(=O)c3cccc(NCc4nnc(-c5ccncn5)n4C)c3)cc2)C(C)(C)C)cc1]

### Compounds and Analytical Data

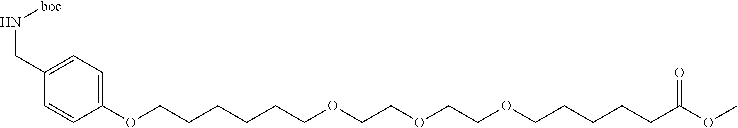

Compound 4. Colorless oil 56%. LCMS (C18 column 20×2 mm, 2.5 μm, pore size 100 Å, water-acetonitrile+0.1% TFA, gradient 5 to 87% for 3 min, retention time 2.07 min). MS (ESI) m/z 540.7 [MH]+.

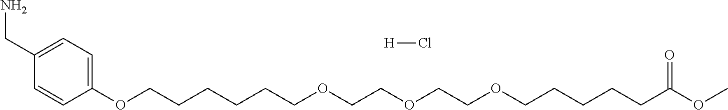

Compound 5. 100%. LCMS (C18 column 20×2 mm, 2.5 μm, pore size 100 Å, water-acetonitrile+0.1% TFA, gradient 5 to 87% for 3 min, retention time 1.27 min). MS (ESI) m/z 477.0 [MH]+.

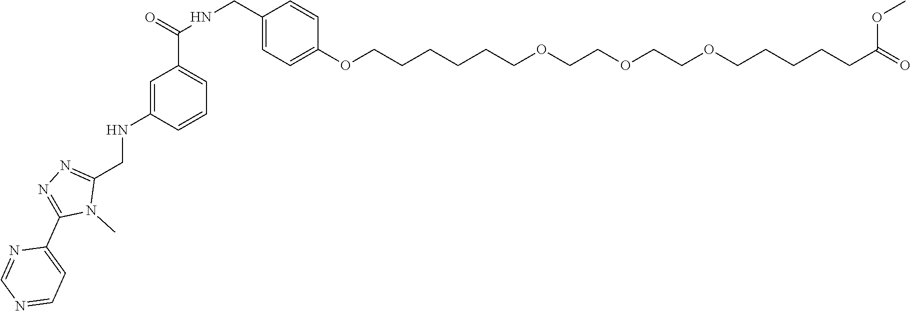 [SMILES: COC(=O)CCCCCOCCOCCOCCCCCCOc1ccc(CNC(=O)c2cccc(NCc3nnc(-c4ccncn4)n3C)c2)cc1]

Compound 7. 34%. LCMS (C18 column 20×2 mm, 2.5 μm, pore size 100-Å, water-acetonitrile+0.1% TFA, gradient 5 to 87% for 3 min, retention time 1.66 min). MS (ESI) m/z 732.9 [MH]+.

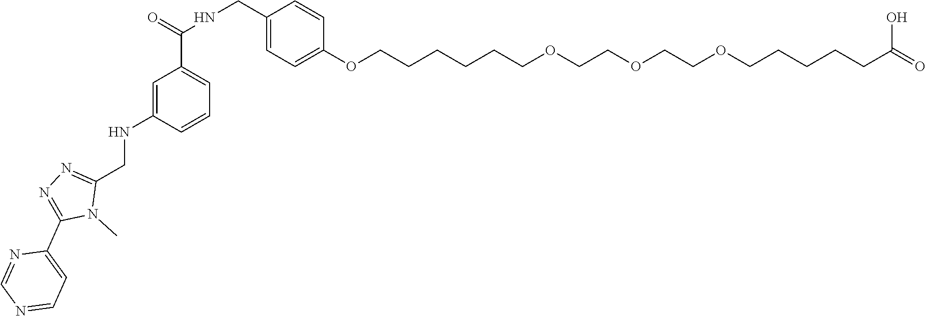 [SMILES: Cn1c(CNc2cccc(C(=O)NCc3ccc(OCCCCCCOCCOCCOCCCCCC(=O)O)cc3)c2)nnc1-c1ccncn1]

Compound 8.47%. LCMS (C18 column 20×2 mm, 2.5 μm, pore size 100-Å, water-acetonitrile+0.1% TFA, gradient 5 to 87% for 3 min, retention time 1.47 min). MS (ESI) m/z 718.8 [MH]+.

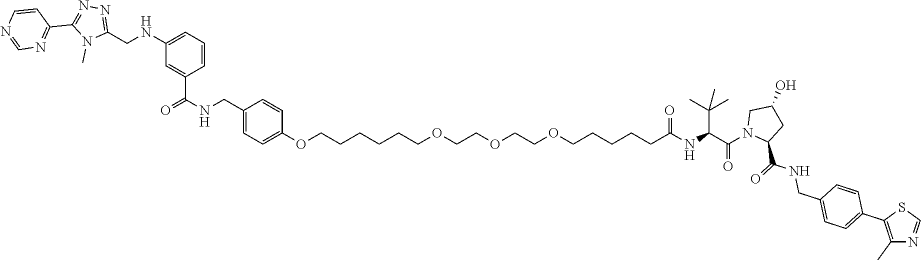 [SMILES: Cc1ncsc1-c1ccc(CNC(=O)[C@@H]2C[C@@H](O)CN2C(=O)[C@@H](NC(=O)CCCCCOCCOCCOCCCCCCOc2ccc(CNC(=O)c3cccc(NCc4nnc(-c5ccncn5)n4C)c3)cc2)C(C)(C)C)cc1]

Compound 9. D87. White powder 13%. LCMS (C18 column 100×4.6 mm, 5.0 μm, pore size 100-Å, water-acetonitrile+0.1% TFA, gradient 5 to 87% for 10 min, retention time 6.43 min). MS (ESI) m/z 1131.4 [MH]+. ¹H NMR (400 MHz, DMSO-d₆) δ 9.33 (s, 1H), 8.97 (d, J=5.7 Hz, 2H), 8.76 (t, J=5.9 Hz, 1H), 8.55 (t, J=5.9 Hz, 1H), 8.17 (d, J=5.3 Hz, 1H), 7.83 (d, J=9.1 Hz, 1H), 7.40 (q, J=8.1 Hz, 4H), 7.25-7.12 (m, 4H), 7.08 (d, J=8.0 Hz, 1H), 6.93-6.81 (m, 3H), 6.45 (t, 1H), 5.11 (d, J=3.5 Hz, 1H), 4.55 (dd, J=12.7, 7.6 Hz, 3H), 4.47-4.39 (m, 2H), 4.36 (d, J=5.6 Hz, 3H), 4.27-4.15 (m, 1H), 4.07 (s, 3H), 3.91 (t, J=6.4 Hz, 2H), 3.71-3.59 (m, 2H), 3.55-3.40 (m, 8H), 3.39-3.32 (m, 4H), 2.44 (s, 3H), 2.31-2.19 (m, 1H), 2.17-1.97 (m, 2H), 1.95-1.85 (m, 1H), 1.76-1.61 (m, 2H), 1.57-1.29 (m, 10H), 1.29-1.17 (m, 2H), 0.93 (s, 9H).

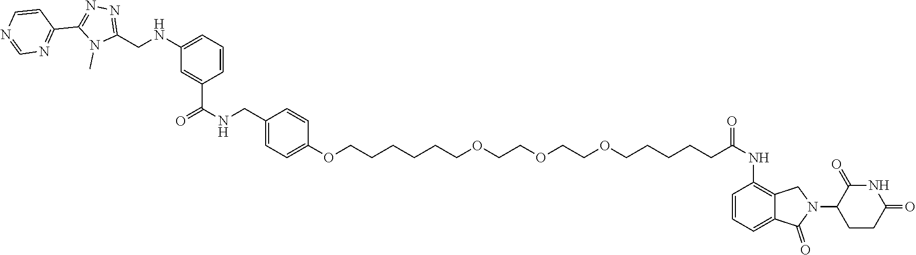 [SMILES: Cn1c(CNc2cccc(C(=O)NCc3ccc(OCCCCCCOCCOCCOCCCCCC(=O)Nc4cccc5c4CN(C4CCC(=O)NC4=O)C5=O)cc3)c2)nnc1-c1ccncn1]

Compound 9. D65. White powder 10%. LCMS (C18 column 100×4.6 mm, 5.0 μm, pore size 100 Å, water-acetonitrile+0.1% TFA, gradient 5 to 87% for 10 min, retention time 6.05 min). MS (ESI) m/z 960.1 [MH]+. ¹H NMR (400 MHz, DMSO-d₆) δ 11.01 (s, 1H), 9.75 (s, 1H), 9.33 (s, 1H), 8.97 (d, J=5.2 Hz, 1H), 8.75 (t, J=6.0 Hz, 1H), 8.17 (d, J=5.3 Hz, 1H), 7.81 (d, J=7.4 Hz, 1H), 7.55-7.43 (m, 2H), 7.27-7.13 (m, 4H), 7.08 (d, J=7.6 Hz, 1H), 6.93-6.80 (m, J=17.0, 9.1 Hz, 3H), 6.46 (t, J=5.7 Hz, 1H), 5.14 (dd, J=13.2, 5.1 Hz, 1H), 4.57 (d, J=5.6 Hz, 2H), 4.43-4.28 (m, 4H), 4.07 (s, 3H), 3.90 (t, J=6.4 Hz, 2H), 3.54-3.41 (m, 8H), 3.36 (dd, J=13.1, 6.6 Hz, 4H), 2.98-2.84 (m, 1H), 2.60 (d, J=14.7 Hz, 1H), 2.35 (t, J=7.5 Hz, 3H), 2.09-1.96 (m, 1H), 1.74-1.56 (m, 4H), 1.56-1.44 (m, 4H), 1.44-1.27 (m, 6H).

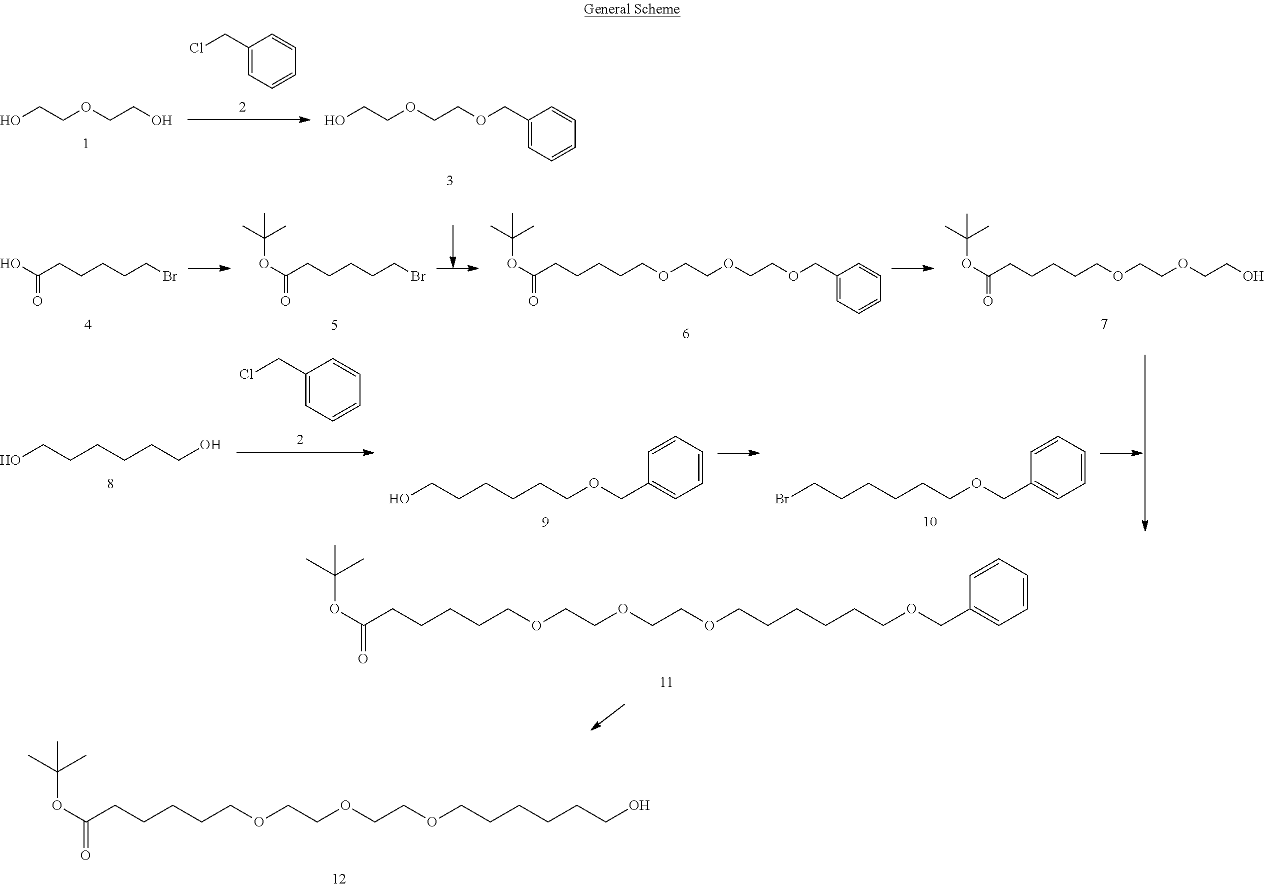

### Compounds, Procedures, and Analytical Data

Compound 3. A mixture of compounds 1 (30 g, 283 mmol, 4 eq), 2 (8.94 g, 79.7 mmol, 0.25 eq.), TBAB (0.2 g) and 50% aqua solution of NaOH (11.3 g, 283 mmol, 1 eq) was stirred at 100° C. for 12 h, then the reaction mass was poured in water (200 ml), the product was extracted with Et2O (3×50 ml), the organic layers were dried over Na₂SO₄, the solvent was removed under reduced pressure. The residue was purified by column chromatography on silica gel, using hexane/EtOAc 1:1 as eluent, to provide the product 3. Yield: 11.5 g (83%). ¹H NMR (400 MHz, DMSO-D₆) δ 7.43-7.21 (m, 5H), 4.62-4.54 (m, 1H), 4.49 (s, 2H), 3.61-3.53 (m, 4H), 3.53-3.47 (m, 2H), 3.47-3.41 (m, 2H), 3.38 (dd, J=13.4, 6.4 Hz, 1H), 1.09 (t, J=7.0 Hz, 1H).

Compound 5. Oxalyl chloride (9.8 g, 76.9 mmol, 1.5 eq.) was added dropwise to a solution of compound 4 (10 g, 51.3 mmol, 1 eq) in CH₂Cl₂ (100 ml) at 10° C., 2 drops of DMF was added too, the reaction mass was stirred at r.t. for 2 h. Then the solvent and excess of oxalyl chloride were removed under reduced pressure, the residue was dissolved in CH₂Cl₂ (100 ml), the mixture was cooled to 10° C., and t-butanol (40 ml) was added to this mixture. The reaction mass was stirred at r.t. for 12 h. Then the mixture was washed with sat. solution of K₂CO₃, the organic layer was dried over Na₂SO₄, the solvent was removed under reduced pressure to provide the product 5. Yield: 12.4 g (96%). ¹H NMR (400 MHz, CDCl₃) δ 3.41 (t, J=6.8 Hz, 2H), 2.23 (t, J=7.4 Hz, 2H), 1.94-1.80 (m, 2H), 1.70-1.56 (m, 2H), 1.53-1.39 (m, 11H).

Compound 6. 35% aqua solution of NaOH (44.5 g, 1.11 mole, 45 eq.) was added to a solution of compound 3 (4.84 g, 24.7 mmol, 1 eq.) and TBAB (2.4 g, 7.41 mmol, 0.3 eq.) in toluene (50 ml). The mixture was stirred at r.t. for 30 min., then compound 5 (12.4 g, 49.0 mmol, 2 eq.) was added, and the reaction mass was stirred at r.t. for 12 h. The mixture was diluted with EtOAc (100 ml), washed with water (3×50 ml), the organic layers were dried over Na₂SO₄, the solvents were removed under reduced pressure. The residue was purified by column chromatography on silica gel, using hexane/Et₂O 5:1 as eluent, to provide the product 6. Yield: 3.74 g (41%). ¹H NMR (400 MHz, DMSO-D₆) δ 7.38-7.24 (m, 5H), 3.56 (s, 2H), 3.49 (ddd, J=8.8, 6.0, 3.6 Hz, 4H), 3.36 (t, J=6.5 Hz, 2H), 2.15 (t, J=7.3 Hz, 2H), 1.55-1.41 (m, 4H), 1.38 (s, 9H), 1.32-1.21 (m, 2H).

Compound 7. H₂ gas was passed through a mixture of compound 6 (3.7 g, 10.1 mmol) and 10% Pd/C (0.9 g) in EtOH (50 ml) at r.t. for 12 h. The powder of Pd/C was filtered, and the solvent was removed under reduced pressure to provide the product 7. Yield: 2.8 g (100%). ¹H NMR (400 MHz, DMSO-D₆) δ 4.56 (t, J=5.5 Hz, 1H), 3.53-3.44 (m, 6H), 3.44-3.38 (m, 2H), 3.38-3.35 (m, 2H), 2.16 (t, J=7.3 Hz, 2H), 1.53-1.43 (m, 4H), 1.39 (s, 9H), 1.33-1.22 (m, 2H).

Compound 9. A mixture of compounds 8 (74.6 g, 632.4 mmol, 4 eq), 2 (20.0 g, 158.1 mmol, 1 eq.), TBAB (0.2 g) and 50% aqua solution of NaOH (25.3 g, 632.4 mmol, 1 eq) was stirred at 100° C. for 12 h, then the reaction mass was poured in water (400 ml), the product was extracted with Et2O (3×100 ml), the organic layers were dried over Na₂SO₄, the solvent was removed under reduced pressure. The residue was purified by column chromatography on silica gel, using hexane/EtOAc 2:1 as eluent, to provide the product 9. Yield: 25.0 g (76%). ¹H NMR (400 MHz, DMSO-D₆) δ 7.40-7.17 (m, 5H), 4.44 (s, 2H), 4.33 (t, J=5.1 Hz, 1H), 3.44-3.35 (m, 4H), 1.57-1.48 (m, 2H), 1.45-1.37 (m, 2H), 1.36-1.22 (m, 4H).

Compound 10. PPh₃ (42.8 g, 163.4 mmol, 1.7 eq.) and CBr₄ (47.9 g, 144.3 mmol, 1.5 eq.) were added to a solution of compound 9 (20 g, 96.1 mmol, 1 eq.) in CH₂Cl₂ (1000 ml) at 0° C., the reaction mass was stirred at r.t. for 12 h. Then Et₂O (1000 ml) was added, the formed precipitate was filtered, the solvents were removed under reduced pressure. The residue was purified by column chromatography on silica gel, using hexane/EtOAc 20:1 as eluent, to provide the product 10. Yield: 23.8 g (91%). ¹H NMR (400 MHz, DMSO-D₆) δ 7.43-7.19 (m, 5H), 4.44 (s, 2H), 3.51 (t, J=6.7 Hz, 2H), 3.41 (t, J=6.5 Hz, 2H), 1.89-1.71 (m, 2H), 1.62-1.47 (m, 2H), 1.45-1.27 (m, 4H).

Compound 11. 35% aqua solution of NaOH (11.7 g, 292.5 mole, 45 eq.) was added to a solution of compound 7 (1.8 g, 6.51 mmol, 1 eq.) and TBAB (0.629 g, 1.95 mmol, 0.3 eq.) in toluene (20 ml). The mixture was stirred at r.t. for 30 min., then compound 10 (2.708 g, 13.02 mmol, 2 eq.) was added, and the reaction mass was stirred at r.t. for 12 h. The mixture was diluted with EtOAc (50 ml), washed with water (3×20 ml), the organic layers were dried over Na₂SO₄, the solvents were removed under reduced pressure. The residue was purified by column chromatography on silica gel, using hexane/Et₂O 3:1, 2:1 as eluents, to provide the product 11. Yield: 1.1 g (36%). ¹H NMR (400 MHz, DMSO-D₆) δ 7.39-7.23 (m, 5H), 4.43 (s, 2H), 3.47 (dq, J=5.9, 3.5 Hz, 8H), 3.41 (t, J=6.5 Hz, 2H), 3.38-3.33 (m, 2H), 2.16 (t, J=7.3 Hz, 2H), 1.57-1.42 (m, 8H), 1.38 (s, 9H), 1.34-1.21 (m, 6H).

Compound 12. H₂ gas was passed through a mixture of compound 11 (1.1 g, 2.36 mmol) and 10% Pd/C (0.3 g) in EtOH (20 ml) at r.t. for 12 h. The powder of Pd/C was filtered, and the solvent was removed under reduced pressure to provide the product 12. Yield: 0.89 g (100%). ¹H NMR (400 MHz, DMSO-d₆) δ 4.30 (t, J=5.1 Hz, 1H), 3.55-3.42 (m, 8H), 3.36 (t, J=6.4 Hz, 6H), 2.17 (t, J=7.3 Hz, 2H), 1.53-1.43 (m, 6H), 1.42-1.36 (m, 11H), 1.33-1.22 (m, 6H).

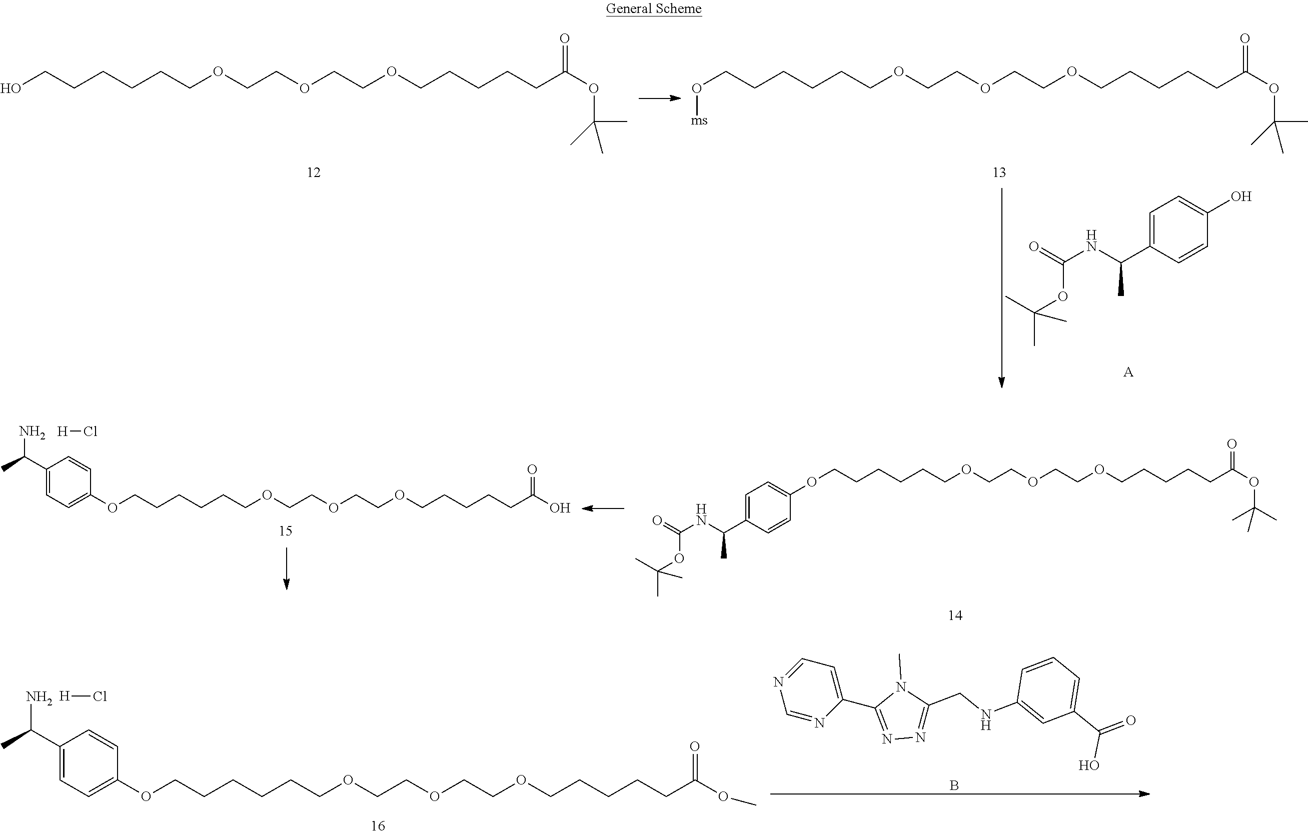

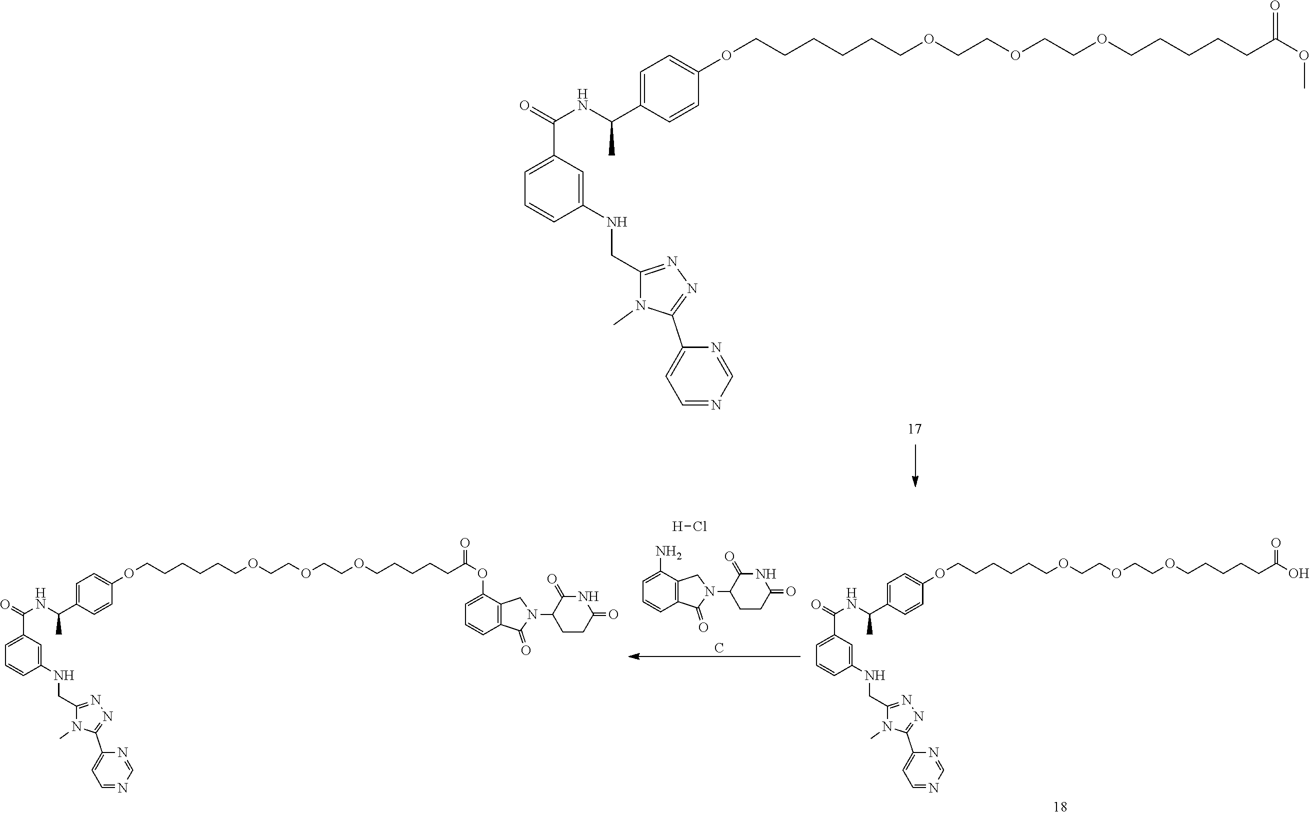

### General Procedures

Compound 13. A solution of compound 12 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 14. A mixture of compound A (2.5 mmol, 1 eq), compound 13 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 14.

Compound 15. A solution of compound 14 (1.2 mmol, 1 eq) in dry DCM (50 mL) with 1.2 ml 3 M HCl in dioxane (5 eq) were stirred for 24 h at r.t. The mixture was evaporated to dryness to give the crude product 15 which was used in the next step without additional purification.

Compound 16. SOCl₂ (0.16 ml, 2.3 mmol, 2 eq) was added dropwise to a stirred solution of compound 15 (1.15 mmol, 1 eq) in dry MeOH (20 mL). The mixture was stirred for 12 h at r.t. and then evaporated to dryness to give the crude product 16 that was used for the next step without additional purification.

Compound 17. A mixture of compound 16 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq), and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 17.

Compound 18. To a solution of compound 17 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D147. A mixture of compound 16 (0.1 mmol, 1 eq), compound C (or other recruiter) (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was purified by HPLC in neutral conditions (YMC-Pack ODS-AQ 250×20 mml, S-10 μm, 12 nm, gradient water-acetonitrile).

### Syntheses According to the General Procedures

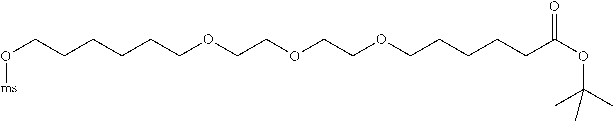

Compound 13. Tetra-butyl-3,10,13,16-tetraoxa-2-thiadocosan-22-oate 2,2-dioxide. Yield 97%

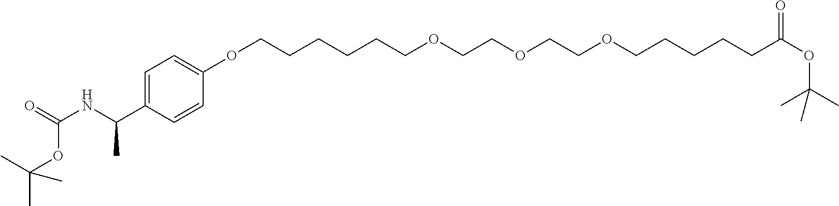 [SMILES: C[C@@H](NC(=O)OC(C)(C)C)c1ccc(OCCCCCCOCCOCCOCCCCCC(=O)OC(C)(C)C)cc1]

Compound 14. (R)-tert-butyl 6-(2-(2-(6-(4-(1-(tert-butoxycarbonylamino)ethyl)phenoxy)hexyloxy) ethoxy)ethoxy)hexanoate. Yield 89%. LCMS (C18 column 20×2 mm, 2.5 μm, pore size 100 Å, water-acetonitrile+0.1% TFA, gradient 5 to 87% for 3 min, retention time 2.37 min). MS (ESI) m/z 596.5 [MH]+. ¹H NMR (400 MHz, DMSO-d₆) δ 7.26 (br, 1H), 7.18 (d, J=8.5 Hz, 2H), 6.84 (d, J=8.5 Hz, 2H), 4.54 (m, 1H), 3.91 (t, J=5.1 Hz, 2H), 3.51-3.44 (m, 8H), 3.39-3.34 (m, 6H), 2.18-2.14 (m, 2H), 1.71-1.63 (m, 2H), 1.54-1.25 (m, 28H).

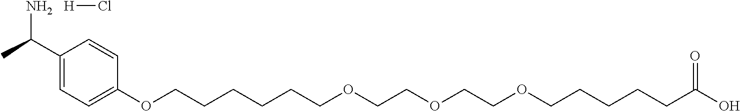

Compound 15. (R)-6-(2-(2-(6-(4-(1-aminoethyl)phenoxy)hexyloxy)ethoxy)ethoxy)hexanoic acid hydrochloride. Yield 99%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 440.5 [MH]+.

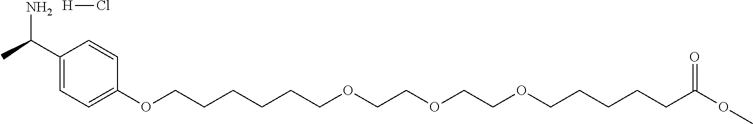

Compound 16. (R)-methyl 6-(2-(2-(6-(4-(1-aminoethyl)phenoxy)hexyloxy)ethoxy)ethoxy)hexanoate hydrochloride. Yield 98%. LCMS (C18 column 20×2 mm, 2.5 μm, pore size 100 Å, water-acetonitrile+0.1% TFA, gradient 5 to 87% for 3 min, retention time 1.27 min). MS (ESI) m/z 454.5 [MH]+.

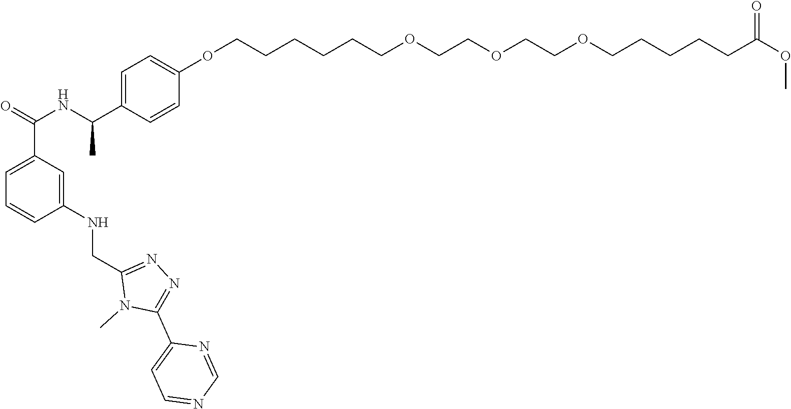 [SMILES: COC(=O)CCCCCOCCOCCOCCCCCCOc1ccc([C@@H](C)NC(=O)c2cccc(NCc3nnc(-c4ccncn4)n3C)c2)cc1]

Compound 17. (R)-methyl 6-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)hexanoate. Yield 68%. LCMS (C18 column 20×2 mm, 2.5 μm, pore size 100 Å, water-acetonitrile+0.1% TFA, gradient 5 to 87% for 3 min, retention time 1.64 min). MS (ESI) m/z 746.7 [MH]+.

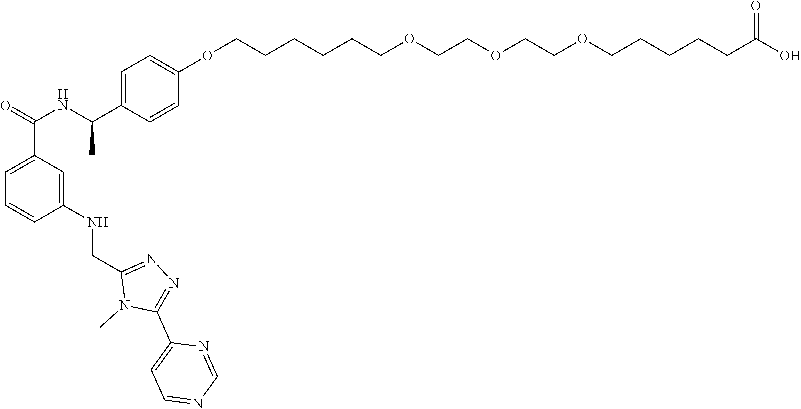 [SMILES: C[C@@H](NC(=O)c1cccc(NCc2nnc(-c3ccncn3)n2C)c1)c1ccc(OCCCCCCOCCOCCOCCCCCC(=O)O)cc1]

Compound 18. (R)-6-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)hexanoic acid. Yield 65%. LCMS (C18 column 20×2 mm, 2.5 μm, pore size 100 Å, water-acetonitrile+0.1% TFA, gradient 5 to 87% for 3 min, retention time 1.22 min). MS (ESI) m/z 732.8[MH]+.

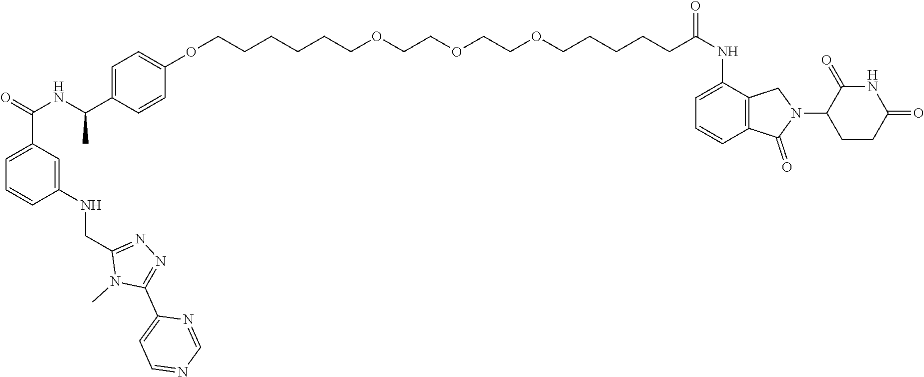 [SMILES: C[C@@H](NC(=O)c1cccc(NCc2nnc(-c3ccncn3)n2C)c1)c1ccc(OCCCCCCOCCOCCOCCCCCC(=O)Nc2cccc3c2CN(C2CCC(=O)NC2=O)C3=O)cc1]

D147. N-((1R)-1-(4-(6-(2-(2-(6-(2-(2,6-dioxopiperidin-3-yl)-1-oxoisoindolin-4-ylamino)-6-oxohexyloxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 35%. LCMS (C18 column 100×4.6 mm, 5.0 μm, pore size 100 Å, water-acetonitrile+0.1% TFA, gradient 5 to 87% for 10 min, retention time 6.12 min). MS (ESI) m/z 974.3 [MH]+. ¹H NMR (400 MHz, DMSO-d₆) δ 11.01 (s, 1H), 9.75 (s, 1H), 9.33 (s, 1H), 8.97 (d, J=5.3 Hz, 1H), 8.50 (d, J=8.1 Hz, 1H), 8.18 (dd, J=5.3, 1.3 Hz, 1H), 7.80 (dd, J=7.1, 1.5 Hz, 1H), 7.51-7.45 (m, 2H), 7.26-7.17 (m, 4H), 7.07 (d, J=5.7 Hz, 1H), 6.90-6.83 (m, 3H), 6.44 (t, J=5.7 Hz, 1H), 5.16-5.06 (m, 2H), 4.56 (d, J=17.6 Hz, 2H), 4.41-4.30 (m, 2H), 4.07 (s, 3H), 3.90 (t, J=6.5 Hz, 2H), 3.52-3.43 (m, 8H), 3.39-3.34 (m, 2H), 2.96-2.89 (m, 1H), 2.67-2.54 (m, 2H), 2.38-2.29 (m, 3H), 2.05-1.99 (m, 1H), 1.69-1.57 (m, 4H), 1.53-1.30 (m, 14H).

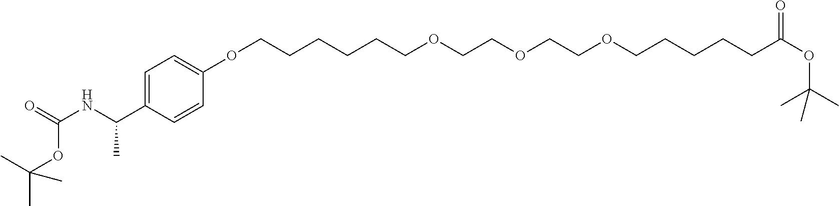 [SMILES: C[C@H](NC(=O)OC(C)(C)C)c1ccc(OCCCCCCOCCOCCOCCCCCC(=O)OC(C)(C)C)cc1]

Compound 14. (S)-tert-butyl 6-(2-(2-(6-(4-(1-(tert-butoxycarbonylamino)ethyl)phenoxy)hexyloxy) ethoxy)ethoxy)hexanoate. Yield 89%. LCMS (C18 column 20×2 mm, 2.5 μm, pore size 100 Å, water-acetonitrile+0.1% TFA, gradient 5 to 87% for 3 min, retention time 2.37 min). MS (ESI) m/z 596.5 [MH]+. ¹H NMR (400 MHz, DMSO) δ 7.26 (br, 1H), 7.18 (d, J=8.5 Hz, 2H), 6.84 (d, J=8.5 Hz, 2H), 4.54 (m, 1H), 3.91 (t, J=5.1 Hz, 2H), 3.51-3.44 (m, 8H), 3.39-3.34 (m, 6H), 2.18-2.14 (m, 2H), 1.71-1.63 (m, 2H), 1.54-1.25 (m, 28H).

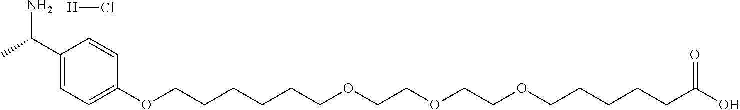

Compound 15. (S)-6-(2-(2-(6-(4-(1-aminoethyl)phenoxy)hexyloxy)ethoxy)ethoxy)hexanoic acid hydrochloride. Yield 99%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 440.5 [MH]+.

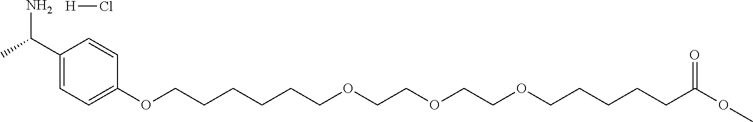

Compound 16. (S)-methyl 6-(2-(2-(6-(4-(1-aminoethyl)phenoxy)hexyloxy)ethoxy)ethoxy)hexanoate hydrochloride. Yield 98%. LCMS (C18 column 20×2 mm, 2.5 μm, pore size 100 Å, water-acetonitrile+0.1% TFA, gradient 5 to 87% for 3 min, retention time 1.27 min). MS (ESI) m/z 454.5 [MH]+.

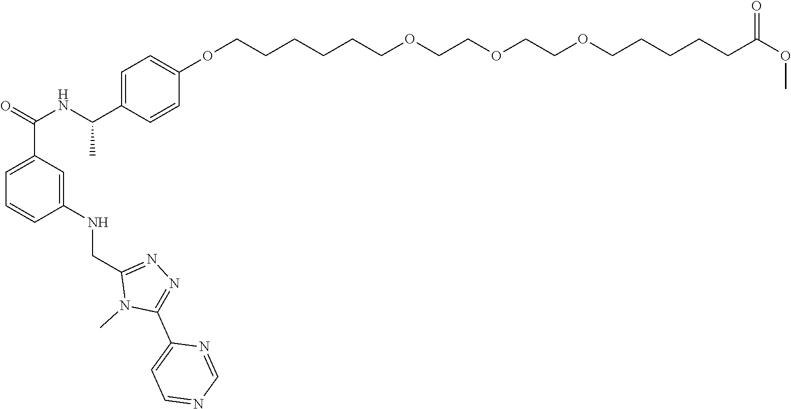 [SMILES: COC(=O)CCCCCOCCOCCOCCCCCCOc1ccc([C@H](C)NC(=O)c2cccc(NCc3nnc(-c4ccncn4)n3C)c2)cc1]

Compound 17. (S)-methyl 6-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)hexanoate. Yield 68%. LCMS (C18 column 20×2 mm, 2.5 μm, pore size 100 Å, water-acetonitrile+0.1% TFA, gradient 5 to 87% for 3 min, retention time 1.64 min). MS (ESI) m/z 746.7 [MH]+.

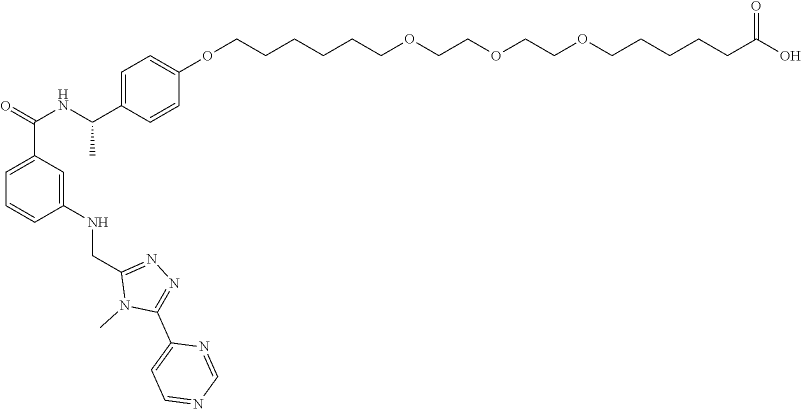 [SMILES: C[C@H](NC(=O)c1cccc(NCc2nnc(-c3ccncn3)n2C)c1)c1ccc(OCCCCCCOCCOCCOCCCCCC(=O)O)cc1]

Compound 18. (S)-6-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)hexanoic acid. Yield 65%. LCMS (C18 column 20×2 mm, 2.5 μm, pore size 100 Å, water-acetonitrile+0.1% TFA, gradient 5 to 87% for 3 min, retention time 1.22 min). MS (ESI) m/z 732.8[MH]+.

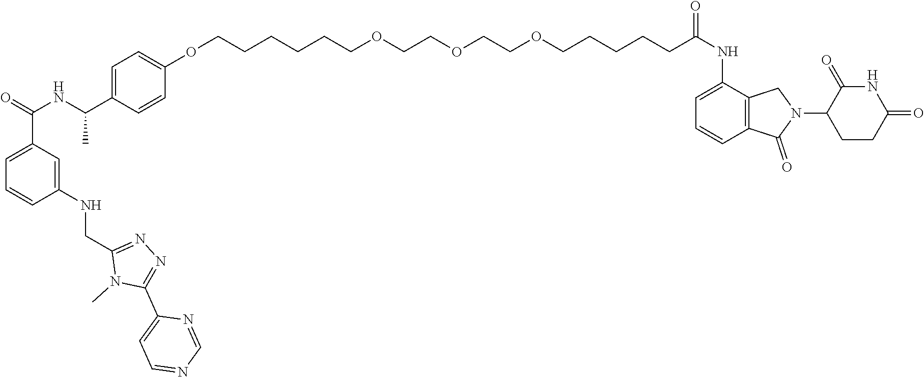 [SMILES: C[C@H](NC(=O)c1cccc(NCc2nnc(-c3ccncn3)n2C)c1)c1ccc(OCCCCCCOCCOCCOCCCCCC(=O)Nc2cccc3c2CN(C2CCC(=O)NC2=O)C3=O)cc1]

D152. N-((1S)-1-(4-(6-(2-(2-(6-(2-(2,6-dioxopiperidin-3-yl)-1-oxoisoindolin-4-ylamino)-6-oxohexyloxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 31%. LCMS (C18 column 100×4.6 mm, 5.0 μm, pore size 100 Å, water-acetonitrile+0.1% TFA, gradient 5 to 87% for 10 min, retention time 6.12 min). MS (ESI) m/z 974.3 [MH]+. ¹H NMR (400 MHz, DMSO-d₆) δ 11.01 (s, 1H), 9.75 (s, 1H), 9.33 (s, 1H), 8.97 (d, J=5.3 Hz, 1H), 8.50 (d, J=8.1 Hz, 1H), 8.18 (dd, J=5.3, 1.3 Hz, 1H), 7.80 (dd, J=7.1, 1.5 Hz, 1H), 7.51-7.45 (m, 2H), 7.26-7.17 (m, 4H), 7.07 (d, J=5.7 Hz, 1H), 6.90-6.83 (m, 3H), 6.44 (t, J=5.7 Hz, 1H), 5.16-5.06 (m, 2H), 4.56 (d, J=17.6 Hz, 2H), 4.41-4.30 (m, 2H), 4.07 (s, 3H), 3.90 (t, J=6.5 Hz, 2H), 3.52-3.43 (m, 8H), 3.39-3.34 (m, 4H), 2.96-2.89 (m, 1H), 2.67-2.54 (m, 1H), 2.38-2.29 (m, 3H), 2.05-1.99 (m, 1H), 1.69-1.57 (m, 4H), 1.53-1.30 (m, 13H).

### Example 11: Preparation of Compounds D112 and D110

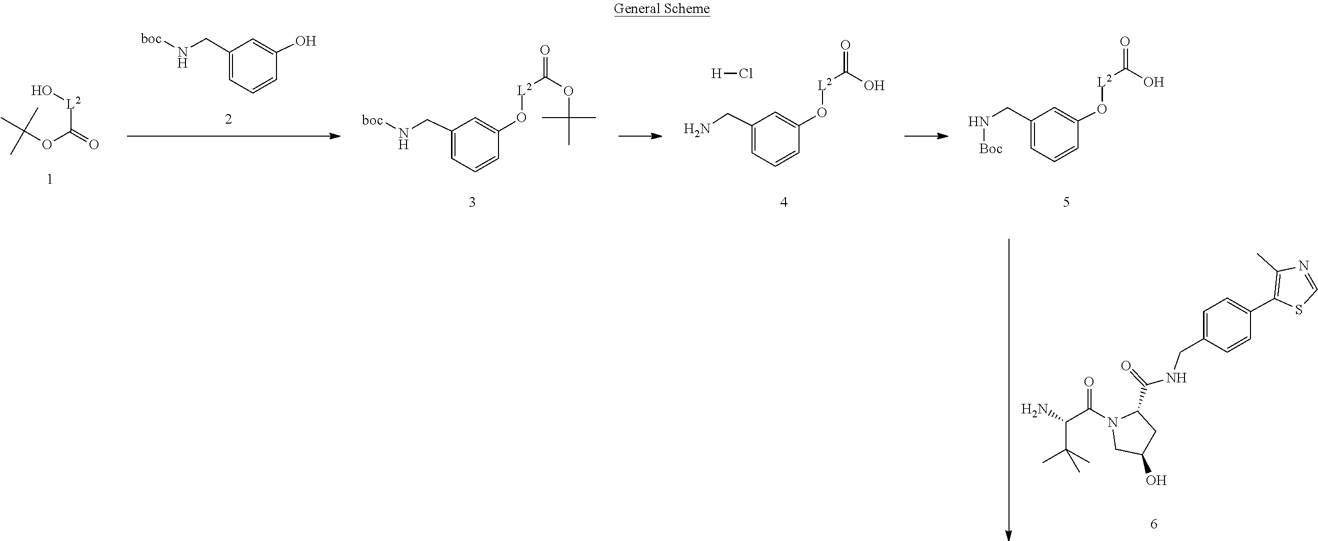

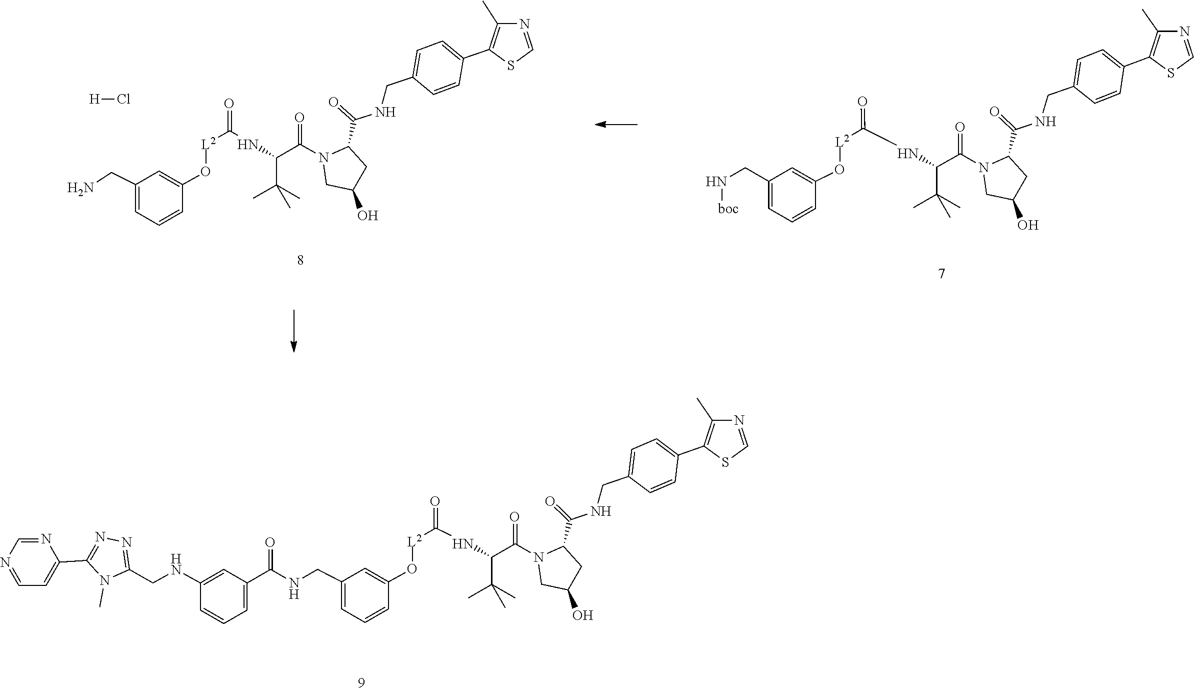

### Compounds, Procedures, and Analytical Data

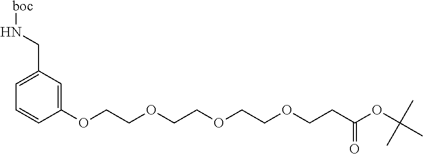

Compound 3. A solution of compound 1 (6.1 mmol, 1 eq), compound 2 (1.71 g, 6.1 mmol, 1 eq) and triphenilphosphine (1.61 g, 6.1 mmol, 1 eq) in dry THF (50 mL) under argon was cooled to 0° C., then DIAD (1.24 g, 6.1 mmol, 1 eq) was added. The mixture was stirred at r.t. for 16 h. THF was removed under reduced pressure, the residue was purified by column chromatography eluting with hexane-EtOAc—NH₄OH 66:33:5. Light yellow oil 94%. LCMS (C18 column 20×2 mm, 2.5 μm, pore size 100 Å, water-acetonitrile+0.1% TFA, gradient 5 to 87% for 3 min, retention time 1.71 min). MS (ESI) m/z 484.6 [MH]+.

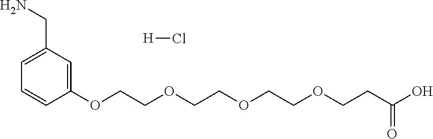 [SMILES: NCc1cccc(OCCOCCOCCOCCC(=O)O)c1 ; Cl]

Compound 4. 100% crude. LCMS (C18 column 20×2 mm, 2.5 μm, pore size 100 Å, water-acetonitrile+0.1% TFA, gradient 5 to 87% for 3 min, retention time 1.57 min). MS (ESI) m/z 328.4 [MH]+.

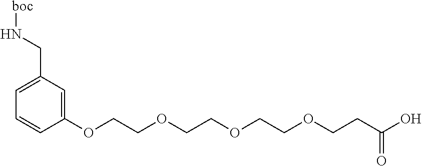

Compound 5. Light yellow oil 48%. LCMS (C18 column 20×2 mm, 2.5 μm, pore size 100 Å, water-acetonitrile+0.1% TFA, gradient 5 to 87% for 3 min, retention time 1.29 min). MS (ESI) m/z 428. ¹H NMR. 4 [MH]+ (400 MHz, DMSO-d₆) δ 12.14 (s, 1H), 7.34 (t, J=7.5 Hz, 1H), 7.21 (t, J=7.9 Hz, 1H), 6.87-6.69 (m, 3H), 4.15-3.98 (m, 4H), 3.79-3.67 (m, 2H), 3.65-3.44 (m, 10H), 2.43 (t, J=6.3 Hz, 2H), 1.36 (s, 9H).

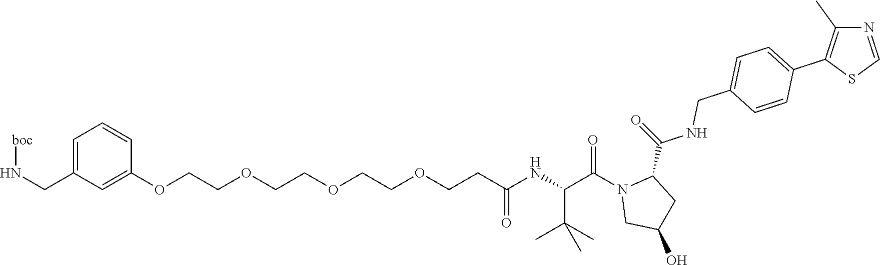

Compound 7. 54%. LCMS (C18 column 20×2 mm, 2.5 μm, pore size 100 Å, water-acetonitrile+0.1% TFA, gradient 5 to 87% for 3 min, retention time 1.50 min). MS (ESI) m/z 841.0 [MH]+.

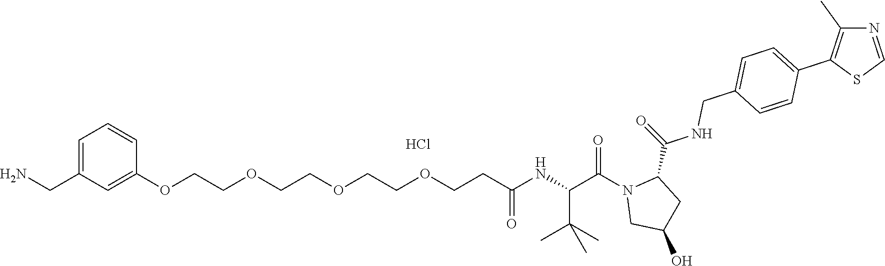 [SMILES: Cc1ncsc1-c1ccc(CNC(=O)[C@@H]2C[C@@H](O)CN2C(=O)[C@@H](NC(=O)CCOCCOCCOCCOc2cccc(CN)c2)C(C)(C)C)cc1 ; Cl]

Compound 8. 100% crude. LCMS (C18 column 20×2 mm, 2.5 μm, pore size 100 Å, water-acetonitrile+0.1% TFA, gradient 5 to 87% for 3 min, retention time 1.11 min). MS (ESI) m/z 741.0 [MH]+.

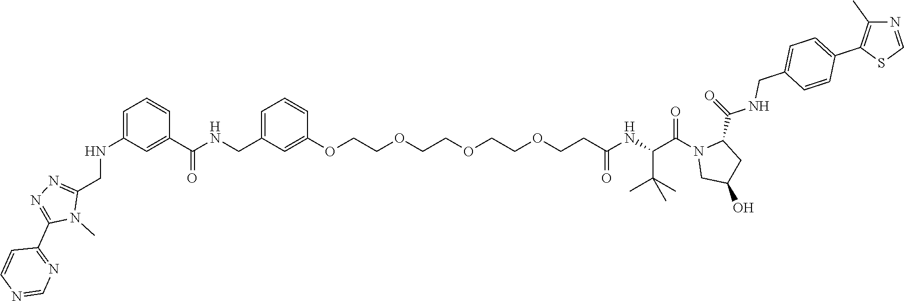 [SMILES: Cc1ncsc1-c1ccc(CNC(=O)[C@@H]2C[C@@H](O)CN2C(=O)[C@@H](NC(=O)CCOCCOCCOCCOc2cccc(CNC(=O)c3cccc(NCc4nnc(-c5ccncn5)n4C)c3)c2)C(C)(C)C)cc1]

Compound 9. D112. White powder 31%. LCMS (C18 column 100×4.6 mm, 5.0 μm, pore size 100 Å, water-acetonitrile+0.1% TFA, gradient 5 to 87% for 10 min, retention time 5.42 min). MS (ESI) m/z 1033.2 [MH]+. ¹H NMR (400 MHz, DMSO-d₆) δ 9.33 (s, 1H), 9.02-8.92 (m, 2H), 8.81 (t, J=7.2 Hz, 1H), 8.55 (t, J=7.5 Hz, 1H), 8.17 (d, J=5.2 Hz, 1H), 7.89 (d, J=9.4 Hz, 1H), 7.39 (q, J=8.2 Hz, 4H), 7.28-7.14 (m, 3H), 7.10 (d, J=8.1 Hz, 1H), 6.93-6.83 (m, 3H), 6.80 (d, J=8.3 Hz, 1H), 6.46 (t, J=7.0 Hz, 1H), 5.12 (d, J=3.5 Hz, 1H), 4.62-4.51 (m, 3H), 4.47-4.38 (m, 4H), 4.35 (s, 1H), 4.26-4.17 (m, 1H), 4.11-3.99 (m, 5H), 3.77-3.42 (m, 15H), 2.44 (s, 3H), 2.39-2.29 (m, 1H), 2.09-1.97 (m, 1H), 1.96-1.84 (m, 1H), 0.93 (s, 9H).

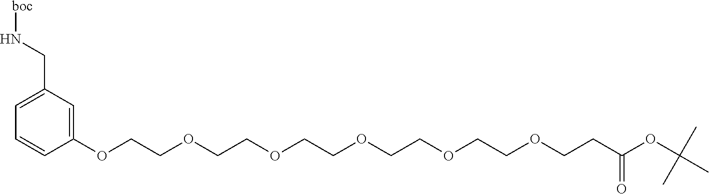

Compound 3. A solution of compound 1 (4.5 mmol, 1 eq), compound 2 (1.01 g, 4.5 mmol, 1 eq) and triphenilphosphine (1.19 g, 4.5 mmol, 1 eq) in dry THF (50 mL) under argon was cooled to 0° C., then diisopropyl azodicarboxylate (DIAD) (0.92 g, 4.5 mmol, 1 eq) was added. The mixture was stirred at r. t. for 16 h. THF was removed under reduced pressure, the residue was purified by column chromatography eluting with hexane-EtOAc—NH₄OH 66:33:5. Light yellow oil 77%. LCMS (C18 column 20×2 mm, 2.5 μm, pore size 100 Å, water-acetonitrile+0.1% TFA, gradient 5 to 87% for 3 min, retention time 1.64 min). MS (ESI) m/z 572.7 [MH]+.

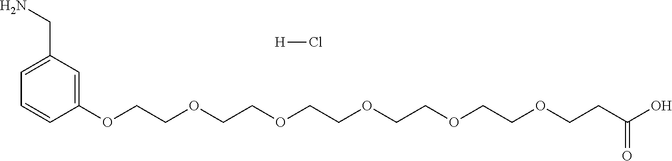 [SMILES: NCc1cccc(OCCOCCOCCOCCOCCOCCC(=O)O)c1 ; Cl]

Compound 4. 100% crude. LCMS (C18 column 20×2 mm, 2.5 μm, pore size 100 Å, water-acetonitrile+0.1% TFA, gradient 5 to 87% for 3 min, retention time 1.12 min). MS (ESI) m/z 416.5 [MH]+.

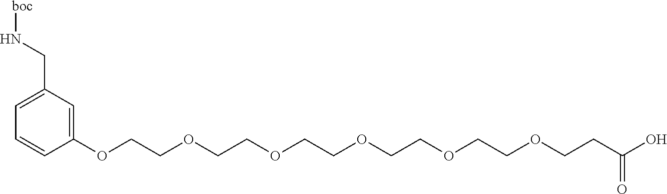

Compound 5. Light yellow oil 48%. LCMS (C18 column 20×2 mm, 2.5 μm, pore size 100 Å, water-acetonitrile+0.1% TFA, gradient 5 to 87% for 3 min, retention time 1.32 min). MS (ESI) m/z 516.5 [MH]+.

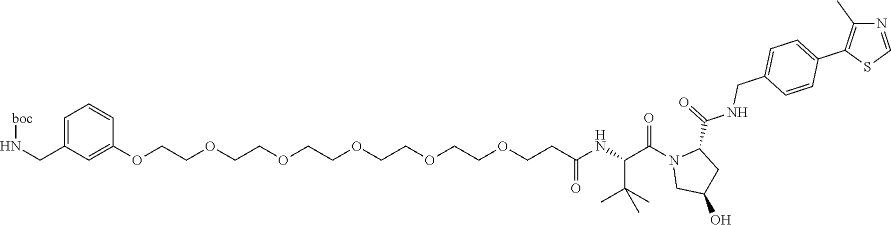

Compound 7. 40%. LCMS (C18 column 20×2 mm, 2.5 μm, pore size 100 Å, water-acetonitrile+0.1% TFA, gradient 5 to 87% for 3 min, retention time 1.87 min). MS (ESI) m/z 929.0 [MH]+.

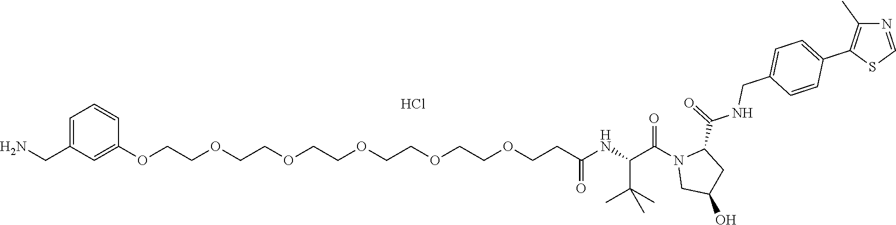 [SMILES: Cc1ncsc1-c1ccc(CNC(=O)[C@@H]2C[C@@H](O)CN2C(=O)[C@@H](NC(=O)CCOCCOCCOCCOCCOCCOc2cccc(CN)c2)C(C)(C)C)cc1 ; Cl]

Compound 8. 40%. LCMS (C18 column 20×2 mm, 2.5 μm, pore size 100 Å, water-acetonitrile+0.1% TFA, gradient 5 to 87% for 3 min, retention time 1.10 min). MS (ESI) m/z 829.0 [MH]+.

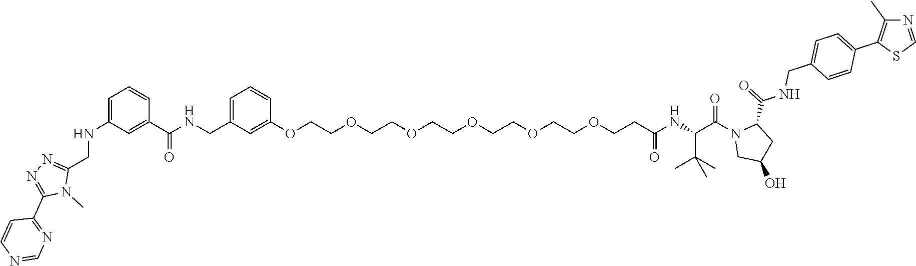 [SMILES: Cc1ncsc1-c1ccc(CNC(=O)[C@@H]2C[C@@H](O)CN2C(=O)[C@@H](NC(=O)CCOCCOCCOCCOCCOCCOc2cccc(CNC(=O)c3cccc(NCc4nnc(-c5ccncn5)n4C)c3)c2)C(C)(C)C)cc1]

Compound 9. D110. White powder 31%. LCMS (C18 column 100×4.6 mm, 5.0 μm, pore size 100 Å, water-acetonitrile+0.1% TFA, gradient 5 to 87% for 10 min, retention time 5.35 min). MS (ESI) m/z 1121.3 [MH]+. ¹H NMR (400 MHz, DMSO-d₆) δ 9.33 (s, 1H), 9.04-8.91 (m, 2H), 8.81 (t, J=5.2 Hz, 1H), 8.55 (t, J=5.1 Hz, 1H), 8.18 (d, J=5.0 Hz, 1H), 7.90 (d, J=9.1 Hz, 1H), 7.40 (q, J=8.4 Hz, 4H), 7.28-7.14 (m, 3H), 7.10 (d, J=7.3 Hz, 1H), 6.97-6.83 (m, 3H), 6.80 (d, J=8.6 Hz, 1H), 6.47 (t, J=5.0 Hz, 1H), 5.11 (s, 1H), 4.63-4.51 (m, 3H), 4.41 (d, J=6.0 Hz, 4H), 4.35 (s, 1H), 4.21 (d, J=11.4 Hz, 1H), 4.12-4.00 (m, 5H), 3.77-3.41 (m, 23H), 2.44 (s, 3H), 2.40-2.27 (m, 1H), 2.09-1.97 (m, 1H), 1.97-1.82 (m, 1H), 0.93 (s, 9H).

### Example 12: Preparation of Compounds D111 and D107

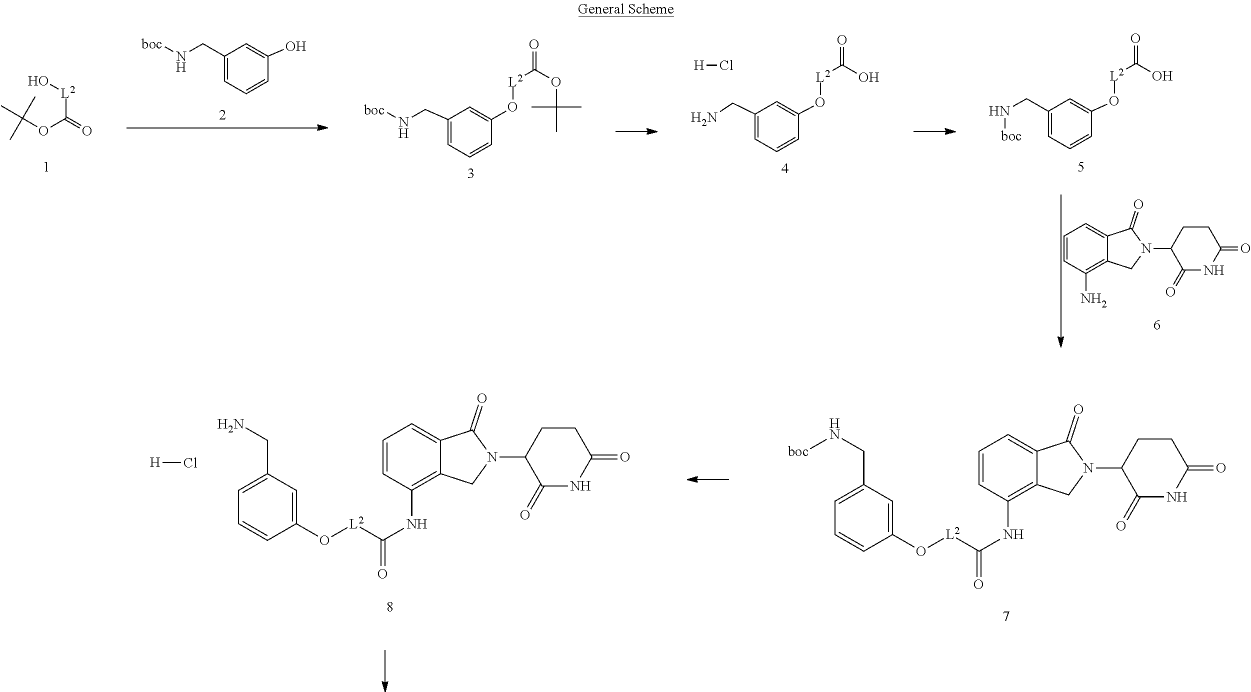

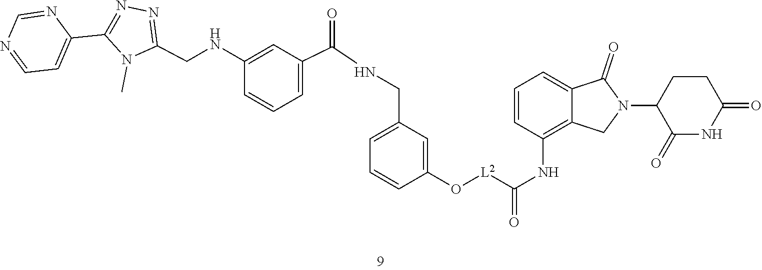

### Compounds, Procedures, and Analytical Data

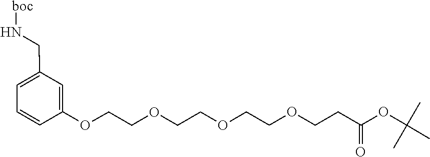

Compound 3. A solution of compound 1 (6.1 mmol, 1 eq), compound 2 (1.71 g, 6.1 mmol, 1 eq) and triphenilphosphine (1.61 g, 6.1 mmol, 1 eq) in dry THF (50 mL) under argon was cooled to 0° C., then DIAD (1.24 g, 6.1 mmol, 1 eq) was added. The mixture was stirred at rt for 16 h. THF was removed under reduced pressure, the residue was purified by column chromatography eluting with hexane-EtOAc—NH4OH 66:33:5. Light yellow oil 94%. LCMS (C18 column 20×2 mm, 2.5 μm, pore size 100 Å, water-acetonitrile+0.1% TFA, gradient 5 to 87% for 3 min, retention time 1.71 min). MS (ESI) m/z 484.6 [MH]+.

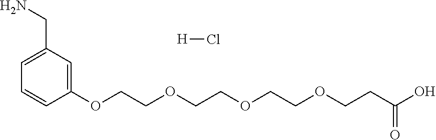 [SMILES: NCc1cccc(OCCOCCOCCOCCC(=O)O)c1 ; Cl]

Compound 4. 100% crude. LCMS (C18 column 20×2 mm, 2.5 μm, pore size 100 Å, water-acetonitrile+0.1% TFA, gradient 5 to 87% for 3 min, retention time 1.57 min). MS (ESI) m/z 328.4 [MH]+.

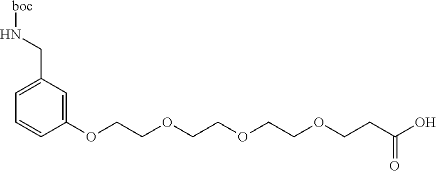

Compound 5. Light yellow oil 48%. LCMS (C18 column 20×2 mm, 2.5 μm, pore size 100 Å, water-acetonitrile+0.1% TFA, gradient 5 to 87% for 3 min, retention time 1.29 min). MS (ESI) m/z 428.4 [MH]+. ¹H NMR (400 MHz, DMSO-d₆) δ 12.14 (s, 1H), 7.34 (t, J=7.5 Hz, 1H), 7.21 (t, J=7.9 Hz, 1H), 6.87-6.69 (m, 3H), 4.15-3.98 (m, 4H), 3.79-3.67 (m, 2H), 3.65-3.44 (m, 10H), 2.43 (t, J=6.3 Hz, 2H), 1.36 (s, 9H).

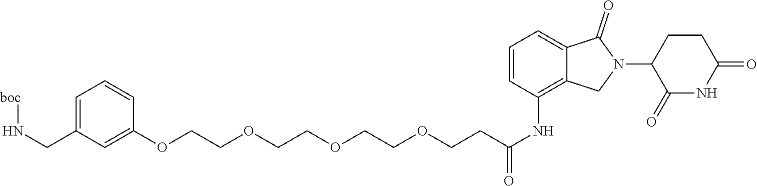

Compound 7. Light yellow oil 88%. LCMS (C18 column 20×2 mm, 2.5 μm, pore size 100 Å, water-acetonitrile+0.1% TFA, gradient 5 to 87% for 3 min, retention time 1.41 min). MS (ESI) m/z 669.7 [MH]+.

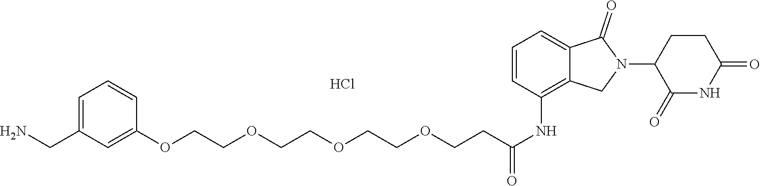 [SMILES: NCc1cccc(OCCOCCOCCOCCC(=O)Nc2cccc3c2CN(C2CCC(=O)NC2=O)C3=O)c1 ; Cl]

Compound 8. 43% crude. LCMS (C18 column 20×2 mm, 2.5 μm, pore size 100 Å, water-acetonitrile+0.1% TFA, gradient 5 to 87% for 3 min, retention time 0.99 min). MS (ESI) m/z 569.7 [MH]+.

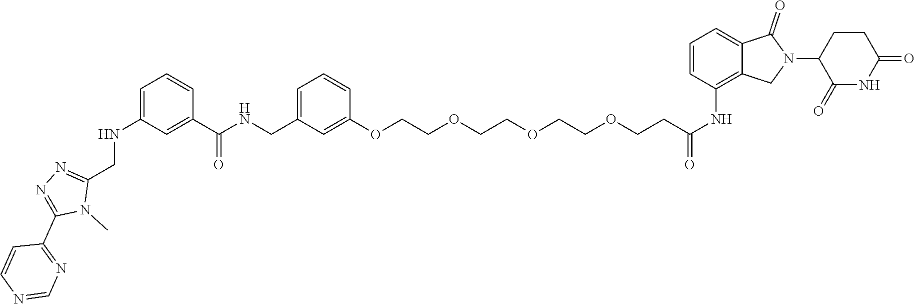 [SMILES: Cn1c(CNc2cccc(C(=O)NCc3cccc(OCCOCCOCCOCCC(=O)Nc4cccc5c4CN(C4CCC(=O)NC4=O)C5=O)c3)c2)nnc1-c1ccncn1]

Compound 9. D111. Yellow powder 42%. LCMS (C18 column 100×4.6 mm, 5.0 μm, pore size 100 Å, water-acetonitrile+0.1% TFA, gradient 5 to 87% for 10 min, retention time 4.97 min). MS (ESI) m/z 861.9 [MH]+. ¹H NMR (400 MHz, DMSO-d₆) δ 11.01 (s, 1H), 9.83 (s, 1H), 9.33 (s, 1H), 8.97 (d, J=5.2 Hz, 1H), 8.82 (t, J=5.6 Hz, 1H), 8.17 (d, J=4.8 Hz, 1H), 7.81 (d, J=7.0 Hz, 1H), 7.56-7.42 (m, 2H), 7.29-7.14 (m, 3H), 7.10 (d, J=6.8 Hz, 1H), 6.95-6.82 (m, 3H), 6.78 (d, J=8.4 Hz, 1H), 6.47 (t, J=5.3 Hz, 1H), 5.14 (d, J=8.0 Hz, 1H), 4.57 (d, J=5.7 Hz, 2H), 4.47-4.26 (m, 4H), 4.12-3.94 (m, 5H), 3.78-3.62 (m, 4H), 3.51 (s, 8H), 2.99-2.83 (m, 1H), 2.60 (t, J=6.8 Hz, 3H), 2.40-2.22 (m, 1H), 2.12-1.91 (m, 1H).

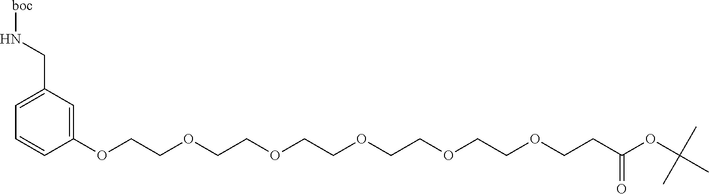

Compound 3. A solution of compound 1 (4.5 mmol, 1 eq), compound 2 (1.01 g, 4.5 mmol, 1 eq) and triphenilphosphine (1.19 g, 4.5 mmol, 1 eq) in dry THF (50 mL) under argon was cooled to 0° C., then DIAD (0.92 g, 4.5 mmol, 1 eq) was added. The mixture was stirred at rt for 16 h. THF was removed under reduced pressure, the residue was purified by column chromatography eluting with hexane-EtOAc—NH₄OH 66:33:5. Light yellow oil 77%. LCMS (C18 column 20×2 mm, 2.5 μm, pore size 100 Å, water-acetonitrile+0.1% TFA, gradient 5 to 87% for 3 min, retention time 1.64 min). MS (ESI) m/z 572.7 [MH]+.

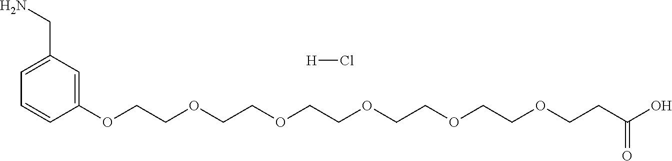

Compound 4. 100% crude. LCMS (C18 column 20×2 mm, 2.5 μm, pore size 100 Å, water-acetonitrile+0.1% TFA, gradient 5 to 87% for 3 min, retention time 1.12 min). MS (ESI) m/z 416.5 [MH]+.

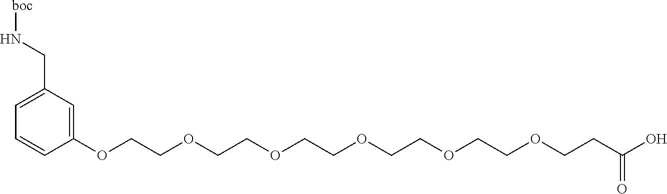

Compound 5. Light yellow oil 48%. LCMS (C18 column 20×2 mm, 2.5 μm, pore size 100 Å, water-acetonitrile+0.1% TFA, gradient 5 to 87% for 3 min, retention time 1.32 min). MS (ESI) m/z 516.5 [MH]+.

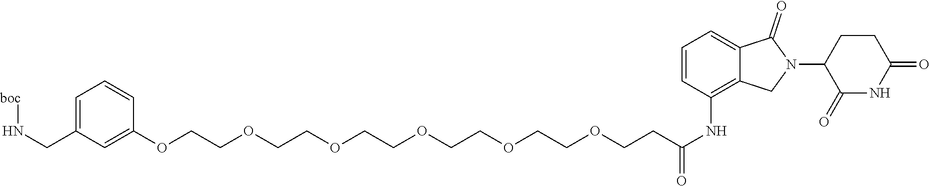

Compound 7. Light yellow oil 35%. LCMS (C18 column 20×2 mm, 2.5 μm, pore size 100 Å, water-acetonitrile+0.1% TFA, gradient 5 to 87% for 3 min, retention time 1.33 min). MS (ESI) m/z 757.8 [MH]+.

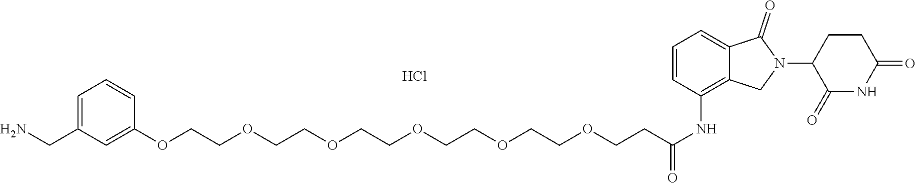 [SMILES: NCc1cccc(OCCOCCOCCOCCOCCOCCC(=O)Nc2cccc3c2CN(C2CCC(=O)NC2=O)C3=O)c1 ; Cl]

Compound 8. 95% crude. LCMS (C18 column 20×2 mm, 2.5 μm, pore size 100 Å, water-acetonitrile+0.1% TFA, gradient 5 to 87% for 3 min, retention time 1.01 min). MS (ESI) m/z 657.8 [MH]+.

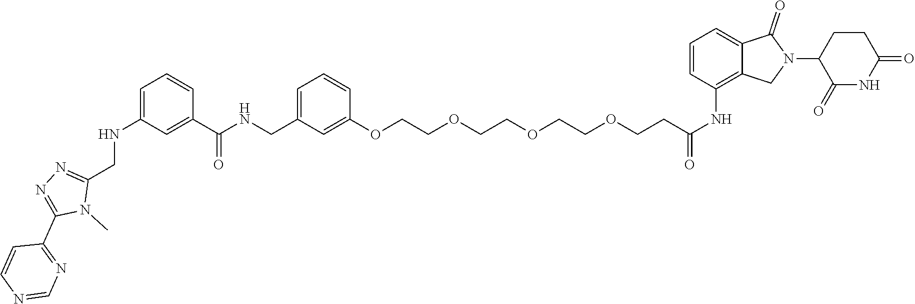 [SMILES: Cn1c(CNc2cccc(C(=O)NCc3cccc(OCCOCCOCCOCCC(=O)Nc4cccc5c4CN(C4CCC(=O)NC4=O)C5=O)c3)c2)nnc1-c1ccncn1]

Compound 9. D107. Yellow powder 42%. LCMS (C18 column 100×4.6 mm, 5.0 μm, pore size 100 Å, water-acetonitrile+0.1% TFA, gradient 5 to 87% for 10 min, retention time 4.90 min). MS (ESI) m/z 950.0 [MH]+. ¹H NMR (400 MHz, DMSO-d₆) δ 11.01 (s, 1H), 9.82 (s, 1H), 9.33 (s, 1H), 8.97 (d, J=5.3 Hz, 1H), 8.81 (t, J=6.1 Hz, 1H), 8.17 (dd, J=5.2, 1.3 Hz, 1H), 7.81 (d, J=7.1 Hz, 1H), 7.49 (q, J=7.6 Hz, 2H), 7.28-7.14 (m, 3H), 7.10 (d, J=7.5 Hz, 1H), 6.95-6.83 (m, 3H), 6.80 (d, J=8.0 Hz, 1H), 6.47 (t, J=5.5 Hz, 1H), 5.14 (dd, J=13.2, 5.0 Hz, 1H), 4.57 (d, J=5.6 Hz, 2H), 4.41 (d, J=5.7 Hz, 2H), 4.35 (d, J=9.2 Hz, 2H), 4.11-4.00 (m, 5H), 3.76-3.66 (m, 4H), 3.60-3.40 (m, 16H), 2.98-2.84 (m, 1H), 2.65-2.54 (m, 3H), 2.40-2.23 (m, 1H), 2.09-1.95 (m, 1H).

### Example 13: Preparation of Intermediates

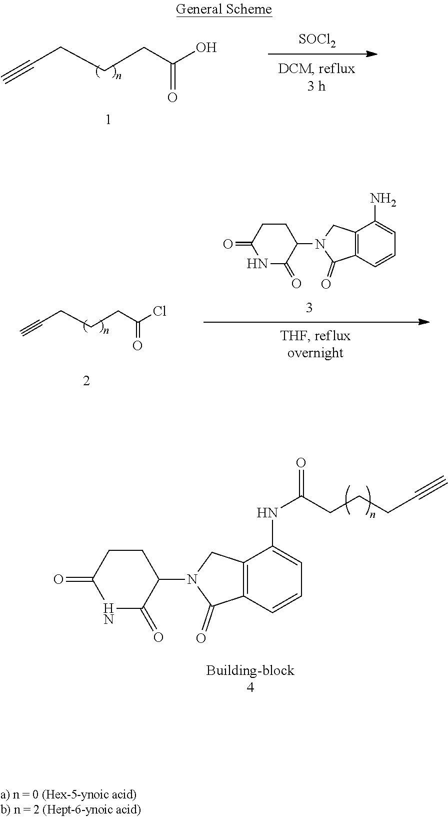

### Procedures and Analytical Data

Compound 2a. Thionyl chloride (0.012 mol) was added dropwise to a solution of acid 1a (0.011 mol) in DCM (10 mL) at rt. The mixture was shaken for 3 h at reflux. Control by TLC. The solvent was removed in vacuo and the product 2a used to next study without purification.

Compound 4a. Chloroanhydride 2a (0.011 mol) and compound 3 (0.004 mol) in THF (20 mL) were stirred at reflux for overnight. Pure 4a was isolated as white powder by filtration of reaction mixture. The yield of 4a was 47%. LCMS (C18 column 20×2 mm, 2.5 μm, pore size 100 Å, water-acetonitrile+0.1% TFA, gradient 5 to 87% for 3 min, retention time 0.94 min). MS (ESI) m/z 354.6 [MH]+ (calculated for C₁₉H₁₉N₃O₄ 353.38). ¹H NMR (400 MHz, DMSO-d₆) δ 11.01 (s, 1H), 9.82 (s, 1H), 7.90-7.74 (m, 1H), 7.57-7.42 (m, 2H), 5.23-5.06 (m, 1H), 4.37 (q, J=17.0 Hz, 2H), 2.99-2.78 (m, 2H), 2.67-2.55 (m, 1H), 2.48-2.44 (m, 1H), 2.45-2.15 (m, 4H), 2.12-1.92 (m, 1H), 1.86-1.67 (m, 2H).

The compound 4b was obtained according to this procedure from acid 1b as SM1. The yield of 4b was 97%. LCMS (C18 column 20×2 mm, 2.5 μm, pore size 100 Å, water-acetonitrile+0.1% TFA, gradient 5 to 87% for 3 min, retention time 0.98 min). MS (ESI) m/z 368.5 [MH]+(calculated for C₂₀H₂₁N₃O₄ 367.41). ¹H NMR (400 MHz, DMSO-d₆) δ 11.01 (s, 1H), 9.78 (s, 1H), 7.87-7.75 (m, 1H), 7.58-7.42 (m, 2H), 5.23-5.06 (m, 1H), 4.35 (q, J=17.4 Hz, 2H), 2.99-2.85 (m, 1H), 2.81-2.72 (m, 1H), 2.66-2.56 (m, 1H), 3.34-2.27 (m, 3H), 2.26-2.15 (m, 2H), 2.09-1.97 (m, 1H), 1.78-1.62 (m, 2H), 1.57-1.44 (m, 2H).

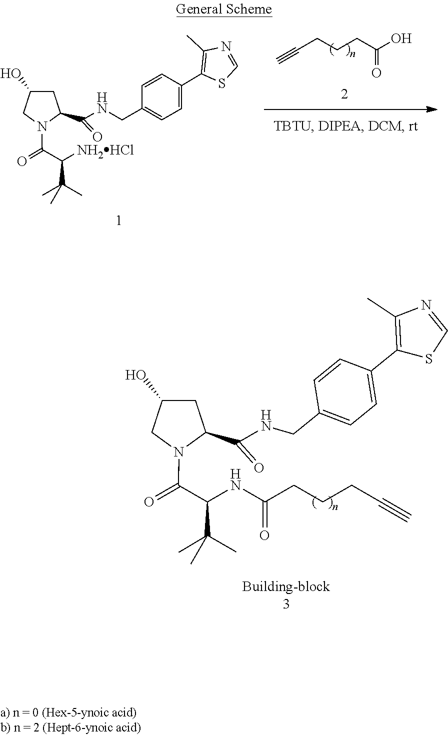

### Procedures and Analytical Data

Compound 3a. To a solution of compound 1 (0.64 mmol), acid 2a (0.70 mmol) and DIPEA (1.92 mmol) in DCM (5 mL) was added TBTU (0.96 mmol) and reaction mixture was stirred for 16 h at RT. The reaction mixture was dissolved in water. The aqueous layer was extracted with DCM and the combined organic phases were dried Na₂SO₄. The solvent was removed in vacuo and the product was purified by silica gel column chromatography with chloroform/MeOH-2%. The yield of 3a was 89%. LCMS (C18 column 20×2 mm, 2.5 μm, pore size 100 Å, water-acetonitrile+0.1% TFA, gradient 5 to 87% for 3 min, retention time 1.14 min). MS (ESI) m/z 525.6 [MH]+(calculated for C₂₈H₃₆N₄O₄S 524.69).

The compound 3b was obtained according to this procedure from acid 2b as SM2. The yield of 3b was 98%. LCMS (C18 column 20×2 mm, 2.5 μm, pore size 100 Å, water-acetonitrile+0.1% TFA, gradient 5 to 87% for 3 min, retention time 1.20 min). MS (ESI) m/z 539.7 [MH]+(calculated for C₂₉H₃₈N₄O₄S 538.71).

### Example 14: Preparation of Compounds D86 and D63

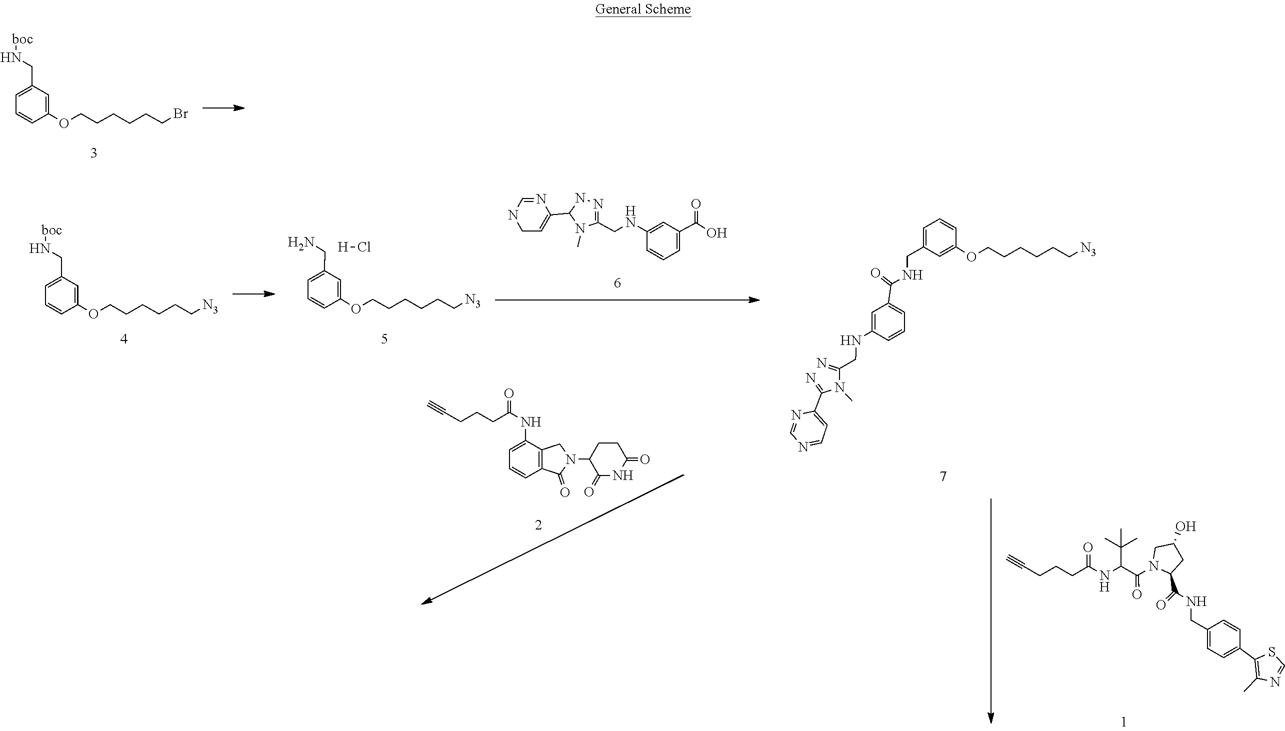

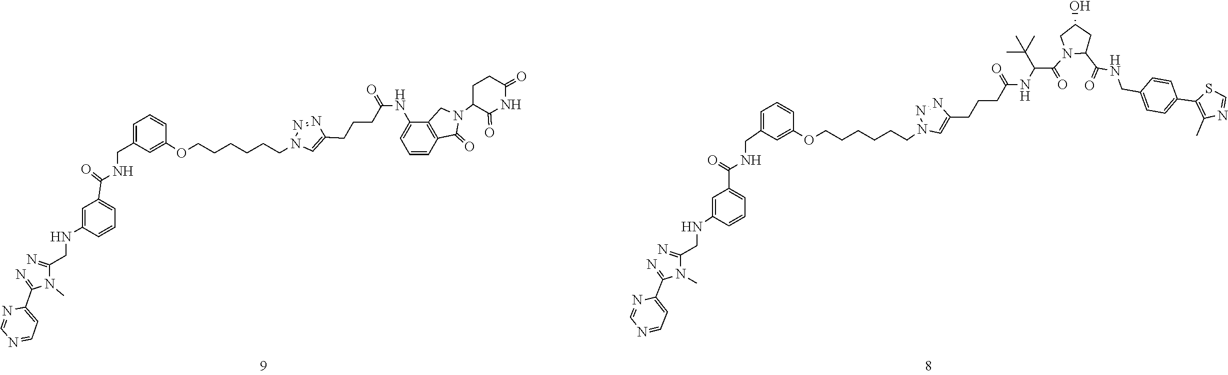 [SMILES: Cn1c(CNc2cccc(C(=O)NCc3cccc(OCCCCCCn4cc(CCCC(=O)Nc5cccc6c5CN(C5CCC(=O)NC5=O)C6=O)nn4)c3)c2)nnc1-c1ccncn1 ; Cc1ncsc1-c1ccc(CNC(=O)C2C[C@@H](O)CN2C(=O)C(NC(=O)CCCc2cn(CCCCCCOc3cccc(CNC(=O)c4cccc(NCc5nnc(-c6ccncn6)n5C)c4)c3)nn2)C(C)(C)C)cc1]

### Compounds, Procedures, and Analytical Data

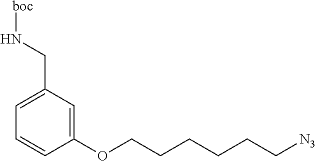

To a solution of bromide 3 (5.3 g, 13.7 mmol) in dried DMF (10 ml) NaN₃ (1.8 g, 27.7 mmol) was added and the reaction mixture was stirred at ambient temperature overnight. Then the resulted slurry was poured onto water (100 mL) and the product was extracted with ethyl acetate (3 times by 20 mL). Combined organic layers were washed with water (3 times by 20 mL) and then once with brine (10 mL). After drying over MgSO₄ the solvent was stripped off and product was used further without purification. Yield 2.4 g (50%). Colorless oil 50%. LCMS (C18 column 20×2 mm, 2.5 μm, pore size 100 Å, water-acetonitrile+0.1% TFA, gradient 5 to 87% for 3 min, retention time 2.32 min). MS (ESI) m/z 349.6 [MH]+. ¹H NMR (400 MHz, CDCl₃) δ 7.24 (t, J=7.8 Hz, 1H), 6.90-6.75 (m, 3H), 4.84 (s, 1H), 4.29 (d, J=4.7 Hz, 2H), 3.96 (t, J=6.4 Hz, 2H), 3.30 (t, J=6.8 Hz, 2H), 1.80 (dd, J=14.1, 6.6 Hz, 2H), 1.66 (dd, J=14.2, 7.0 Hz, 2H), 1.56-1.44 (m, 13H).

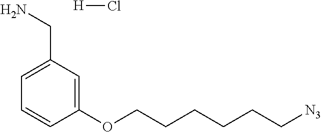

To an ice cold solution of azide 4 (2.2 g, 6.32 mmol) in methylene chloride (20 ml) a dioxanic 3M HCl solution (10 mL) was added. The mixture was stirred at ambient temperature overnight and then evaporated dryness. The residue was taken up with dry ether (20 mL) and the solid was filtered, washed with ether (10 mL) and dried on air. Yield 1.77 g (98%). Colorless solid 98%. ¹H NMR (400 MHz, DMSO-d₆) δ 8.47 (s, 3H), 7.30 (t, J=7.9 Hz, 1H), 7.12 (s, 1H), 7.02 (d, J=7.4 Hz, 1H), 6.92 (d, J=8.2 Hz, 1H), 3.97 (t, J=6.2 Hz, 4H), 1.80-1.64 (m, 2H), 1.57 (dd, J=14.0, 6.9 Hz, 2H), 1.41 (t, J=10.9 Hz, 4H).

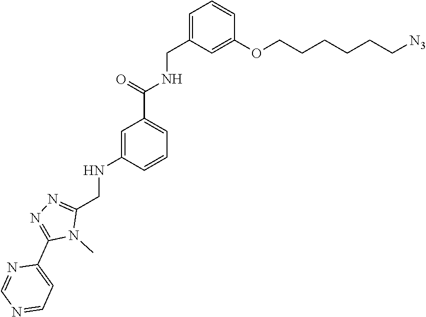 [SMILES: Cn1c(CNc2cccc(C(=O)NCc3cccc(OCCCCCCN=[N+]=[N-])c3)c2)nnc1-c1ccncn1]

To an ice cold suspension of amine hydrochloride 5 (0.46 g, 1.6 mmol) in methylene chloride (5 mL), acid 6 (0.5 g, 1.6 mmol) was added, followed by hydroxyl benzotriazole hydrate (0.27 g, 1.7 mmol) and EDCI (0.34 g, 1.8 mmol) and finally DIPEA (0.85 mL, 0.63 g, 4.9 mmol). The reaction mixture was stirred at ambient temperature overnight. Then water (5 mL) was added and organic layer was separated. Aqueous layer was extracted with methylene chloride (5 mL). Combined organic layers were dried over MgSO₄ and evaporated. The residue was purified by chromatography on silica, using chloroform-methanol (20 to 1) mixture as an eluent. Yield 0.79 g (90%). Colorless oil 50%. LCMS (C18 column 20×2 mm, 2.5 μm, pore size 100 Å, water-acetonitrile+0.1% TFA, gradient 5 to 87% for 3 min, retention time 1.59 min). MS (ESI) m/z 541.7 [MH]+. ¹H NMR (400 MHz, CDCl₃) δ 9.27 (s, 1H), 8.86 (d, J=5.2 Hz, 1H), 8.25 (d, J=5.2 Hz, 1H), 7.36-7.17 (m, 4H), 7.10 (d, J=7.6 Hz, 1H), 6.97-6.86 (m, 3H), 6.82 (d, J=8.2 Hz, 1H), 6.61 (s, 1H), 4.68-4.48 (m, 4H), 4.16 (s, 3H), 3.95 (t, J=6.3 Hz, 2H), 3.28 (t, J=6.9 Hz, 2H), 1.87-1.70 (m, 2H), 1.68-1.55 (m, 2H), 1.55-1.35 (m, 4H).

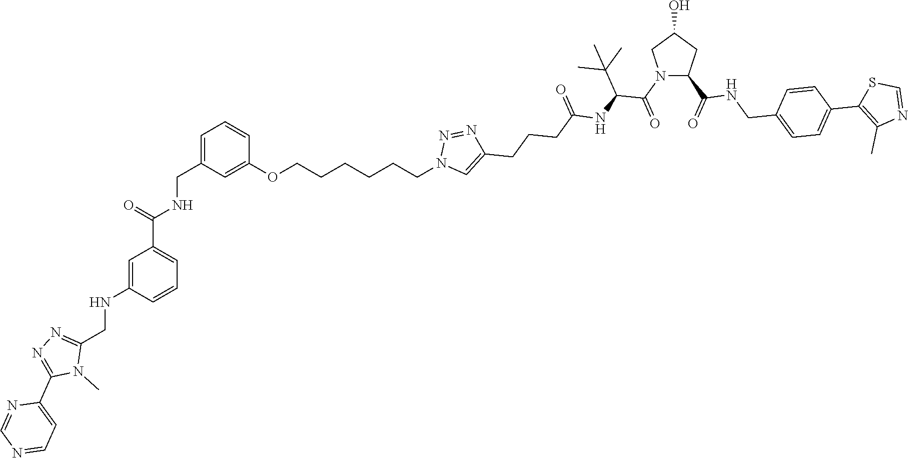 [SMILES: Cc1ncsc1-c1ccc(CNC(=O)[C@@H]2C[C@@H](O)CN2C(=O)[C@@H](NC(=O)CCCc2cn(CCCCCCOc3cccc(CNC(=O)c4cccc(NCc5nnc(-c6ccncn6)n5C)c4)c3)nn2)C(C)(C)C)cc1]

D86. To a solution of azide 7 (0.05 g, 0.09 mmol) and alkyne 1 (0.049 g, 0.09 mmol) in ethanol (1 mL), sodium ascorbate (2 mg, 0.01 mmol) in water (0.2 mL) and copper (II) acetate (2.2 mg, 0.01 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After evaporation the product was purified by chromatography on silica, using chloroform-methanol (20 to 1, then 10 to 1) mixture as an eluent. Yield 0.09 g (91%). Colorless oil 91%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 1065.8 [MH]+. ¹H NMR (400 MHz, DMSO-d₆) δ 9.33 (s, 1H), 8.98 (s, 2H), 8.81 (t, J=5.2 Hz, 1H), 8.55 (t, J=5.2 Hz, 1H), 8.17 (d, J=5.2 Hz, 1H), 7.89 (d, J=9.0 Hz, 1H), 7.83 (s, 1H), 7.40 (q, J=8.2 Hz, 4H), 7.24-7.16 (m, 3H), 7.10 (d, J=7.5 Hz, 1H), 6.90 (d, J=7.9 Hz, 1H), 6.86-6.84 (m, 2H), 6.77 (d, J=7.4 Hz, 1H), 6.47 (t, J=5.2 Hz, 1H), 5.11 (s, 1H), 4.58-4.54 (m, 3H), 4.45-4.38 (m, 4H), 4.35 (s, 1H), 4.29-4.15 (m, 3H), 4.07 (s, 3H), 3.90 (t, J=5.2 Hz, 2H), 3.66 (s, 2H), 2.57 (t, J=5.2 Hz, 2H), 2.44 (s, 3H), 2.34-2.27 (m, 1H), 2.23-2.15 (m, 1H), 2.07-1.99 (m, 1H), 1.94-1.87 (m, 1H), 1.84-1.71 (m, 4H), 1.72-1.61 (m, 2H), 1.46-1.36 (m, 2H), 1.31-1.21 (m, 2H), 0.99-0.88 (m, 9H).

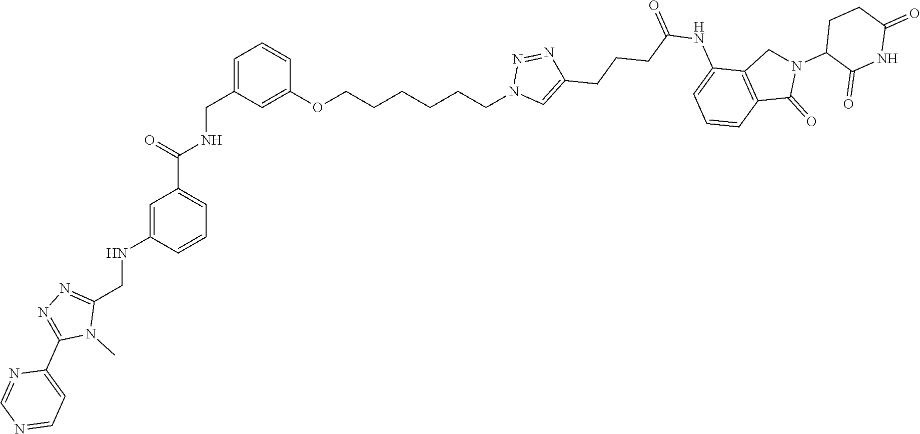 [SMILES: Cn1c(CNc2cccc(C(=O)NCc3cccc(OCCCCCCn4cc(CCCC(=O)Nc5cccc6c5CN(C5CCC(=O)NC5=O)C6=O)nn4)c3)c2)nnc1-c1ccncn1]

D63. To a solution of azide 7 (0.05 g, 0.09 mmol) and alkyne 1 (0.049 g, 0.09 mmol) in ethanol (1 mL), sodium ascorbate (2 mg, 0.01 mmol) in water (0.2 mL) and copper (II) acetate (2.2 mg, 0.01 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After evaporation the product was purified by chromatography on silica, using chloroform-methanol (20 to 1, then 10 to 1) mixture as an eluent. Yield 0.09 g (91%). Colorless oil 91%. LCMS (C18 column 20×2 mm, 2.5 μm, pore size 100 Å, water-acetonitrile+0.1% TFA, gradient 5 to 87% for 10 min, retention time 5.39 min). MS (ESI) m/z 895.4 [MH]+. ¹H NMR (400 MHz, DMSO-d₆) δ 11.00 (s, 1H), 9.78 (s, 1H), 9.33 (s, 1H), 8.97 (d, J=5.3 Hz, 1H), 8.81 (t, J=5.3 Hz, 1H), 8.17 (d, J=5.3 Hz, 1H), 7.87 (s, 1H), 7.81 (d, J=7.0 Hz, 1H), 7.53-7.42 (m, 2H), 7.26-7.12 (m, 3H), 7.10 (d, J=8.1 Hz, 1H), 6.90 (d, J=8.1 Hz, 1H), 6.86-6.84 (m, 2H), 6.77 (d, J=9.3 Hz, 1H), 6.47 (t, J=5.3 Hz, 1H), 5.14 (dd, J=13.1, 5.1 Hz, 1H), 4.57 (d, J=5.4 Hz, 2H), 4.40 (d, J=5.8 Hz, 2H), 4.36 (d, J=13.1 Hz, 2H), 4.29 (t, J=5.8 Hz, 2H), 4.07 (s, 3H), 3.90 (t, J=6.4 Hz, 2H), 2.96-2.85 (m, 1H), 2.64 (t, J=7.5 Hz, 2H), 2.59 (d, J=13.1 Hz, 1H), 2.41 (t, J=7.5 Hz, 2H), 2.35 (d, J=13.1 Hz, 1H), 2.05-1.98 (m, 1H), 1.96-1.89 (m, 2H), 1.83-1.76 (m, 2H), 1.70-1.62 (m, 2H), 1.45-1.36 (m, 2H), 1.31-1.22 (m, 2H).

### Example 15: Preparation of Compounds D81 and D59

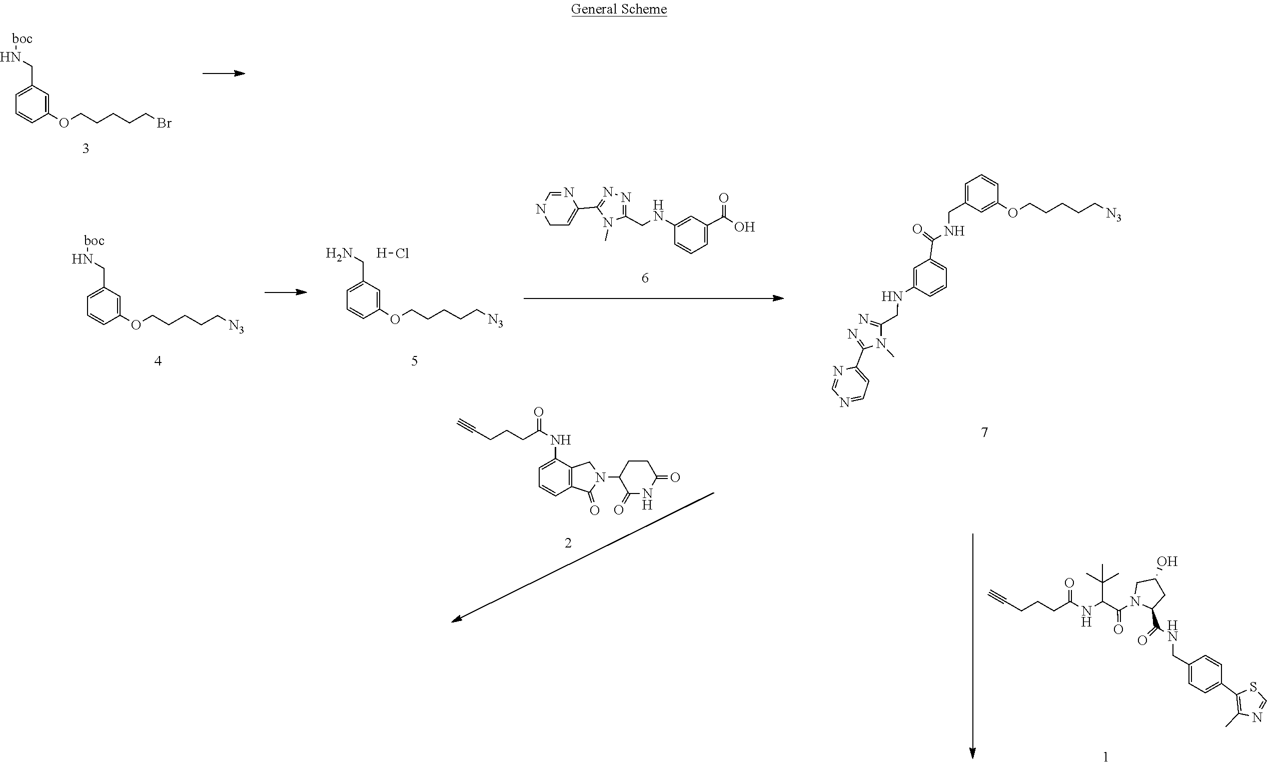

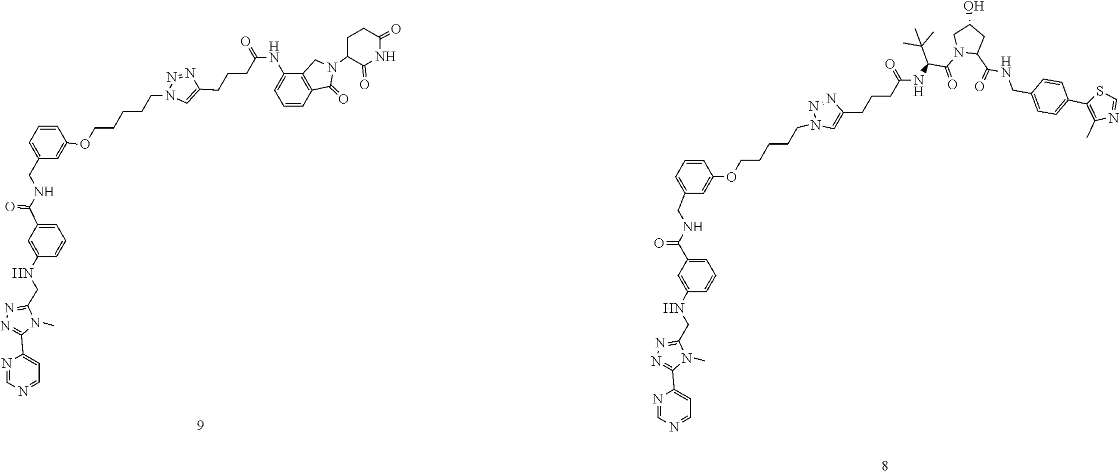 [SMILES: Cn1c(CNc2cccc(C(=O)NCc3cccc(OCCCCCn4cc(CCCC(=O)Nc5cccc6c5CN(C5CCC(=O)NC5=O)C6=O)nn4)c3)c2)nnc1-c1ccncn1 ; Cc1ncsc1-c1ccc(CNC(=O)C2C[C@@H](O)CN2C(=O)[C@@H](NC(=O)CCCc2cn(CCCCCOc3cccc(CNC(=O)c4cccc(NCc5nnc(-c6ccncn6)n5C)c4)c3)nn2)C(C)(C)C)cc1]

### Compounds, Procedures, and Analytical Data

To a solution of azide 7 (0.05 g, 0.09 mmol) and alkyne 1 (0.049 g, 0.09 mmol) in ethanol (1 mL), sodium ascorbate (2 mg, 0.01 mmol) in water (0.2 mL) and copper (II) acetate (2.2 mg, 0.01 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After evaporation the product was purified by chromatography on silica, using chloroform-methanol (20 to 1, then 10 to 1) mixture as an eluent. Yield 0.09 g (91%).

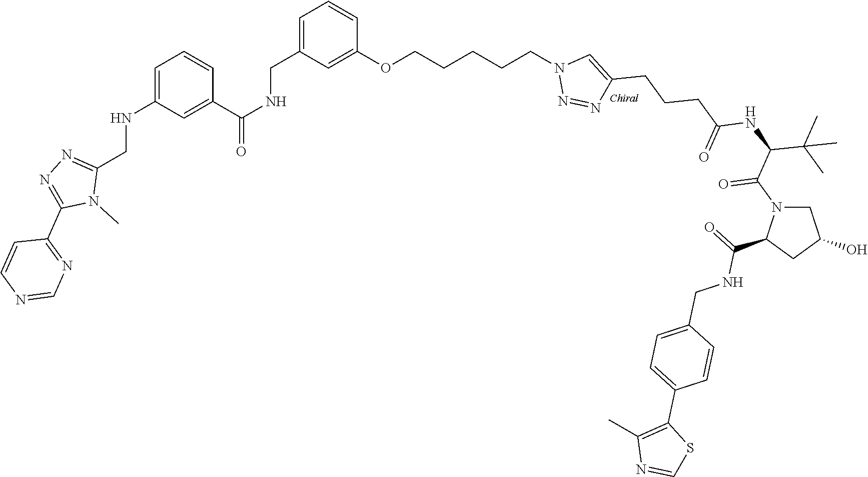 [SMILES: Cc1ncsc1-c1ccc(CNC(=O)[C@@H]2C[C@@H](O)CN2C(=O)[C@@H](NC(=O)CCCc2cn(CCCCCOc3cccc(CNC(=O)c4cccc(NCc5nnc(-c6ccncn6)n5C)c4)c3)nn2)C(C)(C)C)cc1]

D81. Colorless oil 91%. LCMS (C18 column 20×2 mm, 2.5 μm, pore size 100 Å, water-acetonitrile+0.1% TFA, gradient 5 to 87% for 10 min, retention time 5.71 min). MS (ESI) m/z 1092.1 [MH]+. ¹H NMR (400 MHz, DMSO-d₆) δ 9.33 (s, 1H), 9.01-8.93 (m, 2H), 8.81 (t, J=7.2 Hz, 1H), 8.55 (t, J=7.2 Hz, 1H), 8.17 (d, J=5.3 Hz, 1H), 7.96-7.81 (m, 2H), 7.39 (q, J=8.3 Hz, 4H), 7.29-7.16 (m, 3H), 7.10 (d, J=7.7 Hz, 1H), 6.96-6.81 (m, 3H), 6.77 (d, J=7.8 Hz, 1H), 6.47 (t, J=5.5 Hz, 1H), 5.12 (d, J=3.5 Hz, 1H), 4.64-4.50 (m, 3H), 4.45-4.17 (m, 8H), 4.07 (s, 3H), 3.91 (t, J=6.5 Hz, 2H), 3.66 (s, 2H), 2.57 (t, J=7.6 Hz, 2H), 2.44 (s, 3H), 2.42-2.26 (m, 1H), 2.23-2.15 (m, 1H), 2.05-1.95 (m, 1H), 1.92-1.56 (m, 7H), 1.56-1.33 (m, 2H), 0.94 (s, 9H).

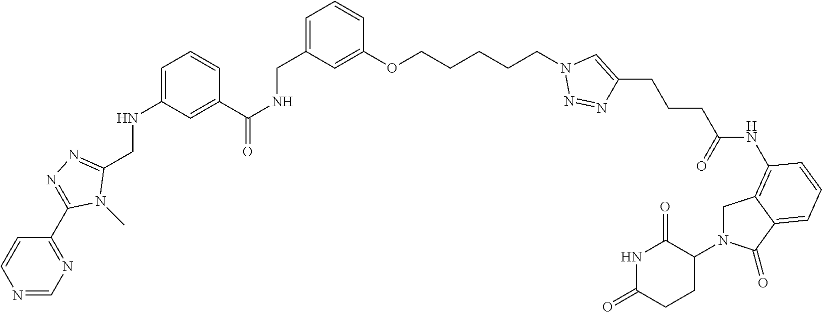 [SMILES: Cn1c(CNc2cccc(C(=O)NCc3cccc(OCCCCCn4cc(CCCC(=O)Nc5cccc6c5CN(C5CCC(=O)NC5=O)C6=O)nn4)c3)c2)nnc1-c1ccncn1]

D59. Colorless oil 91%. LCMS (C18 column 20×2 mm, 2.5 μm, pore size 100 Å, water-acetonitrile+0.1% TFA, gradient 5 to 87% for 10 min, retention time 5.20 min). MS (ESI) m/z 881.1 [MH]+. ¹H NMR (400 MHz, DMSO-d₆) δ 11.01 (s, 1H), 9.78 (s, 1H), 9.33 (s, 1H), 8.96 (d, J=5.3 Hz, 1H), 8.81 (t, J=7.0 Hz, 1H), 8.17 (d, J=5.2 Hz, 1H), 7.87 (s, 1H), 7.81 (d, J=7.2 Hz, 1H), 7.56-7.40 (m, 2H), 7.28-7.14 (m, 3H), 7.10 (d, J=7.1 Hz, 1H), 6.94-6.81 (m, 3H), 6.77 (d, J=7.6 Hz, 1H), 6.47 (t, J=5.5 Hz, 1H), 5.13 (d, J=8.7 Hz, 1H), 4.57 (d, J=5.6 Hz, 2H), 4.47-4.34 (m, 4H), 4.30 (t, J=7.0 Hz, 2H), 4.07 (s, 3H), 3.91 (t, J=6.3 Hz, 2H), 3.01-2.85 (m, 1H), 2.72-2.55 (m, 3H), 2.46-2.26 (m, 3H), 2.09-1.97 (m, 1H), 1.97-1.89 (m, 2H), 1.89-1.77 (m, 2H), 1.77-1.64 (m, 2H), 1.44-1.27 (m, 2H).

### Example 16: Preparation of Compounds D84 and D62

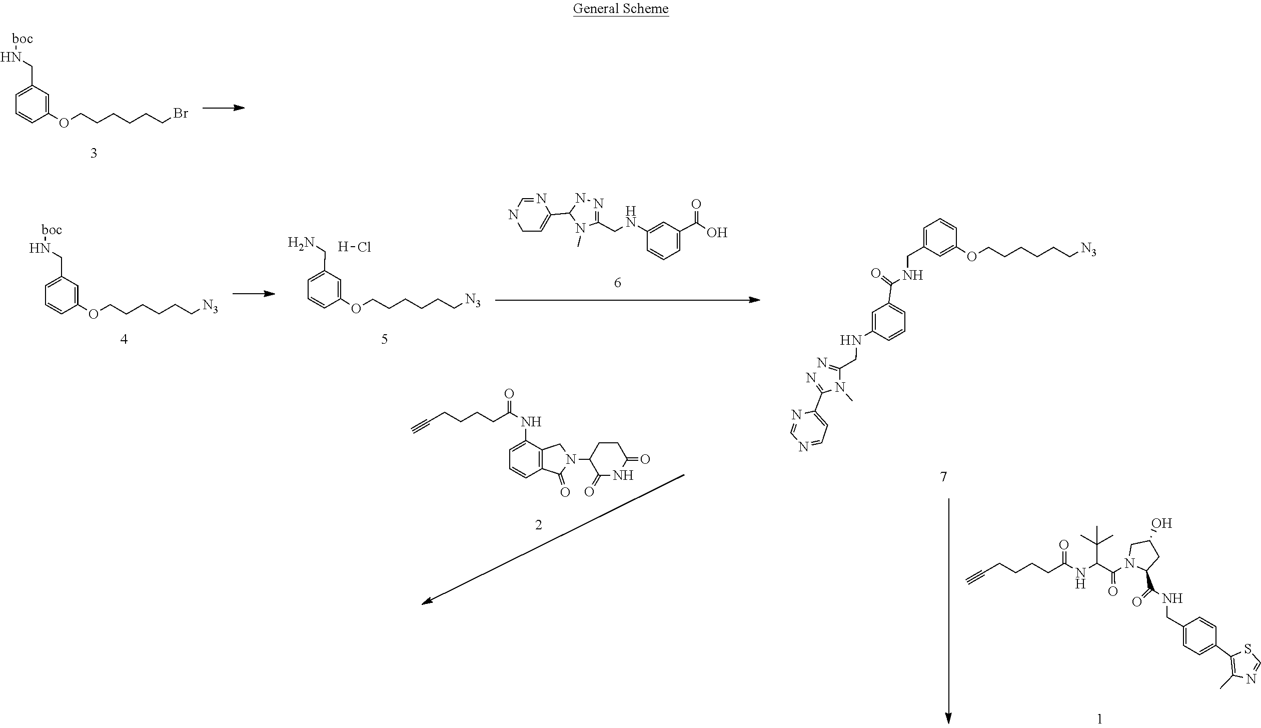

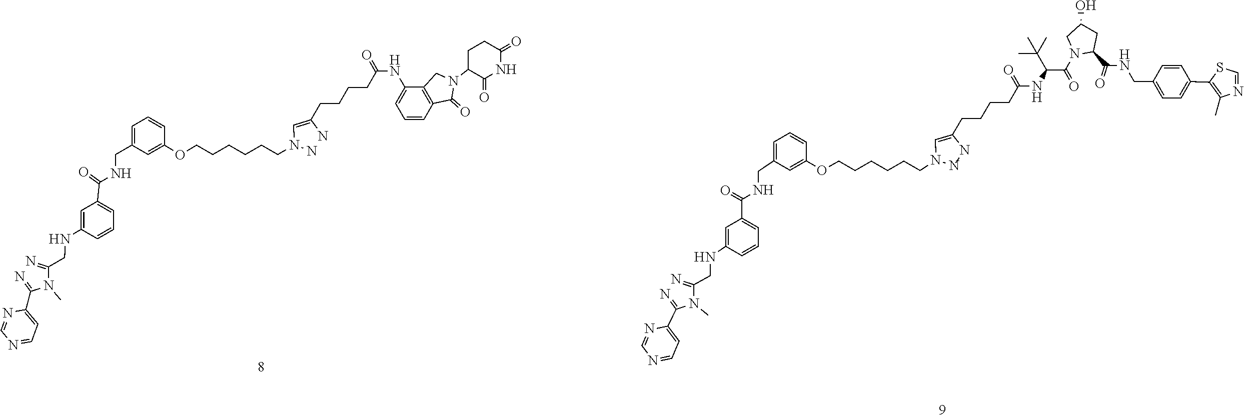

### Compounds, Procedures, and Analytical Data

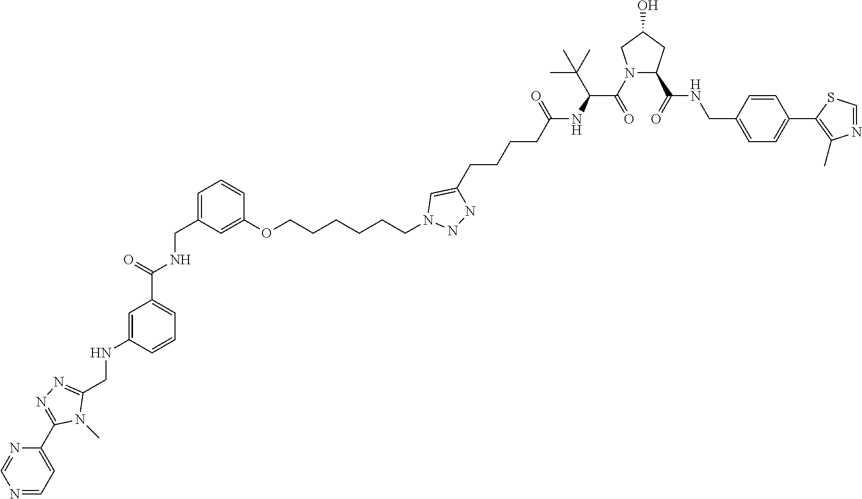 [SMILES: Cc1ncsc1-c1ccc(CNC(=O)[C@@H]2C[C@@H](O)CN2C(=O)[C@@H](NC(=O)CCCCC2=CN(CCCCCCOc3cccc(CNC(=O)c4cccc(NCc5nnc(-c6ccncn6)n5C)c4)c3)NN2)C(C)(C)C)cc1]

D84. Colorless oil 91%. LCMS (C18 column 20×2 mm, 2.5 μm, pore size 100 Å, water-acetonitrile+0.1% TFA, gradient 5 to 87% for 10 min, retention time 6.76 min). MS (ESI) m/z 1079.9 [MH]+. ¹H NMR (400 MHz, DMSO-d₆) δ 9.33 (s, 1H), 8.97 (s, 1H), 8.96 (d, J=6.1 Hz, 1H), 8.81 (t, J=5.2 Hz, 1H), 8.55 (t, J=5.2 Hz, 1H), 8.17 (d, J=5.2 Hz, 1H), 7.85 (d, J=9.2 Hz, 1H), 7.80 (s, 1H), 7.40 (q, J=8.3 Hz, 4H), 7.24 (s, 1H), 7.19 (dd, J=17.2, 8.2 Hz, 2H), 7.10 (d, J=7.2 Hz, 1H), 6.90 (d, J=6.6 Hz, 1H), 6.85 (d, J=7.8 Hz, 1H), 6.84 (s, 1H), 6.77 (d, J=9.1 Hz, 1H), 6.47 (t, J=5.2 Hz, 1H), 5.12 (d, J=3.6 Hz, 1H), 4.57 (d, J=5.7 Hz, 2H), 4.53 (d, J=9.3 Hz, 1H), 4.47-4.37 (m, 4H), 4.35 (s, 1H), 4.27 (t, J=6.9 Hz, 2H), 4.21 (d, J=10.7 Hz, 1H), 4.07 (s, 3H), 3.90 (t, J=6.5 Hz, 2H), 3.65 (s, 2H), 2.58 (t, J=5.7 Hz, 2H), 2.44 (s, 3H), 2.33-2.24 (m, 1H), 2.19-2.10 (m, 1H), 2.06-1.98 (m, 1H), 1.94-1.85 (m, 1H), 1.82-1.75 (m, 2H), 1.70-1.62 (m, 2H), 1.57-1.47 (m, 4H), 1.44-1.35 (m, 2H), 1.30-1.20 (m, 2H), 0.96-0.88 (m, 9H).

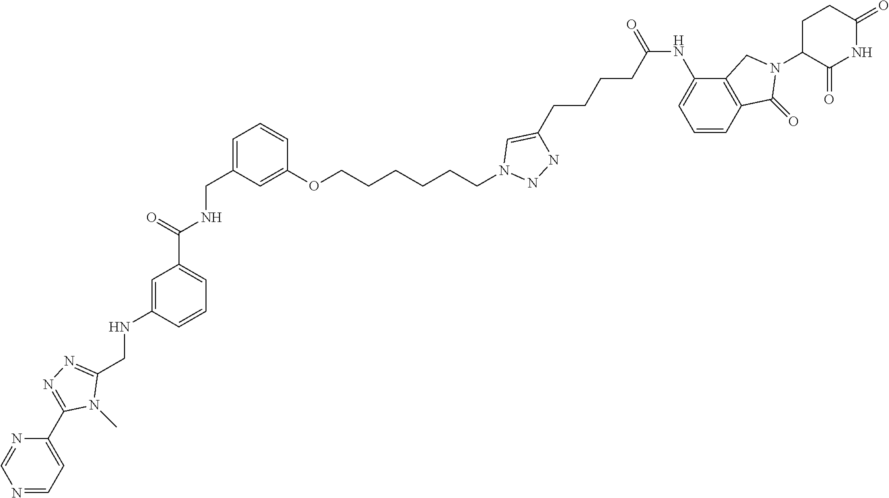 [SMILES: Cn1c(CNc2cccc(C(=O)NCc3cccc(OCCCCCCN4C=C(CCCCC(=O)Nc5cccc6c5CN(C5CCC(=O)NC5=O)C6=O)NN4)c3)c2)nnc1-c1ccncn1]

D62. Colorless oil 91%. LCMS (C18 column 20×2 mm, 2.5 μm, pore size 100 Å, water-acetonitrile+0.1% TFA, gradient 5 to 87% for 3 min, retention time 1.30 min). MS (ESI) m/z 908.7 [MH]+. ¹H NMR (400 MHz, DMSO-d₆) δ 11.00 (s, 1H), 9.77 (s, 1H), 9.33 (s, 1H), 8.97 (d, J=5.2 Hz, 1H), 8.81 (t, J=5.2 Hz, 1H), 8.17 (d, J=5.2 Hz, 1H), 7.83 (s, 1H), 7.80 (d, J=7.2 Hz, 1H), 7.51-7.46 (m, 2H), 7.26-7.13 (m, 3H), 7.10 (d, J=7.7 Hz, 1H), 6.90 (d, J=7.3 Hz, 1H), 6.85 (d, J=7.7 Hz, 1H), 6.84 (s, 1H), 6.77 (d, J=8.3 Hz, 1H), 6.47 (t, J=5.2 Hz, 1H), 5.14 (dd, J=13.4, 5.2 Hz, 1H), 4.57 (d, J=5.7 Hz, 2H), 4.41 (d, J=5.9 Hz, 2H), 4.36 (d, J=7.2 Hz, 2H), 4.27 (t, J=7.1 Hz, 2H), 4.07 (s, 3H), 3.90 (t, J=6.4 Hz, 2H), 2.96-2.86 (m, 1H), 2.67-2.57 (m, 3H), 2.42-2.30 (m, 3H), 2.05-1.97 (m, 1H), 1.83-1.74 (m, 2H), 1.70-1.60 (m, 6H), 1.44-1.31 (m, 2H), 1.26-1.21 (m, 2H).

### Example 17: Preparation of Compounds D80 and D58

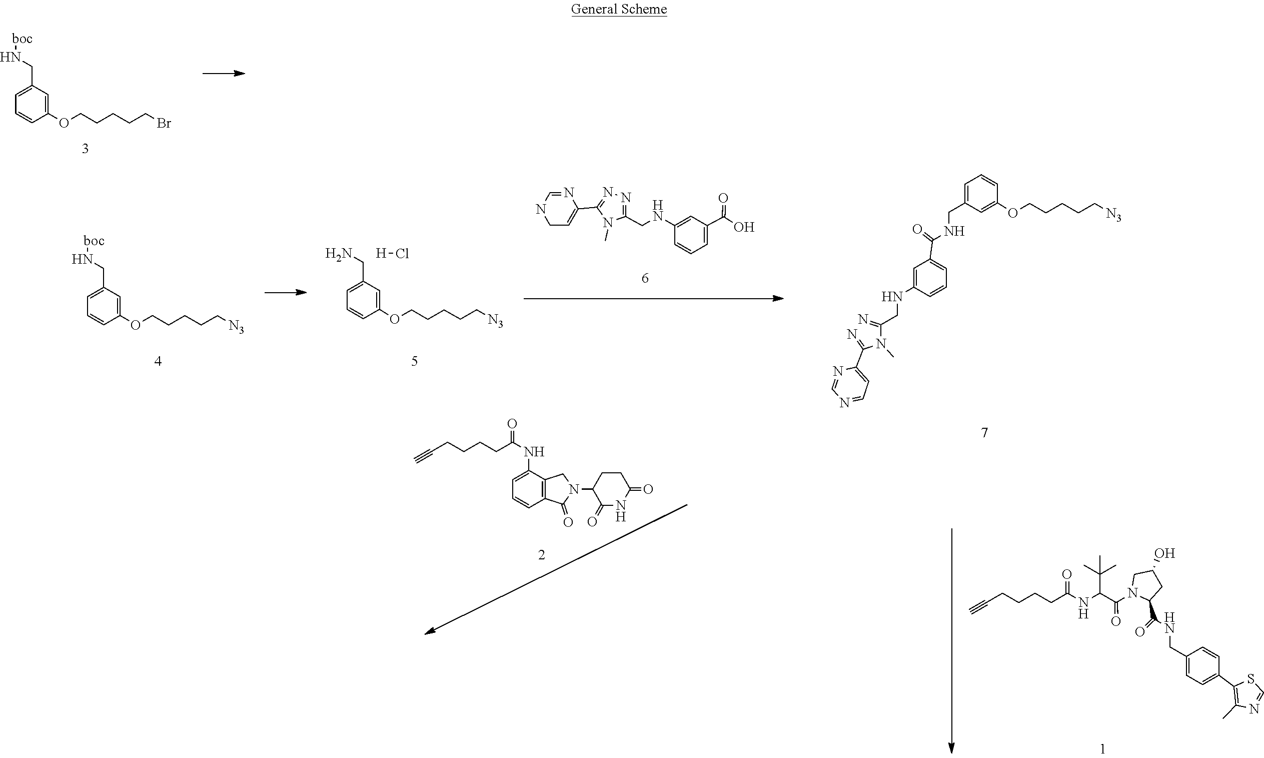

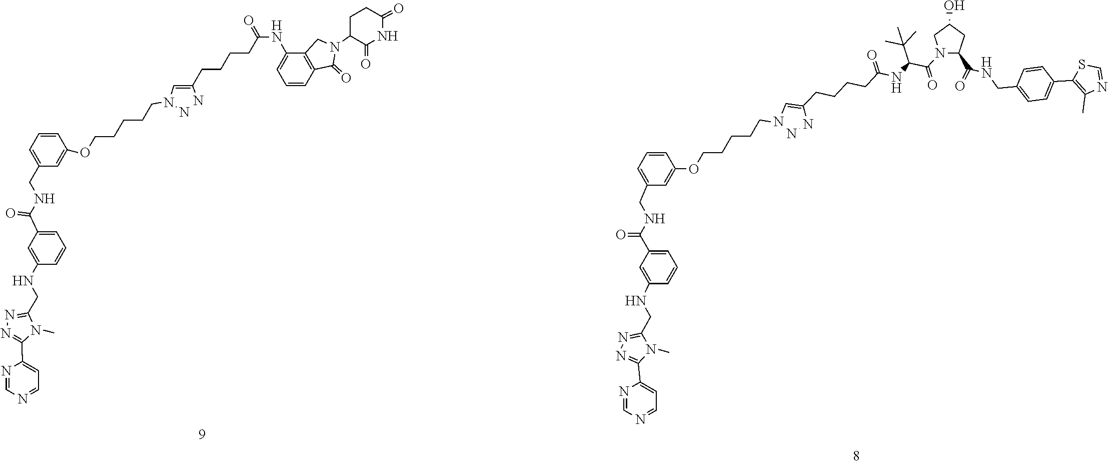

### Compounds, Procedures, and Analytical Data

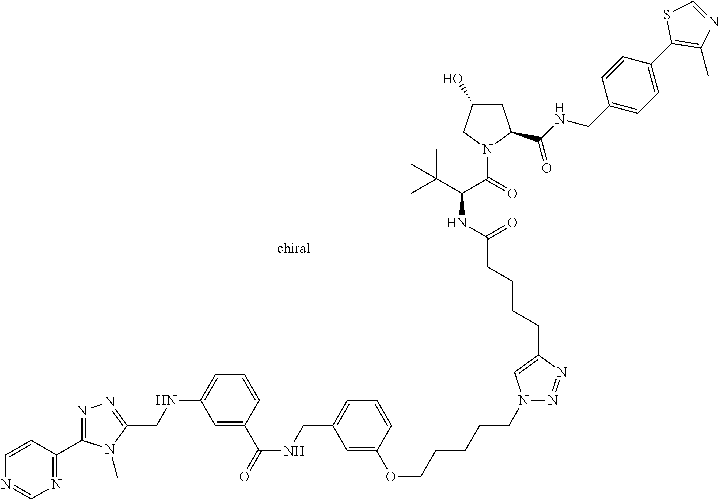 [SMILES: Cc1ncsc1-c1ccc(CNC(=O)[C@@H]2C[C@@H](O)CN2C(=O)[C@@H](NC(=O)CCCCc2cn(CCCCCOc3cccc(CNC(=O)c4cccc(NCc5nnc(-c6ccncn6)n5C)c4)c3)nn2)C(C)(C)C)cc1]

D80. Colorless oil 91%. LCMS (C18 column 20×2 mm, 2.5 μm, pore size 100 Å, water-acetonitrile+0.1% TFA, gradient 5 to 87% for 10 min, retention time 5.77 min). MS (ESI) m/z 1067.1 [MH]+. ¹H NMR (400 MHz, DMSO-d₆) δ 9.33 (s, 1H), 9.00-8.93 (m, 2H), 8.81 (t, J=6.1 Hz, 1H), 8.54 (t, J=5.7 Hz, 1H), 8.17 (d, J=5.2 Hz, 1H), 7.85 (d, J=9.3 Hz, 1H), 7.80 (s, 1H), 7.39 (q, J=8.3 Hz, 4H), 7.28-7.14 (m, 3H), 7.10 (d, J=7.8 Hz, 1H), 6.91 (d, J=8.1 Hz, 1H), 6.88-6.81 (m, 2H), 6.77 (d, J=8.1 Hz, 1H), 6.47 (t, J=5.5 Hz, 1H), 5.12 (d, J=3.5 Hz, 1H), 4.64-4.51 (m, 3H), 4.48-4.38 (m, 4H), 4.35 (s, 1H), 4.29 (t, J=7.0 Hz, 2H), 4.21 (dd, J=15.8, 5.4 Hz, 1H), 4.07 (s, 3H), 3.91 (t, J=6.5 Hz, 2H), 3.65 (s, 2H), 2.58 (t, J=7.5 Hz, 2H), 2.44 (s, 3H), 2.37-2.22 (m, 1H), 2.21-2.09 (m, 1H), 2.08-1.98 (m, 1H), 1.96-1.77 (m, 3H), 1.77-1.64 (m, J=6.9 Hz, 2H), 1.62-1.45 (m, 4H), 1.41-1.29 (m, 2H), 0.92 (s, 9H).

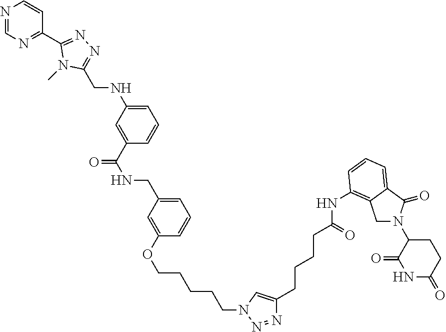 [SMILES: Cn1c(CNc2cccc(C(=O)NCc3cccc(OCCCCCn4cc(CCCCC(=O)Nc5cccc6c5CN(C5CCC(=O)NC5=O)C6=O)nn4)c3)c2)nnc1-c1ccncn1]

D58. Colorless oil 91%. LCMS (C18 column 20×2 mm, 2.5 μm, pore size 100 Å, water-acetonitrile+0.1% TFA, gradient 5 to 87% for 10 min, retention time 5.31 min). MS (ESI) m/z 895.3 [MH]+. ¹H NMR (400 MHz, DMSO-d₆) δ 11.00 (s, 1H), 9.77 (s, 1H), 9.33 (s, 1H), 8.96 (d, J=5.2 Hz, 1H), 8.81 (t, J=5.5 Hz, 1H), 8.17 (d, J=5.2 Hz, 1H), 7.84 (s, 1H), 7.80 (d, J=5.8 Hz, 1H), 7.55-7.39 (m, 2H), 7.30-7.14 (m, 3H), 7.10 (d, J=7.9 Hz, 1H), 6.90 (d, J=8.1 Hz, 1H), 6.87-6.80 (m, 2H), 6.76 (d, J=8.3 Hz, 1H), 6.48 (t, J=7.0 Hz, 1H), 5.14 (dd, J=13.4, 5.2 Hz, 1H), 4.57 (d, J=5.6 Hz, 2H), 4.44-4.32 (m, 4H), 4.29 (t, J=7.1 Hz, 2H), 4.07 (s, 3H), 3.91 (t, J=6.4 Hz, 2H), 3.01-2.84 (m, 1H), 2.73-2.55 (m, 3H), 2.44-2.26 (m, 3H), 2.11-1.94 (m, 1H), 1.91-1.77 (m, 2H), 1.76-1.56 (m, 6H), 1.47-1.20 (m, 2H).

### Example 18: Preparation of Compounds D83, D61, D158

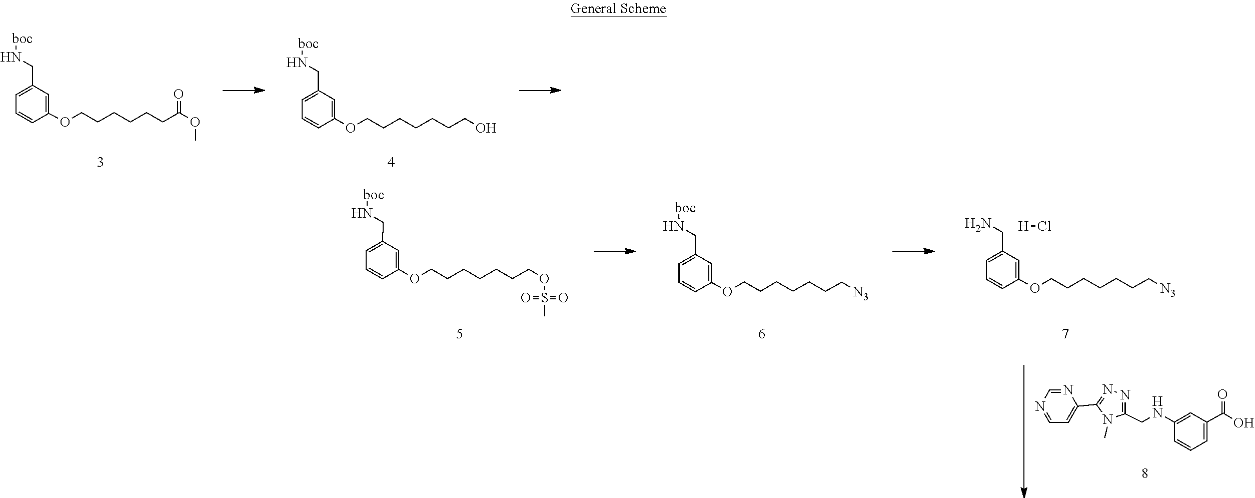

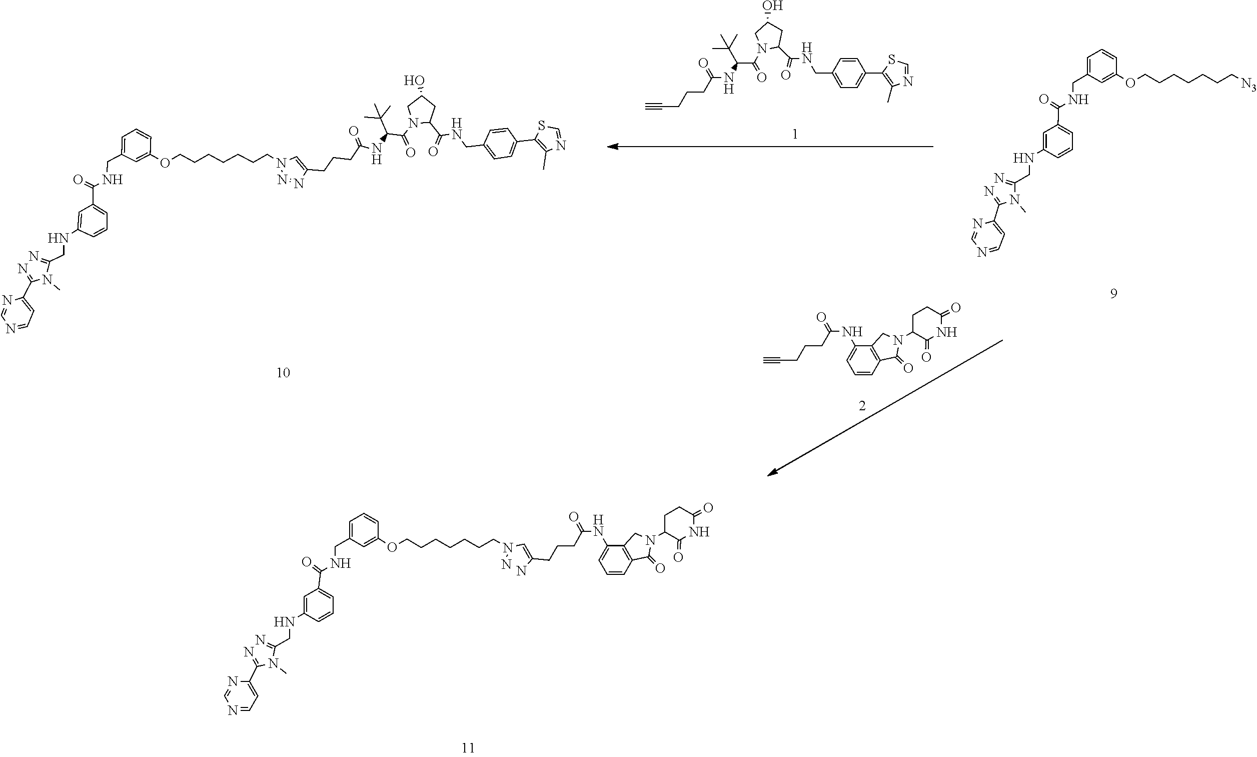 [SMILES: Cc1ncsc1-c1ccc(CNC(=O)C2C[C@@H](O)CN2C(=O)[C@@H](NC(=O)CCCc2cn(CCCCCCCOc3cccc(CNC(=O)c4cccc(NCc5nnc(-c6ccncn6)n5C)c4)c3)nn2)C(C)(C)C)cc1 ; C#CCCCC(=O)N[C@H](C(=O)N1C[C@H](O)CC1C(=O)NCc1ccc(-c2scnc2C)cc1)C(C)(C)C ; C#CCCCC(=O)Nc1cccc2c1CN(C1CCC(=O)NC1=O)C2=O ; Cn1c(CNc2cccc(C(=O)NCc3cccc(OCCCCCCCN=[N+]=[N-])c3)c2)nnc1-c1ccncn1 ; Cn1c(CNc2cccc(C(=O)NCc3cccc(OCCCCCCCn4cc(CCCC(=O)Nc5cccc6c5CN(C5CCC(=O)NC5=O)C6=O)nn4)c3)c2)nnc1-c1ccncn1]

### Compounds, Procedures, and Analytical Data

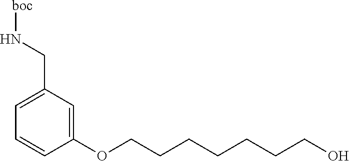

To a solution of ester 3 (3 g, 7.9 mmol) in abs THF (50 mL) LiAlH₄ (0.3 g, 7.9 mmol) at ambient temperature and the reaction mixture was stirred for 4 hours. Then the reaction was quenched with 15% aqueous NaOH (1 mL) and stirred for an hour. The solid was filtered off, washed with THF and the filtrate was evaporated dryness. The residue was purified by chromatography, using chloroform-methanol mixture (100 to 1, then 40 to 1). Yield 1.96 g (73%). Colorless oil 73%. LCMS (C18 column 20×2 mm, 2.5 μm, pore size 100 Å, water-acetonitrile+0.1% TFA, gradient 5 to 87% for 3 min, retention time 1.60 min). MS (ESI) m/z 338.5 [MH]+. ¹H NMR (400 MHz, CDCl₃) δ 7.23 (t, J=7.7 Hz, 1H), 6.82 (dd, J=19.7, 8.5 Hz, 3H), 4.86 (s, 1H), 4.29 (s, 2H), 3.96 (dd, J=8.3, 4.5 Hz, 2H), 3.79-3.57 (m, 4H), 1.78 (dd, J=14.5, 6.5 Hz, 2H), 1.74-1.64 (m, 2H), 1.59 (s, 2H), 1.47 (s, 10H), 1.43-1.38 (m, 2H).

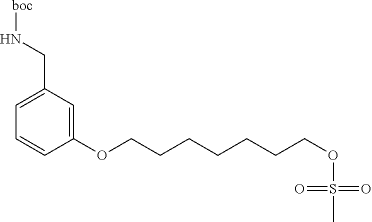

To an ice cold solution of alcohol 4 (1.96 g, 5.8 mmol) in methylene chloride (20 mL), DIPEA (2 mL, 1.5 g, 11.5 mmol) was added, followed by mesyl chloride (0.6 mL, 0.88 g, 7.73 mmol). The reaction mixture was stirred at ambient temperature for 4 hours and quenched with water (10 mL). Organic phase was separated; aqueous was extracted with methylene chloride (10 mL). Combined organic phases were washed with water (10 mL) and dried over MgSO₄. The solvent was stripped off and the product was used further without additional purification. Yield 2.4 g (100%). Colorless oil 100%. LCMS (C18 column 20×2 mm, 2.5 μm, pore size 100 Å, water-acetonitrile+0.1% TFA, gradient 5 to 87% for 3 min, retention time 1.75 min). MS (ESI) m/z 416.4 [MH]+.

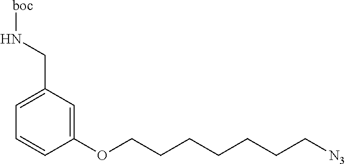

To a solution of mesylate 5 (2.4 g, 5.7 mmol) in dried DMF (10 mL) NaN₃ (0.75 g, 11.5 mmol) was added and the reaction mixture was stirred at ambient temperature overnight. Then the resulted slurry was poured onto water (100 mL) and the product was extracted with ethyl acetate (3 times by 20 mL). Combined organic layers were washed with water (3 times by 20 mL) and then once with brine (10 mL). After drying over MgSO₄ the solvent was stripped off and product was used further without purification. Yield 1.36 g (65%). Colorless oil 65%. LCMS (C18 column 20×2 mm, 2.5 μm, pore size 100 Å, water-acetonitrile+0.1% TFA, gradient 5 to 87% for 3 min, retention time 2.21 min). MS (ESI) m/z 363.6 [MH]+. ¹H NMR (400 MHz, CDCl₃) δ 7.24 (t, J=7.8 Hz, 1H), 6.89-6.77 (m, 3H), 4.84 (s, 1H), 4.30 (d, J=4.8 Hz, 2H), 3.96 (t, J=6.5 Hz, 2H), 3.28 (t, J=6.9 Hz, 2H), 1.88-1.71 (m, 2H), 1.62 (dd, J=13.9, 6.8 Hz, 2H), 1.51-1.48 (m, 11H), 1.44-1.35 (m, 4H).

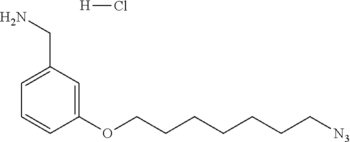

To an ice cold solution of azide 6 (1.11 g, 6.32 mmol) in methylene chloride (10 mL) a dioxanic 3M HCl solution (6 mL) was added. The mixture was stirred at ambient temperature overnight and then evaporated dryness. The residue was taken up with dry ether (20 mL) and the solid was filtered, washed with ether (10 mL) and dried on air. Yield 1.77 g (100%).

Colorless solid 100%. LCMS (C18 column 20×2 mm, 2.5 μm, pore size 100 Å, water-acetonitrile+0.1% TFA, gradient 5 to 87% for 3 min, retention time 1.29 min). MS (ESI) m/z 263.5 [MH]+.

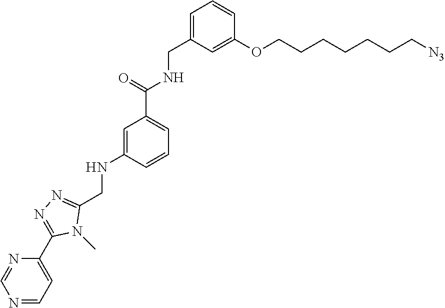 [SMILES: Cn1c(CNc2cccc(C(=O)NCc3cccc(OCCCCCCCN=[N+]=[N-])c3)c2)nnc1-c1ccncn1]

To an ice cold suspension of amine hydrochloride 7 (0.45 g, 1.5 mmol) in methylene chloride (5 mL), acid 8 (0.46 g, 1.5 mmol) was added, followed by hydroxyl benzotriazole hydrate (0.27 g, 1.7 mmol) and EDCI (0.34 g, 1.8 mmol) and finally DIPEA (0.85 mL, 0.63 g, 4.9 mmol). The reaction mixture was stirred at ambient temperature overnight. Then water (5 mL) was added and organic layer was separated. Aqueous layer was extracted with methylene chloride (5 mL). Combined organic layers were dried over MgSO₄ and evaporated. The residue was purified by chromatography on silica, using chloroform-methanol (20 to 1) mixture as an eluent. Yield 0.66 g (79%). Colorless oil 79%. LCMS (C18 column 20×2 mm, 2.5 μm, pore size 100 Å, water-acetonitrile+0.1% TFA, gradient 5 to 87% for 3 min, retention time 1.67 min). MS (ESI) m/z 555.5 [MH]+. ¹H NMR (400 MHz, CDCl₃) δ 9.27 (s, 1H), 8.86 (d, J=5.2 Hz, 1H), 8.25 (d, J=5.2 Hz, 1H), 7.30-7.21 (m, 4H), 7.10 (d, J=7.6 Hz, 1H), 6.96-6.85 (m, 3H), 6.82 (d, J=8.2 Hz, 1H), 6.61 (t, J=5.2 Hz, 1H), 4.60 (d, J=6.4 Hz, 2H), 4.57 (s, 2H), 4.16 (s, 3H), 3.94 (t, J=6.4 Hz, 2H), 3.27 (t, J=6.9 Hz, 2H), 1.85-1.68 (m, 2H), 1.67-1.54 (m, 2H), 1.51-1.30 (m, 6H).

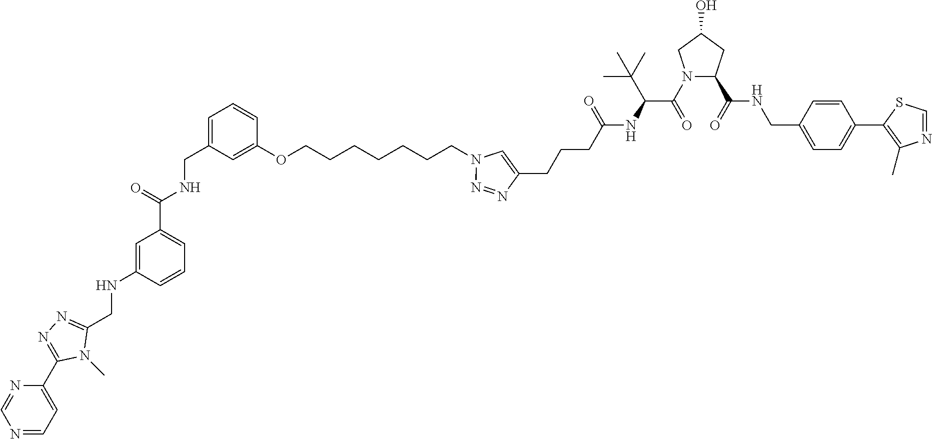 [SMILES: Cc1ncsc1-c1ccc(CNC(=O)[C@@H]2C[C@@H](O)CN2C(=O)[C@@H](NC(=O)CCCc2cn(CCCCCCCOc3cccc(CNC(=O)c4cccc(NCc5nnc(-c6ccncn6)n5C)c4)c3)nn2)C(C)(C)C)cc1]

D83. To a solution of azide 9 (0.05 g, 0.09 mmol) and alkyne 1 (0.049 g, 0.09 mmol) in ethanol (1 mL), sodium ascorbate (2 mg, 0.01 mmol) in water (0.2 mL) and copper (II) acetate (2.2 mg, 0.01 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After evaporation the product was purified by chromatography on silica, using chloroform-methanol (20 to 1, then 10 to 1) mixture as an eluent. Yield 0.09 g (91%). Colorless oil 91%. LCMS (C18 column 20×2 mm, 2.5 μm, pore size 100 Å, water-acetonitrile+0.1% TFA, gradient 5 to 87% for 10 min, retention time 6.10 min). MS (ESI) m/z 1080.1 [MH]+. ¹H NMR (400 MHz, DMSO-d₆) δ 9.33 (s, 1H), 8.98 (s, 1H), 8.96 (d, J=6.1 Hz, 1H), 8.81 (t, J=6.1 Hz, 1H), 8.55 (t, J=6.1 Hz, 1H), 8.17 (d, J=5.3 Hz, 1H), 7.89 (d, J=9.2 Hz, 1H), 7.83 (s, 1H), 7.40 (q, J=8.2 Hz, 4H), 7.24 (s, 1H), 7.20 (q, J=8.2 Hz, 2H), 7.10 (d, J=7.3 Hz, 1H), 6.90 (d, J=6.5 Hz, 1H), 6.85 (d, J=7.8 Hz, 1H), 6.83 (s, 1H), 6.77 (d, J=9.3 Hz, 1H), 6.47 (t, J=6.5 Hz, 1H), 5.12 (d, J=3.5 Hz, 1H), 4.61-4.52 (m, 3H), 4.48-4.38 (m, 4H), 4.35 (s, 1H), 4.25 (dd, J=14.7, 7.5 Hz, 2H), 4.07 (s, 3H), 3.91 (t, J=6.5 Hz, 2H), 3.66 (s, 2H), 2.57 (t, J=7.7 Hz, 2H), 2.44 (s, 3H), 2.34-2.26 (m, 1H), 2.22-2.13 (m, 1H), 2.07-1.99 (m, 1H), 1.94-1.85 (m, 1H), 1.83-1.72 (m, 4H), 1.70-1.61 (m, 2H), 1.40-1.27 (m, 4H), 1.26-1.17 (m, 2H), 0.96-0.90 (s, 10H).

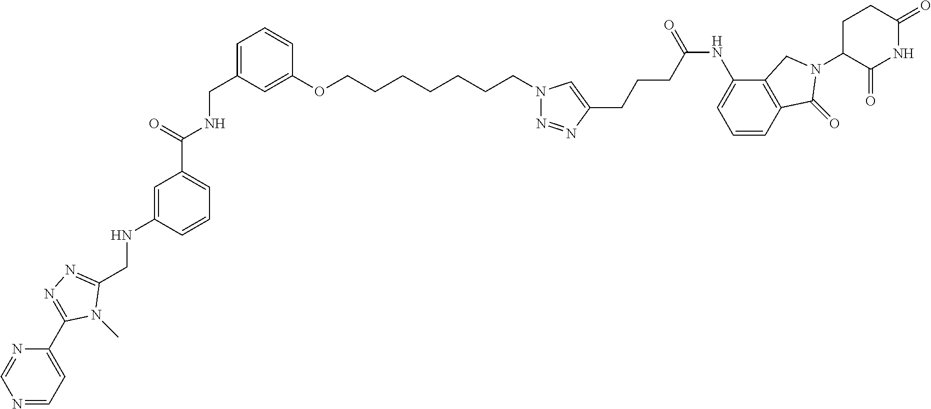 [SMILES: Cn1c(CNc2cccc(C(=O)NCc3cccc(OCCCCCCCn4cc(CCCC(=O)Nc5cccc6c5CN(C5CCC(=O)NC5=O)C6=O)nn4)c3)c2)nnc1-c1ccncn1]

D61. Colorless oil 91%. LCMS (C18 column 20×2 mm, 2.5 μm, pore size 100 Å, water-acetonitrile+0.1% TFA, gradient 5 to 87% for 10 min, retention time 5.66 min). MS (ESI) m/z 910.1 [MH]+. ¹H NMR (400 MHz, DMSO-d₆) δ 11.01 (s, 1H), 9.78 (s, 1H), 9.33 (s, 1H), 8.97 (d, J=5.2 Hz, 1H), 8.81 (t, J=6.5 Hz, 1H), 8.18 (d, J=5.2 Hz, 1H), 7.87 (s, 1H), 7.82 (d, J=6.5 Hz, 1H), 7.54-7.42 (m, 2H), 7.24 (s, 1H), 7.19 (dd, J=16.2, 8.1 Hz, 2H), 7.10 (d, J=6.6 Hz, 1H), 6.90 (d, J=7.6 Hz, 1H), 6.85 (d, J=7.1 Hz, 1H), 6.84 (s, 1H), 6.77 (d, J=9.2 Hz, 1H), 6.47 (t, J=5.2 Hz, 1H), 5.14 (dd, J=13.4, 5.1 Hz, 1H), 4.57 (d, J=5.5 Hz, 2H), 4.40 (d, J=6.2 Hz, 2H), 4.36 (d, J=8.9 Hz, 2H), 4.28 (dd, J=14.3, 7.2 Hz, 2H), 4.07 (s, 3H), 3.91 (t, J=6.4 Hz, 2H), 2.96-2.87 (m, 1H), 2.67 (t, J=7.6 Hz, 2H), 2.60 (d, J=16.1 Hz, 1H), 2.41 (t, J=7.4 Hz, 2H), 2.34 (dd, J=16.1 Hz, 7.4 Hz, 1H), 2.06-1.98 (m, 1H), 1.97-1.86 (m, 2H), 1.84-1.73 (m, 2H), 1.69-1.62 (m, 2H), 1.40-1.28 (m, 4H), 1.26-1.16 (m, 2H).

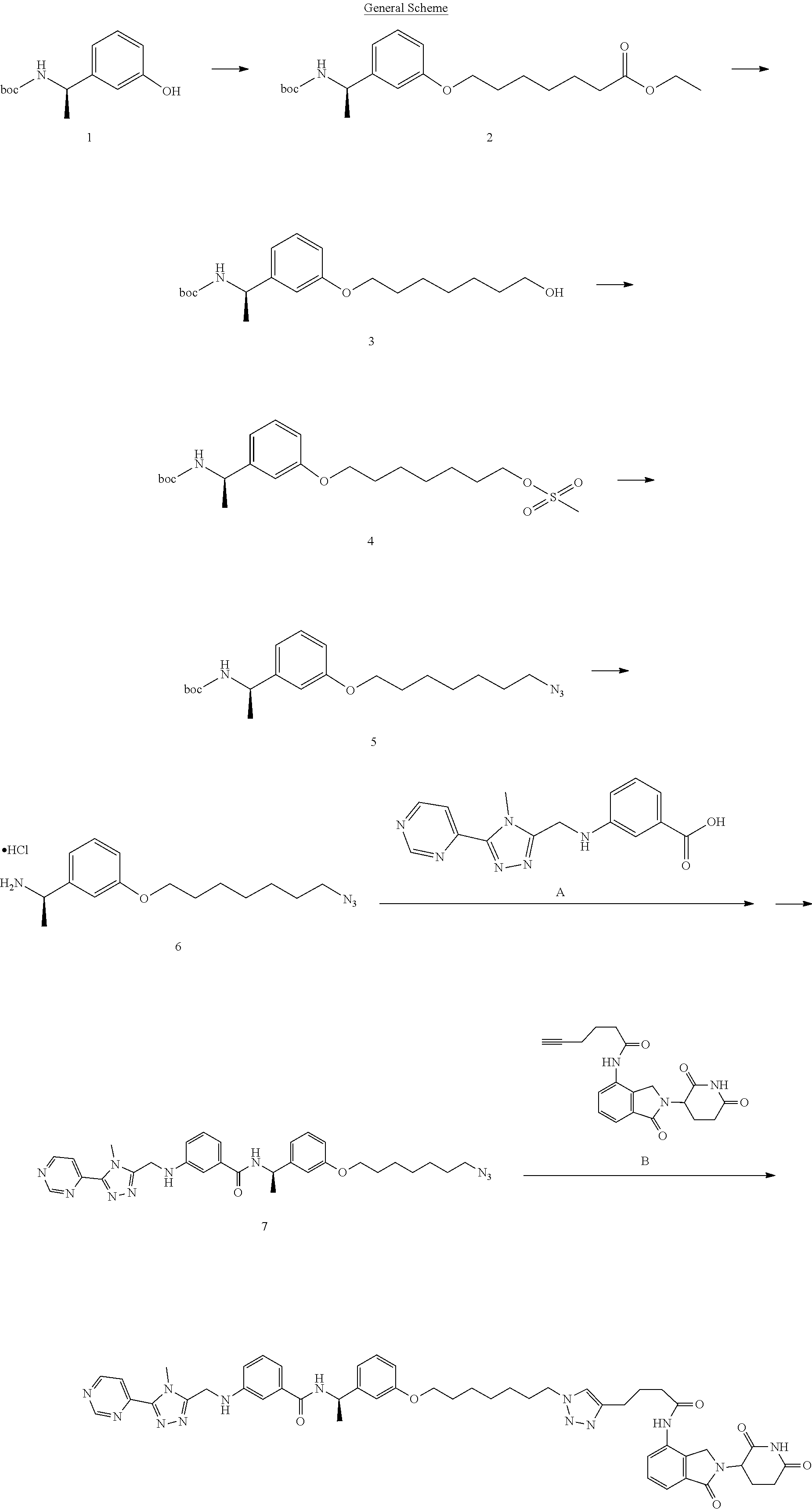

### Compounds, Procedures, and Analytical Data

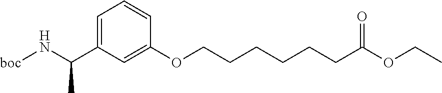

Compound 2. (R)-ethyl 7-(3-(1-(tert-butoxycarbonylamino)ethyl)phenoxy)heptanoate. A mixture of compound 1 (1000 mg, 4.2 mmol), ethyl bromoheptanoate (1100 mg, 4.6 mmol), K₂CO₃ (1.16 g, 2.53 mmol) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 1150 mg (69%) of the desired product 2. ¹H NMR (400 MHz, DMSO-d₆) δ 7.30 (d, J=4.5 Hz, 1H), 7.18 (t, J=7.9 Hz, 1H), 6.84-6.82 (m, 2H), 6.75 (dd, J=8.0, 1.6 Hz, 1H), 4.59-4.52 (m, 1H), 4.07-4.02 (m, 2H), 3.92 (t, J=6.5 Hz, 2H), 1.73-1.67 (m, 2H), 1.42-1.26 (m, 20H), 1.17 (t, 3H).

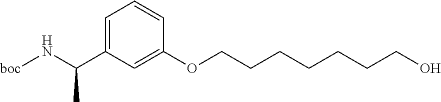

Compound 3. (R)-tert-butyl 1-(3-(7-hydroxyheptyloxy)phenyl)ethylcarbamate. To a mixture of compound 2 (1150 mg, 29.3 mmol) in Et₂O (5 mL), LiBH₄ (96 mg, 43.9 mmol) was added, followed by methanol (140 mg, 43.9 mmol) at 0° C. The reaction mixture was stirred at ambient temperature overnight. The solvent was evaporated and the residue was partitioned between DCM (5 ml) and water, contains with citric acid (2 g in water 10 ml). The extract was separated and dried over Na₂SO₄. The crude product 3 was used further without additional purification. Yield (616 mg, 60%). Colorless oil LCMS (C18 column 20×2 mm, 2.5 μm, pore size 100 Å, water-acetonitrile+0.1% TFA, gradient 5 to 87% for 3 min, retention time 1.73 min). MS (ESI) m/z 352.5 [MH]+. ¹H NMR (400 MHz, DMSO-d₆) δ 7.32 (d, J=4.5 Hz, 1H), 7.18 (t, J=7.9 Hz, 1H), 6.84-6.82 (m, 2H), 6.76 (dd, J=8.0, 1.6 Hz, 1H), 4.59-4.52 (m, 1H), 4.32 (t, J=5.1 Hz, 1H), 3.92 (t, J=6.5 Hz, 2H), 3.40-3.36 (m, 2H), 1.73-1.67 (m, 2H), 1.43-1.26 (m, 20H)

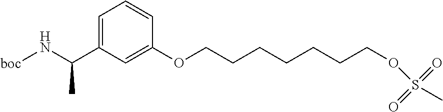

Compound 4. (R)-7-(3-(1-(tert-butoxycarbonylamino)ethyl)phenoxy)heptyl methanesulfonate A solution of compound 3 (600 mg, 1.7 mmol) in CH₂Cl₂ was mixed with 0.444 mL of DIPEA (2.55 mmol) and cooled to 0° C. 234 mg of MsCl (2.05 mmol) was added dropwise. The reaction mixture was stirred at room temperature for 2 hours, washed with water, dried over Na₂SO₄ and evaporated. The product was used without purification. Yield 720 mg (98%)

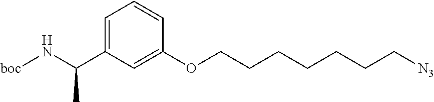

Compound 5. (R)-tert-butyl 1-(3-(7-azidoheptyloxy)phenyl)ethylcarbamate. To a solution of compound 4 (728 mg, 1.7 mmol) in dried DMF (10 ml) NaN₃ (0.166 g, 2.55 mmol) was added and the reaction mixture was stirred at ambient temperature overnight. Then the resulted slurry was poured onto water (100 ml) and the product was extracted with ethyl acetate (3 times by 20 ml). Combined organic layers were washed with water (3 times by 20 ml) and then once with brine (10 ml). After drying over MgSO4 the solvent was stripped off and product was used further without purification. Yield 619 mg (97%). LCMS (C18 column 20×2 mm, 2.5 μm, pore size 100 Å, water-acetonitrile+0.1% TFA, gradient 5 to 87% for 3 min, retention time 2.19 min). MS (ESI) m/z 377.6 [MH]+. ¹H NMR (400 MHz, CDCl₃) δ 7.32 (d, J=4.5 Hz, 1H), 7.18 (t, J=7.8 Hz, 1H), 6.84-6.82 (m, 2H), 6.76 (dd, J=8.0, 1.6 Hz, 1H), 4.58-4.51 (m, 1H), 3.93 (t, J=6.4 Hz, 2H), 3.31 (t, J=6.8 Hz, 2H), 1.73-1.64 (m, 2H), 1.55-1.50 (m, 2H), 1.41-1.23 (m, 18H).

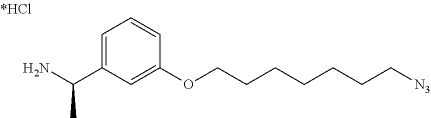 [SMILES: C[C@@H](N)c1cccc(OCCCCCCCN=[N+]=[N-])c1]

Compound 6. (R)-1-(3-(7-azidoheptyloxy)phenyl)ethanamine hydrochloride. To an ice cold solution of azide 5 (0.619 g, 1.64 mmol) in methylene chloride (20 ml) a dioxanic 3M HCl solution (3.3 ml) was added. The mixture was stirred at ambient temperature overnight and then evaporated dryness. The residue was taken up with dry ether (20 ml) and the solid was filtered, washed with ether (10 ml) and dried on air. Yield 530 mg (98%). LCMS (C18 column 20×2 mm, 2.5 μm, pore size 100 Å, water-acetonitrile+0.1% TFA, gradient 5 to 87% for 3 min, retention time 1.25 min). MS (ESI) m/z 277.5 [MH]+. ¹H NMR (400 MHz, DMSO-d₆) δ 8.58 (s, 3H), 7.30 (t, J=7.9 Hz, 1H), 7.15 (s, 1H), 7.04 (d, J=7.4 Hz, 1H), 6.91 (d, J=8.2 Hz, 1H), 4.35-4.30 (m, 1H), 3.98 (t, J=6.2 Hz, 2H), 3.32 (t, J=6.2 Hz, 2H), 1.74-1.70 (m, 2H), 1.59-1.49 (m, 3H), 1.41-1.35 (m, 6H).

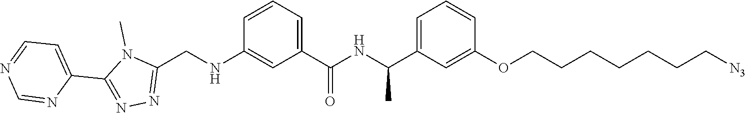 [SMILES: C[C@@H](NC(=O)c1cccc(NCc2nnc(-c3ccncn3)n2C)c1)c1cccc(OCCCCCCCN=[N+]=[N-])c1]

Compound 7. (R)—N-(1-(3-(7-azidoheptyloxy)phenyl)ethyl)-3-((4-methyl-5-(pyrimidin-4-yl)-4H-1,2,4-triazol-3-yl)methylamino)benzamide. To an ice cold suspension of amine hydrochloride 6 (0.250 g, 0.8 mmol) in methylene chloride (5 ml) acid A (0.273 g, 0.88 mmol) was added, followed by hydroxyl benzotriazole hydrate (0.146 g, 0.96 mmol) and EDCI (0.149 g, 0.96 mmol) and finally DIPEA (0.53 ml, 3.6 mmol). The reaction mixture was stirred at ambient temperature overnight. Then water (5 ml) was added and organic layer was separated. Aqueous layer was extracted with methylene chloride (5 ml). Combined organic layers were dried over MgSO₄ and evaporated. The residue was purified by chromatography on silica, using chloroform-methanol (20 to 1) mixture as an eluent. Yield 0.373 g (82%). Colorless oil 50%. LCMS (C18 column 20×2 mm, 2.5 μm, pore size 100 Å, water-acetonitrile+0.1% TFA, gradient 5 to 87% for 3 min, retention time 1.73 min). MS (ESI) m/z 569.7[MH]+. ¹H NMR (400 MHz, CDCl₃) δ 9.33 (s, 1H), 8.86 (d, J=5.2 Hz, 1H), 8.55 (d, 1H), 8.18 (d, 1H), 7.22-7.16 (m, 3H), 7.11 (d, J=7.6 Hz, 1H), 6.93-6.89 (m, 3H), 6.75 (d, J=8.2 Hz, 1H), 6.46 (t, 1H), 5.13-5.07 (m, 1H), 4.58 (d, 2H), 4.07 (s, 3H), 3.93 (t, J=6.3 Hz, 2H), 3.30 (t, J=6.9 Hz, 2H), 1.72-1.65 (m, 2H), 1.58-1.51 (m, 2H), 1.43-1.32 (m, 9H).

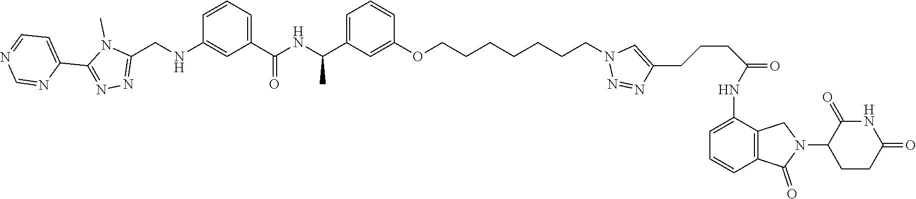 [SMILES: C[C@@H](NC(=O)c1cccc(NCc2nnc(-c3ccncn3)n2C)c1)c1cccc(OCCCCCCCn2cc(CCCC(=O)Nc3cccc4c3CN(C3CCC(=O)NC3=O)C4=O)nn2)c1]

D158. N-((1R)-1-(3-(7-(4-(4-(2-(2,6-dioxopiperidin-3-yl)-1-oxoisoindolin-4-ylamino)-4-oxobutyl)-1H-1,2,3-triazol-1-yl)heptyloxy)phenyl)ethyl)-3-((4-methyl-5-(pyrimidin-4-yl)-4H-1,2,4-triazol-3-yl)methylamino)benzamide. To a solution of azide 7 (0.10 g, 0.35 mmol) and alkyne B (0.058 g, 0.35 mmol) in ethanol (1 ml), sodium ascorbate (3.5 mg, 0.035 mmol) in water (0.2 ml) and copper (II) acetate (3.2 mg, 0.035 mmol) in water (0.2 ml) were added successively. The mixture was stirred at ambient temperature overnight. Then the solvent was stripped off and the residue was partitioned between methylene chloride (5 ml) and water (5 ml). Organic phase was separated and dried over MgSO₄. The residue was purified by HPLC in neutral conditions (YMC-Pack ODS-AQ 250×20 mml, S-10 μm, 12 nm, gradient water-acetonitrile). Yield 0.011 g (7%). LCMS (C18 column 20×2 mm, 2.5 μm, pore size 100 Å, water-acetonitrile+0.1% TFA, gradient 5 to 87% for 10 min, retention time 5.74 min). MS (ESI) m/z 923.1 [MH]+. ¹H NMR (400 MHz, DMSO-d₆) δ 11.00 (s, 1H), 9.78 (s, 1H), 9.33 (s, 1H), 8.96 (d, J=5.3 Hz, 1H), 8.54 (d, J=8.2 Hz, 1H), 8.16 (dd, J=5.3, 1.4 Hz, 1H), 7.87 (s, 1H), 7.82 (d, J=7.0 Hz, 1H), 7.51-7.45 (m, 2H), 7.21-7.15 (m, 3H), 7.11 (d, J=8.1 Hz, 1H), 6.93-6.88 (m, 3H), 6.76 (d, J=9.3 Hz, 1H), 6.46 (t, J=5.3 Hz, 1H), 5.17-5.07 (m, 2H), 4.56 (d, J=5.4 Hz, 2H), 4.42-4.30 (m, 2H), 4.27 (t, J=5.8 Hz, 2H), 4.06 (s, 3H), 3.91 (t, J=6.4 Hz, 2H), 2.96-2.88 (m, 1H), 2.67 (t, J=7.5 Hz, 2H), 2.63-2.57 (m, 1H), 2.41 (t, J=7.5 Hz, 2H), 2.37-2.30 (m, 1H), 2.05-1.99 (m, 1H), 1.96-1.89 (m, 2H), 1.81-1.74 (m, 2H), 1.70-1.63 (m, 2H), 1.45-1.20 (m, 9H).

### Example 19: Preparation of Compounds D82 and D60

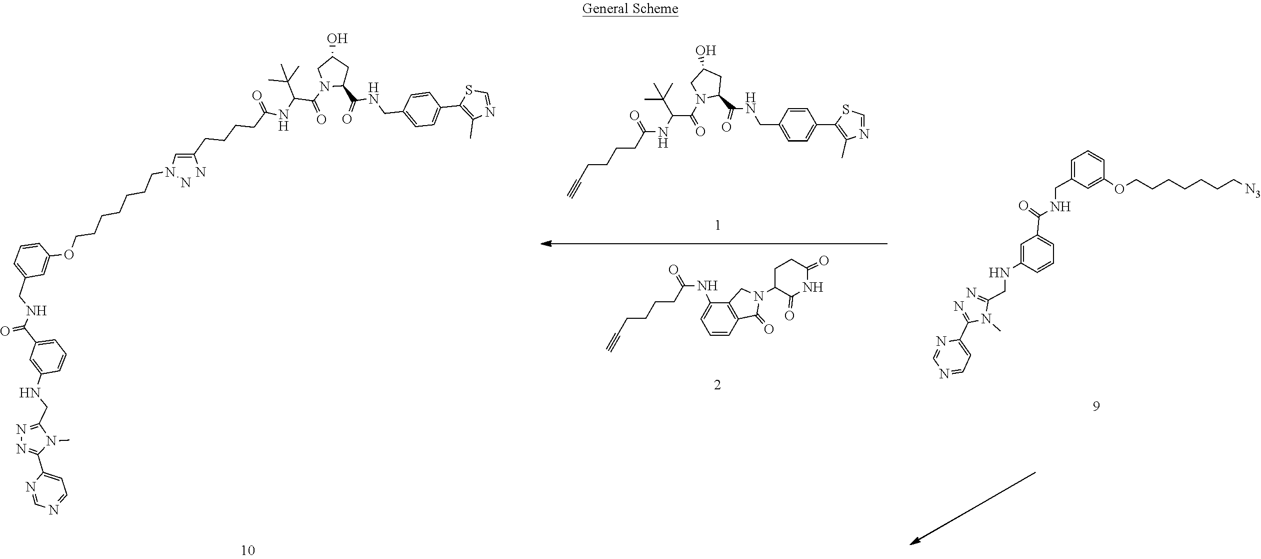 [SMILES: Cc1ncsc1-c1ccc(CNC(=O)[C@@H]2C[C@@H](O)CN2C(=O)C(NC(=O)CCCCc2cn(CCCCCCCOc3cccc(CNC(=O)c4cccc(NCc5nnc(-c6ccncn6)n5C)c4)c3)nn2)C(C)(C)C)cc1 ; C#CCCCCC(=O)NC(C(=O)N1C[C@H](O)C[C@H]1C(=O)NCc1ccc(-c2scnc2C)cc1)C(C)(C)C ; C#CCCCCC(=O)Nc1cccc2c1CN(C1CCC(=O)NC1=O)C2=O ; Cn1c(CNc2cccc(C(=O)NCc3cccc(OCCCCCCCN=[N+]=[N-])c3)c2)nnc1-c1ccncn1]

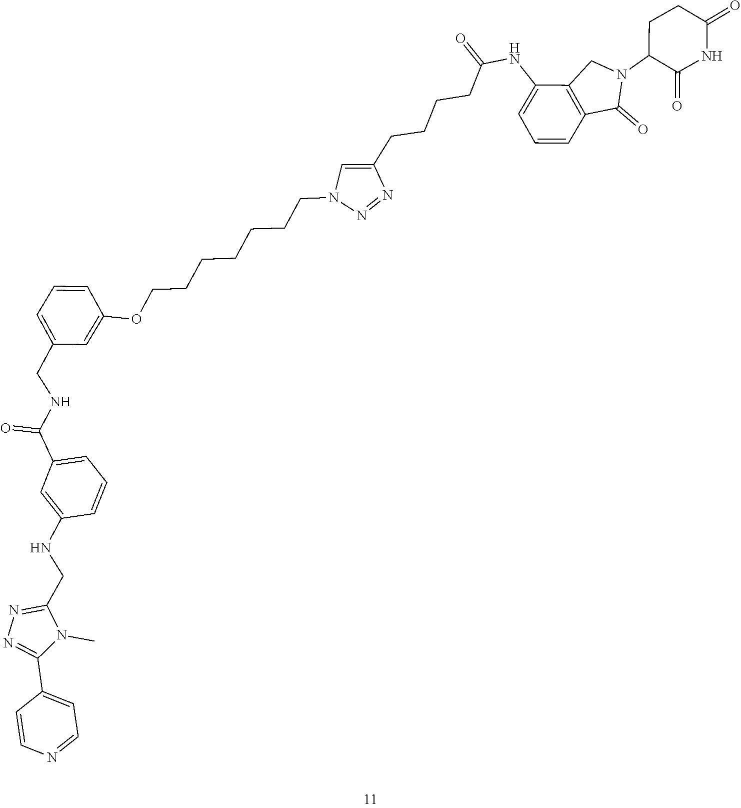 [SMILES: Cn1c(CNc2cccc(C(=O)NCc3cccc(OCCCCCCCn4cc(CCCCC(=O)Nc5cccc6c5CN(C5CCC(=O)NC5=O)C6=O)nn4)c3)c2)nnc1-c1ccncc1]

### Compounds, Procedure, and Analytical Data

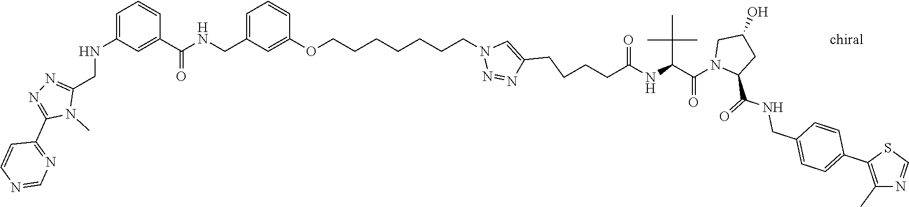 [SMILES: Cc1ncsc1-c1ccc(CNC(=O)[C@@H]2C[C@@H](O)CN2C(=O)[C@@H](NC(=O)CCCCc2cn(CCCCCCCOc3cccc(CNC(=O)c4cccc(NCc5nnc(-c6ccncn6)n5C)c4)c3)nn2)C(C)(C)C)cc1]

D82. Colorless oil 91%. LCMS (C18 column 20×2 mm, 2.5 μm, pore size 100 Å, water-acetonitrile+0.1% TFA, gradient 5 to 87% for 10 min, retention time 6.17 min). MS (ESI) m/z 1095.2 [MH]+. ¹H NMR (400 MHz, DMSO-d₆) δ 9.33 (s, 1H), 9.02-8.92 (m, 2H), 8.81 (t, 1H), 8.55 (t, J=5.1 Hz, 1H), 8.17 (d, J=5.2 Hz, 1H), 7.85 (d, J=9.3 Hz, 1H), 7.80 (s, 1H), 7.39 (q, J=8.3 Hz, 4H), 7.27-7.13 (m, 3H), 7.10 (d, J=7.5 Hz, 1H), 6.90 (d, J=6.2 Hz, 1H), 6.87-6.81 (m, 2H), 6.77 (d, J=9.3 Hz, 1H), 6.47 (t, J=5.5 Hz, 1H), 5.12 (d, J=3.6 Hz, 1H), 4.63-4.50 (m, 3H), 4.49-4.38 (m, 4H), 4.34 (s, 1H), 4.31-4.16 (m, 3H), 4.07 (s, 3H), 3.91 (t, J=6.5 Hz, 2H), 3.65 (s, 2H), 2.58 (t, J=5.0 Hz, 2H), 2.44 (s, 3H), 2.35-2.22 (m, 1H), 2.21-2.10 (m, 1H), 2.07-1.98 (m, 1H), 1.95-1.85 (m, 1H), 1.83-1.72 (m, 2H), 1.71-1.61 (m, 2H), 1.61-1.45 (m, 4H), 1.42-1.26 (m, 4H), 1.26-1.14 (m, 2H), 0.92 (s, 9H).

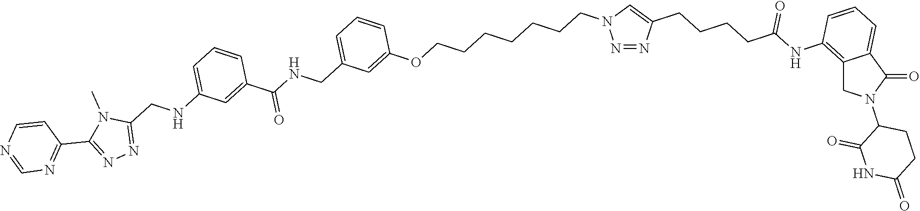 [SMILES: Cn1c(CNc2cccc(C(=O)NCc3cccc(OCCCCCCCn4cc(CCCCC(=O)Nc5cccc6c5CN(C5CCC(=O)NC5=O)C6=O)nn4)c3)c2)nnc1-c1ccncn1]

D60. Colorless oil 91%. LCMS (C18 column 20×2 mm, 2.5 μm, pore size 100 Å, water-acetonitrile+0.1% TFA, gradient 5 to 87% for 10 min, retention time 5.71 min). MS (ESI) m/z 923.2 [MH]+. ¹H NMR (400 MHz, DMSO-d₆) δ 11.00 (s, 1H), 9.76 (s, 1H), 9.33 (s, 1H), 8.97 (d, J=5.3 Hz, 1H), 8.80 (t, J=5.5 Hz, 1H), 8.17 (dd, J=5.2, 1.4 Hz, 1H), 7.88-7.75 (m, 2H), 7.54-7.43 (m, 2H), 7.28-7.14 (m, 3H), 7.10 (d, J=7.8 Hz, 1H), 6.90 (d, J=6.3 Hz, 1H), 6.88-6.80 (m, 2H), 6.77 (d, J=9.1 Hz, 1H), 6.46 (t, J=5.2 Hz, 1H), 5.14 (dd, J=13.3, 5.0 Hz, 1H), 4.57 (d, J=5.8 Hz, 2H), 4.40 (d, J=5.6 Hz, 2H), 4.36 (d, J=7.0 Hz, 2H), 4.26 (t, J=7.0 Hz, 2H), 4.07 (s, 3H), 3.90 (t, J=6.4 Hz, 2H), 3.00-2.83 (m, 1H), 2.70-2.56 (m, J=20.8 Hz, 3H), 2.44-2.29 (m, J=15.9 Hz, 3H), 2.08-1.96 (m, 1H), 1.85-1.72 (m, 2H), 1.72-1.57 (m, 6H), 1.44-1.12 (m, 6H).

### Example 20: Preparation of Compound D102

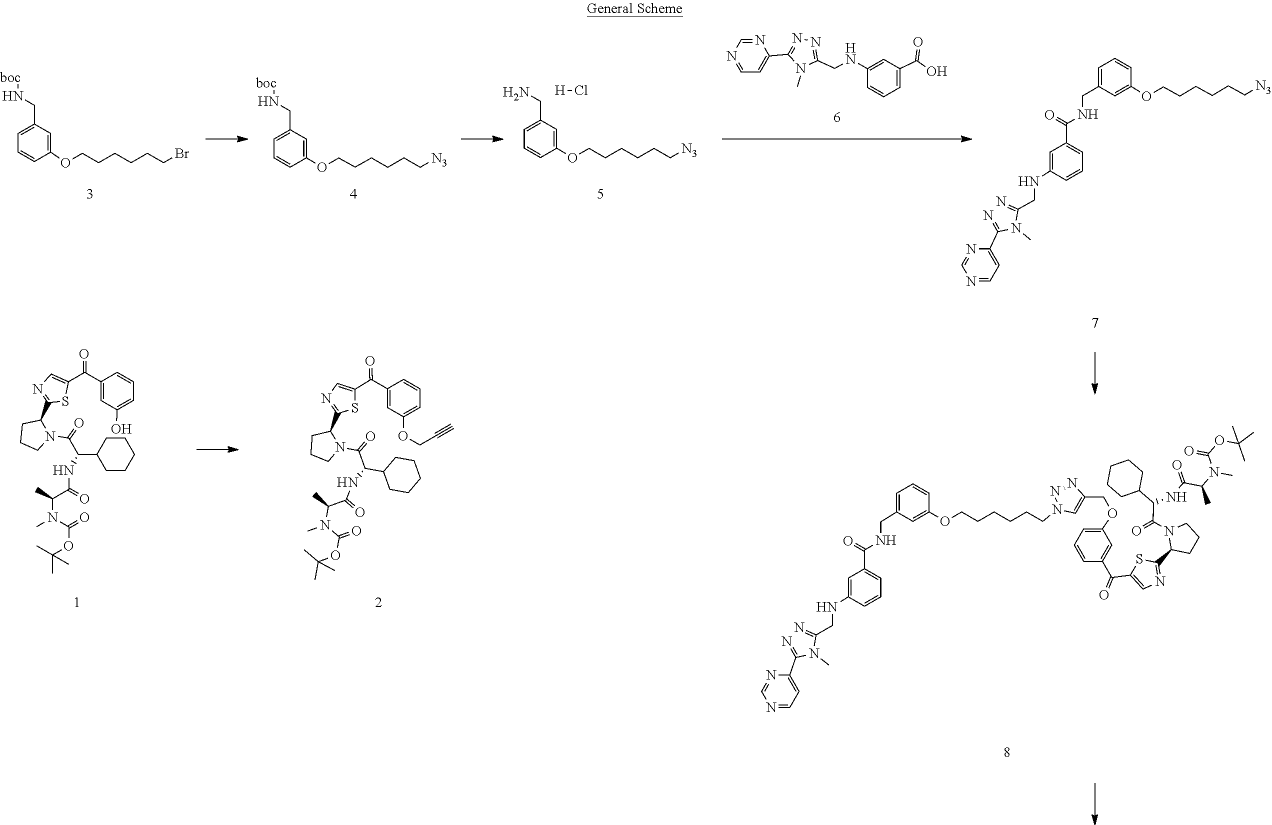

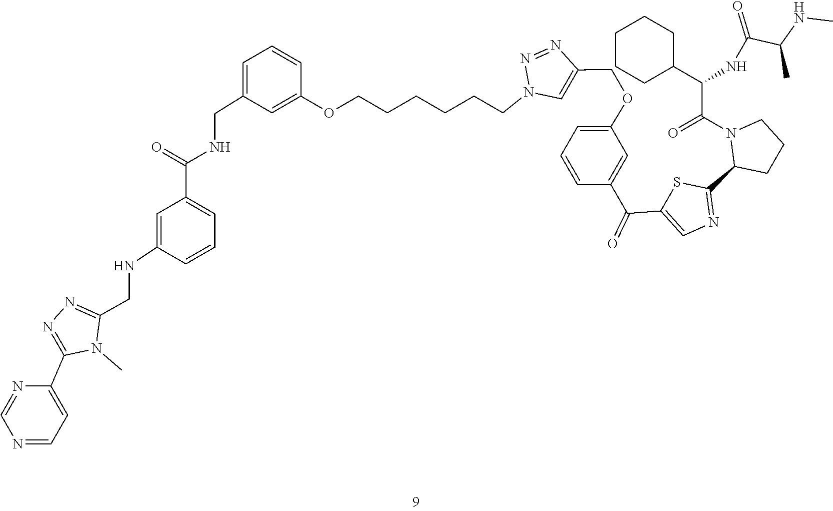 [SMILES: CN[C@@H](C)C(=O)N[C@H](C(=O)N1CCC[C@H]1c1ncc(C(=O)c2cccc(OCc3cn(CCCCCCOc4cccc(CNC(=O)c5cccc(NCc6nnc(-c7ccncn7)n6C)c5)c4)nn3)c2)s1)C1CCCCC1]

### Compounds, Procedures, Analytical Data

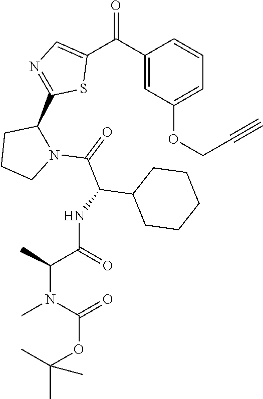 [SMILES: C#CCOc1cccc(C(=O)c2cnc([C@@H]3CCCN3C(=O)[C@@H](NC(=O)[C@H](C)N(C)C(=O)OC(C)(C)C)C3CCCCC3)s2)c1]

To a solution of phenol 1 (0.7 g, 11.7 mmol) in dried DMF (5 mL), a solution of 70% propargyl bromide in toluene (0.15 mL, 13 mmol) was added dropwise, followed by K₂CO₃ (0.2 g, 14.5 mmol). The reaction mixture was stirred at ambient temperature overnight and then evaporated at reduced pressure dryness. The residue was partitioned between methylene chloride (5 mL) and water (5 mL). Organic phase was separated. Aqueous phase was extracted with methylene chloride (5 mL). Combined organic phases were washed with water (5 mL) and dried over MgSO₄. After the solvent evaporation the crude product was purified on silica, using chloroform-methanol (100 to 1, then 60 to 1) mixture as an eluent. Yield 0.7 g (94%). Colorless oil 94%. LCMS (C18 column 20×2 mm, 2.5 μm, pore size 100 Å, water-acetonitrile+0.1% TFA, gradient 5 to 87% for 3 min, retention time 2.26 min). MS (ESI) m/z 637.4 [MH]+.

¹H NMR (400 MHz, CDCl₃) δ 8.14 (s, 1H), 8.03 (s, 1H), 7.88-7.2 (m, 2H), 7.42 (t, J=7.9 Hz, 1H), 7.23 (d, J=6.4 Hz, 1H), 5.57 (d, J=5.6 Hz, 1H), 4.77 (d, J=2.3 Hz, 2H), 4.69-4.49 (m, 1H), 3.93-3.87 (m, 1H), 3.84-3.78 (m, 1H), 2.81 (s, 3H), 2.55 (t, J=2.3 Hz, 1H), 2.56-2.46 (m, 1H), 2.35-2.11 (m, 3H), 1.78-1.61 (m, 9H), 1.48 (s, 9H), 1.35 (d, J=7.1 Hz, 2H), 1.20-0.98 (m, 5H).

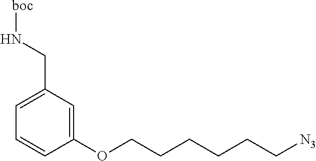

To a solution of bromide 3 (5.3 g, 13.7 mmol) in dried DMF (10 mL) NaN₃ (1.8 g, 27.7 mmol) was added and the reaction mixture was stirred at ambient temperature overnight. Then the resulted slurry was poured onto water (100 mL) and the product was extracted with ethyl acetate (3 times by 20 mL). Combined organic layers were washed with water (3 times by 20 mL) and then once with brine (10 mL). After drying over MgSO₄ the solvent was stripped off and product was used further without purification. Yield 2.4 g (50%). Colorless oil 50%. LCMS (C18 column 20×2 mm, 2.5 μm, pore size 100 Å, water-acetonitrile+0.1% TFA, gradient 5 to 87% for 3 min, retention time 2.32 min). MS (ESI) m/z 349.6 [MH]+. ¹H NMR (400 MHz, CDCl₃) δ 7.24 (t, J=7.8 Hz, 1H), 6.90-6.75 (m, 3H), 4.84 (s, 1H), 4.29 (d, J=4.7 Hz, 2H), 3.96 (t, J=6.4 Hz, 2H), 3.30 (t, J=6.8 Hz, 2H), 1.80 (dd, J=14.1, 6.6 Hz, 2H), 1.66 (dd, J=14.2, 7.0 Hz, 2H), 1.56-1.44 (m, 13H).

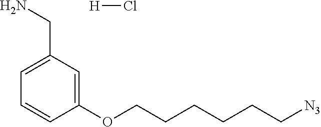 [SMILES: [N-]=[N+]=NCCCCCCOc1cccc(CN)c1 ; Cl]

To an ice cold solution of azide 4 (2.2 g, 6.32 mmol) in methylene chloride (20 mL) a dioxanic 3M HCl solution (10 mL) was added. The mixture was stirred at ambient temperature overnight and then evaporated dryness. The residue was taken up with dry ether (20 mL) and the solid was filtered, washed with ether (10 mL) and dried on air. Yield 1.77 g (98%).

Colorless solid 98%. ¹H NMR (400 MHz, DMSO-d₆) δ 8.47 (s, 3H), 7.30 (t, J=7.9 Hz, 1H), 7.12 (s, 1H), 7.02 (d, J=7.4 Hz, 1H), 6.92 (d, J=8.2 Hz, 1H), 3.97 (t, J=6.2 Hz, 4H), 1.80-1.64 (m, 2H), 1.57 (dd, J=14.0, 6.9 Hz, 2H), 1.41 (t, J=10.9 Hz, 4H).

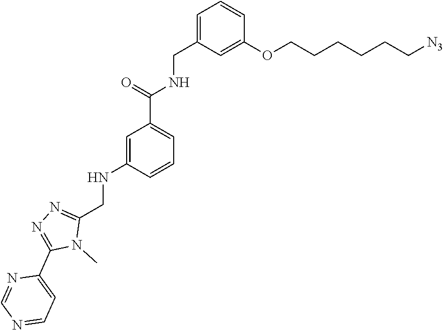 [SMILES: Cn1c(CNc2cccc(C(=O)NCc3cccc(OCCCCCCN=[N+]=[N-])c3)c2)nnc1-c1ccncn1]

To an ice cold suspension of amine hydrochloride 5 (0.46 g, 1.6 mmol) in methylene chloride (5 mL), acid 6 (0.5 g, 1.6 mmol) was added, followed by hydroxyl benzotriazole hydrate (0.27 g, 1.7 mmol) and EDCI (0.34 g, 1.8 mmol) and finally DIPEA (0.85 mL, 0.63 g, 4.9 mmol). The reaction mixture was stirred at ambient temperature overnight. Then water (5 mL) was added and organic layer was separated. Aqueous layer was extracted with methylene chloride (5 mL). Combined organic layers were dried over MgSO₄ and evaporated. The residue was purified by chromatography on silica, using chloroform-methanol (20 to 1) mixture as an eluent. Yield 0.79 g (90%). Colorless oil 50%. LCMS (C18 column 20×2 mm, 2.5 μm, pore size 100 Å, water-acetonitrile+0.1% TFA, gradient 5 to 87% for 3 min, retention time 1.59 min). MS (ESI) m/z 541.7 [MH]+. ¹H NMR (400 MHz, CDCl₃) δ 9.27 (s, 1H), 8.86 (d, J=5.2 Hz, 1H), 8.25 (d, J=5.2 Hz, 1H), 7.36-7.17 (m, 4H), 7.10 (d, J=7.6 Hz, 1H), 6.97-6.86 (m, 3H), 6.82 (d, J=8.2 Hz, 1H), 6.61 (s, 1H), 4.68-4.48 (m, 4H), 4.16 (s, 3H), 3.95 (t, J=6.3 Hz, 2H), 3.28 (t, J=6.9 Hz, 2H), 1.87-1.70 (m, 2H), 1.68-1.55 (m, 2H), 1.55-1.35 (m, 4H).

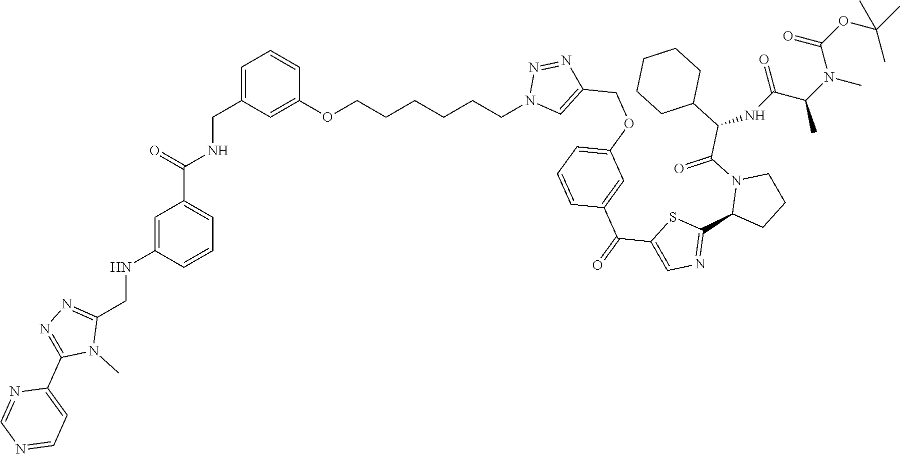 [SMILES: C[C@@H](C(=O)N[C@H](C(=O)N1CCC[C@H]1c1ncc(C(=O)c2cccc(OCc3cn(CCCCCCOc4cccc(CNC(=O)c5cccc(NCc6nnc(-c7ccncn7)n6C)c5)c4)nn3)c2)s1)C1CCCCC1)N(C)C(=O)OC(C)(C)C]

To a solution of azide 7 (0.1 g, 0.18 mmol) and alkyne 2 (0.12 g, 0.19 mmol) in ethanol (1 mL), sodium ascorbate (4 mg, 0.02 mmol) in water (0.2 mL) and copper (II) acetate (4.4 mg, 0.02 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After the solvent evaporation the product was purified by chromatography on silica, using chloroform-methanol (20 to 1, then 10 to 1) mixture as an eluent. Yield 0.21 g (96%). Colorless oil 96%. LCMS (C18 column 20×2 mm, 2.5 μm, pore size 100 Å, water-acetonitrile+0.1% TFA, gradient 5 to 87% for 3 min, retention time 1.8 min). MS (ESI) m/z 1178.1 [MH]+.

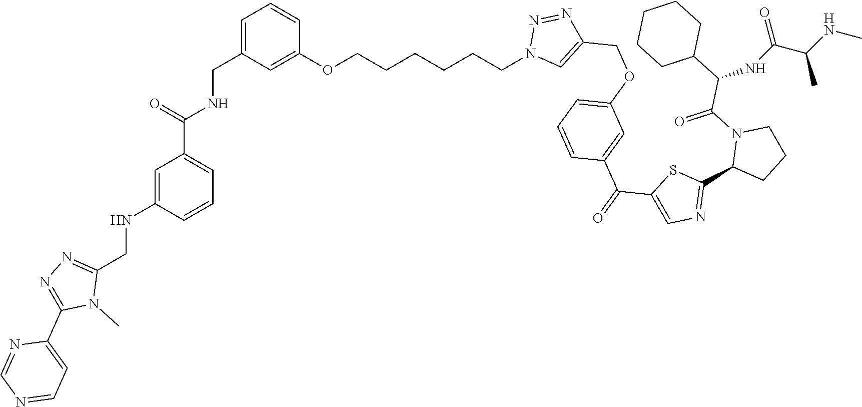 [SMILES: CN[C@@H](C)C(=O)N[C@H](C(=O)N1CCC[C@H]1c1ncc(C(=O)c2cccc(OCc3cn(CCCCCCOc4cccc(CNC(=O)c5cccc(NCc6nnc(-c7ccncn7)n6C)c5)c4)nn3)c2)s1)C1CCCCC1]

D102. N-(3-((6-(4-((3-(2-((S)-1-((S)-2-cyclohexyl-2-((S)-2-(methylamino)propanamido) acetyl)pyrrolidin-2-yl)thiazole-4-carbonyl)phenoxy)methyl)-1H-1,2,3-triazol-1-yl)hexyl)oxy)benzyl)-3-(((4-methyl-5-(pyrimidin-4-yl)-4H-1,2,4-triazol-3-yl)methyl)amino)benzamide To an ice cold solution of triazole 8 (0.21 g, 0.17 mmol) in methylene chloride (2 mL) a dioxanic 3M HCl solution (2 mL) was added. The mixture was stirred at ambient temperature overnight and then evaporated dryness. The residue was partitioned between methylene chloride (2 mL) and aqueous NaHCO₃ (10 mL). Organic phase was separated, dried over MgSO₄ and concentrated. Product was purified by chromatography, using chloroform-methanol (20 to 1, then 10 to 1) mixture as an eluent. Yield 0.14 g (72%). Colorless oil 72%. LCMS (C18 column 20×2 mm, 2.5 μm, pore size 100 Å, water-acetonitrile+0.1% TFA, gradient 5 to 87% for 10 min, retention time 5.33 min). MS (ESI) m/z 1078.5 [MH]+. ¹H NMR (400 MHz, DMSO-d₆) δ 9.32 (s, 1H), 8.96 (d, J=5.3 Hz, 1H), 8.81 (t, J=5.3 Hz, 1H), 8.47 (s, 1H), 8.23 (s, 1H), 8.17 (d, J=5.3 Hz, 1H), 7.89 (d, J=9.2 Hz, 1H), 7.73 (s, 1H), 7.47 (t, J=7.9 Hz, 1H), 7.33 (d, J=10.9 Hz, 1H), 7.24 (s, 1H), 7.19 (dd, J=17.1, 8.2 Hz, 2H), 7.10 (d, J=7.6 Hz, 1H), 6.90 (d, J=7.7 Hz, 1H), 6.85 (d, J=8.0 Hz, 2H), 6.77 (d, J=9.2 Hz, 1H), 6.47 (t, J=7.6 Hz, 1H), 5.75 (s, 1H), 5.38 (d, J=8.4 Hz, 1H), 5.21 (s, 2H), 4.57 (d, J=5.6 Hz, 2H), 4.48 (t, J=5.6 Hz, 1H), 4.40 (d, J=6.0 Hz, 2H), 4.35 (t, J=7.0 Hz, 2H), 4.07 (s, 3H), 3.90 (t, J=6.3 Hz, 2H), 3.78 (t, J=7.2 Hz, 2H), 2.96 (m, 1H), 2.30-2.22 (m, 1H), 2.17 (d, J=6.0 Hz, 4H), 2.08-1.93 (m, 4H), 1.86-1.78 (m, 2H), 1.70-1.57 (m, 5H), 1.56-1.51 (m, 2H), 1.44-1.36 (m, 2H), 1.30-1.22 (m, 2H), 1.14-0.90 (m, 8H).

### Example 21: Preparation of Compound D103

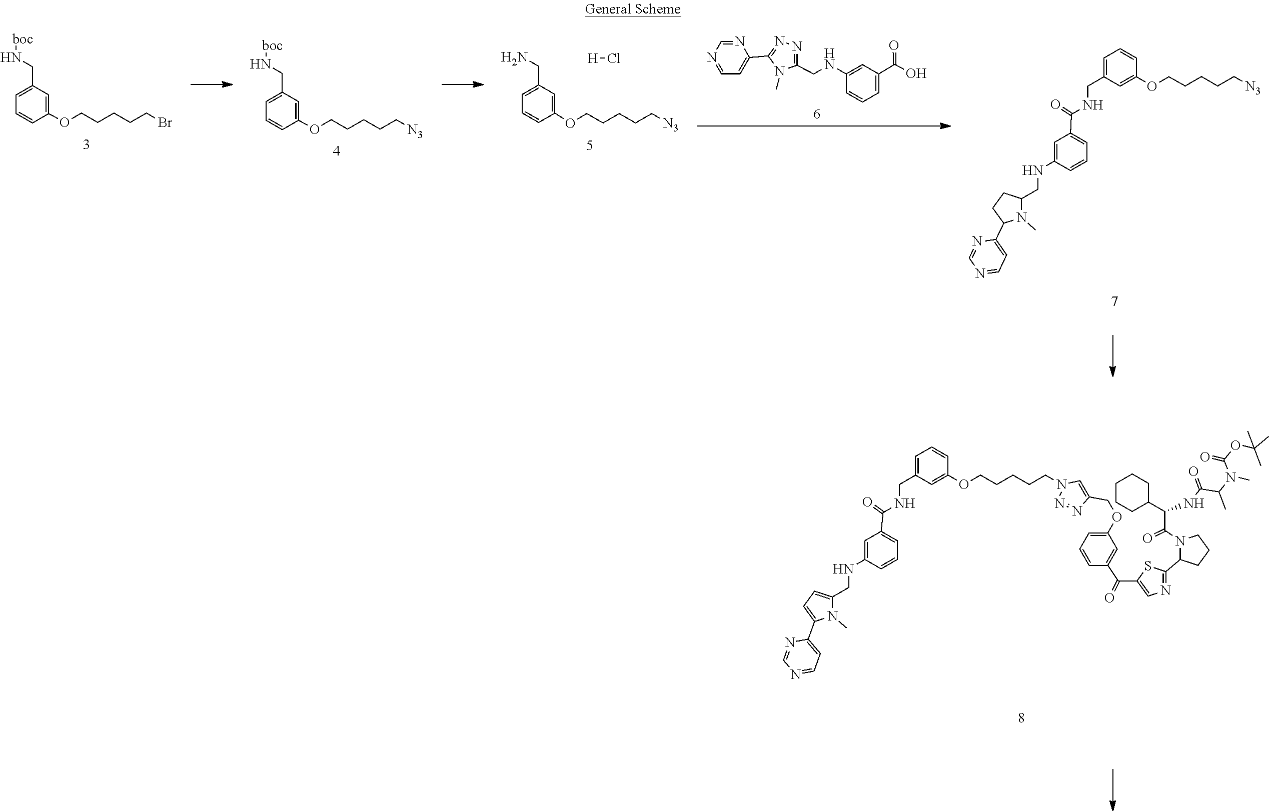

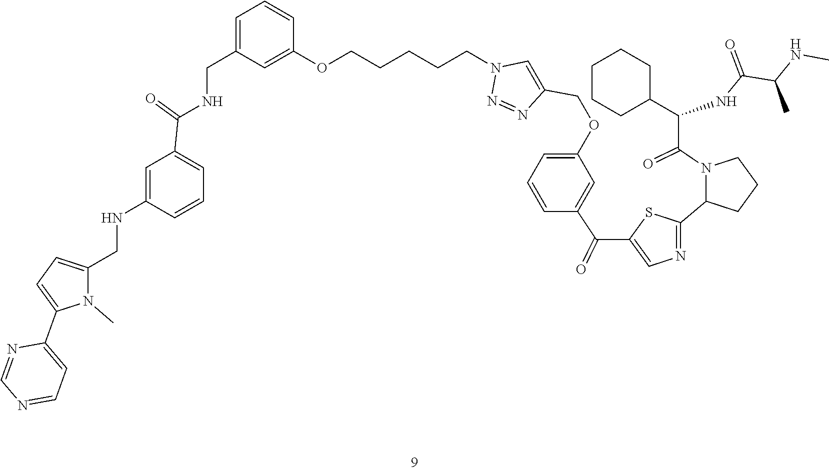 [SMILES: CN[C@@H](C)C(=O)N[C@H](C(=O)N1CCCC1c1ncc(C(=O)c2cccc(OCc3cn(CCCCCOc4cccc(CNC(=O)c5cccc(NCc6ccc(-c7ccncn7)n6C)c5)c4)nn3)c2)s1)C1CCCCC1]

### Compounds, Procedures, Analytical Data

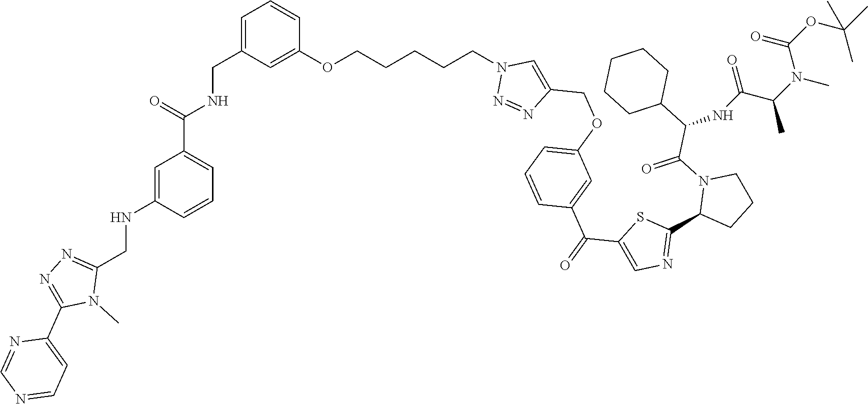 [SMILES: C[C@@H](C(=O)N[C@H](C(=O)N1CCC[C@H]1c1ncc(C(=O)c2cccc(OCc3cn(CCCCCOc4cccc(CNC(=O)c5cccc(NCc6nnc(-c7ccncn7)n6C)c5)c4)nn3)c2)s1)C1CCCCC1)N(C)C(=O)OC(C)(C)C]

To a solution of azide 7 (0.1 g, 0.18 mmol) and alkyne 2 (0.12 g, 0.19 mmol) in ethanol (1 mL), sodium ascorbate (4 mg, 0.02 mmol) in water (0.2 mL) and copper (II) acetate (4.4 mg, 0.02 mmol) in water (0.2 mL) were added successively. The mixture was stirred at ambient temperature overnight. Then the solvent was stripped off and the residue was partitioned between methylene chloride (5 mL) and water (5 mL). Organic phase was separated and dried over MgSO₄. After the solvent evaporation the product was purified by chromatography on silica, using chloroform-methanol (20 to 1, then 10 to 1) mixture as an eluent. Yield 0.22 g (99%). Colorless oil 99%. LCMS (C18 column 20×2 mm, 2.5 μm, pore size 100 Å, water-acetonitrile+0.1% TFA, gradient 5 to 87% for 3 min, retention time 1.79 min). MS (ESI) m/z 1163.9 [MH]+.

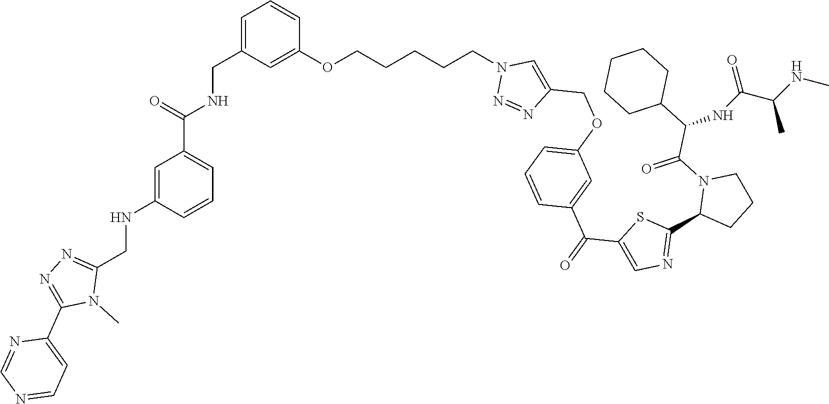 [SMILES: CN[C@@H](C)C(=O)N[C@H](C(=O)N1CCC[C@H]1c1ncc(C(=O)c2cccc(OCc3cn(CCCCCOc4cccc(CNC(=O)c5cccc(NCc6nnc(-c7ccncn7)n6C)c5)c4)nn3)c2)s1)C1CCCCC1]

D103. N-(3-((5-(4-((3-(2-((S)-1-((S)-2-cyclohexyl-2-((S)-2-(methylamino)propanamido) acetyl)pyrrolidin-2-yl)thiazole-4-carbonyl)phenoxy)methyl)-1H-1,2,3-triazol-1-yl)pentyl)oxy)benzyl)-3-(((4-methyl-5-(pyrimidin-4-yl)-4H-1,2,4-triazol-3-yl)methyl)amino)benzamide. To an ice cold solution of triazole 8 (0.21 g, 0.17 mmol) in methylene chloride (2 mL) a dioxanic 3M HCl solution (2 mL) was added. The mixture was stirred at ambient temperature overnight and then evaporated dryness. The residue was partitioned between methylene chloride (2 mL) and aqueous NaHCO₃ (10 mL). Organic phase was separated, dried over MgSO₄ and concentrated. Product was purified by chromatography, using chloroform-methanol (20 to 1, then 10 to 1) mixture as an eluent. Yield 0.13 g (64%). Colorless oil 64%. LCMS (C18 column 20×2 mm, 2.5 μm, pore size 100 Å, water-acetonitrile+0.1% TFA, gradient 5 to 87% for 10 min, retention time 5.26 min). MS (ESI) m/z 1063.3 [MH]+. ¹H NMR (400 MHz, DMSO-d₆) δ 9.32 (s, 1H), 8.96 (d, J=5.3 Hz, 1H), 8.80 (t, J=5.3 Hz, 1H), 8.48 (s, 1H), 8.24 (s, 1H), 8.17 (d, J=5.3 Hz, 1H), 7.90 (d, J=8.8 Hz, 1H), 7.73 (s, 1H), 7.69 (d, J=7.8 Hz, 1H), 7.47 (t, J=8.0 Hz, 1H), 7.33 (d, J=7.6 Hz, 1H), 7.24 (s, 1H), 7.19 (dd, J=15.7, 7.9 Hz, 2H), 7.10 (d, J=7.6 Hz, 1H), 6.90 (d, J=8.1 Hz, 1H), 6.85 (d, J=8.1 Hz, 2H), 6.76 (d, J=9.7 Hz, 1H), 6.46 (t, J=8.1 Hz, 1H), 5.38 (d, J=8.1 Hz, 1H), 5.22 (s, 2H), 4.57 (d, J=5.6 Hz, 2H), 4.49 (t, J=5.6 Hz, 1H), 4.39 (dd, J=13.7, 6.4 Hz, 4H), 4.07 (s, 3H), 3.91 (t, J=6.2 Hz, 2H), 3.78 (t, J=6.8 Hz, 2H), 2.98 (q, J=6.8 Hz, 1H), 2.37-2.23 (m, 1H), 2.22-2.14 (m, 4H), 2.10-1.96 (m, 3H), 1.93-1.81 (m, 2H), 1.77-1.66 (m, 3H), 1.65-1.50 (m, 5H), 1.44-1.30 (m, 2H), 1.16-0.90 (m, 8H).

### Example 22: Preparation of Compounds D172, D173, D174, D175, D176, D177, D117, D118, D119, D121, D122, D123, D126, D127, D128, D129, D132, D133, and D134

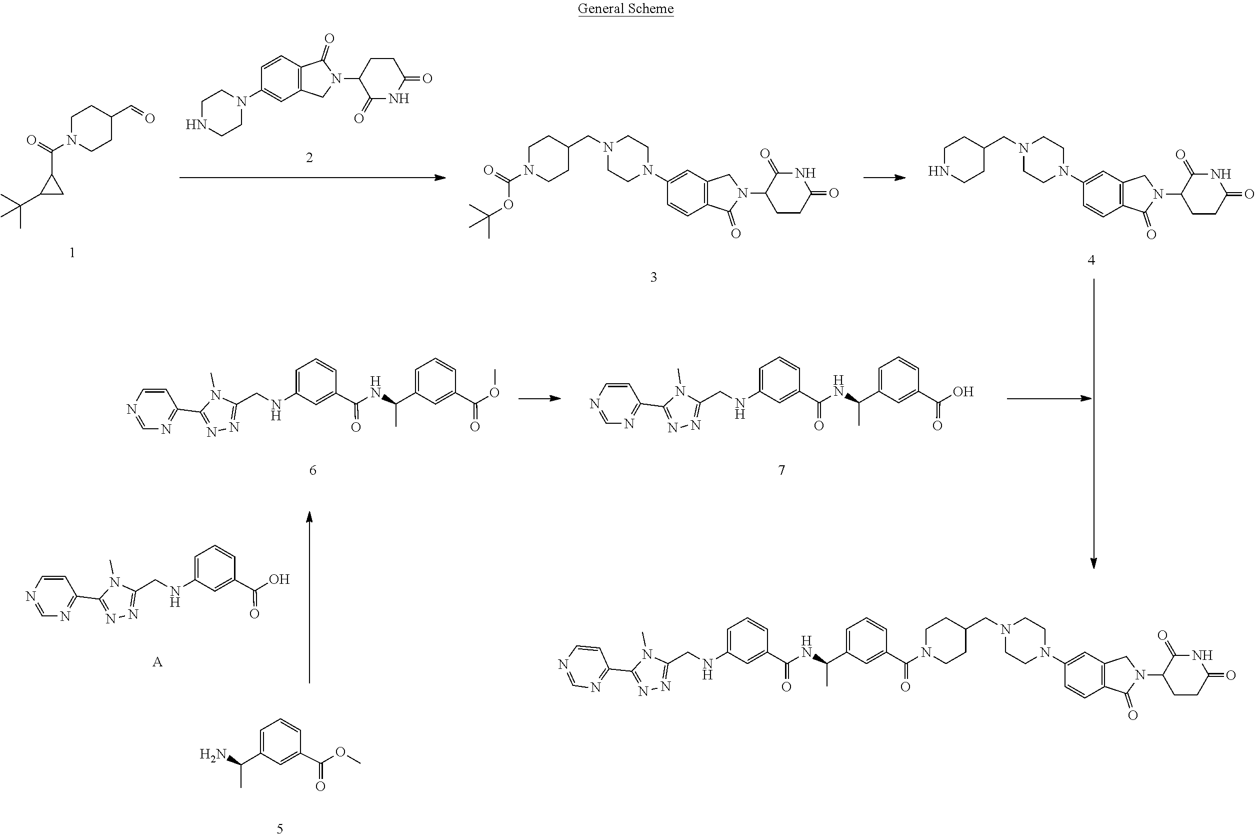

### Compounds, Procedures, and Analytical Data

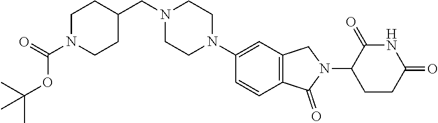 [SMILES: CC(C)(C)OC(=O)N1CCC(CN2CCN(c3ccc4c(c3)CN(C3CCC(=O)NC3=O)C4=O)CC2)CC1]

Compound 3. tert-butyl 4-((4-(2-(2,6-dioxopiperidin-3-yl)-1-oxoisoindolin-5-yl)piperazin-1-yl)methyl)piperidine-1-carboxylate. To a suspension of 3-(1-oxo-5-(piperazin-1-yl)isoindolin-2-yl)piperidine-2,6-dione hydrochloride (2) (0.340 g, 0.934 mmol) and compound 1 (0.200 g, 0.934 mmol) in 10 mL of DCM was added DIPEA (0.32 mL, 1.86 mmol) and the mixture was stirred for an hour. Then sodium triacetoxyborohydride (0.800 g, 3.77 mmol) was added. The suspension was stirred at room temperature for 12 h and quenched with saturated NaHCO₃ aqueous solution. The product was extracted with dichloromethane (3 by 20 ml). After extraction, the combined organic layer was dried. Purification by silica gel (gradient ethyl acetate/tetrahydrofuran=100:1 to 1:100 as a developing solvent) afforded compound 3 (0.401 g, 80%). LCMS (C18 column 100×4.6 mm, 5.0 μm, pore size 100 Å, water-acetonitrile+0.1% TFA, gradient 5 to 87% for 10 min, retention time 4.33 min). MS (ESI) m/z 526.4 [MH]+.

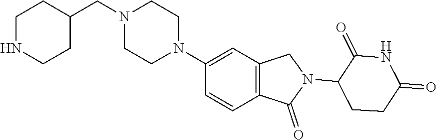 [SMILES: O=C1CCC(N2Cc3cc(N4CCN(CC5CCNCC5)CC4)ccc3C2=O)C(=O)N1]

Compound 4. 3-(1-oxo-5-(4-(piperidin-4-ylmethyl)piperazin-1-yl)isoindolin-2-yl)piperidine-2,6-dione hydrochloride. A solution of compound 3 (400 mg, 0.762 mmol, 1 eq) in dry DCM (50 mL) with 3 ml 3M HCl in dioxane (6 eq) were stirred for 24 h at r.t. The mixture was evaporated to dryness to give the crude product 4 which was used in the next step without additional purification. Yield 98%. LCMS (C18 column 100×4.6 mm, 5.0 μm, pore size 100 Å, water-acetonitrile+0.1% TFA, gradient 5 to 87% for 10 min, retention time 2.37 min). MS (ESI) m/z 426.4 [MH]+.

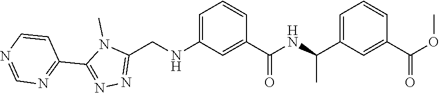 [SMILES: COC(=O)c1cccc([C@@H](C)NC(=O)c2cccc(NCc3nnc(-c4ccncn4)n3C)c2)c1]

Compound 6. (R)-methyl 3-(1-(3-((4-methyl-5-(pyrimidin-4-yl)-4H-1,2,4-triazol-3-yl)methylamino)benzamido)ethyl)benzoate. A mixture of compound 5 (1.5 g, 6.96 mmol, 1 eq), compound A (2.16 g, 6.96 mmol, 1 eq), TBTU (2.68 g, 8.35 mmol, 1.5 eq), DIPEA (3.59 g, 27.8 mmol, 4 eq) and anh. pyridine (20 mL) was stirred at ambient temperature for 12 h and concentrated under reduced pressure. The residue was diluted with CH₂Cl₂, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product (1.69 g, 52%). Colorless oil LCMS (C18 column 20×2 mm, 2.5 μm, pore size 100 Å, water-acetonitrile+0.1% TFA, gradient 5 to 87% for 3 min, retention time 1.27 min). MS (ESI) m/z 472.5 [MH]+. ¹H NMR (400 MHz, DMSO-d₆) δ 9.33 (s, 1H), 8.96 (d, J=5.3 Hz, 1H), 8.73 (d, J=7.9 Hz, 1H), 8.18 (dd, J=5.3, 1.35 Hz, 1H), 7.99 (s, 1H), 7.83 (d, J=7.8 Hz, 1H), 7.68 (d, J=7.7 Hz, 1H), 7.48 (t, J=7.7 Hz, 1H), 7.21-7.17 (m, 2H), 7.10 (d, J=7.7 Hz, 1H), 6.92 (dd, J=7.05, 1.5 Hz, 1H), 6.47 (t, J=5.5 Hz, 1H), 5.12 (m, 1H), 4.58 (d, J=5.6 Hz, 2H), 4.07 (s, 3H), 3.84 (s, 3H), 1.47 (d, J=7.07 Hz, 3H).

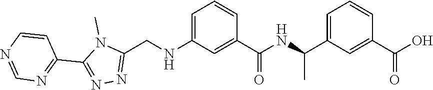 [SMILES: C[C@@H](NC(=O)c1cccc(NCc2nnc(-c3ccncn3)n2C)c1)c1cccc(C(=O)O)c1]

Compound 7. (R)-3-(1-(3-((4-methyl-5-(pyrimidin-4-yl)-4H-1,2,4-triazol-3-yl)methylamino)benzamido)ethyl)benzoic acid. To a solution of compound 6 (1.639 g, 3.48 mmol, 1 eq) in MeOH (40 mL) was added a solution KOH (0.585 g, 10.44 mmol, 3 eq) in 10 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification. Yield 89%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 458.5 [MH]+. ¹H NMR (400 MHz, DMSO-d₆) δ 12.90 (s, 1H), 9.33 (s, 1H), 8.97 (d, J=5.3 Hz, 1H), 8.71 (d, J=7.9 Hz, 1H), 8.18 (dd, J=5.3, 1.35 Hz, 1H), 7.98 (s, 1H), 7.81 (d, J=7.8 Hz, 1H), 7.64 (d, J=7.7 Hz, 1H), 7.45 (t, J=7.7 Hz, 1H), 7.19-7.11 (m, 3H), 6.90 (dd, J=7.05, 1.5 Hz, 1H), 6.47 (t, J=5.5 Hz, 1H), 5.17 (m, 1H), 4.58 (d, J=5.6 Hz, 2H), 4.07 (s, 3H), 1.47 (d, J=7.07 Hz, 3H).

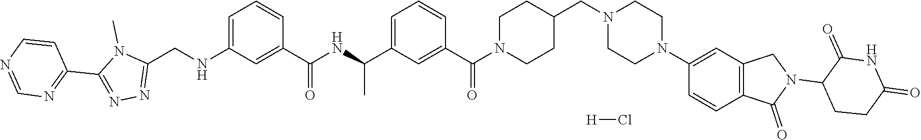 [SMILES: C[C@@H](NC(=O)c1cccc(NCc2nnc(-c3ccncn3)n2C)c1)c1cccc(C(=O)N2CCC(CN3CCN(c4ccc5c(c4)CN(C4CCC(=O)NC4=O)C5=O)CC3)CC2)c1 ; Cl]

D122. N-((1R)-1-(3-(4-((4-(2-(2,6-dioxopiperidin-3-yl)-1-oxoisoindolin-5-yl)piperazin-1-yl)methyl)piperidine-1-carbonyl)phenyl)ethyl)-3-((4-methyl-5-(pyrimidin-4-yl)-4H-1,2,4-triazol-3-yl)methylamino)benzamide hydrochloride. A mixture of compound 4 (0.1 mmol, 1 eq), compound 7 (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile) Yield 23%. LCMS (C18 column 100×4.6 mm, 5.0 μm, pore size 100 Å, water-acetonitrile+0.1% TFA, gradient 5 to 87% for 10 min, retention time 4.31 min). MS (ESI) m/z 865.8[MH]+. ¹H NMR (400 MHz, DMSO-d₆) δ 10.95 (s, 1H), 9.33 (s, 1H), 8.96 (d, J=5.4 Hz, 1H), 8.66 (d, J=8.2 Hz, 1H), 8.16 (d, J=4.1 Hz, 1H), 7.59 (d, J=8.4 Hz, 1H), 7.48-7.37 (m, 3H), 7.24-7.11 (m, 7H), 6.92 (dd, J=8.0, 1.5 Hz, 1H), 5.19-5.12 (m, 1H), 5.06 (dd, J=13.3, 5.0 Hz, 1H), 4.58 (s, 2H), 4.48 (br, 1H), 4.38-4.21 (m, 2H), 4.07 (s, 4H), 4.01 (br, 2H), 3.60 (br, 8H), 3.21-3.05 (m, 8H), 2.95-2.86 (m, 1H), 2.67-2.58 (m, 1H), 2.43-2.33 (m, 1H), 1.99-1.95 (m, 1H), 1.48 (d, J=7.0 Hz, 3H).

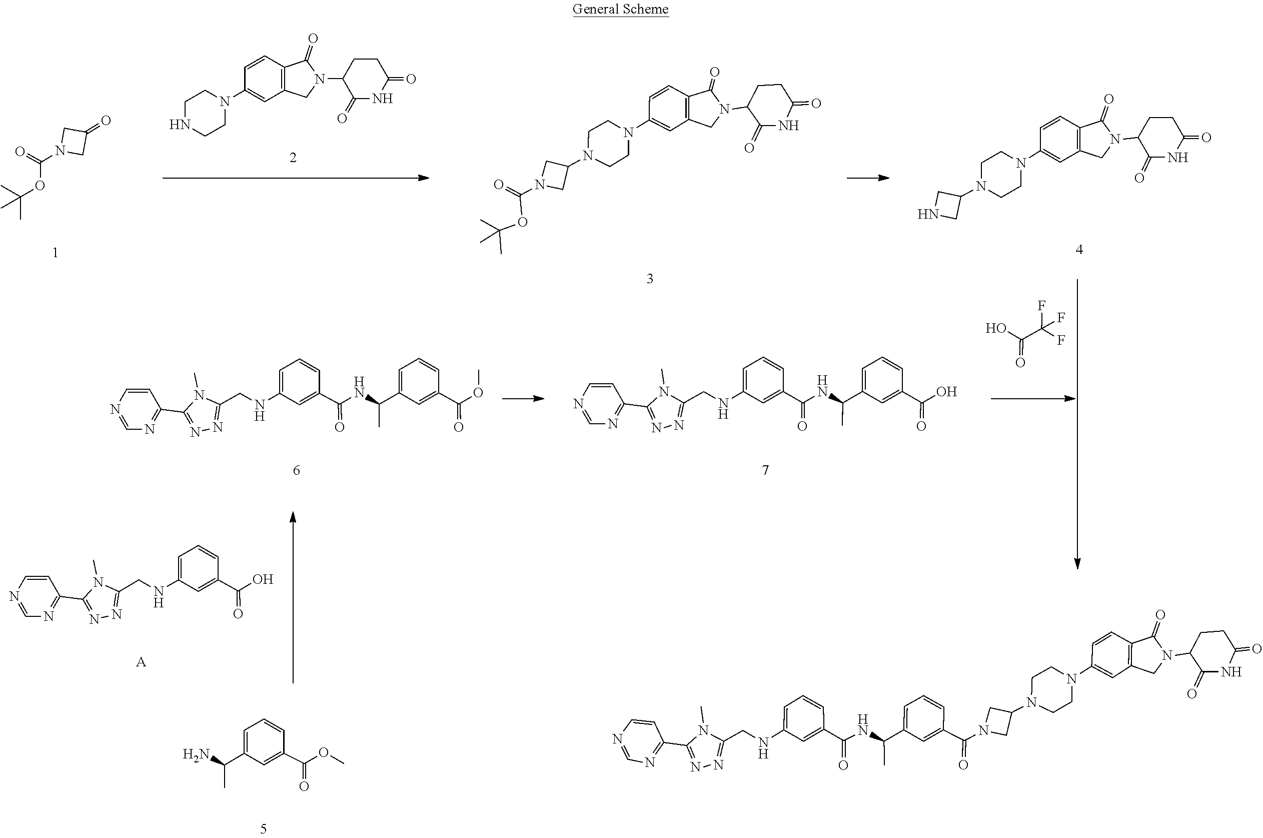

### Compounds, Procedures, and Analytical Data

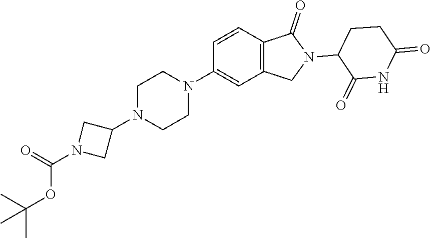 [SMILES: CC(C)(C)OC(=O)N1CC(N2CCN(c3ccc4c(c3)CN(C3CCC(=O)NC3=O)C4=O)CC2)C1]

Compound 3. tert-butyl 3-(4-(2-(2,6-dioxopiperidin-3-yl)-1-oxoisoindolin-5-yl)piperazin-1-yl)azetidine-1-carboxylate. To a suspension of 3-(1-oxo-5-(piperazin-1-yl)isoindolin-2-yl)piperidine-2,6-dione hydrochloride (2) (1.00 g, 2.74 mmol) and compound 1 (0.469 g, 2.74 mmol) in 10 mL of DCM was added DIPEA (1.0 mL, 5.49 mmol) and the mixture was stirred for an hour. Then sodium triacetoxyborohydride (2.32 g, 10.96 mmol) was added. The suspension was stirred at room temperature for 12 h and quenched with saturated NaHCO₃ aqueous solution. The product was extracted with dichloromethane (3 by 10 ml). After extraction, the combined organic layer was dried. Purification by silica gel (gradient ethyl acetate/tetrahydrofuran=100:1 to 1:100 as a developing solvent) afforded compound 3 (0.820 g, 62%). LCMS (C18 column 100×4.6 mm, 5.0 μm, pore size 100 Å, water-acetonitrile+0.1% TFA, gradient 5 to 87% for 10 min, retention time 4.52 min). MS (ESI) m/z 484.1 [MH]+

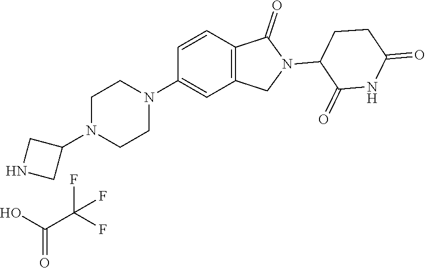

Compound 4. 3-(5-(4-(azetidin-3-yl)piperazin-1-yl)-1-oxoisoindolin-2-yl)piperidine-2,6-dione 2,2,2-trifluoroacetate/ A solution of compound 3 (1.677 mmol, 1 eq) in dry DCM (50 mL) with 1.9 ml CF₃CO₂H (6 eq) were stirred for 24 h at r.t. The mixture was evaporated to dryness to give the crude product 4 which was used in the next step without additional purification. LCMS (C18 column 100×4.6 mm, 5.0 μm, pore size 100 Å, water-acetonitrile+0.1% TFA, gradient 5 to 87% for 10 min, retention time 2.87 min). MS (ESI) m/z 384.5 [MH]+.

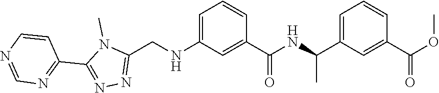 [SMILES: COC(=O)c1cccc([C@@H](C)NC(=O)c2cccc(NCc3nnc(-c4ccncn4)n3C)c2)c1]

Compound 6. (R)-methyl 3-(1-(3-((4-methyl-5-(pyrimidin-4-yl)-4H-1,2,4-triazol-3-yl)methylamino)benzamido)ethyl)benzoate. A mixture of compound 5 (1.5 g, 6.96 mmol, 1 eq), compound A (2.16 g, 6.96 mmol, 1 eq), TBTU (2.68 g, 8.35 mmol, 1.5 eq), DIPEA (3.59 g, 27.8 mmol, 4 eq) and anh. pyridine (20 mL) was stirred at ambient temperature for 12 h and concentrated under reduced pressure. The residue was diluted with CH₂C2, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product (1.69 g, 52%). Colorless oil LCMS (C18 column 20×2 mm, 2.5 μm, pore size 100 Å, water-acetonitrile+0.1% TFA, gradient 5 to 87% for 3 min, retention time 1.27 min). MS (ESI) m/z 472.5 [MH]+. ¹H NMR (400 MHz, DMSO-d₆) δ 9.33 (s, 1H), 8.96 (d, J=5.3 Hz, 1H), 8.73 (d, J=7.9 Hz, 1H), 8.18 (dd, J=5.3, 1.35 Hz, 1H), 7.99 (s, 1H), 7.83 (d, J=7.8 Hz, 1H), 7.68 (d, J=7.7 Hz, 1H), 7.48 (t, J=7.7 Hz, 1H), 7.21-7.17 (m, 2H), 7.10 (d, J=7.7 Hz, 1H), 6.92 (dd, J=7.05, 1.5 Hz, 1H), 6.47 (t, J=5.5 Hz, 1H), 5.12 (m, 1H), 4.58 (d, J=5.6 Hz, 2H), 4.07 (s, 3H), 3.84 (s, 3H), 1.47 (d, J=7.07 Hz, 3H).

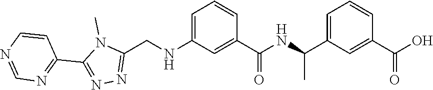 [SMILES: C[C@@H](NC(=O)c1cccc(NCc2nnc(-c3ccncn3)n2C)c1)c1cccc(C(=O)O)c1]

Compound 7. (R)-3-(1-(3-((4-methyl-5-(pyrimidin-4-yl)-4H-1,2,4-triazol-3-yl)methylamino)benzamido)ethyl)benzoic acid. To a solution of compound 6 (1.639 g, 3.48 mmol, 1 eq) in MeOH (400 mL) was added a solution KOH (0.585 g, 10.44 mmol, 3 eq) in 10 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification. Yield 89%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 458.5 [MH]+. ¹H NMR (400 MHz, DMSO-d₆) δ 12.90 (s, 1H), 9.33 (s, 1H), 8.97 (d, J=5.3 Hz, 1H), 8.71 (d, J=7.9 Hz, 1H), 8.18 (dd, J=5.3, 1.35 Hz, 1H), 7.98 (s, 1H), 7.81 (d, J=7.8 Hz, 1H), 7.64 (d, J=7.7 Hz, 1H), 7.45 (t, J=7.7 Hz, 1H), 7.19-7.11 (m, 3H), 6.90 (dd, J=7.05, 1.5 Hz, 1H), 6.47 (t, J=5.5 Hz, 1H), 5.17 (m, 1H), 4.58 (d, J=5.6 Hz, 2H), 4.07 (s, 3H), 1.47 (d, J=7.07 Hz, 3H).

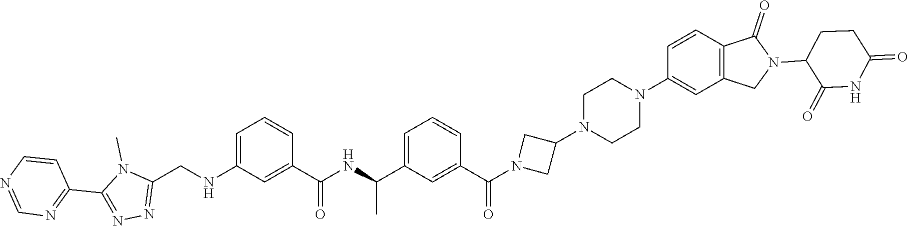 [SMILES: C[C@@H](NC(=O)c1cccc(NCc2nnc(-c3ccncn3)n2C)c1)c1cccc(C(=O)N2CC(N3CCN(c4ccc5c(c4)CN(C4CCC(=O)NC4=O)C5=O)CC3)C2)c1]

D172. N-((1R)-1-(3-(3-(4-(2-(2,6-dioxopiperidin-3-yl)-1-oxoisoindolin-5-yl)piperazin-1-yl)azetidine-1-carbonyl)phenyl)ethyl)-3-((4-methyl-5-(pyrimidin-4-yl)-4H-1,2,4-triazol-3-yl)methylamino)benzamide hydrochloride. A mixture of compound 4 (0.1 mmol, 1 eq), compound 7 (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile) Yield 57%. LCMS (C18 column 100×4.6 mm, 5.0 μm, pore size 100 Å, water-acetonitrile+0.1% TFA, gradient 5 to 87% for 10 min, retention time 4.17 min). MS (ESI) m/z 823.4[MH]+. ¹H NMR (400 MHz, DMSO-d₆) δ 10.94 (s, 1H), 9.32 (s, 1H), 8.97 (d, J=5.4 Hz, 1H), 8.73 (d, J=8.2 Hz, 1H), 8.15 (d, J=4.1 Hz, 1H), 7.67 (s, 1H), 7.58-7.56 (m, 2H), 7.51-7.42 (m, 2H), 7.22-7.13 (m, 5H), 6.91 (dd, J=8.0, 1.5 Hz, 1H), 5.21 (m, 1H), 5.08 (dd, J=13.3, 5.0 Hz, 1H), 4.57 (s, 3H), 4.43 (br, 1H), 4.36-4.21 (m, 5H), 4.06 (s, 4H), 3.12 (br, 4H), 2.94-2.87 (m, 1H), 2.68-2.57 (m, 1H), 2.43-2.33 (m, 1H), 1.99-1.94 (m, 1H), 1.50 (d, J=7.0 Hz, 3H).

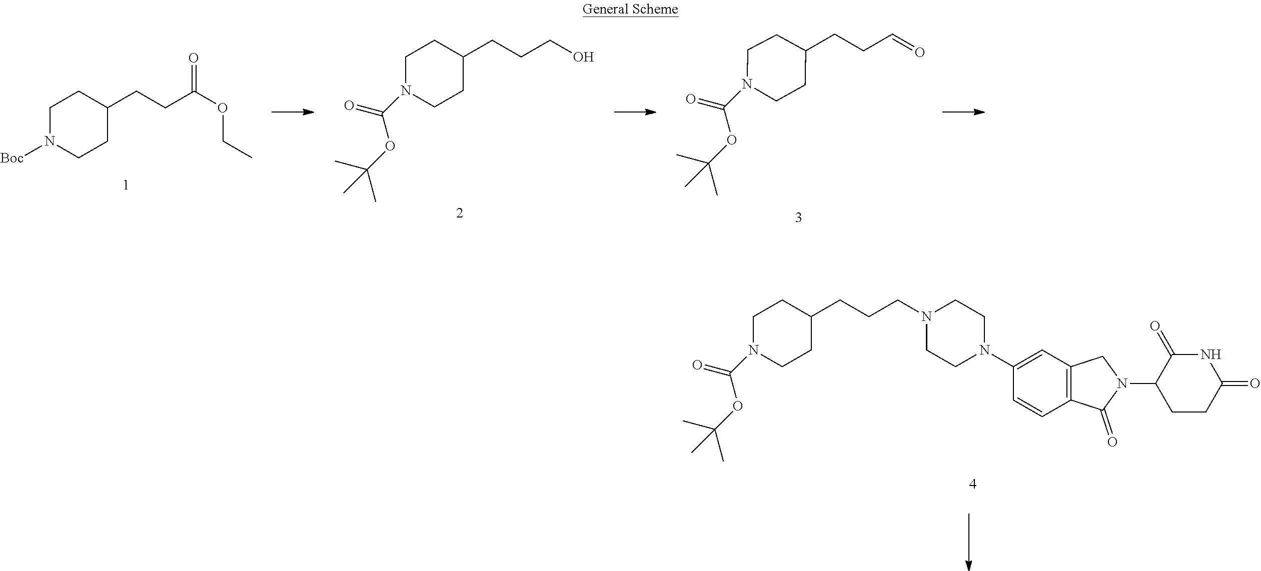 [SMILES: CCOC(=O)CCC1CCN(C(=O)OC(C)(C)C)CC1 ; CC(C)(C)OC(=O)N1CCC(CCCO)CC1 ; CC(C)(C)OC(=O)N1CCC(CCC=O)CC1 ; CC(C)(C)OC(=O)N1CCC(CCCN2CCN(c3ccc4c(c3)CN(C3CCC(=O)NC3=O)C4=O)CC2)CC1]

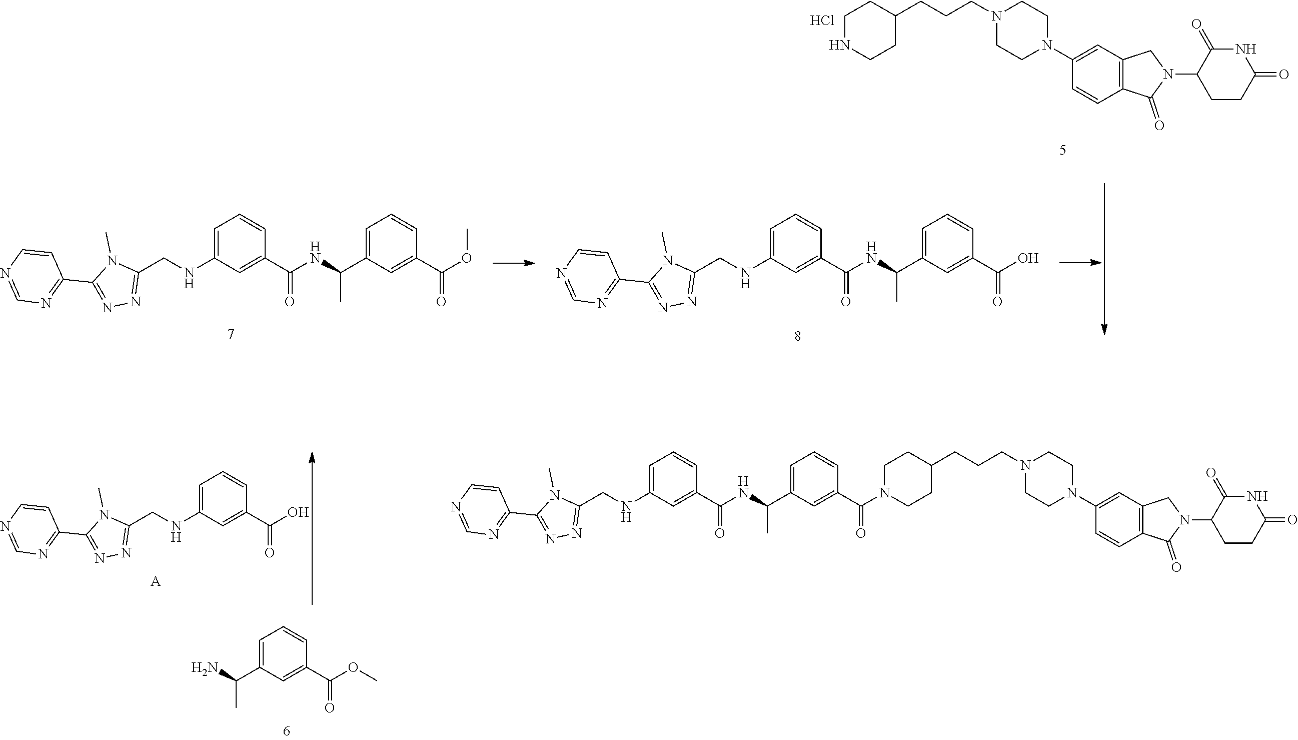

### Compounds, Procedures, and Analytical Data

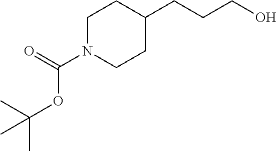 [SMILES: CC(C)(C)OC(=O)N1CCC(CCCO)CC1]

Compound 2. tert-butyl 4-(3-hydroxypropyl)piperidine-1-carboxylate. To a mixture of compound 1 (250 mg, 0.93 mmol) in Et2O (5 mL), LiBH₄ (30 mg, 1.39 mmol) was added, followed by methanol (45 mg, 1.39 mmol) at 0° C. The reaction mixture was stirred at ambient temperature overnight. The solvent was evaporated and the residue was partitioned between DCM (5 ml) and water, contains with citric acid (0.7 g, 3.64 mmol in water 10 ml). The extract was separated and dried over Na₂SO₄. The crude product 2 was used further without additional purification. LCMS (C18 column 20×2 mm, 2.5 m, pore size 100 Å, water-acetonitrile+0.1% TFA, gradient 5 to 87% for 3 min, retention time 1.41 min). MS (ESI) m/z 244.4 [MH]+. ¹H NMR (400 MHz, CDCl₃) δ 4.36 (t, J=5.1 Hz, 1H), 3.92 (br, 2H, 3.39-3.36 (m, 2H), 2.66 (br, 2H), 1.62 (d, J=12.7 Hz, 2H), 1.43-1.33 (m, 12H), 1.22-1.17 (m, 2H), 0.97-0.88 (m, 2H).

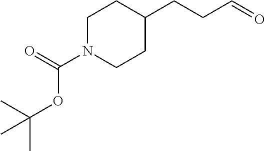 [SMILES: CC(C)(C)OC(=O)N1CCC(CCC=O)CC1]

Compound 3. Tert-butyl 4-(3-oxopropyl)piperidine-1-carboxylate. A flask was charged with 10 mL of dichloromethane and oxalyl chloride (1.14 mmol). The solution was stirred and cooled to −60° C. then dimethyl sulfoxide (1.69 mmol) in 5 mL of dichloromethane was added dropwise at a rapid rate. After 5 min compound 10 (0.22 mmol) in 5 ml of dichloromethane was added dropwise maintaining the temperature at −50 to −60° C. After another 15 min, triethylamine (0.5 ml, 3.59 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 0.115 g (95%) of crude product 3 that used further without additional purification.

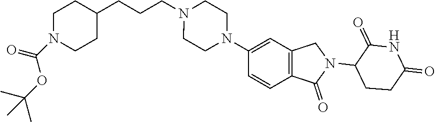 [SMILES: CC(C)(C)OC(=O)N1CCC(CCCN2CCN(c3ccc4c(c3)CN(C3CCC(=O)NC3=O)C4=O)CC2)CC1]

Compound 4. tert-butyl 4-(3-(4-(2-(2,6-dioxopiperidin-3-yl)-1-oxoisoindolin-5-yl)piperazin-1-yl)propyl)piperidine-1-carboxylate. To a suspension of 3-(1-oxo-5-(piperazin-1-yl)isoindolin-2-yl)piperidine-2,6-dione hydrochloride (0.317 g, 0.87 mmol) and compound 3 (0.210 g, 0.87 mmol) in 10 mL of DCM was added DIPEA (0.300 mL, 1.74 mmol) and the mixture was stirred for an hour. Then sodium triacetoxyborohydride (0.737 g, 3.48 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄.

Purification by silica gel (gradient ethyl acetate/tetrahydrofuran=100:1 to 1:100 as a developing solvent) afforded compound 4 (0.269 g, 56%). LCMS (C18 column 100×4.6 mm, 5.0 μm, pore size 100 Å, water-acetonitrile+0.1% TFA, gradient 5 to 87% for 10 min, retention time 4.69 min). MS (ESI) m/z 554.5 [MH]+

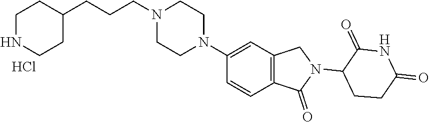 [SMILES: O=C1CCC(N2Cc3cc(N4CCN(CCCC5CCNCC5)CC4)ccc3C2=O)C(=O)N1 ; Cl]

Compound 5. 3-(1-oxo-5-(4-(3-(piperidin-4-yl)propyl)piperazin-1-yl)isoindolin-2-yl)piperidine-2,6-dione hydrochloride. A solution of compound 4 (0.486 mmol, 1 eq) in dry DCM (50 mL) with 1.9 ml 3M HCl in dioxane (6 eq) were stirred for 24 h at r.t. The mixture was evaporated to dryness to give the crude product 5 which was used in the next step without additional purification. Yield 98%. LCMS (C18 column 100×4.6 mm, 5.0 μm, pore size 100 Å, water-acetonitrile+0.1% TFA, gradient 5 to 87% for 10 min, retention time 3.00 min). MS (ESI) m/z 454.7 [MH]+. ¹H NMR (400 MHz, CDCl₃) δ 11.39 (br, 1H), 10.95 (s, 1H), 9.05 (br, 1H), 8.86 (br, 1H), 7.57 (d, J=8.5 Hz, 1H), 7.17-1.72 (m, 2H), 5.08 (dd, J=13.3, 5.2 Hz, 1H), 4.38-4.21 (m, 2H), 4.00 (d, J=14.0 Hz, 2H), 3.54-3.50 (m, 2H), 3.35 (t, J=12.6 Hz, 2H), 3.24 (d, J=13.1 Hz, 2H), 3.12-3.03 (m, 4H), 2.95-2.75 (m, 3H), 2.62-2.54 (m, 1H), 2.43-2.33 (m, 1H), 2.00-1.94 (m, 1H), 1.82-1.75 (m, 4H), 1.56-1.50 (m, 1H), 1.37-1.19 (m, 4H).

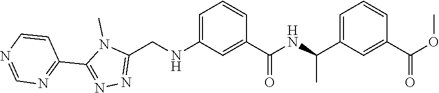 [SMILES: COC(=O)c1cccc([C@@H](C)NC(=O)c2cccc(NCc3nnc(-c4ccncn4)n3C)c2)c1]

Compound 7. (R)-methyl 3-(1-(3-((4-methyl-5-(pyrimidin-4-yl)-4H-1,2,4-triazol-3-yl)methylamino)benzamido)ethyl)benzoate. A mixture of compound 6 (1.5 g, 6.96 mmol, 1 eq), compound A (2.16 g, 6.96 mmol, 1 eq), TBTU (2.68 g, 8.35 mmol, 1.5 eq), DIPEA (3.59 g, 27.8 mmol, 4 eq), and anh. pyridine (20 mL) was stirred at ambient temperature for 12 h and concentrated under reduced pressure. The residue was diluted with CH₂Cl₂, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product (1.69 g, 52%). Colorless oil LCMS (C18 column 20×2 mm, 2.5 μm, pore size 100 Å, water-acetonitrile+0.1% TFA, gradient 5 to 87% for 3 min, retention time 1.27 min). MS (ESI) m/z 472.5 [MH]+

¹H NMR (400 MHz, DMSO-d₆) δ 9.33 (s, 1H), 8.96 (d, J=5.3 Hz, 1H), 8.73 (d, J=7.9 Hz, 1H), 8.18 (dd, J=5.3, 1.35 Hz, 1H), 7.99 (s, 1H), 7.83 (d, J=7.8 Hz, 1H), 7.68 (d, J=7.7 Hz, 1H), 7.48 (t, J=7.7 Hz, 1H), 7.21-7.17 (m, 2H), 7.10 (d, J=7.7 Hz, 1H), 6.92 (dd, J=7.05, 1.5 Hz, 1H), 6.47 (t, J=5.5 Hz, 1H), 5.12 (m, 1H), 4.58 (d, J=5.6 Hz, 2H), 4.07 (s, 3H), 3.84 (s, 3H), 1.47 (d, J=7.07 Hz, 3H).

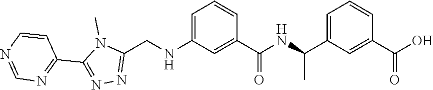 [SMILES: C[C@@H](NC(=O)c1cccc(NCc2nnc(-c3ccncn3)n2C)c1)c1cccc(C(=O)O)c1]

Compound 8. (R)-3-(1-(3-((4-methyl-5-(pyrimidin-4-yl)-4H-1,2,4-triazol-3-yl)methylamino)benzamido)ethyl)benzoic acid. To a solution of compound 7 (1.639 g, 3.48 mmol, 1 eq) in MeOH (400 mL) was added a solution KOH (0.585 g, 10.44 mmol, 3 eq) in 10 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification. Yield 83%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 458.5 [MH]+. ¹H NMR (400 MHz, DMSO-d₆) δ 12.90 (s, 1H), 9.33 (s, 1H), 8.97 (d, J=5.3 Hz, 1H), 8.71 (d, J=7.9 Hz, 1H), 8.18 (dd, J=5.3, 1.35 Hz, 1H), 7.98 (s, 1H), 7.81 (d, J=7.8 Hz, 1H), 7.64 (d, J=7.7 Hz, 1H), 7.45 (t, J=7.7 Hz, 1H), 7.19-7.11 (m, 3H), 6.90 (dd, J=7.05, 1.5 Hz, 1H), 6.47 (t, J=5.5 Hz, 1H), 5.17 (m, 1H), 4.58 (d, J=5.6 Hz, 2H), 4.07 (s, 3H), 1.47 (d, J=7.07 Hz, 3H).

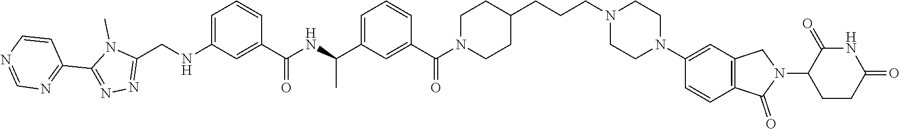 [SMILES: C[C@@H](NC(=O)c1cccc(NCc2nnc(-c3ccncn3)n2C)c1)c1cccc(C(=O)N2CCC(CCCN3CCN(c4ccc5c(c4)CN(C4CCC(=O)NC4=O)C5=O)CC3)CC2)c1]

D119. N-((1R)-1-(3-(4-(3-(4-(2-(2,6-dioxopiperidin-3-yl)-1-oxoisoindolin-5-yl)piperazin-1-yl)propyl)piperidine-1-carbonyl)phenyl)ethyl)-3-((4-methyl-5-(pyrimidin-4-yl)-4H-1,2,4-triazol-3-yl)methylamino)benzamide hydrochloride. A mixture of compound 5 (0.1 mmol, 1 eq), compound 8 (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×200 mml, S-10 μm, 12 nm, gradient water-acetonitrile) Yield 57%. LCMS (C18 column 100×4.6 mm, 5.0 μm, pore size 100 Å, water-acetonitrile+0.1% TFA, gradient 5 to 87% for 10 min, retention time 4.65 min). MS (ESI) m/z 893.9[MH]+. ¹H NMR (400 MHz, CDCl₃) δ 10.95 (s, 1H), 9.52 (br, 1H), 9.33 (s, 1H), 8.98 (d, J=5.3 Hz, 1H), 8.67 (d, J=8.1 Hz, 1H), 8.16 (dd, J=5.3, 1.4 Hz, 1H), 7.60 (d, J=4.4 Hz, 1H), 7.47-7.36 (m, 3H), 7.23-7.11 (m, 7H), 6.90 (dd, J=7.5, 2.4 Hz, 1H), 5.16 (t, J=8.4 Hz, 1H), 5.08 (dd, J=13.3, 5.0 Hz, 1H), 4.57 (s, 2H), 4.47 (br, 1H), 4.37-4.20 (m, 2H), 4.07-4.01 (m, 6H), 3.21 (br, 3H), 3.17-3.07 (br, 7H), 2.96-2.86 (m, 1H), 2.68-2.57 (m, 1H), 2.41-2.33 (m, 1H), 2.00-1.94 (m, 1H), 1.79-1.63 (m, 3H), 1.54-1.46 (m, 5H), 1.26-1.20 (m, 2H), 1.11-1.02 (m, 2H).

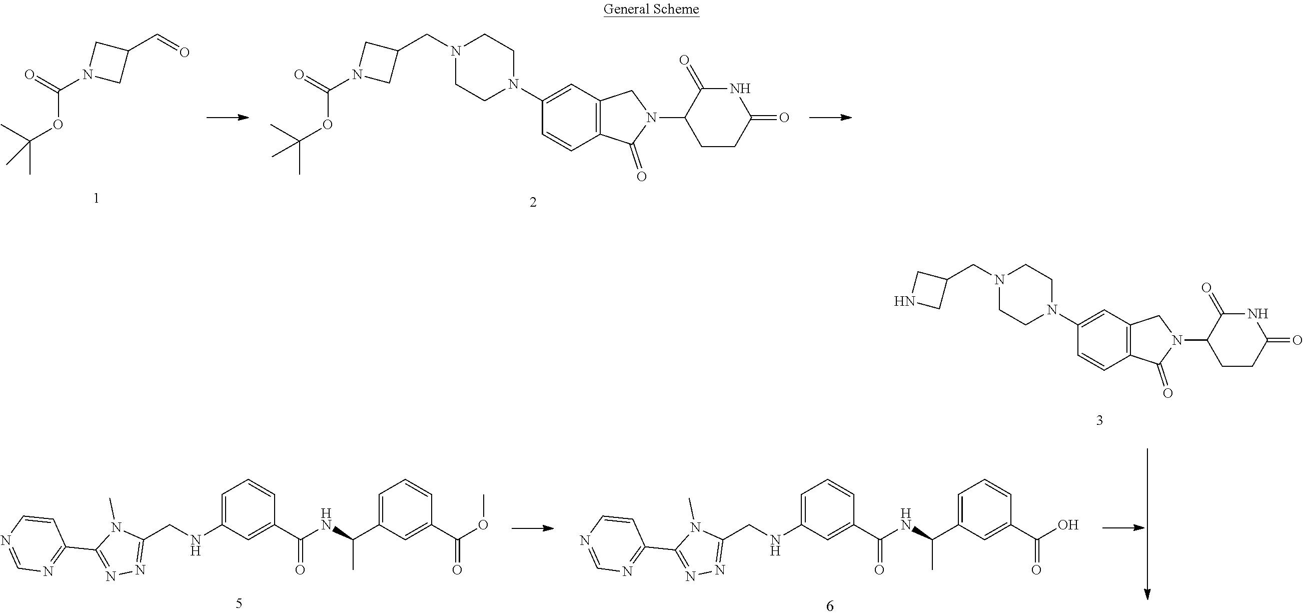 [SMILES: CC(C)(C)OC(=O)N1CC(C=O)C1 ; CC(C)(C)OC(=O)N1CC(CN2CCN(c3ccc4c(c3)CN(C3CCC(=O)NC3=O)C4=O)CC2)C1 ; O=C1CCC(N2Cc3cc(N4CCN(CC5CNC5)CC4)ccc3C2=O)C(=O)N1 ; COC(=O)c1cccc([C@@H](C)NC(=O)c2cccc(NCc3nnc(-c4ccncn4)n3C)c2)c1 ; C[C@@H](NC(=O)c1cccc(NCc2nnc(-c3ccncn3)n2C)c1)c1cccc(C(=O)O)c1]

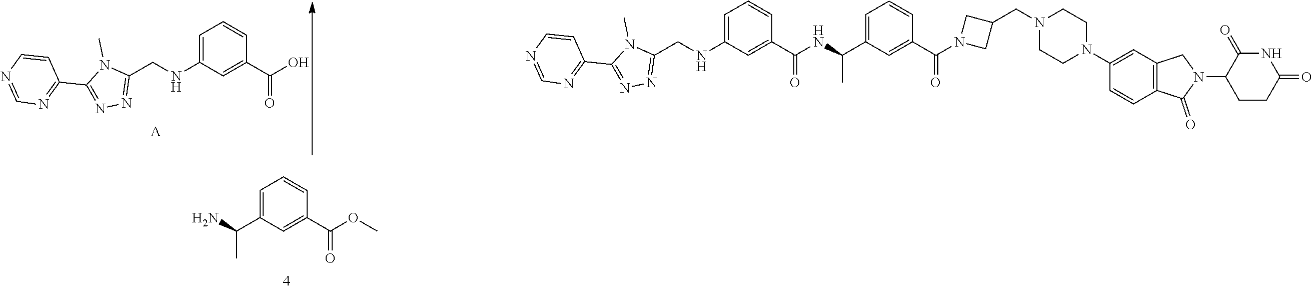

### Compounds, Procedures, and Analytical Data

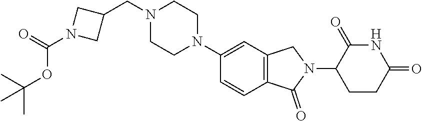 [SMILES: CC(C)(C)OC(=O)N1CC(CN2CCN(c3ccc4c(c3)CN(C3CCC(=O)NC3=O)C4=O)CC2)C1]

Compound 2. tert-butyl 3-((4-(2-(2,6-dioxopiperidin-3-yl)-1-oxoisoindolin-5-yl)piperazin-1-yl)methyl)azetidine-1-carboxylate. To a suspension of 3-(1-oxo-5-(piperazin-1-yl)isoindolin-2-yl)piperidine-2,6-dione hydrochloride (0.393 g, 1.08 mmol) and compound 1 (0.210 g, 0.1.08 mmol) in 10 mL of DCM was added DIPEA (0.380 mL, 2.16 mmol) and the mixture was stirred for an hour. Then sodium triacetoxyborohydride (0.915 g, 4.32 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 2 (0.402 g, 74%). LCMS (C18 column 100×4.6 mm, 5.0 μm, pore size 100 Å, water-acetonitrile+0.1% TFA, gradient 5 to 87% for 10 min, retention time 4.13 min). MS (ESI) m/z 498.4 [MH]+

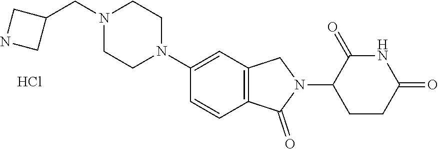 [SMILES: O=C1CCC(N2Cc3cc(N4CCN(CC5CNC5)CC4)ccc3C2=O)C(=O)N1 ; Cl]

Compound 3. 3-(5-(4-(azetidin-3-ylmethyl)piperazin-1-yl)-1-oxoisoindolin-2-yl)piperidine-2,6-dione hydrochloride. A solution of compound 2 (0.805 mmol, 1 eq) in dry DCM (50 mL) with 1.2 ml 3M HCl in dioxane in dioxane (5 eq) were stirred for 24 h at r.t. The mixture was evaporated to dryness to give the crude product 3 which was used in the next step without additional purification. Colorless oil LCMS (C18 column 20×2 mm, 2.5 μm, pore size 100 Å, water-acetonitrile+0.1% TFA, gradient 5 to 87% for 3 min, retention time 1.74 min). MS (ESI) m/z 398.5 [MH]+.

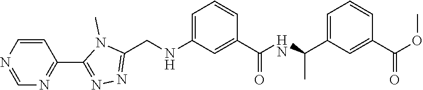 [SMILES: COC(=O)c1cccc([C@@H](C)NC(=O)c2cccc(NCc3nnc(-c4ccncn4)n3C)c2)c1]

Compound 5. (R)-methyl 3-(1-(3-((4-methyl-5-(pyrimidin-4-yl)-4H-1,2,4-triazol-3-yl)methylamino)benzamido)ethyl)benzoate. A mixture of compound 4 (1.5 g, 6.96 mmol, 1 eq), compound A (2.16 g, 6.96 mmol, 1 eq), TBTU (2.68 g, 8.35 mmol, 1.5 eq), DIPEA (3.59 g, 27.8 mmol, 4 eq), and anh. pyridine (20 mL) was stirred at ambient temperature for 12 h and concentrated under reduced pressure. The residue was diluted with CH₂C2, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product (1.69 g, 52%). Colorless oil LCMS (C18 column 20×2 mm, 2.5 μm, pore size 100 Å, water-acetonitrile+0.1% TFA, gradient 5 to 87% for 3 min, retention time 1.27 min). MS (ESI) m/z 472.5 [MH]+. ¹H NMR (400 MHz, DMSO-d₆) δ 9.33 (s, 1H), 8.96 (d, J=5.3 Hz, 1H), 8.73 (d, J=7.9 Hz, 1H), 8.18 (dd, J=5.3, 1.35 Hz, 1H), 7.99 (s, 1H), 7.83 (d, J=7.8 Hz, 1H), 7.68 (d, J=7.7 Hz, 1H), 7.48 (t, J=7.7 Hz, 1H), 7.21-7.17 (m, 2H), 7.10 (d, J=7.7 Hz, 1H), 6.92 (dd, J=7.05, 1.5 Hz, 1H), 6.47 (t, J=5.5 Hz, 1H), 5.12 (m, 1H), 4.58 (d, J=5.6 Hz, 2H), 4.07 (s, 3H), 3.84 (s, 3H), 1.47 (d, J=7.07 Hz, 3H).

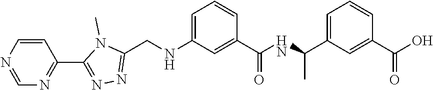 [SMILES: C[C@@H](NC(=O)c1cccc(NCc2nnc(-c3ccncn3)n2C)c1)c1cccc(C(=O)O)c1]

Compound 6. (R)-3-(1-(3-((4-methyl-5-(pyrimidin-4-yl)-4H-1,2,4-triazol-3-yl)methylamino)benzamido)ethyl)benzoic acid. To a solution of compound 5 (1.639 g, 3.48 mmol, 1 eq) in MeOH (400 mL) was added a solution KOH (0.585 g, 10.44 mmol, 3 eq) in 10 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification. LCMS (C18 column 20×2 mm, 2.5 μm, pore size 100 Å, water-acetonitrile+0.1% TFA, gradient 5 to 87% for 3 min, retention time 1.15 min). MS (ESI) m/z 458.5 [MH]+. ¹H NMR (400 MHz, DMSO-d₆) δ 12.90 (s, 1H), 9.33 (s, 1H), 8.97 (d, J=5.3 Hz, 1H), 8.71 (d, J=7.9 Hz, 1H), 8.18 (dd, J=5.3, 1.35 Hz, 1H), 7.98 (s, 1H), 7.81 (d, J=7.8 Hz, 1H), 7.64 (d, J=7.7 Hz, 1H), 7.45 (t, J=7.7 Hz, 1H), 7.19-7.11 (m, 3H), 6.90 (dd, J=7.05, 1.5 Hz, 1H), 6.47 (t, J=5.5 Hz, 1H), 5.17 (m, 1H), 4.58 (d, J=5.6 Hz, 2H), 4.07 (s, 3H), 1.47 (d, J=7.07 Hz, 3H).

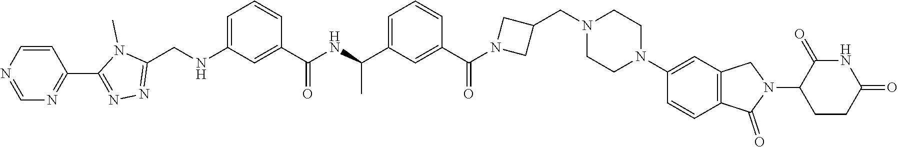 [SMILES: C[C@@H](NC(=O)c1cccc(NCc2nnc(-c3ccncn3)n2C)c1)c1cccc(C(=O)N2CC(CN3CCN(c4ccc5c(c4)CN(C4CCC(=O)NC4=O)C5=O)CC3)C2)c1]

D121. N-((1R)-1-(3-(3-((4-(2-(2,6-dioxopiperidin-3-yl)-1-oxoisoindolin-5-yl)piperazin-1-yl)methyl)azetidine-1-carbonyl)phenyl)ethyl)-3-((4-methyl-5-(pyrimidin-4-yl)-4H-1,2,4-triazol-3-yl)methylamino)benzamide hydrochloride. A mixture of compound 3 (0.1 mmol, 1 eq), compound 6 (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile) Yield 39%. LCMS (C18 column 100×4.6 mm, 5.0 μm, pore size 100 Å, water-acetonitrile+0.1% TFA, gradient 5 to 87% for 10 min, retention time 4.44 min). MS (ESI) m/z 837.9 [MH]+. ¹H NMR (400 MHz, CDCl₃) δ 10.95 (s, 1H), 9.71 (br, 1H), 9.33 (s, 1H), 8.98 (d, J=5.3 Hz, 1H), 8.70 (d, J=8.1 Hz, 1H), 8.16 (dd, J=5.3, 1.4 Hz, 1H), 7.65 (br, 1H), 7.60-7.53 (m, 2H), 7.48-7.40 (m, 2H), 7.23-7.11 (m, 6H), 6.90 (dd, J=7.5, 2.4 Hz, 1H), 5.16 (m, 1H), 5.08 (dd, J=13.3, 5.0 Hz, 1H), 4.57 (s, 2H), 4.53-4.46 (m, 1H), 4.37-4.20 (m, 2H), 4.14-4.10 (m, 1H), 4.07 (s, 2H), 3.50 (br, 6H), 3.17-3.07 (br, 7H), 2.95-2.86 (m, 1H), 2.69-2.58 (m, 1H), 2.43-2.32 (m, 1H), 2.00-1.94 (m, 1H), 1.48 (d, 3H).

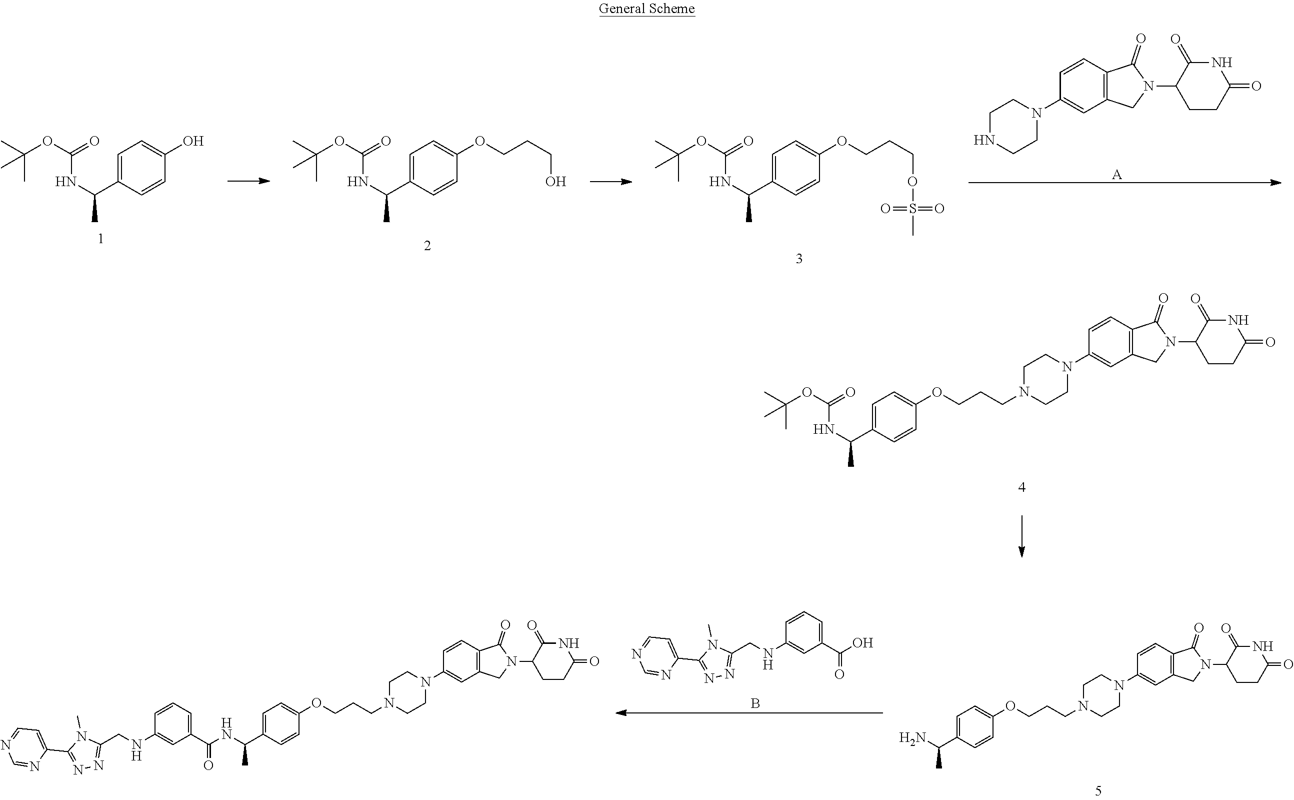

### Compounds, Procedures, and Analytical Data

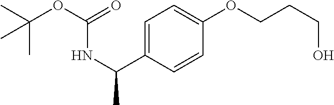 [SMILES: C[C@@H](NC(=O)OC(C)(C)C)c1ccc(OCCCO)cc1]

Compound 2. (R)-tert-butyl 1-(4-(3-hydroxypropoxy)phenyl)ethylcarbamate. A mixture of compound 1 (2.5 mmol, 1 eq), bromopropanol (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 2. ¹H NMR (400 MHz, DMSO-d₆) δ 7.24 (d, J=6.2 Hz, 1H), 7.19 (d, J=7.4 Hz, 2H), 6.85 (d, J=7.2 Hz, 2H), 7.36-7.27 (m, 5H), 4.52 (t, J=6.2 Hz, 1H), 4.01-3.98 (m, 2H), 3.56-3.52 (m, 2H), 3.46-3.39 (m, 1H), 1.86-1.82 (m, 2H), 1.35 (s, 9H), 1.25 (d, J=6.7 Hz, 3H).

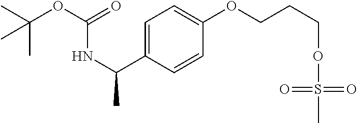 [SMILES: C[C@@H](NC(=O)OC(C)(C)C)c1ccc(OCCCOS(C)(=O)=O)cc1]

Compound 3. (R)-3-(4-(1-(tert-butoxycarbonylamino)ethyl)phenoxy)propyl methanesulfonate. A solution of compound 2 (161 mg, 0.546 mmol) in CH₂Cl₂ was mixed with 0.1 mL of DIPEA (0.819 mmol) and cooled to 0° C. 75 mg of MsCl (0.655 mmol) was added dropwise. The mixture was stirred at room temperature for 2 hours, washed with water, dried over Na₂SO₄ and evaporated. The product was used without purification. Yield 203 mg (98%)

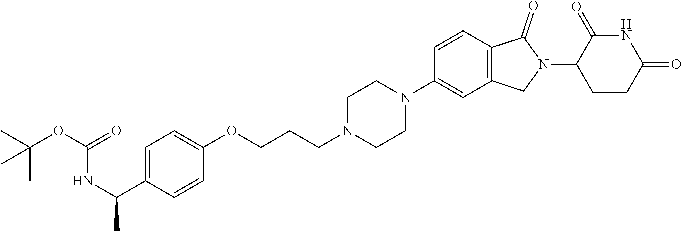 [SMILES: C[C@@H](NC(=O)OC(C)(C)C)c1ccc(OCCCN2CCN(c3ccc4c(c3)CN(C3CCC(=O)NC3=O)C4=O)CC2)cc1]

Compound 4. tert-butyl (R)-1-(4-(3-(4-(2-(2,6-dioxopiperidin-3-yl)-1-oxoisoindolin-5-yl)piperazin-1-yl)propoxy)phenyl)ethylcarbamate. A solution of A (239 mg, 0.656 mmol), compound 3 (204 mg, 0.547 mmol), DIPEA (211 mg, 1.64 mmol), KI (181 mg, 1.09 mmol) and tetrabuthyl ammonium iodide (18 mg, 0.05 mmol) in 10 mL of DMSO was stirred for an 48 hour at 50° C. The product was extracted with dichloromethane (3 by 10 ml). After extraction, the combined organic layer was dried over Na₂SO₄. Purification by silica gel column chromatography (gradient ethyl acetate/tetrahydrofuran=100:1 to 1:100 as a developing solvent) afforded compound 4 (53 mg, 16%). LCMS (C18 column 100×4.6 mm, 5.0 μm, pore size 100 Å, water-acetonitrile+0.1% TFA, gradient 5 to 87% for 10 min, retention time 4.84 min). MS (ESI) m/z 606.4 [MH]+

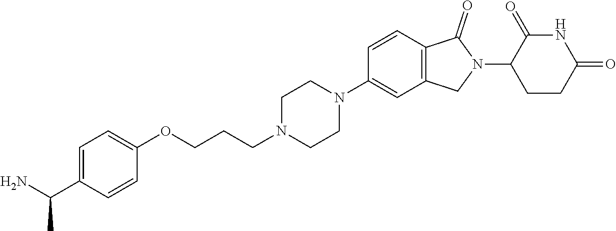 [SMILES: C[C@@H](N)c1ccc(OCCCN2CCN(c3ccc4c(c3)CN(C3CCC(=O)NC3=O)C4=O)CC2)cc1]

Compound 5. 3-(5-(4-(3-(4-((R)-1-aminoethyl)phenoxy)propyl)piperazin-1-yl)-1-oxoisoindolin-2-yl)piperidine-2,6-dione. A solution of compound 4 (0.805 mmol, 1 eq) in dry DCM (50 mL) with 1.2 ml 3M HCl in dioxane (5 eq) were stirred for 24 h at r.t. The mixture was evaporated to dryness to give the crude product 4 which was used in the next step without additional purification. Yield 97%. LCMS (C18 column 100×4.6 mm, 5.0 μm, pore size 100 Å, water-acetonitrile+0.1% TFA, gradient 5 to 87% for 10 min, retention time 3.39 min). MS (ESI) m/z 506.6 [MH]+.

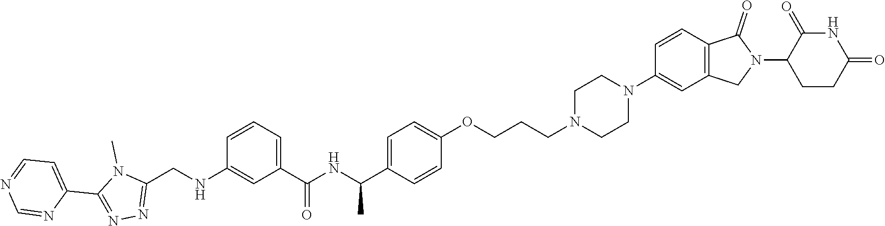 [SMILES: C[C@@H](NC(=O)c1cccc(NCc2nnc(-c3ccncn3)n2C)c1)c1ccc(OCCCN2CCN(c3ccc4c(c3)CN(C3CCC(=O)NC3=O)C4=O)CC2)cc1]

D173. N-((1R)-1-(4-(3-(4-(2-(2,6-dioxopiperidin-3-yl)-1-oxoisoindolin-5-yl)piperazin-1-yl)propoxy)phenyl)ethyl)-3-((4-methyl-5-(pyrimidin-4-yl)-4H-1,2,4-triazol-3-yl)methylamino)benzamide hydrochloride. A mixture of compound 5 (0.1 mmol, 1 eq), compound B (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile) Yield 56%. LCMS (C18 column 100×4.6 mm, 5.0 μm, pore size 100 Å, water-acetonitrile+0.1% TFA, gradient 5 to 87% for 10 min, retention time 4.40 min). MS (ESI) m/z 798.1 [MH]+

¹H NMR (400 MHz, DMSO-d₆) δ 10.95 (s, 1H), 9.59 (br, 1H), 9.33 (s, 1H), 8.98 (d, J=5.3 Hz, 1H), 8.55 (d, J=8.2 Hz, 1H), 8.16 (dd, J=6.6, 1.4 Hz, 1H), 7.60 (d, J=4.4 Hz, 1H), 7.30 (d, J=8.7 Hz, 2H), 7.18-7.08 (m, 5H), 6.91-6.85 (m, 3H), 5.11-5.04 (m, 2H), 4.57 (s, 2H), 4.07-4.03 (m, 8H), 3.67-3.65 (m, 3H), 3.33 (br, 3H), 3.21-3.07 (m, 5H), 3.95-2.86 (br, 1H), 2.66-2.56 (m, 1H), 2.43-2.33 (m, 1H), 2.18-2.12 (m, 2H), 1.99-1.93 (m, 1H), 1.42 (d, J=7.0 Hz, 3H).

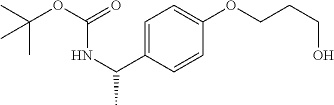

Compound 2. (S)-tert-butyl 1-(4-(3-hydroxypropoxy)phenyl)ethylcarbamate. A mixture of compound 1 (2.5 mmol, 1 eq), bromopropanol (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 2. ¹H NMR (400 MHz, DMSO-d₆) δ 7.24 (d, J=6.2 Hz, 1H), 7.19 (d, J=7.4 Hz, 2H), 6.85 (d, J=7.2 Hz, 2H), 7.36-7.27 (m, 5H), 4.52 (t, J=6.2 Hz, 1H), 4.01-3.98 (m, 2H), 3.56-3.52 (m, 2H), 3.46-3.39 (m, 1H), 1.86-1.82 (m, 2H), 1.35 (s, 9H), 1.25 (d, J=6.7 Hz, 3H).

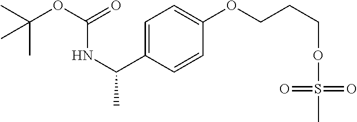 [SMILES: C[C@H](NC(=O)OC(C)(C)C)c1ccc(OCCCOS(C)(=O)=O)cc1]

Compound 3. (S)-3-(4-(1-(tert-butoxycarbonylamino)ethyl)phenoxy)propyl methanesulfonate. A solution of compound 2 (161 mg, 0.546 mmol) in CH₂Cl₂ was mixed with 0.1 mL of DIPEA (0.819 mmol) and cooled to 0° C. 75 mg of MsCl (0.655 mmol) was added dropwise. The mixture was stirred at room temperature for 2 hours, washed with water, dried over Na₂SO₄ and evaporated. The product was used without purification. Yield 203 mg (98%).

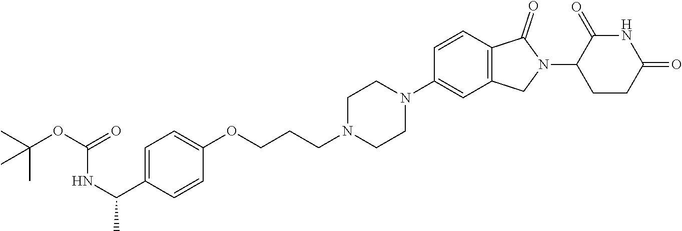 [SMILES: C[C@H](NC(=O)OC(C)(C)C)c1ccc(OCCCN2CCN(c3ccc4c(c3)CN(C3CCC(=O)NC3=O)C4=O)CC2)cc1]

Compound. 4 tert-butyl (1S)-1-(4-(3-(4-(2-(2,6-dioxopiperidin-3-yl)-1-oxoisoindolin-5-yl)piperazin-1-yl)propoxy)phenyl)ethylcarbamate. A solution of A (310 mg, 0.831 mmol), compound 3 (302 mg, 0.831 mmol), DIPEA (214 mg, 1.66 mmol), KI (207 mg, 1.25 mmol) and tetrabuthyl ammonium iodide (26 mg, 0.08 mmol) in 5 mL of DMSO was stirred for an 48 hour at 50° C. The product was extracted with dichloromethane (3 by 10 ml). After extraction, the combined organic layer was dried over Na₂SO₄. Purification by silica gel column chromatography (gradient ethyl acetate/tetrahydrofuran=100:1 to 1:100 as a developing solvent) afforded compound 4 (152 mg, 30%). LCMS (C18 column 100×4.6 mm, 5.0 μm, pore size 100 Å, water-acetonitrile+0.1% TFA, gradient 5 to 87% for 10 min, retention time 4.84 min). MS (ESI) m/z 606.4 [MH]+.

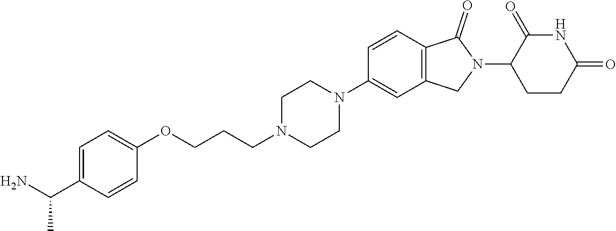 [SMILES: C[C@H](N)c1ccc(OCCCN2CCN(c3ccc4c(c3)CN(C3CCC(=O)NC3=O)C4=O)CC2)cc1]

Compound 5. 3-(5-(4-(3-(4-((S)-1-aminoethyl)phenoxy)propyl)piperazin-1-yl)-1-oxoisoindolin-2-yl)piperidine-2,6-dione. A solution of compound 4 (0.248 mmol, 1 eq) in dry DCM (50 mL) with 0.5 ml 3M HCl in dioxane in dioxane (5 eq) were stirred for 24 h at r.t. The mixture was evaporated to dryness to give the crude product 4 which was used in the next step without additional purification Yield 98% LCMS (C18 column 100×4.6 mm, 5.0 μm, pore size 100 Å, water-acetonitrile+0.1% TFA, gradient 5 to 87% for 10 min, retention time 3.39 min). MS (ESI) m/z 506.6 [MH]+.

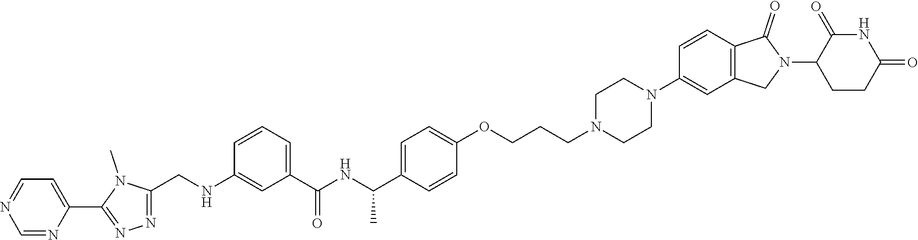 [SMILES: C[C@H](NC(=O)c1cccc(NCc2nnc(-c3ccncn3)n2C)c1)c1ccc(OCCCN2CCN(c3ccc4c(c3)CN(C3CCC(=O)NC3=O)C4=O)CC2)cc1]

D118. N-((1S)-1-(4-(3-(4-(2-(2,6-dioxopiperidin-3-yl)-1-oxoisoindolin-5-yl)piperazin-1-yl)propoxy)phenyl)ethyl)-3-((4-methyl-5-(pyrimidin-4-yl)-4H-1,2,4-triazol-3-yl)methylamino)benzamide hydrochloride. A mixture of compound 5 (0.1 mmol, 1 eq), compound B (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile) Yield 47%. LCMS (C18 column 100×4.6 mm, 5.0 μm, pore size 100 Å, water-acetonitrile+0.1% TFA, gradient 5 to 87% for 10 min, retention time 4.40 min). MS (ESI) m/z 798.1 [MH]+. ¹H NMR (400 MHz, DMSO-d₆) δ 10.95 (s, 1H), 9.59 (br, 1H), 9.33 (s, 1H), 8.98 (d, J=5.3 Hz, 1H), 8.55 (d, J=8.2 Hz, 1H), 8.16 (dd, J=6.6, 1.4 Hz, 1H), 7.60 (d, J=4.4 Hz, 1H), 7.30 (d, J=8.7 Hz, 2H), 7.18-7.08 (m, 5H), 6.91-6.85 (m, 3H), 5.11-5.04 (m, 2H), 4.57 (s, 2H), 4.07-4.03 (m, 8H), 3.67-3.65 (m, 3H), 3.33 (br, 3H), 3.21-3.07 (m, 5H), 3.95-2.86 (br, 1H), 2.66-2.56 (m, 1H), 2.43-2.33 (m, 1H), 2.18-2.12 (m, 2H), 1.99-1.93 (m, 1H), 1.42 (d, J=7.0 Hz, 3H).

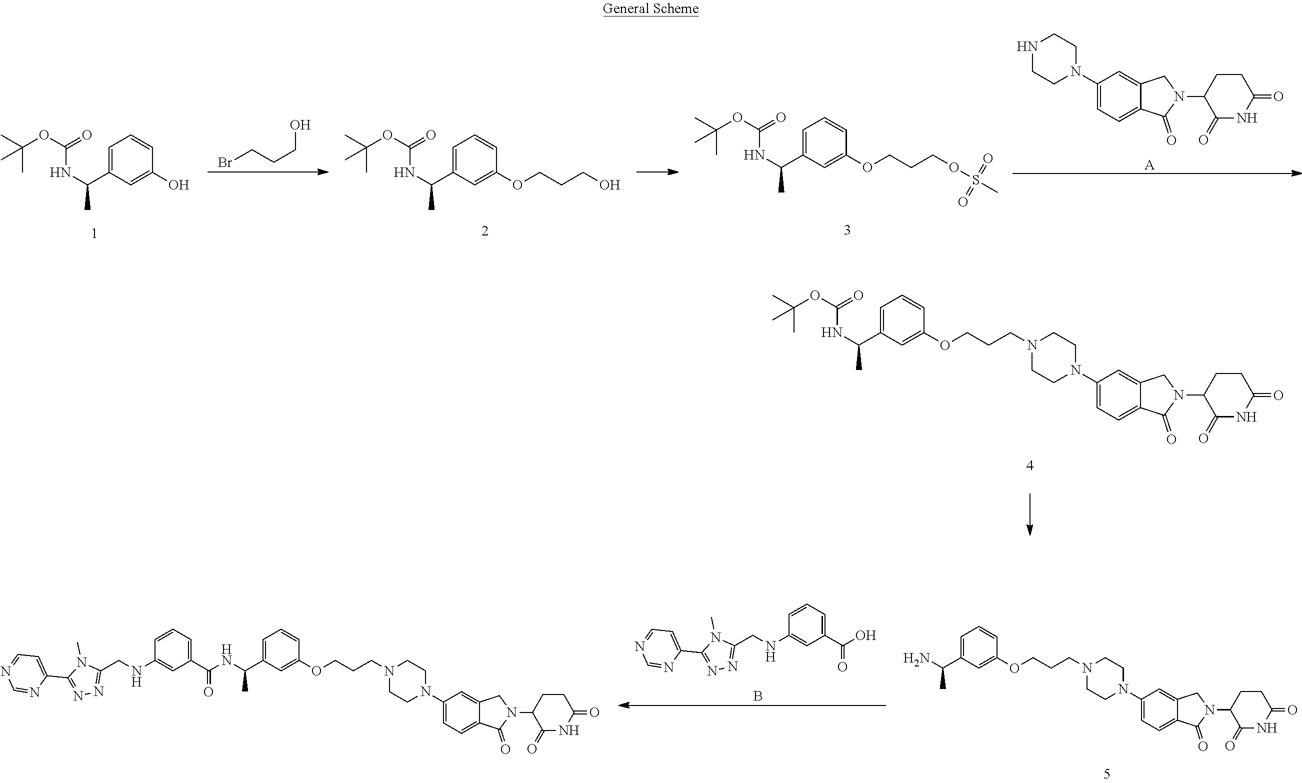

### Compounds, Procedures, and Analytical Data

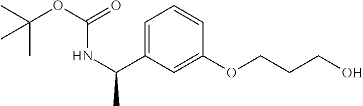 [SMILES: C[C@@H](NC(=O)OC(C)(C)C)c1cccc(OCCCO)c1]

Compound 2. (R)-tert-butyl 1-(3-(3-hydroxypropoxy)phenyl)ethylcarbamate. A mixture of compound 1 (500 mg, 2.1 mmol, 1 eq), bromopropanol (587 mg, 4.22 mmol, 2 eq), K₂CO₃ (1.18 g, 8.44 mmol, 4 eq) and anh. acetonitrile (50 mL) was stirred and heated under reflux for 12 h, filtered, and concentrated under reduced pressure. The residue was purified by silica gel column chromatography using ethyl acetate and hexane (1:1) as an eluent to afford the desired product 2. Yield 82%. ¹H NMR (400 MHz, DMSO-d₆) δ 7.29 (d, J=7.0 Hz, 1H), 7.19 (t, J=8.5 Hz, 2H), 6.84-6.83 (m, 2H), 6.74 (dd, J=8.4, 1.5 Hz, 1H), 4.59-4.52 (m, 2H), 4.01 (t, J=5.1 Hz, 1H), 3.58-3.53 (m, 2H), 1.88-1.82 (m, 2H), 1.36 (s, 9H), 1.28 (d, J=7.0 Hz, 3H).

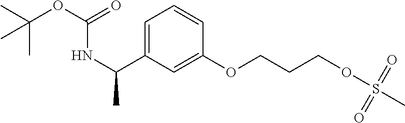 [SMILES: C[C@@H](NC(=O)OC(C)(C)C)c1cccc(OCCCOS(C)(=O)=O)c1]

Compound 3. (R)-3-(3-(1-(tert-butoxycarbonylamino)ethyl)phenoxy)propyl methanesulfonate. Solution of compound 2 (161 mg, 0.546 mmol) in CH₂Cl₂ was mixed with 0.1 mL of DIPEA (0.819 mmol) and cooled to 0° C. 75 mg of MsCl (0.655 mmol) was added dropwise. The mixture was stirred at room temperature for 2 hours, washed with water, dried over Na₂SO₄ and evaporated. The product was used without purification. Yield 203 mg (98%).

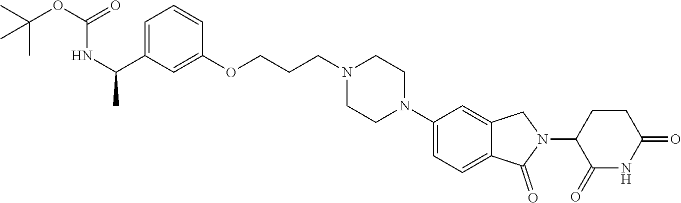 [SMILES: C[C@@H](NC(=O)OC(C)(C)C)c1cccc(OCCCN2CCN(c3ccc4c(c3)CN(C3CCC(=O)NC3=O)C4=O)CC2)c1]

Compound 4. Tert-butyl (1R)-1-(3-(3-(4-(2-(2,6-dioxopiperidin-3-yl)-1-oxoisoindolin-5-yl)piperazin-1-yl)propoxy)phenyl)ethylcarbamate. Solution of A (146 mg, 0.402 mmol), compound 3 (150 mg, 0.402 mmol), DIPEA (104 mg, 0.80 mmol), KI (100 mg, 0.603 mmol) and tetrabuthyl ammonium iodide (13 mg, 0.04 mmol) in 5 mL of DMSO were stirred for an 48 hour at 50° C. The product was extracted with dichloromethane (3 by 10 ml). After extraction, the combined organic layer was dried over Na₂SO₄. Purification by silica gel column chromatography (gradient ethyl acetate/tetrahydrofuran=100:1 to 1:100 as a developing solvent) afforded compound 4 (90 mg, 37%). LCMS (C18 column 100×4.6 mm, 5.0 μm, pore size 100 Å, water-acetonitrile+0.1% TFA, gradient 5 to 87% for 10 min, retention time 4.86 min). MS (ESI) m/z 606.3 [MH]+.

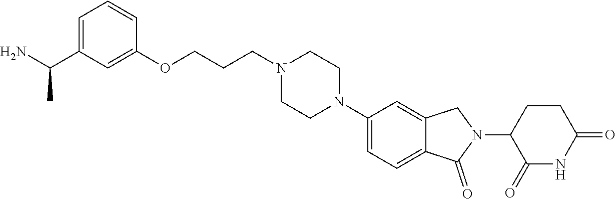 [SMILES: C[C@@H](N)c1cccc(OCCCN2CCN(c3ccc4c(c3)CN(C3CCC(=O)NC3=O)C4=O)CC2)c1]

Compound 5. 3-(5-(4-(3-(3-((R)-1-aminoethyl)phenoxy)propyl)piperazin-1-yl)-1-oxoisoindolin-2-yl)piperidine-2,6-dione hydrochloride. A solution of compound 4 90 mg, 0.148 mmol) in dry DCM (50 mL) with 0.3 ml 3M HCl in dioxane in dioxane (5 eq) were stirred for 24 h at r.t. The mixture was evaporated to dryness to give the crude product 5 which was used in the next step without additional purification. Yield 87 mg (98%). LCMS (C18 column 100×4.6 mm, 5.0 μm, pore size 100 Å, water-acetonitrile+0.1% TFA, gradient 5 to 87% for 10 min, retention time 3.73 min). MS (ESI) m/z 506.6 [MH]+

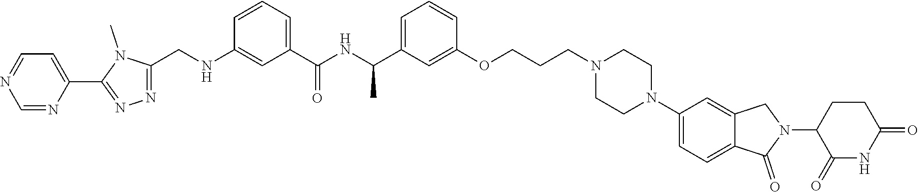 [SMILES: C[C@@H](NC(=O)c1cccc(NCc2nnc(-c3ccncn3)n2C)c1)c1cccc(OCCCN2CCN(c3ccc4c(c3)CN(C3CCC(=O)NC3=O)C4=O)CC2)c1]

D123. N-((1R)-1-(3-(3-(4-(2-(2,6-dioxopiperidin-3-yl)-1-oxoisoindolin-5-yl)piperazin-1-yl)propoxy)phenyl)ethyl)-3-((4-methyl-5-(pyrimidin-4-yl)-4H-1,2,4-triazol-3-yl)methylamino)benzamide hydrochloride. A mixture of compound 5 (0.1 mmol, 1 eq), compound B (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was treated with 3M HCl in dioxane, evaporated to dryness.

The residue was purified by HPLC in acidic conditions (YMC-Pack ODS-AQ 250×20 mml, S-10 μm, 12 nm, gradient water-acetonitrile) Yield 31%. LCMS (C18 column 100×4.6 mm, 5.0 μm, pore size 100 Å, water-acetonitrile+0.1% TFA, gradient 5 to 87% for 10 min, retention time 4.43 min). MS (ESI) m/z 798.5 [MH]+¹H NMR (400 MHz, CDCl₃) δ 10.94 (s, 1H), 9.62 (br, 1H), 9.33 (s, 1H), 8.96 (d, J=5.3 Hz, 1H), 8.57 (d, J=8.2 Hz, 1H), 8.16 (dd, J=6.6, 1.4 Hz, 1H), 7.60 (d, J=4.4 Hz, 1H), 7.25 (t, J=7.9 Hz, 1H), 7.20-7.10 (m, 5H), 7.00-6.95 (m, 2H), 6.89 (dd, J=7.8, 1.1 Hz, 1H), 6.80 (dd, J=7.9, 1.9 Hz, 1H), 5.12-5.04 (m, 2H), 4.57 (s, 2H), 4.38-4.22 (m, 2H), 4.07-4.02 (m, 6H), 3.67-3.65 (m, 2H), 3.34 (br, 3H), 3.23-3.06 (m, 5H), 2.94-2.86 (m, 1H), 2.66-2.56 (m, 1H), 2.43-2.33 (m, 1H), 2.20-2.13 (m, 2H), 2.01-1.92 (m, 1H), 1.46 (d, J=7.0 Hz, 3H).

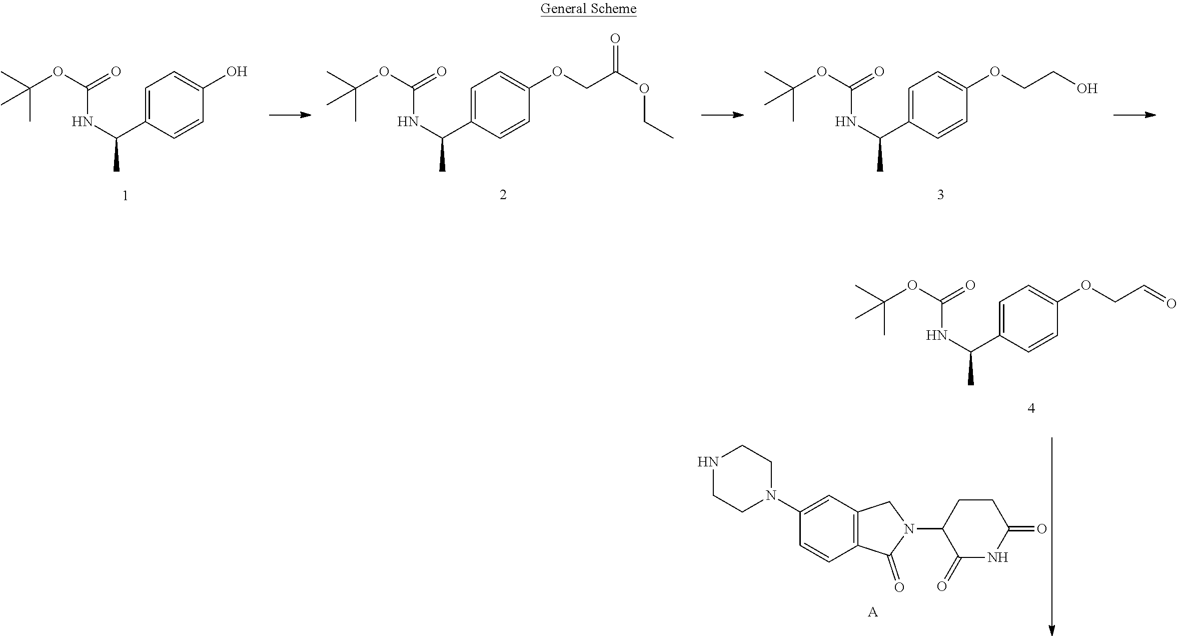

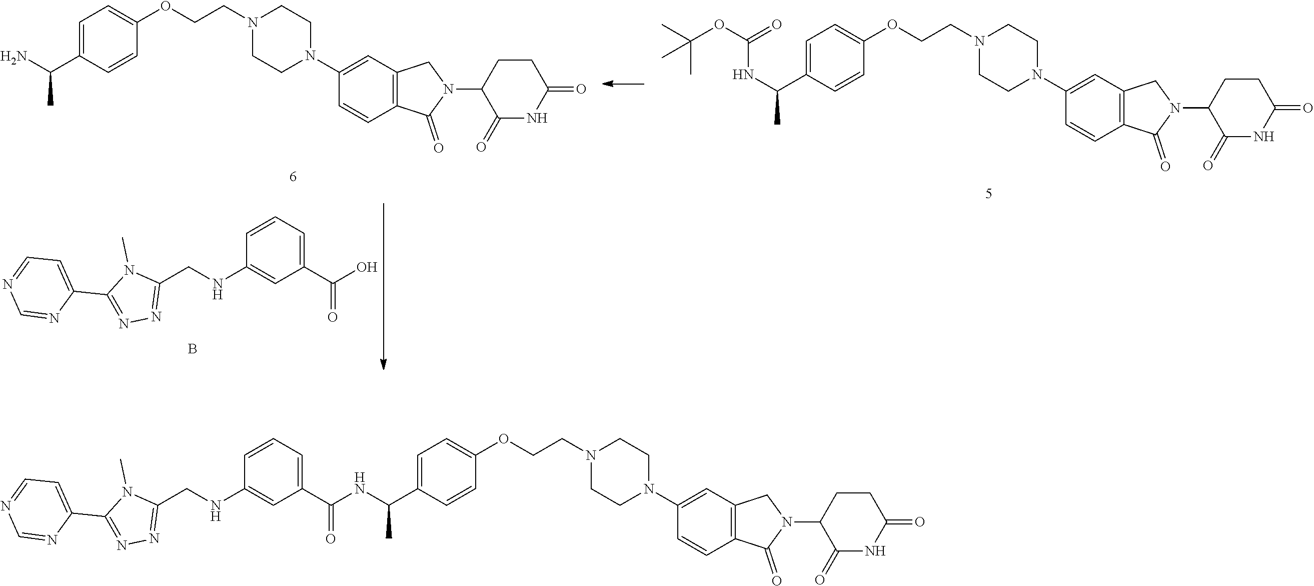

### Compounds, Procedures, and Analytical Data

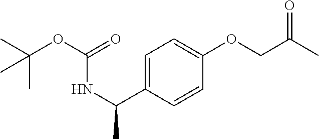 [SMILES: CC(=O)COc1ccc([C@@H](C)NC(=O)OC(C)(C)C)cc1]

Compound 2 (R)-ethyl 2-(4-(1-(tert-butoxycarbonylamino)ethyl)phenoxy)acetate. A mixture of compound 1 (200 mg, 0.45 mmol), ethyl chloroacetate (154 mg, 1.27 mmol), K₂CO₃ (354 g, 2.53 mmol) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 270 mg (94%) of the desired product 2. ¹H NMR (400 MHz, DMSO-d₆) δ 7.26 (d, J=7.0 Hz, 1H), 7.18 (d, J=8.5 Hz, 2H), 6.85 (d, J=8.5 Hz, 2H), 4.72 (s, 2H), 4.52 (m, 1H), 4.18 (q, J=7.1 Hz, 2H), 1.35 (s, 9H), 1.27 (d, J=7.0 Hz, 3H), 1.20 (t, J=7.1 Hz, 3H).

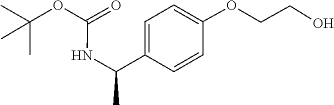 [SMILES: C[C@@H](NC(=O)OC(C)(C)C)c1ccc(OCCO)cc1]

Compound 3. (R)-tert-butyl 1-(4-(2-hydroxyethoxy)phenyl)ethylcarbamate. To a mixture of compound 2 (200 mg, 0.593 mmol) in Et₂O (5 mL), LiBH₄ (20 mg, 0.928 mmol) was added, followed by methanol (30 mg, 0.928 mmol) at 0° C. The reaction was stirred at ambient temperature overnight. The solvent was evaporated and the residue was partitioned between DCM (5 ml) and water, contains with citric acid (0.7 g, 3.64 mmol in water 1 ml). The extract was separated and dried over Na₂SO₄. The crude product 3 was used further without additional purification. Yield (170 mg, 95%). Colorless oil LCMS (C18 column 20×2 mm, 2.5 μm, pore size 100 Å, water-acetonitrile+0.1% TFA, gradient 5 to 87% for 3 min, retention time 1.32 min). MS (ESI) m/z 282.8 [MH]+¹H NMR (400 MHz, DMSO-d₆) δ 7.27 (d, J=7.0 Hz, 1H), 7.17 (d, J=8.5 Hz, 2H), 6.84 (d, J=8.5 Hz, 2H), 4.83 (t, J=5.5 Hz, 1H), 4.52 (m, 1H), 3.94 (t, J=4.9 Hz, 2H), 3.71-3.67 (m, 2H), 1.35 (s, 9H), 1.26 (d, J=7.0 Hz, 3H).

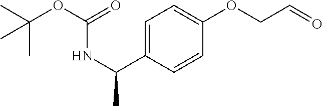 [SMILES: C[C@@H](NC(=O)OC(C)(C)C)c1ccc(OCC=O)cc1]

Compound 4. (R)-tert-butyl 1-(4-(2-oxoethoxy)phenyl)ethylcarbamate. A flask was charged with 10 mL of dichloromethane and oxalyl chloride (0.242 g, 1.91 mmol). The solution was stirred and cooled to −60° C. then dimethyl sulfoxide (0.149 g, 1.91 mmol) in 5 mL of dichloromethane was added dropwise at a rapid rate. After 5 min compound 3 (0.139 g, 0.64 mmol) in 5 ml of dichloromethane was added dropwise maintaining the temperature at −50 to −60° C. After another 15 min, triethylamine (1.0 ml, 7.0 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 0.140 g (95%) of crude product 4 that used further without additional purification.

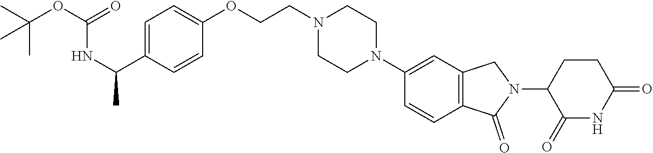 [SMILES: C[C@@H](NC(=O)OC(C)(C)C)c1ccc(OCCN2CCN(c3ccc4c(c3)CN(C3CCC(=O)NC3=O)C4=O)CC2)cc1]

Compound 5. tert-butyl (1R)-1-(4-(4-(4-(2-(2,6-dioxopiperidin-3-yl)-1-oxoisoindolin-5-yl)piperazin-1-yl)butoxy)phenyl)ethylcarbamate. To a suspension of 3-(1-oxo-5-(piperazin-1-yl)isoindolin-2-yl)piperidine-2,6-dione hydrochloride (A) (0.180 g, 0.46 mmol) and compound 4 (0.138 g, 0.46 mmol) in 10 mL of DCM was added DIPEA (0.15 mL, 0.92 mmol) and the mixture was stirred for an hour. Then sodium triacetoxyborohydride (0.415 g, 1.84 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 5 (0.241 g, 83%). LCMS (C18 column 100×4.6 mm, 5.0 μm, pore size 100 Å, water-acetonitrile+0.1% TFA, gradient 5 to 87% for 10 min, retention time 4.71 min). MS (ESI) m/z 592.7 [MH]+

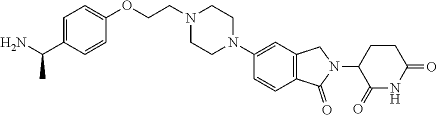 [SMILES: C[C@@H](N)c1ccc(OCCN2CCN(c3ccc4c(c3)CN(C3CCC(=O)NC3=O)C4=O)CC2)cc1]

Compound 6. 3-(5-(4-(2-(4-((R)-1-aminoethyl)phenoxy)ethyl)piperazin-1-yl)-1-oxoisoindolin-2-yl)piperidine-2,6-dione hydrochloride. A solution of compound 5 (0.43 mmol, 1 eq) in dry DCM (50 mL) with 0.8 ml 3 M HCl in dioxane (5 eq) were stirred for 24 h at r.t. The mixture was evaporated to dryness to give the crude product 6 which was used in the next step without additional purification Yield 87% LCMS (C18 column 100×4.6 mm, 5.0 μm, pore size 100 Å, water-acetonitrile+0.1% TFA, gradient 5 to 87% for 10 min, retention time 3.26 min). MS (ESI) m/z 492.2 [MH]+

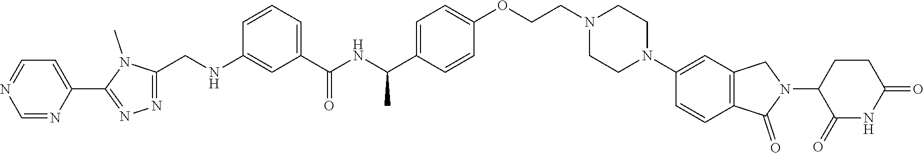 [SMILES: C[C@@H](NC(=O)c1cccc(NCc2nnc(-c3ccncn3)n2C)c1)c1ccc(OCCN2CCN(c3ccc4c(c3)CN(C3CCC(=O)NC3=O)C4=O)CC2)cc1]

D174. N-((1R)-1-(4-(2-(4-(2-(2,6-dioxopiperidin-3-yl)-1-oxoisoindolin-5-yl)piperazin-1-yl)ethoxy)phenyl)ethyl)-3-((4-methyl-5-(pyrimidin-4-yl)-4H-1,2,4-triazol-3-yl)methylamino)benzamide hydrochloride. A mixture of compound 6 (150 mg, 0.266 mmol, 1 eq), compound B (82 mg, 0.266 mmol, 1 eq), TBTU (128 mg, 0.399 mmol, 2 eq), DIPEA (137 mg, 1.02 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile). Yield 54%. LCMS (C18 column 100×4.6 mm, 5.0 μm, pore size 100 Å, water-acetonitrile+0.1% TFA, gradient 5 to 87% for 10 min, retention time 4.31 min). MS (ESI) m/z 784.6 [MH]+. ¹H NMR (400 MHz, DMSO-d₆) δ 10.97 (s, 1H), 9.92 (br, 1H), 9.33 (s, 1H), 8.96 (d, J=5.3 Hz, 1H), 8.56 (d, J=8.2 Hz, 1H), 8.17 (dd, J=6.6, 1.4 Hz, 1H), 7.60 (d, J=4.4 Hz, 1H), 7.36 (d, J=8.7 Hz, 2H), 7.18-7.08 (m, 5H), 6.99 (d, J=8.5 Hz, 2H), 6.88 (d, J=9.2 Hz, 1H), 5.12-5.04 (m, 2H), 4.57 (s, 2H), 4.37-4.21 (m, 5H), 4.07-3.97 (m, 5H), 3.68 (br, 3H), 3.29 (br, 3H), 3.22 (br, 2H), 2.94-2.85 (m, 1H), 2.66-2.56 (m, 1H), 2.43-2.33 (m, 1H), 2.00-1.94 (m, 1H), 1.45 (d, J=7.0 Hz, 3H).

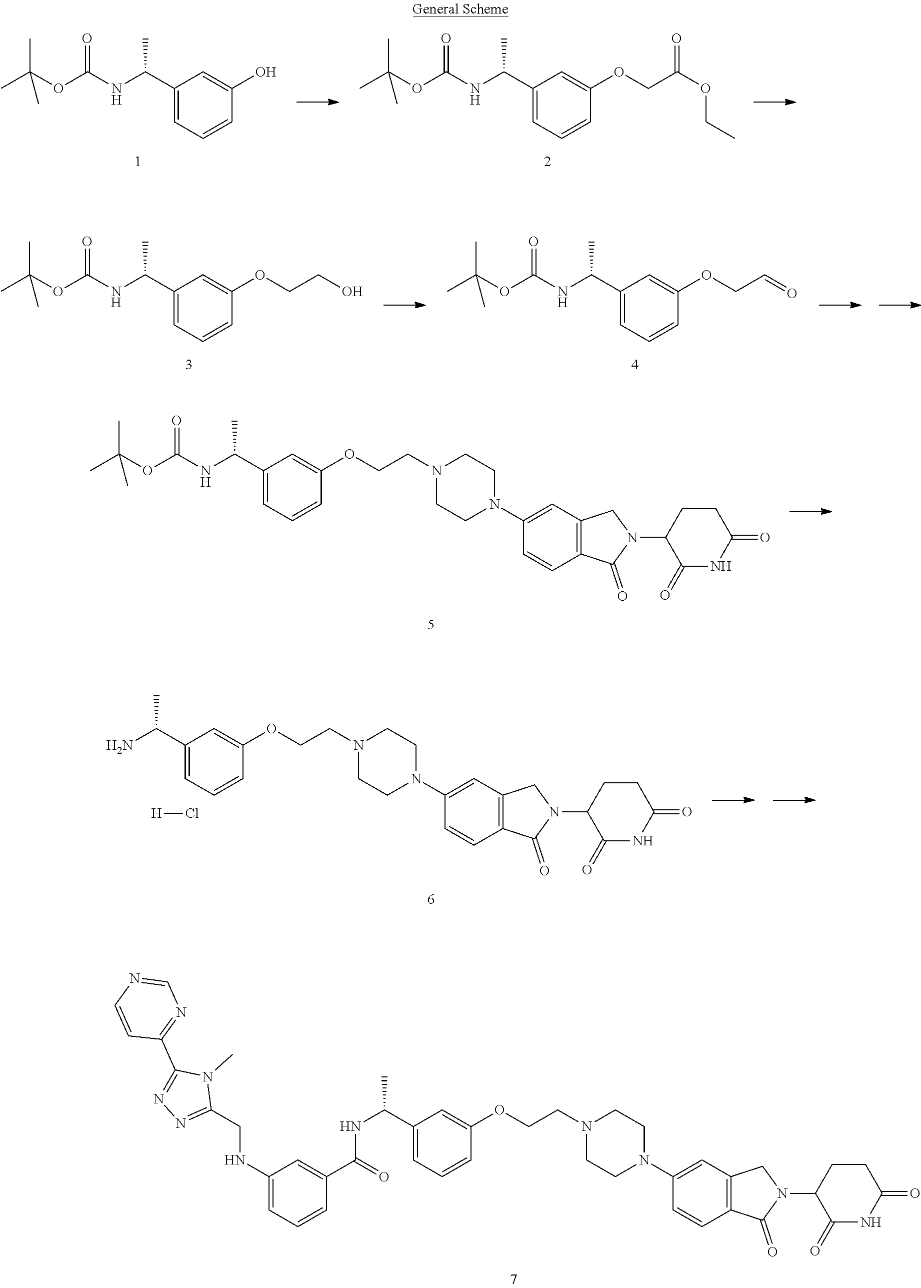

### Compounds, Procedures, and Analytical Data

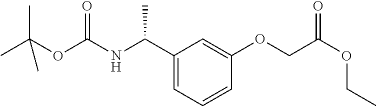 [SMILES: CCOC(=O)COc1cccc([C@@H](C)NC(=O)OC(C)(C)C)c1]

Compound 2. (R)-ethyl 2-(3-(1-(tert-butoxycarbonylamino)ethyl)phenoxy)acetate. To a solution of phenol 1 (0.42 g, 1.77 mmol) and ethyl chloroacetate (0.25 ml, 0.28 g, 2.33 mmol) in MeCN (5 ml), K2CO3 (0.8 g, 5.8 mmol) was added and the reaction mixture was refluxed with stirring for 48 hours. Then the solvent was evaporated and the residue was partitioned between methylene chloride (5 ml) and water (5 ml). The organic layer was separated; aqueous one was extracted with methylene chloride (5 ml). Combined organic layers were dried over MgSO4 and concentrated. The product 2 was chromatographed on silica, using chloroform and then a mixture chloroform-methanol (100 to 1) as an eluent. Yield 0.54 g, (94%).

Colorless oil 94%. LCMS (C18 column 20×2 mm, 2.5 μm, pore size 100 Å, water-acetonitrile+0.1% TFA, gradient 5 to 87% for 3 min, retention time 1.61 min). MS (ESI) m/z 324.3 [MH]+. ¹H NMR (400 MHz, CDCl₃) δ 7.26 (t, J=7.6 Hz, 1H), 6.95 (d, J=7.6 Hz, 1H), 6.89 (s, 1H), 6.78 (dd, J=8.2, 2.4 Hz, 1H), 4.76 (s, 1H), 4.62 (s, 2H), 4.29 (q, J=7.2 Hz, 2H), 1.43 (s, 12H), 1.30 (t, J=7.2 Hz, 3H).

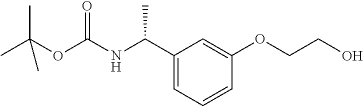 [SMILES: C[C@@H](NC(=O)OC(C)(C)C)c1cccc(OCCO)c1]

Compound 3. (R)-tert-butyl 1-(3-(2-hydroxyethoxy)phenyl)ethylcarbamate. To a mixture of compound 2 (0.6 g, 1.85 mmol) in Et2O (5 mL), LiBH4 (0.06 g, 2.75 mmol) was added, followed by methanol (0.11 ml, 0.087 g, 2.75 mmol) at 0° C. The reaction was stirred at ambient temperature overnight. The solvent was evaporated and the residue was partitioned between DCM (5 ml) and water, contains with citric acid (0.7 g, 3.64 mmol in water 1 ml). The extract was separated and dried. The crude product 3 was used further without additional purification. Yield (0.5 g, 97%). Colorless oil 97%. LCMS (C18 column 20×2 mm, 2.5 μm, pore size 100 Å, water-acetonitrile+0.1% TFA, gradient 5 to 87% for 3 min, retention time 1.33 min). MS (ESI) m/z 282.6 [MH]+. ¹H NMR (400 MHz, CDCl₃) δ 7.26 (t, J=7.6 Hz, 1H), 6.92 (d, J=7.6 Hz, 1H), 6.88 (s, 1H), 6.81 (d, J=8.2 Hz, 1H), 4.78 (s, 3H), 4.19-4.04 (m, 2H), 4.01-3.91 (m, 2H), 1.44 (d, J=6.6 Hz, 12H).

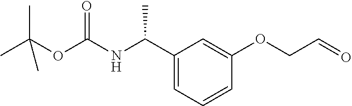 [SMILES: C[C@@H](NC(=O)OC(C)(C)C)c1cccc(OCC=O)c1]

Compound 4. (R)-tert-butyl 1-(3-(2-oxoethoxy)phenyl)ethylcarbamate. A flask was charged with 10 mL of dichloromethane and (0.1 ml, 0.145 g, 0.11 mmol) of oxalyl chloride. The solution was stirred and cooled at −50 to −60° C. as (0.12 ml, 0.132 g, 0.17 mmol) of dimethyl sulfoxide in 10 mL of dichloromethane was added dropwise at a rapid rate. After 5 min compound 3 (0.2 g, 0.71 mmol) in 5 ml of dichloromethane was added dropwise maintaining the temperature at −50 to −60° C. After another 15 min, 0.5 mL of triethylamine (3.59 mmol) was added dropwise while keeping the temperature at or below −50° C. Stirring was continued for 5 min, after which time the mixture was allowed to warm to room temperature and 20 mL of water was added. The aqueous layer was separated and extracted with two 10-ml portions of dichloromethane. The organic layers were combined and dried over anhydrous magnesium sulfate. The filtered solution was concentrated to afford 0.19 g (95%) of crude product 4 that used further without additional purification.

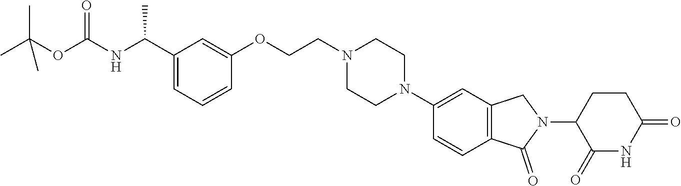 [SMILES: C[C@@H](NC(=O)OC(C)(C)C)c1cccc(OCCN2CCN(c3ccc4c(c3)CN(C3CCC(=O)NC3=O)C4=O)CC2)c1]

Compound 5. tert-butyl (1R)-1-(3-(2-(4-(2-(2,6-dioxopiperidin-3-yl)-1-oxoisoindolin-5-yl)piperazin-1-yl)ethoxy)phenyl)ethylcarbamate. To a suspension of 3-(1-oxo-5-(piperazin-1-yl)isoindolin-2-yl)piperidine-2,6-dione hydrochloride (0.26 g, 0.71 mmol) in 10 mL of DCM was added DIPEA (0.25 mL, 1.44 mmol) and the mixture was stirred for an hour. Then compound 4 (0.2 g, 0.71 mmol) and sodium triacetoxyborohydride (0.46 g, 2.17 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 5 (0.36 g, 84%). LCMS (C18 column 20×2 mm, 2.5 μm, pore size 100 Å, water-acetonitrile+0.1% TFA, gradient 5 to 87% for 3 min, retention time 1.17 min). MS (ESI) m/z 592.7 [MH]+. ¹H NMR (400 MHz, CDCl₃) δ 8.25 (d, J=5.9 Hz, 1H), 7.74 (d, J=8.6 Hz, 1H), 7.25 (d, J=8.6 Hz, 1H), 7.00 (d, J=8.2 Hz, 1H), 6.92-6.86 (m, 4H), 6.81 (d, J=8.0 Hz, 1H), 5.19 (dd, J=13.1, 5.0 Hz, 1H), 4.81-4.75 (m, 1H), 4.41 (d, J=15.7 Hz, 1H), 4.26 (d, J=15.6 Hz, 1H), 4.18 (t, J=5.0 Hz, 2H), 3.41-3.34 (m, 4H), 2.98-2.84 (m, 3H), 2.82-2.77 (m, 4H), 2.42-2.24 (m, 1H), 2.22-2.15 (m, 1H), 1.46-1.38 (m, 12H).

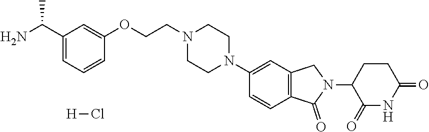 [SMILES: C[C@@H](N)c1cccc(OCCN2CCN(c3ccc4c(c3)CN(C3CCC(=O)NC3=O)C4=O)CC2)c1 ; Cl]

Compound 6. 3-(5-(4-(2-(3-((R)-1-aminoethyl)phenoxy)ethyl)piperazin-1-yl)-1-oxoisoindolin-2-yl)piperidine-2,6-dione hydrochloride. To an ice cold solution of compound 5 (0.26 g, 0.44 mmol) in methylene chloride (5 ml), dioxanic HCl solution (1.5 ml, 3M) was added and the mixture was stirred at room temperature for overnight. Then the solvent was evaporated, chloroform (5 ml) was added and evaporated again. The residue was crude 6 and used further. Yield 0.22 g (97%). Colorless crystals 97%. LCMS (C18 column 20×2 mm, 2.5 μm, pore size 100 Å, water-acetonitrile+0.1% TFA, gradient 5 to 87% for 3 min, retention time 0.84 min). MS (ESI) m/z 492.5 [MH]+.

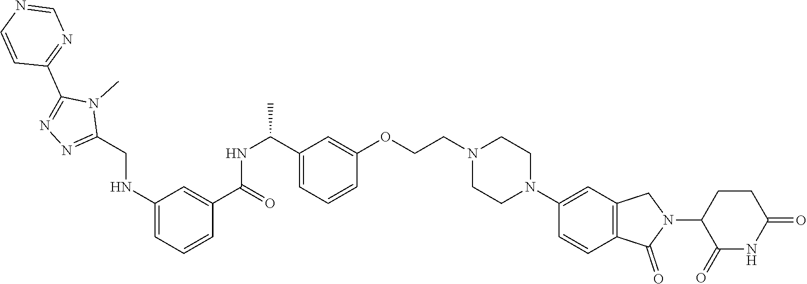 [SMILES: C[C@@H](NC(=O)c1cccc(NCc2nnc(-c3ccncn3)n2C)c1)c1cccc(OCCN2CCN(c3ccc4c(c3)CN(C3CCC(=O)NC3=O)C4=O)CC2)c1]

Compound 7. D128. N-((1R)-1-(3-(2-(4-(2-(2,6-dioxopiperidin-3-yl)-1-oxoisoindolin-5-yl)piperazin-1-yl)ethoxy)phenyl)ethyl)-3-((4-methyl-5-(pyrimidin-4-yl)-4H-1,2,4-triazol-3-yl)methylamino)benzamide. To an ice cold suspension of amine hydrochloride 6 (0.11 g, 0.22 mmol) in methylene chloride (5 ml), 3-((4-methyl-5-(pyrimidin-4-yl)-4H-1,2,4-triazol-3-yl)methylamino)benzoic acid (0.07 g, 0.24 mmol) was added, followed by hydroxyl benzotriazole hydrate (0.05 g, 0.33 mmol) and EDCI (0.06 g, 0.33 mmol) and finally DIPEA (0.4 ml, 0.3 g, 2.31 mmol). The reaction mixture was stirred at ambient temperature overnight. Then water (5 ml) was added and organic layer was separated. Aqueous layer was extracted with methylene chloride (5 ml). Combined organic layers were dried over MgSO4 and evaporated. The residue was treated with dioxanic HCl (1 ml, 3M), and after concentration, was purified by HPLC chromatography. Yield of product 7 was 0.094 g (53%). LCMS (C18 column 20×2 mm, 2.5 μm, pore size 100 Å, water-acetonitrile+0.1% TFA, gradient 5 to 87% for 10 min, retention time 4.30 min). MS (ESI) m/z 784.9 [MH]+. ¹H NMR (400 MHz, DMSO-d₆) δ 10.93 (s, 1H), 9.87 (s, 1H), 9.33 (s, 1H), 8.96 (d, J=5.3 Hz, 1H), 8.58 (d, J=8.1 Hz, 1H), 8.16 (d, J=5.3 Hz, 1H), 7.59 (d, J=8.4 Hz, 1H), 7.29 (t, J=7.8 Hz, 1H), 7.23-7.09 (m, 5H), 7.04 (d, J=9.4 Hz, 2H), 6.89 (t, J=6.5 Hz, 2H), 5.19-4.96 (m, 2H), 4.58 (s, 2H), 4.37-4.33 (m, 2H), 4.24 (d, J=16.7 Hz, 1H), 4.07 (s, 3H), 4.04-3.95 (m, 2H), 3.63 (s, 4H), 3.31-3.18 (m, 5H), 2.98-2.83 (m, 1H), 2.70-2.55 (m, 1H), 2.45-2.29 (m, 1H), 1.97 (s, 1H), 1.46 (d, J=7.1 Hz, 3H).

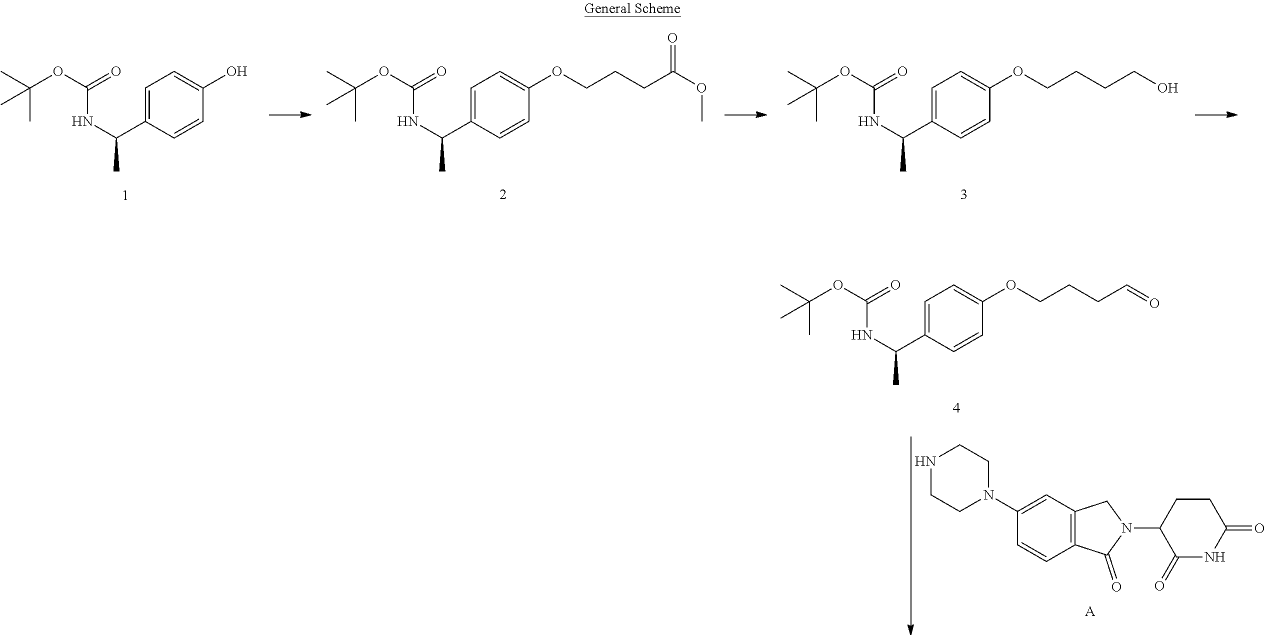

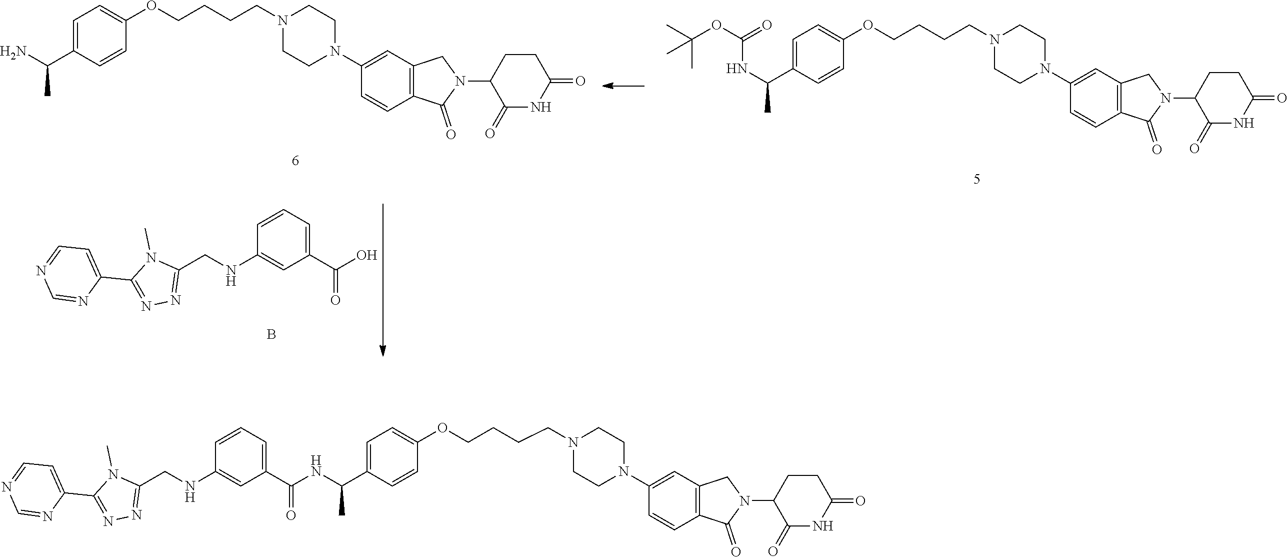

### Compounds, Procedures, and Analytical Data

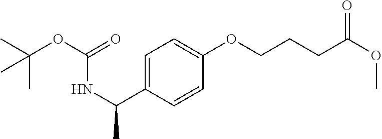 [SMILES: COC(=O)CCCOc1ccc([C@@H](C)NC(=O)OC(C)(C)C)cc1]

Compound 2. (R)-methyl 4-(4-(1-(tert-butoxycarbonylamino)ethyl)phenoxy)butanoate. A mixture of compound 1 (200 mg, 0.45 mmol), methyl bromobutanoate (228 mg, 1.27 mmol), K₂CO₃ (354 g, 2.53 mmol) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 280 mg (94%) of the desired product 2. ¹H NMR (400 MHz, DMSO-d₆) δ 7.24 (d, J=7.0 Hz, 1H), 7.17 (d, J=8.5 Hz, 2H), 6.84 (d, J=8.5 Hz, 2H), 4.54 (t, J=6.5 Hz, 1H), 3.94 (t, J=6.3 Hz, 2H), 3.60 (s, 3H), 2.45 (t, J=7.3 Hz, 2H), 1.97-1.92 (m, 2H), 1.35 (s, 9H), 1.25 (d, J=7.0 Hz, 3H).

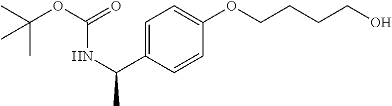 [SMILES: C[C@@H](NC(=O)OC(C)(C)C)c1ccc(OCCCCO)cc1]

Compound 3. (R)-tert-butyl 1-(4-(4-hydroxybutoxy)phenyl)ethylcarbamate. To a mixture of compound 2 (200 mg, 0.593 mmol) in Et₂O (5 mL), LiBH₄ (19 mg, 0.889 mmol) was added, followed by methanol (28 mg, 0.889 mmol) at 0° C. The reaction was stirred at ambient temperature overnight. The solvent was evaporated and the residue was partitioned between DCM (5 ml) and water, contains with citric acid (0.7 g, 3.64 mmol in water 1 ml). The extract was separated and dried over Na₂SO₄. The crude product 3 was used further without additional purification. Yield (155 mg, 85%). Colorless oil LCMS (C18 column 20×2 mm, 2.5 μm, pore size 100 Å, water-acetonitrile+0.1% TFA, gradient 5 to 87% for 3 min, retention time 1.43 min). MS (ESI) m/z 310.8 [MH]+. ¹H NMR (400 MHz, DMSO-d₆) δ 7.25 (d, J=7.0 Hz, 1H), 7.19 (d, J=8.5 Hz, 2H), 6.83 (d, J=8.5 Hz, 2H), 4.52 (m, 1H), 4.43 (br, 1H), 3.94-3.90 (m, 2H), 3.46-3.40 (m, 2H), 1.75-1.67 (m, 2H), 1.58-1.51 (m, 2H), 1.35 (s, 9H), 1.26 (d, J=7.0 Hz, 3H).

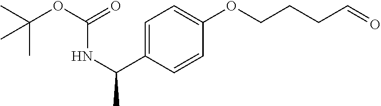 [SMILES: C[C@@H](NC(=O)OC(C)(C)C)c1ccc(OCCCC=O)cc1]

Compound 4. (R)-tert-butyl 1-(4-(4-oxobutoxy)phenyl)ethylcarbamate. A flask was charged with 10 mL of dichloromethane and oxalyl chloride (0.175 g, 1.38 mmol). The solution was stirred and cooled to −60° C. then dimethyl sulfoxide (0.108 g, 1.38 mmol) in 5 mL of dichloromethane was added dropwise at a rapid rate. After 5 min compound 3 (0.142 g, 0.46 mmol)) in 5 ml of dichloromethane was added dropwise maintaining the temperature at −50 to −60° C. After another 15 min, triethylamine (1.0 ml, 7.0 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 0.140 g (95%) of crude product 4 that used further without additional purification.

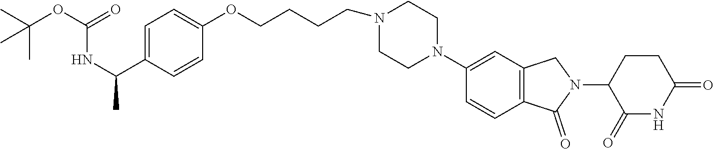 [SMILES: C[C@@H](NC(=O)OC(C)(C)C)c1ccc(OCCCCN2CCN(c3ccc4c(c3)CN(C3CCC(=O)NC3=O)C4=O)CC2)cc1]

Compound 5. tert-butyl (1R)-1-(4-(4-(4-(2-(2,6-dioxopiperidin-3-yl)-1-oxoisoindolin-5-yl)piperazin-1-yl)butoxy)phenyl)ethylcarbamate. To a suspension of 3-(1-oxo-5-(piperazin-1-yl)isoindolin-2-yl)piperidine-2,6-dione hydrochloride (A) (0.167 g, 0.46 mmol) and compound 4 (0.141 g, 0.46 mmol) in 10 mL of DCM was added DIPEA (0.15 mL, 0.92 mmol) and the mixture was stirred for an hour. Then sodium triacetoxyborohydride (0.390 g, 1.84 mmol) was added. The suspension was stirred at room temperature for 12 h and quenched with saturated NaHCO₃ aqueous solution. The product was extracted with dichloromethane (3 by 10 ml). After extraction, the combined organic layer was dried. Purification by silica gel (gradient ethyl acetate/tetrahydrofuran=100:1 to 1:100 as a developing solvent) afforded compound 5 (0.267 g, 90%). LCMS (C18 column 100×4.6 mm, 5.0 μm, pore size 100 Å, water-acetonitrile+0.1% TFA, gradient 5 to 87% for 10 min, retention time 4.94 min). MS (ESI) m/z 620.9 [MH]+.

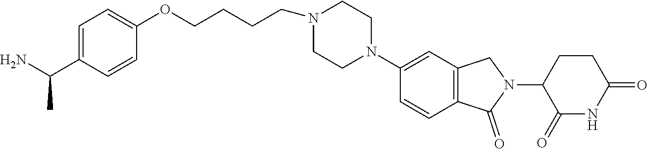 [SMILES: C[C@@H](N)c1ccc(OCCCCN2CCN(c3ccc4c(c3)CN(C3CCC(=O)NC3=O)C4=O)CC2)cc1]

Compound 6. 3-(5-(4-(4-(4-((R)-1-aminoethyl)phenoxy)butyl)piperazin-1-yl)-1-oxoisoindolin-2-yl)piperidine-2,6-dione hydrochloride. A solution of compound 5 (0.43 mmol, 1 eq) in dry DCM (50 mL) with 0.8 ml 3 M HCl in dioxane (5 eq) were stirred for 24 h at r.t. The mixture was evaporated to dryness to give the crude product 6 which was used in the next step without additional purification Yield 98%. LCMS (C18 column 100×4.6 mm, 5.0 μm, pore size 100 Å, water-acetonitrile+0.1% TFA, gradient 5 to 87% for 10 min, retention time 3.51 min). MS (ESI) m/z 520.7 [MH]+.

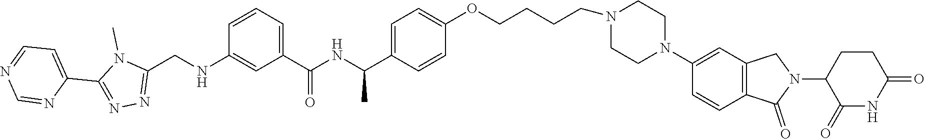 [SMILES: C[C@@H](NC(=O)c1cccc(NCc2nnc(-c3ccncn3)n2C)c1)c1ccc(OCCCCN2CCN(c3ccc4c(c3)CN(C3CCC(=O)NC3=O)C4=O)CC2)cc1]

D175. N-((1R)-1-(4-(4-(4-(2-(2,6-dioxopiperidin-3-yl)-1-oxoisoindolin-5-yl)piperazin-1-yl)butoxy)phenyl)ethyl)-3-((4-methyl-5-(pyrimidin-4-yl)-4H-1,2,4-triazol-3-yl)methylamino)benzamide hydrochloride. A mixture of compound 6 (150 mg, 0.254 mmol, 1 eq), compound B (79 mg, 0.254 mmol, 1 eq), TBTU (122 mg, 0.381 mmol, 2 eq), DIPEA (131 mg, 1.02 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile). Yield 49%. LCMS (C18 column 100×4.6 mm, 5.0 μm, pore size 100 Å, water-acetonitrile+0.1% TFA, gradient 5 to 87% for 10 min, retention time 4.49 min). MS (ESI) m/z 812.8 [MH]+. ¹H NMR (400 MHz, DMSO-d₆) δ 10.95 (s, 1H), 9.55 (br, 1H), 9.33 (s, 1H), 8.96 (d, J=5.3 Hz, 1H), 8.55 (d, J=8.2 Hz, 1H), 8.16 (dd, J=6.6, 1.4 Hz, 1H), 7.58 (d, J=4.4 Hz, 1H), 7.31 (d, J=8.7 Hz, 2H), 7.18-7.08 (m, 5H), 6.91-6.85 (m, 3H), 5.10-5.05 (m, 2H), 4.57 (s, 2H), 4.37-4.21 (m, 2H), 4.07-3.97 (m, 8H), 3.58 (br, 2H), 3.22 (br, 2H), 3.16-3.07 (m, 4H), 2.95-2.86 (m, 1H), 2.67-2.57 (m, 1H), 2.43-2.33 (m, 1H), 1.99-1.94 (m, 1H), 1.86-1.73 (m, 4H), 1.44 (d, J=7.0 Hz, 3H).

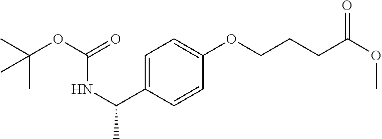 [SMILES: COC(=O)CCCOc1ccc([C@H](C)NC(=O)OC(C)(C)C)cc1]

Compound 2. (S)-methyl 4-(4-(1-(tert-butoxycarbonylamino)ethyl)phenoxy)butanoate. A mixture of compound 1 (200 mg, 0.45 mmol), methyl bromobutanoate (228 mg, 1.27 mmol), K₂CO₃ (354 g, 2.53 mmol), and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 284 mg (97%) of the desired product 2. ¹H NMR (400 MHz, DMSO-d₆) δ 7.24 (d, J=7.0 Hz, 1H), 7.17 (d, J=8.5 Hz, 2H), 6.84 (d, J=8.5 Hz, 2H), 4.54 (t, J=6.5 Hz, 1H), 3.94 (t, J=6.3 Hz, 2H), 3.60 (s, 3H), 2.45 (t, J=7.3 Hz, 2H), 1.97-1.92 (m, 2H), 1.35 (s, 9H), 1.25 (d, J=7.0 Hz, 3H).

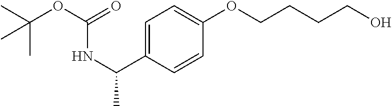 [SMILES: C[C@H](NC(=O)OC(C)(C)C)c1ccc(OCCCCO)cc1]

Compound 3. (S)-tert-butyl 1-(4-(4-hydroxybutoxy)phenyl)ethylcarbamate. To a mixture of compound 2 (284 mg, 0.842 mmol) in Et₂O (5 mL), LiBH₄ (27 mg, 1.26 mmol) was added, followed by methanol (40 mg, 1.26 mmol) at 0° C. The reaction was stirred at ambient temperature overnight. The solvent was evaporated and the residue was partitioned between DCM (5 ml) and water, contains with citric acid (0.7 g, 3.64 mmol in water 1 ml). The extract was separated and dried over Na₂SO₄. The crude product 3 was used further without additional purification. Yield (241 mg, 93%). Colorless oil LCMS (C18 column 20×2 mm, 2.5 μm, pore size 100 Å, water-acetonitrile+0.1% TFA, gradient 5 to 87% for 3 min, retention time 1.43 min). MS (ESI) m/z 310.8 [MH]+. ¹H NMR (400 MHz, DMSO-d₆) δ 7.25 (d, J=7.0 Hz, 1H), 7.19 (d, J=8.5 Hz, 2H), 6.83 (d, J=8.5 Hz, 2H), 4.52 (m, 1H), 4.43 (br, 1H), 3.94-3.90 (m, 2H), 3.46-3.40 (m, 2H), 1.75-1.67 (m, 2H), 1.58-1.51 (m, 2H), 1.35 (s, 9H), 1.26 (d, J=7.0 Hz, 3H).

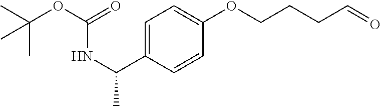 [SMILES: C[C@H](NC(=O)OC(C)(C)C)c1ccc(OCCCC=O)cc1]

Compound 4. (S)-tert-butyl 1-(4-(4-oxobutoxy)phenyl)ethylcarbamate. A flask was charged with 10 mL of dichloromethane and oxalyl chloride (0.297 g, 2.34 mmol). The solution was stirred and cooled to −60° C. then dimethyl sulfoxide (0.297 g, 2.34 mmol) in 5 mL of dichloromethane was added dropwise at a rapid rate. After 5 min compound 3 (0.241 g, 0.78 mmol) in 5 ml of dichloromethane was added dropwise maintaining the temperature at −50 to −60° C. After another 15 min, triethylamine (2.0 ml, 27 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 0.237 g (95%) of crude product 4 that used further without additional purification.

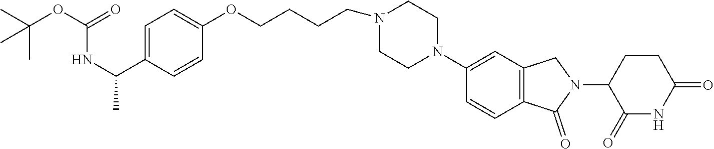 [SMILES: C[C@H](NC(=O)OC(C)(C)C)c1ccc(OCCCCN2CCN(c3ccc4c(c3)CN(C3CCC(=O)NC3=O)C4=O)CC2)cc1]

Compound 5. tert-butyl (1S)-1-(4-(4-(4-(2-(2,6-dioxopiperidin-3-yl)-1-oxoisoindolin-5-yl)piperazin-1-yl)butoxy)phenyl)ethylcarbamate. To a suspension of 3-(1-oxo-5-(piperazin-1-yl)isoindolin-2-yl)piperidine-2,6-dione hydrochloride (A) (0.284 g, 0.782 mmol) and compound 4 (0.240 g, 0.782 mmol) in 10 mL of DCM was added DIPEA (0.272 mL, 01.56 mmol) and the mixture was stirred for an hour. Then sodium triacetoxyborohydride (0.663 g, 3.128 mmol) was added. The suspension was stirred at room temperature for 12 h and quenched with saturated NaHCO₃ aqueous solution. The product was extracted with dichloromethane (3 by 10 ml). After extraction, the combined organic layer was dried. Purification by silica gel (gradient ethyl acetate/tetrahydrofuran=100:1 to 1:100 as a developing solvent) afforded compound 5 (0.403 g, 83%). LCMS (C18 column 100×4.6 mm, 5.0 μm, pore size 100 Å, water-acetonitrile+0.1% TFA, gradient 5 to 87% for 10 min, retention time 4.96 min). MS (ESI) m/z 620.9 [MH]+.

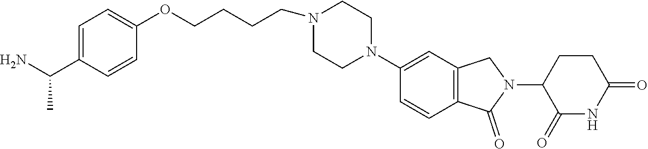 [SMILES: C[C@H](N)c1ccc(OCCCCN2CCN(c3ccc4c(c3)CN(C3CCC(=O)NC3=O)C4=O)CC2)cc1]

Compound 6. 3-(5-(4-(4-(4-((S)-1-aminoethyl)phenoxy)butyl)piperazin-1-yl)-1-oxoisoindolin-2-yl)piperidine-2,6-dione hydrochloride. A solution of compound 5 (0.646 mmol, 1 eq) in dry DCM (50 mL) with 1.3 ml 3 M HCl in dioxane (5 eq) were stirred for 24 h at r.t. The mixture was evaporated to dryness to give the crude product 6 which was used in the next step without additional purification. Yield 98%

LCMS (C18 column 100×4.6 mm, 5.0 μm, pore size 100 Å, water-acetonitrile+0.1% TFA, gradient 5 to 87% for 10 min, retention time 3.51 min). MS (ESI) m/z 520.7 [MH]+.

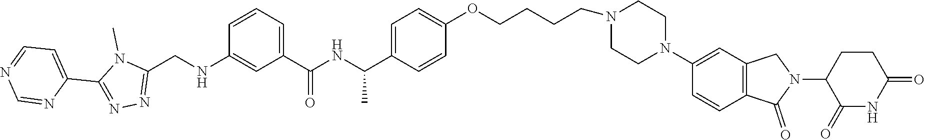 [SMILES: C[C@H](NC(=O)c1cccc(NCc2nnc(-c3ccncn3)n2C)c1)c1ccc(OCCCCN2CCN(c3ccc4c(c3)CN(C3CCC(=O)NC3=O)C4=O)CC2)cc1]

D117. N-((1S)-1-(4-(4-(4-(2-(2,6-dioxopiperidin-3-yl)-1-oxoisoindolin-5-yl)piperazin-1-yl)butoxy)phenyl)ethyl)-3-((4-methyl-5-(pyrimidin-4-yl)-4H-1,2,4-triazol-3-yl)methylamino)benzamide hydrochloride. A mixture of compound 6 (150 mg, 0.254 mmol, 1 eq), compound B (79 mg, 0.254 mmol, 1 eq), TBTU (122 mg, 0.381 mmol, 2 eq), DIPEA (131 mg, 1.02 mmol, 4 eq), and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile). Yield 65%. LCMS (C18 column 100×4.6 mm, 5.0 μm, pore size 100 Å, water-acetonitrile+0.1% TFA, gradient 5 to 87% for 10 min, retention time 4.49 min). MS (ESI) m/z 812.8 [MH]+. ¹H NMR (400 MHz, CDCl₃) δ 10.95 (s, 1H), 9.55 (br, 1H), 9.33 (s, 1H), 8.96 (d, J=5.3 Hz, 1H), 8.55 (d, J=8.2 Hz, 1H), 8.16 (dd, J=6.6, 1.4 Hz, 1H), 7.58 (d, J=4.4 Hz, 1H), 7.31 (d, J=8.7 Hz, 2H), 7.18-7.08 (m, 5H), 6.91-6.85 (m, 3H), 5.10-5.05 (m, 2H), 4.57 (s, 2H), 4.37-4.21 (m, 2H), 4.07-3.97 (m, 8H), 3.58 (br, 2H), 3.22 (br, 2H), 3.16-3.07 (m, 4H), 2.95-2.86 (m, 1H), 2.67-2.57 (m, 1H), 2.43-2.33 (m, 1H), 1.99-1.94 (m, 1H), 1.86-1.73 (m, 4H), 1.44 (d, J=7.0 Hz, 3H).

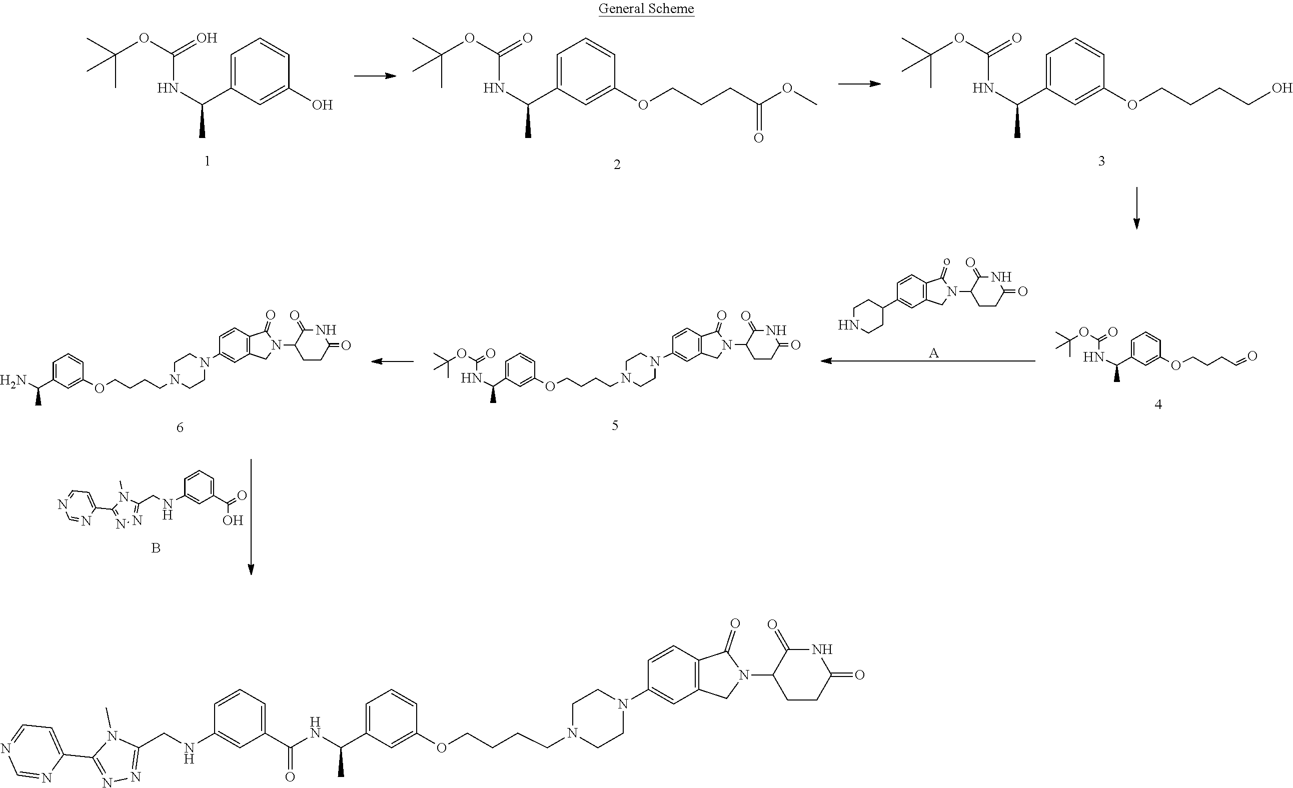

### Compounds, Procedures, and Analytical Data

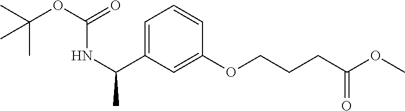 [SMILES: COC(=O)CCCOc1cccc([C@@H](C)NC(=O)OC(C)(C)C)c1]

Compound 2. (R)-methyl 4-(3-(1-(tert-butoxycarbonylamino)ethyl)phenoxy)butanoate. A mixture of compound 1 (350 mg, 1.45 mmol), methyl bromobutanoate (580 mg, 2.95 mmol), K₂CO₃ (812 g, 5.8 mmol) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 445 mg (91%) of the desired product 2. ¹H NMR (400 MHz, DMSO-d₆) δ 7.31 (d, J=7.0 Hz, 1H), 7.19 (t, J=8.5 Hz, 2H), 6.85-6.83 (m, 2H), 6.74 (dd, J=8.4, 1.5 Hz, 1H), 4.58 (t, J=6.5 Hz, 1H), 3.97 (t, J=6.3 Hz, 2H), 3.60 (s, 3H), 2.47 (t, J=7.3 Hz, 2H), 1.97-1.92 (m, 2H), 1.35 (s, 9H), 1.25 (d, J=7.0 Hz, 3).

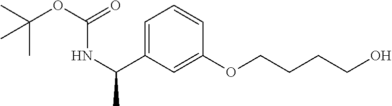 [SMILES: C[C@@H](NC(=O)OC(C)(C)C)c1cccc(OCCCCO)c1]

Compound 3. (R)-tert-butyl 1-(3-(4-hydroxybutoxy)phenyl)ethylcarbamate. Tao a mixture of compound 2 (445 mg, 1.32 mmol) in Et₂O (5 mL), LiBH₄ (43 mg, 1.98 mmol) was added, followed by methanol (64 mg, 1.98 mmol) at 0° C. The reaction was stirred at ambient temperature overnight. The solvent was evaporated and the residue was partitioned between DCM (5 ml) and water, contains with citric acid (0.7 g, 3.64 mmol in water 1 ml). The extract was separated and dried over Na₂SO₄. The crude product 3 was used further without additional purification. Yield (439 mg, 85%). Colorless oil LCMS (C18 column 20×2 mm, 2.5 μm, pore size 100 Å, water-acetonitrile+0.1% TFA, gradient 5 to 87% for 3 min, retention time 1.42 min). MS (ESI) m/z 310.6 [MH]+. H NMR (400 MHz, DMSO-d₆) δ 7.29 (d, J=7.0 Hz, 1H), 7.18 (t, J=8.5 Hz, 2H), 6.85-6.83 (m, 2H), 6.74 (dd, J=8.4, 1.5 Hz, 1H), 4.56 (m, 1H), 4.44 (t, J=5.1 Hz, 1H), 3.94-3.90 (t, J=6.5 Hz, 2H), 3.47-3.43 (m, 2H), 1.77-1.70 (m, 2H), 1.59-1.53 (m, 2H), 1.35 (s, 9H), 1.27 (d, J=7.0 Hz, 3H).

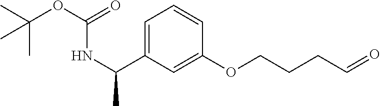 [SMILES: C[C@@H](NC(=O)OC(C)(C)C)c1cccc(OCCCC=O)c1]

Compound 4. (R)-tert-butyl 1-(3-(4-oxobutoxy)phenyl)ethylcarbamate. The flask was charged with 10 mL of dichloromethane and oxalyl chloride (0.252 g, 2.00 mmol). The solution was stirred and cooled at −50 to −60° C. as dimethyl sulfoxide (0.156 g, 2.00 mmol) in 5 ml of dichloromethane was added dropwise at a rapid rate. After 5 min compound 3 (0.410 g, 1.33 mmol) in 5 ml of dichloromethane was added dropwise maintaining the temperature at −50 to −60° C. After another 15 min, triethylamine (2.0 ml, 14.0 mmol) was added dropwise while keeping the temperature at or below −50° C. Stirring was continued for 5 min, after which time the mixture was allowed to warm to room temperature and 10 mL of water was added. The aqueous layer was separated and extracted with two 10 ml portions of dichloromethane. The organic layers were combined and dried over anhydrous magnesium sulfate. Rotary evaporation of the solvent gave 0.310 g (95%) of crude product 4 that was used further without additional purification.

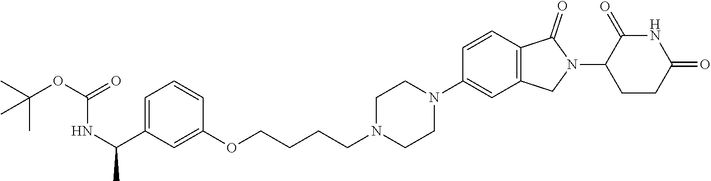 [SMILES: C[C@@H](NC(=O)OC(C)(C)C)c1cccc(OCCCCN2CCN(c3ccc4c(c3)CN(C3CCC(=O)NC3=O)C4=O)CC2)c1]

Compound 5. tert-butyl (1R)-1-(3-(4-(4-(2-(2,6-dioxopiperidin-3-yl)-1-oxoisoindolin-5-yl)piperazin-1-yl)butoxy)phenyl)ethylcarbamate. To a suspension of 3-(1-oxo-5-(piperazin-1-yl)isoindolin-2-yl)piperidine-2,6-dione hydrochloride (A) (0.441 g, 1.2 mmol) and compound 5 (0.310 g, 1.0 mmol) in 10 mL of DCM was added DIPEA (0.42 mL, 2.42 mmol) and the mixture was stirred for an hour. Then sodium triacetoxyborohydride (856 g, 4.04 mmol) was added. The suspension was stirred at room temperature for 12 h and quenched with saturated NaHCO₃ aqueous solution. The product was extracted with dichloromethane (3 by 10 ml). After extraction, the combined organic layer was dried. Purification by silica gel column chromatography (gradient ethyl acetate/tetrahydrofuran=100:1 to 1:100 as a developing solvent) afforded compound 5 (0.434 g, 69%). LCMS (C18 column 100×4.6 mm, 5.0 μm, pore size 100 Å, water-acetonitrile+0.1% TFA, gradient 5 to 87% for 10 min, retention time 5.10 min). MS (ESI) m/z 620.5 [MH]+

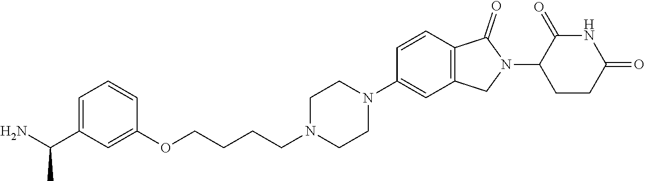 [SMILES: C[C@@H](N)c1cccc(OCCCCN2CCN(c3ccc4c(c3)CN(C3CCC(=O)NC3=O)C4=O)CC2)c1]

Compound 6. 3-(5-(4-(4-(3-((R)-1-aminoethyl)phenoxy)butyl)piperazin-1-yl)-1-oxoisoindolin-2-yl)piperidine-2,6-dione hydrochloride. A solution of compound 5 (189 mg, 0.305 mmol, 1 eq) in dry DCM (50 mL) with 0.6 ml 3 M HCl in dioxane (5 eq) were stirred for 24 h at r.t. The mixture was evaporated to dryness to give the crude product 6 which was used in the next step without additional purification. Yield 158 ms (98%)

LCMS (C18 column 100×4.6 mm, 5.0 μm, pore size 100 Å, water-acetonitrile+0.1% TFA, gradient 5 to 87% for 10 min, retention time 3.57 min). MS (ESI) m/z 520.7 [MH]+

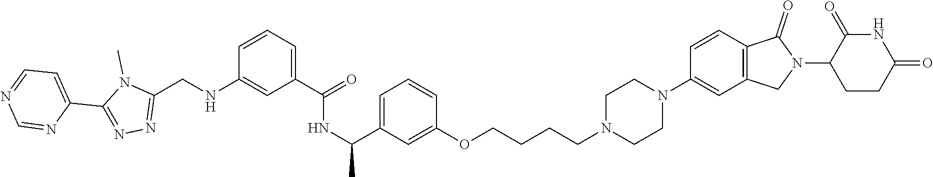 [SMILES: C[C@@H](NC(=O)c1cccc(NCc2nnc(-c3ccncn3)n2C)c1)c1cccc(OCCCCN2CCN(c3ccc4c(c3)CN(C3CCC(=O)NC3=O)C4=O)CC2)c1]

D129. N-((1R)-1-(3-(4-(4-(2-(2,6-dioxopiperidin-3-yl)-1-oxoisoindolin-5-yl)piperazin-1-yl)butoxy)phenyl)ethyl)-3-((4-methyl-5-(pyrimidin-4-yl)-4H-1,2,4-triazol-3-yl)methylamino)benzamide. A mixture of compound 6 (193 mg, 0.327 mmol, 1 eq), compound B (101 mg, 0.327 mmol, 1 eq), TBTU (157 mg, 0.490 mmol, 2 eq), DIPEA (167 mg, 1.29 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile). Yield 61%. LCMS (C18 column 100×4.6 mm, 5.0 μm, pore size 100 Å, water-acetonitrile+0.1% TFA, gradient 5 to 87% for 10 min, retention time 4.42 min). MS (ESI) m/z 812.8 [MH]+. ¹H NMR (400 MHz, CDCl₃) δ 10.93 (s, 1H), 9.53 (br, 1H), 9.33 (s, 1H), 8.97 (d, J=5.3 Hz, 1H), 8.55 (d, J=8.2 Hz, 1H), 8.17 (dd, J=6.6, 1.4 Hz, 1H), 7.58 (d, J=4.4 Hz, 1H), 7.26-7.12 (m, 6H), 6.98-6.89 (m, 3H), 6.79 (dd, J=7.8, 1.9 Hz, 1H), 5.13-5.03 (m, 2H), 4.58 (s, 2H), 4.37-4.21 (m, 2H), 4.01 (t, J=5.6 Hz, 2H), 3.85 (br, 5H), 3.60 (br, 2H), 3.22 (br, 2H), 3.16-3.07 (m, 4H), 2.95-2.86 (m, 1H), 2.67-2.57 (m, 1H), 2.43-2.33 (m, 1H), 1.99-1.94 (m, 1H), 1.88-1.75 (m, 4H), 1.44 (d, J=7.0 Hz, 3H).

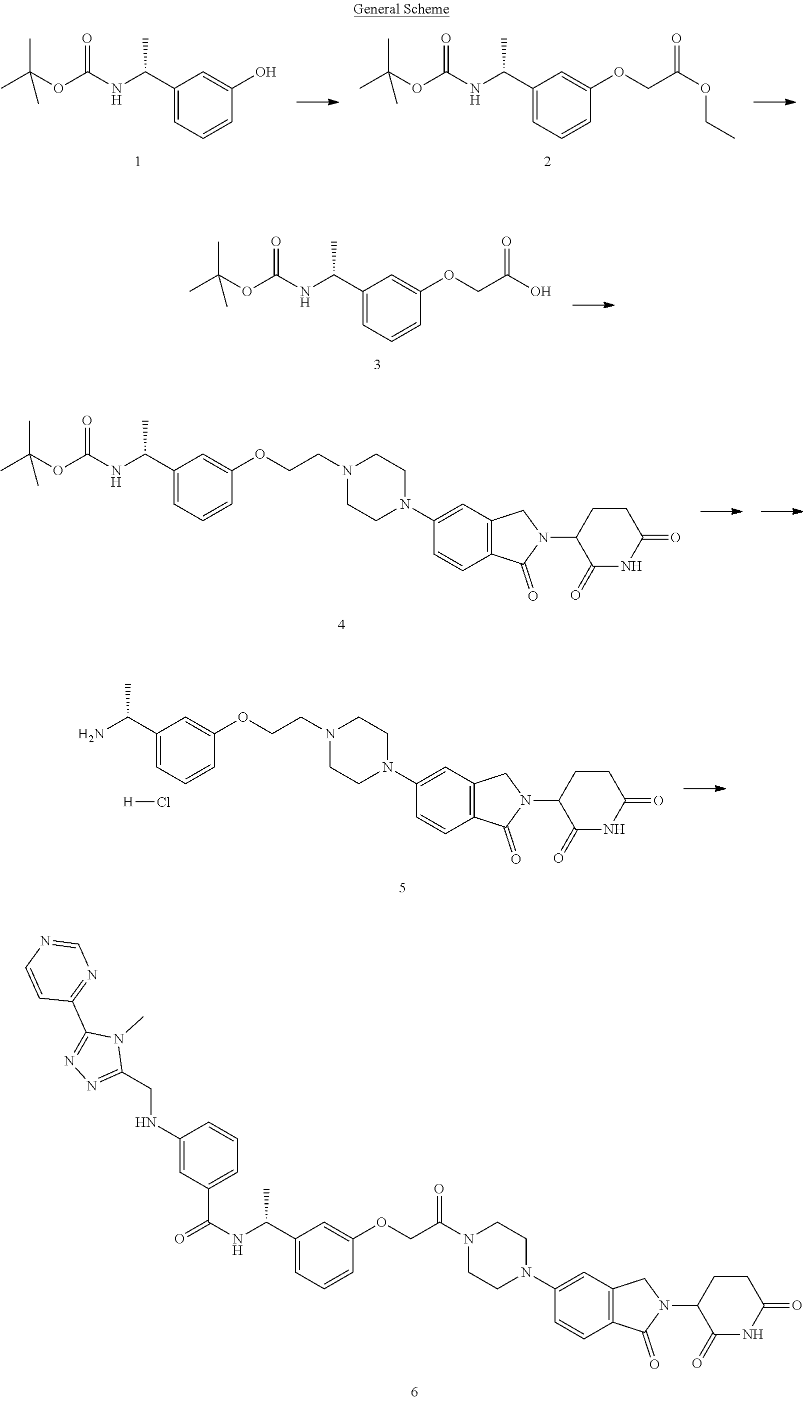

### Compounds, Procedures, and Analytical Data

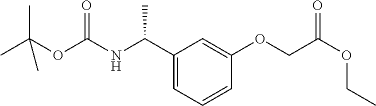 [SMILES: CCOC(=O)COc1cccc([C@@H](C)NC(=O)OC(C)(C)C)c1]

Compound 2. (R)-ethyl 2-(3-(1-(tert-butoxycarbonylamino)ethyl)phenoxy)acetate. To a solution of phenol 1 (0.42 g, 1.77 mmol) and ethyl chloroacetate (0.25 ml, 0.28 g, 2.33 mmol) in MeCN (5 ml), K2CO3 (0.8 g, 5.8 mmol) was added and the reaction mixture was refluxed with stirring for 48 hours. Then the solvent was evaporated and the residue was partitioned between methylene chloride (5 ml) and water (5 ml). The organic layer was separated; aqueous one was extracted with methylene chloride (5 ml). Combined organic layers were dried over MgSO4 and concentrated. The product 2 was chromatographed on silica, using chloroform and then a mixture chloroform-methanol (100 to 1) as an eluent. Yield 0.54 g, (94%). Colorless oil 94%. LCMS (C18 column 20×2 mm, 2.5 μm, pore size 100 Å, water-acetonitrile+0.1% TFA, gradient 5 to 87% for 3 min, retention time 1.61 min). MS (ESI) m/z 324.3 [MH]+. ¹H NMR (400 MHz, CDCl₃) δ 7.26 (t, J=7.6 Hz, 1H), 6.95 (d, J=7.6 Hz, 1H), 6.89 (s, 1H), 6.78 (dd, J=8.2, 2.4 Hz, 1H), 4.76 (s, 1H), 4.62 (s, 2H), 4.29 (q, J=7.2 Hz, 2H), 1.43 (s, 12H), 1.30 (t, J=7.2 Hz, 3H).

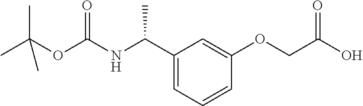 [SMILES: C[C@@H](NC(=O)OC(C)(C)C)c1cccc(OCC(=O)O)c1]

Compound 3. (R)-2-(3-(1-(tert-butoxycarbonylamino)ethyl)phenoxy)acetic acid. To a solution of ester 2 (0.65 g, 2.01 mmol) in methanol (5 ml), solid KOH (0.57 g, 10.16 mmol) was added and the mixture was stirred for 48 hours. The solvent was stripped off and the residue was partitioned between methylene chloride (5 ml) and a solution of KHSO4 (1.4 g, 10.28 mmol) in water (5 ml). The reaction mixture was stirred for an hour and the organic layer was separated; aqueous one was extracted with methylene chloride (5 ml). Combined organic layers were dried over MgSO4 and concentrated. Yield 0.42 g, (71%). Colorless oil 71%. LCMS (C18 column 20×2 mm, 2.5 μm, pore size 100 Å, water-acetonitrile+0.1% TFA, gradient 5 to 87% for 3 min, retention time 1.30 min). MS (ESI) m/z 296.5 [MH]+.

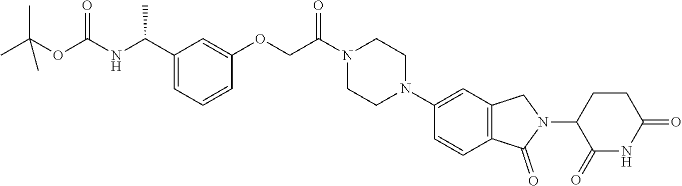 [SMILES: C[C@@H](NC(=O)OC(C)(C)C)c1cccc(OCC(=O)N2CCN(c3ccc4c(c3)CN(C3CCC(=O)NC3=O)C4=O)CC2)c1]

Compound 4. tert-butyl (1R)-1-(3-(2-(4-(2-(2,6-dioxopiperidin-3-yl)-1-oxoisoindolin-5-yl)piperazin-1-yl)-2-oxoethoxy)phenyl)ethylcarbamate. To a solution of acid 3 (0.1 g, 0.339 mmol) in dried pyridine (2 ml), lenalidomide (0.12 g, 0.329 mmol) was added, followed by TBTU (0.19 g, 0.501 mmol) and DIPEA (0.36 ml, 0.27 g, 2.08 mmol. The reaction mixture was stirred at ambient temperature and evaporated dryness. The residue was partitioned between methylene chloride (5 ml) and water (5 ml). The organic layer was separated; aqueous one was extracted with methylene chloride (5 ml). Combined organic layers were dried over MgSO4 and concentrated. The product 4 was purified by column chromatography using ethyl acetate and then a mixture ethyl acetate-THF (50 to 50) as an eluent. Yield 0.19 g (92%). Colorless oil 92%. LCMS (C18 column 20×2 mm, 2.5 μm, pore size 100 Å, water-acetonitrile+0.1% TFA, gradient 5 to 87% for 3 min, retention time 1.36 min). MS (ESI) m/z 606.3 [MH]+.

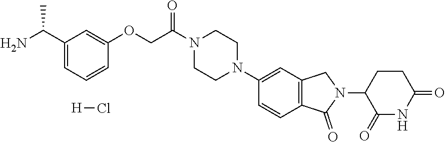

Compound 5. 3-(5-(4-(2-(3-((R)-1-aminoethyl)phenoxy)acetyl)piperazin-1-yl)-1-oxoisoindolin-2-yl)piperidine-2,6-dione hydrochloride. To an ice cold solution of ester 4 (0.19 g, 0.31 mmol) in methylene chloride (5 ml), dioxanic HCl solution (1.5 ml, 3M) was added and the mixture was stirred at room temperature for overnight. Then the solvent was evaporated, chloroform (5 ml) was added and evaporated again. The residue was crude 5 and used further without additional purification. Yield 0.17 g (100%). Colorless crystals 100%. LCMS (C18 column 20×2 mm, 2.5 μm, pore size 100 Å, water-acetonitrile+0.1% TFA, gradient 5 to 87% for 3 min, retention time 0.99 min). MS (ESI) m/z 506.4 [MH]+.

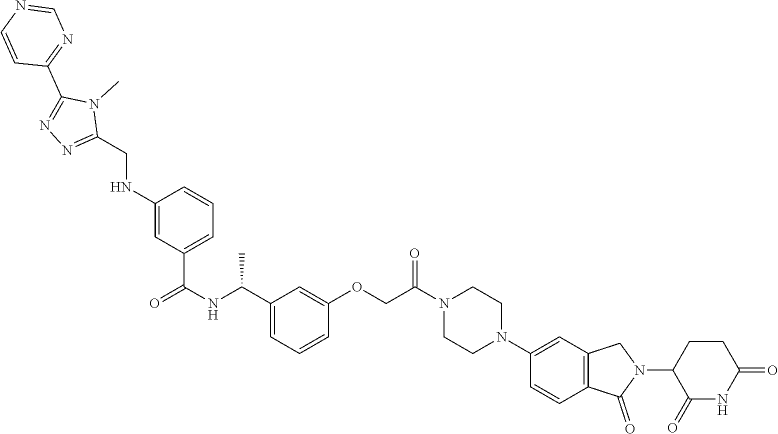 [SMILES: C[C@@H](NC(=O)c1cccc(NCc2nnc(-c3ccncn3)n2C)c1)c1cccc(OCC(=O)N2CCN(c3ccc4c(c3)CN(C3CCC(=O)NC3=O)C4=O)CC2)c1]

D176. Compound 6. N-((1R)-1-(3-(2-(4-(2-(2,6-dioxopiperidin-3-yl)-1-oxoisoindolin-5-yl)piperazin-1-yl)-2-oxoethoxy)phenyl)ethyl)-3-((4-methyl-5-(pyrimidin-4-yl)-4H-1,2,4-triazol-3-yl)methylamino)benzamide. To an ice cold suspension of amine hydrochloride 5 (0.17 g, 0.31 mmol) in methylene chloride (5 ml), 3-((4-methyl-5-(pyrimidin-4-yl)-4H-1,2,4-triazol-3-yl)methylamino)benzoic acid (0.11 g, 0.35 mmol) was added, followed by hydroxyl benzotriazole hydrate (0.08 g, 0.52 mmol) and EDCI (0.1 g, 0.52 mmol) and finally DIPEA (0.5 ml, 0.37 g, 2.9 mmol). The reaction mixture was stirred at ambient temperature overnight. Then water (5 ml) was added and organic layer was separated. Aqueous layer was extracted with methylene chloride (5 ml). Combined organic layers were dried over MgSO4 and evaporated. The product 6 was purified by HPLC chromatography. Yield of product 6 was 0.134 g (53%). LCMS (C18 column 20×2 mm, 2.5 μm, pore size 100 Å, water-acetonitrile+0.1% TFA, gradient 5 to 87% for 10 min, retention time 4.99 min). MS (ESI) m/z 798.5 [MH]+. ¹H NMR (400 MHz, DMSO-d₆) δ 10.94 (s, 1H), 9.33 (d, J=1.3 Hz, 1H), 8.96 (d, J=5.3 Hz, 1H), 8.58 (d, J=8.2 Hz, 1H), 8.17 (dd, J=5.3, 1.4 Hz, 1H), 7.54 (d, J=8.5 Hz, 1H), 7.24-7.14 (m, 3H), 7.11-7.06 (m, 3H), 6.99-6.95 (m, 2H), 6.89 (d, J=8.1 Hz, 1H), 6.79 (d, J=7.8 Hz, 1H), 6.45 (s, 1H), 5.19-4.97 (m, 2H), 4.85 (s, 2H), 4.57 (s, 2H), 4.25 (dt, J=32.8, 16.4 Hz, 2H), 4.06 (s, 3H), 3.61 (s, 5H), 2.98-2.79 (m, 1H), 2.67-2.50 (m, 3H), 2.36 (dd, J=17.5, 9.0 Hz, 1H), 2.05-1.83 (m, 1H), 1.44 (d, J=7.0 Hz, 3H).

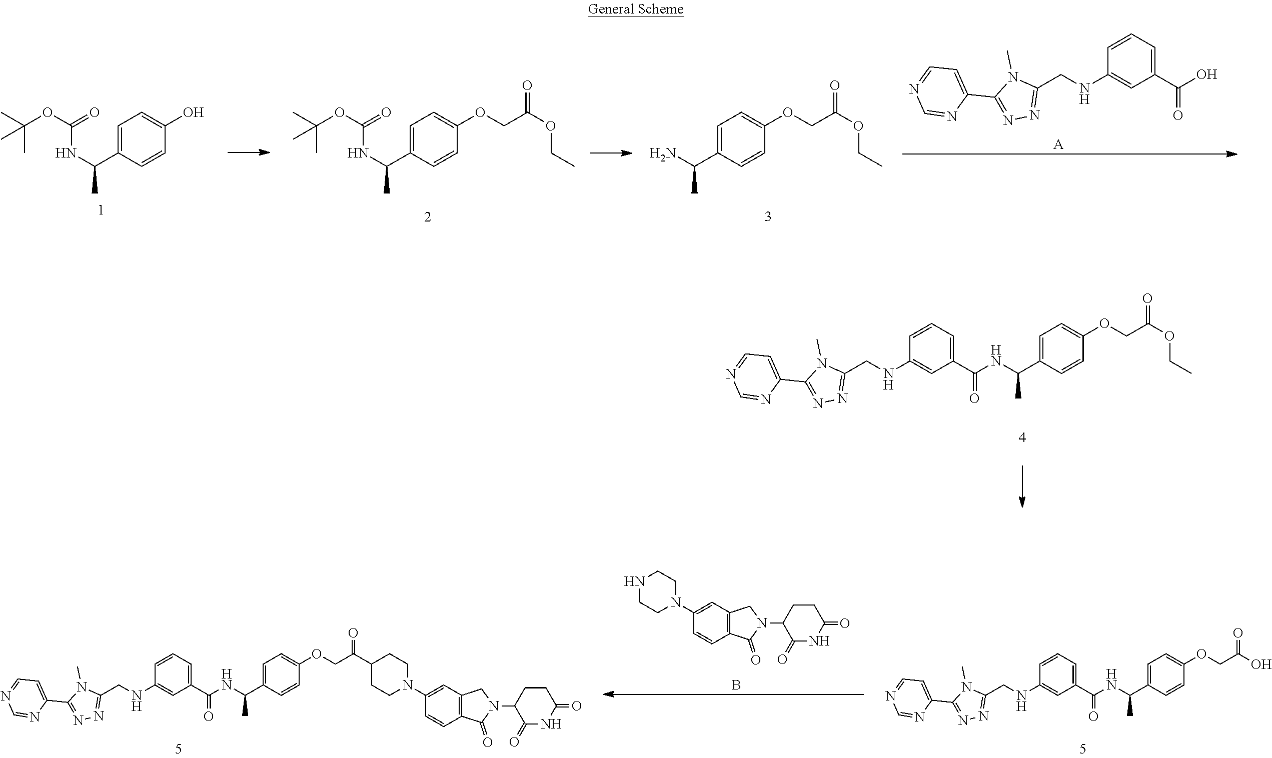

### Compounds, Procedures, and Analytical Data

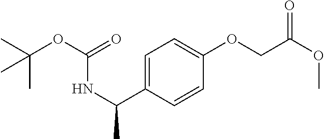 [SMILES: COC(=O)COc1ccc([C@@H](C)NC(=O)OC(C)(C)C)cc1]

Compound 2. (R)-methyl 2-(4-(1-(tert-butoxycarbonylamino)ethyl)phenoxy)acetate. A mixture of compound 1 (200 mg, 0.45 mmol), ethyl chloroacetate (154 mg, 1.27 mmol), K₂CO₃ (354 mg, 2.53 mmol) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford 270 mg (94%) of the desired product 2. ¹H NMR (400 MHz, DMSO-d₆) δ 7.26 (d, J=7.0 Hz, 1H), 7.18 (d, J=8.5 Hz, 2H), 6.85 (d, J=8.5 Hz, 2H), 4.72 (s, 2H), 4.33 (m, 1H), 4.18 (q, J=7.1 Hz, 2H), 1.35 (s, 9H), 1.27 (d, J=7.0 Hz, 3H), 1.20 (t, J=7.1 Hz, 3H).

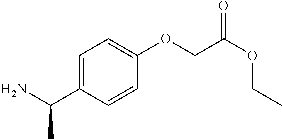 [SMILES: CCOC(=O)COc1ccc([C@@H](C)N)cc1]

Compound 3. (R)-ethyl 2-(4-(1-aminoethyl)phenoxy)acetate hydrochloride. A solution of compound 2 (0.619 mmol, 200 mg) in dry DCM (50 mL) with 1.3 ml 3 M HCl in dioxane (5 eq) were stirred for 24 h at r.t. The mixture was evaporated to dryness to give the crude product 3 which was used in the next step without additional purification. LCMS (C18 column 20×2 mm, 2.5 μm, pore size 100 Å, water-acetonitrile+0.1% TFA, gradient 5 to 87% for 3 min, retention time 0.92 min). MS (ESI) m/z 207.3[MH]+. ¹H NMR (400 MHz, DMSO-d₆) δ 8.35 (br, 3H), 7.41 (d, J=8.5 Hz, 2H), 6.98 (d, J=8.5 Hz, 2H), 4.79 (s, 2H), 4.33 (m, 1H), 4.17 (q, J=7.1 Hz, 2H), 1.47 (d, J=7.0 Hz, 3H), 1.21 (t, J=7.1 Hz, 3H).

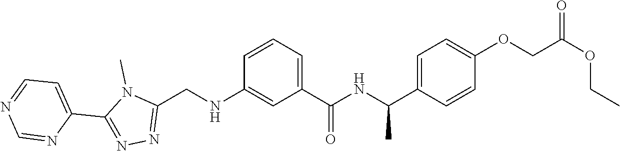 [SMILES: CCOC(=O)COc1ccc([C@@H](C)NC(=O)c2cccc(NCc3nnc(-c4ccncn4)n3C)c2)cc1]

Compound 4. (R)-ethyl 2-(4-(1-(3-((4-methyl-5-(pyrimidin-4-yl)-4H-1,2,4-triazol-3-yl)methylamino)benzamido)ethyl)phenoxy)acetate. A mixture of compound 3 (227 mg, 0.875 mmol, 1 eq), compound A (271 mg, 0.85 mmol, 1 eq), HOBt (173 mg, 1.14 mmol, 1.2 eq), EDCI (175 mg, 1.14 mmol, 1.2 eq), DIPEA (0.610 mL, 3.5 mmol, 4 eq) and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 4 (440 mg, 97%). LCMS (C18 column 100×4.6 mm, 5.0 μm, pore size 100 Å, water-acetonitrile+0.1% TFA, gradient 5 to 87% for 10 min, retention time 5.62 min). MS (ESI) m/z 516.3 [MH]+.

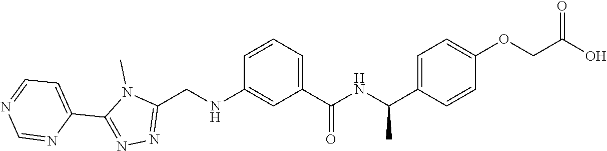 [SMILES: C[C@@H](NC(=O)c1cccc(NCc2nnc(-c3ccncn3)n2C)c1)c1ccc(OCC(=O)O)cc1]

Compound 5. (R)-2-(4-(1-(3-((4-methyl-5-(pyrimidin-4-yl)-4H-1,2,4-triazol-3-yl)methylamino)benzamido)ethyl)phenoxy)acetic acid. To a solution of compound 4 (440 mg, 0.854 mmol, 1 eq) in MeOH (20 mL) was added a solution KOH (144 mg, 2.56 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product 5 (277 mg, 67%) was used for the next step without additional purification. LCMS (C18 column 100×4.6 mm, 5.0 μm, pore size 100 Å, water-acetonitrile+0.1% TFA, gradient 5 to 87% for 10 min, retention time 4.97 min). MS (ESI) m/z 488.5 [MH]+

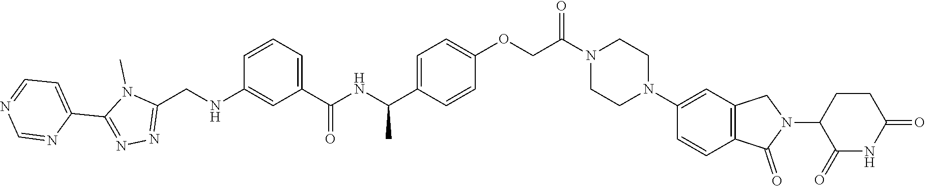 [SMILES: C[C@@H](NC(=O)c1cccc(NCc2nnc(-c3ccncn3)n2C)c1)c1ccc(OCC(=O)N2CCN(c3ccc4c(c3)CN(C3CCC(=O)NC3=O)C4=O)CC2)cc1]

D177. N-((1R)-1-(4-(2-(4-(2-(2,6-dioxopiperidin-3-yl)-1-oxoisoindolin-5-yl)piperazin-1-yl)-2-oxoethoxy)phenyl)ethyl)-3-((4-methyl-5-(pyrimidin-4-yl)-4H-1,2,4-triazol-3-yl)methylamino)benzamide. A mixture of compound 5 (100 mg, 0.205 mmol, 1 eq), compound B (75 mg, 0.205 mmol, 1 eq), TBTU (92 mg, 0.287 mmol, 2 eq), DIPEA (106 mg, 0.82 mmol, 4 eq) and anh. pyridine (3 mL) was stirred at ambient temperature for 12 h and concentrated under reduced pressure. The residue was treated with 3M HCl in dioxane, evaporated to dryness. The residue was purified by HPLC in acidic conditions (YMC-Pack ODS-AQ 250×20 mml, S-10 μm, 12 nm, gradient water-acetonitrile). Yield 37% LCMS (C18 column 100×4.6 mm, 5.0 μm, pore size 100 Å, water-acetonitrile+0.1% TFA, gradient 5 to 87% for 10 min, retention time 5.03 min). MS (ESI) m/z 798.4 [MH]+. ¹H NMR (400 MHz, DMSO-d₆) δ 10.94 (s, 1H), 9.33 (s, 1H), 8.97 (d, J=5.3 Hz, 1H), 8.54 (d, J=8.2 Hz, 1H), 8.17 (dd, J=6.6, 1.4 Hz, 1H), 7.55 (d, J=4.4 Hz, 1H), 7.30 (d, J=8.7 Hz, 2H), 7.20-7.07 (m, 5H), 6.90 (d, J=8.5 Hz, 3H), 6.45 (t, J=9.2 Hz, 1H), 5.11-5.03 (m, 2H), 4.84 (s, 2H), 4.56 (d, J=5.6 Hz, 2H), 4.36-4.16 (m, 5H), 4.07 (s, 3H), 3.62 (br, 4H), 3.38 (br, 1H), 2.94-2.85 (m, 1H), 2.66-2.56 (m, 1H), 2.43-2.33 (m, 1H), 2.00-1.94 (m, 1H), 1.45 (d, J=7.0 Hz, 3H).

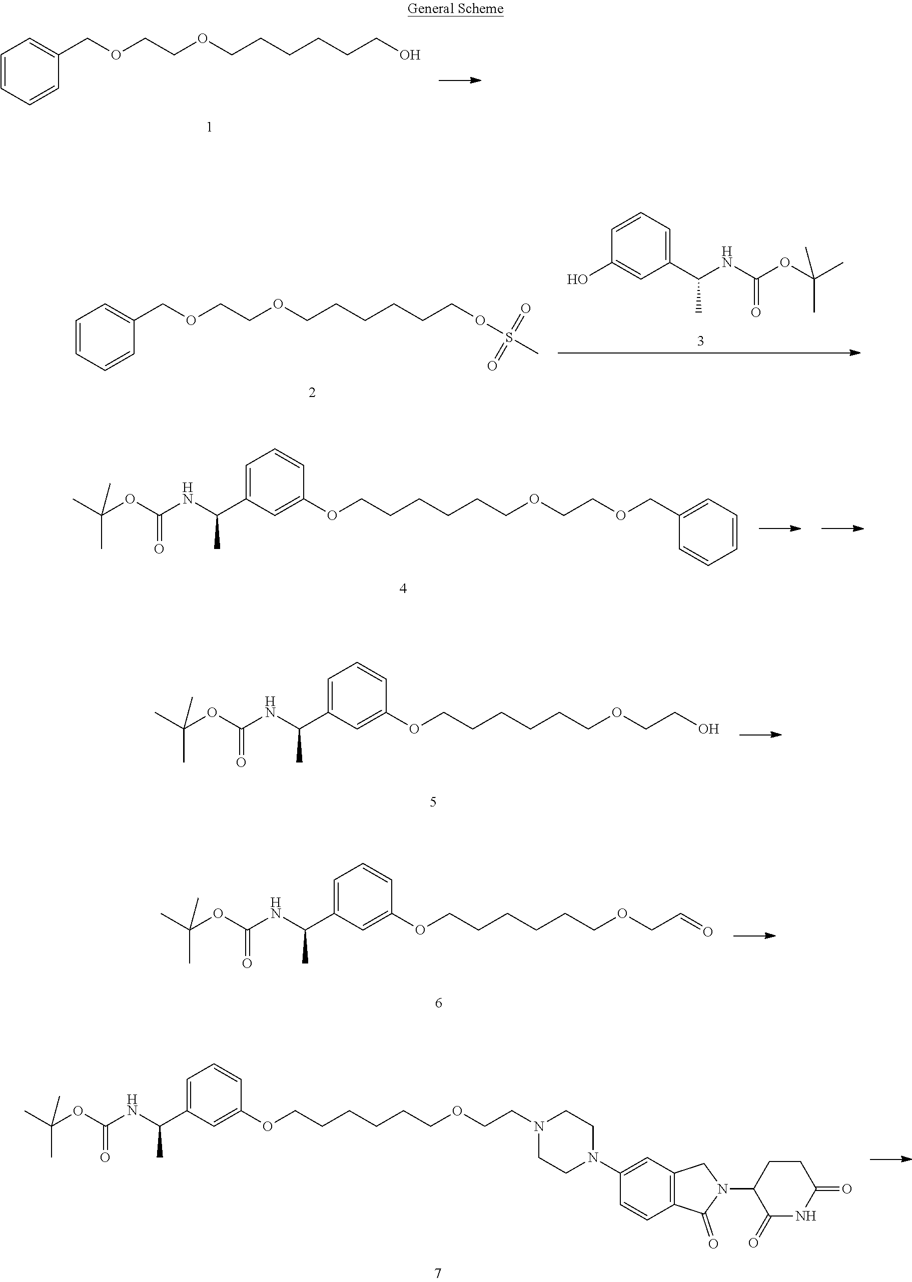

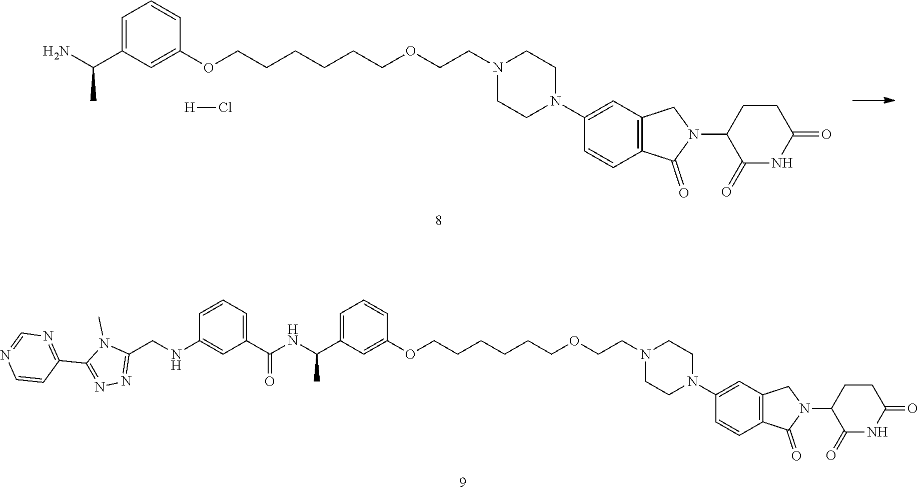 [SMILES: C[C@@H](N)c1cccc(OCCCCCCOCCN2CCN(c3ccc4c(c3)CN(C3CCC(=O)NC3=O)C4=O)CC2)c1 ; Cl ; C[C@@H](NC(=O)c1cccc(NCc2nnc(-c3ccncn3)n2C)c1)c1cccc(OCCCCCCOCCN2CCN(c3ccc4c(c3)CN(C3CCC(=O)NC3=O)C4=O)CC2)c1]

### Compounds, Procedures, and Analytical Data

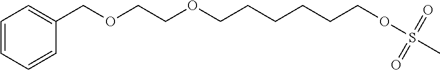 [SMILES: CS(=O)(=O)OCCCCCCOCCOCc1ccccc1]

Compound 2. 6-(2-(benzyloxy)ethoxy)hexyl methanesulfonate. To an ice cold solution of alcohol 1 (2.1 g, 8.32 mmol) in methylene chloride (10 ml), DIPEA (2.2 ml, 1.64 g, 12.71 mmol) was added, followed by mesyl chloride (0.8 ml, 1.15 g, 10.1 mmol). The reaction mixture was stirred at ambient temperature for 4 hours and quenched with water (10 ml). Organic phase was separated; aqueous was extracted with methylene chloride (10 ml). Combined organic phases were washed with water (10 ml) and dried over MgSO₄. The solvent was stripped off and the product 2 was used further without additional purification. Yield 2.75 g (100%).

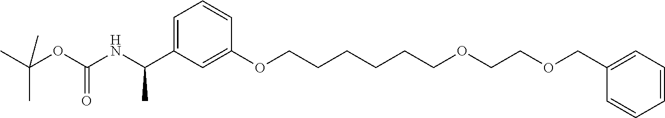 [SMILES: C[C@@H](NC(=O)OC(C)(C)C)c1cccc(OCCCCCCOCCOCc2ccccc2)c1]

Compound 4. (R)-tert-butyl 1-(3-(6-(2-(benzyloxy)ethoxy)hexyloxy)phenyl)ethylcarbamate. Compound 3 (1.8 g, 7.6 mmol, 1 eq), compound 2 (2.75 g, 8.32 mmol, 1.1 eq) and K₂C₃ (2.62 g, 19 mmol, 2.5 eq) in dry acetonitrile (50 mL) were stirred for 12 h at reflux. The solvent was evaporated to dryness. The residue was partitioned between methylene chloride (10 ml) and water (10 ml). Organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 3.5 g (89%). LCMS (C18 column 20×2 mm, 2.5 μm, pore size 100 Å, water-acetonitrile+0.1% TFA, gradient 5 to 87% for 3 min, retention time 2.21 min). MS (ESI) m/z 472.6 [MH]+.

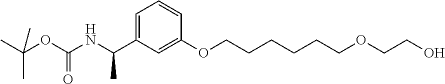 [SMILES: C[C@@H](NC(=O)OC(C)(C)C)c1cccc(OCCCCCCOCCO)c1]

Compound 5. (R)-tert-butyl 1-(3-(6-(2-hydroxyethoxy)hexyloxy)phenyl)ethylcarbamate. A stainless autoclave was charged with a solution of compound 4 (3.9 g, 8.27 mmol) in MeOH (50 ml) and 10% Pd on carbon (0.4 g) was added. The reaction was flushed with argon and hydrogenation was performed at 5 atm at ambient temperature overnight. Then the catalyst was filtered and washed with MeOH (10 ml). The filtrate was evaporated and used further without purification. Yield of compound 5 was 3.15 g (99%). LCMS (C18 column 20×2 mm, 2.5 μm, pore size 100 Å, water-acetonitrile+0.1% TFA, gradient 5 to 87% for 3 min, retention time 1.63 min). MS (ESI) m/z 382.6 [MH]+.

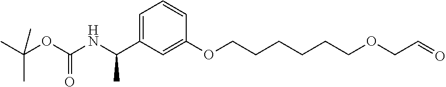 [SMILES: C[C@@H](NC(=O)OC(C)(C)C)c1cccc(OCCCCCCOCC=O)c1]

Compound 6. (R)-tert-butyl 1-(3-(6-(2-oxoethoxy)hexyloxy)phenyl)ethylcarbamate. A flask was charged with 10 mL of dichloromethane and oxalyl chloride (0.54 ml, 0.78 g, 6.2 mmol). The solution was stirred and cooled to −60° C. then dimethyl sulfoxide (0.65 ml 0.71 g, 9.1 mmol) in 5 mL of dichloromethane was added dropwise at a rapid rate. After 5 min compound 5 (1.45 g, 3.8 mmol) in 5 ml of dichloromethane was added dropwise maintaining the temperature at −50 to −60° C. After another 15 min, triethylamine (2.7 ml, 19.3 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 1.67 g (115%) of crude product 6 contains some amount of dimethyl sulfoxide and used further without additional purification. LCMS (C18 column 20×2 mm, 2.5 μm, pore size 100 Å, water-acetonitrile+0.1% TFA, gradient 5 to 87% for 3 min, retention time 1.49 min). MS (ESI) m/z 380.6 [MH]+.

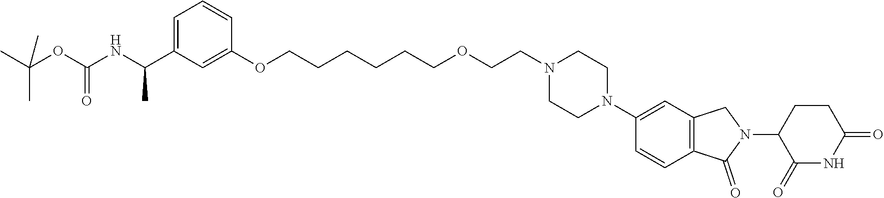 [SMILES: C[C@@H](NC(=O)OC(C)(C)C)c1cccc(OCCCCCCOCCN2CCN(c3ccc4c(c3)CN(C3CCC(=O)NC3=O)C4=O)CC2)c1]

Compound 7. tert-butyl (1R)-1-(3-(6-(2-(4-(2-(2,6-dioxopiperidin-3-yl)-1-oxoisoindolin-5-yl)piperazin-1-yl)ethoxy)hexyloxy)phenyl)ethylcarbamate. To a suspension of 3-(1-oxo-5-(piperazin-1-yl)isoindolin-2-yl)piperidine-2,6-dione hydrochloride (0.15 g, 0.41 mmol) in 10 mL of DCM was added DIPEA (0.20 mL, 1.15 mmol) and the mixture was stirred for an hour. Then compound 6 (0.2 g, 0.52 mmol) and sodium triacetoxyborohydride (0.35 g, 1.65 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 7 (0.10 g, 27%). Colorless oil. LCMS (C18 column 20×2 mm, 2.5 μm, pore size 100 Å, water-acetonitrile+0.1% TFA, gradient 5 to 87% for 3 min, retention time 1.36 min). MS (ESI) m/z 692.7 [MH]+

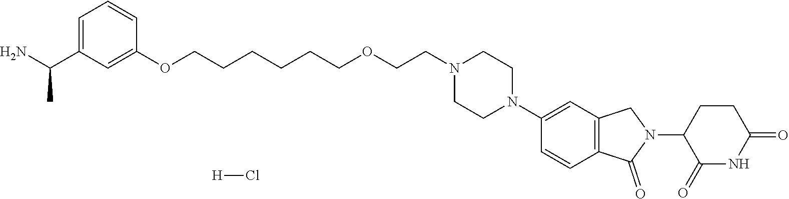 [SMILES: C[C@@H](N)c1cccc(OCCCCCCOCCN2CCN(c3ccc4c(c3)CN(C3CCC(=O)NC3=O)C4=O)CC2)c1 ; Cl]

Compound 8. 3-(5-(4-(2-(6-(3-((R)-1-aminoethyl)phenoxy)hexyloxy)ethyl)piperazin-1-yl)-1-oxoisoindolin-2-yl)piperidine-2,6-dione hydrochloride. To an ice cold solution of compound 7 (0.1 g, 0.14 mmol) in methylene chloride (5 ml), dioxanic HCl solution (0.5 ml, 3M) was added and the mixture was stirred at room temperature for overnight. Then the solvent was evaporated, chloroform (5 ml) was added and evaporated again. The residue was crude 8 and used further without additional purification. Yield 0.09 g (98%). LCMS (C18 column 20×2 mm, 2.5 μm, pore size 100 Å, water-acetonitrile+0.1% TFA, gradient 5 to 87% for 3 min, retention time 0.90 min). MS (ESI) m/z 592.8 [MH]+.

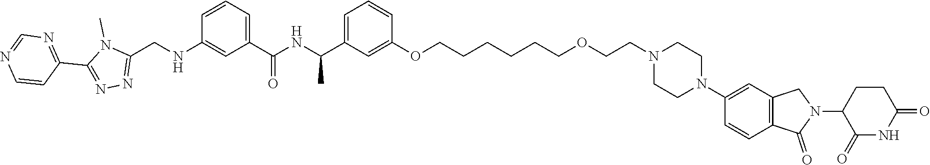 [SMILES: C[C@@H](NC(=O)c1cccc(NCc2nnc(-c3ccncn3)n2C)c1)c1cccc(OCCCCCCOCCN2CCN(c3ccc4c(c3)CN(C3CCC(=O)NC3=O)C4=O)CC2)c1]

Compound 9. D126. N-((1R)-1-(3-(6-(2-(4-(2-(2,6-dioxopiperidin-3-yl)-1-oxoisoindolin-5-yl)piperazin-1-yl)ethoxy)hexyloxy)phenyl)ethyl)-3-((4-methyl-5-(pyrimidin-4-yl)-4H-1,2,4-triazol-3-yl)methylamino)benzamide. To an ice cold suspension of amine hydrochloride 8 (0.09 g, 0.14 mmol) in methylene chloride (5 ml), 3-((4-methyl-5-(pyrimidin-4-yl)-4H-1,2,4-triazol-3-yl)methylamino)benzoic acid (0.05 g, 0.17 mmol) was added, followed by hydroxyl benzotriazole hydrate (0.03 g, 0.23 mmol) and EDCI (0.04 g, 0.23 mmol) and finally DIPEA (0.3 ml, 0.22 g, 1.73 mmol). The reaction mixture was stirred at ambient temperature overnight. Then water (5 ml) was added and organic layer was separated. Aqueous layer was extracted with methylene chloride (5 ml). Combined organic layers were dried over MgSO4 and evaporated. The residue was treated with dioxanic HCl (1 ml, 3M) and, after concentration, was purified by HPLC chromatography. Yield of product 9 was 0.014 g (10%). LCMS (C18 column 20×2 mm, 2.5 μm, pore size 100 Å, water-acetonitrile+0.1% TFA, gradient 5 to 87% for 10 min, retention time 4.91 min). MS (ESI) m/z 885.0 [MH]+. ¹H NMR (400 MHz, DMSO-d₆) δ 10.92 (s, 1H), 9.69 (s, 1H), 9.33 (s, 1H), 8.97 (d, J=5.3 Hz, 1H), 8.54 (d, J=8.2 Hz, 1H), 8.17 (d, J=5.2 Hz, 1H), 7.58 (d, J=8.3 Hz, 1H), 7.22-7.09 (m, 6H), 6.92 (t, J=11.1 Hz, 3H), 6.76 (d, J=7.9 Hz, 1H), 5.15-4.99 (m, 2H), 4.57 (s, 2H), 4.34 (d, J=16.7 Hz, 1H), 4.23 (d, J=17.0 Hz, 1H), 4.07 (s, 3H), 4.00-3.93 (m, 4H), 3.72 (s, 2H), 3.61-3.54 (m, 3H), 3.23-3.14 (m, 5H), 2.90 (t, J=12.8 Hz, 1H), 2.67-2.57 (m, 2H), 2.43-2.30 (m, 1H), 2.05-1.93 (m, 1H), 1.70 (t, J=6.8 Hz, 2H), 1.56 (t, J=6.8 Hz, 2H), 1.47-1.34 (m, 8H).

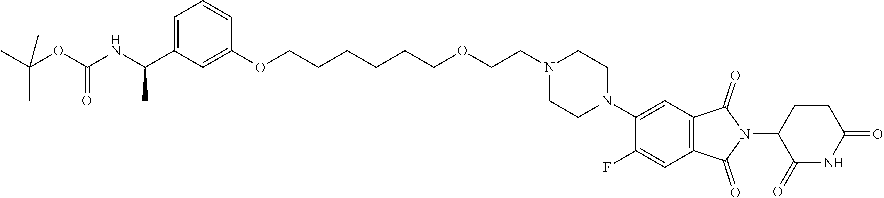 [SMILES: C[C@@H](NC(=O)OC(C)(C)C)c1cccc(OCCCCCCOCCN2CCN(c3cc4c(cc3F)C(=O)N(C3CCC(=O)NC3=O)C4=O)CC2)c1]

Compound 7. tert-butyl (1R)-1-(3-(6-(2-(4-(2-(2,6-dioxopiperidin-3-yl)-6-fluoro-1,3-dioxoisoindolin-5-yl)piperazin-1-yl)ethoxy)hexyloxy)phenyl)ethylcarbamate. To a suspension of 2-(2,6-dioxopiperidin-3-yl)-5-fluoro-6-(piperazin-1-yl)isoindoline-1,3-dione hydrochloride (0.2 g, 0.50 mmol) in 10 mL of DCM was added DIPEA (0.20 mL, 1.15 mmol) and the mixture was stirred for an hour. Then compound 6 (0.2 g, 0.52 mmol) and sodium triacetoxyborohydride (0.35 g, 1.65 mmol). The suspension was stirred at room temperature for 12 h and quenched with saturated NaHCO₃ aqueous solution. The product was extracted with dichloromethane (3 by 10 ml). After extraction, the combined organic layer was dried. Purification by silica gel (gradient ethyl acetate/tetrahydrofuran=100:1 to 1:100 as a developing solvent) afforded compound 7 (0.08 g, 21%). Colorless oil 21%. LCMS (C18 column 20×2 mm, 2.5 μm, pore size 100 Å, water-acetonitrile+0.1% TFA, gradient 5 to 87% for 3 min, retention time 1.45 min). MS (ESI) m/z 724.8 [MH]+

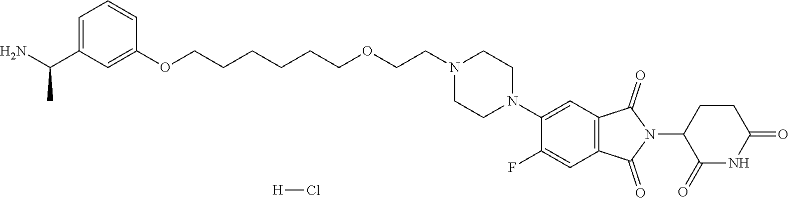 [SMILES: C[C@@H](N)c1cccc(OCCCCCCOCCN2CCN(c3cc4c(cc3F)C(=O)N(C3CCC(=O)NC3=O)C4=O)CC2)c1 ; Cl]

Compound 8. 5-(4-(2-(6-(3-((R)-1-aminoethyl)phenoxy)hexyloxy)ethyl)piperazin-1-yl)-2-(2,6-dioxopiperidin-3-yl)-6-fluoroisoindoline-1,3-dione hydrochloride. To an ice cold solution of compound 7 (0.08 g, 0.11 mmol) in methylene chloride (5 ml), dioxanic HCl solution (0.5 ml, 3M) was added and the mixture was stirred at room temperature for overnight. Then the solvent was evaporated, chloroform (5 ml) was added and evaporated again. The residue was crude 8 and used further without additional purification. Yield 0.08 g (100%). Colorless crystals 97%. LCMS (C18 column 20×2 mm, 2.5 μm, pore size 100 Å, water-acetonitrile+0.1% TFA, gradient 5 to 87% for 3 min, retention time 1.08 min). MS (ESI) m/z 624.5 [MH]+.

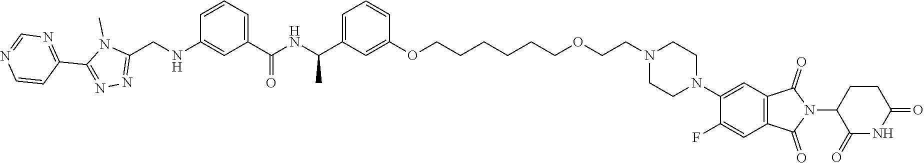 [SMILES: C[C@@H](NC(=O)c1cccc(NCc2nnc(-c3ccncn3)n2C)c1)c1cccc(OCCCCCCOCCN2CCN(c3cc4c(cc3F)C(=O)N(C3CCC(=O)NC3=O)C4=O)CC2)c1]

Compound 9. D134. N-((1R)-1-(3-(6-(2-(4-(2-(2,6-dioxopiperidin-3-yl)-6-fluoro-1,3-dioxoisoindolin-5-yl)piperazin-1-yl)ethoxy)hexyloxy)phenyl)ethyl)-3-((4-methyl-5-(pyrimidin-4-yl)-4H-1,2,4-triazol-3-yl)methylamino)benzamide. To an ice cold suspension of amine hydrochloride 8 (0.075 g, 0.11 mmol) in methylene chloride (5 ml), 3-((4-methyl-5-(pyrimidin-4-yl)-4H-1,2,4-triazol-3-yl)methylamino)benzoic acid (0.038 g, 0.12 mmol) was added, followed by hydroxyl benzotriazole hydrate (0.02 g, 0.15 mmol) and EDCI (0.03 g, 0.15 mmol) and finally DIPEA (0.2 ml, 0.15 g, 1.16 mmol). The reaction mixture was stirred at ambient temperature overnight. Then water (5 ml) was added and organic layer was separated. Aqueous layer was extracted with methylene chloride (5 ml). Combined organic layers were dried over MgSO4 and evaporated. The residue was treated with dioxanic HCl (1 ml, 3M) and, after concentration, was purified by HPLC chromatography. Yield of product 9 was 0.015 g (14%). LCMS (C18 column 20×2 mm, 2.5 μm, pore size 100 Å, water-acetonitrile+0.1% TFA, gradient 5 to 87% for 10 min, retention time 5.15 min). MS (ESI) m/z 917.0 [MH]+. ¹H NMR (400 MHz, DMSO-d₆) δ 11.10 (s, 1H), 9.75 (s, 1H), 9.33 (s, 1H), 8.97 (d, J=5.2 Hz, 1H), 8.55 (d, J=8.0 Hz, 1H), 8.17 (d, J=4.5 Hz, 1H), 7.80 (d, J=11.1 Hz, 1H), 7.59 (d, J=7.2 Hz, 1H), 7.26-7.13 (m, 3H), 7.10 (d, J=7.5 Hz, 1H), 6.94-6.89 (m, 2H), 6.76 (d, J=8.1 Hz, 1H), 5.17-5.00 (m, 2H), 4.57 (s, 2H), 4.07 (s, 3H), 3.94 (t, J=6.3 Hz, 2H), 3.80-3.72 (m, 5H), 3.61-3.55 (m, 4H), 3.31-3.25 (m, 4H), 2.94-2.84 (m, 1H), 2.67-2.55 (m, 2H), 2.35-2.29 (m, 1H), 2.07-2.03 (m, 1H), 1.73-1.66 (m, 2H), 1.63-1.52 (m, 2H), 1.46-1.33 (m, 8H).

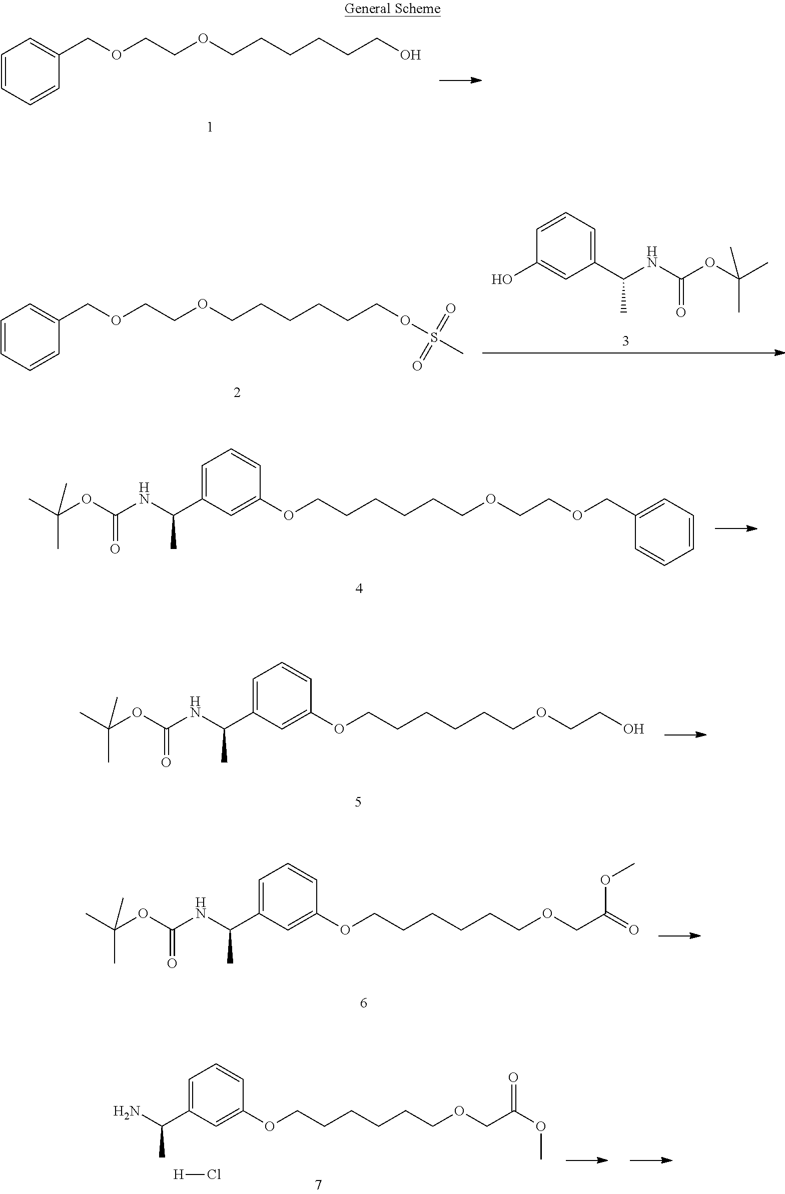

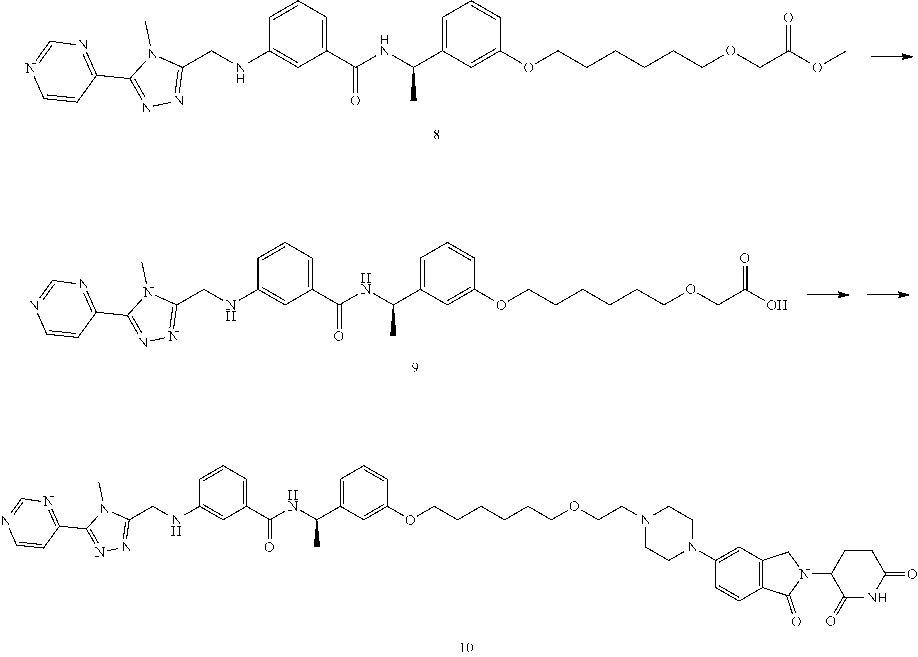 [SMILES: COC(=O)COCCCCCCOc1cccc([C@@H](C)NC(=O)c2cccc(NCc3nnc(-c4ccncn4)n3C)c2)c1 ; C[C@@H](NC(=O)c1cccc(NCc2nnc(-c3ccncn3)n2C)c1)c1cccc(OCCCCCCOCC(=O)O)c1 ; C[C@@H](NC(=O)c1cccc(NCc2nnc(-c3ccncn3)n2C)c1)c1cccc(OCCCCCCOCCN2CCN(c3ccc4c(c3)CN(C3CCC(=O)NC3=O)C4=O)CC2)c1]

### Compounds, Procedures, and Analytical Data

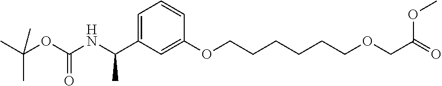 [SMILES: COC(=O)COCCCCCCOc1cccc([C@@H](C)NC(=O)OC(C)(C)C)c1]

Compound 6. (R)-methyl 2-(6-(3-(1-(tert-butoxycarbonylamino)ethyl)phenoxy)hexyloxy)acetate. To a solution of compound 5 (0.9 g, 2.36 mmol) in freshly distilled dimethylformamide (DMF, 20 mL) was slowly added pyridinium dichromate (PDC, 4.5 g, 11.96 mmol). The orange suspension was stirred at room temperature overnight. The next morning, H2O (20 mL) was added, and the solution was extracted with ether (3*50 mL). The combined ethereal layers were washed with H2O and saturated NaCl solution and dried (Na2SO4), and the volatiles were evaporated to yield 0.9 g of crude acid as a yellowish powder. This solid was dissolved in an ethereal solution of diazomethane at 0° C. and stirred for 15 min. The excess diazomethane was flushed out with nitrogen (under the hood), and the now clear solution was concentrated to furnish 0.70 g (75%) of compound 6 as a slightly yellow liquid. LCMS (C18 column 20×2 mm, 2.5 μm, pore size 100 Å, water-acetonitrile+0.1% TFA, gradient 5 to 87% for 3 min, retention time 1.86 min). MS (ESI) m/z 410.4 [MH]+.

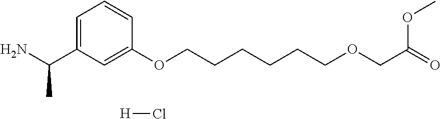 [SMILES: COC(=O)COCCCCCCOc1cccc([C@@H](C)N)c1 ; Cl]

Compound 7. (R)-methyl 2-(6-(3-(1-aminoethyl)phenoxy)hexyloxy)acetate hydrochloride. To an ice cold solution of compound 6 (0.7 g, 1.7 mmol) in methylene chloride (10 ml), dioxanic HCl solution (2.8 ml, 3M) was added and the mixture was stirred at room temperature for overnight. Then the solvent was evaporated, chloroform (5 ml) was added and evaporated again. The residue was crude 7 and used further without additional purification. Yield 0.60 g (100%). LCMS (C18 column 20×2 mm, 2.5 μm, pore size 100 Å, water-acetonitrile+0.1% TFA, gradient 5 to 87% for 3 min, retention time 1.14 min). MS (ESI) m/z 310.8 [MH]+.

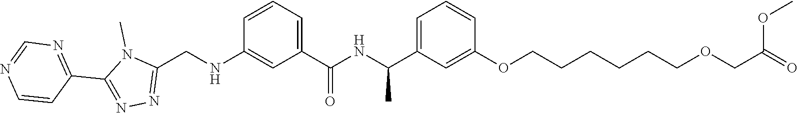 [SMILES: COC(=O)COCCCCCCOc1cccc([C@@H](C)NC(=O)c2cccc(NCc3nnc(-c4ccncn4)n3C)c2)c1]

Compound 8. (R)-methyl 2-(6-(3-(1-(3-((4-methyl-5-(pyrimidin-4-yl)-4H-1,2,4-triazol-3-yl)methylamino)benzamido)ethyl)phenoxy)hexyloxy)acetate. To an ice cold suspension of amine hydrochloride 7 (0.6 g, 0.14 mmol) in methylene chloride (10 ml), 3-((4-methyl-5-(pyrimidin-4-yl)-4H-1,2,4-triazol-3-yl)methylamino)benzoic acid (0.5 g, 1.7 mmol) was added, followed by hydroxyl benzotriazole hydrate (0.4 g, 2.61 mmol) and EDCI (0.5 g, 2.61 mmol) and finally DIPEA (2.0 ml, 1.5 g, 11.58 mmol). The reaction mixture was stirred at ambient temperature overnight. Then water (10 ml) was added and organic layer was separated. Aqueous layer was extracted with methylene chloride (10 ml). Combined organic layers were dried over MgSO4 and evaporated. The residue was purified by chromatography on silica, using chloroform-methanol (20 to 1) mixture as an eluent. Yield of product 8 was 0.49 g (46%). LCMS (C18 column 20×2 mm, 2.5 μm, pore size 100 Å, water-acetonitrile+0.1% TFA, gradient 5 to 87% for 3 min, retention time 1.48 min). MS (ESI) m/z 602.3 [MH]+. ¹H NMR (400 MHz, CDCl₃) δ 9.27 (s, 1H), 8.87 (d, J=5.2 Hz, 1H), 8.27 (d, J=5.1 Hz, 1H), 7.26-7.22 (m, 2H), 7.15-7.07 (m, 1H), 6.98 (t, J=10.0 Hz, 1H), 6.93-6.90 (m, 2H), 6.80 (t, J=6.8 Hz, 1H), 6.47-6.45 (m, 1H), 5.32-5.28 (m, 1H), 4.57 (s, 3H), 4.15 (s, 3H), 4.08 (s, 2H), 3.95 (t, J=6.4 Hz, 1H), 3.81 (s, 2H), 3.75 (s, 2H), 3.53 (dt, J=18.2, 8.9 Hz, 2H), 1.82-1.74 (m, 2H), 1.70-1.56 (m, 6H), 1.53-1.37 (m, 4H).

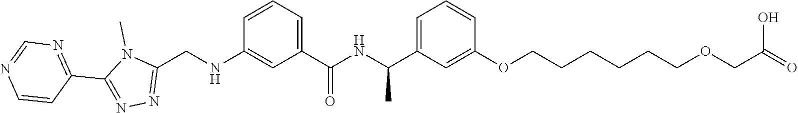 [SMILES: C[C@@H](NC(=O)c1cccc(NCc2nnc(-c3ccncn3)n2C)c1)c1cccc(OCCCCCCOCC(=O)O)c1]

Compound 9. (R)-2-(6-(3-(1-(3-((4-methyl-5-(pyrimidin-4-yl)-4H-1,2,4-triazol-3-yl)methylamino)benzamido)ethyl)phenoxy)hexyloxy)acetic acid. To a solution of ester 8 (0.49 g, 0.81 mmol) in methanol (5 ml), solid KOH (0.24 g, 4.3 mmol) was added and the mixture was stirred for 48 hours. The solvent was stripped off and the residue was partitioned between methylene chloride (5 ml) and a solution of KHSO4 (0.59 g, 4.3 mmol) in water (5 ml). The reaction mixture was stirred for an hour and the organic layer was separated; aqueous one was extracted with methylene chloride (5 ml). Combined organic layers were dried over MgSO4 and concentrated. Yield 0.36 g, (75%). Colorless oil 75%. LCMS (C18 column 20×2 mm, 2.5 μm, pore size 100 Å, water-acetonitrile+0.1% TFA, gradient 5 to 87% for 3 min, retention time 1.34 min). MS (ESI) m/z 588.3 [MH]+.

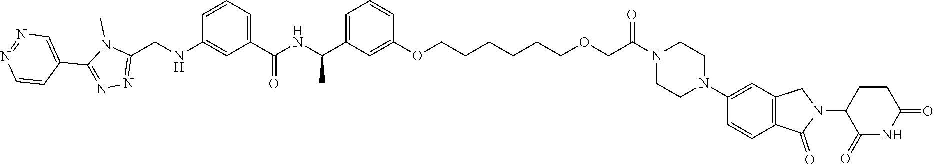 [SMILES: C[C@@H](NC(=O)c1cccc(NCc2nnc(-c3ccnnc3)n2C)c1)c1cccc(OCCCCCCOCC(=O)N2CCN(c3ccc4c(c3)CN(C3CCC(=O)NC3=O)C4=O)CC2)c1]

Compound 10. D132. N-((1R)-1-(3-(6-(2-(4-(2-(2,6-dioxopiperidin-3-yl)-1-oxoisoindolin-5-yl)piperazin-1-yl)-2-oxoethoxy)hexyloxy)phenyl)ethyl)-3-((4-methyl-5-(pyrimidin-4-yl)-4H-1,2,4-triazol-3-yl)methylamino)benzamide. To a solution of acid 9 (0.1 g, 0.17 mmol) in dried pyridine (2 ml), 2-(2,6-dioxopiperidin-3-yl)-5-fluoro-6-(piperazin-1-yl)isoindoline-1,3-dione hydrochloride (0.06 g, 0.17 mmol) was added, followed by TBTU (0.09 g, 0.253 mmol) and DIPEA (0.2 ml, 1.15 mmol). The reaction mixture was stirred at ambient temperature and evaporated dryness. The residue was partitioned between methylene chloride (5 ml) and water (5 ml). The organic layer was separated; aqueous one was extracted with methylene chloride (5 ml). Combined organic layers were dried over MgSO4 and concentrated. The product 10 was purified by HPLC chromatography. Yield 0.042 g (27%). LCMS (C18 column 20×2 mm, 2.5 μm, pore size 100 Å, water-acetonitrile+0.1% TFA, gradient 5 to 87% for 10 min, retention time 5.67 min). MS (ESI) m/z 898.6 [MH]+. ¹H NMR (400 MHz, DMSO-d₆) δ 10.91 (s, 1H), 9.32 (d, J=1.3 Hz, 1H), 8.96 (d, J=5.3 Hz, 1H), 8.53 (d, J=8.2 Hz, 1H), 8.17 (dd, J=5.3, 1.4 Hz, 1H), 7.53 (d, J=8.7 Hz, 1H), 7.21-7.16 (m, 3H), 7.11-7.05 (m, 2H), 6.93-6.89 (m, 2H), 6.75 (d, J=7.1 Hz, 1H), 6.42 (d, J=5.6 Hz, 1H), 5.15-4.97 (m, 2H), 4.57 (d, J=5.5 Hz, 2H), 4.32 (d, J=16.9 Hz, 1H), 4.21 (d, J=16.8 Hz, 1H), 4.14 (s, 2H), 4.07 (s, 3H), 3.93 (t, J=6.4 Hz, 2H), 3.61-3.54 (m, 4H), 3.43 (t, J=6.4 Hz, 2H), 3.35-3.25 (m, 5H), 2.88 (dd, J=21.9, 9.0 Hz, 1H), 2.67-2.50 (m, 2H), 2.42-2.28 (m, 1H), 2.05-1.91 (m, 1H), 1.76-1.62 (m, 2H), 1.61-1.48 (m, 2H), 1.40 (dd, J=24.7, 9.3 Hz, 7H).

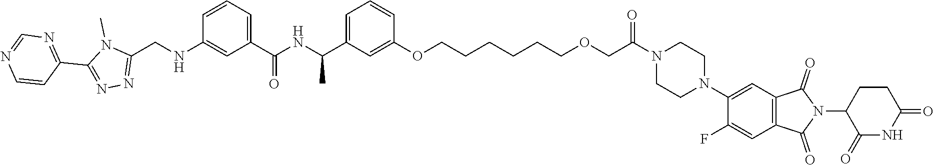 [SMILES: C[C@@H](NC(=O)c1cccc(NCc2nnc(-c3ccncn3)n2C)c1)c1cccc(OCCCCCCOCC(=O)N2CCN(c3cc4c(cc3F)C(=O)N(C3CCC(=O)NC3=O)C4=O)CC2)c1]

Compound 10. D133. N-((1R)-1-(3-(6-(2-(4-(2-(2,6-dioxopiperidin-3-yl)-6-fluoro-1,3-dioxoisoindolin-5-yl)piperazin-1-yl)-2-oxoethoxy)hexyloxy)phenyl)ethyl)-3-((4-methyl-5-(pyrimidin-4-yl)-4H-1,2,4-triazol-3-yl)methylamino)benzamide. To a solution of acid 9 (0.1 g, 0.17 mmol) in dried pyridine (2 ml), 2-(2,6-dioxopiperidin-3-yl)-5-fluoro-6-(piperazin-1-yl)isoindoline-1,3-dione hydrochloride (0.06 g, 0.17 mmol) was added, followed by TBTU (0.09 g, 0.253 mmol) and DIPEA (0.2 ml, 1.15 mmol). The reaction mixture was stirred at ambient temperature and evaporated dryness. The residue was partitioned between methylene chloride (5 ml) and water (5 ml). The organic layer was separated; aqueous one was extracted with methylene chloride (5 ml). Combined organic layers were dried over MgSO4 and concentrated. The product 10 was purified by HPLC chromatography. Yield 0.048 g (30%). LCMS (C18 column 20×2 mm, 2.5 μm, pore size 100 Å, water-acetonitrile+0.1% TFA, gradient 5 to 87% for 10 min, retention time 6.26 min). MS (ESI) m/z 930.7 [MH]+. ¹H NMR (400 MHz, DMSO-d₆) δ 11.08 (s, 1H), 9.32 (d, J=1.3 Hz, 1H), 8.96 (d, J=5.3 Hz, 1H), 8.53 (d, J=8.2 Hz, 1H), 8.17 (dd, J=5.2, 1.4 Hz, 1H), 7.74 (d, J=11.3 Hz, 1H), 7.47 (d, J=7.4 Hz, 1H), 7.21-7.16 (m, 2H), 7.10 (d, J=7.7 Hz, 1H), 6.98-6.84 (m, 2H), 6.76 (d, J=6.8 Hz, 1H), 6.43 (t, J=5.7 Hz, 1H), 5.10 (dd, J=12.8, 5.4 Hz, 2H), 4.57 (d, J=5.7 Hz, 2H), 4.14 (s, 2H), 4.08 (s, 3H), 3.93 (t, J=6.4 Hz, 2H), 3.60 (s, 4H), 3.43 (t, J=6.5 Hz, 2H), 3.29-3.27 (m, 5H), 2.97-2.80 (m, 1H), 2.70-2.55 (m, 2H), 2.13-1.96 (m, 1H), 1.77-1.64 (m, 2H), 1.61-1.49 (m, 2H), 1.44-1.39 (m, 8H).

### Example 23: Preparation of Compound D178, D179, D124, D125, D127, D130, D154, and D163

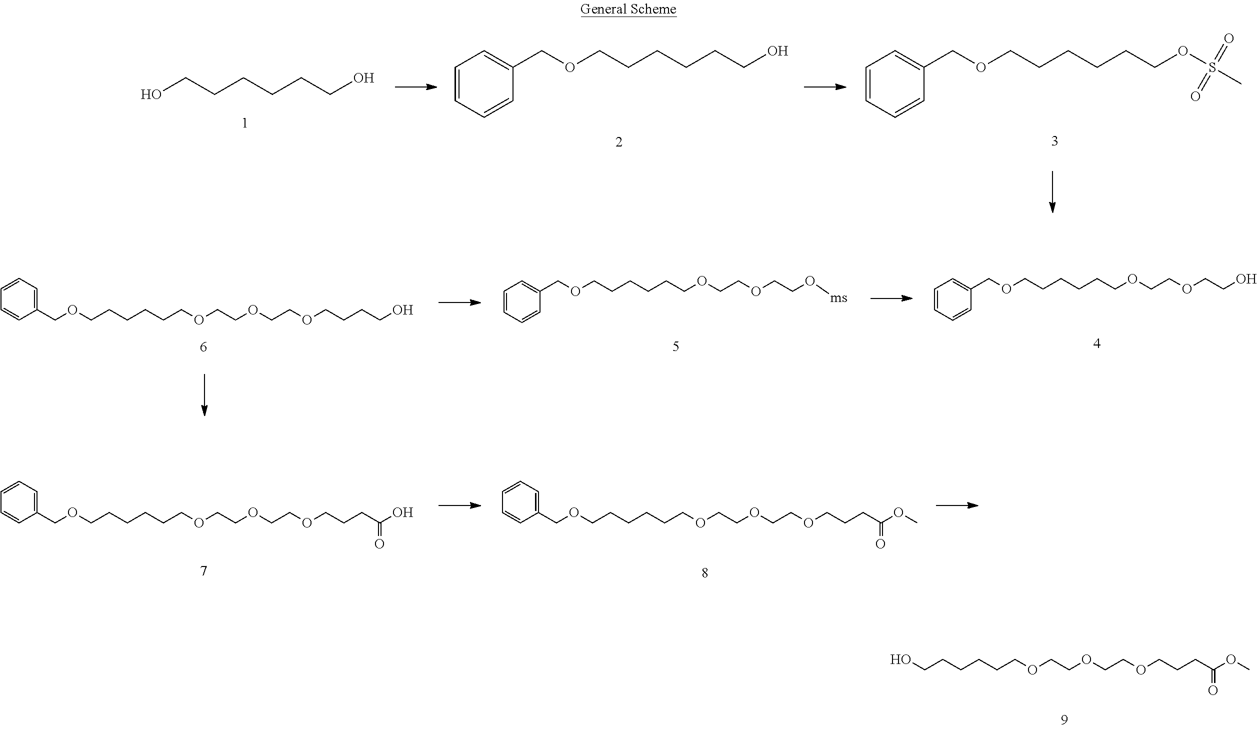

### Compounds, Procedures, and Analytical Data

Compound 2 A mixture of compounds 1 (65.8 g, 631 mmol, 4 eq), benzyl chloride (20.0 g, 158 mmol, 1 eq.), TBAB (0.2 g) and 50% aqua solution of NaOH (25.3 g, 631 mmol, 1 eq) was stirred at 100° C. for 12 h before been poured in water (200 ml). The product was extracted with Et₂O (3×150 ml), the organic layers were dried over Na₂SO₄, the solvent was removed under reduced pressure. The residue was purified by column chromatography on silica gel, using hexane/EtOAc 1:1 as eluent, to provide the product 2. Yield 85%. ¹H NMR (400 MHz, DMSO-d₆) δ 7.36-7.27 (m, 5H), 4.43 (s, 2H), 4.33 (t, J=4.3 Hz, 1H), 3.42-3.37 (m, 4H), 1.56-1.49 (m, 2H), 1.44-1.37 (m, 2H), 1.35-1.25 (m, 4H).

Compound 3. A solution of compound 2 (15.8 g, 75.85 mmol) in CH₂Cl₂ was mixed with 19.8 mL of DIPEA (113.77 mmol) and cooled to 0° C. 10.42 g of MsCl was added dropwise. The mixture was stirred at room temperature for 2 hours, washed with water, dried over Na₂SO₄ and evaporated. The product was used without purification. Yield 21.9 g (98%). ¹H NMR (400 MHz, DMSO-d₆) δ 7.36-7.27 (m, 5H), 4.44 (s, 2H), 4.19 (t, J=6.2 Hz, 2H), 3.42 (t, J=6.4 Hz, 2H), 3.15 (s, 3H), 1.69-1.62 (m, 2H), 1.56-1.51 (m, 2H), 1.41-1.30 (m, 4H).

Compound 4. An oven-dried 500 mL flask with stirrer was charged with sodium hydride (60% dispersion in mineral oil, 7.5 g, 187 mmol). The flask was sealed with a septum, evacuated and back-filled with argon (3 times). Anhydrous DMF (100 mL) followed by diethylene glycol (24.1 g, 227.5 mmol) were added and the reaction mixture stirred at 25° C. for 2 hr. Solution of 5-benzyloxyhexthyl 4-methanesulfonate 3 (21.7 g, 75.8 mmol) in anhydrous DMF (200 mL) was added to the mixture. The solution was warmed to 50° C. and stirred for 18 hr before been cooled to r.t. and concentrated in vacuo. The residue was partitioned between H₂O (400 mL) and Et₂O (500 mL). The organic layer was dried over Na₂SO₄ and concentrated in vacuo. The crude yellow oil was then purified by flash chromatography (200 g SiO₂, EtOAc and hexane (50/50)) providing compound 4 (8.5 g, 38% yield) as viscous oil. ¹H NMR (400 MHz, DMSO-d₆) δ 7.36-7.27 (m, 5H), 4.55 (t, J=5.5 Hz, 1H), 4.44 (s, 2H), 3.51-3.45 (m, 6H), 3.42-3.39 (m, 4H), 3.36 (t, J=6.6 Hz, 2H), 1.55-1.46 (m, 4H), 1.33-1.27 (m, 4H).

Compound 5. A solution of 8.5 g of compound 4 (28.7 mmol) in CH₂Cl₂ was mixed with 7.5 mL of DIPEA (43.0 mmol) and cooled to 0° C. 3.9 g of MsCl was added dropwise. The mixture was stirred at room temperature for 2 hours, washed with water, dried over Na₂SO₄ and evaporated. The product was used without purification. Yield 10.7 g (97%). ¹H NMR (400 MHz, DMSO-d₆) δ7.36-7.27 (m, 5H), 4.43 (s, 2H), 4.31-4.29 (m, 2H), 3.67-3.65 (m, 2H), 3.56-3.54 (m, 2H), 3.49-3.46 (m, 2H), 3.41 (t, J=6.5 Hz, 2H), 3.36 (t, J=6.6 Hz, 2H), 3.17 (s, 3H), 1.54-1.48 (m, 4H), 1.33-1.27 (m, 4H).

Compound 6. An oven-dried 250 mL flask with stirrer was charged with sodium hydride (60% dispersion in mineral oil, 2.87 g, 71.7 mmol). The flask was sealed with a septum, evacuated and back-filled with nitrogen (×3). Anhydrous DMF (50 mL) followed by 1,4-butanediol (10.1 g, 86 mmol) were added and the reaction mixture stirred at 25° C. for 2 hr. To the mixture was added a solution of compound 5 (10.7 g, 28.7 mmol) in anhydrous DMF (100 mL). The solution was warmed to 50° C. and stirred for 24 hr. The reaction mixture was cooled to r.t. and concentrated in vacuo. The residue was partitioned between H₂O (400 mL) and Et₂O (500 mL). The organic phase was dried over Na₂SO₄ and concentrated in vacuo. Yield 11.1 g (97%) as viscous oil. ¹H NMR (400 MHz, DMSO-d₆) δ 7.34-7.27 (m, 5H), 4.48 (s, 2H), 3.65-3.42 (m, 17H), 1.69-1.53 (m, 8H), 1.39-1.323 (m, 4H).

Compound 7. Jones reagent (prepared from 8.41 g (84 mmol) of CrO₃, 150 mL of water and 15 mL of conc.H₂SO₄) was added to the solution of compound 6 (11.1 g, 28 mmol) in acetone (300 mL) at 0° C. The reaction mixture was stirred at room temperature overnight; excess of oxidative reagent was neutralized with i-PrOH (50 mL). Solution was concentrated, diluted with water (400 mL) and extracted with CH₂Cl₂ (3×250 mL). Organic phase was dried over Na₂SO₄, solvent was removed in vacuo. Compound 7 was used without purification. Yield 10.3 g (89%). LCMS (C18 column 20×2 mm, 2.5 μm, pore size 100 Å, water-acetonitrile+0.1% TFA, gradient 5 to 87% for 3 min, retention time 1.58 min). MS (ESI) m/z 383.6 [MH]+.

Compound 8. 2 mL (27.5 mmol) of SOCl₂ was added to the stirred solution of compound 7 (10.3 g, 25 mmol) in dry methanol (150 mL) at room temperature. After 8 hours reaction mixture was concentrated and purified with column chromatography (SiO₂, ethyl acetate (33%)-hexane (66%)). Yield 5.6 g (53%). ¹H NMR (400 MHz, DMSO-d₆) δ 7.36-7.26 (m, 5H), 4.43 (s, 2H), 3.57 (s, 3H), 3.49-3.34 (m, 14H), 2.33 (t, J=6.4 Hz, 2H), 1.77-1.70 (m, 2H), 1.54-1.45 (m, 4H), 1.34-1.23 (m, 4H).

Compound 9. H₂ gas was passed through a mixture of compound 8 (1.1 g, 2.36 mmol) and 10% Pd/C (0.3 g) in EtOH (20 ml) at r.t. for 12 h. The powder of Pd/C was filtered, and the solvent was removed under reduced pressure to provide the product 9. Yield: 0.89 g (100%). ¹H NMR (400 MHz, DMSO-d₆) δ 4.30 (t, J=5.1 Hz, 1H), 3.58 (s, 3H), 3.51-3.44 (m, 8H), 3.39-3.34 (m, 6H), 2.33 (t, J=6.4 Hz, 2H), 1.77-1.74 (m, 2H), 1.54-1.45 (m, 4H), 1.34-1.23 (m, 4H)

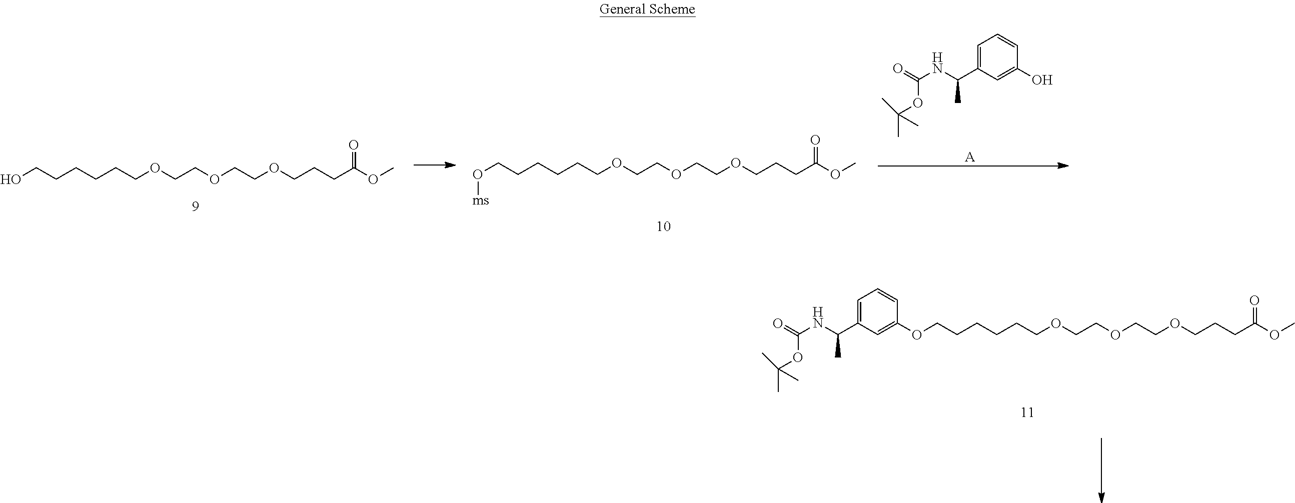

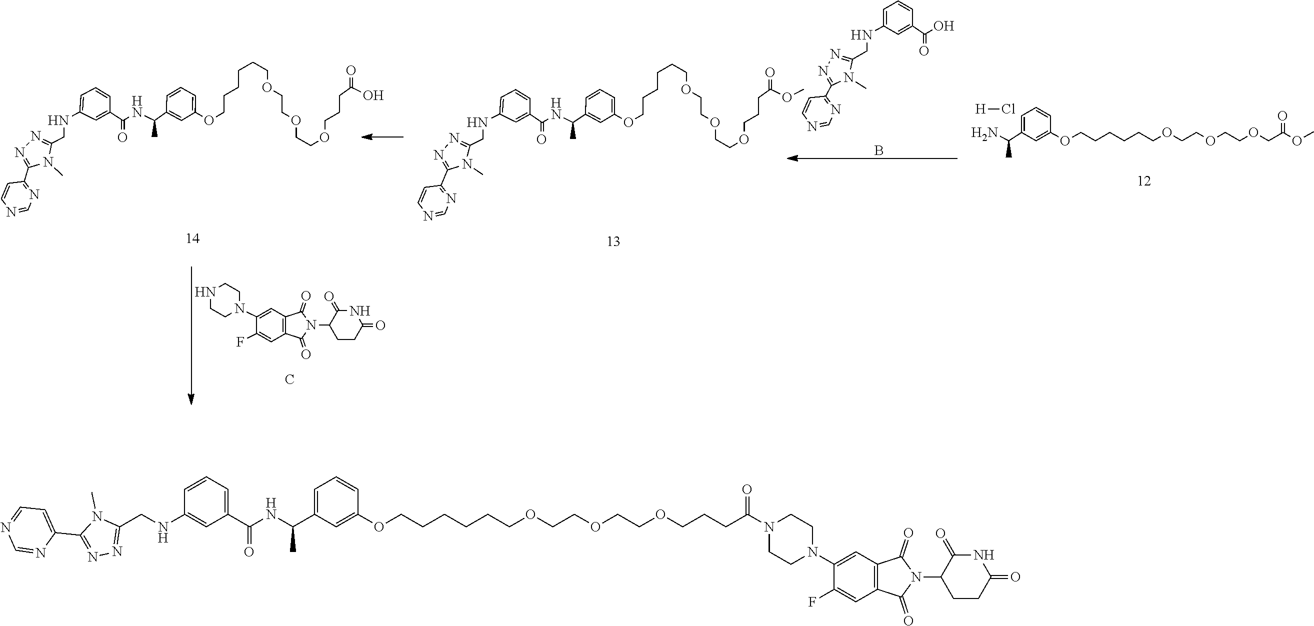

### General Procedures

Compound 10. A solution of compound 9 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 11. A mixture of compound A (2.5 mmol, 1 eq), compound 10 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 11.

Compound 12. A solution of compound 11 (1.2 mmol, 1 eq) in dry DCM (50 mL) with 1.2 ml 3 M HCl in dioxane (5 eq) were stirred for 24 h at r.t. The mixture was evaporated to dryness to give the crude product 12 which was used in the next step without additional purification.

Compound 13. A mixture of compound 12 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq) and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 13.

Compound 14. To a solution of compound 13 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D178. A mixture of compound 14 (0.1 mmol, 1 eq), compound C (or other recruiter) (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was purified by HPLC in neutral conditions (YMC-Pack ODS-AQ 250×20 mml, S-10 μm, 12 nm, gradient water-acetonitrile).

### Synthesis According to the General Procedures

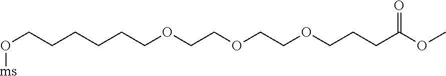

Compound 10. Methyl 3,10,13,16-tetraoxa-2-thiaicosan-20-oate 2,2-dioxide. Yield 97%.

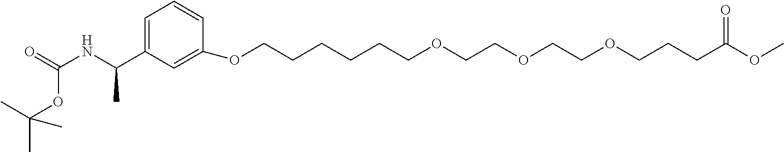 [SMILES: COC(=O)CCCOCCOCCOCCCCCCOc1cccc([C@@H](C)NC(=O)OC(C)(C)C)c1]

Compound 11. (R)-methyl 4-(2-(2-(6-(3-(1-(tert-butoxycarbonylamino)ethyl)phenoxy) hexyloxy)ethoxy)ethoxy)butanoate. Yield 86%. Colorless oil 98%. LCMS (C18 column 20×2 mm, 2.5 μm, pore size 100 Å, water-acetonitrile+0.1% TFA, gradient 5 to 87% for 3 min, retention time 1.87 min). MS (ESI) m/z 526.5 [MH]+. 1H NMR (400 MHz, DMSO-d6) δ 7.30 (d, J=8.3 Hz, 1H), 7.18 (t, J=7.8 Hz, 1H), 6.84-6.82 (m, 2H), 6.74 (dd, J=7.8, 1.6 Hz, 1H), 4.56 (m 1H), 3.92 (t, J=6.4 Hz, 2H), 3.57 (s, 3H), 3.38 (t, J=6.4 Hz, 4H), 2.33 (t, J=7.4 Hz, 2H), 1.77-1.66 (m, 4H), 1.54-1.47 (m, 2H), 1.43-1.21 (m, 16H).

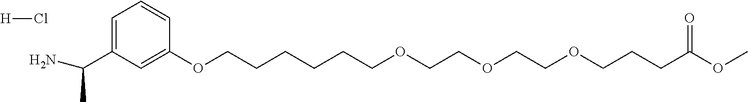

Compound 12. (R)-methyl 4-(2-(2-(6-(3-(1-aminoethyl)phenoxy)hexyloxy)ethoxy)ethoxy)butanoate hydrochloride. Yield 99%. LCMS (C18 column 20×2 mm, 2.5 μm, pore size 100 Å, water-acetonitrile+0.1% TFA, gradient 5 to 87% for 3 min, retention time 1.13 min). MS (ESI) m/z 426.4 [MH]+.

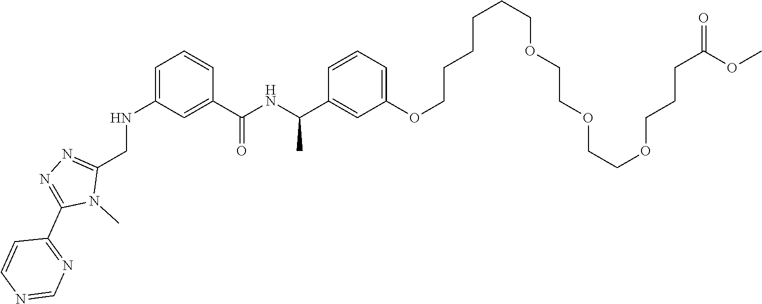 [SMILES: COC(=O)CCCOCCOCCOCCCCCCOc1cccc([C@@H](C)NC(=O)c2cccc(NCc3nnc(-c4ccncn4)n3C)c2)c1]

Compound 13. (R)-methyl 4-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoate. Yield 95%. LCMS (C18 column 20×2 mm, 2.5 μm, pore size 100 Å, water-acetonitrile+0.1% TFA, gradient 5 to 87% for 3 min, retention time 1.52 min). MS (ESI) m/z 718.5 [MH]+.

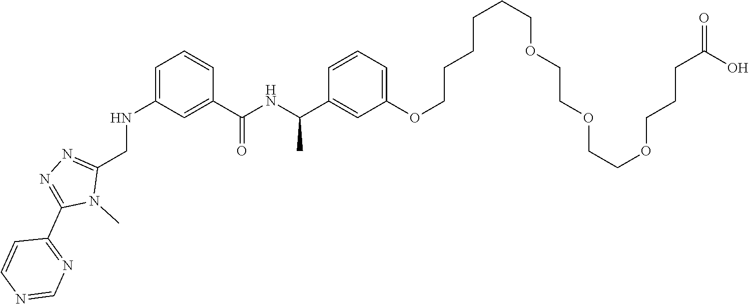 [SMILES: C[C@@H](NC(=O)c1cccc(NCc2nnc(-c3ccncn3)n2C)c1)c1cccc(OCCCCCCOCCOCCOCCCC(=O)O)c1]

Compound 14. (R)-4-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoic acid. Yield 81%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 704.7[MH]+.

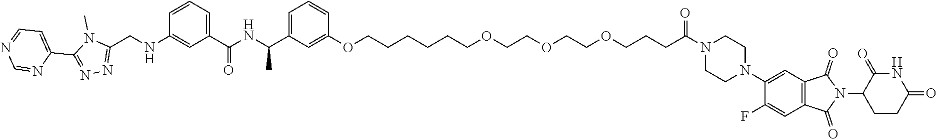 [SMILES: C[C@@H](NC(=O)c1cccc(NCc2nnc(-c3ccncn3)n2C)c1)c1cccc(OCCCCCCOCCOCCOCCCC(=O)N2CCN(c3cc4c(cc3F)C(=O)N(C3CCC(=O)NC3=O)C4=O)CC2)c1]

D178. N-((1R)-1-(3-((6-(2-(2-(4-(4-(2-(2,6-dioxopiperidin-3-yl)-6-fluoro-1,3-dioxoisoindolin-5-yl)piperazin-1-yl)-4-oxobutoxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide. Yield 33%. LCMS (C18 column 100×4.6 mm, 5.0 μm, pore size 100 Å, water-acetonitrile+0.1% TFA, gradient 5 to 87% for 10 min, retention time 6.36 min). MS (ESI) m/z 1047.1[MH]+. ¹H NMR (400 MHz, DMSO-d₆) δ 11.09 (s, 1H), 9.32 (d, J=1.4 Hz, 1H), 8.96 (d, J=5.3 Hz, 1H), 8.54 (d, J=8.2 Hz, 1H), 8.17 (dd, J=5.3, 1.4 Hz, 1H), 7.74 (d, J=11.3 Hz, 1H), 7.46 (d, J=7.3 Hz, 1H), 7.18 (m, 3H), 7.10 (d, J=7.7 Hz, 1H), 6.91 (m, 3H), 6.75 (m, 1H), 6.44 (t, J=5.7 Hz, 1H), 5.10 (m, 2H), 4.57 (d, J=5.7 Hz, 2H), 4.07 (s, 3H), 3.92 (t, J=6.4 Hz, 2H), 3.61 (s, 4H), 3.48 (m, 8H), 3.38 (m, 4H), 3.23 (m, 4H), 2.88 (m, 1H), 2.59 (m, 1H), 2.38 (m, 3H), 2.03 (m, 1H), 1.71 (m, 4H), 1.45 (m, 5H), 1.35 (m, 4H).

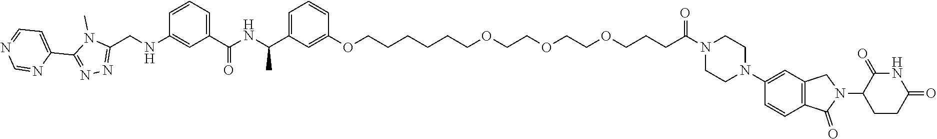 [SMILES: C[C@@H](NC(=O)c1cccc(NCc2nnc(-c3ccncn3)n2C)c1)c1cccc(OCCCCCCOCCOCCOCCCC(=O)N2CCN(c3ccc4c(c3)CN(C3CCC(=O)NC3=O)C4=O)CC2)c1]

D179. N-((1R)-1-(3-((6-(2-(2-(4-(4-(2-(2,6-dioxopiperidin-3-yl)-1-oxoisoindolin-5-yl)piperazin-1-yl)-4-oxobutoxy)ethoxy)ethoxy)hexyl)oxy)phenyl)ethyl)-3-(((4-methyl-5-(pyrimidin-4-yl)-4H-1,2,4-triazol-3-yl)methyl)amino)benzamide Yield 43%. LCMS (C18 column 100×4.6 mm, 5.0 μm, pore size 100 Å, water-acetonitrile+0.1% TFA, gradient 5 to 87% for 10 min, retention time 5.79 min). MS (ESI) m/z 1015.2[MH]+. ¹H NMR (400 MHz, DMSO-d₆) δ 10.92 (s, 1H), 9.32 (d, J=1.4 Hz, 1H), 8.96 (d, J=5.3 Hz, 1H), 8.54 (d, J=8.1 Hz, 1H), 8.17 (dd, J=5.3, 1.4 Hz, 1H), 7.53 (d, J=8.4 Hz, 1H), 7.17 (m, 3H), 7.10 (d, J=7.6 Hz, 1H), 7.05 (m, 2H), 6.91 (m, 3H), 6.75 (m, 1H), 6.44 (t, J=5.6 Hz, 1H), 5.06 (m, 2H), 4.57 (d, J=5.6 Hz, 2H), 4.26 (m, 2H), 4.06 (s, 3H), 3.91 (t, J=6.4 Hz, 2H), 3.59 (s, 4H), 3.48 (m, 8H), 3.36 (m, 4H), 3.26 (m, 4H), 2.88 (m, 1H), 2.60 (s, 1H), 2.38 (m, 3H), 1.94 (m, 1H), 1.70 (m, 4H), 1.45 (m, 5H), 1.34 (m, 4H).

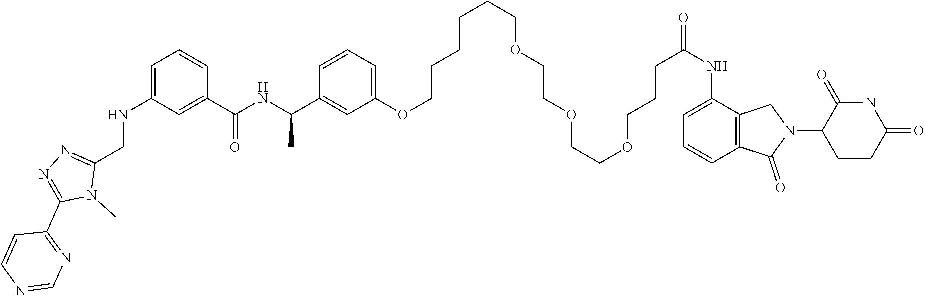 [SMILES: C[C@@H](NC(=O)c1cccc(NCc2nnc(-c3ccncn3)n2C)c1)c1cccc(OCCCCCCOCCOCCOCCCC(=O)Nc2cccc3c2CN(C2CCC(=O)NC2=O)C3=O)c1]

D163. N-((1R)-1-(3-(6-(2-(2-(4-(2-(2,6-dioxopiperidin-3-yl)-1-oxoisoindolin-4-ylamino)-4-oxobutoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 46%. LCMS (C18 column 100×4.6 mm, 5.0 μm, pore size 100 Å, water-acetonitrile+0.1% TFA, gradient 5 to 87% for 10 min, retention time 5.76 min). MS (ESI) m/z 945.8[MH]+. ¹H NMR (400 MHz, DMSO-d₆) δ 11.01 (s, 1H), 9.77 (s, 1H), 9.33 (s, 1H), 8.97 (d, J=5.3 Hz, 1H), 8.55 (d, J=8.1 Hz, 1H), 8.18 (dd, J=5.3, 1.3 Hz, 1H), 7.80 (dd, J=7.1, 1.5 Hz, 1H), 7.50-7.45 (m, 2H), 7.21-7.09 (m, 4H), 6.93-6.89 (m, 3H), 6.75 (d, J=9.3 Hz, 1H), 6.46 (t, J=5.7 Hz, 1H), 5.16-5.07 (m, 2H), 4.58 (d, J=17.6 Hz, 2H), 4.41-4.30 (m, 2H), 4.07 (s, 3H), 3.91 (t, J=6.5 Hz, 2H), 3.52-3.43 (m, 10H), 3.35 (t, J=6.5 Hz, 2H), 2.96-2.89 (m, 1H), 2.67-2.54 (m, 1H), 2.41 (t, J=7.6 Hz, 2H), 2.36-2.28 (m, 1H), 2.05-2.00 (m, 1H), 1.85-1.79 (m, 2H), 1.71-1.64 (m, 2H), 1.51-1.29 (m, 9H).

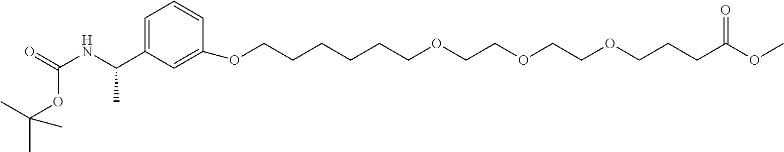 [SMILES: COC(=O)CCCOCCOCCOCCCCCCOc1cccc([C@H](C)NC(=O)OC(C)(C)C)c1]

Compound 11. (S)-methyl 4-(2-(2-(6-(3-(1-(tert-butoxycarbonylamino)ethyl)phenoxy) hexyloxy)ethoxy)ethoxy)butanoate. Yield 86%. Colorless oil 98%. LCMS (C18 column 20×2 mm, 2.5 μm, pore size 100 Å, water-acetonitrile+0.1% TFA, gradient 5 to 87% for 3 min, retention time 1.87 min). MS (ESI) m/z 526.5 [MH]+. ¹H NMR (400 MHz, DMSO-d₆) δ 7.30 (d, J=8.3 Hz, 1H), 7.18 (t, J=7.8 Hz, 1H), 6.84-6.82 (m, 2H), 6.74 (dd, J=7.8, 1.6 Hz, 1H), 4.56 (m 1H), 3.92 (t, J=6.4 Hz, 2H), 3.57 (s, 3H), 3.38 (t, J=6.4 Hz, 4H), 2.33 (t, J=7.4 Hz, 2H), 1.77-1.66 (m, 4H), 1.54-1.47 (m, 2H), 1.43-1.21 (m, 16H)

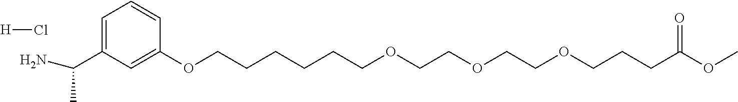

Compound 12. ((R)-methyl 4-(2-(2-(6-(3-(1-aminoethyl)phenoxy)hexyloxy)ethoxy)ethoxy)butanoate hydrochloride. Yield 99%. LCMS (C18 column 20×2 mm, 2.5 μm, pore size 100 Å, water-acetonitrile+0.1% TFA, gradient 5 to 87% for 3 min, retention time 1.13 min). MS (ESI) m/z 426.4 [MH]+.

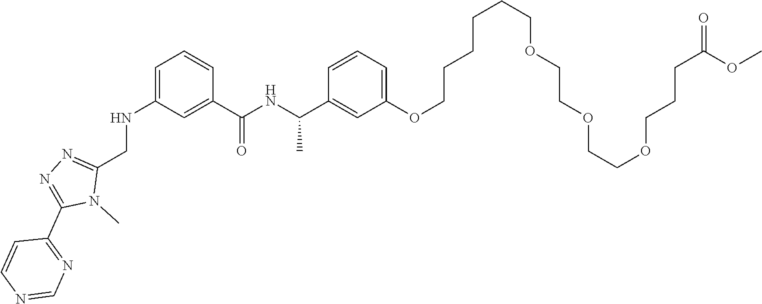 [SMILES: COC(=O)CCCOCCOCCOCCCCCCOc1cccc([C@H](C)NC(=O)c2cccc(NCc3nnc(-c4ccncn4)n3C)c2)c1]

Compound 13. (S)-methyl 4-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoate. Yield 95%. LCMS (C18 column 20×2 mm, 2.5 μm, pore size 100 Å, water-acetonitrile+0.1% TFA, gradient 5 to 87% for 3 min, retention time 1.52 min). MS (ESI) m/z 718.5 [MH]+.

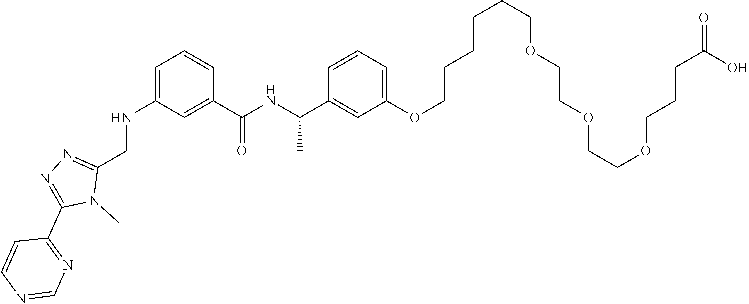 [SMILES: C[C@H](NC(=O)c1cccc(NCc2nnc(-c3ccncn3)n2C)c1)c1cccc(OCCCCCCOCCOCCOCCCC(=O)O)c1]

Compound 14. (S)-4-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoic acid. Yield 81%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 704.7[MH]+.

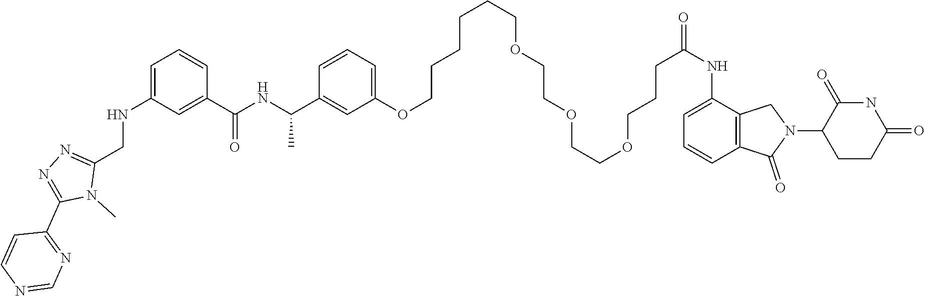 [SMILES: C[C@H](NC(=O)c1cccc(NCc2nnc(-c3ccncn3)n2C)c1)c1cccc(OCCCCCCOCCOCCOCCCC(=O)Nc2cccc3c2CN(C2CCC(=O)NC2=O)C3=O)c1]

D154. N-((1S)-1-(3-(6-(2-(2-(4-(2-(2,6-dioxopiperidin-3-yl)-1-oxoisoindolin-4-ylamino)-4-oxobutoxy)ethoxy)ethoxy)hexyloxy)phenyl)ethyl)-3-((4-methyl-5-(pyrimidin-4-yl)-4H-1,2,4-triazol-3-yl)methylamino)benzamide. LCMS (C18 column 100×4.6 mm, 5.0 μm, pore size 100 Å, water-acetonitrile+0.1% TFA, gradient 5 to 87% for 10 min, retention time 5.76 min). MS (ESI) m/z 945.8[MH]+. ¹H NMR (400 MHz, DMSO-d₆) δ 11.00 (s, 1H), 9.77 (s, 1H), 9.32 (s, 1H), 8.97 (d, J=5.3 Hz, 1H), 8.56 (d, J=8.1 Hz, 1H), 8.16 (dd, J=5.3, 1.3 Hz, 1H), 7.82 (dd, J=7.1, 1.5 Hz, 1H), 7.50-7.45 (m, 2H), 7.21-7.09 (m, 4H), 6.93-6.89 (m, 3H), 6.76 (d, J=9.3 Hz, 1H), 5.16-5.07 (m, 2H), 4.57 (s, 2H), 4.41-4.30 (m, 2H), 4.07 (s, 3H), 3.91 (t, J=6.5 Hz, 2H), 3.52-3.43 (m, 10H), 3.35 (t, J=6.5 Hz, 2H), 2.96-2.89 (m, 1H), 2.67-2.54 (m, 2H), 2.41 (t, J=7.6 Hz, 2H), 2.36-2.28 (m, 1H), 2.05-2.00 (m, 1H), 1.85-1.79 (m, 2H), 1.71-1.64 (m, 2H), 1.51-1.29 (m, 9H).

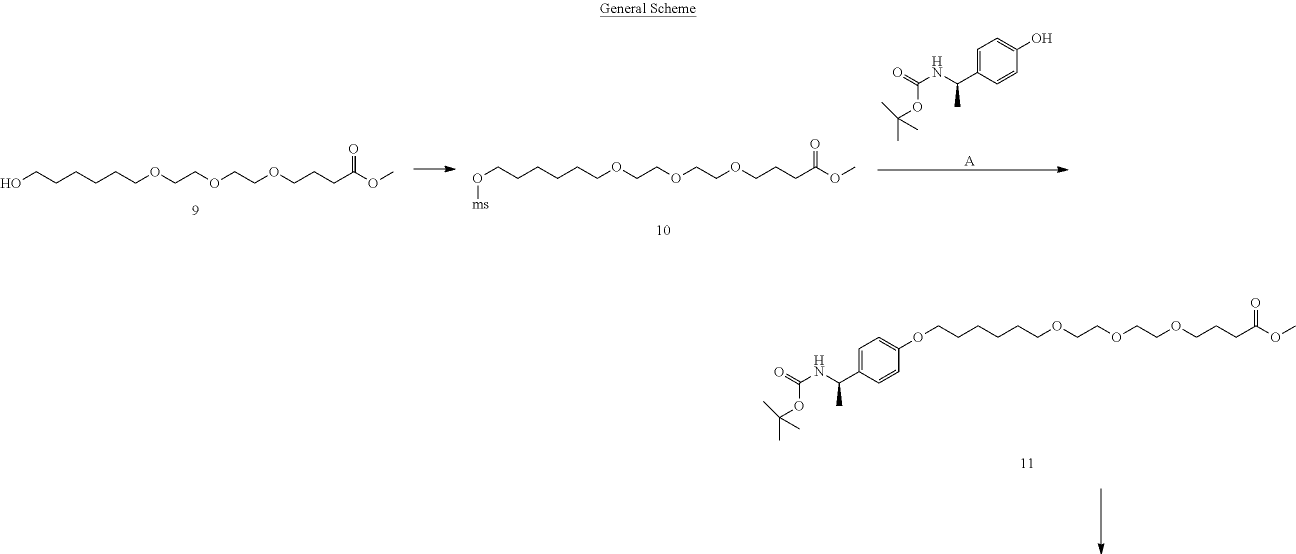

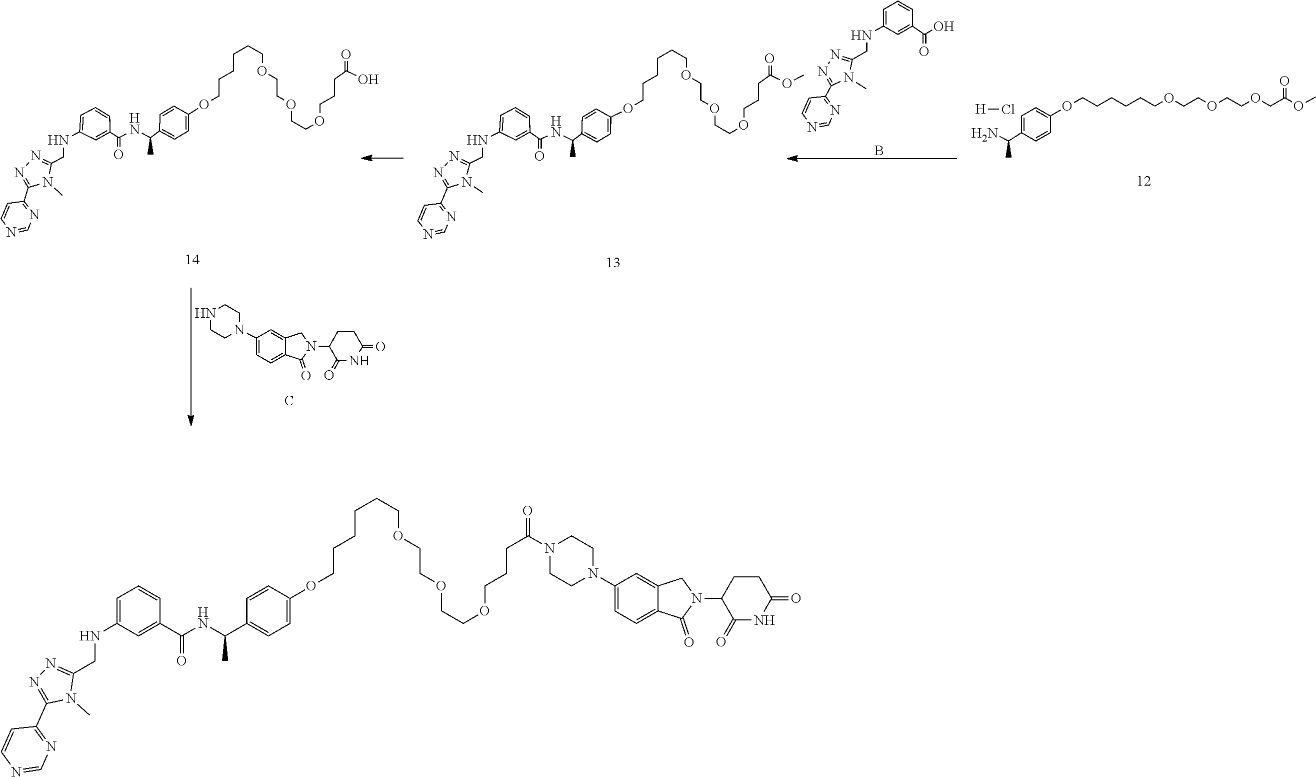

### General Procedures

Compound 10. A solution of compound 9 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 11. A mixture of compound A (2.5 mmol, 1 eq), compound 10 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 11.

Compound 12. A solution of compound 11 (1.2 mmol, 1 eq) in dry DCM (50 mL) with 1.2 ml 3 M HCl in dioxane (5 eq) were stirred for 24 h at r.t. The mixture was evaporated to dryness to give the crude product 12 which was used in the next step without additional purification.

Compound 13. A mixture of compound 12 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq) and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 13.

Compound 14. To a solution of compound 13 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D124. A mixture of compound 14 (0.1 mmol, 1 eq), compound C (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was purified by HPLC in acidic conditions (YMC-Pack ODS-AQ 250×20 mml, S-10 μm, 12 nm, gradient water-acetonitrile).

### Synthesis According General Procedures

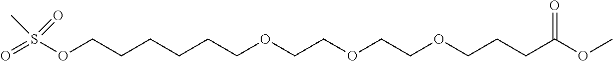 [SMILES: COC(=O)CCCOCCOCCOCCCCCCOS(C)(=O)=O]

Compound 10. Methyl 3,10,13,16-tetraoxa-2-thiaicosan-20-oate 2,2-dioxide. Yield 97%

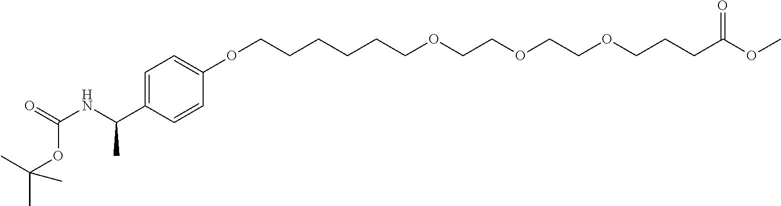 [SMILES: COC(=O)CCCOCCOCCOCCCCCCOc1ccc([C@@H](C)NC(=O)OC(C)(C)C)cc1]

Compound 11. (R)-methyl 4-(2-(2-(6-(4-(1-(tert-butoxycarbonylamino)ethyl)phenoxy) hexyloxy)ethoxy)ethoxy)butanoate. Yield 86%. Colorless oil 98%. LCMS (C18 column 20×2 mm, 2.5 μm, pore size 100 Å, water-acetonitrile+0.1% TFA, gradient 5 to 87% for 3 min, retention time 1.90 min). MS (ESI) m/z 526.5 [MH]+. ¹H NMR (400 MHz, DMSO-d₆) δ 7.25 (d, J=8.9 Hz, 1H), 7.18 (d, J=8.6 Hz, 2H), 6.83 (d, J=8.6 Hz, 2H 2H), 4.56 (m 1H), 3.92 (t, J=6.4 Hz, 2H), 3.57 (s, 3H), 3.49-3.45 (m, 8H), 3.38 (t, J=7.4 Hz, 4H), 2.33 (t, J=7.4 Hz, 2H), 1.77-1.66 (m, 4H), 1.54-1.47 (m, 2H), 1.43-1.21 (m, 16H).

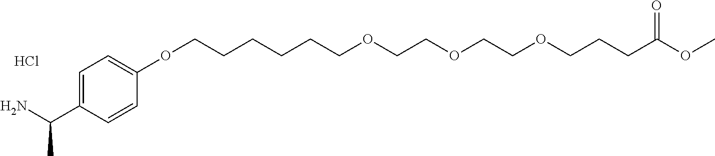 [SMILES: Cl ; COC(=O)CCCOCCOCCOCCCCCCOc1ccc([C@@H](C)N)cc1]

Compound 12. (R)-methyl 4-(2-(2-(6-(4-(1-aminoethyl)phenoxy)hexyloxy)ethoxy)ethoxy)butanoate hydrochloride. Yield 99%. LCMS (C18 column 20×2 mm, 2.5 μm, pore size 100 Å, water-acetonitrile+0.1% TFA, gradient 5 to 87% for 3 min, retention time 1.15 min). MS (ESI) m/z 426.4 [MH]+.

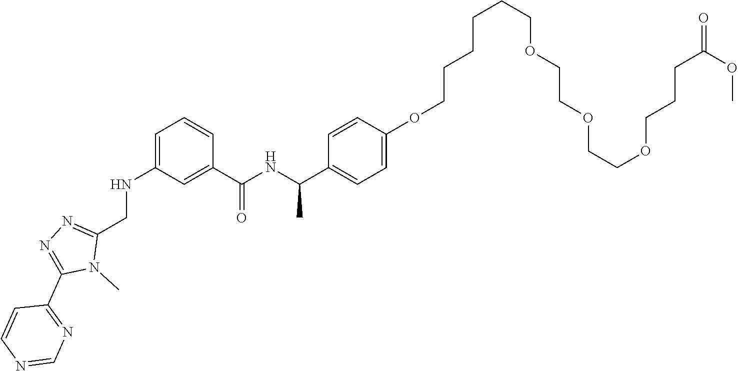 [SMILES: COC(=O)CCCOCCOCCOCCCCCCOc1ccc([C@@H](C)NC(=O)c2cccc(NCc3nnc(-c4ccncn4)n3C)c2)cc1]

Compound 13. (R)-methyl 4-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoate. Yield 95%. LCMS (C18 column 20×2 mm, 2.5 μm, pore size 100 Å, water-acetonitrile+0.1% TFA, gradient 5 to 87% for 3 min, retention time 1.53 min). MS (ESI) m/z 718.5 [MH]+.

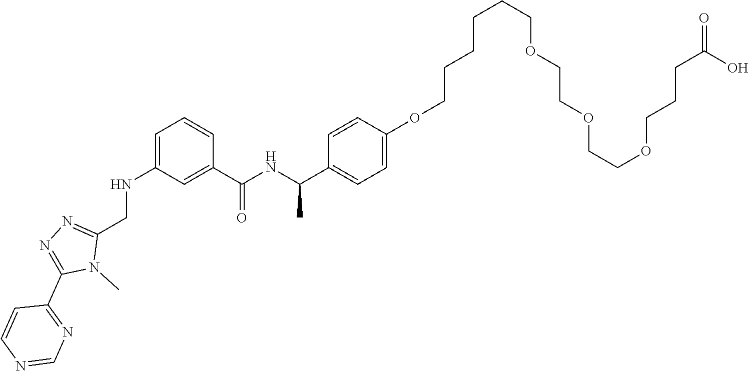 [SMILES: C[C@@H](NC(=O)c1cccc(NCc2nnc(-c3ccncn3)n2C)c1)c1ccc(OCCCCCCOCCOCCOCCCC(=O)O)cc1]

Compound 14. (R)-4-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)butanoic acid. Yield 81%. LCMS (C18 column 20×2 mm, 2.5 μm, pore size 100 Å, water-acetonitrile+0.1% TFA, gradient 5 to 87% for 3 min, retention time 1.37 min). MS (ESI) m/z 704.7[MH]+.

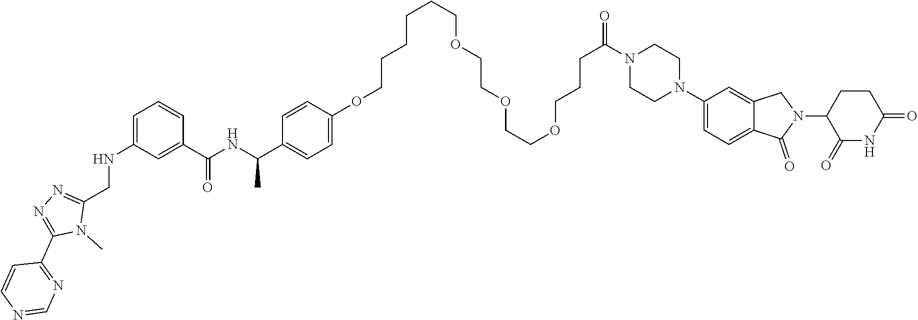 [SMILES: C[C@@H](NC(=O)c1cccc(NCc2nnc(-c3ccncn3)n2C)c1)c1ccc(OCCCCCCOCCOCCOCCCC(=O)N2CCN(c3ccc4c(c3)CN(C3CCC(=O)NC3=O)C4=O)CC2)cc1]

D124. N-((1R)-1-(4-(6-(2-(2-(4-(4-(2-(2,6-dioxopiperidin-3-yl)-1-oxoisoindolin-5-yl)piperazin-1-yl)-4-oxobutoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 61%. LCMS (C18 column 100×4.6 mm, 5.0 μm, pore size 100 Å, water-acetonitrile+0.1% TFA, gradient 5 to 87% for 10 min, retention time 5.70 min). MS (ESI) m/z 1015.1 [MH]+. ¹H NMR (400 MHz, DMSO-d₆) δ 10.92 (s, 1H), 9.33 (s, 1H), 8.97 (d, J=5.3 Hz, 1H), 8.51 (d, J=8.2 Hz, 1H), 8.18 (dd, J=5.3, 1.3 Hz, 1H), 7.54 (d, J=8.3 Hz, 1H), 7.27 (d, J=8.6 Hz, 2H), 7.18-7.14 (m, 2H), 7.09-7.04 (m, 3H), 6.90-6.83 (m, 3H), 6.43 (t, J=5.5 Hz, 1H), 5.10-5.02 (m, 2H), 4.57 (d, J=5.7 Hz, 2H), 4.35-4.18 (m, 2H), 4.06 (s, 3H), 3.90 (t, J=6.5 Hz, 2H), 3.59 (br, 4H), 3.50-3.45 (m, 9H), 3.41-3.35 (m, 4H), 3.27-3.21 (m, 2H), 2.94-2.85 (m, 1H), 2.60-2.54 (m, 1H), 2.41-2.31 (m, 3H), 1.98-1.93 (m, 1H), 1.77-1.63 (m, 4H), 1.52-1.30 (m, 10H).

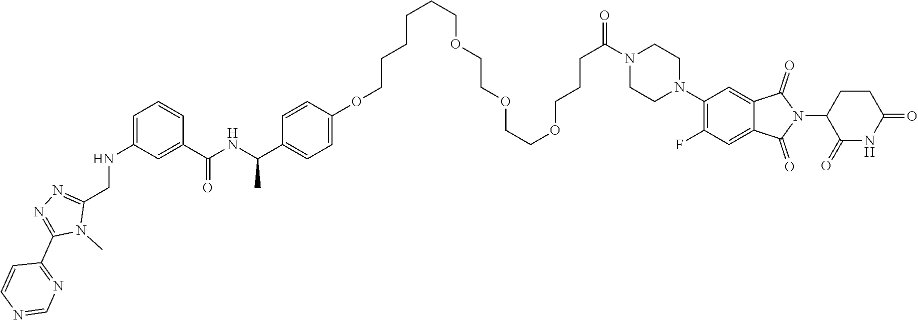 [SMILES: C[C@@H](NC(=O)c1cccc(NCc2nnc(-c3ccncn3)n2C)c1)c1ccc(OCCCCCCOCCOCCOCCCC(=O)N2CCN(c3cc4c(cc3F)C(=O)N(C3CCC(=O)NC3=O)C4=O)CC2)cc1]

D125. N-((1R)-1-(4-(6-(2-(2-(4-(4-(2-(2,6-dioxopiperidin-3-yl)-6-fluoro-1,3-dioxoisoindolin-5-yl)piperazin-1-yl)-4-oxobutoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 51%. LCMS (C18 column 100×4.6 mm, 5.0 μm, pore size 100 Å, water-acetonitrile+0.1% TFA, gradient 5 to 87% for 10 min, retention time 6.26 min). MS (ESI) m/z 1047.3[MH]+. ¹H NMR (400 MHz, DMSO-d₆) δ 11.09 (s, 1H), 9.32 (s, 1H), 8.97 (d, J=5.3 Hz, 1H), 8.49 (d, J=8.3 Hz, 1H), 8.17 (dd, J=5.3, 1.3 Hz, 1H), 7.75 (d, J=11.3 Hz, 1H), 7.46 (d, J=7.4 Hz, 1H), 7.28 (d, J=8.7 Hz, 2H) 7.18-7.07 (m, 3H), 6.90-6.83 (m, 3H), 6.43 (t, J=5.7 Hz, 1H), 5.13-5.07 (m, 2H), 4.57 (d, J=5.6 Hz, 2H), 4.07 (s, 3H), 3.90 (t, J=6.5 Hz, 2H), 3.61 (br, 4H), 3.50-3.45 (m, 8H), 3.42-3.35 (m, 4H), 3.25 (br, 2H), 3.20 (br, 2H), 2.93-2.84 (m, 1H), 2.67-2.54 (m, 2H), 2.40 (t, J=7.6 Hz, 2H), 2.06-2.00 (m, 1H), 1.77-1.63 (m, 4H), 1.52-1.30 (m, 9H).

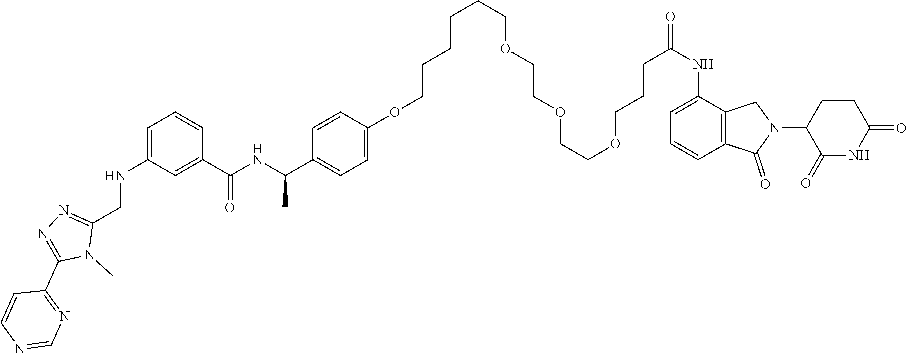 [SMILES: C[C@@H](NC(=O)c1cccc(NCc2nnc(-c3ccncn3)n2C)c1)c1ccc(OCCCCCCOCCOCCOCCCC(=O)Nc2cccc3c2CN(C2CCC(=O)NC2=O)C3=O)cc1]

D127. N-((1R)-1-(4-(6-(2-(2-(4-(2-(2,6-dioxopiperidin-3-yl)-1-oxoisoindolin-4-ylamino)-4-oxobutoxy)ethoxy)ethoxy)hexyloxy)phenyl)ethyl)-3-((4-methyl-5-(pyrimidin-4-yl)-4H-1,2,4-triazol-3-yl)methylamino)benzamide. LCMS (C18 column 100×4.6 mm, 5.0 μm, pore size 100 Å, water-acetonitrile+0.1% TFA, gradient 5 to 87% for 10 min, retention time 5.73 min). MS (ESI) m/z 946.1[MH]+. ¹H NMR (400 MHz, DMSO-d₆) δ 11.00 (s, 1H), 9.76 (s, 1H), 9.33 (s, 1H), 8.96 (d, J=5.3 Hz, 1H), 8.51 (d, J=8.3 Hz, 1H), 8.18 (dd, J=5.3, 1.3 Hz, 1H), 7.82 (d, J=11.3 Hz, 1H), 7.50-7.44 (m, 2H), 7.28 (d, J=8.7 Hz, 2H) 7.18-7.14 (m, 2H), 7.09-7.07 (m, 1H), 6.90-6.83 (m, 3H), 6.43 (t, J=5.7 Hz, 1H), 5.18-5.05 (m, 2H), 4.57 (d, J=5.6 Hz, 2H), 4.41-4.30 (m, 2H), 4.07 (s, 3H), 3.90 (t, J=6.5 Hz, 2H), 3.52-3.42 (m, 10H), 3.36 (t, J=6.5 Hz, 2H), 2.95-2.86 (m, 1H), 2.67-2.54 (m, 2H), 2.40 (t, J=7.6 Hz, 2H), 2.36-2.29 (m, 1H), 2.05-1.99 (m, 1H), 1.85-1.79 (m, 2H), 1.70-1.63 (m, 2H), 1.51-1.30 (m, 10H).

### Example 24. Preparation of D130, D131, D136, D137, D142, and D195

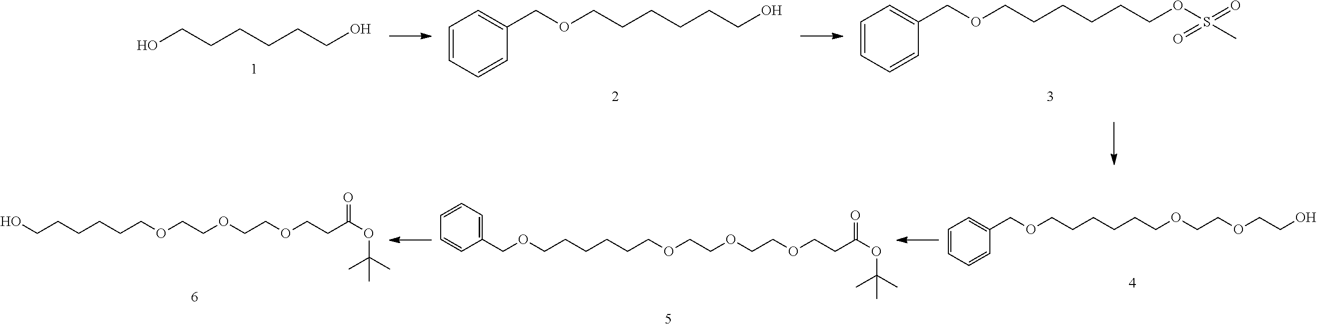 [SMILES: OCCCCCCO ; OCCCCCCOCc1ccccc1 ; CS(=O)(=O)OCCCCCCOCc1ccccc1 ; CC(C)(C)OC(=O)CCOCCOCCOCCCCCCO ; CC(C)(C)OC(=O)CCOCCOCCOCCCCCCOCc1ccccc1 ; OCCOCCOCCCCCCOCc1ccccc1]

Compound 2. A mixture of compounds 1 (65.8 g, 631 mmol, 4 eq), benzyl chloride (20.0 g, 158 mmol, 1 eq.), TBAB (0.2 g) and 50% aqua solution of NaOH (25.3 g, 631 mmol, 1 eq) was stirred at 100° C. for 12 h before been poured in water (200 ml). The product was extracted with Et₂O (3×150 ml), the organic layers were dried over Na₂SO₄, the solvent was removed under reduced pressure. The residue was purified by column chromatography on silica gel, using hexane/EtOAc 1:1 as eluent, to provide the product 2. Yield 85%. ¹H NMR (400 MHz, DMSO-d₆) δ 7.36-7.27 (m, 5H), 4.43 (s, 2H), 4.33 (t, J=4.3 Hz, 1H), 3.42-3.37 (m, 4H), 1.56-1.49 (m, 2H), 1.44-1.37 (m, 2H), 1.35-1.25 (m, 4H).

Compound 3. Solution of compound 2 (15.8 g, 75.85 mmol) in CH₂Cl₂ was mixed with 19.8 mL of DIPEA (113.77 mmol) and cooled to 0° C. 10.42 g of MsCl was added dropwise. The mixture was stirred at room temperature for 2 hours, washed with water, dried over Na₂SO₄ and evaporated. The product was used without purification. Yield 21.9 g (98%). ¹H NMR (400 MHz, DMSO-d₆) δ 7.36-7.27 (m, 5H), 4.44 (s, 2H), 4.19 (t, J=6.2 Hz, 2H), 3.42 (t, J=6.4 Hz, 2H), 3.15 (s, 3H), 1.69-1.62 (m, 2H), 1.56-1.51 (m, 2H), 1.41-1.30 (m, 4H).

Compound 4. An oven-dried 500 mL flask with stirrer was charged with sodium hydride (60% dispersion in mineral oil, 7.5 g, 187 mmol). The flask was sealed with a septum, evacuated and back-filled with argon (3 times). Anhydrous DMF (100 mL) followed by diethylene glycol (24.1 g, 227.5 mmol) were added and the reaction mixture stirred at 25° C. for 2 hr. Solution of 5-benzyloxyhexthyl 4-methanesulfonate 3 (21.7 g, 75.8 mmol) in anhydrous DMF (200 mL) was added to the mixture. The solution was warmed to 50° C. and stirred for 18 hr before been cooled to r.t. and concentrated in vacuo. The residue was partitioned between H₂O (400 mL) and Et₂O (500 mL). The organic layer was dried over Na₂SO₄ and concentrated in vacuo. The crude yellow oil was then purified by flash chromatography (200 g SiO₂, EtOAc and hexane (50/50)) providing compound 4 (8.5 g, 38% yield) as viscous oil. ¹H NMR (400 MHz, DMSO-d₆) δ 7.36-7.27 (m, 5H), 4.55 (t, J=5.5 Hz, 1H), 4.44 (s, 2H), 3.51-3.45 (m, 6H), 3.42-3.39 (m, 4H), 3.36 (t, J=6.6 Hz, 2H), 1.55-1.46 (m, 4H), 1.33-1.27 (m, 4H).

Compound 5. To a solution of 15.5 g of compound 4 (52.4 mmol) and 10.0 g of t-buthyl actylate (78.7 mmol) in THF catalytic amount of NaH was added. The mixture was stirred at room temperature for 16 hours before been evaporated. The product was purified by column chromatography (hexane-Et2O, 5 to 1 ratio). Yield 17.2 g (77%). ¹H NMR (400 MHz, DMSO-d₆) δ7.36-7.27 (m, 5H), 4.43 (s, 2H), 3.58 (t, J=6.2 Hz, 2H), 3.48-3.35 (m, 12H), 2.40 (t, J=6.2 Hz, 2H), 1.55-1.485 (m, 4H), 1.39 (s, 9H), 1.33-1.27 (m, 4H).

Compound 6. H₂ gas was passed through a mixture of compound 5 (17.2 g, 40.6 mmol) and 10% Pd/C (2.15 g) in EtOH (50 ml) at r.t. for 12 h. The powder of Pd/C was filtered, and the solvent was removed under reduced pressure to provide the product 6. Yield: 13 g (95%).

¹H NMR (400 MHz, DMSO-d₆) δ 4.30 (t, J=5.1 Hz, 1H), 3.58 (t, J=6.2 Hz 2H), 3.49-3.45 (m, 8H), 3.38-3.34 (m, 4H), 2.41 (t, J=6.4 Hz, 2H), 1.50-1.38 (m, 13H), 1.28-1.26 (m, 4H).

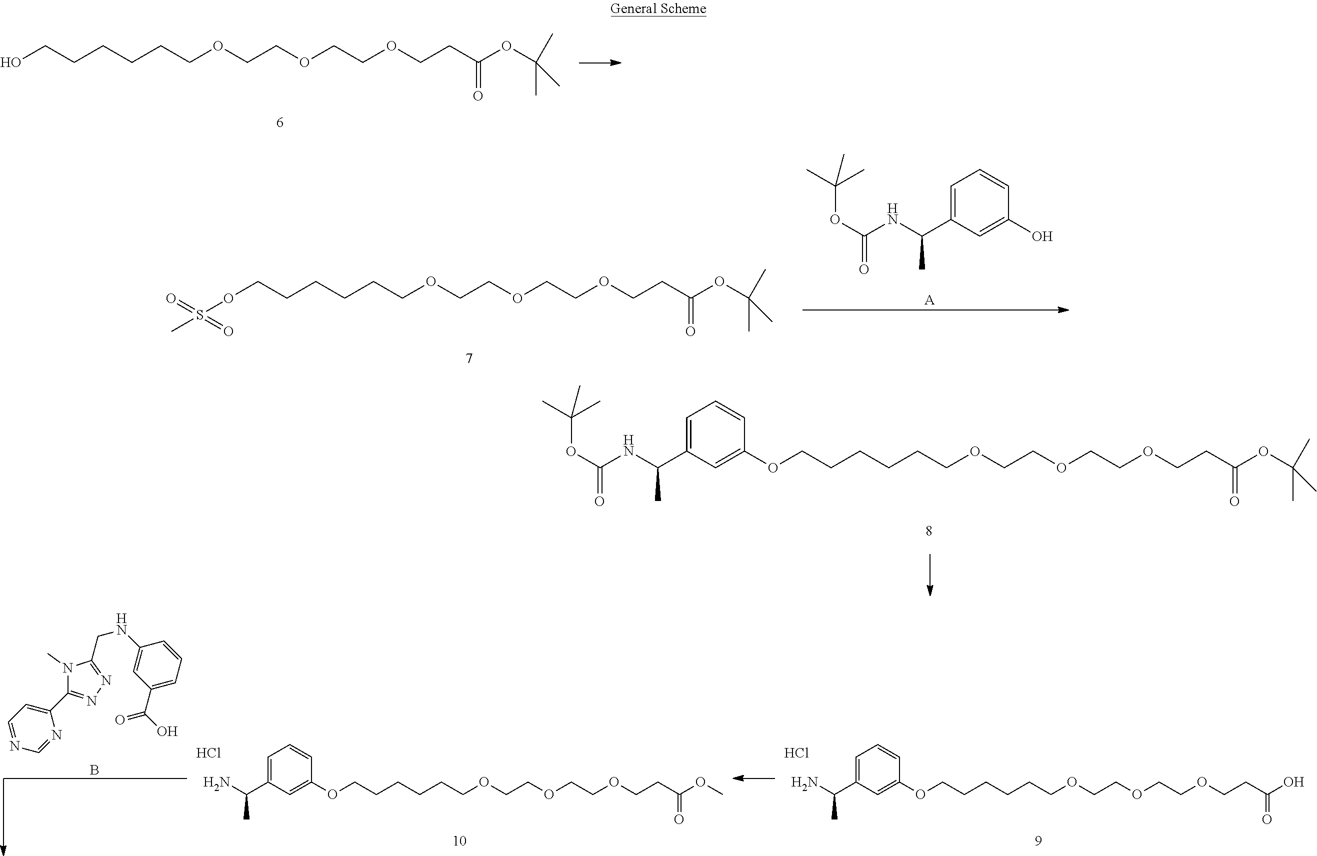

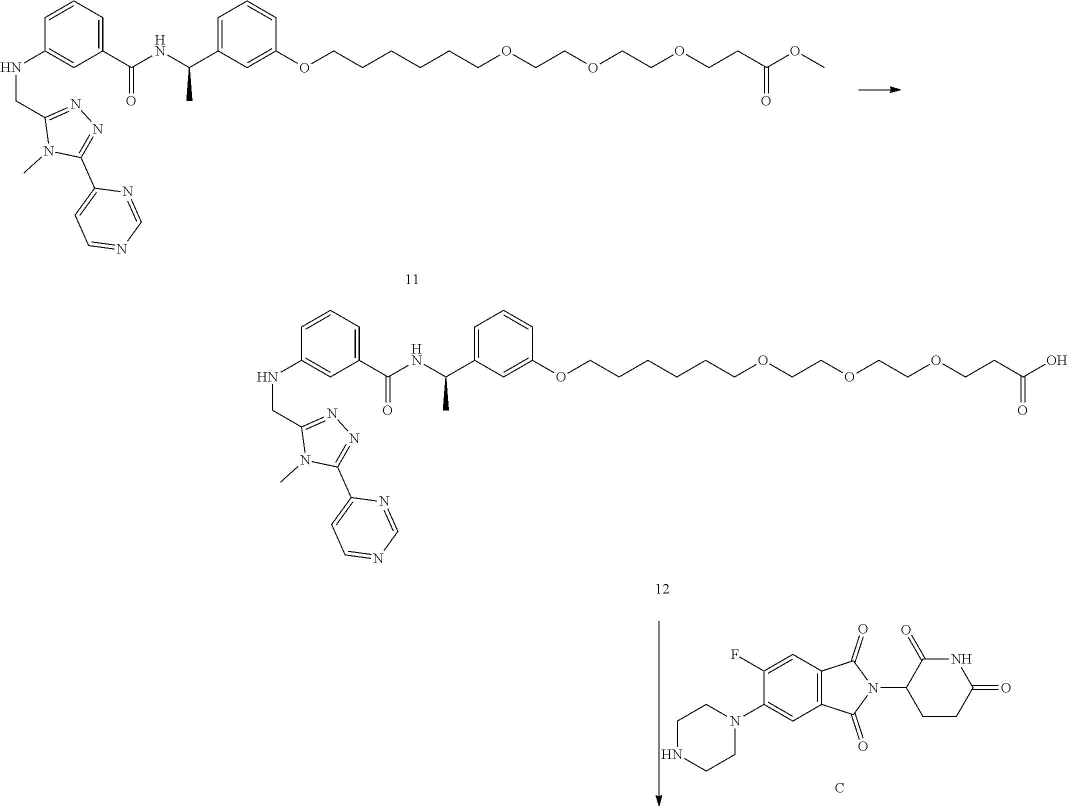

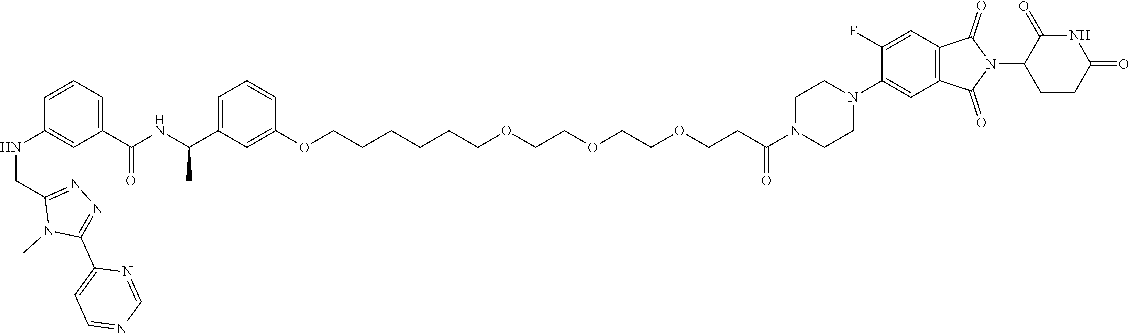 [SMILES: C[C@@H](NC(=O)c1cccc(NCc2nnc(-c3ccncn3)n2C)c1)c1cccc(OCCCCCCOCCOCCOCCC(=O)N2CCN(c3cc4c(cc3F)C(=O)N(C3CCC(=O)NC3=O)C4=O)CC2)c1]

### General Procedures

Compound 7. Solution of compound 6 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 8. A mixture of compound A (2.5 mmol, 1 eq), compound 7 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 8.

Compound 9. A solution of compound 8 (1.2 mmol, 1 eq) in dry DCM (50 mL) with 1.2 ml 3 M HCl in dioxane (5 eq) were stirred for 24 h at r.t. The mixture was evaporated to dryness to give the crude product 12 which was used in the next step without additional purification.

Compound 10. To a solution of compound 9 (1.5 mmol, 1 eq) in dry methanol (50 mL) 0.35 mL of SOCl2 (4.64 mmol) was added. Reaction mixture was stirred for 4 h at r.t. The mixture was evaporated to dryness to give the crude product 10 which was used in the next step without additional purification.

Compound 11. A mixture of compound 10 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq), and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 11.

Compound 12. To a solution of compound 11 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D130. A mixture of compound 12 (0.1 mmol, 1 eq), compound C (or other recruiter) (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was purified by HPLC in acidic conditions (YMC-Pack ODS-AQ 250×20 mml, S-10 μm, 12 nm, gradient water-acetonitrile).

### Synthesis According to the General Procedures

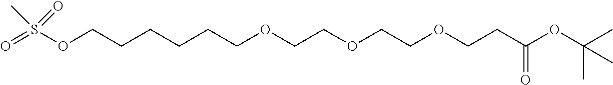 [SMILES: CC(C)(C)OC(=O)CCOCCOCCOCCCCCCOS(C)(=O)=O]

Compound 7. Tert-butyl 3,10,13,16-tetraoxa-2-thianonedecan-19-oate 2,2-dioxide. Yield 97%

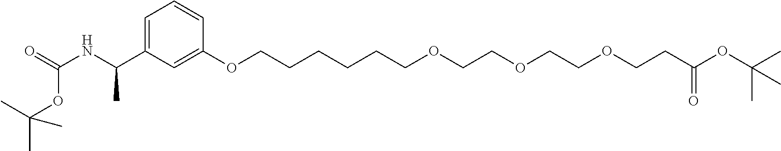 [SMILES: C[C@@H](NC(=O)OC(C)(C)C)c1cccc(OCCCCCCOCCOCCOCCC(=O)OC(C)(C)C)c1]

Compound 8. (R)-tert-butyl 3-(2-(2-(6-(3-(1-(tert-butoxycarbonylamino)ethyl)phenoxy)hexyloxy) ethoxy)ethoxy)propanoate. Colorless oil 63%. LCMS (C18 column 20×2 mm, 2.5 μm, pore size 100 Å, water-acetonitrile+0.1% TFA, gradient 5 to 87% for 3 min, retention time 2.12 min). MS (ESI) m/z 554.6 [MH]+. ¹H NMR (400 MHz, DMSO) δ 7.27 (d, J=8.9 Hz, 1H), 7.18 (t, J=8.6 Hz, 1H), 6.85-6.82 (m, 2H), 6.4 J (dd, J=7.4, 1.5 Hz, 1H), 4.56 (m 1H), 3.91 (t, J=6.4 Hz, 2H), 3.58 (t, J=6.4 Hz, 2H), 3.49-3.45 (m, 8H), 3.37 (t, J=7.4 Hz, 2H), 2.40 (t, J=7.4 Hz, 2H), 1.71-1.65 (m, 2H), 1.52-1.47 (m, 2H), 1.47-1.25 (m, 25H).

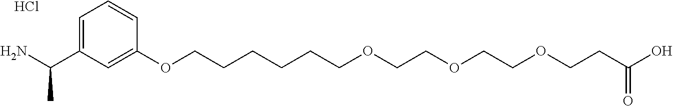 [SMILES: Cl ; C[C@@H](N)c1cccc(OCCCCCCOCCOCCOCCC(=O)O)c1]

Compound 9. ((R)-3-(2-(2-(6-(3-(1-aminoethyl)phenoxy)hexyloxy)ethoxy)ethoxy)propanoic acid hydrochloride. Yield 99%. LCMS (C18 column 20×2 mm, 2.5 μm, pore size 100 Å, water-acetonitrile+0.1% TFA, gradient 5 to 87% for 3 min, retention time 0.99 min). MS (ESI) m/z 398.4 [MH]+.

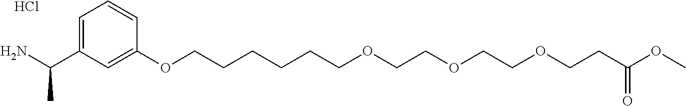 [SMILES: Cl ; COC(=O)CCOCCOCCOCCCCCCOc1cccc([C@@H](C)N)c1]

Compound 10. (R)-methyl 3-(2-(2-(6-(3-(1-aminoethyl)phenoxy)hexyloxy)ethoxy)ethoxy)propanoate hydrochloride. Yield 99%. LCMS (C18 column 20×2 mm, 2.5 μm, pore size 100 Å, water-acetonitrile+0.1% TFA, gradient 5 to 87% for 3 min, retention time 1.17 min). MS (ESI) m/z 412.6[MH]+.

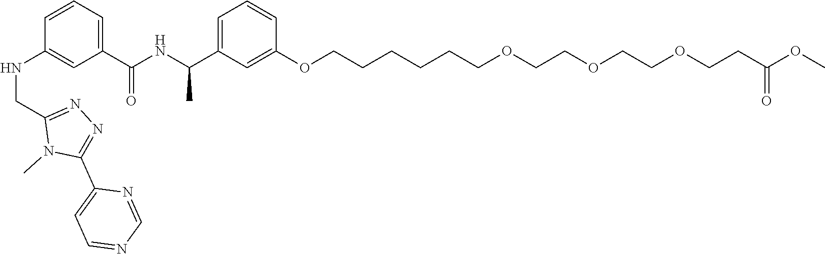 [SMILES: COC(=O)CCOCCOCCOCCCCCCOc1cccc([C@@H](C)NC(=O)c2cccc(NCc3nnc(-c4ccncn4)n3C)c2)c1]

Compound 11. (R)-methyl 3-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)propanoate. Yield 72%. LCMS (C18 column 20×2 mm, 2.5 μm, pore size 100 Å, water-acetonitrile+0.1% TFA, gradient 5 to 87% for 3 min, retention time 1.49 min). MS (ESI) m/z 704.7[MH]+.

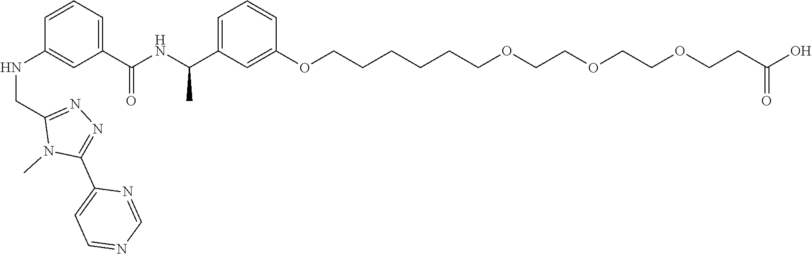 [SMILES: C[C@@H](NC(=O)c1cccc(NCc2nnc(-c3ccncn3)n2C)c1)c1cccc(OCCCCCCOCCOCCOCCC(=O)O)c1]

Compound 12. (R)-3-(2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)ethoxy)propanoic acid. Yield 96%.

LCMS (C18 column 20×2 mm, 2.5 μm, pore size 100 Å, water-acetonitrile+0.1% TFA, gradient 5 to 87% for 3 min, retention time 1.36 min). MS (ESI) m/z 690.5[MH]+.

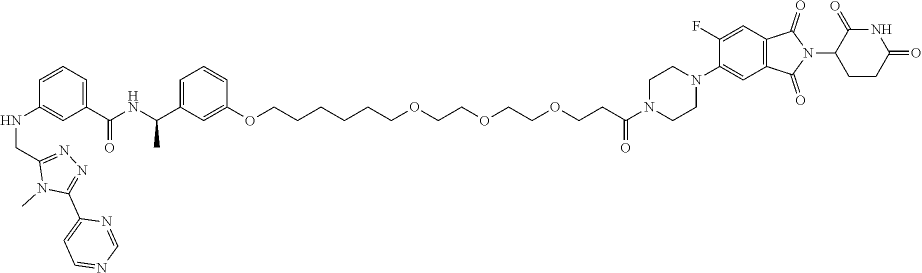 [SMILES: C[C@@H](NC(=O)c1cccc(NCc2nnc(-c3ccncn3)n2C)c1)c1cccc(OCCCCCCOCCOCCOCCC(=O)N2CCN(c3cc4c(cc3F)C(=O)N(C3CCC(=O)NC3=O)C4=O)CC2)c1]

D130. N-((1R)-1-(3-(6-(2-(2-(3-(4-(2-(2,6-dioxopiperidin-3-yl)-6-fluoro-1,3-dioxoisoindolin-5-yl)piperazin-1-yl)-3-oxopropoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 35%. LCMS (C18 column 100×4.6 mm, 5.0 μm, pore size 100 Å, water-acetonitrile+0.1% TFA, gradient 5 to 87% for 10 min, retention time 6.25 min). MS (ESI) m/z 1032.7[MH]+. ¹H NMR (400 MHz, DMSO-d₆) δ 11.08 (s, 1H), 9.33 (s, 1H), 8.96 (d, J=5.3 Hz, 1H), 8.52 (d, J=8.3 Hz, 1H), 8.18 (dd, J=5.3, 1.3 Hz, 1H), 7.72 (d, J=11.3 Hz, 1H), 7.47 (d, J=7.4 Hz, 1H), 7.21-7.15 (m, 3H), 7.11-7.09 (m, 1H), 6.93-6.89 (m, 3H), 6.77 (d, J=8.6 Hz, 1H), 6.44 (t, J=5.7 Hz, 1H), 5.13-5.07 (m, 2H), 4.57 (d, J=5.6 Hz, 2H), 4.07 (s, 3H), 3.90 (t, J=6.5 Hz, 2H), 3.66-3.63 (m, 6H), 3.50-3.43 (m, 9H), 3.35 (t, J=6.5 Hz, 2H), 3.25 (br, 1H), 3.20 (br, 1H), 2.93-2.84 (m, 1H), 2.67-2.54 (m, 4H), 2.07-2.00 (m, 1H), 1.70-1.63 (m, 2H), 1.52-1.30 (m, 10H).

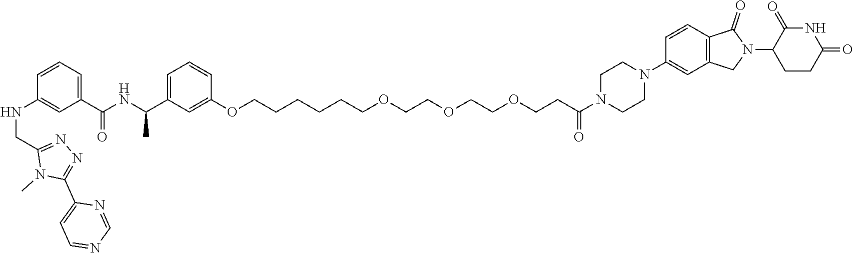 [SMILES: C[C@@H](NC(=O)c1cccc(NCc2nnc(-c3ccncn3)n2C)c1)c1cccc(OCCCCCCOCCOCCOCCC(=O)N2CCN(c3ccc4c(c3)CN(C3CCC(=O)NC3=O)C4=O)CC2)c1]

D131. N-((1R)-1-(3-(6-(2-(2-(3-(4-(2-(2,6-dioxopiperidin-3-yl)-1-oxoisoindolin-5-yl)piperazin-1-yl)-3-oxopropoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 27%. LCMS (C18 column 100×4.6 mm, 5.0 μm, pore size 100 Å, water-acetonitrile+0.1% TFA, gradient 5 to 87% for 10 min, retention time 6.25 min). MS (ESI) m/z 1032.7[MH]+. ¹H NMR (400 MHz, DMSO-d₆) δ 10.91 (s, 1H), 9.33 (s, 1H), 8.97 (d, J=5.3 Hz, 1H), 8.54 (d, J=8.3 Hz, 1H), 8.18 (dd, J=5.3, 1.3 Hz, 1H), 7.54 (d, J=7.4 Hz, 1H), 7.22-7.16 (m, 3H) 7.11-7.04 (m, 2H), 6.93-6.88 (m, 3H), 6.77-6.74 (m, 3H), 6.75 (dd, J=8.1, 1.5 Hz, 1H) 6.44 (t, J=5.7 Hz, 1H), 5.10-5.02 (m, 2H), 4.56 (d, J=5.6 Hz, 2H), 4.35-4.16 (m, 2H), 4.06 (s, 3H), 3.90 (t, J=6.5 Hz, 2H), 3.66-3.60 (m, 6H), 3.49-3.41 (m, 9H), 3.35 (t, J=6.5 Hz, 2H), 3.27-3.25 (br, 2H), 2.94-2.86 (m, 1H), 2.67-2.54 (m, 4H), 2.41-2.30 (m, 1H), 1.99-1.92 (m, 1H), 1.69-1.63 (m, 2H), 1.52-1.30 (m, 9H).

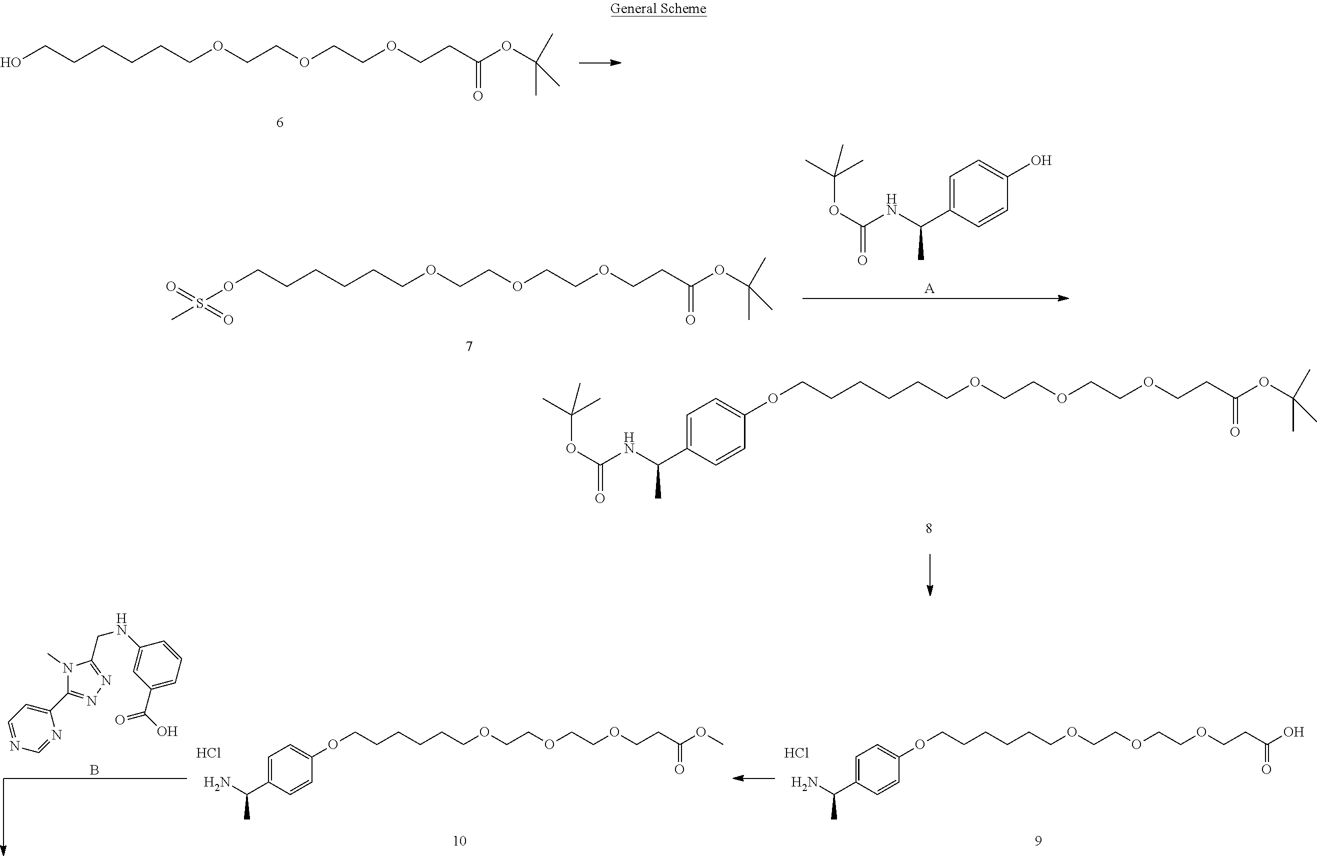

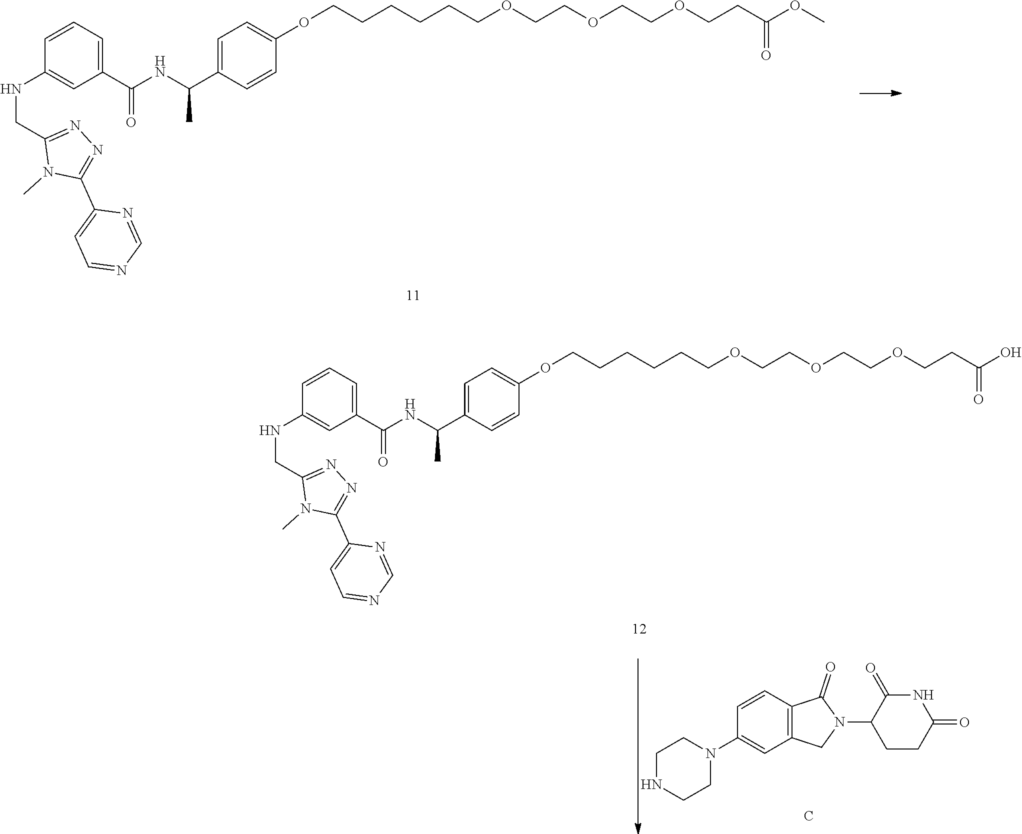

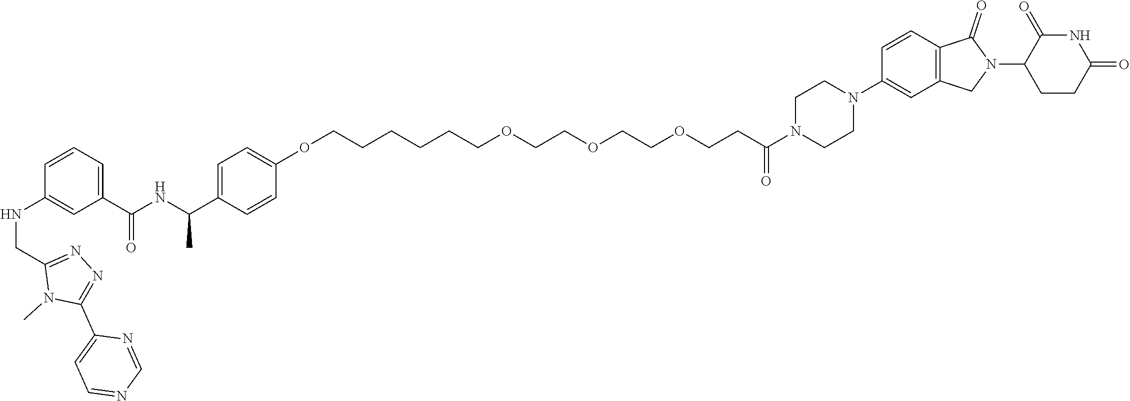 [SMILES: C[C@@H](NC(=O)c1cccc(NCc2nnc(-c3ccncn3)n2C)c1)c1ccc(OCCCCCCOCCOCCOCCC(=O)N2CCN(c3ccc4c(c3)CN(C3CCC(=O)NC3=O)C4=O)CC2)cc1]

### General Procedures

Compound 7. Solution of compound 6 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 8. A mixture of compound A (2.5 mmol, 1 eq), compound 7 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 8.

Compound 9. A solution of compound 8 (1.2 mmol, 1 eq) in dry DCM (50 mL) with 1.2 ml 3 M HCl in dioxane (5 eq) were stirred for 24 h at r.t. The mixture was evaporated to dryness to give the crude product 12 which was used in the next step without additional purification.

Compound 10. To a solution of compound 9 (1.5 mmol, 1 eq) in dry methanol (50 mL) 0.35 mL of SOCl2 (4.64 mmol) was added. Reaction mixture was stirred for 4 h at r.t. The mixture was evaporated to dryness to give the crude product 10 which was used in the next step without additional purification.

Compound 11. A mixture of compound 10 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq) and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 11.

Compound 12. To a solution of compound 11 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D136. A mixture of compound 12 (0.1 mmol, 1 eq), compound C (or other recruiter) (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was purified by HPLC in acidic conditions (YMC-Pack ODS-AQ 250×20 mml, S-10 μm, 12 nm, gradient water-acetonitrile).

### Synthesis According to the General Procedures

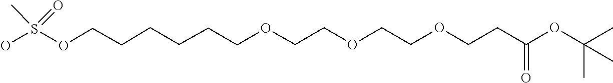

Compound 7. Tert-butyl 3,10,13,16-tetraoxa-2-thianonedecan-19-oate 2,2-dioxide. Yield 97%

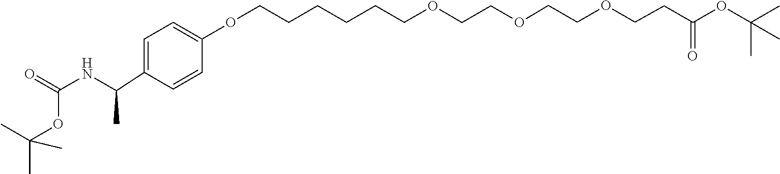 [SMILES: C[C@@H](NC(=O)OC(C)(C)C)c1ccc(OCCCCCCOCCOCCOCCC(=O)OC(C)(C)C)cc1]

Compound 8. (R)-tert-butyl 3-(2-(2-(6-(4-(1-(tert-butoxycarbonylamino)ethyl)phenoxy) hexyloxy)ethoxy)ethoxy)propanoate. Colorless oil 55%. LCMS (C18 column 20×2 mm, 2.5 μm, pore size 100 Å, water-acetonitrile+0.1% TFA, gradient 5 to 87% for 3 min, retention time 2.12 min). MS (ESI) m/z 554.5 [MH]+. ¹H NMR (400 MHz, DMSO) δ 7.25 (d, J=8.9 Hz, 1H), 7.18 (d, J=8.6 Hz, 2H), 6.83 (d, J=8.6 Hz, 2H 2H), 4.56 (m 1H), 3.91 (t, J=6.4 Hz, 2H), 3.58 (t, J=6.4 Hz, 2H), 3.49-3.45 (m, 8H), 3.37 (t, J=7.4 Hz, 2H), 2.40 (t, J=7.4 Hz, 2H), 1.71-1.65 (m, 2H), 1.52-1.47 (m, 2H), 1.47-1.25 (m, 25H)

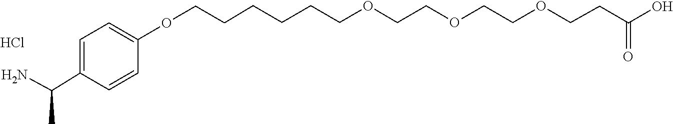 [SMILES: Cl ; C[C@@H](N)c1ccc(OCCCCCCOCCOCCOCCC(=O)O)cc1]

Compound 9. (R)-3-(2-(2-(6-(4-(1-aminoethyl)phenoxy)hexyloxy)ethoxy)ethoxy)propanoic acid hydrochloride. Yield 99%. LCMS (C18 column 20×2 mm, 2.5 μm, pore size 100 Å, water-acetonitrile+0.1% TFA, gradient 5 to 87% for 3 min, retention time 1.07 min). MS (ESI) m/z 398.5 [MH]+.

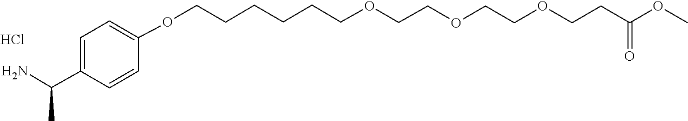 [SMILES: Cl ; COC(=O)CCOCCOCCOCCCCCCOc1ccc([C@@H](C)N)cc1]

Compound 10. (R)-methyl 3-(2-(2-(6-(4-(1-aminoethyl)phenoxy)hexyloxy)ethoxy)ethoxy)propanoate hydrochloride. Yield 99%. LCMS (C18 column 20×2 mm, 2.5 μm, pore size 100 Å, water-acetonitrile+0.1% TFA, gradient 5 to 87% for 3 min, retention time 1.14 min). MS (ESI) m/z 412.8[MH]+.

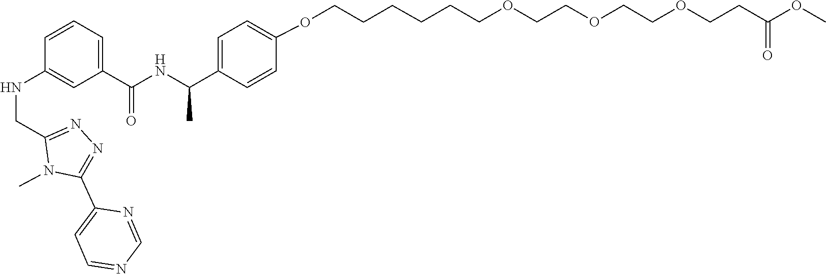 [SMILES: COC(=O)CCOCCOCCOCCCCCCOc1ccc([C@@H](C)NC(=O)c2cccc(NCc3nnc(-c4ccncn4)n3C)c2)cc1]

Compound 11. (R)-methyl 3-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)propanoate. Yield 65%. LCMS (C18 column 20×2 mm, 2.5 μm, pore size 100 Å, water-acetonitrile+0.1% TFA, gradient 5 to 87% for 3 min, retention time 1.49 min). MS (ESI) m/z 704.7[MH]+.

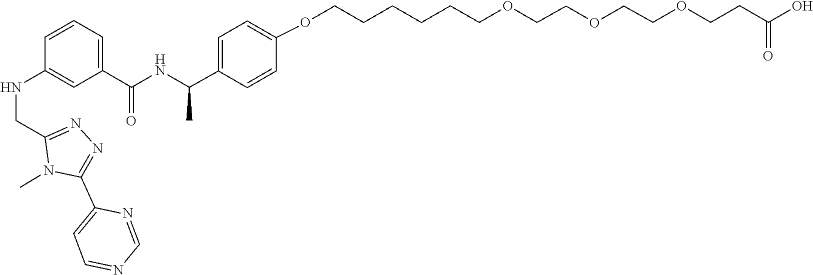 [SMILES: C[C@@H](NC(=O)c1cccc(NCc2nnc(-c3ccncn3)n2C)c1)c1ccc(OCCCCCCOCCOCCOCCC(=O)O)cc1]

Compound 12. (R)-3-(2-(2-(6-(4-(1-(3-((4-methyl-5-(pyrimidin-4-yl)-4H-1,2,4-triazol-3-yl)methylamino)benzamido)ethyl)phenoxy)hexyloxy)ethoxy)ethoxy)propanoic acid. Yield 96%. LCMS (C18 column 20×2 mm, 2.5 μm, pore size 100 Å, water-acetonitrile+0.1% TFA, gradient 5 to 87% for 3 min, retention time 1.36 min). MS (ESI) m/z 690.5[MH]+.

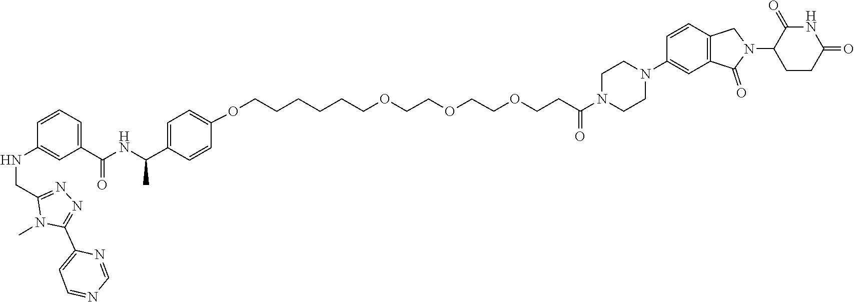 [SMILES: C[C@@H](NC(=O)c1cccc(NCc2nnc(-c3ccncn3)n2C)c1)c1ccc(OCCCCCCOCCOCCOCCC(=O)N2CCN(c3ccc4c(c3)C(=O)N(C3CCC(=O)NC3=O)C4)CC2)cc1]

D136. N-((1R)-1-(4-(6-(2-(2-(3-(4-(2-(2,6-dioxopiperidin-3-yl)-3-oxoisoindolin-5-yl)piperazin-1-yl)-3-oxopropoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 27%. LCMS (C18 column 100×4.6 mm, 5.0 μm, pore size 100 Å, water-acetonitrile+0.1% TFA, gradient 5 to 87% for 10 min, retention time 5.63 min). MS (ESI) m/z 1001.2[MH]+. ¹H NMR (400 MHz, DMSO-d₆) δ 10.93 (s, 1H), 9.33 (s, 1H), 8.97 (d, J=5.3 Hz, 1H), 8.49 (d, J=8.3 Hz, 1H), 8.18 (dd, J=5.3, 1.3 Hz, 1H), 7.52 (d, J=7.4 Hz, 1H), 7.28 (d, J=8.7 Hz, 2H) 7.18-7.14 (m, 2H), 7.09-7.05 (m, 3H), 6.90-6.84 (m, 3H), 6.44 (t, J=5.7 Hz, 1H), 5.10-5.02 (m, 2H), 4.56 (d, J=5.6 Hz, 2H), 4.35-4.16 (m, 2H), 4.06 (s, 3H), 3.90 (t, J=6.5 Hz, 2H), 3.66-3.60 (m, 6H), 3.49-3.41 (m, 9H), 3.35 (t, J=6.5 Hz, 2H), 3.27-3.25 (br, 2H), 2.94-2.86 (m, 1H), 2.67-2.54 (m, 4H), 2.41-2.30 (m, 1H), 1.99-1.92 (m, 1H), 1.69-1.63 (m, 2H), 1.52-1.30 (m, 9H).

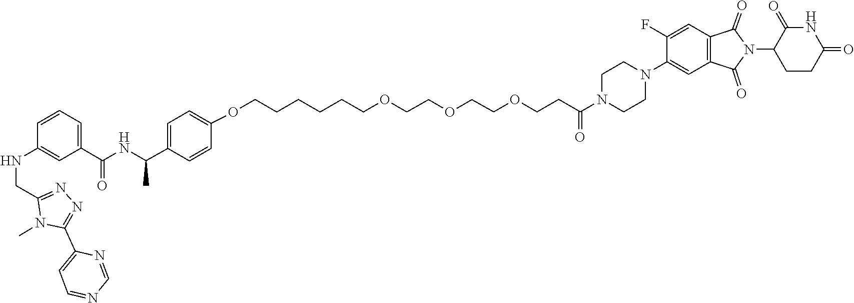 [SMILES: C[C@@H](NC(=O)c1cccc(NCc2nnc(-c3ccncn3)n2C)c1)c1ccc(OCCCCCCOCCOCCOCCC(=O)N2CCN(c3cc4c(cc3F)C(=O)N(C3CCC(=O)NC3=O)C4=O)CC2)cc1]

D137. N-((1R)-1-(4-(6-(2-(2-(3-(4-(2-(2,6-dioxopiperidin-3-yl)-6-fluoro-1,3-dioxoisoindolin-5-yl)piperazin-1-yl)-3-oxopropoxy)ethoxy)ethoxy)hexyloxy)phenyl)ethyl)-3-((4-methyl-5-(pyrimidin-4-yl)-4H-1,2,4-triazol-3-yl)methylamino)benzamide. Yield 31%. LCMS (C18 column 100×4.6 mm, 5.0 μm, pore size 100 Å, water-acetonitrile+0.1% TFA, gradient 5 to 87% for 10 min, retention time 6.23 min). MS (ESI) m/z 1032.7 [MH]+. ¹H NMR (400 MHz, DMSO-d₆) δ 11.10 (s, 1H), 9.33 (s, 1H), 8.96 (d, J=5.3 Hz, 1H), 8.49 (d, J=8.3 Hz, 1H), 8.18 (dd, J=5.3, 1.3 Hz, 1H), 7.76 (d, J=11.3 Hz, 1H), 7.47 (d, J=7.4 Hz, 1H), 7.28 (d, J=8.7 Hz, 2H) 7.18-7.07 (m, 3H), 6.90-6.83 (m, 3H), 6.44 (t, J=5.7 Hz, 1H), 5.13-5.07 (m, 2H), 4.57 (d, J=5.6 Hz, 2H), 4.07 (s, 3H), 3.90 (t, J=6.5 Hz, 2H), 3.66-3.63 (m, 6H), 3.50-3.43 (m, 9H), 3.35 (t, J=6.5 Hz, 2H), 3.25 (br, 1H), 3.20 (br, 1H), 2.93-2.84 (m, 1H), 2.67-2.54 (m, 4H), 2.07-2.00 (m, 1H), 1.70-1.63 (m, 2H), 1.52-1.30 (m, 10H).

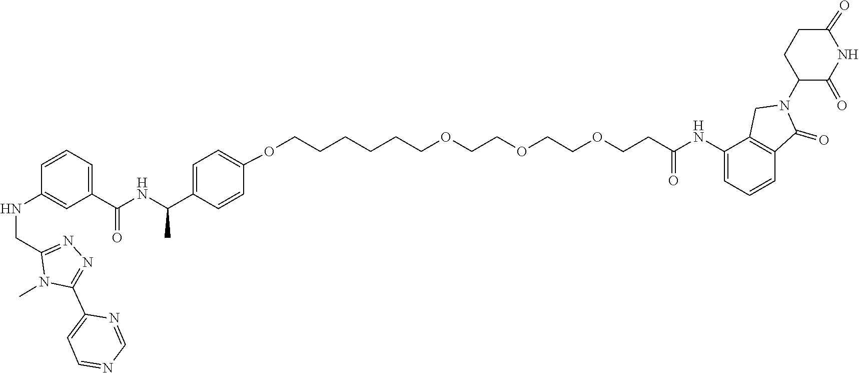 [SMILES: C[C@@H](NC(=O)c1cccc(NCc2nnc(-c3ccncn3)n2C)c1)c1ccc(OCCCCCCOCCOCCOCCC(=O)Nc2cccc3c2CN(C2CCC(=O)NC2=O)C3=O)cc1]

D142. N-((1R)-1-(4-(6-(2-(2-(3-(2-(2,6-dioxopiperidin-3-yl)-1-oxoisoindolin-4-ylamino)-3-oxopropoxy)ethoxy)ethoxy)hexyloxy)phenyl)ethyl)-3-((4-methyl-5-(pyrimidin-4-yl)-4H-1,2,4-triazol-3-yl)methylamino)benzamide. LCMS (C18 column 100×4.6 mm, 5.0 μm, pore size 100 Å, water-acetonitrile+0.1% TFA, gradient 5 to 87% for 10 min, retention time 5.71 min). MS (ESI) m/z 931.7[MH]+. ¹H NMR (400 MHz, DMSO-d₆) δ 11.00 (s, 1H), 9.81 (s, 1H), 9.33 (s, 1H), 8.97 (d, J=5.3 Hz, 1H), 8.52 (d, J=8.3 Hz, 1H), 8.17 (dd, J=5.3, 1.3 Hz, 1H), 7.81 (d, J=11.3 Hz, 1H), 7.53-7.46 (m, 2H), 7.26 (d, J=8.7 Hz, 2H) 7.18-7.15 (m, 2H), 7.09-7.07 (m, 1H), 6.90-6.84 (m, 3H), 6.44 (t, J=5.7 Hz, 1H), 5.16-5.07 (m, 2H), 4.58 (d, J=5.6 Hz, 2H), 4.41-4.30 (m, 2H), 4.07 (s, 3H), 3.90 (t, J=6.5 Hz, 2H), 3.71 (t, J=6.5 Hz, 2H), 3.52-3.47 (m, 6H), 3.43-3.40 (m, 2H), 3.33 (t, J=6.5 Hz, 2H), 2.96-2.88 (m, 1H), 2.67-2.54 (m, 3H), 2.39-2.28 (m, 1H), 2.06-1.99 (m, 1H), 1.69-1.63 (m, 2H), 1.50-1.29 (m, 9H).

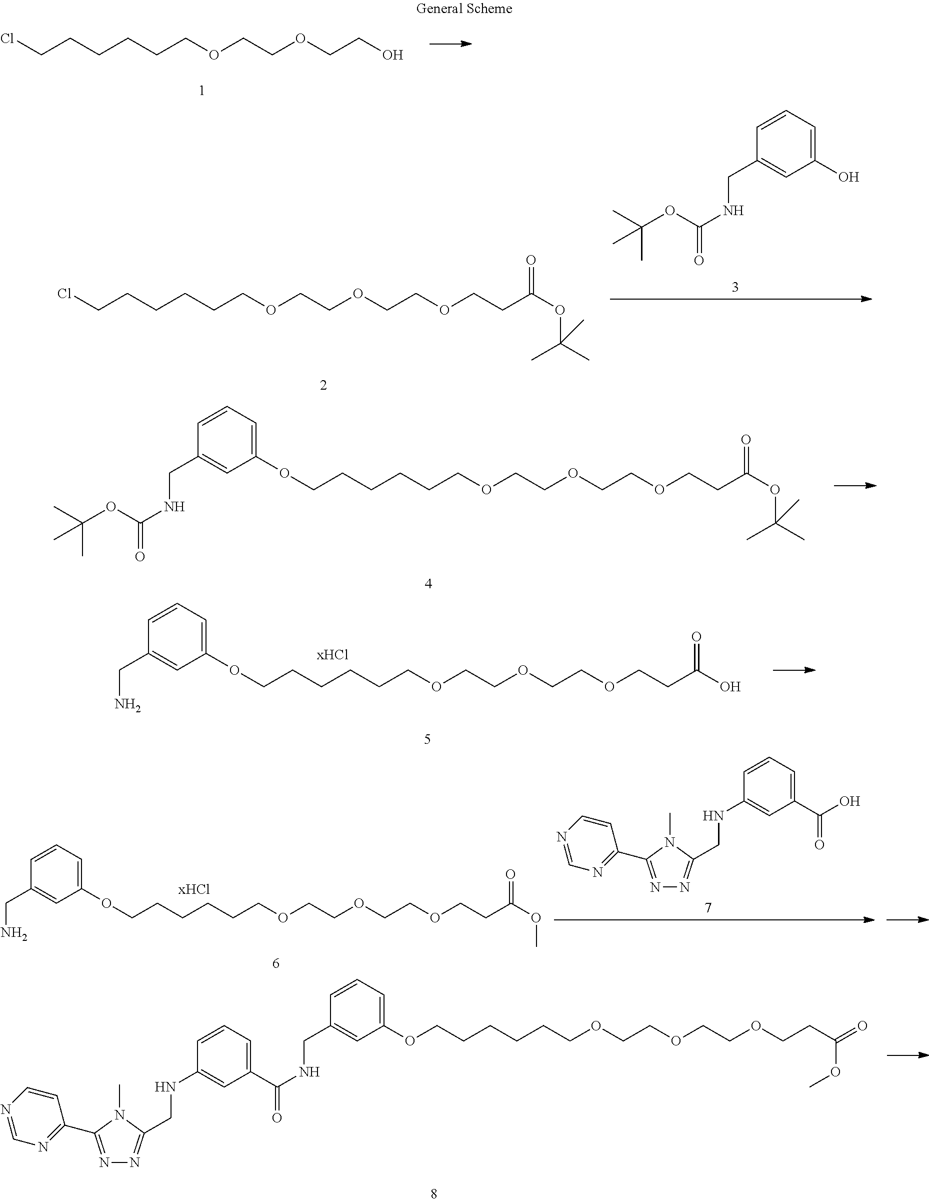

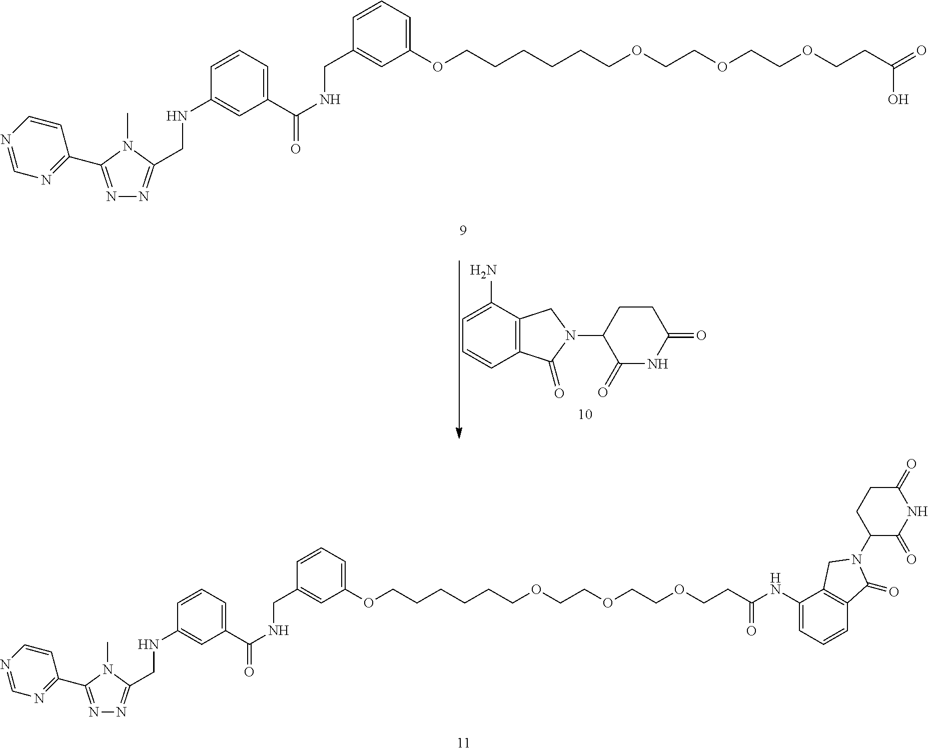 [SMILES: Cn1c(CNc2cccc(C(=O)NCc3cccc(OCCCCCCOCCOCCOCCC(=O)O)c3)c2)nnc1-c1ccncn1 ; Nc1cccc2c1CN(C1CCC(=O)NC1=O)C2=O ; Cn1c(CNc2cccc(C(=O)NCc3cccc(OCCCCCCOCCOCCOCCC(=O)Nc4cccc5c4CN(C4CCC(=O)NC4=O)C5=O)c3)c2)nnc1-c1ccncn1]

### General Procedures

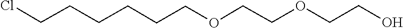 [SMILES: OCCOCCOCCCCCCCl]

### Compound 1. 2-(2-((6-Chlorohexyl)oxy)ethoxy)ethanol

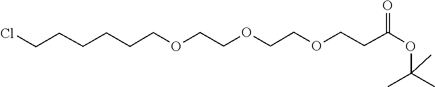 [SMILES: CC(C)(C)OC(=O)CCOCCOCCOCCCCCCCl]

### Compound 2. tert-butyl 3-(2-(2-((6-chlorohexyl)oxy)ethoxy)ethoxy)propanoate

To a solution of compound 1 (5.6 g, 24.9 mmol) and t-butylacrylate (4.8 g, 37.4 mmol) in anhydrous THF (50 ml) catalytic amount of NaH was added then the reaction mixture was stirred at rt for 16 h. The reaction mixture was evaporated. The residue was purified by column chromatography, eluting with hexane-ether 5:1 to 3:1 to give product as colorless oil. Yield 6.4 g, 74%. ¹H NMR (400 MHz, DMSO-d₆) δ 3.60 (dt, J=12.8, 6.4 Hz, 4H), 3.54-3.41 (m, 8H), 3.36 (t, J=6.5 Hz, 2H), 2.40 (t, J=6.2 Hz, 2H), 1.77-1.64 (m, 2H), 1.57-1.43 (m, 2H), 1.43-1.35 (m, 11H), 1.35-1.25 (m, 2H).

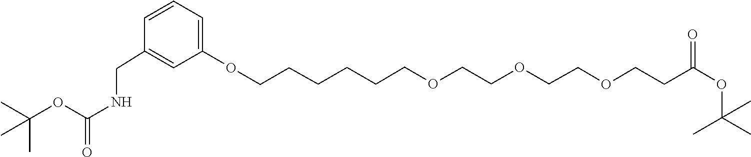 [SMILES: CC(C)(C)OC(=O)CCOCCOCCOCCCCCCOc1cccc(CNC(=O)OC(C)(C)C)c1]

Compound 4. tert-butyl 3-(2-(2-((6-(3-(((Tert-butoxycarbonyl)amino)methyl)phenoxy) hexyl)oxy)ethoxy)ethoxy)propanoate. Compound 2 (0.95 g, 2.7 mmol), compound 3 (0.6 g, 2.7 mmol, 1 eq), K₂CO₃ (0.74 g, 5.4 mmol, 2 eq) and KI (89 mg, 0.54 mmol, 0.2 eq) in dry acetonitrile (30 ml) were stirred for 72 h at reflux. The reaction mixture was evaporated, the residue was suspended in methylene chloride, washed with water and purified by column chromatography using hexane-EtOAc-Et3N 66:33:5 to give product as colorless oil. Yield 1.3 g, 90%. ¹H NMR (400 MHz, DMSO-d₆) δ 7.33 (s, J=5.1 Hz, 1H), 7.19 (t, J=7.9 Hz, 1H), 6.85-6.69 (m, 3H), 4.06 (d, J=9.9 Hz, 2H), 3.92 (t, J=6.4 Hz, 2H), 3.62-3.53 (m, 2H), 3.53-3.42 (m, 8H), 3.37 (t, J=6.5 Hz, 2H), 2.40 (t, J=6.2 Hz, 2H), 1.75-1.64 (m, 2H), 1.57-1.46 (m, 2H), 1.46-1.27 (m, 22H).

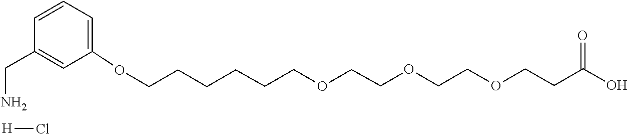

### Compound 5. 3-(2-(2-((6-(3-(Aminomethyl)phenoxy)hexyl)oxy)ethoxy)ethoxy)propanoic acid hydrochloride

A solution of compound 4 (1.3 g, 2.4 mmol) in dry DCM (20 ml) with 4.0 ml 3 M HCl in dioxane (5 eq) were stirred for 24 h at rt. The mixture was evaporated to dryness to give the crude product 5 which was used in the next step without additional purification.

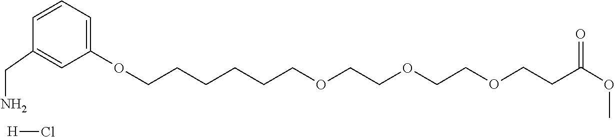

Compound 6. Methyl 3-(2-(2-((6-(3-(aminomethyl)phenoxy)hexyl)oxy)ethoxy)ethoxy)propanoate hydrochloride. To a solution of compound 5 (1.0 g, 2.4 mmol) in dry MeOH (30 ml) was added dropwise SOCl₂ (0.36 ml, 4.8 mmol, 2 eq). The mixture was stirred for 12 h at r.t. and then evaporated to dryness to give the crude product 5 which no need additional purification. Yield 1.0 g, 100%. ¹H NMR (400 MHz, DMSO-d₆) δ 8.37 (s, 3H), 7.30 (t, J=7.9 Hz, 1H), 7.10 (s, 1H), 7.01 (d, J=7.4 Hz, 1H), 6.92 (d, J=6.3 Hz, 1H), 4.04-3.88 (m, 4H), 3.66-3.55 (m, 5H), 3.53-3.42 (m, 8H), 3.41-3.35 (m, 2H), 2.53 (d, J=10.3 Hz, 2H), 1.78-1.65 (m, 2H), 1.57-1.46 (m, 2H), 1.46-1.28 (m, 4H).

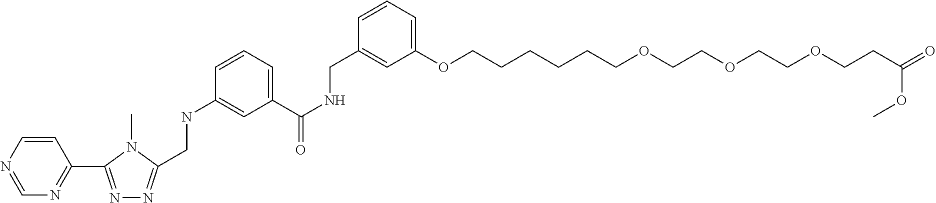 [SMILES: COC(=O)CCOCCOCCOCCCCCCOc1cccc(CNC(=O)c2cccc(NCc3nnc(-c4ccncn4)n3C)c2)c1]

Compound 8. Methyl 3-(2-(2-((6-(3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)propanoate. A mixture of compound 6 (250 mg, 0.57 mmol), compound 7 (178 mg, 0.7 mmol, 1 eq) and TBTU (221 mg, 0.68 mmol, 1.2 eq) in pyridine (10 ml) were stirred at rt for 24 h. The mixture was evaporated to dryness. The residue was dissolved in methylene chloride, washed with water then purified by column chromatography eluting with EtOAc-MeOH 10:1 to give product as colorless oil. Yield 130 mg, 33%. LCMS (C18 column 100×4.6 mm, 5.0 μm, pore size 100 Å, water-acetonitrile+0.1% TFA, gradient 5 to 87% for 3 min, retention time 1.44 min). MS (ESI) m/z 690.5 [MH]+.

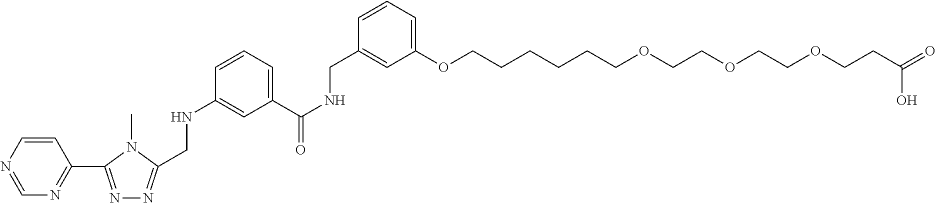 [SMILES: Cn1c(CNc2cccc(C(=O)NCc3cccc(OCCCCCCOCCOCCOCCC(=O)O)c3)c2)nnc1-c1ccncn1]

Compound 9. 3-(2-(2-((6-(3-((3-(((4-methyl-5-(pyrimidin-4-yl)-4H-1,2,4-triazol-3-yl)methyl)amino)benzamido)methyl)phenoxy)hexyl)oxy)ethoxy)ethoxy)propanoic acid To a solution of compound 8 (130 mg, 0.19 mmol) in MeOH (20 mL) was added a solution KOH (21 mg, 3.8 mmol, 2 eq) in 3 ml H₂O. The mixture was stirred for 16 h at r.t. and then MeOH evaporated. To the residue was added 15 ml of H₂O and the mixture was acidified to pH=5 with 1N aq. HCl and aqueous phase was extracted with 4*15 ml of DCM. Combined organic phases were washed with brine, dried over Na₂SO₄, and evaporated. The product was used in the next step without additional purification. Yield 110 mg, 87%. LCMS (C18 column 100×4.6 mm, 5.0 μm, pore size 100 Å, water-acetonitrile+0.1% TFA, gradient 5 to 87% for 3 min, retention time 1.31 min). MS (ESI) m/z 676.5 [MH]+.

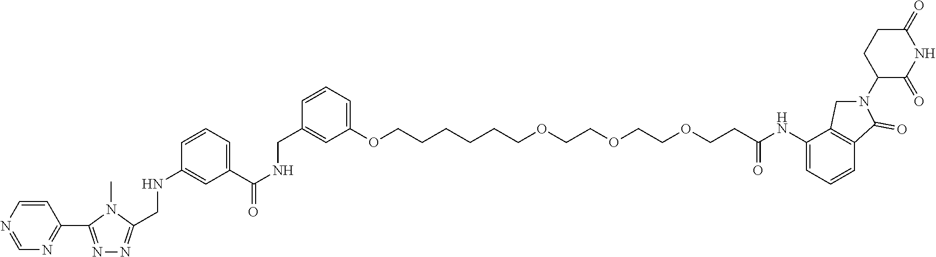 [SMILES: Cn1c(CNc2cccc(C(=O)NCc3cccc(OCCCCCCOCCOCCOCCC(=O)Nc4cccc5c4CN(C4CCC(=O)NC4=O)C5=O)c3)c2)nnc1-c1ccncn1]

Compound 11. D195. N-(3-(6-(2-(2-(3-(2-(2,6-dioxopiperidin-3-yl)-1-oxoisoindolin-4-ylamino)-3-oxopropoxy)ethoxy)ethoxy)hexyloxy)benzyl)-3-((4-methyl-5-(pyrimidin-4-yl)-4H-1,2,4-triazol-3-yl)methylamino)benzamide. A mixture of compound 9 (110 mg, 0.13 mmol), compound 10 (70 mg, 0.13 mmol, 1 eq) and TBTU (79 mg, 0.16 mmol, 1.2 eq) in pyridine (10 ml) were stirred at rt for 24 h. The mixture was evaporated to dryness. The residue was purified by HPLC to give product as a white powder. Yield 32 mg, 18%. LCMS (C18 column 100×4.6 mm, 5.0 μm, pore size 100 Å, water-acetonitrile+0.1% TFA, gradient 5 to 87% for 10 min, retention time 6.40 min). MS (ESI) m/z 1088.7 [MH]+. ¹H NMR (400 MHz, DMSO-d₆): δ 11.01 (s, 1H), 9.81 (s, 1H), 9.33 (s, 1H), 8.96 (d, 1H), 8.81 (t, J=5.2 Hz, 1H), 8.18 (d, J=5.2 Hz, 1H), 7.80 (d, J=9.9 Hz, 1H), 7.51-7.46 (m, 2H), 7.24-7.05 (m, 4H), 6.92-6.76 (m, 4H), 6.47 (t, J=5.5 Hz, 1H), 5.17 (dd, J=14.5, 6.2 Hz, 1H), 4.58 (d, 2H), 4.41-4.33 (m, 4H), 4.07 (s, 3H), 3.90 (t, J=6.4 Hz, 2H), 3.71 (t, J=6.4 Hz, 2H), 3.51-3.40 (m, 9H), 2.96-2.87 (m, 1H), 2.61-2.58 (m, 4H), 2.37-2.26 (m, 1H), 2.04-1.99 (m, 1H), 1.69-1.63 (m, 2H), 1.49-1.27 (m, 6H).

### Example 25: Preparation of D135, D138, D139, D140, D141, D142, D170, and D193

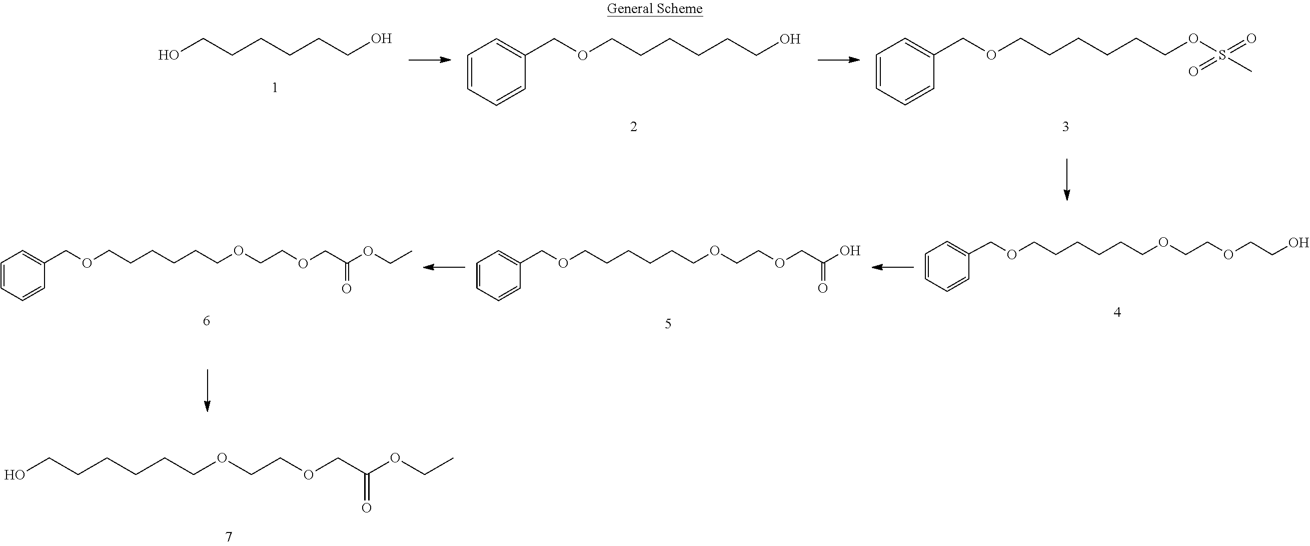

### General Procedures

Compound 2. A mixture of compounds 1 (65.8 g, 631 mmol, 4 eq), benzyl chloride (20.0 g, 158 mmol, 1 eq.), TBAB (0.2 g) and 50% aqua solution of NaOH (25.3 g, 631 mmol, 1 eq) was stirred at 100° C. for 12 h before been poured in water (200 ml). The product was extracted with Et₂O (3×150 ml), the organic layers were dried over Na₂SO₄, the solvent was removed under reduced pressure. The residue was purified by column chromatography on silica gel, using hexane/EtOAc=1:1 as eluent, to provide the product 2. Yield 85%. ¹H NMR (400 MHz, DMSO-d₆) δ 7.36-7.27 (m, 5H), 4.43 (s, 2H), 4.33 (t, J=4.3 Hz, 1H), 3.42-3.37 (m, 4H), 1.56-1.49 (m, 2H), 1.44-1.37 (m, 2H), 1.35-1.25 (m, 4H).

Compound 3. A solution of compound 2 (15.8 g, 75.85 mmol) in CH₂Cl₂ was mixed with 19.8 mL of DIPEA (113.77 mmol) and cooled to 0° C. 10.42 g of MsCl was added dropwise. The mixture was stirred at room temperature for 2 hours, washed with water, dried over Na₂SO₄ and evaporated. The product was used without purification. Yield 21.9 g (98%). ¹H NMR (400 MHz, DMSO-d₆) δ 7.36-7.27 (m, 5H), 4.44 (s, 2H), 4.19 (t, J=6.2 Hz, 2H), 3.42 (t, J=6.4 Hz, 2H), 3.15 (s, 3H), 1.69-1.62 (m, 2H), 1.56-1.51 (m, 2H), 1.41-1.30 (m, 4H).

Compound 4. An oven-dried 500 mL flask with stirrer was charged with sodium hydride (60% dispersion in mineral oil, 7.5 g, 187 mmol). The flask was sealed with a septum, evacuated and back-filled with argon (3 times). Anhydrous DMF (100 mL) followed by diethylene glycol (24.1 g, 227.5 mmol) were added and the reaction mixture stirred at 25° C. for 2 hr. A solution of 5-benzyloxyhexthyl 4-methanesulfonate 3 (21.7 g, 75.8 mmol) in anhydrous DMF (200 mL) was added to the mixture. The solution was warmed to 50° C. and stirred for 18 hr before been cooled to r.t. and concentrated in vacuo. The residue was partitioned between H₂O (400 mL) and Et₂O (500 mL). The organic layer was dried over Na₂SO₄ and concentrated in vacuo. The crude yellow oil was then purified by flash chromatography (200 g SiO₂, EtOAc and hexane (50/50)) providing compound 4 (8.5 g, 38% yield) as viscous oil. ¹H NMR (400 MHz, DMSO-d₆) δ 7.36-7.27 (m, 5H), 4.55 (t, J=5.5 Hz, 1H), 4.44 (s, 2H), 3.51-3.45 (m, 6H), 3.42-3.39 (m, 4H), 3.36 (t, J=6.6 Hz, 2H), 1.55-1.46 (m, 4H), 1.33-1.27 (m, 4H).

Compound 5. Jones reagent (prepared from 19.5 g (194 mmol) of CrO₃, 405 mL of water and 39 mL of conc.H₂SO₄) was added to the solution of compound 4 (19.2 g, 28 mmol) in acetone (600 mL) at 0° C. Reaction mixture was stirred at room temperature overnight; excess of oxidative reagent was neutralized with i-PrOH (50 mL). A solution was concentrated, diluted with water (1000 mL) and extracted with CH₂Cl₂ (3×500 mL). Organic phase was dried over Na₂SO₄, solvent was removed in vacuo. Compound 5 was used without purification. Yield 19.6 g (97%). ¹H NMR (400 MHz, DMSO-d₆) δ 12.55 (br, 1H), 7.36-7.25 (m, 5H), 4.44 (s, 2H), 4.01 (s, 2H), 3.58-3.56 (m, 2H), 3.49-3.46 (m, 2H), 3.41 (t, 2H), 3.36 (t, 2H), 1.56-1.43 (m, 4H), 1.35-1.25 (m, 4H).

Compound 6. 9 mL (126 mmol) of SOCl₂ was added to the stirred solution of compound 5 (19.6 g, 63 mmol) in dry ethanol (300 mL) at room temperature. After 8 hours reaction mixture was concentrated and purified with column chromatography (SiO₂, ethyl acetate (33%)-hexane (66%)). Yield 13.6 g (63%). ¹H NMR (400 MHz, DMSO-d₆) δ 7.36-7.25 (m, 5H), 4.44 (s, 2H), 4.13-4.03 (m, 4H), 3.59-3.57 (m, 2H), 3.49-3.47 (m, 2H), 3.41 (t, J=6.4 Hz, 2H), 3.36 (t, J=6.4 Hz, 2H), 1.56-1.44 (m, 4H), 1.33-1.27 (m, 4H), 1.19 (t, J=7.1 Hz, 3H).

Compound 7. H₂ gas was passed through a mixture of compound 6 (13.6 g, 40 mmol) and 10% Pd/C (4.3 g) in EtOH (50 ml) at r.t. for 12 h. The powder of Pd/C was filtered, and the solvent was removed under reduced pressure to provide the product 7. Yield: 8.6 g (97%).

¹H NMR (400 MHz, DMSO-d₆) δ 4.31 (t, J=5.1 Hz, 1H), 4.14-4.10 (m, 4H), 3.59-3.57 (m, 2H), 3.49-3.47 (m, 2H), 3.39-3.35 (m, 4H), 1.51-1.37 (m, 4H), 1.31-1.26 (m, 4H), 1.20 (t, J=7.1 Hz, 3H).

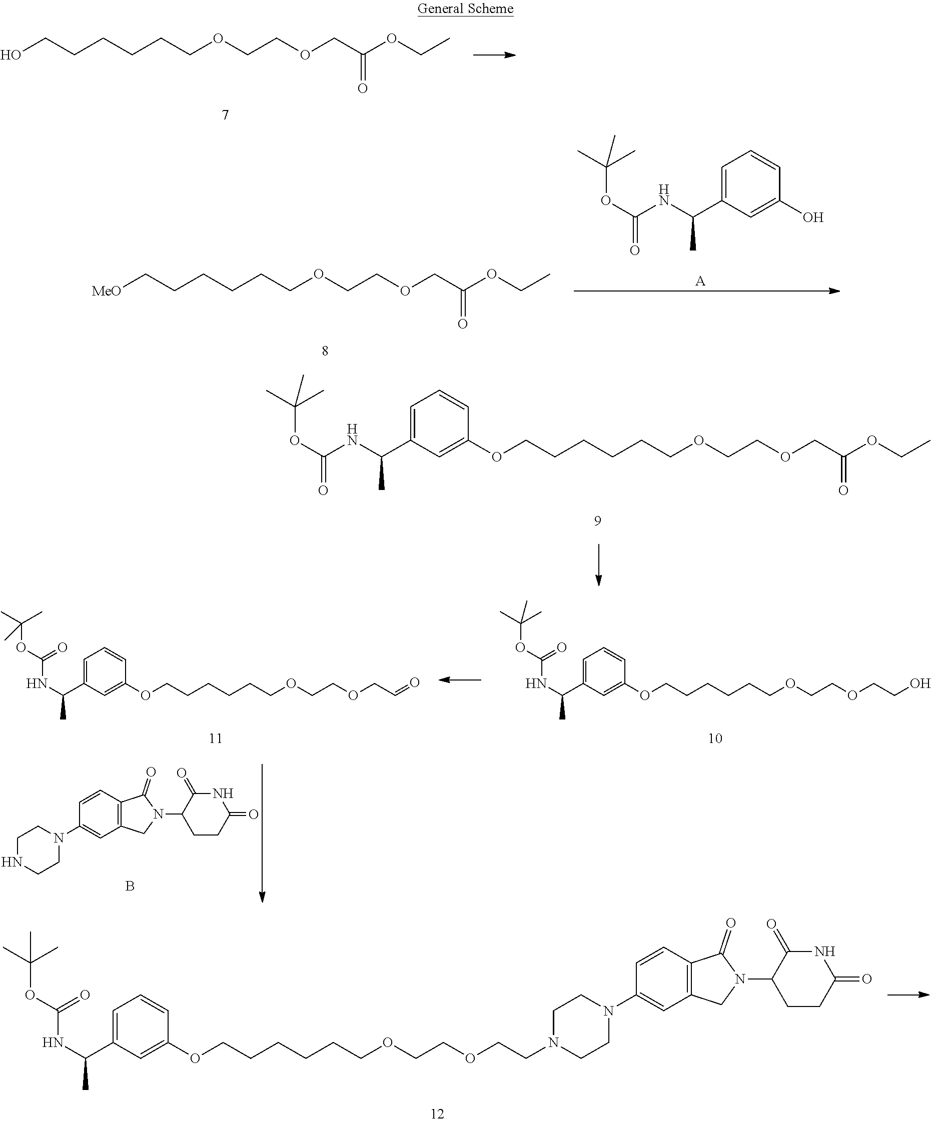

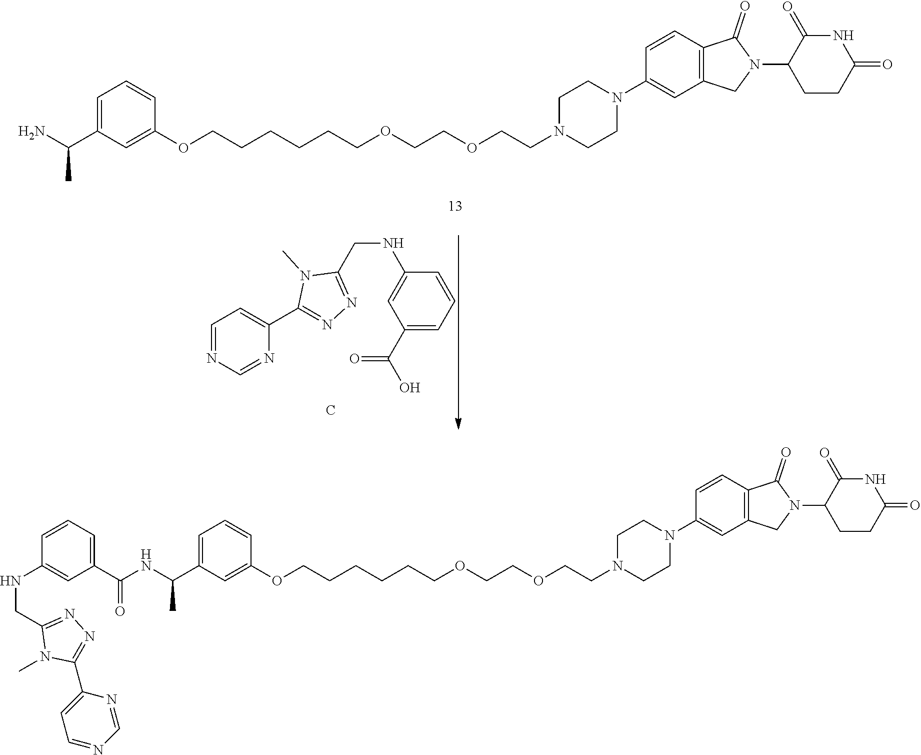

### General Procedures

Compound 8. A solution of compound 7 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 9. A mixture of compound A (2.5 mmol, 1 eq), compound 8 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 9.

Compound 10. To a mixture of compound 9 (0.186 mmol) in Et2O (5 mL), LiBH₄ (0.279 mmol) was added, followed by methanol (0.279 mmol) at 0° C. The reaction mixture was stirred at ambient temperature overnight. Solvents were evaporated and the residue was partitioned between DCM (5 ml) and water, contains with citric acid (0.7 g, 3.64 mmol in water 1 ml). The extract was separated and dried over Na₂SO₄. The crude product 10 was used further without additional purification.

Compound 11. A flask was charged with 10 mL of dichloromethane and oxalyl chloride (1.14 mmol). The solution was stirred and cooled to −60° C. then dimethyl sulfoxide (1.69 mmol) in 5 mL of dichloromethane was added dropwise at a rapid rate. After 5 min compound 10 (0.22 mmol) in 5 ml of dichloromethane was added dropwise maintaining the temperature at −50 to −60° C. After another 15 min, triethylamine (0.5 ml, 3.59 mmol) was added dropwise while keeping the temperature at or below −50° C. Stirring was continued for 5 min, after which time the mixture was allowed to warm to room temperature and 10 mL of water is added. The aqueous layer was separated and extracted with two 10 ml portions of dichloromethane. The organic layers were combined and dried over anhydrous magnesium sulfate. Rotary evaporation of the solvents gave 0.115 g (95%) of crude product 11 that used further without additional purification.

Compound 12. To a suspension of B (or other recruiter) (0.22 mmol) and compound 11 (0.22 mmol) in 10 mL of DCM was added DIPEA (0.1 mL, 0.67 mmol) and the mixture was stirred for an hour. Then sodium triacetoxyborohydride (0.191 g, 0.9 mmol) was added. The suspension was stirred at room temperature for 12 h and quenched with saturated NaHCO₃ aqueous solution. The product was extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 12.

Compound 13. A solution of compound 12 (1.2 mmol, 1 eq) in dry DCM (50 mL) with 1.2 ml 3 M HCl in dioxane (5 eq) were stirred for 24 h at r.t. The mixture was evaporated to dryness to give the crude product 13 which was used in the next step without additional purification

D135. A mixture of compound 13 (0.1 mmol, 1 eq), compound C (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was purified by HPLC in acidic conditions (YMC-Pack ODS-AQ 250×20 mml, S-10 μm, 12 nm, gradient water-acetonitrile).

### Syntheses According to the General Procedures

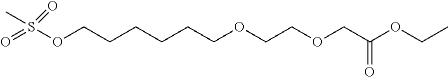 [SMILES: CCOC(=O)COCCOCCCCCCOS(C)(=O)=O]

Compound 8. ethyl 3,10,13-trioxa-2-thiapentadecan-15-oate 2,2-dioxide. Colorless oil 98%.

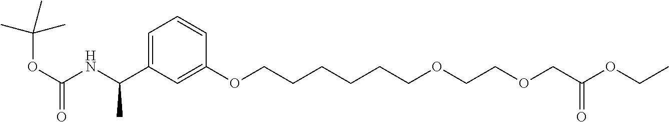 [SMILES: CCOC(=O)COCCOCCCCCCOc1cccc([C@@H](C)NC(=O)OC(C)(C)C)c1]

Compound 9. (R)-ethyl 2-(2-(6-(3-(1-(tert-butoxycarbonylamino)ethyl)phenoxy) hexyloxy)ethoxy)acetate. Colorless oil 69%. LCMS (C18 column 20×2 mm, 2.5 μm, pore size 100 Å, water-acetonitrile+0.1% TFA, gradient 5 to 87% for 3 min, retention time 1.91 min). MS (ESI) m/z 468.9 [MH]+. ¹H NMR (400 MHz, DMSO) δ 7.32 (d, J=8.3 Hz, 1H), 7.18 (t, J=7.8 Hz, 1H), 6.84-6.82 (m, 2H), 6.74 (dd, J=7.8, 1.6 Hz, 1H), 4.56 (m 1H), 4.12-4.08 (m, 4H), 3.92 (t, J=6.4 Hz, 2H), 3.60-3.57 (m, 2H), 3.50-3.47 (m, 2H), 1.77-1.66 (m, 2H), 1.54-1.47 (m, 2H), 1.43-1.21 (m, 20H).

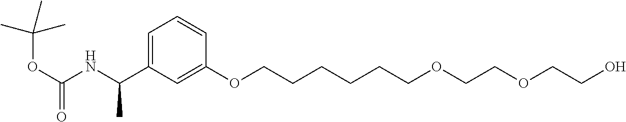 [SMILES: C[C@@H](NC(=O)OC(C)(C)C)c1cccc(OCCCCCCOCCOCCO)c1]

Compound 10. (R)-tert-butyl 1-(3-(6-(2-(2-hydroxyethoxy)ethoxy)hexyloxy)phenyl)ethylcarbamate. Yield 97%. LCMS (C18 column 20×2 mm, 2.5 μm, pore size 100 Å, water-acetonitrile+0.1% TFA, gradient 5 to 87% for 3 min, retention time 1.62 min). MS (ESI) m/z 426.4 [MH]+. ¹H NMR (400 MHz, DMSO) δ 7.30 (t, J=8.1 Hz, 1H), 6.85-6.82 (m, 2H), 6.75 (dd, J=8.2, 2.2 Hz, 1H), 4.59-4.52 (m, 2H), 3.93 (t, J=6.3 Hz, 2H), 3.52-3.46 (m, 6H), 3.43-3.37 (m, 4H), 1.73-1.67 (m, 2H), 1.55-1.48 (m, 2H), 1.44-1.27 (m, 16H

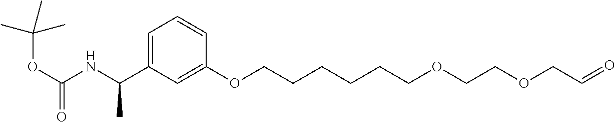 [SMILES: C[C@@H](NC(=O)OC(C)(C)C)c1cccc(OCCCCCCOCCOCC=O)c1]

Compound 11. (R)-tert-butyl 1-(3-(6-(2-(2-oxoethoxy)ethoxy)hexyloxy)phenyl)ethylcarbamate. Yield 73%. LCMS (C18 column 20×2 mm, 2.5 μm, pore size 100 Å, water-acetonitrile+0.1% TFA, gradient 5 to 87% for 3 min, retention time 1.48 min). MS (ESI) m/z 424.5 [MH]+.

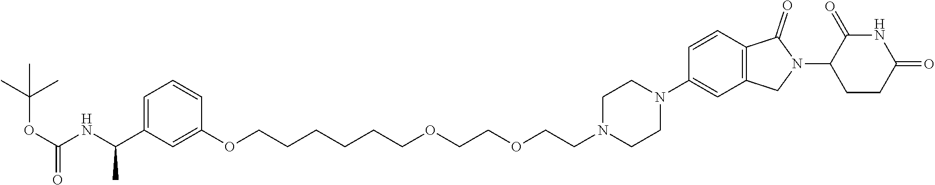 [SMILES: C[C@@H](NC(=O)OC(C)(C)C)c1cccc(OCCCCCCOCCOCCN2CCN(c3ccc4c(c3)CN(C3CCC(=O)NC3=O)C4=O)CC2)c1]

Compound 12. tert-butyl (1R)-1-(3-(6-(2-(2-(4-(2-(2,6-dioxopiperidin-3-yl)-1-oxoisoindolin-5-yl)piperazin-1-yl)ethoxy)ethoxy)hexyloxy)phenyl)ethylcarbamate. Yield 68%. LCMS (C18 column 100×4.6 mm, 5.0 μm, pore size 100 Å, water-acetonitrile+0.1% TFA, gradient 5 to 87% for 10 min, retention time 6.95 min). MS (ESI) m/z 736.8 [MH]+.

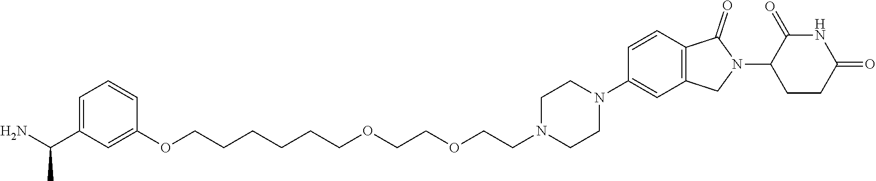 [SMILES: C[C@@H](N)c1cccc(OCCCCCCOCCOCCN2CCN(c3ccc4c(c3)CN(C3CCC(=O)NC3=O)C4=O)CC2)c1]

Compound 13. 3-(5-(4-(2-(2-(6-(3-((R)-1-aminoethyl)phenoxy)hexyloxy)ethoxy)ethyl)piperazin-1-yl)-1-oxoisoindolin-2-yl)piperidine-2,6-dione hydrochloride. Yield 97%. LCMS (C18 column 100×4.6 mm, 5.0 μm, pore size 100 Å, water-acetonitrile+0.1% TFA, gradient 5 to 87% for 10 min, retention time 4.27 min). MS (ESI) m/z 636.5 [MH]+.

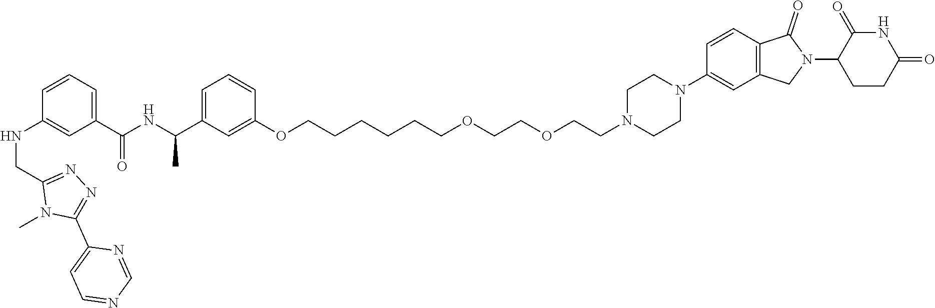 [SMILES: C[C@@H](NC(=O)c1cccc(NCc2nnc(-c3ccncn3)n2C)c1)c1cccc(OCCCCCCOCCOCCN2CCN(c3ccc4c(c3)CN(C3CCC(=O)NC3=O)C4=O)CC2)c1]

D135. N-((1R)-1-(3-(6-(2-(2-(4-(2-(2,6-dioxopiperidin-3-yl)-1-oxoisoindolin-5-yl)piperazin-1-yl)ethoxy)ethoxy)hexyloxy)phenyl)ethyl)-3-((4-methyl-5-(pyrimidin-4-yl)-4H-1,2,4-triazol-3-yl)methylamino)benzamide. Yield 53%. LCMS (C18 column 100×4.6 mm, 5.0 μm, pore size 100 Å, water-acetonitrile+0.1% TFA, gradient 5 to 87% for 10 min, retention time 5.03 min). MS (ESI) m/z 928.8 [MH]+. ¹H NMR (400 MHz, DMSO-d₆) δ 10.93 (s, 1H), 9.75 (br, 1H), 9.33 (s, 1H), 8.97 (d, J=5.3 Hz, 1H), 8.56 (d, J=8.2 Hz, 1H), 8.18 (dd, J=5.3, 1.4 Hz, 1H), 7.57 (d, J=11.3 Hz, 1H), 7.22-7.09 (m, 6H), 6.94-6.89 (m, 3H), 6.75 (dd, J=7.8, 2.2 Hz, 1H), 5.12-5.03 (m, 2H), 4.57 (s, 2H), 4.36-4.20 (m, 2H), 4.07 (s, 3H), 4.00 (d, 2H), 3.92 (t, J=6.5 Hz, 2H), 3.78 (t, J=4.3 Hz, 2H), 3.61-3.51 (m, 11H), 3.23 (br, 4H), 2.94-2.85 (m, 1H), 2.67-2.55 (m, 1H), 2.42-2.31 (m, 1H), 1.99-1.91 (m, 1H), 1.70-1.66 (m, 2H), 1.54-1.31 (m, 9H).

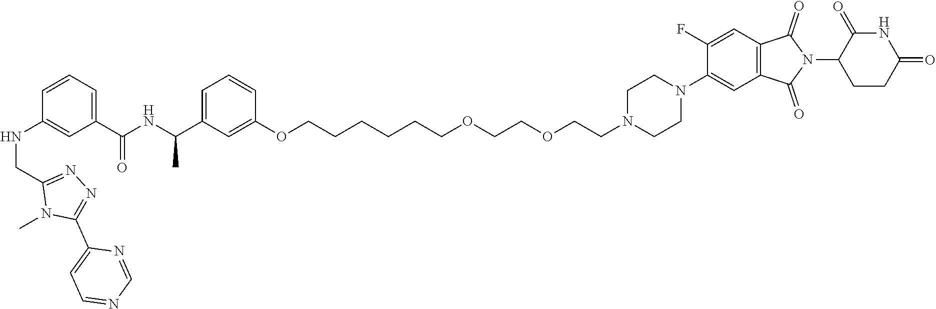 [SMILES: C[C@@H](NC(=O)c1cccc(NCc2nnc(-c3ccncn3)n2C)c1)c1cccc(OCCCCCCOCCOCCN2CCN(c3cc4c(cc3F)C(=O)N(C3CCC(=O)NC3=O)C4=O)CC2)c1]

D140. N-((1R)-1-(3-(6-(2-(2-(4-(2-(2,6-dioxopiperidin-3-yl)-6-fluoro-1,3-dioxoisoindolin-5-yl)piperazin-1-yl)ethoxy)ethoxy)hexyloxy)phenyl)ethyl)-3-((4-methyl-5-(pyrimidin-4-yl)-4H-1,2,4-triazol-3-yl)methylamino)benzamide. Yield 49%. LCMS (C18 column 100×4.6 mm, 5.0 μm, pore size 100 Å, water-acetonitrile+0.1% TFA, gradient 5 to 87% for 10 min, retention time 5.13 min). MS (ESI) m/z 961.1 [MH]+. ¹H NMR (400 MHz, DMSO-d₆) δ 11.08 (s, 1H), 9.33 (s, 1H), 8.96 (d, J=5.3 Hz, 1H), 8.54 (d, J=8.2 Hz, 1H), 8.18 (dd, J=5.3, 1.4 Hz, 1H), 7.71 (d, J=11.3 Hz, 1H), 7.44 (d, J=7.4 Hz, 1H), 7.21-7.09 (m, 4H), 6.93-6.88 (m, 3H), 6.75 (dd, J=7.8, 2.2 Hz, 1H), 6.44 (t, J=5.5 Hz, 1H), 5.12-5.05 (m, 2H), 4.56 (d, J=5.6 Hz, 2H), 4.07 (s, 3H), 3.92 (t, J=6.5 Hz, 2H), 3.55-3.46 (m, 6H), 3.38 (t, J=6.5 Hz, 2H), 3.22 (br, 4H), 2.93-2.82 (m, 1H), 2.67-2.54 (m, 5H), 2.06-1.99 (m, 1H), 1.70-1.64 (m, 2H), 1.53-1.31 (m, 12H).

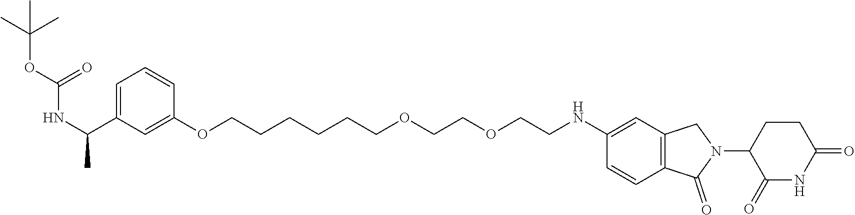 [SMILES: C[C@@H](NC(=O)OC(C)(C)C)c1cccc(OCCCCCCOCCOCCNc2ccc3c(c2)CN(C2CCC(=O)NC2=O)C3=O)c1]

Compound 12. tert-butyl (1R)-1-(3-(6-(2-(2-(2-(2,6-dioxopiperidin-3-yl)-1-oxoisoindolin-5-ylamino)ethoxy)ethoxy)hexyloxy)phenyl)ethylcarbamate. Yield 40%. LCMS (C18 column 100×4.6 mm, 5.0 μm, pore size 100 Å, water-acetonitrile+0.1% TFA, gradient 5 to 87% for 10 min, retention time 6.99 min). MS (ESI) m/z 668.0[MH]+.

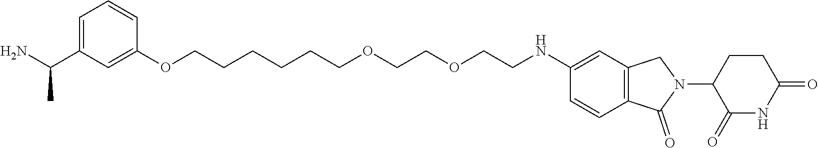 [SMILES: C[C@@H](N)c1cccc(OCCCCCCOCCOCCNc2ccc3c(c2)CN(C2CCC(=O)NC2=O)C3=O)c1]

Compound 13. 3-(5-(2-(2-(6-(3-((R)-1-aminoethyl)phenoxy)hexyloxy)ethoxy)ethylamino)-1-oxoisoindolin-2-yl)piperidine-2,6-dione hydrochloride. Yield 96%. LCMS (C18 column 100×4.6 mm, 5.0 μm, pore size 100 Å, water-acetonitrile+0.1% TFA, gradient 5 to 87% for 10 min, retention time 4.59 min). MS (ESI) m/z 567.3 [MH]+.

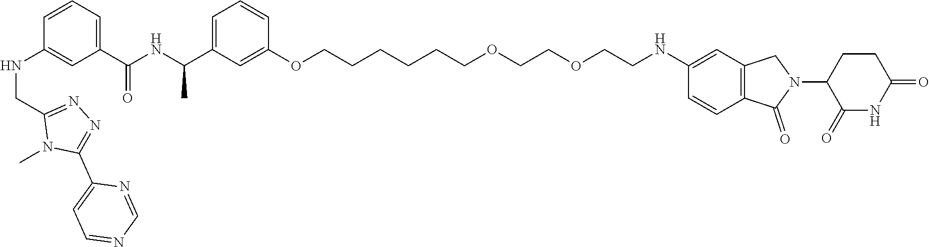 [SMILES: C[C@@H](NC(=O)c1cccc(NCc2nnc(-c3ccncn3)n2C)c1)c1cccc(OCCCCCCOCCOCCNc2ccc3c(c2)CN(C2CCC(=O)NC2=O)C3=O)c1]

D141. N-((1R)-1-(3-(6-(2-(2-(2-(2,6-dioxopiperidin-3-yl)-1-oxoisoindolin-5-ylamino)ethoxy)ethoxy)hexyloxy)phenyl)ethyl)-3-((4-methyl-5-(pyrimidin-4-yl)-4H-1,2,4-triazol-3-yl)methylamino)benzamide. Yield 47%. LCMS (C18 column 100×4.6 mm, 5.0 μm, pore size 100 Å, water-acetonitrile+0.1% TFA, gradient 5 to 87% for 10 min, retention time 6.52 min). MS (ESI) m/z 860.0 [MH]+. ¹H NMR (400 MHz, DMSO-d₆) δ 10.90 (s, 1H), 9.33 (s, 1H), 8.96 (d, J=5.3 Hz, 1H), 8.54 (d, J=8.2 Hz, 1H), 8.18 (dd, J=5.3, 1.4 Hz, 1H), 7.37 (d, J=8.3 Hz, 1H), 7.22-7.09 (m, 4H), 6.93-6.89 (m, 3H), 6.75 (dd, J=7.8, 2.2 Hz, 1H), 6.69-6.66 (m, 2H), 6.45 (t, J=5.5 Hz, 1H), 6.34 (t, J=5.8 Hz, 1H), 5.13-5.03 (m, 2H), 4.56 (d, J=5.6 Hz, 2H), 4.28-4.11 (m, 2H), 4.06 (s, 3H), 3.91 (t, J=6.5 Hz, 2H), 3.57 (t, J=6.5 Hz, 2H), 3.55-3.47 (m, 4H), 3.36 (t, J=6.5 Hz, 2H), 3.27-3.23 (m, 2H), 2.92-2.83 (m, 1H), 2.67-2.57 (m, 1H), 2.38-2.28 (m, 1H), 1.96-1.90 (m, 1H), 1.71-1.64 (m, 2H), 1.52-1.28 (m, 9H).

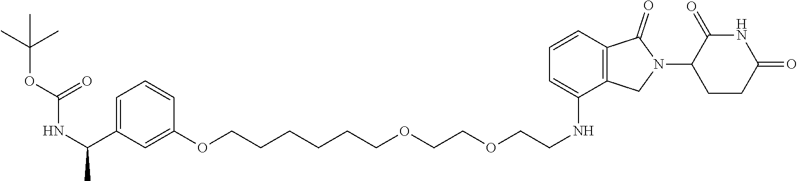 [SMILES: C[C@@H](NC(=O)OC(C)(C)C)c1cccc(OCCCCCCOCCOCCNc2cccc3c2CN(C2CCC(=O)NC2=O)C3=O)c1]

Compound 12. tert-butyl (1R)-1-(3-(6-(2-(2-(2-(2,6-dioxopiperidin-3-yl)-1-oxoisoindolin-4-ylamino)ethoxy)ethoxy)hexyloxy)phenyl)ethylcarbamate. Yield 40%. LCMS (C18 column 100×4.6 mm, 5.0 μm, pore size 100 Å, water-acetonitrile+0.1% TFA, gradient 5 to 87% for 10 min, retention time 7.25 min). MS (ESI) m/z 667.9 [MH]+.

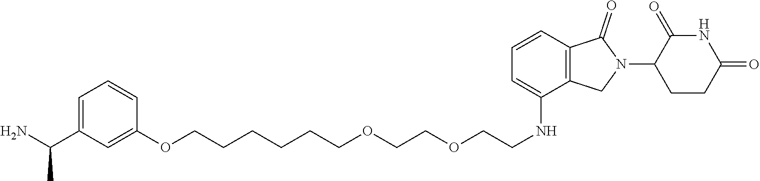 [SMILES: C[C@@H](N)c1cccc(OCCCCCCOCCOCCNc2cccc3c2CN(C2CCC(=O)NC2=O)C3=O)c1]

Compound 13. 3-(4-(2-(2-(6-(3-((R)-1-aminoethyl)phenoxy)hexyloxy)ethoxy)ethylamino)-1-oxoisoindolin-2-yl)piperidine-2,6-dione hydrochloride. Yield 96%. LCMS (C18 column 100×4.6 mm, 5.0 μm, pore size 100 Å, water-acetonitrile+0.1% TFA, gradient 5 to 87% for 10 min, retention time 4.64 min). MS (ESI) m/z 567.7 [MH]+.

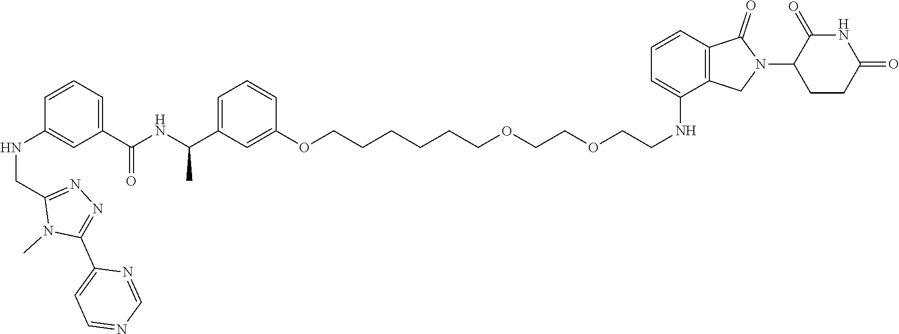 [SMILES: C[C@@H](NC(=O)c1cccc(NCc2nnc(-c3ccncn3)n2C)c1)c1cccc(OCCCCCCOCCOCCNc2cccc3c2CN(C2CCC(=O)NC2=O)C3=O)c1]

D142. N-((1R)-1-(3-(6-(2-(2-(2-(2,6-dioxopiperidin-3-yl)-1-oxoisoindolin-4-ylamino)ethoxy)ethoxy)hexyloxy)phenyl)ethyl)-3-((4-methyl-5-(pyrimidin-4-yl)-4H-1,2,4-triazol-3-yl)methylamino)benzamide. Yield 64% LCMS (C18 column 100×4.6 mm, 5.0 μm, pore size 100 Å, water-acetonitrile+0.1% TFA, gradient 5 to 87% for 10 min, retention time 6.06 min). MS (ESI) m/z 860.0 [MH]+. ¹H NMR (400 MHz, DMSO-d₆) δ 10.99 (s, 1H), 9.33 (s, 1H), 8.97 (d, J=5.3 Hz, 1H), 8.55 (d, J=8.2 Hz, 1H), 8.17 (dd, J=5.3, 1.4 Hz, 1H), 7.27 (t, J=7.8 Hz, 1H), 7.22-7.09 (m, 4H), 6.95-6.89 (m, 4H), 6.80 (d, J=5.3 Hz, 1H), 6.75 (dd, J=7.8, 2.2 Hz, 1H), 6.45 (t, J=5.5 Hz, 1H), 5.56 (t, J=5.8 Hz, 1H), 5.13-5.07 (m, 2H), 4.58 (d, J=5.6 Hz, 2H), 4.24-4.09 (m, 2H), 4.06 (s, 3H), 3.91 (t, J=6.5 Hz, 2H), 3.58 (t, J=6.5 Hz, 2H), 3.55-3.46 (m, 4H), 3.36 (t, J=6.5 Hz, 2H), 3.29 (br, 2H), 2.96-2.87 (m, 1H), 2.67-2.57 (m, 1H), 2.34-2.24 (m, 1H), 2.05-1.99 (m, 1H), 1.69-1.63 (m, 2H), 1.51-1.28 (m, 9H).

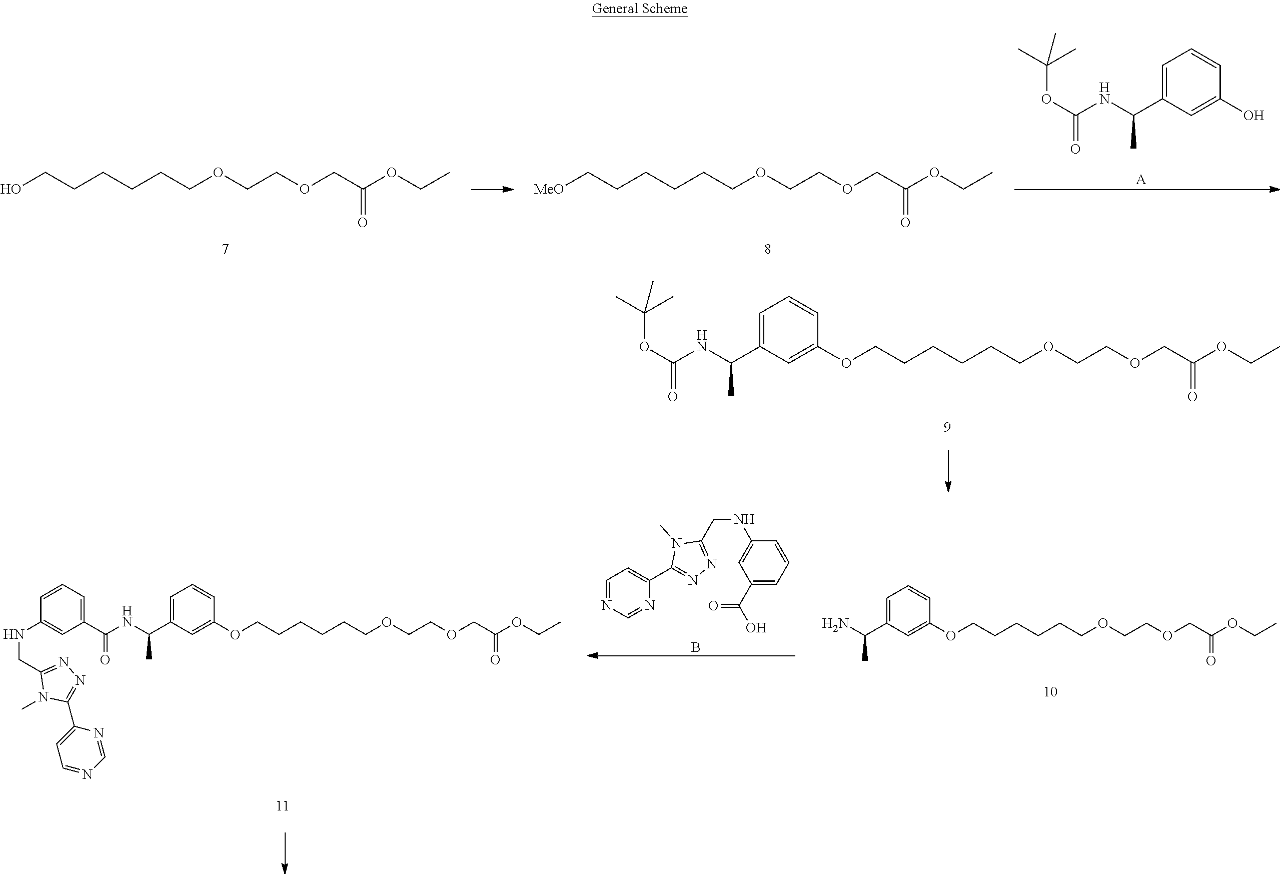

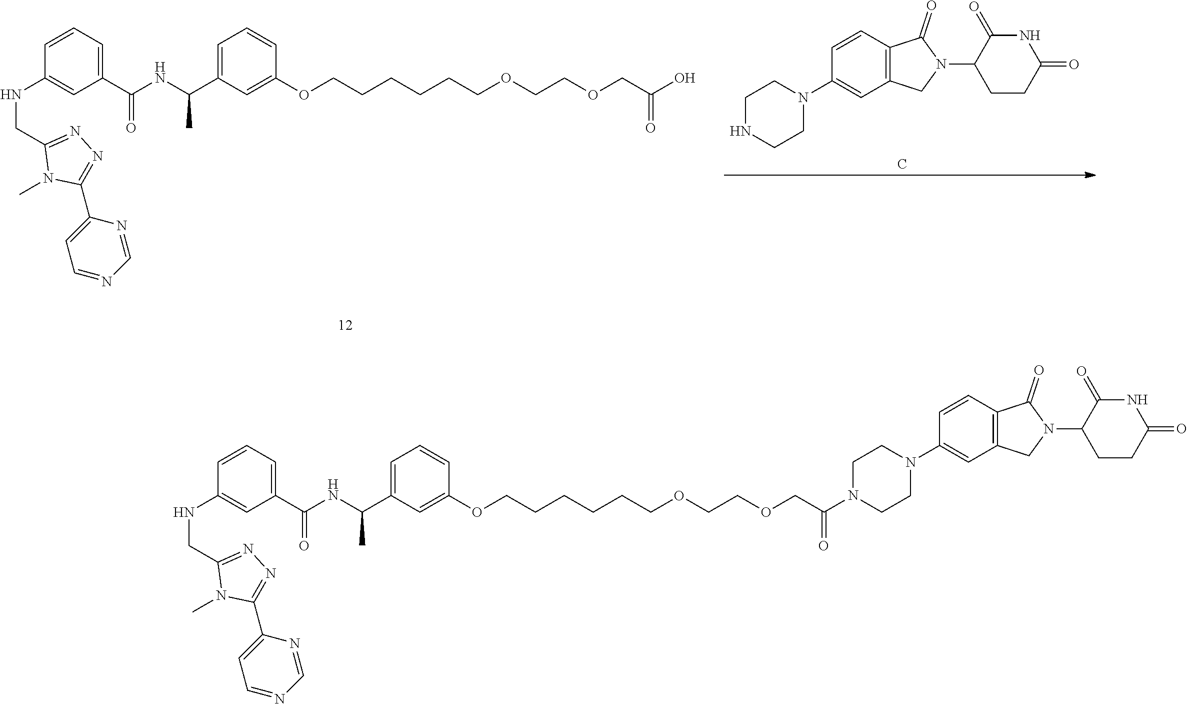

### General Procedures

Compound 8. Solution of compound 7 (1.29 mmol, 1 eq) in CH₂Cl₂ was mixed with DIPEA (1.94 mmol, 1.5 eq) and cooled to 0° C. 1.55 mmol (1.2 eq) of MsCl was added dropwise. The mixture was stirred at room temperature for 2 hours, washed with water, dried over Na₂SO₄ and evaporated. The product was used without purification.

Compound 9. A mixture of compound A (2.5 mmol, 1 eq), compound 8 (2.75 mmol, 1.1 eq), K₂CO₃ (5.0 mmol, 2 eq) and anh. acetonitrile (50 mL) was stirred and heated under reflux for 12 h, filtered, and concentrated under reduced pressure. The residue was purified by silica gel column chromatography using ethyl acetate and hexane (2:1) as an eluent to afford the desired product 11.

Compound 10. A solution of compound 9 (1.2 mmol, 1 eq) in dry DCM (50 mL) with 1.2 ml 3 M HCl in dioxane (5 eq) were stirred for 24 h at r.t. The mixture was evaporated to dryness to give the crude product 12 which was used in the next step without additional purification.

Compound 11. A mixture of compound 10 (0.75 mmol, 1 eq), compound B (0.75 mmol, 1 eq), HOBt (0.9 mmol, 1.2 eq), EDCI (0.9 mmol, 1.2 eq), DIPEA (3.0 mmol, 4 eq) and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 11.

Compound 12. To a solution of compound 11 (0.73 mmol, 1 eq) in MeOH (20 mL) was added a solution KOH (2.2 mmol, 3 eq) in 3 mL H₂O. The mixture was stirred for 12-36 h at ambient temperature and concentrated under reduced pressure. The residue was treated with H₂O (15 mL), and the mixture was acidified to pH 5 with 1N aq. HCl and extracted with of DCM (4*15 mL). Combined organic layers were washed with brine, dried over Na₂SO₄ and evaporated. The product was used for the next step without additional purification.

D138. A mixture of compound 12 (0.1 mmol, 1 eq), compound C (or other recruiter) (0.1 mmol, 1 eq), TBTU (0.2 mmol, 2 eq), DIPEA (0.4 mmol, 4 eq) and anh. pyridine (3 mL) was stirred at ambient temperature for 12 h and concentrated under reduced pressure. The residue was purified by HPLC in neutral conditions (YMC-Pack ODS-AQ 250×20 mml, S-10 μm, 12 nm, gradient water-acetonitrile).

### Synthesis According to General Procedures

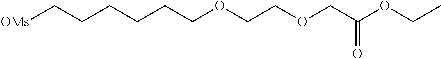 [SMILES: CCOC(=O)COCCOCCCCCCOS(C)(=O)=O]

Compound 8. ethyl 3,10,13-trioxa-2-thiapentadecan-15-oate 2,2-dioxide. Colorless oil 98%.

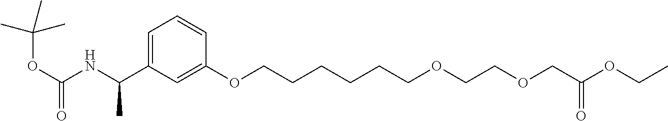 [SMILES: CCOC(=O)COCCOCCCCCCOc1cccc([C@@H](C)NC(=O)OC(C)(C)C)c1]

Compound 9. (R)-ethyl 2-(2-(6-(3-(1-(tert-butoxycarbonylamino)ethyl)phenoxy)hexyloxy) ethoxy)acetate. Colorless oil 69%. LCMS (C18 column 20×2 mm, 2.5 μm, pore size 100 Å, water-acetonitrile+0.1% TFA, gradient 5 to 87% for 3 min, retention time 1.91 min). MS (ESI) m/z 468.9 [MH]+. ¹H NMR (400 MHz, DMSO) δ 7.32 (d, J=8.3 Hz, 1H), 7.18 (t, J=7.8 Hz, 1H), 6.84-6.82 (m, 2H), 6.74 (dd, J=7.8, 1.6 Hz, 1H), 4.56 (m 1H), 4.12-4.08 (m, 4H), 3.92 (t, J=6.4 Hz, 2H), 3.60-3.57 (m, 2H), 3.50-3.47 (m, 2H), 1.77-1.66 (m, 2H), 1.54-1.47 (m, 2H), 1.43-1.21 (m, 20H).

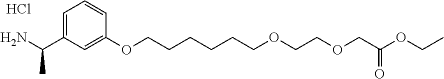 [SMILES: Cl ; CCOC(=O)COCCOCCCCCCOc1cccc([C@@H](C)N)c1]

Compound 10. (R)-ethyl 2-(2-(6-(3-(1-aminoethyl)phenoxy)hexyloxy)ethoxy)acetate hydrochloride Yield 97%. LCMS (C18 column 20×2 mm, 2.5 μm, pore size 100 Å, water-acetonitrile+0.1% TFA, gradient 5 to 87% for 3 min, retention time 1.17 min). MS (ESI) m/z 368.4 [MH]+.

¹H NMR (400 MHz, DMSO) δ 8.57 (br, 2H), 7.30 (t, J=8.1 Hz, 1H), 7.14 (s, 1H), 7.04 (d, J=7.4 Hz, 1H), 6.90 (d, J=8.3 Hz, 1H), 4.32 (m 1H), 4.11-4.08 (m, 4H), 3.97 (t, J=6.3 Hz, 2H), 3.60-3.57 (m, 2H), 3.50-3.47 (m, 2H), 1.75-1.68 (m, 2H), 1.54-1.46 (m, 5H), 1.44-1.31 (m, 4H), 1.19 (t, J=7.1 Hz, 3H)

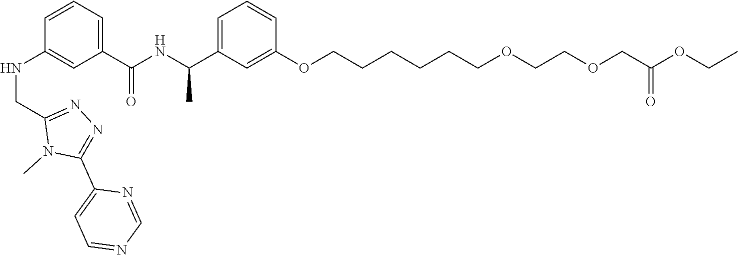 [SMILES: CCOC(=O)COCCOCCCCCCOc1cccc([C@@H](C)NC(=O)c2cccc(NCc3nnc(-c4ccncn4)n3C)c2)c1]

Compound 11. (R)-ethyl 2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)acetate. Yield 52%. LCMS (C18 column 20×2 mm, 2.5 μm, pore size 100 Å, water-acetonitrile+0.1% TFA, gradient 5 to 87% for 3 min, retention time 1.54 min). MS (ESI) m/z 660.5 [MH]+.

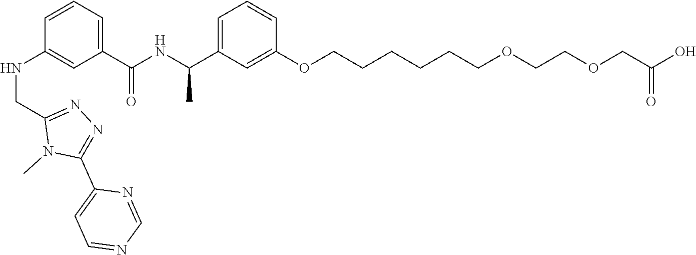 [SMILES: C[C@@H](NC(=O)c1cccc(NCc2nnc(-c3ccncn3)n2C)c1)c1cccc(OCCCCCCOCCOCC(=O)O)c1]

Compound 12. (R)-2-(2-(6-(3-(1-(3-((4-methyl-5-(pyrimidin-4-yl)-4H-1,2,4-triazol-3-yl)methylamino)benzamido)ethyl)phenoxy)hexyloxy)ethoxy)acetic acid. Yield 96%. LCMS (C18 column 20×2 mm, 2.5 μm, pore size 100 Å, water-acetonitrile+0.1% TFA, gradient 5 to 87% for 3 min, retention time 1.31 min). MS (ESI) m/z 632.8 [MH]+.

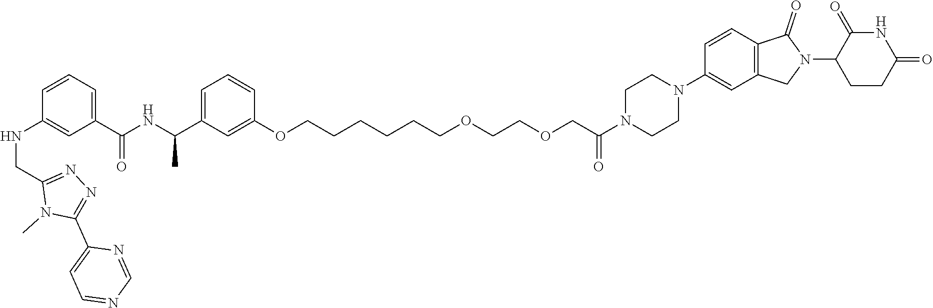 [SMILES: C[C@@H](NC(=O)c1cccc(NCc2nnc(-c3ccncn3)n2C)c1)c1cccc(OCCCCCCOCCOCC(=O)N2CCN(c3ccc4c(c3)CN(C3CCC(=O)NC3=O)C4=O)CC2)c1]

D138. N-((1R)-1-(3-(6-(2-(2-(4-(2-(2,6-dioxopiperidin-3-yl)-1-oxoisoindolin-5-yl)piperazin-1-yl)-2-oxoethoxy)ethoxy)hexyloxy)phenyl)ethyl)-3-((4-methyl-5-(pyrimidin-4-yl)-4H-1,2,4-triazol-3-yl)methylamino)benzamide hydrochloride. Yield 31%. LCMS (C18 column 100×4.6 mm, 5.0 μm, pore size 100 Å, water-acetonitrile+0.1% TFA, gradient 5 to 87% for 10 min, retention time 5.68 min). MS (ESI) m/z 942.9 [MH]+. ¹H NMR (400 MHz, DMSO-d₆) δ 10.93 (s, 1H), 9.32 (s, 1H), 8.96 (d, J=5.3 Hz, 1H), 8.54 (d, J=8.3 Hz, 1H), 8.17 (d, J=5.2 Hz, 1H), 7.52 (d, J=8.7 Hz, 1H), 7.21-7.15 (m, 3H), 7.11-7.05 (m, 3H), 6.93-6.73 (m, 3H), 6.75 (dd, J=7.2, 2.0 Hz, 1H), 6.45 (t, J=5.5 Hz, 1H), 5.12-5.01 (m, 2H), 4.57 (d, J=5.5 Hz, 2H), 4.33-4.19 (m, 4H), 4.06 (s, 3H), 3.90 (t, J=6.5 Hz, 2H), 3.58-3.54 (m, 6H), 3.50-3.48 (m, 2H), 3.37 (m, 3H), 2.93-2.84 (m, 1H), 2.59-2.54 (m, 4H), 2.36-2.30 (m, 1H), 1.95-1.91 (m, 1H), 1.69-1.61 (m, 2H), 1.52-1.30 (m, 9H).

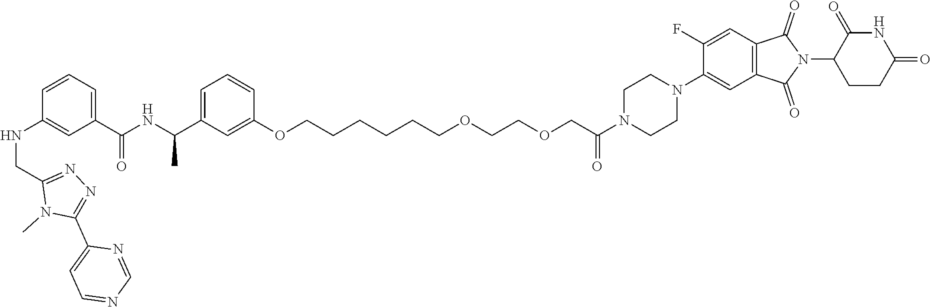 [SMILES: C[C@@H](NC(=O)c1cccc(NCc2nnc(-c3ccncn3)n2C)c1)c1cccc(OCCCCCCOCCOCC(=O)N2CCN(c3cc4c(cc3F)C(=O)N(C3CCC(=O)NC3=O)C4=O)CC2)c1]

D139. N-((1R)-1-(3-(6-(2-(2-(4-(2-(2,6-dioxopiperidin-3-yl)-6-fluoro-1,3-dioxoisoindolin-5-yl)piperazin-1-yl)-2-oxoethoxy)ethoxy)hexyloxy)phenyl)ethyl)-3-((4-methyl-5-(pyrimidin-4-yl)-4H-1,2,4-triazol-3-yl)methylamino)benzamide. Yield 39%. LCMS (C18 column 100×4.6 mm, 5.0 μm, pore size 100 Å, water-acetonitrile+0.1% TFA, gradient 5 to 87% for 10 min, retention time 6.22 min). MS (ESI) m/z 975.2 [MH]+. ¹H NMR (400 MHz, DMSO-d₆) δ 11.09 (s, 1H), 9.32 (s, 1H), 8.97 (d, J=5.3 Hz, 1H), 8.55 (d, J=8.2 Hz, 1H), 8.17 (dd, J=5.3, 1.4 Hz, 1H), 7.75 (d, J=11.3 Hz, 1H), 7.47 (d, J=7.4 Hz, 1H), 7.20-7.09 (m, 4H), 6.92-6.88 (m, 3H), 6.74 (dd, J=7.8, 2.2 Hz, 1H), 6.45 (t, J=5.5 Hz, 1H), 5.13-5.05 (m, 2H), 4.56 (d, J=5.6 Hz, 2H), 4.19 (s, 2H), 4.07 (s, 3H), 3.91 (t, J=6.5 Hz, 2H), 3.60-3.55 (m, 6H), 3.51-3.48 (m, 2H), 3.38 (t, J=6.5 Hz, 2H), 3.26-3.21 (m, 3H), 2.92-2.83 (m, 1H), 2.67-2.54 (m, 3H), 2.06-1.99 (m, 1H), 1.70-1.64 (m, 2H), 1.53-1.31 (m, 9H).

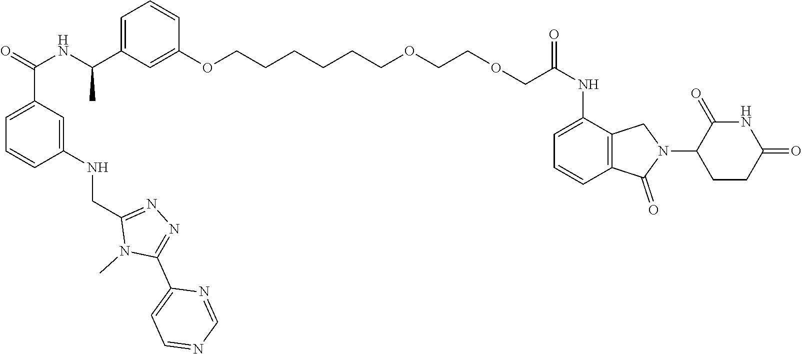 [SMILES: C[C@@H](NC(=O)c1cccc(NCc2nnc(-c3ccncn3)n2C)c1)c1cccc(OCCCCCCOCCOCC(=O)Nc2cccc3c2CN(C2CCC(=O)NC2=O)C3=O)c1]

D170. N-((1R)-1-(3-((6-(2-(2-((2-(2,6-dioxopiperidin-3-yl)-1-oxoisoindolin-4-yl)amino)-2-oxoethoxy)ethoxy)hexyl)oxy)phenyl)ethyl)-3-(((4-methyl-5-(pyrimidin-4-yl)-4H-1,2,4-triazol-3-yl)methyl)amino)benzamide. Yield 35%. LCMS (C18 column 100×4.6 mm, 5.0 μm, pore size 100 Å, water-acetonitrile+0.1% TFA, gradient 5 to 87% for 10 min, retention time 5.69 min). MS (ESI) m/z 874.5 [MH]+. ¹H NMR (400 MHz, DMSO-d₆) δ 10.99 (s, 1H), 9.66 (s, 1H), 9.33 (d, J=1.4 Hz, 1H), 8.96 (d, J=5.2 Hz, 1H), 8.55 (d, J=8.2 Hz, 1H), 8.17 (dd, J=5.3, 1.5 Hz, 1H), 7.73 (dd, J=7.8, 1.2 Hz, 1H), 7.56 (d, J=7.6 Hz, 1H), 7.50 (t, J=7.6 Hz, 1H), 7.19 (m, 3H), 7.10 (d, J=7.4 Hz, 1H), 6.91 (m, 3H), 6.75 (dd, J=8.3, 2.2 Hz, 1H), 6.45 (t, J=5.7 Hz, 1H), 5.12 (m, 2H), 4.57 (d, J=5.7 Hz, 2H), 4.35 (m, 2H), 4.13 (s, 2H), 4.07 (s, 3H), 3.90 (t, J=6.5 Hz, 2H), 3.68 (m, 2H), 3.56 (m, 2H), 3.40 (t, J=6.5 Hz, 2H), 2.91 (m, 1H), 2.59 (m, 1H), 2.35 (m, 1H), 2.00 (m, 1H), 1.64 (p, J=6.6 Hz, 2H), 1.48 (m, 2H), 1.43 (d, J=7.1 Hz, 3H), 1.34 (m, 4H).

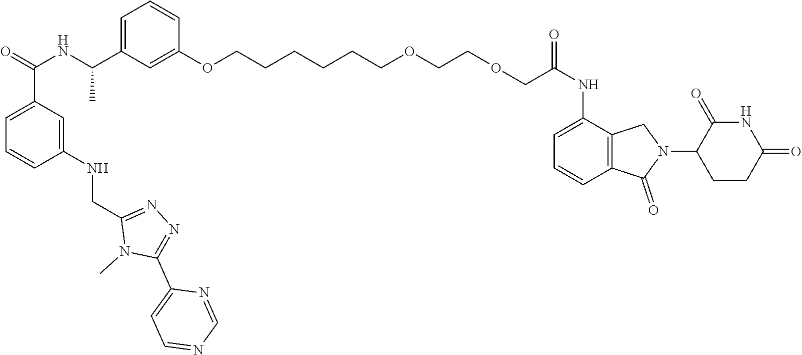 [SMILES: C[C@H](NC(=O)c1cccc(NCc2nnc(-c3ccncn3)n2C)c1)c1cccc(OCCCCCCOCCOCC(=O)Nc2cccc3c2CN(C2CCC(=O)NC2=O)C3=O)c1]

D193. N-((1S)-1-(3-((6-(2-(2-((2-(2,6-dioxopiperidin-3-yl)-1-oxoisoindolin-4-yl)amino)-2-oxoethoxy)ethoxy)hexyl)oxy)phenyl)ethyl)-3-(((4-methyl-5-(pyrimidin-4-yl)-4H-1,2,4-triazol-3-yl)methyl)amino)benzamide. Yield 35%. LCMS (C18 column 100×4.6 mm, 5.0 μm, pore size 100 Å, water-acetonitrile+0.1% TFA, gradient 5 to 87% for 10 min, retention time 5.67 min). MS (ESI) m/z 874.5 [MH]+. ¹H NMR (400 MHz, DMSO-d₆) δ 11.00 (s, 1H), 9.67 (s, 1H), 9.33 (d, J=1.4 Hz, 1H), 8.97 (d, J=5.3 Hz, 1H), 8.56 (d, J=8.2 Hz, 1H), 8.17 (dd, J=5.2, 1.4 Hz, 1H), 7.73 (d, J=8.0 Hz, 1H), 7.56 (d, J=7.3 Hz, 1H), 7.50 (t, J=7.6 Hz, 1H), 7.18 (m, 3H), 7.10 (d, J=7.6 Hz, 1H), 6.91 (m, 3H), 6.75 (d, J=8.9 Hz, 1H), 6.46 (t, J=5.7 Hz, 1H), 5.12 (m, 2H), 4.57 (d, J=5.7 Hz, 2H), 4.35 (m, 2H), 4.13 (s, 2H), 4.07 (s, 3H), 3.89 (t, J=6.5 Hz, 2H), 3.68 (m, 2H), 3.56 (m, 2H), 3.40 (t, J=6.5 Hz, 2H), 2.91 (m, 1H), 2.61 (m, 1H), 2.36 (m, 1H), 2.00 (m, 1H), 1.63 (p, J=6.8 Hz, 2H), 1.49 (m, 2H), 1.43 (d, J=7.1 Hz, 3H), 1.33 (m, 4H).

### Example 26: Preparation of Compound D149, D160, D171, and D198

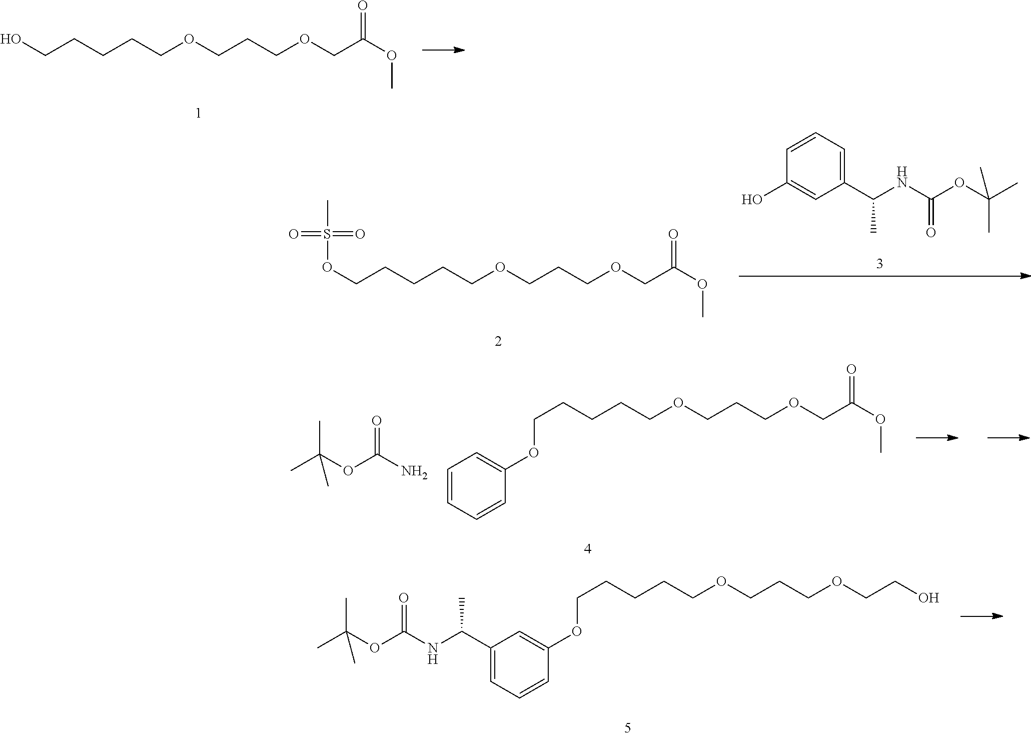

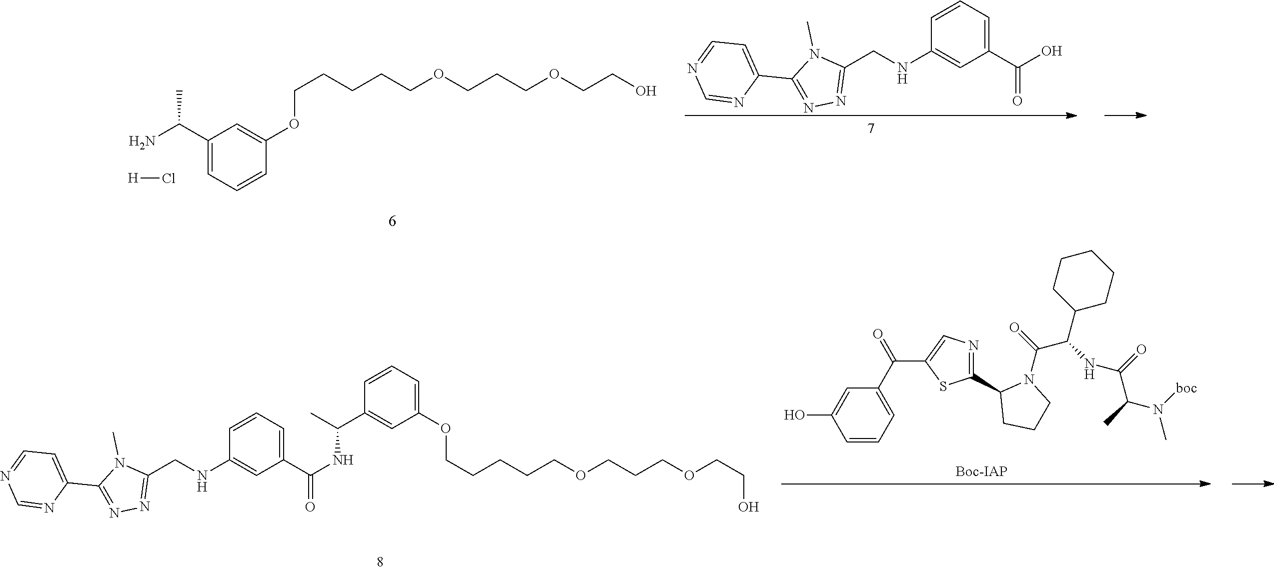

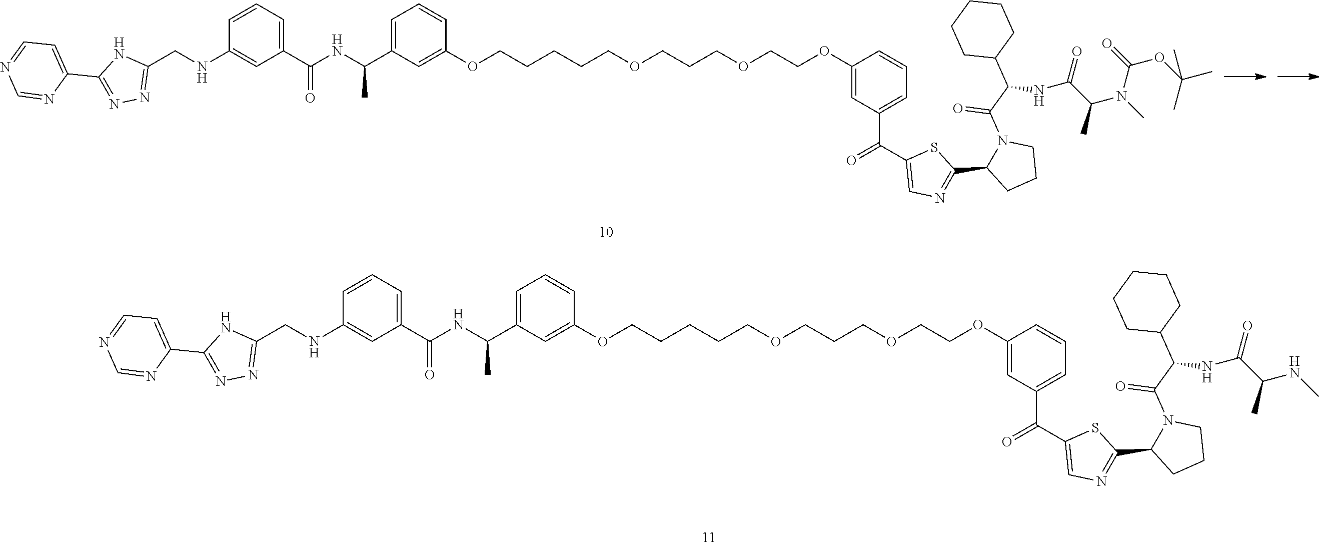 [SMILES: C[C@@H](NC(=O)c1cccc(NCc2nnc(-c3ccncn3)[nH]2)c1)c1cccc(OCCCCCOCCCOCCOc2cccc(C(=O)c3cnc([C@@H]4CCCN4C(=O)[C@@H](NC(=O)[C@H](C)N(C)C(=O)OC(C)(C)C)C4CCCCC4)s3)c2)c1 ; CN[C@@H](C)C(=O)N[C@H](C(=O)N1CCC[C@H]1c1ncc(C(=O)c2cccc(OCCOCCCOCCCCCOc3cccc([C@@H](C)NC(=O)c4cccc(NCc5nnc(-c6ccncn6)[nH]5)c4)c3)c2)s1)C1CCCCC1]

### General Procedures

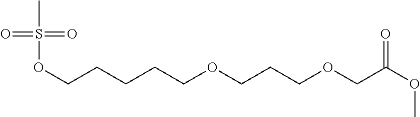 [SMILES: COC(=O)COCCCOCCCCCOS(C)(=O)=O]

Compound 2. 5-(3-(2-methoxy-2-oxoethoxy)propoxy)pentyl methanesulphonate. To an ice cold solution of alcohol 1 (1.3 g, 5.5 mmol) in methylene chloride (10 ml), DIPEA (1.5 ml, 1.12 g, 8.7 mmol) was added, followed by mesyl chloride (0.6 ml, 0.88 g, 7.73 mmol). The reaction mixture was stirred at ambient temperature for 4 hours and quenched with water (10 ml). The organic phase was separated; aqueous was extracted with methylene chloride (10 ml). Combined organic phases were washed with water (10 ml) and dried over MgSO₄. The solvent was stripped off and the product 2 was used further without additional purification. Yield 1.68 g (96%).

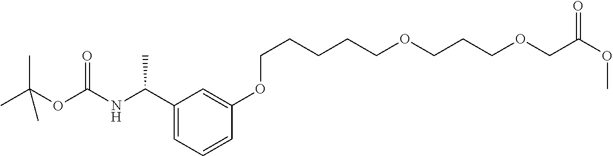 [SMILES: COC(=O)COCCCOCCCCCOc1cccc([C@@H](C)NC(=O)OC(C)(C)C)c1]

Compound 4. (R)-methyl 2-(3-(5-(3-(1-(tert-butoxycarbonylamino)ethyl)phenoxy)pentyloxy) propoxy) acetate. Compound 3 (5.18 mmol, 1 eq), compound 2 (5.37 mmol, 1.03 eq) and K₂CO₃ (1.79 g, 12.97 mmol, 2.5 eq) in dry acetonitrile (50 mL) were stirred for 12 h at reflux. The solvent was evaporated to dryness. The residue was partitioned between methylene chloride (10 ml) and water (10 ml). The organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 2.3 g (97%). LCMS (C18 column 20×2 mm, 2.5 μm, pore size 100 Å, water-acetonitrile+0.1% TFA, gradient 5 to 87% for 3 min, retention time 1.74 min). MS (ESI) m/z 454.3 [MH]+. ¹H NMR (400 MHz, CDCl₃) δ 7.23 (t, J=7.9 Hz, 1H), 6.88 (d, J=7.6 Hz, 1H), 6.84 (s, 1H), 6.78 (dd, J=8.0, 2.0 Hz, 1H), 4.90-4.70 (m, 2H), 4.10 (s, 2H), 3.96 (t, J=6.4 Hz, 2H), 3.76 (s, 3H), 3.63 (t, J=6.4 Hz, 2H), 3.53 (t, J=6.3 Hz, 2H), 3.45 (t, J=6.5 Hz, 2H), 1.90 (p, J=6.3 Hz, 2H), 1.86-1.75 (m, 2H), 1.71-1.59 (m, 2H), 1.59-1.48 (m, 2H), 1.47-1.40 (m, 12H).

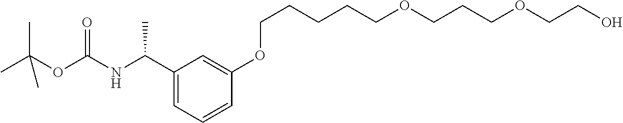 [SMILES: C[C@@H](NC(=O)OC(C)(C)C)c1cccc(OCCCCCOCCCOCCO)c1]

Compound 5. (R)-tert-butyl 1-(3-(5-(3-(2-hydroxyethoxy)propoxy)pentyloxy)phenyl)ethyl carbamate. A mixture of LiBH₄ (0.176 g, 8.0 mmol), compound 4 (2.3 g, 5.0 mmol), methanol (0.33 mL, 8.1 mmol), and ether (10 mL) was refluxed for 15 min. The reaction was quenched with aqueous citric acid (2 g in 10 ml water) with ice-cooling. The mixture was diluted with water and was extracted with dichloromethane. The extract was dried and the solvent was evaporated under reduced pressure. Purification by silica gel (chloroform/methanol=100:1 as developing solvent) afforded compound 5 (2.07 g, 95%).

LCMS (C18 column 20×2 mm, 2.5 μm, pore size 100 Å, water-acetonitrile+0.1% TFA, gradient 5 to 87% for 3 min, retention time 1.64 min). MS (ESI) m/z 426.1 [MH]+. ¹H NMR (400 MHz, CDCl₃) δ 7.24 (t, J=7.9 Hz, 1H), 6.88 (d, J=7.7 Hz, 1H), 6.84 (s, 1H), 6.78 (dd, J=8.1, 1.9 Hz, 1H), 4.90-4.70 (m, 2H), 3.96 (t, J=6.5 Hz, 2H), 3.72 (dd, J=14.5, 9.9 Hz, 2H), 3.60 (t, J=6.3 Hz, 2H), 3.58-3.54 (m, 2H), 3.53 (t, J=6.5 Hz, 2H), 3.45 (t, J=6.4 Hz, 2H), 1.92-1.84 (m, 2H), 1.85-1.77 (m, 2H), 1.72-1.60 (m, 2H), 1.56-1.52 (m, 2H), 1.47-1.40 (m, 13H).

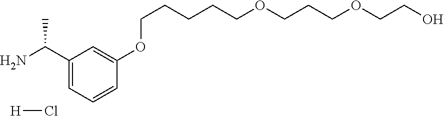

Compound 6. (R)-2-(3-(5-(3-(1-aminoethyl)phenoxy)pentyloxy)propoxy)ethanol hydrochloride To an ice cold solution of compound 5 (0.2 g, 0.47 mmol) in methylene chloride (10 ml) a dioxanic 3M HCl solution (2.7 ml) was added. The mixture was stirred at ambient temperature overnight and then evaporated dryness. The residue was taken up with dry ether (20 ml) and the solid was filtered, washed with ether (10 ml) and dried on air. Yield 0.17 g (100%). LCMS (C18 column 20×2 mm, 2.5 μm, pore size 100 Å, water-acetonitrile+0.1% TFA, gradient 5 to 87% for 3 min, retention time 1.00 min). MS (ESI) m/z 326.5 [MH]+.

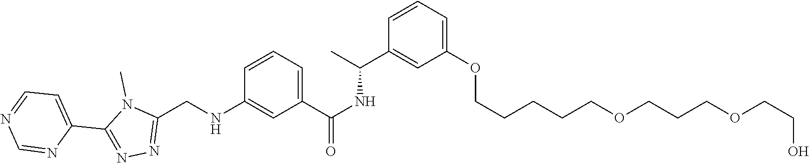 [SMILES: C[C@@H](NC(=O)c1cccc(NCc2nnc(-c3ccncn3)n2C)c1)c1cccc(OCCCCCOCCCOCCO)c1]

Compound 8. (R)—N-(1-(3-(5-(3-(2-hydroxyethoxy)propoxy)pentyloxy)phenyl)ethyl)-3-((4-methyl-5-(pyrimidin-4-yl)-4H-1,2,4-triazol-3-yl)methylamino)benzamide. To an ice cold suspension of amine hydrochloride 6 (0.1 g, 0.27 mmol) in DCM (10 ml), acid 7 (0.09 g, 0.29 mmol) was added, followed by hydroxyl benzotriazole hydrate (0.056 g, 0.36 mmol) and EDCI (0.07 g, 0.36 mmol) and finally DIPEA (0.4 ml, 2.3 mmol). The reaction mixture was stirred at ambient temperature overnight. Then water (5 ml) was added and organic layer was separated. The aqueous layer was extracted with methylene chloride (5 ml). Combined organic layers were dried over MgSO₄ and evaporated. The residue was purified by HPLC in neutral conditions (YMC-Pack ODS-AQ 250×20 mml, S-10 μm, 12 nm, gradient water-acetonitrile). Yield 0.14 g (85%). LCMS (C18 column 20×2 mm, 2.5 μm, pore size 100 Å, water-acetonitrile+0.1% TFA, gradient 5 to 87% for 3 min, retention time 1.32 min). MS (ESI) m/z 618.5 [MH]+.

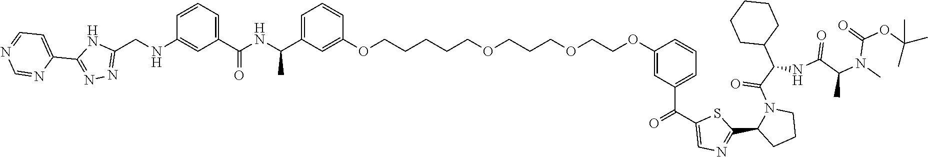 [SMILES: C[C@@H](NC(=O)c1cccc(NCc2nnc(-c3ccncn3)[nH]2)c1)c1cccc(OCCCCCOCCCOCCOc2cccc(C(=O)c3cnc([C@@H]4CCCN4C(=O)[C@@H](NC(=O)[C@H](C)N(C)C(=O)OC(C)(C)C)C4CCCCC4)s3)c2)c1]

Compound 10. tert-butyl (S)-1-((S)-1-cyclohexyl-2-oxo-2-((S)-2-(5-(3-(2-(3-(5-(3-((R)-1-(3-((5-(pyrimidin-4-yl)-4H-1,2,4-triazol-3-yl)methylamino)benzamido)ethyl)phenoxy)pentyloxy) propoxy)ethoxy)benzoyl)thiazol-2-yl)pyrrolidin-1-yl)ethylamino)-1-oxopropan-2-yl(methyl) carbamate. To a solution of compound 8 (0.13 g, 0.2 mmol), Boc-IAP (0.11 g, 0.2 mmol) and triphenylphosphine (0.07 g, 0.26 mmol) in THF, (E)-diisopropyl diazene-1,2-dicarboxylate (0.06 ml, 0.06 g, 0.03 mmol) was added. The mixture was stirred at ambient temperature overnight, concentrated in vacuo. The product 10 was purified by HPLC in neutral conditions (YMC-Pack ODS-AQ 250×20 mml, S-10 μm, 12 nm, gradient water-acetonitrile). Yield 0.15 g (61%).

LCMS (C18 column 20×2 mm, 2.5 μm, pore size 100 Å, water-acetonitrile+0.1% TFA, gradient 5 to 87% for 10 min, retention time 8.55 min). MS (ESI) m/z 1198.8 [MH]+.

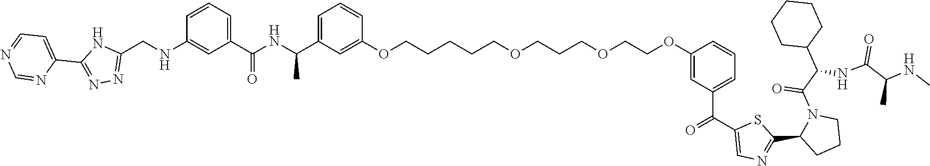 [SMILES: CN[C@@H](C)C(=O)N[C@H](C(=O)N1CCC[C@H]1c1ncc(C(=O)c2cccc(OCCOCCCOCCCCCOc3cccc([C@@H](C)NC(=O)c4cccc(NCc5nnc(-c6ccncn6)[nH]5)c4)c3)c2)s1)C1CCCCC1]

Compound 11. N-((R)-1-(3-(5-(3-(2-(3-(2-((S)-1-((S)-2-cyclohexyl-2-((S)-2-(methylamino) propanamido)acetyl)pyrrolidin-2-yl)thiazole-5-carbonyl)phenoxy)ethoxy)propoxy)pentyloxy) phenyl)ethyl)-3-((5-(pyrimidin-4-yl)-4H-1,2,4-triazol-3-yl)methylamino)benzamide. A solution of compound 10 (0.15 g, 0.13 mmol) in a mixture isopropanol-DCM (10%, 5 ml) was treated with 3M dioxanic HCl (0.2 ml). The reaction was evaporated in vacuo and treated with concentrated aqueous solution of NaHCO₃ (1 ml). The product 11 was extracted with DCM, concentrated and purified by HPLC in neutral conditions (YMC-Pack ODS-AQ 250×20 mml, S-10 μm, 12 nm, gradient water-acetonitrile). Yield 0.021 g (13%). LCMS (C18 column 20×2 mm, 2.5 μm, pore size 100 Å, water-acetonitrile+0.1% TFA, gradient 5 to 87% for 10 min, retention time 5.75 min). MS (ESI) m/z 1099.2 [MH]+. ¹H NMR (400 MHz, DMSO-d₆) δ 9.32 (d, J=1.4 Hz, 1H), 8.96 (d, J=5.3 Hz, 1H), 8.54 (d, J=8.2 Hz, 1H), 8.47 (s, 1H), 8.17 (dd, J=5.2, 1.4 Hz, 1H), 7.89 (d, J=8.8 Hz, 1H), 7.65 (m, 2H), 7.45 (t, J=7.9 Hz, 1H), 7.24 (dd, J=8.1, 2.6 Hz, 1H), 7.18 (m, 3H), 7.10 (d, J=7.6 Hz, 1H), 6.91 (m, 3H), 6.74 (m, 1H), 6.44 (t, J=5.7 Hz, 1H), 5.38 (dd, J=8.0, 3.0 Hz, 1H), 5.09 (p, J=7.1 Hz, 1H), 4.57 (d, J=5.6 Hz, 2H), 4.49 (t, J=7.8 Hz, 1H), 4.15 (t, J=4.6 Hz, 2H), 4.07 (s, 3H), 3.91 (t, J=6.5 Hz, 2H), 3.78 (m, 2H), 3.71 (t, J=4.7 Hz, 2H), 3.50 (t, J=6.4 Hz, 3H), 3.38 (t, J=6.4 Hz, 3H), 3.33 (t, J=6.8 Hz, 2H), 2.97 (q, J=6.8 Hz, 1H), 2.28 (m, 1H), 2.18 (s, 3H), 2.02 (m, 2H), 1.67 (m, 7H), 1.52 (m, 4H), 1.43 (m, 5H), 1.07 (m, 9H).

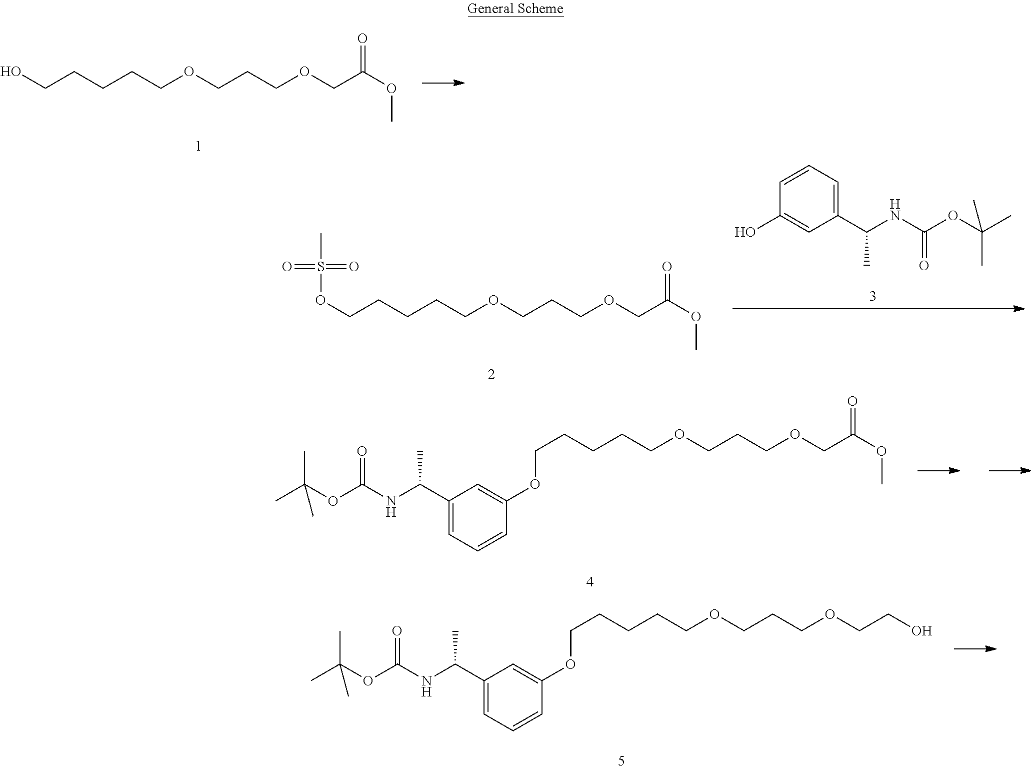

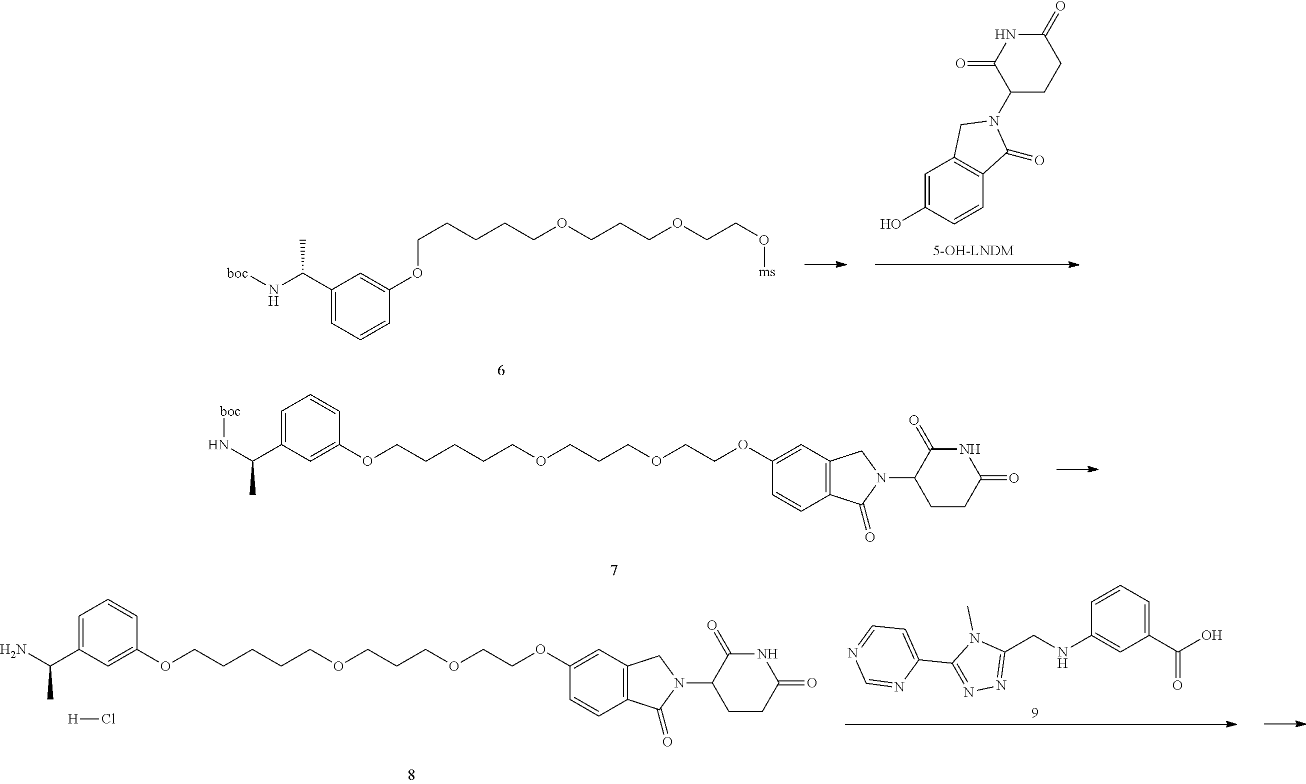

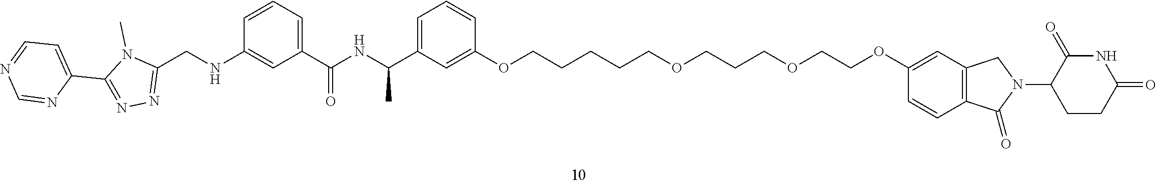 [SMILES: C[C@@H](NC(=O)c1cccc(NCc2nnc(-c3ccncn3)n2C)c1)c1cccc(OCCCCCOCCCOCCOc2ccc3c(c2)CN(C2CCC(=O)NC2=O)C3=O)c1]

### General Procedures

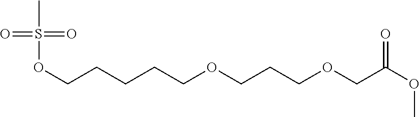 [SMILES: COC(=O)COCCCOCCCCCOS(C)(=O)=O]

Compound 2. 5-(3-(2-methoxy-2-oxoethoxy)propoxy)pentyl methanesulphonate. To an ice cold solution of alcohol 1 (1.3 g, 5.5 mmol) in methylene chloride (10 ml), DIPEA (1.5 ml, 1.12 g, 8.7 mmol) was added, followed by mesyl chloride (0.6 ml, 0.88 g, 7.73 mmol). The reaction mixture was stirred at ambient temperature for 4 hours and quenched with water (10 ml). Organic phase was separated; aqueous was extracted with methylene chloride (10 ml). Combined organic phases were washed with water (10 ml) and dried over MgSO₄. The solvent was stripped off and the product 2 was used further without additional purification. Yield 1.68 g (96%).

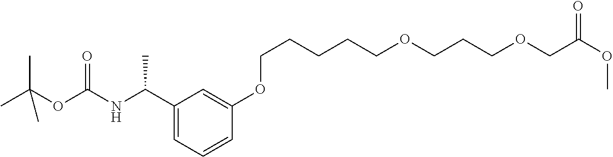 [SMILES: COC(=O)COCCCOCCCCCOc1cccc([C@@H](C)NC(=O)OC(C)(C)C)c1]

Compound 4. (R)-methyl 2-(3-(5-(3-(1-(tert-butoxycarbonylamino)ethyl)phenoxy)pentyloxy) propoxy) acetate. Compound 3 (5.18 mmol, 1 eq), compound 2 (5.37 mmol, 1.03 eq) and K₂CO₃ (1.79 g, 12.97 mmol, 2.5 eq) in dry acetonitrile (50 mL) were stirred for 12 h at reflux. The solvent was evaporated to dryness. The residue was partitioned between methylene chloride (10 ml) and water (10 ml). Organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 2.3 g (97%). LCMS (C18 column 20×2 mm, 2.5 μm, pore size 100 Å, water-acetonitrile+0.1% TFA, gradient 5 to 87% for 3 min, retention time 1.74 min). MS (ESI) m/z 454.3 [MH]+. ¹H NMR (400 MHz, CDCl₃) δ 7.23 (t, J=7.9 Hz, 1H), 6.88 (d, J=7.6 Hz, 1H), 6.84 (s, 1H), 6.78 (dd, J=8.0, 2.0 Hz, 1H), 4.90-4.70 (m, 2H), 4.10 (s, 2H), 3.96 (t, J=6.4 Hz, 2H), 3.76 (s, 3H), 3.63 (t, J=6.4 Hz, 2H), 3.53 (t, J=6.3 Hz, 2H), 3.45 (t, J=6.5 Hz, 2H), 1.90 (p, J=6.3 Hz, 2H), 1.86-1.75 (m, 2H), 1.71-1.59 (m, 2H), 1.59-1.48 (m, 2H), 1.47-1.40 (m, 12H).

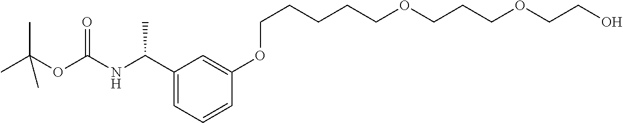 [SMILES: C[C@@H](NC(=O)OC(C)(C)C)c1cccc(OCCCCCOCCCOCCO)c1]

Compound 5. (R)-tert-butyl 1-(3-(5-(3-(2-hydroxyethoxy)propoxy)pentyloxy)phenyl)ethyl carbamate. A mixture of LiBH₄ (0.176 g, 8.0 mmol), compound 4 (2.3 g, 5.0 mmol), methanol (0.33 mL, 8.1 mmol), and ether (10 mL) was refluxed for 15 min. The reaction was quenched with aqueous citric acid (2 g in 10 ml water) with ice-cooling. The mixture was diluted with water and was extracted with dichloromethane. The extract was dried over Na₂SO₄ and the solvent was evaporated under reduced pressure. Purification by silica gel (chloroform/methanol=100:1 as developing solvent) afforded compound 5 (2.07 g, 95%).

LCMS (C18 column 20×2 mm, 2.5 μm, pore size 100 Å, water-acetonitrile+0.1% TFA, gradient 5 to 87% for 3 min, retention time 1.64 min). MS (ESI) m/z 426.1 [MH]+. ¹H NMR (400 MHz, CDCl₃) δ 7.24 (t, J=7.9 Hz, 1H), 6.88 (d, J=7.7 Hz, 1H), 6.84 (s, 1H), 6.78 (dd, J=8.1, 1.9 Hz, 1H), 4.90-4.70 (m, 2H), 3.96 (t, J=6.5 Hz, 2H), 3.72 (dd, J=14.5, 9.9 Hz, 2H), 3.60 (t, J=6.3 Hz, 2H), 3.58-3.54 (m, 2H), 3.53 (t, J=6.5 Hz, 2H), 3.45 (t, J=6.4 Hz, 2H), 1.92-1.84 (m, 2H), 1.85-1.77 (m, 2H), 1.72-1.60 (m, 2H), 1.56-1.52 (m, 2H), 1.47-1.40 (m, 13H).

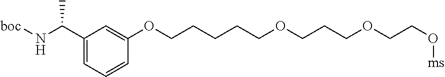

Compound 6. ((R)-2-(3-((5-(3-(1-((tert-butoxycarbonyl)amino)ethyl)phenoxy)pentyl)oxy)propoxy)ethyl methanesulfonate. A solution of compound 5 (300 mg, 0.705 mmol) in CH₂Cl₂ was mixed with 0.18 mL of DIPEA (1.058 mmol) and cooled to 0° C. 57 mg (0.846 mmol) of MsCl was added dropwise. The mixture was stirred at room temperature for 2 hours, washed with water, dried over Na₂SO₄ and evaporated. The product was used without purification. Yield 323 mg (91%)

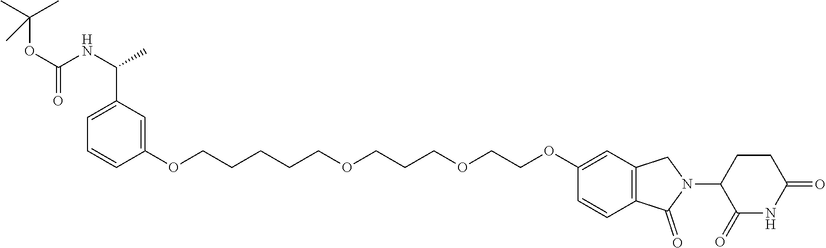 [SMILES: C[C@@H](NC(=O)OC(C)(C)C)c1cccc(OCCCCCOCCCOCCOc2ccc3c(c2)CN(C2CCC(=O)NC2=O)C3=O)c1]

Compound 7. tert-butyl ((1R)-1-(3-((5-(3-(2-((2-(2,6-dioxopiperidin-3-yl)-1-oxoisoindolin-5-yl)oxy)ethoxy)propoxy)pentyl)oxy)phenyl)ethyl)carbamate. Compound 6 (0.635 mmol, 1 eq), compound 5-OH-LNDM (0.635 mmol, 1 eq) and K₂CO₃ (88 mg, 1 eq) in dry acetonitrile (50 mL) and DMF (10 ml) were stirred for 36 h at reflux. The precipitate was filtered off, washed with acetonitrile. The solvent was evaporated to dryness. The residue was purified by silica gel flash column chromatography using ethylacetate as an eluent to afford the desired product 7. Yield 17%. LCMS (C18 column 20×2 mm, 2.5 μm, pore size 100 Å, water-acetonitrile+0.1% TFA, gradient 5 to 87% for 10 min, retention time 7.23 min). MS (ESI) m/z 669.2 [MH]+. ¹H NMR (400 MHz, DMSO-d₆) δ 10.95 (s, 1H), 7.62 (d, J=8.3 Hz, 1H), 7.46-7.14 (m, 3H), 7.05 (d, J=8.3 Hz, 1H), 6.92-6.79 (m, J=8.5 Hz, 3H), 6.74 (d, J=7.9 Hz, 1H), 5.19-5.04 (m, 1H), 4.66-4.49 (m, 1H), 4.41-4.32 (m, J=17.3 Hz, 2H), 3.91 (t, J=6.5 Hz, 2H), 3.75-3.67 (m, 2H), 3.50 (t, J=6.4 Hz, 2H), 3.44-3.36 (m, 6H), 3.00-2.82 (m, 1H), 2.70-2.52 (m, 1H), 2.47-2.28 (m, 1H), 2.07-1.92 (m, 1H), 1.78-1.61 (m, 4H), 1.57-1.46 (m, 2H), 1.46-1.30 (m, 11H), 1.27 (d, J=7.0 Hz, 3H).

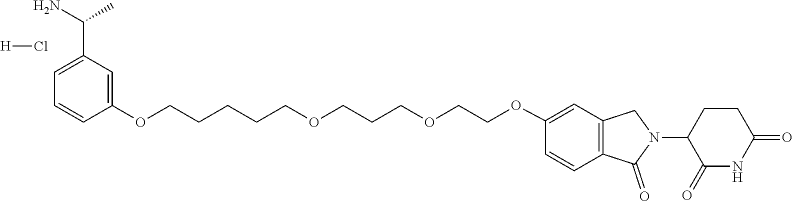

Compound 8. 3-(5-(2-(3-((5-(3-((R)-1-aminoethyl)phenoxy)pentyl)oxy)propoxy)ethoxy)-1-oxoisoindolin-2-yl)piperidine-2,6-dione hydrochloride. To an ice cold solution of compound 7 (0.31 g, 0.47 mmol) in methylene chloride (10 ml) a dioxanic 3M HCl solution (1 ml) was added. The mixture was stirred at ambient temperature overnight and then evaporated dryness. The residue was taken up with dry ether (20 ml) and the solid was filtered, washed with ether (10 ml) and dried on air. Yield of 8 was 0.28 g (99%) that was used further without purification. (C18 column 20×2 mm, 2.5 μm, pore size 100 Å, water-acetonitrile+0.1% TFA, gradient 5 to 87% for 10 min, retention time 4.55 min). MS (ESI) m/z 568.3 [MH]+.

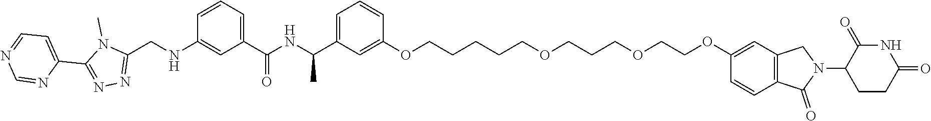 [SMILES: C[C@@H](NC(=O)c1cccc(NCc2nnc(-c3ccncn3)n2C)c1)c1cccc(OCCCCCOCCCOCCOc2ccc3c(c2)CN(C2CCC(=O)NC2=O)C3=O)c1]

Compound 10. D160. N-((1R)-1-(3-((5-(3-(2-((2-(2,6-dioxopiperidin-3-yl)-1-oxoisoindolin-5-yl)oxy)ethoxy)propoxy)pentyl)oxy)phenyl)ethyl)-3-(((4-methyl-5-(pyrimidin-4-yl)-4H-1,2,4-triazol-3-yl)methyl)amino)benzamide. To an ice cold suspension of amine hydrochloride 8 (0.1 g, 0.16 mmol) in methylene chloride (5 ml), acid 9 (0.06 g, 0.19 mmol) was added, followed by hydroxyl benzotriazole hydrate (0.032 g, 0.21 mmol) and EDCI (0.04 g, 0.21 mmol) and finally DIPEA (0.5 ml, 3.86 mmol). The reaction mixture was stirred at ambient temperature overnight. Then water (5 ml) was added and organic layer was separated. Aqueous layer was extracted with methylene chloride (5 ml). Combined organic layers were dried over MgSO₄ and evaporated. The residue was purified by HPLC in neutral conditions (YMC-Pack ODS-AQ 250×20 mml, S-10 μm, 12 nm, gradient water-acetonitrile). Yield 31%. LCMS (C18 column 20×2 mm, 2.5 μm, pore size 100 Å, water-acetonitrile+0.1% TFA, gradient 5 to 87% for 10 min, retention time 6.03 min). MS (ESI) m/z 861.0 [MH]+. ¹H NMR (400 MHz, DMSO-d₆) δ 10.95 (s, 1H), 9.33 (d, J=1.4 Hz, 1H), 8.96 (d, J=5.3 Hz, 1H), 8.55 (d, J=8.2 Hz, 1H), 8.17 (dd, J=5.2, 1.5 Hz, 1H), 7.61 (d, J=8.4 Hz, 1H), 7.18 (m, 4H), 7.10 (d, J=7.6 Hz, 1H), 7.04 (dd, J=8.5, 2.2 Hz, 1H), 6.91 (m, 3H), 6.76 (m, 1H), 6.45 (t, J=5.7 Hz, 1H), 5.07 (m, 2H), 4.57 (d, J=5.7 Hz, 2H), 4.31 (m, 2H), 4.16 (t, J=4.5 Hz, 2H), 4.07 (s, 3H), 3.92 (t, J=6.4 Hz, 2H), 3.71 (t, J=4.6 Hz, 2H), 3.50 (t, J=6.4 Hz, 2H), 3.39 (t, J=6.4 Hz, 2H), 3.34 (t, J=6.3 Hz, 2H), 2.89 (m, 1H), 2.58 (m, 1H), 2.36 (m, 1H), 1.97 (m, 1H), 1.71 (m, 4H), 1.51 (m, 2H), 1.42 (m, 5H).

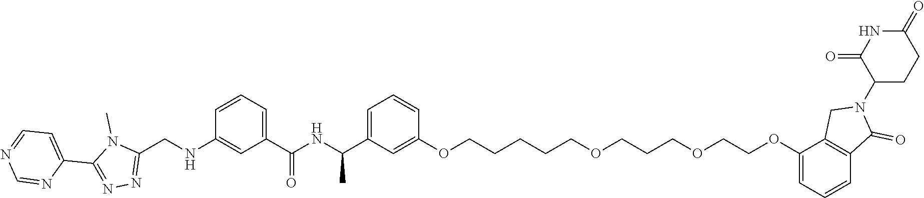 [SMILES: C[C@@H](NC(=O)c1cccc(NCc2nnc(-c3ccncn3)n2C)c1)c1cccc(OCCCCCOCCCOCCOc2cccc3c2CN(C2CCC(=O)NC2=O)C3=O)c1]

D198. N-((1R)-1-(3-((5-(3-(2-((2-(2,6-dioxopiperidin-3-yl)-1-oxoisoindolin-4-yl)oxy)ethoxy)propoxy)pentyl)oxy)phenyl)ethyl)-3-(((4-methyl-5-(pyrimidin-4-yl)-4H-1,2,4-triazol-3-yl)methyl)amino)benzamide. To an ice cold suspension of amine hydrochloride 8 (0.1 g, 0.16 mmol) in methylene chloride (5 ml), acid 9 (0.06 g, 0.19 mmol) was added, followed by hydroxyl benzotriazole hydrate (0.032 g, 0.21 mmol) and EDCI (0.04 g, 0.21 mmol) and finally DIPEA (0.5 ml, 3.86 mmol). The reaction mixture was stirred at ambient temperature overnight. Then water (5 ml) was added and organic layer was separated. Aqueous layer was extracted with methylene chloride (5 ml). Combined organic layers were dried over MgSO₄ and evaporated. The residue was purified by HPLC in neutral conditions (YMC-Pack ODS-AQ 250×20 mml, S-10 μm, 12 nm, gradient water-acetonitrile). Yield 51%. LCMS (C18 column 20×2 mm, 2.5 μm, pore size 100 Å, water-acetonitrile+0.1% TFA, gradient 5 to 87% for 10 min, retention time 5.99 min). MS (ESI) m/z 860.9 [MH]+. ¹H NMR (400 MHz, DMSO-d₆) δ 10.96 (s, 1H), 9.33 (d, J=1.4 Hz, 1H), 8.96 (d, J=5.3 Hz, 1H), 8.55 (d, J=8.2 Hz, 1H), 8.17 (dd, J=5.3, 1.4 Hz, 1H), 7.46 (t, J=7.8 Hz, 1H), 7.31 (d, J=7.4 Hz, 1H), 7.19 (m, 4H), 7.10 (dt, J=7.8, 1.3 Hz, 1H), 6.91 (m, 3H), 6.76 (m, 1H), 6.46 (t, J=5.7 Hz, 1H), 5.10 (m, 2H), 4.57 (d, J=5.6 Hz, 2H), 4.36 (d, J=17.4 Hz, 1H), 4.22 (m, 3H), 4.06 (s, 3H), 3.91 (t, J=6.4 Hz, 2H), 3.70 (m, 2H), 3.49 (t, J=6.4 Hz, 2H), 3.37 (t, J=6.4 Hz, 2H), 3.31 (m, 2H), 2.90 (m, 1H), 2.59 (m, 1H), 2.43 (m, 1H), 1.97 (m, 1H), 1.69 (m, 4H), 1.49 (m, 2H), 1.41 (m, 5H).

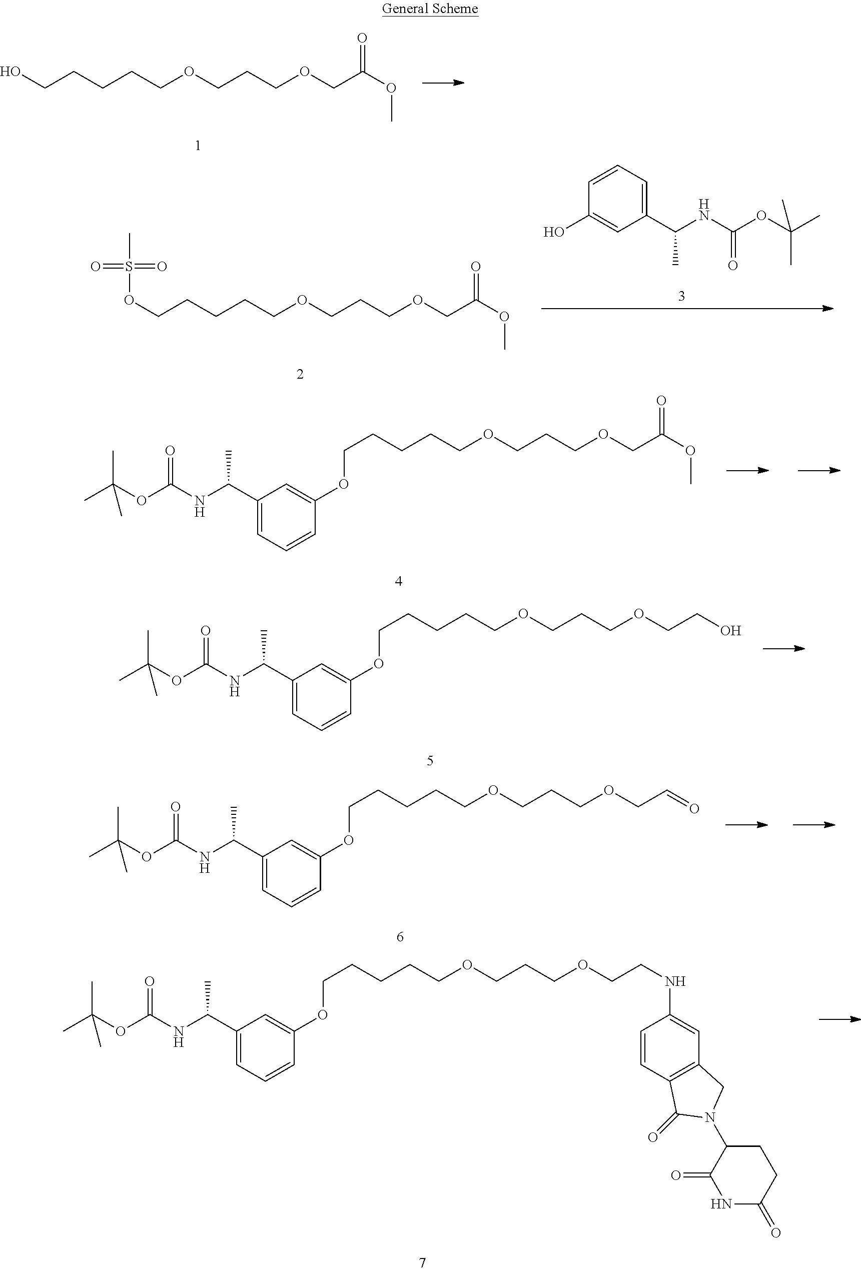

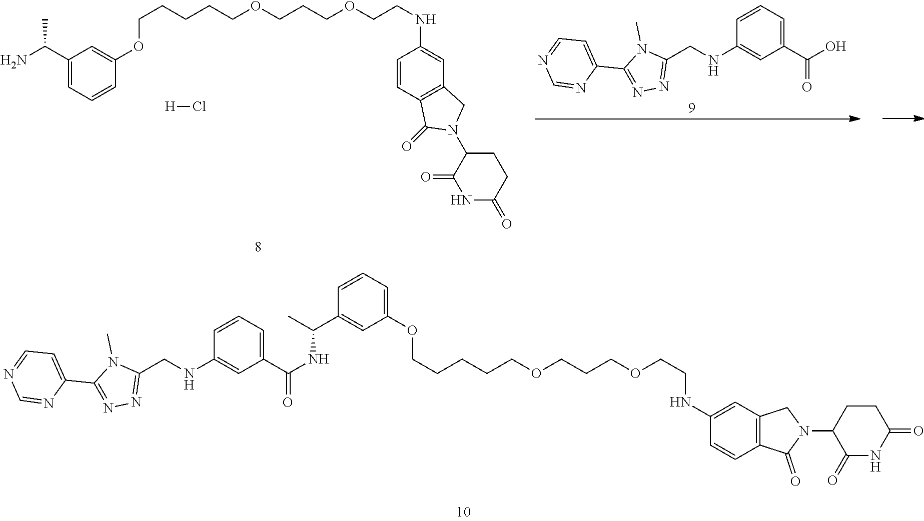 [SMILES: C[C@@H](N)c1cccc(OCCCCCOCCCOCCNc2ccc3c(c2)CN(C2CCC(=O)NC2=O)C3=O)c1 ; Cl ; Cn1c(CNc2cccc(C(=O)O)c2)nnc1-c1ccncn1 ; C[C@@H](NC(=O)c1cccc(NCc2nnc(-c3ccncn3)n2C)c1)c1cccc(OCCCCCOCCCOCCNc2ccc3c(c2)CN(C2CCC(=O)NC2=O)C3=O)c1]

### General Procedures

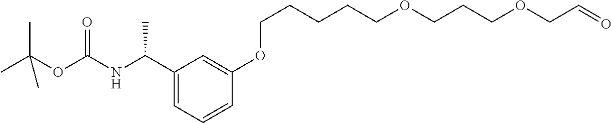 [SMILES: C[C@@H](NC(=O)OC(C)(C)C)c1cccc(OCCCCCOCCCOCC=O)c1]

Compound 6. (R)-tert-butyl 1-(3-(5-(3-(2-oxoethoxy)propoxy)pentyloxy)phenyl)ethylcarbamate. A flask was charged with 10 mL of dichloromethane and (0.1 ml, 0.145 g, 0.11 mmol) of oxalyl chloride. The solution was stirred and cooled at −50 to −60° C. as (0.12 ml, 0.132 g, 0.17 mmol) of dimethyl sulfoxide in 10 mL of dichloromethane was added dropwise at a rapid rate. After 5 min (0.3 g, 0.7 mmol) of compound 5 was added dropwise over 10 min maintaining the temperature at −50 to −60° C. After another 15 min, 0.5 mL of triethylamine (3.59 mmol) was added dropwise while keeping the temperature at or below −50° C. Stirring was continued for 5 min, after which time the mixture was allowed to warm to room temperature and 20 mL of water was added. The aqueous layer was separated and extracted with two 10-ml portions of dichloromethane. The organic layers were combined and dried over anhydrous magnesium sulfate. The filtered solution was concentrated to afford 0.29 g (97%) of compound 6 that is used further without additional purification. ¹H NMR (400 MHz, CDCl₃) δ 9.74 (s, 1H), 7.24 (t, J=7.9 Hz, 1H), 6.88 (d, J=7.5 Hz, 1H), 6.84 (s, 1H), 6.78 (dd, J=8.1, 1.8 Hz, 1H), 4.90-4.70 (m, 2H), 4.08 (s, 2H), 3.96 (t, J=6.4 Hz, 2H), 3.65 (t, J=6.3 Hz, 2H), 3.54 (t, J=6.3 Hz, 2H), 3.45 (t, J=6.6 Hz, 2H), 1.92 (p, J=6.3 Hz, 2H), 1.86-1.75 (m, 2H), 1.70-1.59 (m, 2H), 1.59-1.50 (m, 2H), 1.47-1.40 (m, 12H).

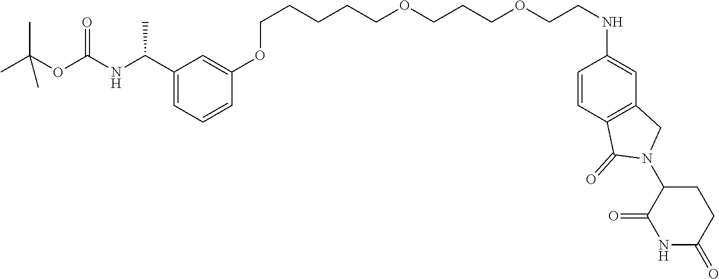 [SMILES: C[C@@H](NC(=O)OC(C)(C)C)c1cccc(OCCCCCOCCCOCCNc2ccc3c(c2)CN(C2CCC(=O)NC2=O)C3=O)c1]

Compound 7. tert-butyl (1R)-1-(3-(5-(3-(2-(2-(2,6-dioxopiperidin-3-yl)-1-oxoisoindolin-5-ylamino)ethoxy) propoxy)pentyloxy)phenyl)ethylcarbamate. To a solution of 5-lenalidomide (0.18 g, 0.69 mmol, 1.0 eq) and compound 6 (0.29 g, 0.68 mmol, 1.0 eq) in 50 mL of DCE was added acetic acid (0.24 mL, 4.18 mmol, 6.0 eq) and sodium triacetoxyborohydride (0.6 g, 2.83 mmol, 4.0 eq). The suspended solution was stirred at room temperature for 12 h., saturated NaHCO₃ aqueous solution added and extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 7 (0.24 g, 54%). LCMS (C18 column 20×2 mm, 2.5 μm, pore size 100 Å, water-acetonitrile+0.1% TFA, gradient 5 to 87% for 3 min, retention time 1.64 min). MS (ESI) m/z 667.5 [MH]+. ¹H NMR (400 MHz, CDCl₃) δ 8.21 (s, 1H), 7.66 (d, J=8.4 Hz, 1H), 7.22 (t, J=7.8 Hz, 1H), 6.87 (d, J=7.9 Hz, 1H), 6.84 (s, 1H), 6.77 (d, J=8.4 Hz, 1H), 6.67 (d, J=8.4 Hz, 1H), 6.59 (s, 1H), 5.55 (d, J=4.2 Hz, 1H), 5.26-5.10 (m, 1H), 4.86 (s, 1H), 4.75 (s, 1H), 4.38 (dd, J=15.6, 4.4 Hz, 1H), 4.22 (dd, J=15.6, 5.5 Hz, 1H), 4.06 (td, J=8.0, 5.3 Hz, 1H), 3.95 (t, J=6.4 Hz, 2H), 3.86 (dd, J=15.0, 6.9 Hz, 1H), 3.67 (t, J=5.1 Hz, 1H), 3.58 (t, J=6.3 Hz, 2H), 3.51 (dd, J=13.4, 7.2 Hz, 2H), 3.44 (t, J=6.5 Hz, 2H), 3.34 (t, J=5.2 Hz, 1H), 3.23 (q, J=7.1 Hz, 1H), 2.96-2.72 (m, 3H), 2.38-2.23 (m, 1H), 2.23-2.11 (m, 1H), 2.00-1.83 (m, 3H), 1.83-1.73 (m, 2H), 1.69-1.58 (m, 2H), 1.57-1.47 (m, 2H), 1.43 (s, 9H).

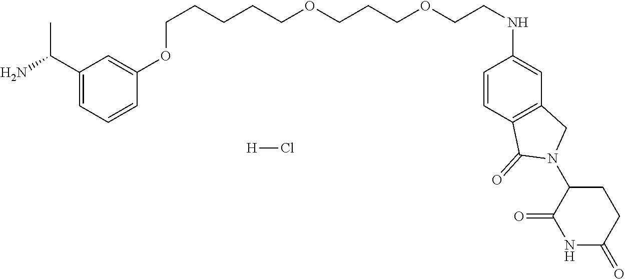 [SMILES: C[C@@H](N)c1cccc(OCCCCCOCCCOCCNc2ccc3c(c2)CN(C2CCC(=O)NC2=O)C3=O)c1 ; Cl]

Compound 8. 3-(5-(2-(3-(5-(3-((R)-1-aminoethyl)phenoxy)pentyloxy)propoxy)ethylamino)-1-oxoisoindolin-2-yl)piperidine-2,6-dione hydrochloride. To an ice cold solution of compound 7 (0.24 g, 0.37 mmol) in methylene chloride (10 ml) a dioxanic 3M HCl solution (1 ml) was added. The mixture was stirred at ambient temperature overnight and then evaporated dryness. The residue was taken up with dry ether (20 ml) and the solid was filtered, washed with ether (10 ml) and dried on air. Yield 0.22 g (99%) that was used further without purification. LCMS (C18 column 20×2 mm, 2.5 μm, pore size 100 Å, water-acetonitrile+0.1% TFA, gradient 5 to 87% for 3 min, retention time 1.14 min). MS (ESI) m/z 567.5 [MH]+.

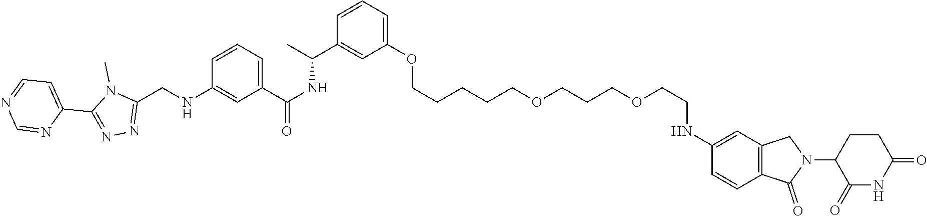 [SMILES: C[C@@H](NC(=O)c1cccc(NCc2nnc(-c3ccncn3)n2C)c1)c1cccc(OCCCCCOCCCOCCNc2ccc3c(c2)CN(C2CCC(=O)NC2=O)C3=O)c1]

Compound 10. D171. N-((1R)-1-(3-(5-(3-(2-(2-(2,6-dioxopiperidin-3-yl)-1-oxoisoindolin-5-ylamino) ethoxy)propoxy)pentyloxy)phenyl)ethyl)-3-((4-methyl-5-(pyrimidin-4-yl)-4H-1,2,4-triazol-3-yl)methylamino)benzamide. To an ice cold suspension of amine hydrochloride 8 (0.1 g, 0.16 mmol) in methylene chloride (5 ml), acid 9 (0.06 g, 0.19 mmol) was added, followed by hydroxyl benzotriazole hydrate (0.032 g, 0.21 mmol) and EDCI (0.04 g, 0.21 mmol) and finally DIPEA (0.5 ml, 3.86 mmol). The reaction mixture was stirred at ambient temperature overnight. Then water (5 ml) was added and organic layer was separated. Aqueous layer was extracted with methylene chloride (5 ml). Combined organic layers were dried over MgSO₄ and evaporated. The residue was purified by HPLC in neutral conditions (YMC-Pack ODS-AQ 250×20 mml, S-10 μm, 12 nm, gradient water-acetonitrile). Yield 0.045 g (31%). LCMS (C18 column 20×2 mm, 2.5 μm, pore size 100 Å, water-acetonitrile+0.1% TFA, gradient 5 to 87% for 10 min, retention time 5.79 min). MS (ESI) m/z 859.9 [MH]+. ¹H NMR (400 MHz, DMSO-d₆) δ 10.91 (s, 1H), 9.33 (d, J=1.3 Hz, 1H), 8.97 (d, J=5.3 Hz, 1H), 8.56 (d, J=8.2 Hz, 1H), 8.17 (dd, J=5.3, 1.3 Hz, 1H), 7.37 (d, J=8.3 Hz, 1H), 7.19 (q, J=8.4 Hz, 3H), 7.10 (d, J=7.7 Hz, 1H), 6.96-6.86 (m, 3H), 6.79-6.71 (m, 1H), 6.71-6.63 (m, 2H), 6.46 (t, J=5.6 Hz, 1H), 6.34 (t, J=5.6 Hz, 1H), 5.10 (dd, J=14.5, 7.3 Hz, 1H), 5.00 (dd, J=13.4, 5.0 Hz, 1H), 4.57 (d, J=5.6 Hz, 2H), 4.25 (d, J=16.7 Hz, 1H), 4.12 (dd, J=16.3, 7.9 Hz, 1H), 4.07 (s, 3H), 3.92 (t, J=6.4 Hz, 2H), 3.52 (t, J=5.6 Hz, 2H), 3.45 (t, J=6.4 Hz, 2H), 3.38 (t, J=6.4 Hz, 2H), 3.32 (t, J=6.4 Hz, 2H), 3.24 (dd, J=11.0, 5.5 Hz, 2H), 2.94-2.81 (m, 1H), 2.57 (s, 1H), 2.32 (td, J=13.3, 8.9 Hz, 1H), 1.98-1.88 (m, 1H), 1.77-1.62 (m, 4H), 1.50 (dd, J=13.8, 6.6 Hz, 2H), 1.42 (t, J=7.2 Hz, 5H).

### Example 27: Preparation of Compound D150, D155, D167, D197, D196, and D192

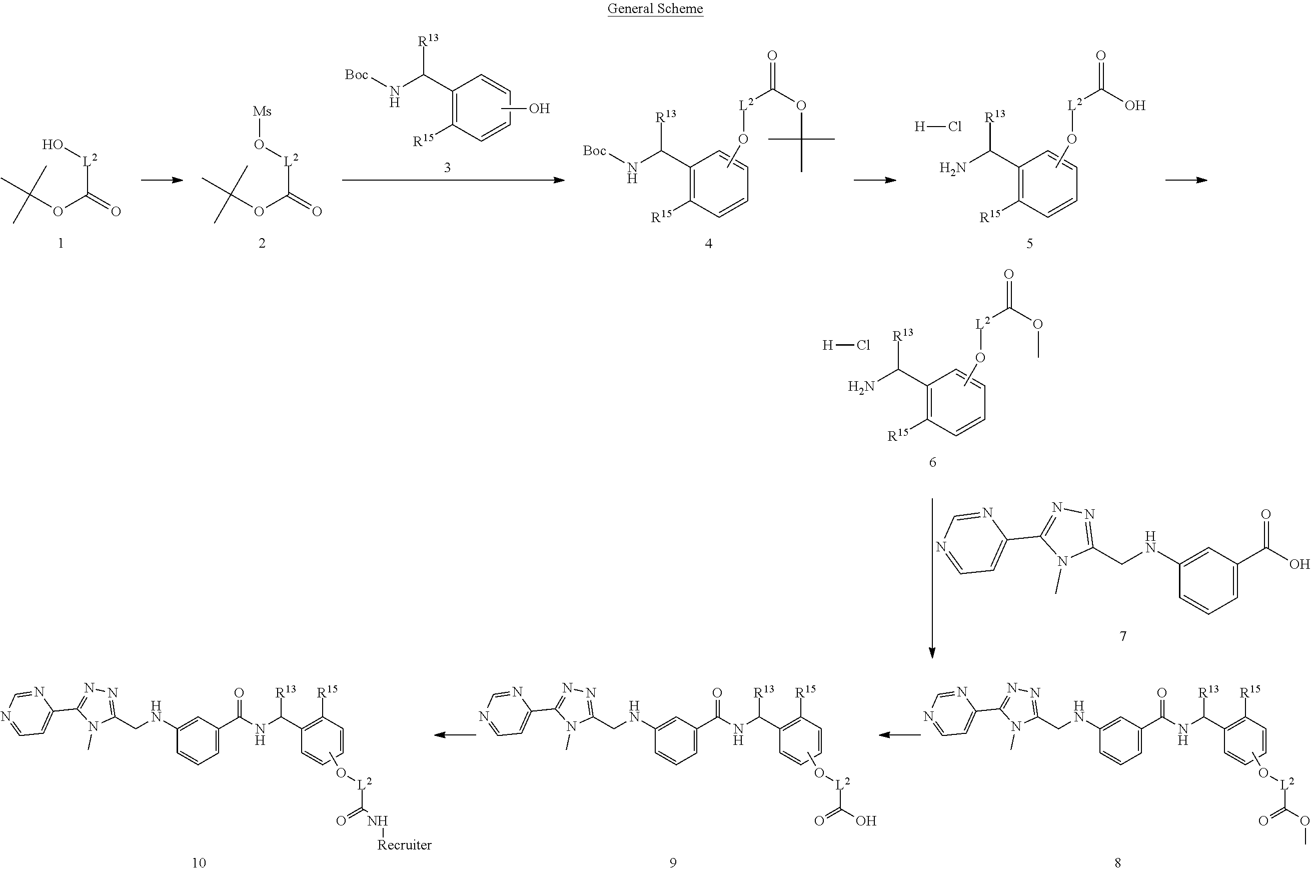

### General Procedures

Compound 2. A solution of compound 1 (2.9 mmol, 1 eq) and diisopropylamine (0.56 g, 0.75 ml, 4.35 mmol, 1.5 eq) in dry DCM (50 mL) was cooled to 0° C., and mesyl chloride (0.40 g, 0.27 ml, 3.48 mmol, 1.2 eq) was added. The mixture was warmed to 0° C. over 1 h period, stirred for 1 h. A progress of the reaction was monitored by TLC. The mixture was washed by water (3*20 ml), dried over Na₂SO₄, and evaporated. The product immediately was used in the next step without additional purification.

Compound 4. Compound 3 (2.5 mmol, 1 eq), compound 2 (2.75 mmol, 1.1 eq), K₂CO₃ (5.0 mmol, 2 eq) in dry acetonitrile (50 mL) were stirred for 12 h at reflux. The precipitate was filtered off, washed with acetonitrile. The solvent was evaporated to dryness to give the product. The product 4 was used in the next step without additional purification.

Compound 5. A solution of compound 4 (2.5 mmol, 1 eq) in dry DCM (50 mL) with 12.5 ml 3 M HCl in dioxane (5 eq) were stirred for 24 h at r.t. The mixture was evaporated to dryness to give the crude product 5 which was used in the next step without additional purification.

Compound 6. To a solution of compound 5 (2.5 mmol, 1 eq) in dry MeOH (20 mL) was added dropwise SOCl₂ (0.36 ml, 5.0 mmol, 2 eq). The mixture was stirred for 12 h at r.t. and then evaporated to dryness to give the crude product 5 which no need additional purification.

Compound 8. Compound 6 (1.2 mmol, 1 eq), compound 7 (1.2 mmol, 1 eq), HOBt (1.2 mmol, 1 eq), EDCI (1.32 mmol, 1.1 eq) and DIPEA (4.8 mmol, 4 eq) in dry DCM (30 ml) were stirred for 12 h at r.t. The reaction mixture was washed with water (3*10 ml), brine (2×10 mL), dried on activated Na₂SO₄, and concentrated in a rotary evaporator under reduced pressure at ambient temperature. The residue was purified by silica gel flash column chromatography using chloroform and methanol as an eluent to afford the desired product 8.

Compound 9. To a solution of compound 8 (1.2 mmol, 1 eq) in MeOH (20 mL) was added a solution KOH (3.6 mmol, 3 eq) in 3 ml H₂O. The mixture was stirred for 12-36 h at r.t. and then MeOH evaporated. To the residue was added 15 ml of H₂O and the mixture was acidified to pH=5 with 1N aq. HCl and aqueous phase was extracted with 4*15 ml of DCM. Combined organic phases were washed with brine, dried over Na₂SO₄, and evaporated. The product was used in the next step without additional purification.

Compound 10. Compound 9 (0.1 mmol, 1 eq), recruiter (0.1 mmol, 1 eq), TBTU (0.2 mmol, 2 eq) and DIPEA (0.4 mmol, 4 eq) in dry pyridine (3 ml) were stirred for 12 h at r.t. The mixture was evaporated to dryness. The residue was purified by HPLC in neutral conditions (YMC-Pack ODS-AQ 250×20 mml, S-10 μm, 12 nm, gradient water-acetonitrile)

### Syntheses According to the General Procedures

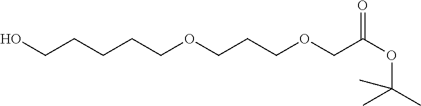 [SMILES: CC(C)(C)OC(=O)COCCCOCCCCCO]

Compound 1. Tert-butyl 2-(3-((5-hydroxypentyl)oxy)propoxy)acetate was prepared according to the procedures ACS Med. Chem. Lett. 2020, 11, 8, 1539-1547.

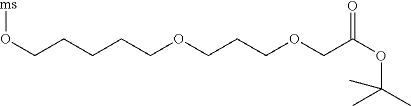

Compound 2. Tert-butyl 3,9,13-trioxa-2-thiapentadecan-15-oate 2,2-dioxide. Colorless oil. Yield 95%. The product was used in the next step immediately.

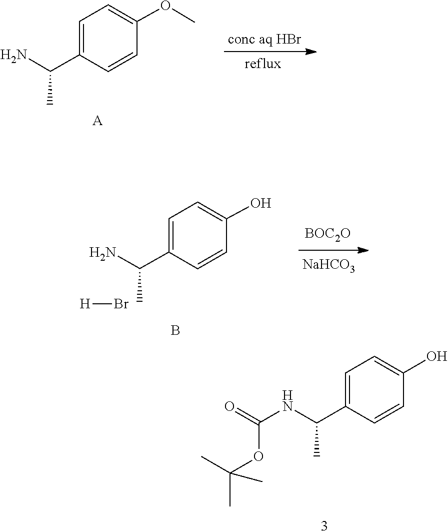

Compound 3. (S)-tert-butyl (1-(4-hydroxyphenyl)ethyl)carbamate. A solution of (S)-1-(4-methoxyphenyl)ethanamine (5.0 g, 33.07 mmol) in 30 mL HBr (48% w/w in H₂O) was stirred at 100° C. for 12 h. The reaction mixture was cooled, the solution was decanted and concentrated under vacuum to afford 71% product B ((S)-4-(1-aminoethyl)phenol hydrobromide), which was used directly in the next step without further purification. To a solution of (S)-4-(1-aminoethyl)phenol hydrobromide (7.2 g, 33.06 mmol) in 100 ml of water at r.t. were added 100 ml of 1,4-dioxane, sodium hydrogen carbonate (13.9 g, 165.34 mmol, 5 eq) and di-tert-butyl dicarbonate (7.4 g, 33.73 mmol, 1.02 eq) and the resulting mixture was stirred at rt for 12 h. The reaction mixture was extracted with 3*100 ml of EtOAc. Combined organic phases were washed with brine, dried over Na₂SO₄ and evaporated. The residue was purified by silica gel flash column chromatography using chloroform and methanol as an eluent to afford 3. Yield 48%. ¹H NMR (400 MHz, DMSO-d₆) δ 9.18 (s, 1H), 7.19 (d, J=8.2 Hz, 1H), 7.08 (d, J=8.4 Hz, 2H), 6.68 (d, J=8.4 Hz, 2H), 4.56-4.46 (m, 1H), 1.35 (s, 9H), 1.26 (d, J=7.1 Hz, 3H).

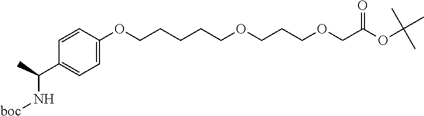

Compound 4. (S)-tert-butyl 2-(3-((5-(4-(1-((tert-butoxycarbonyl)amino)ethyl)phenoxy) pentyl)oxy)propoxy)acetate. Yellowish oil. Yield 99%. LCMS (C18 column 20×2 mm, 2.5 μm, pore size 100 Å, water-acetonitrile+0.1% TFA, gradient 5 to 87% for 3 min, retention time 2.08 min). MS (ESI) m/z 496.9 [MH]⁺.

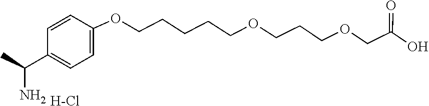

Compound 5. (S)-2-(3-((5-(4-(1-aminoethyl)phenoxy)pentyl)oxy)propoxy)acetic acid hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.00 min). MS (ESI) m/z 340.4 [MH]⁺.

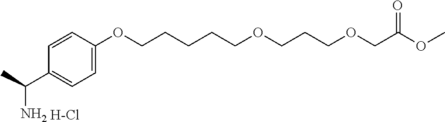

Compound 6. (S)-methyl 2-(3-((5-(4-(1-aminoethyl)phenoxy)pentyl)oxy)propoxy)acetate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.21 min). MS (ESI) m/z 354.3 [MH]⁺.

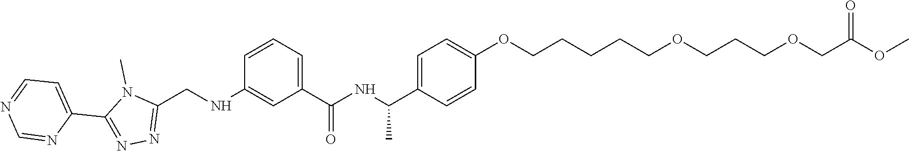 [SMILES: COC(=O)COCCCOCCCCCOc1ccc([C@H](C)NC(=O)c2cccc(NCc3nnc(-c4ccncn4)n3C)c2)cc1]

Compound 8. (S)-methyl 2-(3-((4-(3-(1-(3-(((4-methyl-5-(pyrimidin-4-yl)-4H-1,2,4-triazol-3-yl)methyl)amino)benzamido)ethyl)phenoxy)pentyl)oxy)propoxy)acetate. Light brown solid. Yield 95%. LCMS (C18 column 100×4.6 mm, 5.0 μm, pore size 100 Å, water-acetonitrile+0.1% TFA, gradient 5 to 87% for 10 min, retention time 6.09 min). MS (ESI) m/z 646.6 [MH]⁺.

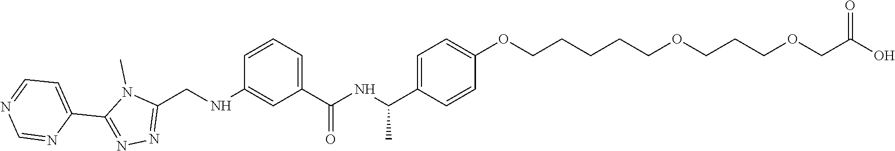 [SMILES: C[C@H](NC(=O)c1cccc(NCc2nnc(-c3ccncn3)n2C)c1)c1ccc(OCCCCCOCCCOCC(=O)O)cc1]

Compound 9. (S)-2-(3-((5-(4-(1-(3-(((4-methyl-5-(pyrimidin-4-yl)-4H-1,2,4-triazol-3-yl)methyl)amino)benzamido)ethyl)phenoxy)pentyl)oxy)propoxy)acetic acid. Light brown solid. Yield 74%. LCMS (C18 column 100×4.6 mm, 5.0 μm, pore size 100 Å, water-acetonitrile+0.1% TFA, gradient 5 to 87% for 10 min, retention time 5.48 min). MS (ESI) m/z 632.5 [MH]⁺.

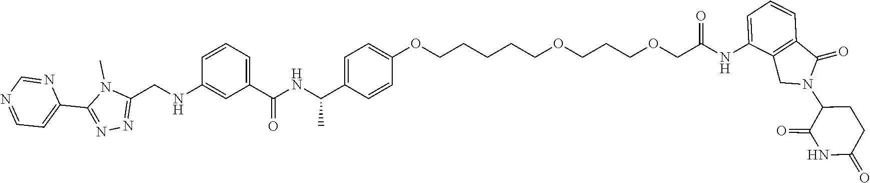 [SMILES: C[C@H](NC(=O)c1cccc(NCc2nnc(-c3ccncn3)n2C)c1)c1ccc(OCCCCCOCCCOCC(=O)Nc2cccc3c2CN(C2CCC(=O)NC2=O)C3=O)cc1]

Compound 10. D150. N-((1S)-1-(4-((5-(3-(2-((2-(2,6-dioxopiperidin-3-yl)-1-oxoisoindolin-4-yl)amino)-2-oxoethoxy)propoxy)pentyl)oxy)phenyl)ethyl)-3-(((4-methyl-5-(pyrimidin-4-yl)-4H-1,2,4-triazol-3-yl)methyl)amino)benzamide. Colorless solid. Yield 38%. LCMS (C18 column 100×4.6 mm, 5.0 μm, pore size 100 Å, water-acetonitrile+0.1% TFA, gradient 5 to 87% for 10 min, retention time 5.60 min). MS (ESI) m/z 873.4 [MH]⁺. ¹H NMR (400 MHz, DMSO-d₆) δ 10.99 (s, 1H), 9.69 (s, 1H), 9.33 (d, J=1.4 Hz, 1H), 8.97 (d, J=5.2 Hz, 1H), 8.51 (d, J=8.2 Hz, 1H), 8.17 (dd, J=5.3, 1.4 Hz, 1H), 7.73 (dd, J=7.8, 1.2 Hz, 1H), 7.55 (dd, J=7.6, 1.2 Hz, 1H), 7.50 (t, J=7.6 Hz, 1H), 7.27 (d, J=8.6 Hz, 2H), 7.17 (m, 2H), 7.09 (m, 1H), 6.89 (m, 1H), 6.84 (d, J=8.8 Hz, 2H), 6.44 (t, J=5.7 Hz, 1H), 5.13 (dd, J=13.3, 5.1 Hz, 1H), 5.08 (m, 1H), 4.57 (d, J=5.6 Hz, 2H), 4.35 (m, 2H), 4.07 (s, 2H), 4.07 (s, 3H), 3.90 (t, J=6.4 Hz, 2H), 3.58 (t, J=6.4 Hz, 2H), 3.46 (t, J=6.4 Hz, 2H), 3.36 (t, J=6.4 Hz, 2H), 2.89 (m, 1H), 2.59 (m, 1H), 2.35 (m, 1H), 2.00 (m, 1H), 1.81 (p, J=6.4 Hz, 2H), 1.67 (p, J=6.5 Hz, 2H), 1.52 (m, 2H), 1.42 (m, 5H).

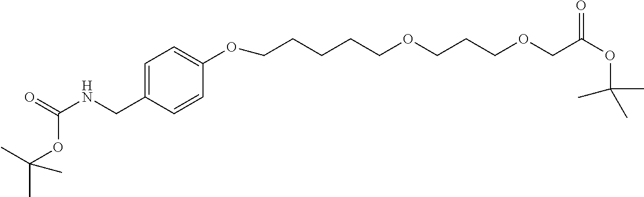 [SMILES: CC(C)(C)OC(=O)COCCCOCCCCCOc1ccc(CNC(=O)OC(C)(C)C)cc1]

Compound 4. tert-butyl 2-(3-(5-(4-((tert-butoxycarbonylamino)methyl)phenoxy)pentyloxy) propoxy)acetate. Yield 75%. LCMS (C18 column 20×2 mm, 2.5 μm, pore size 100 Å, water-acetonitrile+0.1% TFA, gradient 5 to 87% for 3 min, retention time 1.92 min). MS (ESI) m/z 482.4[MH]+. ¹H NMR (400 MHz, CDCl₃) δ 7.26 (t, J=6.1 Hz, 1H), 7.11 (d, J=8.6 Hz, 2H), 6.86 (d, J=8.6 Hz, 2H), 4.02 (d, J=14.0 Hz, 2H), 3.92 (s, 2H), 3.47 (t, J=6.4 Hz, 2H), 3.40 (t, J=6.4 Hz, 2H), 3.36 (t, J=6.4 Hz, 2H), 1.75-1.66 (m, 4H), 1.57-1.50 (m, 2H), 1.46-1.33 (m, 20H).

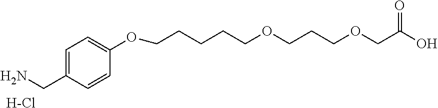

Compound 5. 2-(3-(5-(4-(aminomethyl)phenoxy)pentyloxy)propoxy)acetic acid hydrochloride. Yield 99%. LCMS (C18 column 20×2 mm, 2.5 μm, pore size 100 Å, water-acetonitrile+0.1% TFA, gradient 5 to 87% for 3 min, retention time 1.03 min). MS (ESI) m/z 326.4[MH]+.

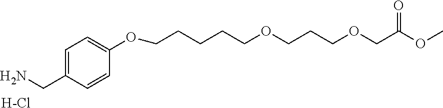

Compound 6. Methyl 2-(3-(5-(4-(aminomethyl)phenoxy)pentyloxy)propoxy)acetate hydrochloride. Yield 99%. LCMS (C18 column 20×2 mm, 2.5 μm, pore size 100 Å, water-acetonitrile+0.1% TFA, gradient 5 to 87% for 3 min, retention time 1.11 min). MS (ESI) m/z 340.4[MH]+.

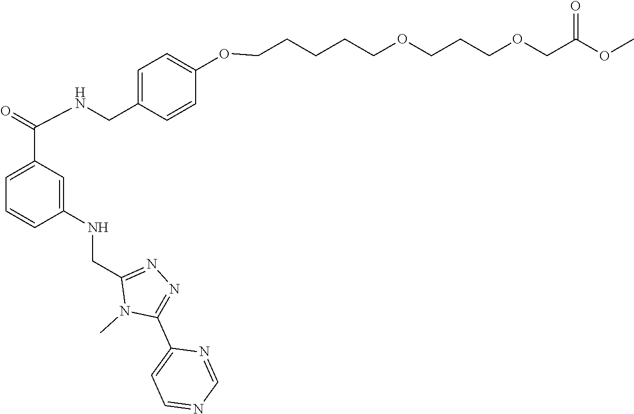 [SMILES: COC(=O)COCCCOCCCCCOc1ccc(CNC(=O)c2cccc(NCc3nnc(-c4ccncn4)n3C)c2)cc1]

Compound 8. 2-(3-((5-(4-((3-(((4-methyl-5-(pyrimidin-4-yl)-4H-1,2,4-triazol-3-yl)methyl)amino) benzamido)methyl)phenoxy)pentyl)oxy)propoxy)acetate. Yield 77%. LCMS (C18 column 20×2 mm, 2.5 μm, pore size 100 Å, water-acetonitrile+0.1% TFA, gradient 5 to 87% for 3 min, retention time 1.45 min). MS (ESI) m/z 632.8[MH]+.

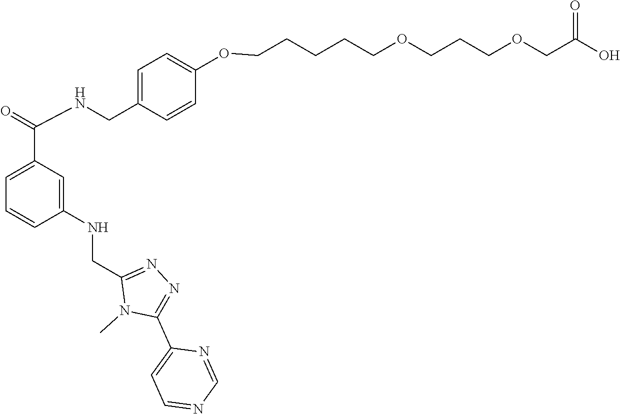 [SMILES: Cn1c(CNc2cccc(C(=O)NCc3ccc(OCCCCCOCCCOCC(=O)O)cc3)c2)nnc1-c1ccncn1]

Compound 9. 2-(3-(5-(4-((3-((4-methyl-5-(pyrimidin-4-yl)-4H-1,2,4-triazol-3-yl)methylamino)benzamido)methyl)phenoxy)pentyloxy)propoxy)acetic acid. Yield 71%. LCMS (C18 column 20×2 mm, 2.5 μm, pore size 100 Å, water-acetonitrile+0.1% TFA, gradient 5 to 87% for 3 min, retention time 1.30 min). MS (ESI) m/z 618.5[MH]+.

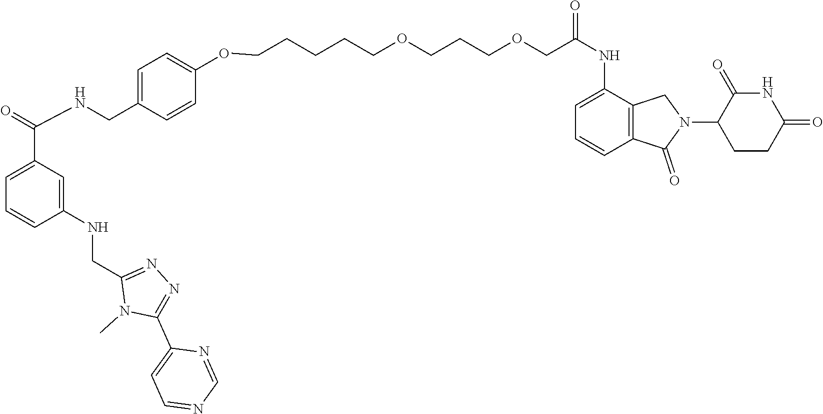 [SMILES: Cn1c(CNc2cccc(C(=O)NCc3ccc(OCCCCCOCCCOCC(=O)Nc4cccc5c4CN(C4CCC(=O)NC4=O)C5=O)cc3)c2)nnc1-c1ccncn1]

D167. N-(4-(5-(3-(2-(2-(2,6-dioxopiperidin-3-yl)-1-oxoisoindolin-4-ylamino)-2-oxoethoxy)propoxy)pentyloxy)benzyl)-3-((4-methyl-5-(pyrimidin-4-yl)-4H-1,2,4-triazol-3-yl)methylamino)benzamide. LCMS (C18 column 100×4.6 mm, 5.0 μm, pore size 100 Å, water-acetonitrile+0.1% TFA, gradient 5 to 87% for 10 min, retention time 5.45 min). MS (ESI) m/z 859.9[MH]+. ¹H NMR (400 MHz, DMSO-d₆) δ 11.00 (s, 1H), 9.69 (s, 1H), 9.33 (d, J=1.3 Hz, 1H), 8.86 (d, J=5.3 Hz, 1H), 8.77 (t, J=5.8 Hz, 1H), 8.17 (dd, J=5.3, 1.3 Hz, 1H), 7.72 (d, J=8 Hz, 1H), 7.56-7.48 (m, 2H), 7.22-7.15 (m, 4H), 7.09 (d, J=7.7 Hz, 1H), 6.90-6.84 (m, 3H), 6.46 (t, J=5.8 Hz, 1H), 5.16 (dd, J=13.2, 5.0 Hz, 1H), 4.56 (d, J=5.6 Hz, 2H), 4.42-31 (m, 4H), 4.07 (s, 5H), 3.89 (t, J=6.4 Hz, 2H), 3.58 (t, J=6.4 Hz, 2H), 3.46 (t, J=6.4 Hz, 2H), 3.36 (t, J=6.4 Hz, 2H), 2.95-2.86 (m, 1H), 2.67-2.56 (m, 1H), 2.41-2.30 (m, 1H), 2.03-1.97 (m, 1H), 1.84-1.78 (m, 2H), 1.70-1.63 (m, 2H), 1.55-1.48 (m, 2H), 1.44-1.36 (m, 2H).

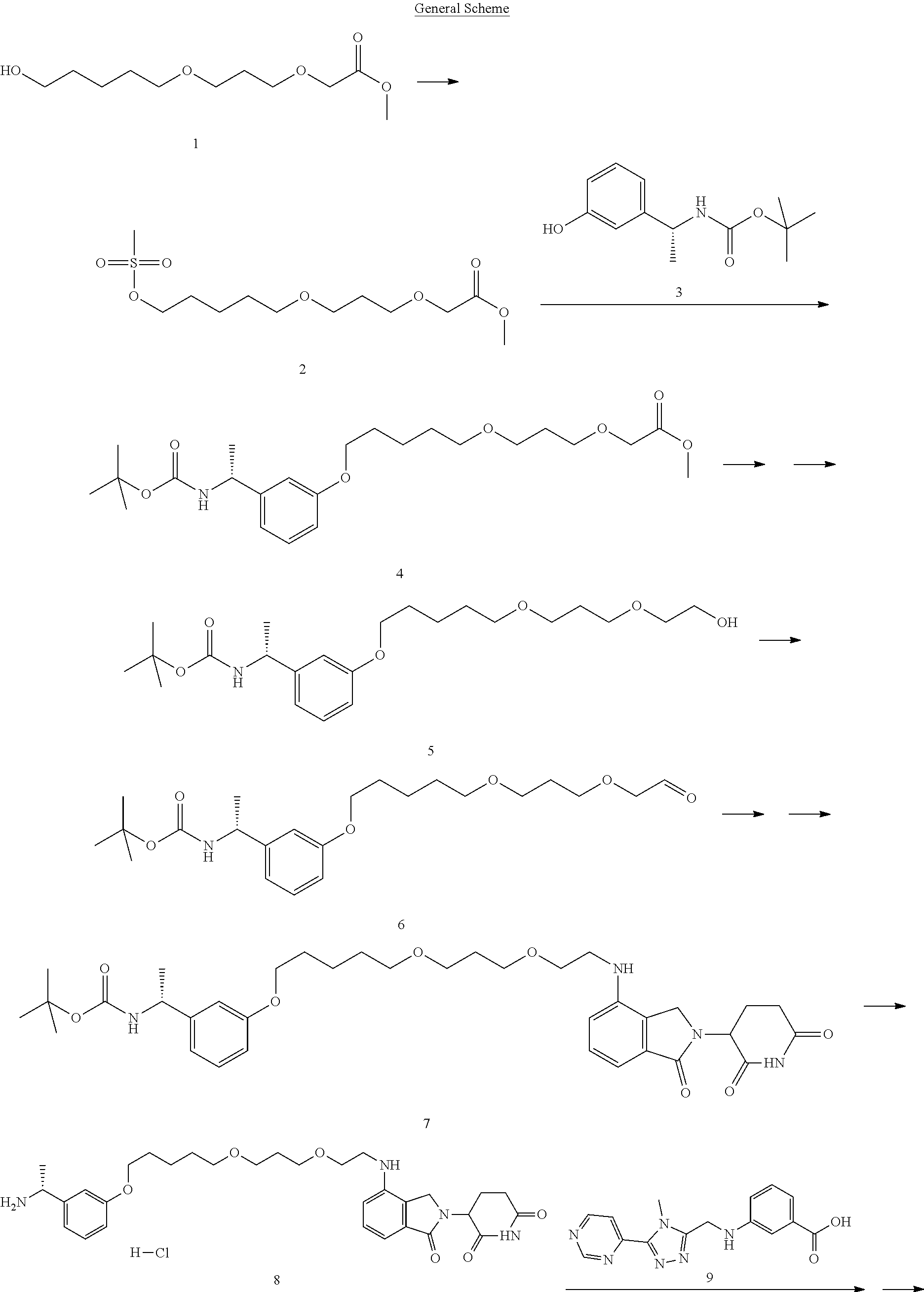

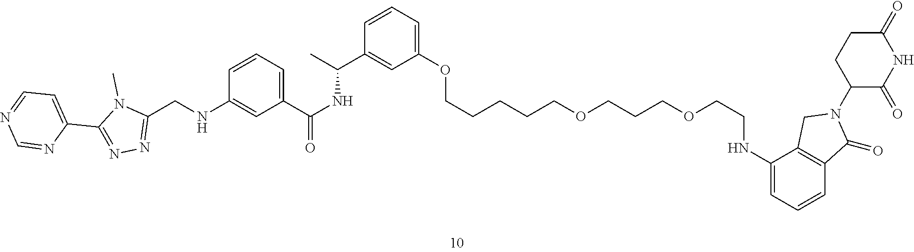 [SMILES: C[C@@H](NC(=O)c1cccc(NCc2nnc(-c3ccncn3)n2C)c1)c1cccc(OCCCCCOCCCOCCNc2cccc3c2CN(C2CCC(=O)NC2=O)C3=O)c1]

### General Procedures

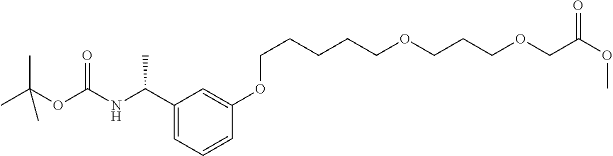 [SMILES: COC(=O)COCCCOCCCCCOc1cccc([C@@H](C)NC(=O)OC(C)(C)C)c1]

Compound 4. (R)-methyl 2-(3-(5-(3-(1-(tert-butoxycarbonylamino)ethyl)phenoxy)pentyloxy) propoxy) acetate. Compound 3 (5.18 mmol, 1 eq), compound 2 (5.37 mmol, 1.03 eq) and K₂CO₃ (1.79 g, 12.97 mmol, 2.5 eq) in dry acetonitrile (50 mL) were stirred for 12 h at reflux. The solvent was evaporated to dryness. The residue was partitioned between methylene chloride (10 ml) and water (10 ml). Organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 2.3 g (97%). LCMS (C18 column 20×2 mm, 2.5 μm, pore size 100 Å, water-acetonitrile+0.1% TFA, gradient 5 to 87% for 3 min, retention time 1.74 min). MS (ESI) m/z 454.3 [MH]+. ¹H NMR (400 MHz, CDCl₃) δ 7.23 (t, J=7.9 Hz, 1H), 6.88 (d, J=7.6 Hz, 1H), 6.84 (s, 1H), 6.78 (dd, J=8.0, 2.0 Hz, 1H), 4.90-4.70 (m, 2H), 4.10 (s, 2H), 3.96 (t, J=6.4 Hz, 2H), 3.76 (s, 3H), 3.63 (t, J=6.4 Hz, 2H), 3.53 (t, J=6.3 Hz, 2H), 3.45 (t, J=6.5 Hz, 2H), 1.90 (p, J=6.3 Hz, 2H), 1.86-1.75 (m, 2H), 1.71-1.59 (m, 2H), 1.59-1.48 (m, 2H), 1.47-1.40 (m, 12H).

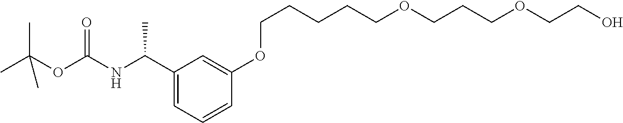 [SMILES: C[C@@H](NC(=O)OC(C)(C)C)c1cccc(OCCCCCOCCCOCCO)c1]

Compound 5. (R)-tert-butyl 1-(3-(5-(3-(2-hydroxyethoxy)propoxy)pentyloxy)phenyl)ethyl carbamate. A mixture of LiBH₄ (0.176 g, 8.0 mmol), compound 4 (2.3 g, 5.0 mmol), methanol (0.33 mL, 8.1 mmol), and ether (10 mL) was refluxed for 15 min. The reaction was quenched with aqueous citric acid (2 g in 10 ml water) with ice-cooling. The mixture was diluted with water and was extracted with dichloromethane. The extract was dried over Na₂SO₄ and the solvent was evaporated under reduced pressure. Purification by silica gel (chloroform/methanol=100:1 as developing solvent) afforded compound 5 (2.07 g, 95%). LCMS (C18 column 20×2 mm, 2.5 μm, pore size 100 Å, water-acetonitrile+0.1% TFA, gradient 5 to 87% for 3 min, retention time 1.64 min). MS (ESI) m/z 426.1 [MH]+. ¹H NMR (400 MHz, CDCl₃) δ 7.24 (t, J=7.9 Hz, 1H), 6.88 (d, J=7.7 Hz, 1H), 6.84 (s, 1H), 6.78 (dd, J=8.1, 1.9 Hz, 1H), 4.90-4.70 (m, 2H), 3.96 (t, J=6.5 Hz, 2H), 3.72 (dd, J=14.5, 9.9 Hz, 2H), 3.60 (t, J=6.3 Hz, 2H), 3.58-3.54 (m, 2H), 3.53 (t, J=6.5 Hz, 2H), 3.45 (t, J=6.4 Hz, 2H), 1.92-1.84 (m, 2H), 1.85-1.77 (m, 2H), 1.72-1.60 (m, 2H), 1.56-1.52 (m, 2H), 1.47-1.40 (m, 13H).

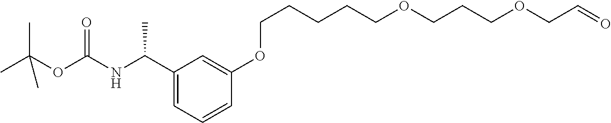 [SMILES: C[C@@H](NC(=O)OC(C)(C)C)c1cccc(OCCCCCOCCCOCC=O)c1]

Compound 6. (R)-tert-butyl 1-(3-(5-(3-(2-oxoethoxy)propoxy)pentyloxy)phenyl)ethylcarbamate. A flask was charged with 10 mL of dichloromethane and (0.1 ml, 0.145 g, 0.11 mmol) of oxalyl chloride. The solution was stirred and cooled at −50 to −60° C. as (0.12 ml, 0.132 g, 0.17 mmol) of dimethyl sulfoxide in 10 mL of dichloromethane was added dropwise at a rapid rate. After 5 min (0.3 g, 0.7 mmol) of compound 5 was added dropwise over 10 min maintaining the temperature at −50 to −60° C. After another 15 min, 0.5 mL of triethylamine (3.59 mmol) was added dropwise while keeping the temperature at or below −50° C. Stirring was continued for 5 min, after which time the mixture was allowed to warm to room temperature and 20 mL of water was added. The aqueous layer was separated and extracted with two 10-ml portions of dichloromethane. The organic layers were combined and dried over anhydrous magnesium sulfate. The filtered solution was concentrated to afford 0.29 g (97%) of compound 6 that is used further without additional purification. ¹H NMR (400 MHz, CDCl₃) δ 9.74 (s, 1H), 7.24 (t, J=7.9 Hz, 1H), 6.88 (d, J=7.5 Hz, 1H), 6.84 (s, 1H), 6.78 (dd, J=8.1, 1.8 Hz, 1H), 4.90-4.70 (m, 2H), 4.08 (s, 2H), 3.96 (t, J=6.4 Hz, 2H), 3.65 (t, J=6.3 Hz, 2H), 3.54 (t, J=6.3 Hz, 2H), 3.45 (t, J=6.6 Hz, 2H), 1.92 (p, J=6.3 Hz, 2H), 1.86-1.75 (m, 2H), 1.70-1.59 (m, 2H), 1.59-1.50 (m, 2H), 1.47-1.40 (m, 12H).

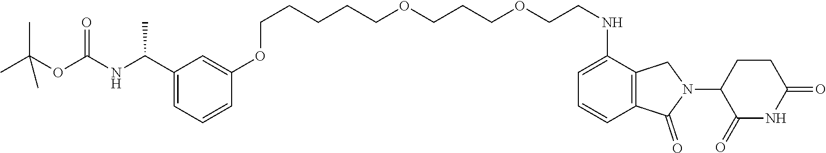 [SMILES: C[C@@H](NC(=O)OC(C)(C)C)c1cccc(OCCCCCOCCCOCCNc2cccc3c2CN(C2CCC(=O)NC2=O)C3=O)c1]

Compound 7. tert-butyl (1R)-1-(3-(5-(3-(2-(2-(2,6-dioxopiperidin-3-yl)-1-oxoisoindolin-4-ylamino)ethoxy) propoxy)pentyloxy)phenyl)ethylcarbamate. To a solution of lenalidomide (0.18 g, 0.69 mmol, 1.0 eq) and compound 6 (0.29 g, 0.68 mmol, 1.0 eq) in 50 mL of DCE was added acetic acid (0.24 mL, 4.18 mmol, 6.0 eq) and sodium triacetoxyborohydride (0.6 g, 2.83 mmol, 4.0 eq). The suspended solution was stirred at room temperature for 12 h., saturated NaHCO₃ aqueous solution added and extracted with dichloromethane (3 by 10 ml). After extraction, the combined organic layer was dried over Na₂SO₄. Purification by silica gel (gradient ethyl acetate/tetrahydrofuran=100:1 to 1:100 as a developing solvent) afforded compound 7 (0.31 g, 69%). LCMS (C18 column 20×2 mm, 2.5 μm, pore size 100 Å, water-acetonitrile+0.1% TFA, gradient 5 to 87% for 3 min, retention time 1.73 min). MS (ESI) m/z 667.3 [MH]+. ¹H NMR (400 MHz, CDCl₃) δ 8.12 (s, 1H), 7.43-7.32 (m, 1H), 7.30 (d, J=3.5 Hz, 1H), 7.24 (t, J=8.0 Hz, 1H), 6.92-6.73 (m, 4H), 5.24 (d, J=12.7 Hz, 1H), 4.90-4.70 (m, 2H), 4.33 (d, J=15.3 Hz, 1H), 4.15 (d, J=14.7 Hz, 1H), 3.96 (dd, J=11.9, 6.0 Hz, 2H), 3.75-3.65 (m, 2H), 3.65-3.34 (m, 8H), 3.28 (q, J=7.0 Hz, 1H), 2.88-2.80 (m, 2H), 2.38-2.30 (m, 1H), 2.25-2.18 (m, 1H), 1.94-1.74 (m, 5H), 1.70-1.61 (m, 3H), 1.59-1.49 (m, 3H), 1.43 (s, 9H).

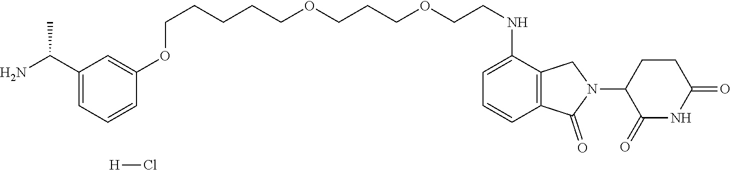

Compound 8. 3-(4-(2-(3-(5-(3-((R)-1-aminoethyl)phenoxy)pentyloxy)propoxy)ethylamino)-1-oxoisoindolin-2-yl)piperidine-2,6-dione hydrochloride. To an ice cold solution of compound 7 (0.31 g, 0.47 mmol) in methylene chloride (10 ml) a dioxanic 3M HCl solution (1 ml) was added. The mixture was stirred at ambient temperature overnight and then evaporated dryness. The residue was taken up with dry ether (20 ml) and the solid was filtered, washed with ether (10 ml) and dried on air. Yield of 8 was 0.28 g (99%) that was used further without purification. LCMS (C18 column 20×2 mm, 2.5 μm, pore size 100 Å, water-acetonitrile+0.1% TFA, gradient 5 to 87% for 3 min, retention time 1.19 min). MS (ESI) m/z 567.8 [MH]+.

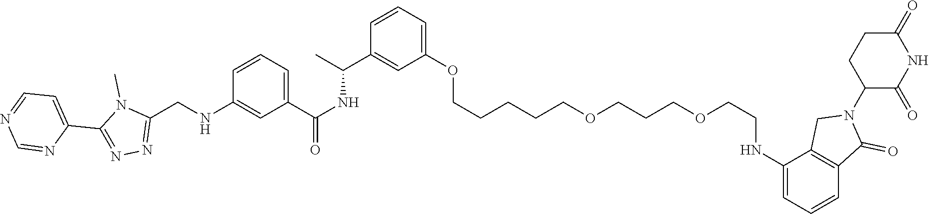 [SMILES: C[C@@H](NC(=O)c1cccc(NCc2nnc(-c3ccncn3)n2C)c1)c1cccc(OCCCCCOCCCOCCNc2cccc3c2CN(C2CCC(=O)NC2=O)C3=O)c1]

Compound 10. D196. N-((1R)-1-(3-(5-(3-(2-(2-(2,6-dioxopiperidin-3-yl)-1-oxoisoindolin-4-ylamino) ethoxy)propoxy)pentyloxy)phenyl)ethyl)-3-((4-methyl-5-(pyrimidin-4-yl)-4H-1,2,4-triazol-3-yl)methylamino)benzamide. To an ice cold suspension of amine hydrochloride 8 (0.1 g, 0.16 mmol) in methylene chloride (5 ml), acid 9 (0.06 g, 0.19 mmol) was added, followed by hydroxyl benzotriazole hydrate (0.032 g, 0.21 mmol) and EDCI (0.04 g, 0.21 mmol) and finally DIPEA (0.5 ml, 3.86 mmol). The reaction mixture was stirred at ambient temperature overnight. Then water (5 ml) was added and organic layer was separated. Aqueous layer was extracted with methylene chloride (5 ml). Combined organic layers were dried over MgSO₄ and evaporated. The residue was purified by HPLC in neutral conditions (YMC-Pack ODS-AQ 250×20 mml, S-10 μm, 12 nm, gradient water-acetonitrile). Yield 0.023 g (16%).

LCMS (C18 column 20×2 mm, 2.5 μm, pore size 100 Å, water-acetonitrile+0.1% TFA, gradient 5 to 87% for 10 min, retention time 5.97 min). MS (ESI) m/z 859.9 [MH]+. ¹H NMR (400 MHz, DMSO) δ 10.99 (s, 1H), 9.33 (d, J=1.2 Hz, 1H), 8.96 (d, J=5.3 Hz, 1H), 8.56 (d, J=8.2 Hz, 1H), 8.17 (dd, J=5.2, 1.4 Hz, 1H), 7.27 (t, J=7.7 Hz, 1H), 7.23-7.14 (m, 3H), 7.10 (d, J=7.6 Hz, 1H), 6.97-6.86 (m, 4H), 6.79 (d, J=8.0 Hz, 1H), 6.75 (d, J=8.0 Hz, 1H), 6.46 (t, J=5.7 Hz, 1H), 5.56 (t, J=5.5 Hz, 1H), 5.10 (dd, J=13.2, 5.2 Hz, 2H), 4.57 (d, J=5.6 Hz, 2H), 4.22 (d, J=17.1 Hz, 1H), 4.12 (d, J=17.1 Hz, 1H), 4.06 (s, 3H), 3.91 (t, J=6.4 Hz, 2H), 3.53 (t, J=5.8 Hz, 2H), 3.45 (t, J=6.4 Hz, 2H), 3.37 (t, J=6.4 Hz, 2H), 3.34-3.28 (m, 2H), 3.00-2.82 (m, 1H), 2.62 (t, J=17.0 Hz, 1H), 2.35-2.22 (m, 1H), 2.06-1.94 (m, 1H), 1.78-1.60 (m, 5H), 1.49 (dd, J=13.7, 6.7 Hz, 2H), 1.45-1.38 (m, 6H).

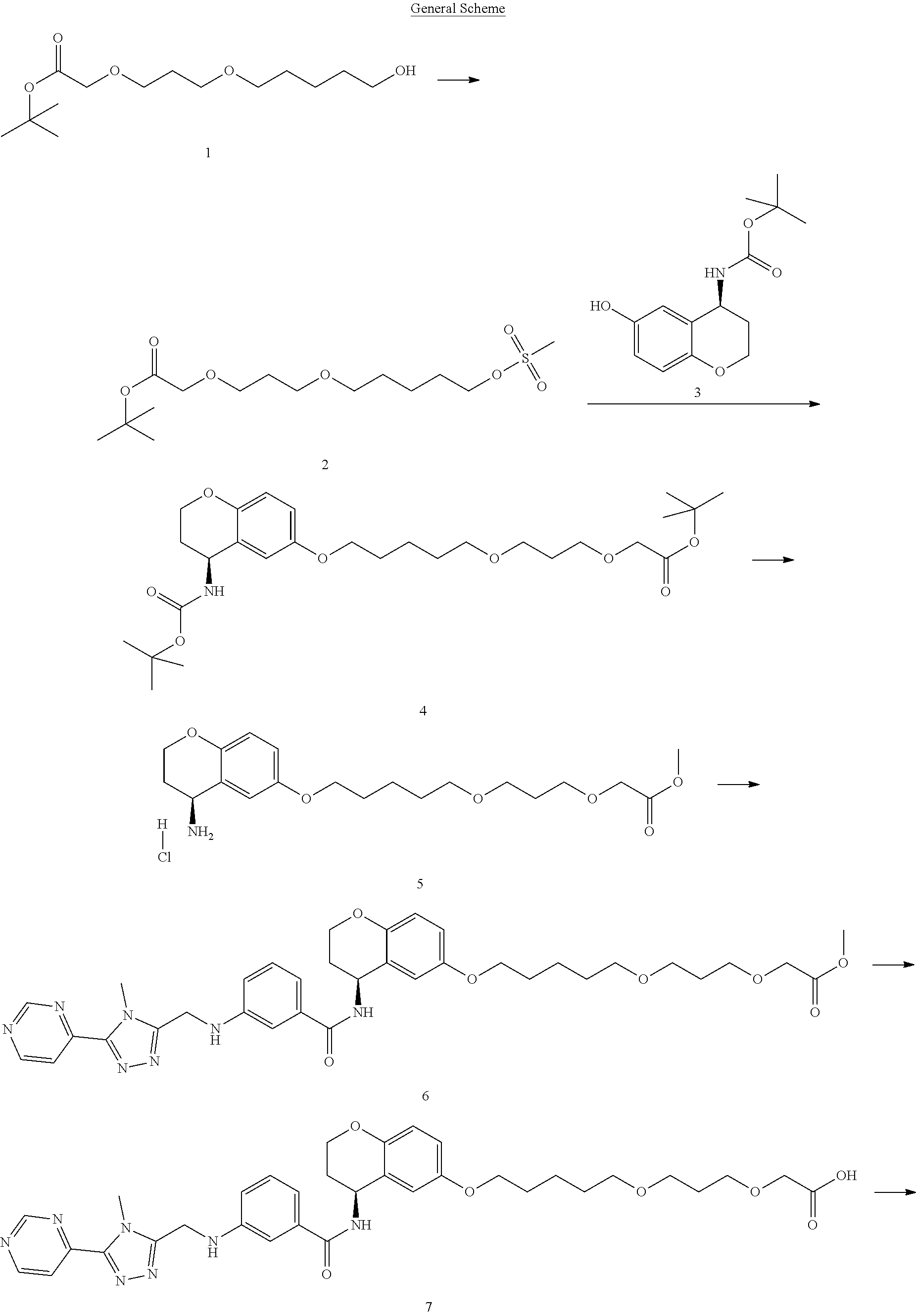

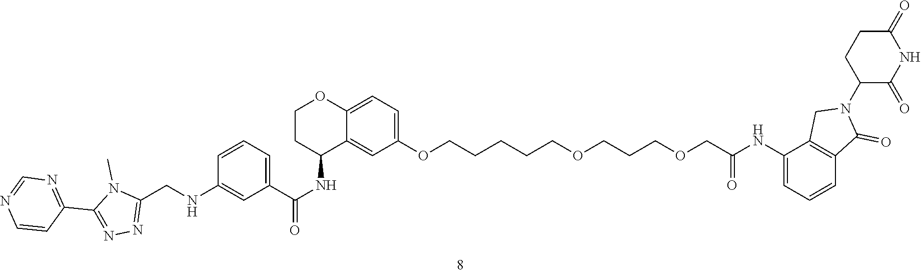 [SMILES: Cn1c(CNc2cccc(C(=O)N[C@H]3CCOc4ccc(OCCCCCOCCCOCC(=O)Nc5cccc6c5CN(C5CCC(=O)NC5=O)C6=O)cc43)c2)nnc1-c1ccncn1]

### Compounds, Procedures, and Analytical Data

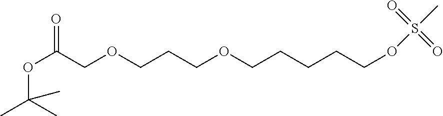 [SMILES: CC(C)(C)OC(=O)COCCCOCCCCCOS(C)(=O)=O]

Compound 2. 5-(3-(2-tert-butoxy-2-oxoethoxy)propoxy)pentyl methanesulfonate. To an ice cold solution of alcohol 1 (0.45 g, 1.63 mmol) in methylene chloride (10 ml), DIPEA (0.4 ml, 0.29 g, 2.31 mmol) was added, followed by mesyl chloride (0.16 ml, 0.23 g, 2.02 mmol). The reaction mixture was stirred at ambient temperature for 4 hours and quenched with water (10 ml). Organic phase was separated; aqueous was extracted with methylene chloride (10 ml). Combined organic phases were washed with water (10 ml) and dried over MgSO₄. The solvent was stripped off and the product 2 was used further without additional purification. Yield 0.58 g (100%).

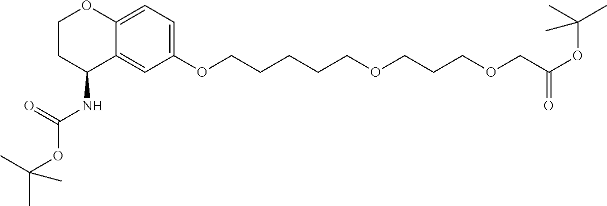 [SMILES: CC(C)(C)OC(=O)COCCCOCCCCCOc1ccc2c(c1)[C@@H](NC(=O)OC(C)(C)C)CCO2]

Compound 4. (S)-tert-butyl 2-(3-(5-(4-(tert-butoxycarbonylamino)chroman-6-yloxy)pentyloxy) propoxy)acetate. Compound 3 (0.4 g, 1.50 mmol), compound 2 (0.58 g, 1.63 mmol) and K₂CO₃ (0.52 g, 3.76 mmol, in dry acetonitrile (50 mL) were stirred for 12 h at reflux. The solvent was evaporated to dryness. The residue was partitioned between methylene chloride (10 ml) and water (10 ml). Organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 0.8 g (89%). LCMS (C18 column 20×2 mm, 2.5 μm, pore size 100 Å, water-acetonitrile+0.1% TFA, gradient 5 to 87% for 3 min, retention time 2.08 min). MS (ESI) m/z 524.8 [MH]+.

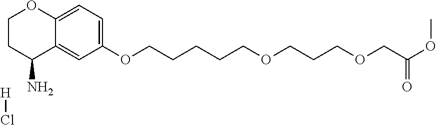

Compound 5. (S)-methyl 2-(3-(5-(4-aminochroman-6-yloxy)pentyloxy)propoxy)acetate hydrochloride To an ice cold solution of compound 4 (0.8 g, 1.5 mmol) in methylene chloride (10 ml), dioxanic HCl solution (3.0 ml, 3M) was added and the mixture was stirred at room temperature for overnight. Then the solvent was evaporated, methanol (50 ml) was added and the solution was cooled in ice bath. SOCl2 (4 ml, 6.68 g, 56.13 mmol) was added drop-wise. The reaction was kept at ambient temperature and evaporated dryness. The residue was crude 5 and used further. Yield 0.7 g (100%). LCMS (C18 column 20×2 mm, 2.5 μm, pore size 100 Å, water-acetonitrile+0.1% TFA, gradient 5 to 87% for 3 min, retention time 1.16 min). MS (ESI) m/z 382.5 [MH]+.

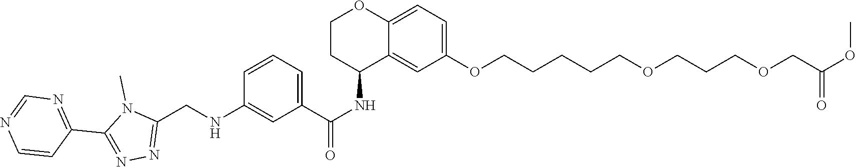 [SMILES: COC(=O)COCCCOCCCCCOc1ccc2c(c1)[C@@H](NC(=O)c1cccc(NCc3nnc(-c4ccncn4)n3C)c1)CCO2]

Compound 6. (S)-methyl 2-(3-(5-(4-(3-((4-methyl-5-(pyrimidin-4-yl)-4H-1,2,4-triazol-3-yl)methylamino)benzamido)chroman-6-yloxy)pentyloxy)propoxy)acetate. To an ice cold suspension of amine hydrochloride 5 (0.20 g, 0.48 mmol) in DCM (10 ml), 3-((4-methyl-5-(pyrimidin-4-yl)-4H-1,2,4-triazol-3-yl)methylamino)benzoic acid (0.15 g, 0.48 mmol) was added, followed by hydroxyl benzotriazole hydrate (0.10 g, 0.65 mmol) and EDCI (0.13 g, 0.65 mmol) and finally DIPEA (0.5 ml, 0.37 g, 2.89 mmol). The reaction mixture was stirred at ambient temperature overnight. Then the reaction was diluted with water (10 ml) and organic layer was separated. Aqueous layer was extracted with methylene chloride (10 ml). Combined organic layers were dried over MgSO4 and evaporated. The residue was purified by chromatography on silica, using chloroform-methanol (20 to 1) mixture as an eluent. Yield of product 6 was 0.22 g (70%). LCMS (C18 column 20×2 mm, 2.5 μm, pore size 100 Å, water-acetonitrile+0.1% TFA, gradient 5 to 87% for 3 min, retention time 1.45 min). MS (ESI) m/z 674.5 [MH]+.

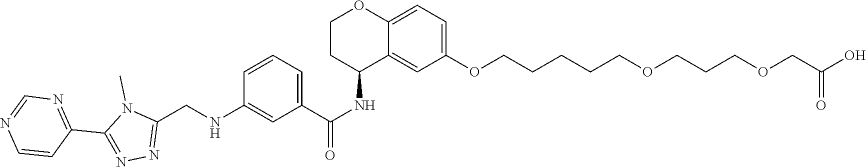 [SMILES: Cn1c(CNc2cccc(C(=O)N[C@H]3CCOc4ccc(OCCCCCOCCCOCC(=O)O)cc43)c2)nnc1-c1ccncn1]

Compound 7. (S)-2-(3-(5-(4-(3-((4-methyl-5-(pyrimidin-4-yl)-4H-1,2,4-triazol-3-yl)methylamino) benzamido)chroman-6-yloxy)pentyloxy)propoxy)acetic acid. To a solution of ester 6 (0.22 g, 0.33 mmol) in methanol (5 ml), solid KOH (0.1 g, 1.78 mmol) was added and the mixture was stirred for 48 hours. The solvent was stripped off and the residue was treated with a solution of KHSO4 (0.24 g, 1.78 mmol) in water (5 ml). The reaction mixture was stirred for an hour and the organic layer was separated; aqueous one was extracted with methylene chloride (5 ml). Combined organic layers were dried over MgSO4 and concentrated. Yield 0.21 g, (97%). LCMS (C18 column 20×2 mm, 2.5 μm, pore size 100 Å, water-acetonitrile+0.1% TFA, gradient 5 to 87% for 3 min, retention time 1.34 min). MS (ESI) m/z 660.5 [MH]+.

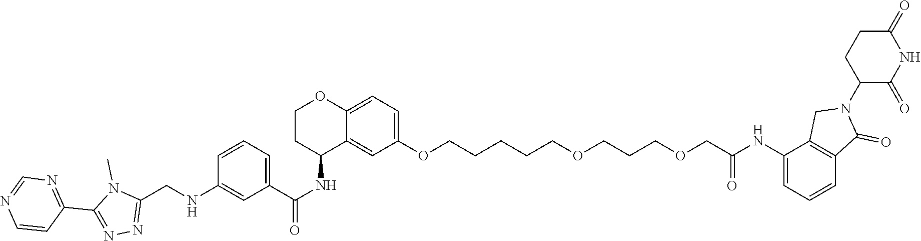 [SMILES: Cn1c(CNc2cccc(C(=O)N[C@H]3CCOc4ccc(OCCCCCOCCCOCC(=O)Nc5cccc6c5CN(C5CCC(=O)NC5=O)C6=O)cc43)c2)nnc1-c1ccncn1]

Compound 8. D155. N-((4S)-6-(5-(3-(2-(2-(2,6-dioxopiperidin-3-yl)-1-oxoisoindolin-4-ylamino)-2-oxoethoxy)propoxy)pentyloxy)chroman-4-yl)-3-((4-methyl-5-(pyrimidin-4-yl)-4H-1,2,4-triazol-3-yl)methylamino)benzamide. To a solution of acid 7 (0.2 g, 0.30 mmol) in dried pyridine (2 ml), 3-(4-amino-1-oxoisoindolin-2-yl)piperidine-2,6-dione (0.08 g, 0.31 mmol) was added, followed by TBTU (1.2 g, 0.31 mmol) and DIPEA (0.2 ml, 1.15 mmol). The reaction mixture was stirred at ambient temperature and evaporated dryness. The residue was partitioned between methylene chloride (5 ml) and water (5 ml). The organic layer was separated; aqueous one was extracted with methylene chloride (5 ml). Combined organic layers were dried over MgSO4 and concentrated. The product 8 was purified by HPLC chromatography. Yield 0.178 g (65%). LCMS (C18 column 20×2 mm, 2.5 μm, pore size 100 Å, water-acetonitrile+0.1% TFA, gradient 5 to 87% for 10 min, retention time 5.63 min). MS (ESI) m/z 901.6 [MH]+.

¹H NMR (400 MHz, DMSO-d₆) δ 10.99 (s, 1H), 9.68 (s, 1H), 9.33 (d, J=1.3 Hz, 1H), 8.96 (d, J=5.3 Hz, 1H), 8.64 (d, J=8.3 Hz, 1H), 8.17 (dd, J=5.3, 1.4 Hz, 1H), 7.73 (d, J=7.6 Hz, 1H), 7.55 (d, J=6.6 Hz, 1H), 7.50 (t, J=7.6 Hz, 1H), 7.26 (s, 1H), 7.20-7.08 (m, 2H), 6.90 (d, J=8.7 Hz, 1H), 6.75-6.66 (m, 3H), 6.45 (t, J=5.7 Hz, 1H), 5.22 (dd, J=14.1, 7.1 Hz, 1H), 5.13 (dd, J=13.2, 5.1 Hz, 1H), 4.57 (d, J=5.6 Hz, 2H), 4.37 (q, J=17.5 Hz, 2H), 4.29-4.20 (m, 1H), 4.20-4.10 (m, 1H), 4.10-4.02 (m, 6H), 3.80 (t, J=6.4 Hz, 2H), 3.56 (t, J=6.4 Hz, 2H), 3.43 (t, J=6.4 Hz, 2H), 2.97-2.83 (m, 1H), 2.68-2.55 (m, 2H), 2.42-2.27 (m, 1H), 2.15-1.93 (m, 4H), 1.79 (p, J=6.3 Hz, 2H), 1.60 (dd, J=14.3, 6.9 Hz, 2H), 1.46 (dd, J=14.3, 6.5 Hz, 2H), 1.41-1.28 (m, 2H).

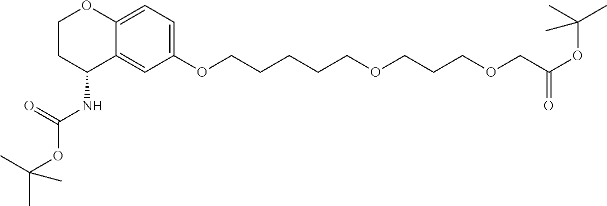 [SMILES: CC(C)(C)OC(=O)COCCCOCCCCCOc1ccc2c(c1)[C@H](NC(=O)OC(C)(C)C)CCO2]

Compound 4. (R)-tert-butyl 2-(3-(5-(4-(tert-butoxycarbonylamino)chroman-6-yloxy)pentyloxy)propoxy)acetate. Compound 3 (enantiomer) (0.4 g, 1.50 mmol), compound 2 (0.25 g, 0.94 mmol) and K₂CO₃ (0.32 g, 2.3 mmol, in dry acetonitrile (50 mL) were stirred for 12 h at reflux. The solvent was evaporated to dryness. The residue was partitioned between methylene chloride (10 ml) and water (10 ml). Organic layer was separated; aqueous one was extracted with methylene chloride (10 ml). Combined organic layers were dried over MgSO₄ and concentrated. The residue was purified by silica gel flash column chromatography using a mixture of chloroform and methanol (100 to 1 v/v) as an eluent to afford the desired product 4. Yield 0.2 g (40%). LCMS (C18 column 20×2 mm, 2.5 μm, pore size 100 Å, water-acetonitrile+0.1% TFA, gradient 5 to 87% for 3 min, retention time 2.10 min). MS (ESI) m/z 524.5 [MH]+.

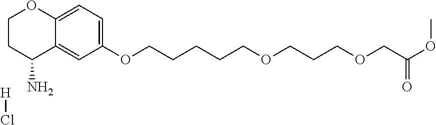

Compound 5. (R)-methyl 2-(3-(5-(4-aminochroman-6-yloxy)pentyloxy)propoxy)acetate hydrochloride To an ice cold solution of compound 4 (0.2 g, 1.5 mmol) in methylene chloride (10 ml), dioxanic HCl solution (0.7 ml, 3M) was added and the mixture was stirred at room temperature for overnight. Then the solvent was evaporated, methanol (50 ml) was added and the solution was cooled in ice bath. SOCl2 (0.3 ml, 0.5 g, 4.21 mmol) was added drop-wise. The reaction was kept at ambient temperature and evaporated dryness. The residue was crude 5 and used further. Yield 0.18 g (100%). LCMS (C18 column 20×2 mm, 2.5 μm, pore size 100 Å, water-acetonitrile+0.1% TFA, gradient 5 to 87% for 3 min, retention time 1.10 min). MS (ESI) m/z 382.5 [MH]+.

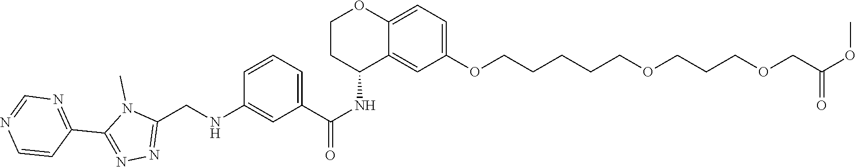 [SMILES: COC(=O)COCCCOCCCCCOc1ccc2c(c1)[C@H](NC(=O)c1cccc(NCc3nnc(-c4ccncn4)n3C)c1)CCO2]

Compound 6. (R)-methyl 2-(3-(5-(4-(3-((4-methyl-5-(pyrimidin-4-yl)-4H-1,2,4-triazol-3-yl)methylamino)benzamido)chroman-6-yloxy)pentyloxy)propoxy)acetate. To an ice cold suspension of amine hydrochloride 5 (0.06 g, 0.14 mmol) in DCM (10 ml), 3-((4-methyl-5-(pyrimidin-4-yl)-4H-1,2,4-triazol-3-yl)methylamino)benzoic acid (0.04 g, 0.14 mmol) was added, followed by hydroxyl benzotriazole hydrate (0.02 g, 0.17 mmol) and EDCI (0.04 g, 0.22 mmol) and finally DIPEA (0.2 ml, 0.15 g, 1.15 mmol). The reaction mixture was stirred at ambient temperature overnight. Then the reaction was diluted with water (10 ml) and organic layer was separated. Aqueous layer was extracted with methylene chloride (10 ml). Combined organic layers were dried over MgSO4 and evaporated. The residue was purified by chromatography on silica, using chloroform-methanol (20 to 1) mixture as an eluent. Yield of product 6 was 0.11 g (98%).

LCMS (C18 column 20×2 mm, 2.5 μm, pore size 100 Å, water-acetonitrile+0.1% TFA, gradient 5 to 87% for 3 min, retention time 1.42 min). MS (ESI) m/z 674.5 [MH]+.

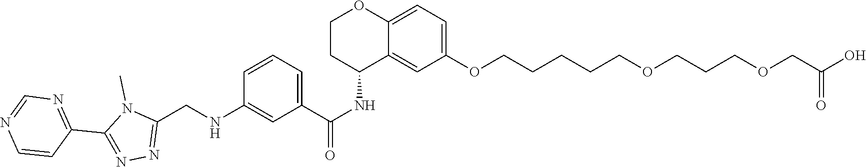 [SMILES: Cn1c(CNc2cccc(C(=O)N[C@@H]3CCOc4ccc(OCCCCCOCCCOCC(=O)O)cc43)c2)nnc1-c1ccncn1]

Compound 7. (R)-2-(3-(5-(4-(3-((4-methyl-5-(pyrimidin-4-yl)-4H-1,2,4-triazol-3-yl)methylamino) benzamido)chroman-6-yloxy)pentyloxy)propoxy)acetic acid. To a solution of ester 6 (0.11 g, 0.16 mmol) in methanol (5 ml), solid NaOH (0.056 g, 1.4 mmol) was added and the mixture was stirred for 48 hours. The solvent was stripped off and the residue was treated with a solution of KHSO4 (0.19 g, 1.4 mmol) in water (5 ml). The reaction mixture was stirred for an hour and the organic layer was separated; aqueous one was extracted with methylene chloride (5 ml). Combined organic layers were dried over MgSO4 and concentrated. Yield 0.08 g, (77%). LCMS (C18 column 20×2 mm, 2.5 μm, pore size 100 Å, water-acetonitrile+0.1% TFA, gradient 5 to 87% for 3 min, retention time 1.33 min). MS (ESI) m/z 660.5 [MH]+.

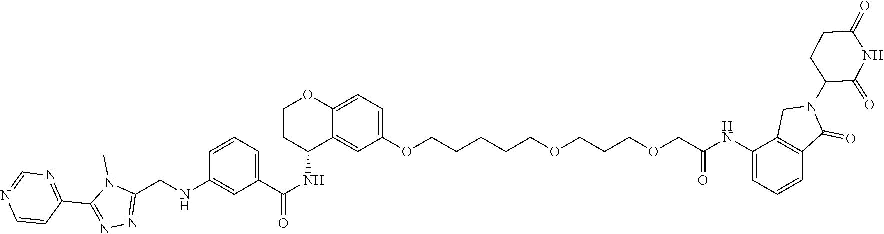 [SMILES: Cn1c(CNc2cccc(C(=O)N[C@@H]3CCOc4ccc(OCCCCCOCCCOCC(=O)Nc5cccc6c5CN(C5CCC(=O)NC5=O)C6=O)cc43)c2)nnc1-c1ccncn1]

Compound 8. D193. N-((4R)-6-(5-(3-(2-(2-(2,6-dioxopiperidin-3-yl)-1-oxoisoindolin-4-ylamino)-2-oxoethoxy)propoxy)pentyloxy)chroman-4-yl)-3-((4-methyl-5-(pyrimidin-4-yl)-4H-1,2,4-triazol-3-yl)methylamino)benzamide. To a solution of acid 7 (0.08 g, 0.12 mmol) in dried pyridine (2 ml), 3-(4-amino-1-oxoisoindolin-2-yl)piperidine-2,6-dione (0.05 g, 0.19 mmol) was added, followed by TBTU (0.08 g, 2.11 mmol) and DIPEA (0.2 ml, 1.15 mmol). The reaction mixture was stirred at ambient temperature and evaporated dryness. The residue was partitioned between methylene chloride (5 ml) and water (5 ml). The organic layer was separated; aqueous one was extracted with methylene chloride (5 ml). Combined organic layers were dried over MgSO4 and concentrated. The product 8 was purified by HPLC chromatography. Yield 0.052 g (35%). LCMS (C18 column 20×2 mm, 2.5 μm, pore size 100 Å, water-acetonitrile+0.1% TFA, gradient 5 to 87% for 10 min, retention time 5.53 min). MS (ESI) m/z 901.6 [MH]+. ¹H NMR (400 MHz, DMSO-d₆) δ 11.00 (s, 1H), 9.68 (s, 1H), 9.33 (d, J=1.3 Hz, 1H), 8.97 (d, J=5.3 Hz, 1H), 8.65 (d, J=8.3 Hz, 1H), 8.17 (dd, J=5.2, 1.4 Hz, 1H), 7.73 (d, J=7.7 Hz, 1H), 7.55 (d, J=6.5 Hz, 1H), 7.50 (t, J=7.6 Hz, 1H), 7.26 (s, 1H), 7.16 (t, J=7.7 Hz, 1H), 7.12 (d, J=7.7 Hz, 1H), 6.90 (d, J=8.7 Hz, 1H), 6.75-6.66 (m, 3H), 6.46 (t, J=5.5 Hz, 1H), 5.22 (dd, J=13.6, 6.9 Hz, 1H), 5.13 (dd, J=13.3, 5.1 Hz, 1H), 4.57 (d, J=5.5 Hz, 2H), 4.37 (q, J=17.4 Hz, 2H), 4.26-4.21 (m, 1H), 4.17-4.12 (m, 1H), 4.07 (s, 5H), 3.80 (t, J=6.4 Hz, 2H), 3.56 (t, J=6.4 Hz, 2H), 3.43 (t, J=6.4 Hz, 2H), 3.32 (t, J=6.4 Hz, 2H), 2.97-2.83 (m, 1H), 2.69-2.55 (m, 1H), 2.35 (dd, J=13.1, 4.2 Hz, 1H), 2.10-1.96 (m, 3H), 1.84-1.72 (m, 2H), 1.60 (dd, J=14.2, 6.8 Hz, 2H), 1.45 (dd, J=13.9, 6.7 Hz, 2H), 1.40-1.28 (m, 2H).

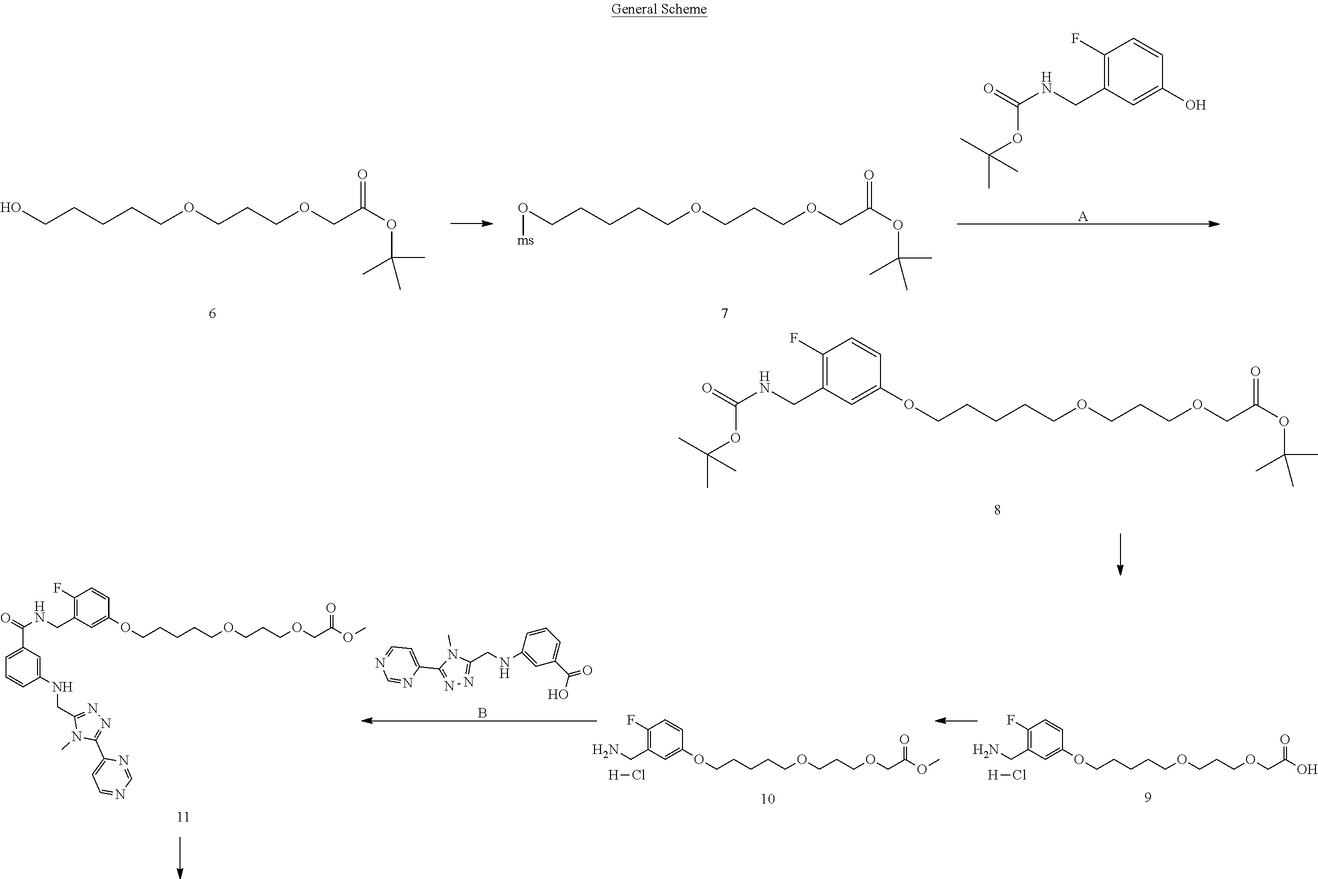

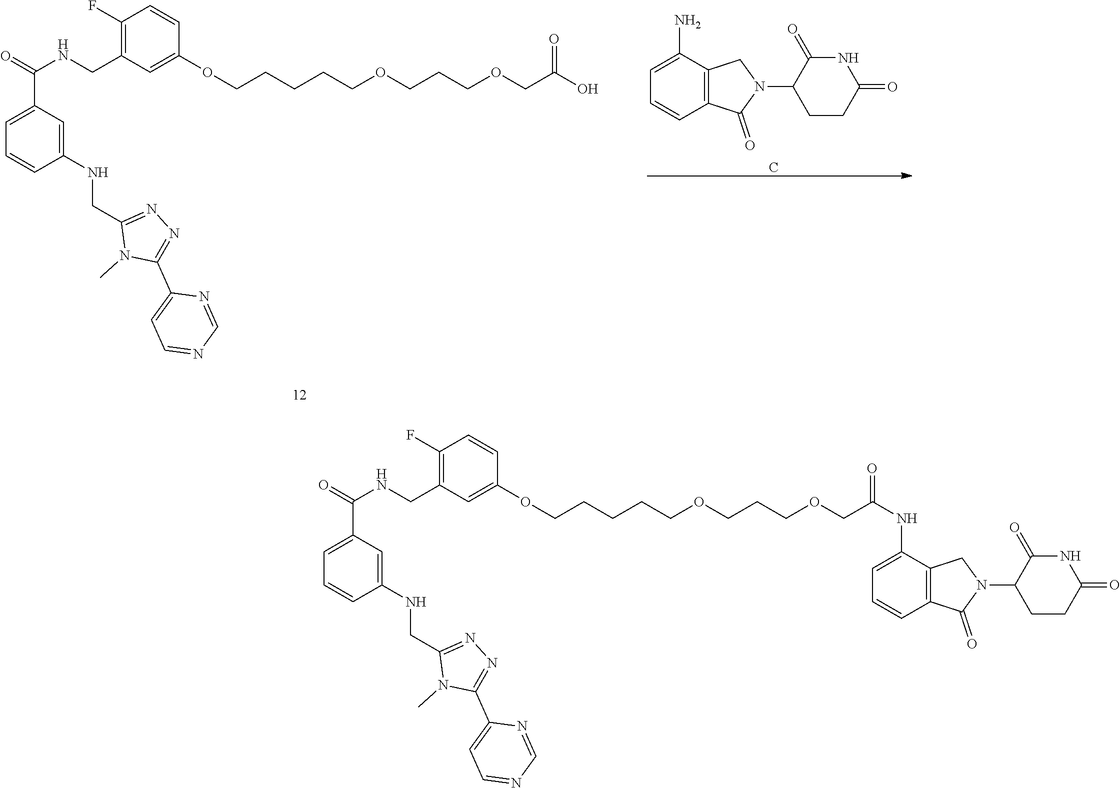

### General Procedure

Compound 7. Solution of compound 6 (1.8 mmol, 1 eq) in CH₂Cl₂ was mixed with DIPEA (2.7 mmol, 1.5 eq) and cooled to 0° C. 2.2 mmol (1.2 eq) of MsCl was added dropwise. The mixture was stirred at room temperature for 2 hours, washed with water, dried over Na₂SO₄ and evaporated. The product was used without purification.

Compound 8. A mixture of compound A (1.6 mmol, 1 eq), compound 7 (1.8 mmol, 1.1 eq), K₂CO₃ (3.2 mmol, 2 eq) and anh. acetonitrile (50 mL) was stirred and heated under reflux for 12 h, filtered, and concentrated under reduced pressure. The residue was purified by silica gel column chromatography using chloroform and methanol (80:1) as an eluent to afford the desired product 8.

Compound 9. A solution of compound 8 (1.7 mmol, 1 eq) in dry DCM (50 mL) with 2.8 ml 3 M HCl in dioxane (5 eq) were stirred for 24 h at r.t. The mixture was evaporated to dryness to give the crude product 9 which was used in the next step without additional purification.

Compound 10. SOCl₂ (0.26 ml, 3.6 mmol, 2 eq) was added dropwise to a stirred solution of compound 9 (1.8 mmol, 1 eq) in dry MeOH (20 mL). The mixture was stirred for 12 h at r.t. and then evaporated to dryness to give the crude product 11 that was used for the next step without additional purification.

Compound 11. A mixture of compound 10 (0.5 mmol, 1 eq), compound B (0.5 mmol, 1 eq), HOBt (0.61 mmol, 1.2 eq), EDCI (0.6 mmol, 1.1 eq), DIPEA (2.0 mmol, 4 eq), and anh DCM (30 mL) was stirred for 12 h at ambient temperature, washed with water (3*10 ml), brine (2×10 mL), dried over Na₂SO₄, and concentrated under reduced pressure at ambient temperature. The residue was purified by silica gel flash column chromatography using chloroform and methanol as an eluent to afford the desired product 11.

Compound 12. To a solution of compound 11 (0.44 mmol, 1 eq) in MeOH (20 mL) was added a solution KOH (1.32 mmol, 3 eq) in 3 mL H₂O. The mixture was stirred for 12-36 h at ambient temperature and concentrated under reduced pressure. The residue was treated with H₂O (15 mL), and the mixture was acidified to pH 4 with 1N aq. HCl and extracted with of DCM (4*15 mL). Combined organic layers were washed with brine, dried over Na₂SO₄ and evaporated. The product was used for the next step without additional purification.

D197. A mixture of compound 12 (0.1 mmol, 1 eq), compound C (0.1 mmol, 1 eq), TBTU (0.2 mmol, 2 eq), DIPEA (0.4 mmol, 4 eq), and anh. pyridine (3 mL) was stirred at ambient temperature for 12 h and concentrated under reduced pressure. The residue was treated with 3M solution of HCl in dioxane, and the mixture was evaporated to dryness. The residue was purified by HPLC in acidic conditions (YMC-Pack ODS-AQ 250×20 mml, S-10 μm, 12 nm, gradient water-acetonitrile).

### Syntheses According to the General Procedures

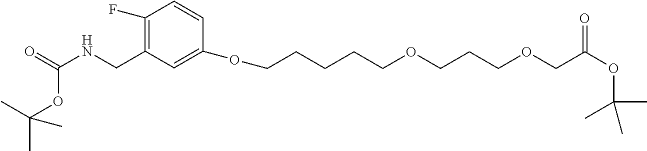 [SMILES: CC(C)(C)OC(=O)COCCCOCCCCCOc1ccc(F)c(CNC(=O)OC(C)(C)C)c1]

Compound 8. tert-butyl 2-(3-(5-(3-((tert-butoxycarbonylamino)methyl)-4-fluorophenoxy)pentyloxy) propoxy)acetate. Yield 75%. LCMS (C18 column 20×2 mm, 2.5 μm, pore size 100 Å, water-acetonitrile+0.1% TFA, gradient 5 to 87% for 3 min, retention time 2.03 min). MS (ESI) m/z 500.6 [MH]+. ¹H NMR (400 MHz, CDCl₃) δ 7.28 (s, 1H), 6.94 (m, 1H), 6.85 (m, 1H), 6.74 (m, 1H), 4.91 (br, 1H), 4.34 (br, 2H), 3.96-3.91 (m, 4H), 3.63-3.59 (m, 2H), 3.55-3.52 (m, 2H), 3.47-3.43 (m, 2H), 1.92-1.89 (m, 2H), 1.80-1.78 (m, 2H), 1.66-1.60 (m, 2H), 1.48 (s, 9H), 1.46 (s, 9H), 1.37-1.34 (m, 2H)

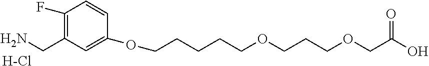

Compound 9. 2-(3-(5-(3-(aminomethyl)-4-fluorophenoxy)pentyloxy)propoxy)acetic acid hydrochloride. Yield 99%. LCMS (C18 column 20×2 mm, 2.5 μm, pore size 100 Å, water-acetonitrile+0.1% TFA, gradient 5 to 87% for 3 min, retention time 1.03 min). MS (ESI) m/z 344.5 [MH]+.

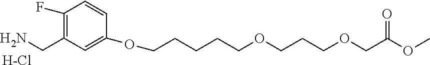

Compound 10. methyl 2-(3-(5-(3-(aminomethyl)-4-fluorophenoxy)pentyloxy)propoxy)acetate hydrochloride. Light brown solid. Quantitative yield. LCMS (C18 column 20×2 mm, 2.5 μm, pore size 100 Å, water-acetonitrile+0.1% TFA, gradient 5 to 87% for 3 min, retention time 1.11 min). MS (ESI) m/z 358.5 [MH]+.

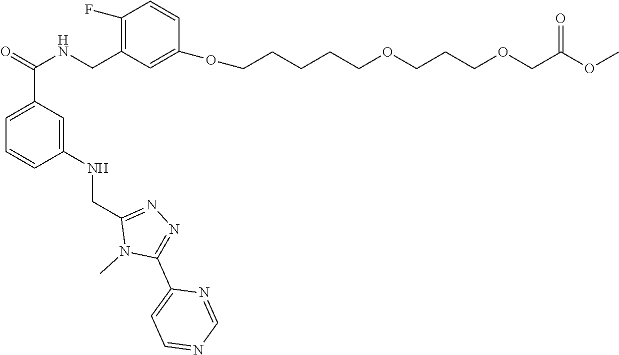 [SMILES: COC(=O)COCCCOCCCCCOc1ccc(F)c(CNC(=O)c2cccc(NCc3nnc(-c4ccncn4)n3C)c2)c1]

Compound 11. methyl 2-(3-(5-(4-fluoro-3-((3-((4-methyl-5-(pyrimidin-4-yl)-4H-1,2,4-triazol-3-yl)methylamino)benzamido)methyl)phenoxy)pentyloxy)propoxy)acetate. Yield 86%. LCMS (C18 column 20×2 mm, 2.5 μm, pore size 100 Å, water-acetonitrile+0.1% TFA, gradient 5 to 87% for 3 min, retention time 1.44 min). MS (ESI) m/z 650.3 [MH]+.

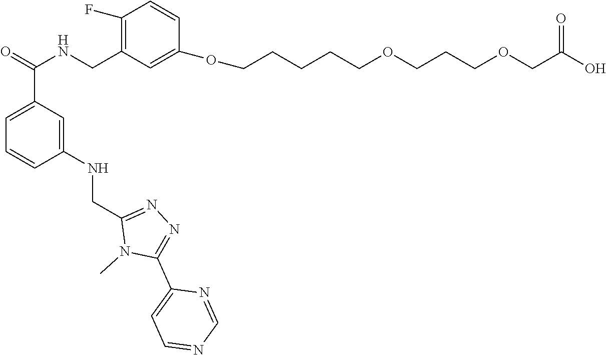 [SMILES: Cn1c(CNc2cccc(C(=O)NCc3cc(OCCCCCOCCCOCC(=O)O)ccc3F)c2)nnc1-c1ccncn1]

Compound 12. 2-(3-(5-(4-fluoro-3-((3-((4-methyl-5-(pyrimidin-4-yl)-4H-1,2,4-triazol-3-yl)methylamino)benzamido)methyl)phenoxy)pentyloxy)propoxy)acetic acid. Yield 80%. LCMS (C18 column 20×2 mm, 2.5 μm, pore size 100 Å, water-acetonitrile+0.1% TFA, gradient 5 to 87% for 3 min, retention time 1.32 min). MS (ESI) m/z 636.5 [MH]+.

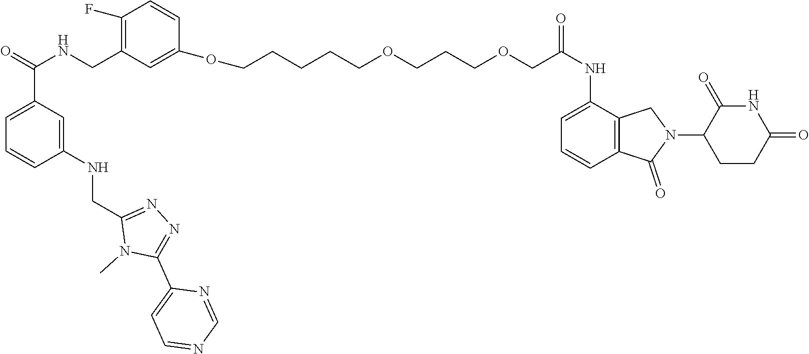 [SMILES: Cn1c(CNc2cccc(C(=O)NCc3cc(OCCCCCOCCCOCC(=O)Nc4cccc5c4CN(C4CCC(=O)NC4=O)C5=O)ccc3F)c2)nnc1-c1ccncn1]

D197. N-(5-(5-(3-(2-(2-(2,6-dioxopiperidin-3-yl)-1-oxoisoindolin-4-ylamino)-2-oxoethoxy)propoxy)pentyloxy)-2-fluorobenzyl)-3-((4-methyl-5-(pyrimidin-4-yl)-4H-1,2,4-triazol-3-yl)methylamino)benzamide. Yield 74%. LCMS (C18 column 100×4.6 mm, 5.0 μm, pore size 100 Å, water-acetonitrile+0.1% TFA, gradient 5 to 87% for 10 min, retention time 5.59 min). MS (ESI) m/z 877.6 [MH]+. ¹H NMR (400 MHz, DMSO-d₆) δ 11.01 (s, 1H), 9.68 (s, 1H), 9.33 (d, J=1.3 Hz, 1H), 8.97 (d, J=5.3 Hz, 1H), 8.80 (t, J=5.8 Hz, 1H), 8.17 (dd, J=5.3, 1.3 Hz, 1H), 7.72 (d, J=8 Hz, 1H), 7.56-7.48 (m, 2H), 7.24 (br, 1H), 7.20-7.05 (m, 3H), 6.91 (dd, J=7.8, 1.5 Hz, 1H), 6.84-6.79 (m, 2H), 6.48 (t, J=5.3, 1H), 5.12 (dd, J=13.2, 5.0 Hz, 1H), 4.56 (d, J=5.6 Hz, 2H), 4.44 (d, J=6.2 Hz, 2H), 4.38 (d, J=8.5 Hz, 2H), 4.07 (s, 5H), 3.86 (t, J=6.4 Hz, 2H), 3.56 (t, J=6.4 Hz, 2H), 3.44 (t, J=6.4 Hz, 2H), 3.33-3.31 (m, 2H), 2.95-2.86 (m, 1H), 2.66-2.57 (m, 1H), 2.41-2.30 (m, 1H), 2.03-2.00 (m, 1H), 1.82-1.76 (m, 2H), 1.67-1.61 (m, 2H), 1.51-1.45 (m, 2H), 1.41-1.33 (m, 2H).

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (A-I): 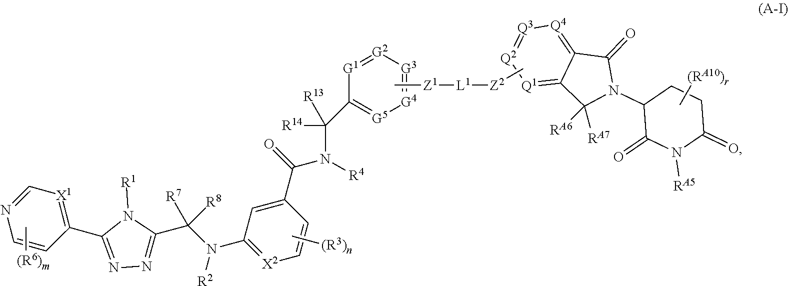
or a pharmaceutically acceptable salt, stereoisomer, tautomer, or isotopically labeled derivative thereof,
wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, or optionally substituted 5-10 membered heteroarylene;
Z¹ is a bond, —O—, —NR^{N}—, —S—, —C(═O)—, or optionally substituted —CH₂—;
Z² is a bond, —O—, —NR^{A11}—, —S—, —C(═O)—, or optionally substituted —CH₂—;
G¹ is CR¹⁵ or N;
G², G³, G⁴, and G⁵ are each independently CR¹⁶, CH, or N;
Q¹, Q², Q³, and Q⁴ are each independently CR^{A1}, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{A5}, and R^{A11} are each independently hydrogen, optionally substituted C₁₋₆ alkyl, optionally substituted C₁₋₆ acyl, or a nitrogen protecting group;
each instance of R³, R⁶, R⁹, R¹⁵, R¹⁶, and R^{A1} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl,
optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R⁸ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{A6} and R^{A7} are independently hydrogen or optionally substituted C₁₋₆ alkyl, or optionally R^{A6} and R^{A7} are joined together with the intervening atoms to form optionally substituted C₃₋₈ carbocyclyl or 3-8 membered heterocyclyl, or optionally R^{A6} and R^{A7} are taken together to form ═O;
each instance of R^{A10} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
m and n are each independently is 0, 1, 2, or 3; and
r is 0, 1, 2, 3, 4, or 5.

2. The compound of claim 1, wherein the compound is of Formula (A-II): 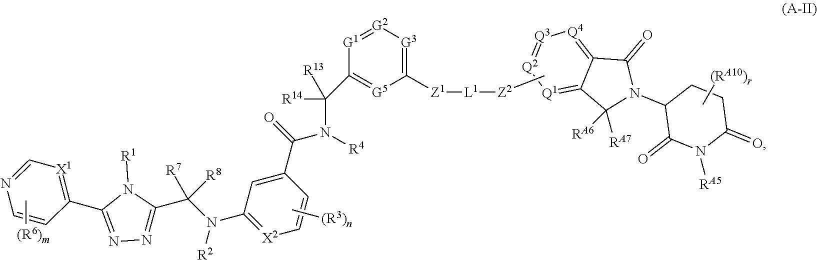
or a pharmaceutically acceptable salt, stereoisomer, tautomer, or isotopically labeled derivative thereof.

3. The compound of claim 1 or 2, wherein the compound is of Formula (A-III): 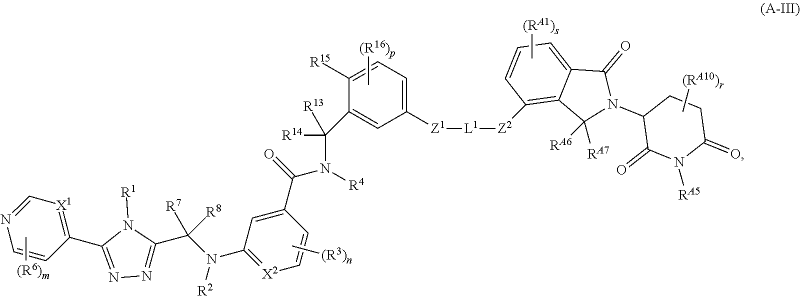
or a pharmaceutically acceptable salt, stereoisomer, tautomer, or isotopically labeled derivative thereof,
wherein p and s are each independently 0, 1, 2, or 3.

4. The compound of any one of claims 1-3, wherein the compound is of Formula (A-IV): 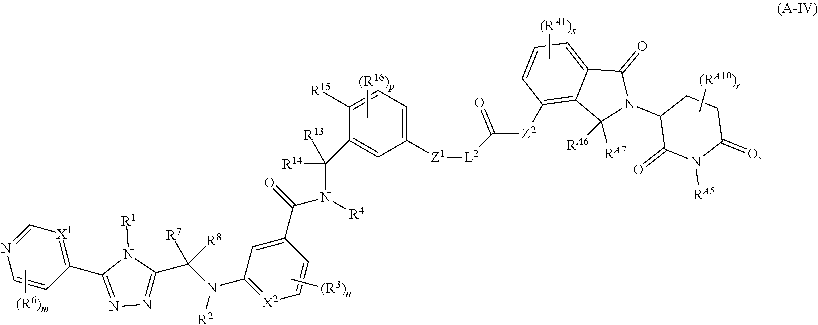
or a pharmaceutically acceptable salt, stereoisomer, tautomer, or isotopically labeled derivative thereof,
wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

5. The compound of claim 1, wherein the compound is of Formula (A-V): 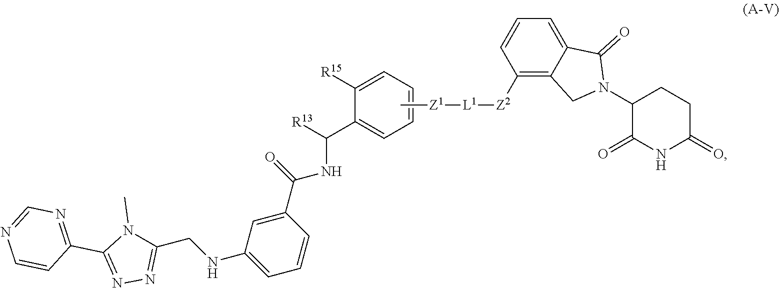
or a pharmaceutically acceptable salt, stereoisomer, tautomer, or isotopically labeled derivative thereof.

6. The compound of claim 3, wherein the compound is of Formula (A-VI): 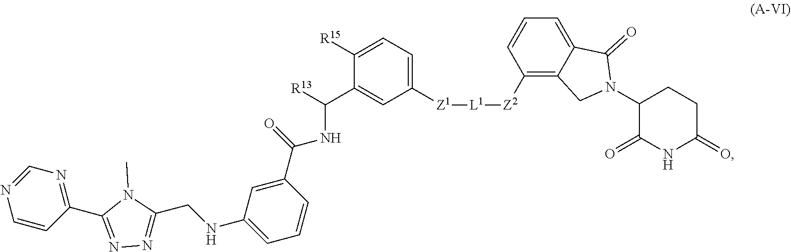
or a pharmaceutically acceptable salt, stereoisomer, tautomer, or isotopically labeled derivative thereof.

7. The compound of claim 4, wherein the compound is of Formula (A-VII): 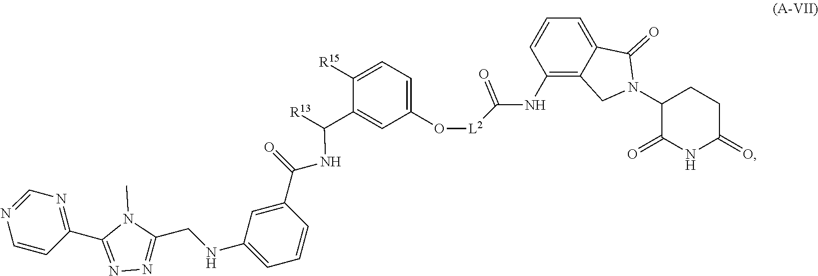
or a pharmaceutically acceptable salt, stereoisomer, tautomer, or isotopically labeled derivative thereof.

8. A compound of Formula (B-I): 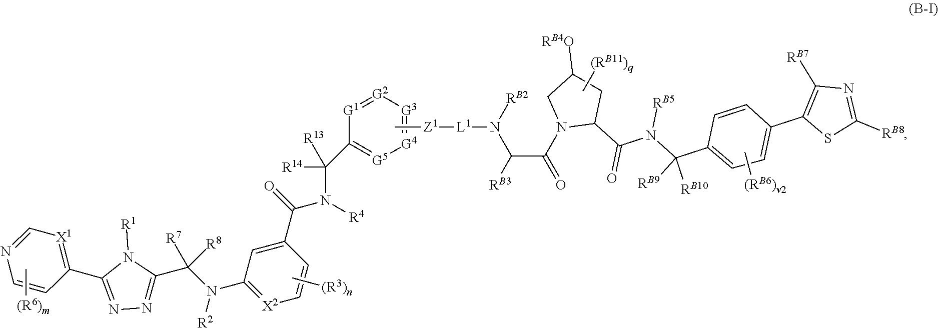
or a pharmaceutically acceptable salt, stereoisomer, tautomer, or isotopically labeled derivative thereof,
wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof;
Z¹ is a bond, —O—, —NR^{N}—, —S—, —C(═O)—, or optionally substituted —CH₂—;
G¹ is CR¹⁵ or N;
G², G³, G⁴, and G⁵ are each independently CR¹⁶, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{B2}, and R^{B5} are each independently hydrogen, optionally substituted C₁₋₆ alkyl, optionally substituted C₁₋₆ acyl, or a nitrogen protecting group;
each instance of R³, R⁶, R⁹, R¹⁵, R¹⁶, R^{B6}, R^{B7}, and R^{B8} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R⁸ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{B3} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, or optionally substituted 5-10 membered heteroaryl;
R^{B4} is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
R^{B9} and R^{B10} are independently hydrogen or optionally substituted C₁₋₆ alkyl, or optionally R^{B9} and R^{B10} are joined together with the intervening atoms to form optionally substituted C₃₋₈ carbocyclyl or optionally substituted 3-8 membered heterocyclyl;
each instance of R^{B11} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
m and n are each independently 0, 1, 2, or 3;
q is 0, 1, 2, 3, 4, or 5; and
v2 is 0, 1, 2, 3, or 4.

9. The compound of claim 8, wherein the compound is of Formula (B-II): 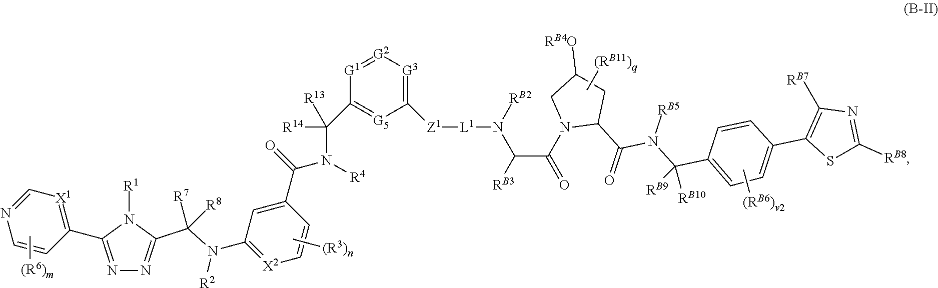
or a pharmaceutically acceptable salt, stereoisomer, tautomer, or isotopically labeled derivative thereof.

10. The compound of claim 8 or 9, wherein the compound is of Formula (B-III): 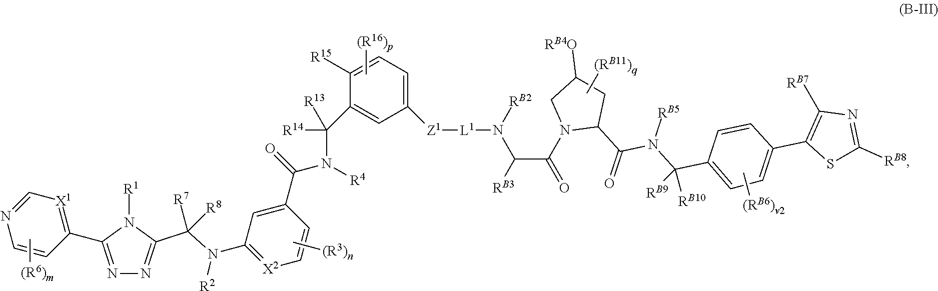
or a pharmaceutically acceptable salt, stereoisomer, or tautomer, or isotopically labeled derivative thereof,
wherein p is 0, 1, 2, or 3.

11. The compound of any one of claims 8-10, wherein the compound is of Formula (B-IV): 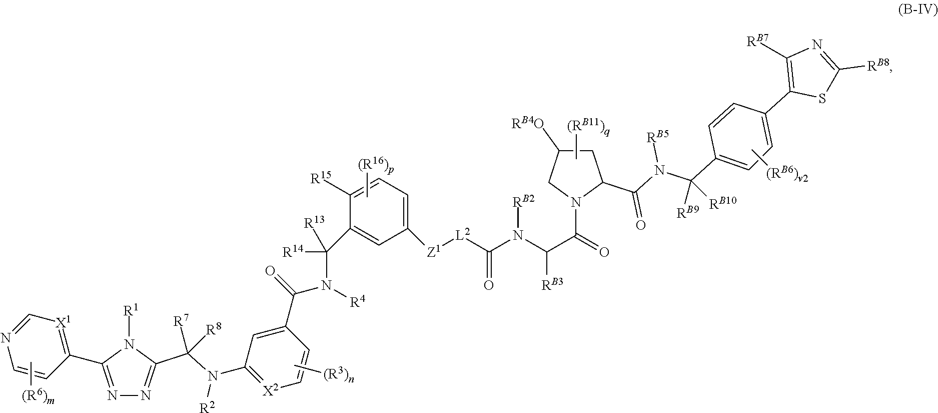
or a pharmaceutically acceptable salt, stereoisomer, or tautomer, or isotopically labeled derivative thereof,
wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

12. The compound of claim 8, wherein the compound is of Formula (B-V): 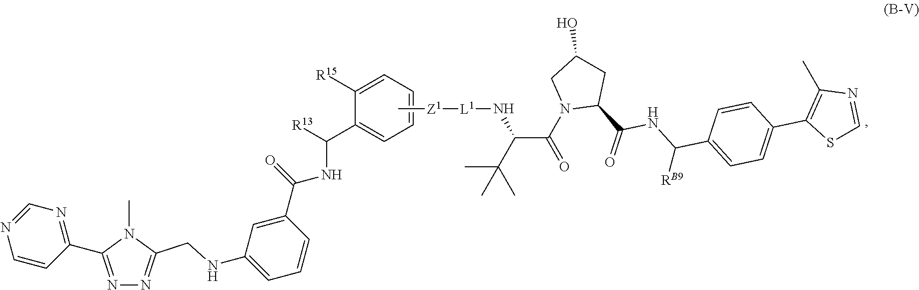
or a pharmaceutically acceptable salt, stereoisomer, or tautomer, or isotopically labeled derivative thereof.

13. The compound of claim 10, wherein the compound is of Formula (B-VI): 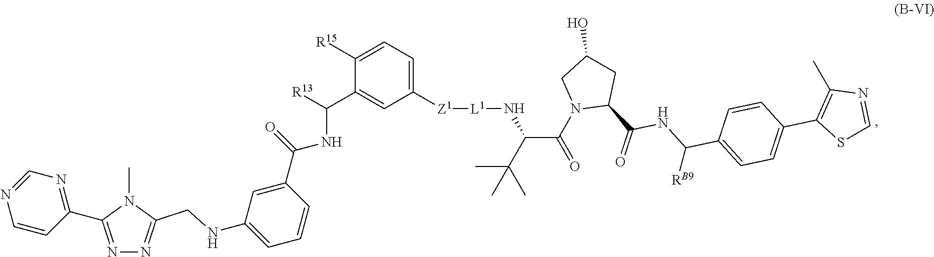
or a pharmaceutically acceptable salt, stereoisomer, or tautomer, or isotopically labeled derivative thereof.

14. The compound of claim 11, wherein the compound is of Formula (B-VII): 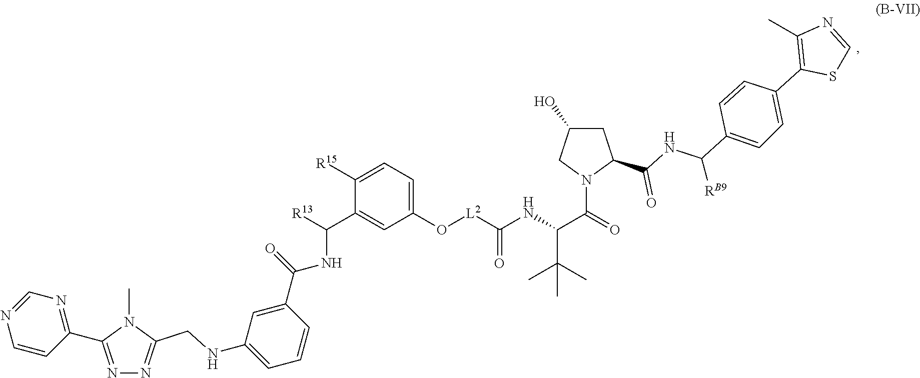
or a pharmaceutically acceptable salt, stereoisomer, or tautomer, or isotopically labeled derivative thereof.

15. A compound of Formula (C-I): 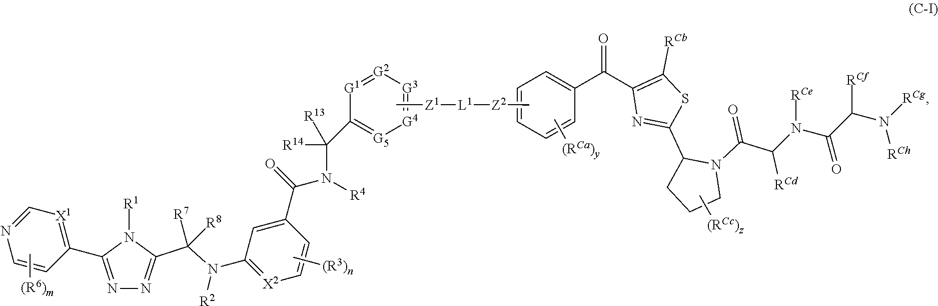
or a pharmaceutically acceptable salt, stereoisomer, tautomer, or isotopically labeled derivative thereof,
wherein:
L¹ is a linker selected from the group consisting of optionally substituted C₁₋₄₀ alkylene, optionally substituted C₂₋₄₀ alkenylene, optionally substituted C₂₋₄₀ alkynylene, optionally substituted C₁₋₄₀ heteroalkylene, optionally substituted C₂₋₄₀ heteroalkenylene, optionally substituted C₂₋₄₀ heteroalkynylene, or optionally substituted C₁₋₄₀ acylene, optionally interrupted with one or more instances of optionally substituted C₃₋₈ carbocyclylene, optionally substituted 3-8 membered heterocyclylene, optionally substituted C₆₋₁₀ arylene, optionally substituted 5-10 membered heteroarylene, and any combination thereof;
Z¹ and Z² are each independently a bond, —O—, —NR^{N}—, —S—, —C(═O)—, or optionally substituted —CH₂—;
G¹ is CR¹⁵ or N;
G², G³, G⁴, and G⁵ are each independently CR¹⁶, CH, or N;
X¹ is CR⁹ or N;
X² is CR³ or N;
R¹ is hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group;
R², R⁴, R^{Ce}, R^{Cg}, and R^{Ch} are each independently hydrogen, optionally substituted C₁₋₆ alkyl, optionally substituted C₁₋₆ acyl, or a nitrogen protecting group;
optionally wherein R^{Cg} and R^{Ch} are joined together with the intervening atoms to form optionally substituted optionally substituted 3-8 membered heterocyclyl or optionally substituted 5-10 membered heteroaryl;
each instance of R³, R⁶, R⁹, R¹⁵, R¹⁶, R^{Ca}, and R^{Cb} is independently hydrogen, halogen, —CN, —N₃, —NO₂, optionally substituted C₁₋₆ alkyl, optionally substituted C₁₋₆ heteroalkyl, optionally substituted C₂₋₆ alkenyl, optionally substituted C₂₋₆ alkynyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, —OR^{O}, —N(R^{N})₂, or —SR^{S};
optionally wherein R⁹ and R¹ are joined together with the intervening atoms to form optionally substituted 4-8 membered heterocyclyl;
R⁷ and R⁸ are independently hydrogen, deuterium, or optionally substituted C₁₋₆ alkyl;
R¹³ and R¹⁴ are independently hydrogen or optionally substituted C₁₋₆ alkyl, or optionally R¹³ and R¹⁴ are joined together with the intervening atoms to form optionally substituted C₃₋₈ carbocyclyl or 3-8 membered heterocyclyl;
optionally wherein R¹³ and R¹⁵ are joined together with the intervening atoms to form optionally substituted C₄₋₈ carbocyclyl or optionally substituted 4-8 membered heterocyclyl;
R^{Cd} and R^{Cf} are each independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, or optionally substituted C₁ 6 acyl;
each instance of R^{cc} is independently halogen or optionally substituted C₁-C₆ alkyl;
each instance of R^{O} is independently hydrogen, optionally substituted C₁₋₆ alkyl, optionally substituted C₁₋₆ heteroalkyl, optionally substituted C₃₋₁₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or an oxygen protecting group;
each instance of R^{N} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a nitrogen protecting group, or optionally two R^{N} bonded to the same nitrogen atom are taken together with the intervening atoms to form optionally substituted optionally substituted 3-8 membered heterocyclyl or optionally substituted 5-10 membered heteroaryl;
each instance of R^{S} is independently hydrogen, optionally substituted C₁₋₆ alkyl, optionally substituted C₁₋₆ heteroalkyl, optionally substituted C₃₋₈ carbocyclyl, optionally substituted 3-8 membered heterocyclyl, optionally substituted C₆₋₁₀ aryl, optionally substituted 5-10 membered heteroaryl, optionally substituted C₁₋₆ acyl, or a sulfur protecting group;
y is 0, 1, 2, 3, or 4; and
z is 0, 1, 2, 3, 4, 5, 6, or 7.

16. The compound of claim 15, wherein the compound is of Formula (C-II): 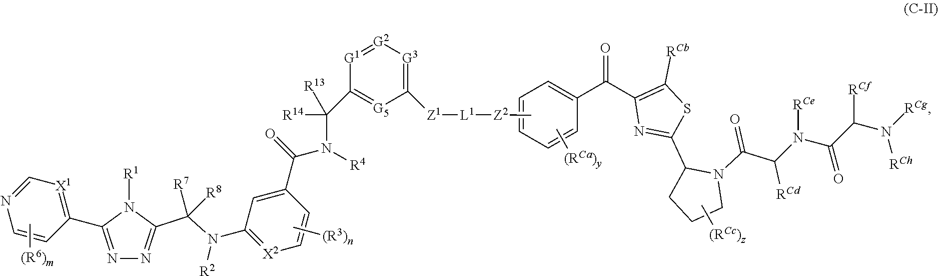
or a pharmaceutically acceptable salt, stereoisomer, tautomer, or isotopically labeled derivative thereof.

17. The compound of claim 15 or 16, wherein the compound is of Formula (C-III): 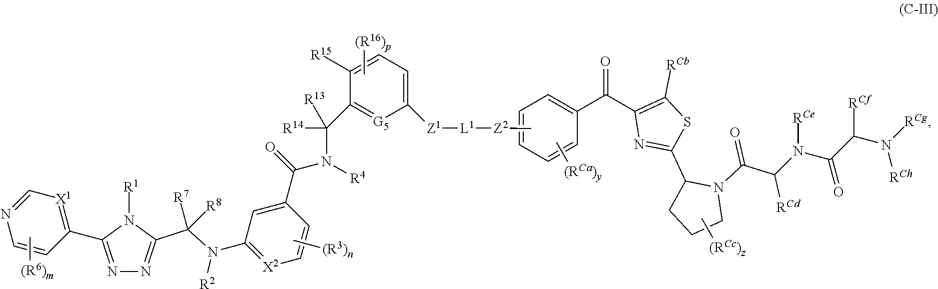
or a pharmaceutically acceptable salt, stereoisomer, or tautomer, or isotopically labeled derivative thereof, wherein p is 0, 1, 2, or 3.

18. The compound of any one of claims 15-17, wherein the compound is of Formula (C-IV): 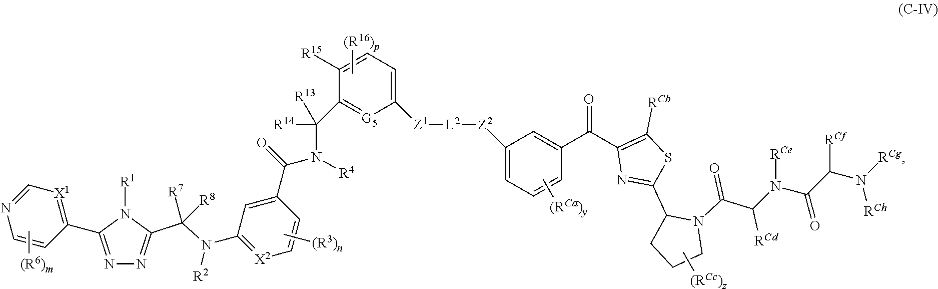
or a pharmaceutically acceptable salt, stereoisomer, or tautomer, or isotopically labeled derivative thereof,
wherein:
L² is a linker selected from the group consisting of optionally substituted C₁-C₃₀ alkylene, optionally substituted C₂-C₃₀ alkenylene, optionally substituted C₂-C₃₀ alkynylene, optionally substituted C₁-C₃₀ heteroalkylene, optionally substituted C₂-C₃₀ heteroalkenylene, optionally substituted C₂-C₃₀ heteroalkynylene, or optionally substituted C₁₋₃₀ acylene, optionally interrupted with one or more instances of optionally substituted C₃-C₈ carbocyclylene, optionally substituted 3-8 membered heterocyclylene, optionally substituted C₆-C₁₀ arylene, or optionally substituted 5-10 membered heteroarylene.

19. The compound of claim 15, wherein the compound is of Formula (C-V): 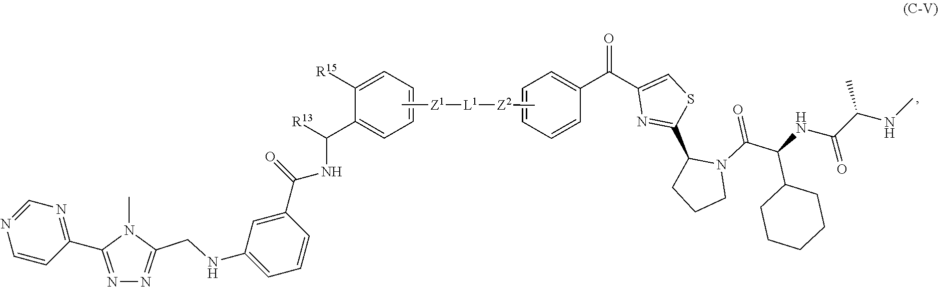
or a pharmaceutically acceptable salt, stereoisomer, or tautomer, or isotopically labeled derivative thereof.

20. The compound of claim 17, wherein the compound is of Formula (C-VI): 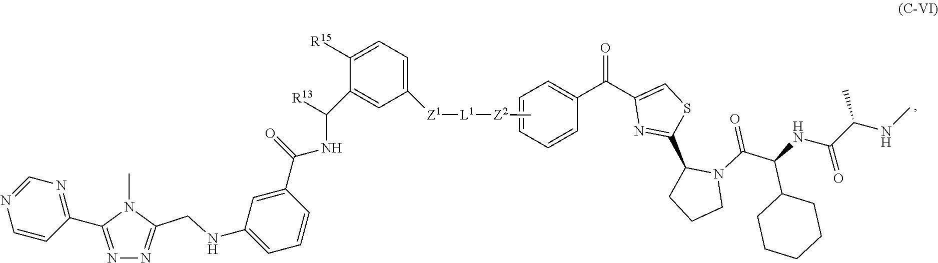
or a pharmaceutically acceptable salt, stereoisomer, or tautomer, or isotopically labeled derivative thereof.

21. The compound of claim 18, wherein the compound is of Formula (C-VII): 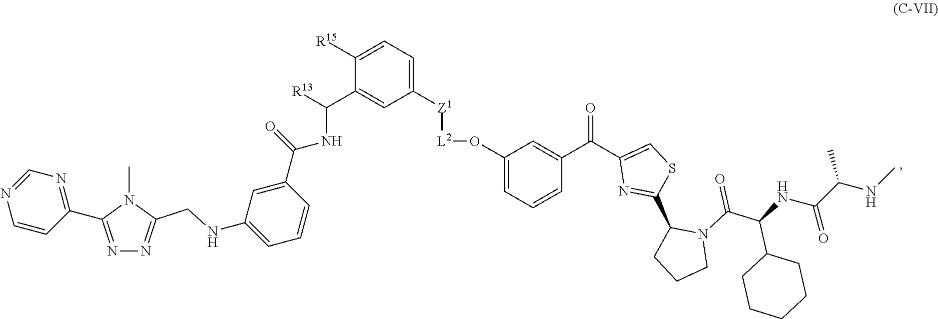
or a pharmaceutically acceptable salt, stereoisomer, or tautomer, or isotopically labeled derivative thereof.

22. The compound of any one of the preceding claims, or a pharmaceutically acceptable salt, stereoisomer, or tautomer, or isotopically labeled derivative thereof, wherein X¹ is N.

23. The compound of any one of the preceding claims, or a pharmaceutically acceptable salt, stereoisomer, or tautomer, or isotopically labeled derivative thereof, wherein X² is CR³.

24. The compound of any one of the preceding claims, or a pharmaceutically acceptable salt, stereoisomer, or tautomer, or isotopically labeled derivative thereof, wherein R¹ is C₁₋₆ alkyl.

25. The compound of claim 24, or a pharmaceutically acceptable salt, stereoisomer, or tautomer, or isotopically labeled derivative thereof, wherein R¹ is methyl.

26. The compound of any one of the preceding claims, or a pharmaceutically acceptable salt, stereoisomer, or tautomer, or isotopically labeled derivative thereof, wherein R² is hydrogen.

27. The compound of any one of the preceding claims, or a pharmaceutically acceptable salt, stereoisomer, or tautomer, or isotopically labeled derivative thereof, wherein R⁴ is hydrogen.

28. The compound of any one of the preceding claims, or a pharmaceutically acceptable salt, stereoisomer, or tautomer, or isotopically labeled derivative thereof, wherein each instance of R³ is hydrogen.

29. The compound of any one of the preceding claims, or a pharmaceutically acceptable salt, stereoisomer, or tautomer, or isotopically labeled derivative thereof, wherein m is 0.

30. The compound of any one of the preceding claims, or a pharmaceutically acceptable salt, stereoisomer, or tautomer, or isotopically labeled derivative thereof, wherein n is 0.

31. The compound of any one of the preceding claims, or a pharmaceutically acceptable salt, stereoisomer, or tautomer, or isotopically labeled derivative thereof, wherein R⁷ and R⁸ are hydrogen.

32. The compound of any one of the preceding claims, or a pharmaceutically acceptable salt, stereoisomer, or tautomer, or isotopically labeled derivative thereof, wherein R¹³ is hydrogen.

33. The compound of any one of the preceding claims, or a pharmaceutically acceptable salt, stereoisomer, or tautomer, or isotopically labeled derivative thereof, wherein R¹³ is C₁₋₆ alkyl.

34. The compound of claim 33, or a pharmaceutically acceptable salt, stereoisomer, or tautomer, or isotopically labeled derivative thereof, wherein R¹³ is methyl.

35. The compound of any one of the preceding claims, or a pharmaceutically acceptable salt, stereoisomer, or tautomer, or isotopically labeled derivative thereof, wherein R¹⁴ is hydrogen.

36. The compound of any one of the preceding claims, or a pharmaceutically acceptable salt, stereoisomer, or tautomer, or isotopically labeled derivative thereof, wherein G¹ is CR¹⁵.

37. The compound of any one of the preceding claims, or a pharmaceutically acceptable salt, stereoisomer, or tautomer, or isotopically labeled derivative thereof, wherein G², G³, G⁴, and G⁵ are independently CR¹⁶ or CH.

38. The compound of any one of the preceding claims, or a pharmaceutically acceptable salt, stereoisomer, or tautomer, or isotopically labeled derivative thereof, wherein R¹⁵ is selected from halogen, C₁₋₆ haloalkyl, and —O—C₁₋₆ alkyl.

39. The compound of claim 38, or a pharmaceutically acceptable salt, stereoisomer, or tautomer, or isotopically labeled derivative thereof, wherein R¹⁵ is selected from —F, —CF₃, and —OMe.

40. The compound of any one of the preceding claims, wherein R¹³ and R¹⁵ are joined together to form:

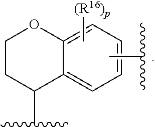

41. The compound of any one of the preceding claims, or a pharmaceutically acceptable salt, stereoisomer, or tautomer, or isotopically labeled derivative thereof, wherein p is 0.

42. The compound of any one of the preceding claims, or a pharmaceutically acceptable salt, stereoisomer, or tautomer, or isotopically labeled derivative thereof, wherein Q¹, Q², Q³, and Q⁴ are independently CR^{A1} or CH.

43. The compound of any one of the preceding claims, or a pharmaceutically acceptable salt, stereoisomer, or tautomer, or isotopically labeled derivative thereof, wherein s is 0.

44. The compound of any one of the preceding claims, or a pharmaceutically acceptable salt, stereoisomer, or tautomer, or isotopically labeled derivative thereof, wherein R^{A5} is hydrogen.

45. The compound of any one of the preceding claims, or a pharmaceutically acceptable salt, stereoisomer, or tautomer, or isotopically labeled derivative thereof, wherein R^{A6} and R^{A7} are hydrogen.

46. The compound of any one of the preceding claims, or a pharmaceutically acceptable salt, stereoisomer, or tautomer, or isotopically labeled derivative thereof, wherein r is 0.

47. The compound of any one of the preceding claims, or a pharmaceutically acceptable salt, stereoisomer, or tautomer, or isotopically labeled derivative thereof, wherein R^{B2} is hydrogen.

48. The compound of any one of the preceding claims, or a pharmaceutically acceptable salt, stereoisomer, or tautomer, or isotopically labeled derivative thereof, wherein R^{B3} C₁₋₆ alkyl.

49. The compound of claim 48, or a pharmaceutically acceptable salt, stereoisomer, or tautomer, or isotopically labeled derivative thereof, wherein R^{3B} is tert-butyl.

50. The compound of any one of the preceding claims, or a pharmaceutically acceptable salt, stereoisomer, or tautomer, or isotopically labeled derivative thereof, wherein R^{B4} is hydrogen

51. The compound of any one of the preceding claims, or a pharmaceutically acceptable salt, stereoisomer, or tautomer, or isotopically labeled derivative thereof, wherein R^{B5} is hydrogen

52. The compound of any one of the preceding claims, or a pharmaceutically acceptable salt, stereoisomer, or tautomer, or isotopically labeled derivative thereof, wherein R^{B7} is C₁₋₆ alkyl.

53. The compound of claim 52, or a pharmaceutically acceptable salt, stereoisomer, or tautomer, or isotopically labeled derivative thereof, wherein R^{B7} is methyl.

54. The compound of any one of the preceding claims, or a pharmaceutically acceptable salt, stereoisomer, or tautomer, or isotopically labeled derivative thereof, wherein R^{B8} is hydrogen.

55. The compound of any one of the preceding claims, or a pharmaceutically acceptable salt, stereoisomer, or tautomer, or isotopically labeled derivative thereof, wherein R^{B9} is hydrogen.

56. The compound of any one of the preceding claims, or a pharmaceutically acceptable salt, stereoisomer, or tautomer, or isotopically labeled derivative thereof, wherein R^{B9} is C₁₋₆ alkyl.

57. The compound of claim 56, or a pharmaceutically acceptable salt, stereoisomer, or tautomer, or isotopically labeled derivative thereof, wherein R^{B9} is methyl.

58. The compound of any one of the preceding claims, or a pharmaceutically acceptable salt, stereoisomer, or tautomer, or isotopically labeled derivative thereof, wherein R^{B10} is hydrogen.

59. The compound of any one of the preceding claims, or a pharmaceutically acceptable salt, stereoisomer, or tautomer, or isotopically labeled derivative thereof, wherein q is 0.

60. The compound of any one of the preceding claims, or a pharmaceutically acceptable salt, stereoisomer, or tautomer, or isotopically labeled derivative thereof, wherein v2 is 0.

61. The compound of any one of the preceding claims, or a pharmaceutically acceptable salt, stereoisomer, or tautomer, or isotopically labeled derivative thereof, wherein y is 0.

62. The compound of any one of the preceding claims, or a pharmaceutically acceptable salt, stereoisomer, or tautomer, or isotopically labeled derivative thereof, wherein z is 0.

63. The compound of any one of the preceding claims, or a pharmaceutically acceptable salt, stereoisomer, or tautomer, or isotopically labeled derivative thereof, wherein R^{Cb} is hydrogen.

64. The compound of any one of the preceding claims, or a pharmaceutically acceptable salt, stereoisomer, or tautomer, or isotopically labeled derivative thereof, wherein R^{Cd} is C₃₋₈ carbocyclyl.

65. The compound of claim 64, or a pharmaceutically acceptable salt, stereoisomer, or tautomer, or isotopically labeled derivative thereof, wherein R^{Cd} is:

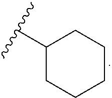 [SMILES: *C1CCCCC1]

66. The compound of any one of the preceding claims, or a pharmaceutically acceptable salt, stereoisomer, or tautomer, or isotopically labeled derivative thereof, wherein R^{Ce} is hydrogen.

67. The compound of any one of the preceding claims, or a pharmaceutically acceptable salt, stereoisomer, or tautomer, or isotopically labeled derivative thereof, wherein R^{Cf} is C₁₋₆ alkyl.

68. The compound of claim 67, or a pharmaceutically acceptable salt, stereoisomer, or tautomer, or isotopically labeled derivative thereof, wherein R^{Cf} is methyl.

69. The compound of any one of the preceding claims, or a pharmaceutically acceptable salt, stereoisomer, or tautomer, or isotopically labeled derivative thereof, wherein R^{Cg} is C₁₋₆ alkyl.

70. The compound of claim 69, or a pharmaceutically acceptable salt, stereoisomer, or tautomer, or isotopically labeled derivative thereof, wherein R^{Cg} is methyl.

71. The compound of any one of the preceding claims, or a pharmaceutically acceptable salt, stereoisomer, or tautomer, or isotopically labeled derivative thereof, wherein R^{Ch} is hydrogen.

72. The compound of any one of the preceding claims, or a pharmaceutically acceptable salt, stereoisomer, or tautomer, or isotopically labeled derivative thereof, wherein Z¹ is —O—.

73. The compound of any one of the preceding claims, or a pharmaceutically acceptable salt, stereoisomer, or tautomer, or isotopically labeled derivative thereof, wherein Z² is —NR^{A11}— or —NR^{N}—.

74. The compound of any one of the preceding claims, or a pharmaceutically acceptable salt, stereoisomer, or tautomer, or isotopically labeled derivative thereof, wherein R^{A11} is hydrogen.

75. The compound of any one of the preceding claims, or a pharmaceutically acceptable salt, stereoisomer, or tautomer, or isotopically labeled derivative thereof, wherein Z² is —O—.

76. The compound of any one of the preceding claims, or a pharmaceutically acceptable salt, stereoisomer, or tautomer, or isotopically labeled derivative thereof, wherein L¹ is optionally substituted C₁₋₄₀ alkylene or optionally substituted C₁₋₄₀ heteroalkylene.

77. The compound of claim 76, or a pharmaceutically acceptable salt, stereoisomer, or tautomer, or isotopically labeled derivative thereof, wherein L¹ is comprises one or more groups independently selected from —C(═O)—, —O—, —NR^{N}—, —OC(═O)—, —C(═O)O—, —NR^{N}C(═O)—, and —C(═O)NR^{N}—; and/or L¹ is interrupted with at least one instance of optionally substituted 4-6 membered heterocyclylene comprising 1 or 2 heteroatoms selected from N and O.

78. The compound of any one of the preceding claims, or a pharmaceutically acceptable salt, stereoisomer, or tautomer, or isotopically labeled derivative thereof, wherein L¹ is optionally substituted C₁₋₄₀ alkylene or optionally substituted C₁₋₄₀ heteroalkylene, interrupted with at least one instance of optionally substituted triazolylene.

79. The compound of any one of the preceding claims, or a pharmaceutically acceptable salt, stereoisomer, or tautomer, or isotopically labeled derivative thereof, wherein L¹ is of one of the following formulae:

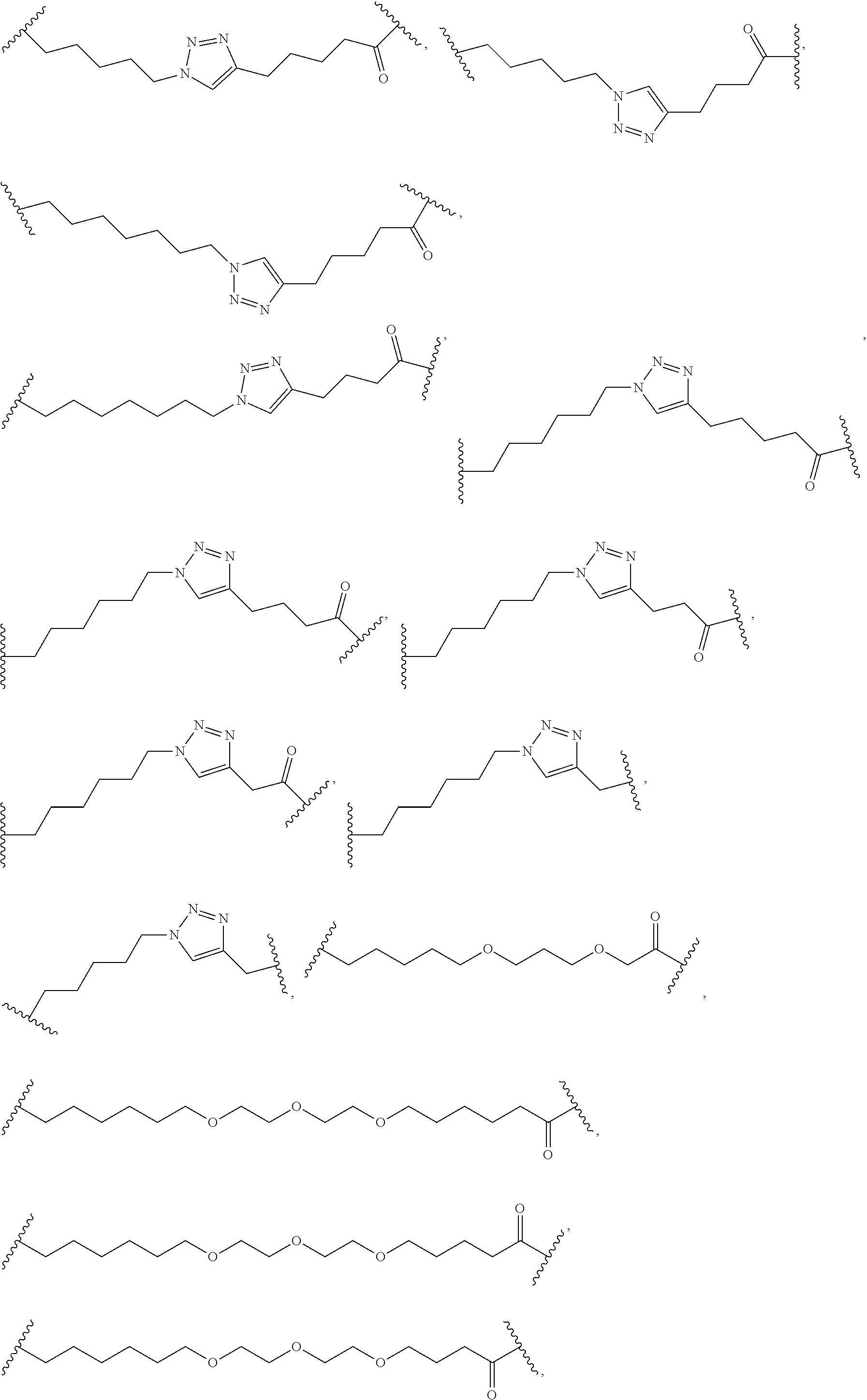

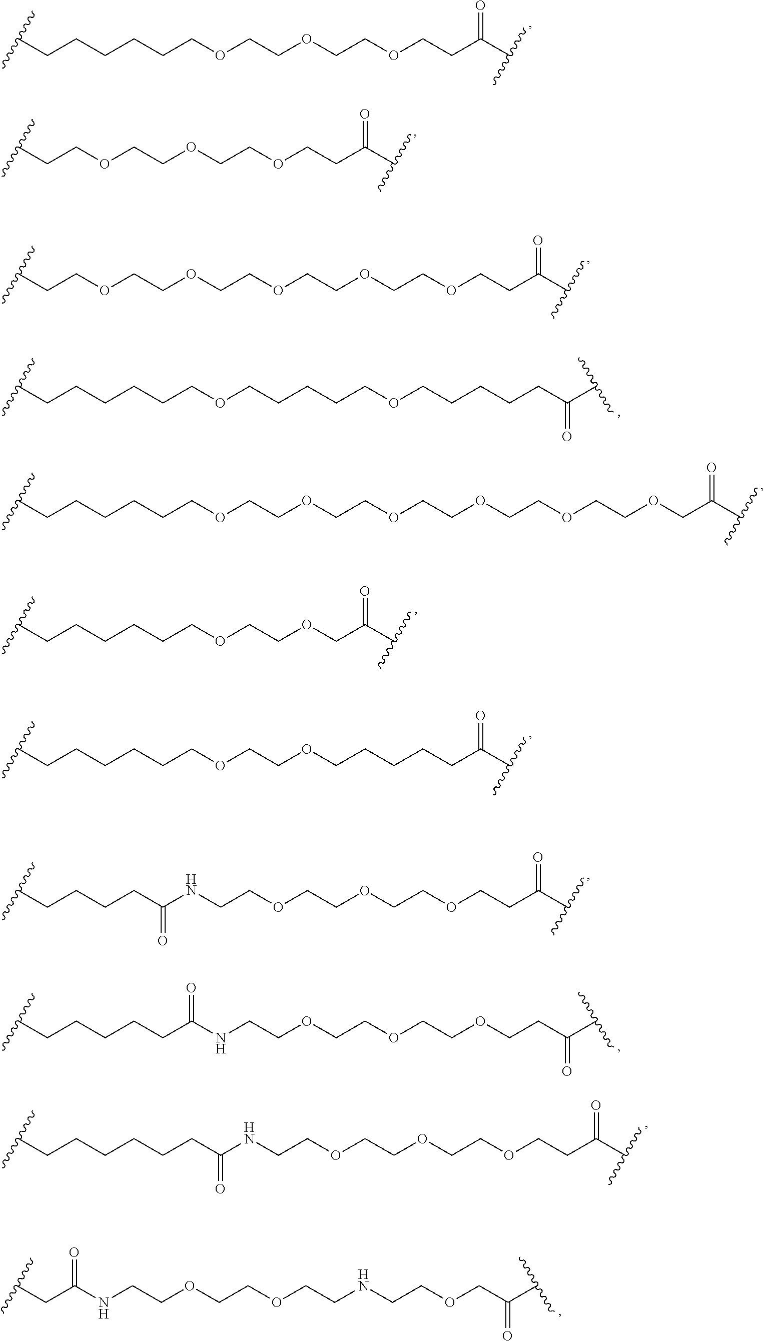

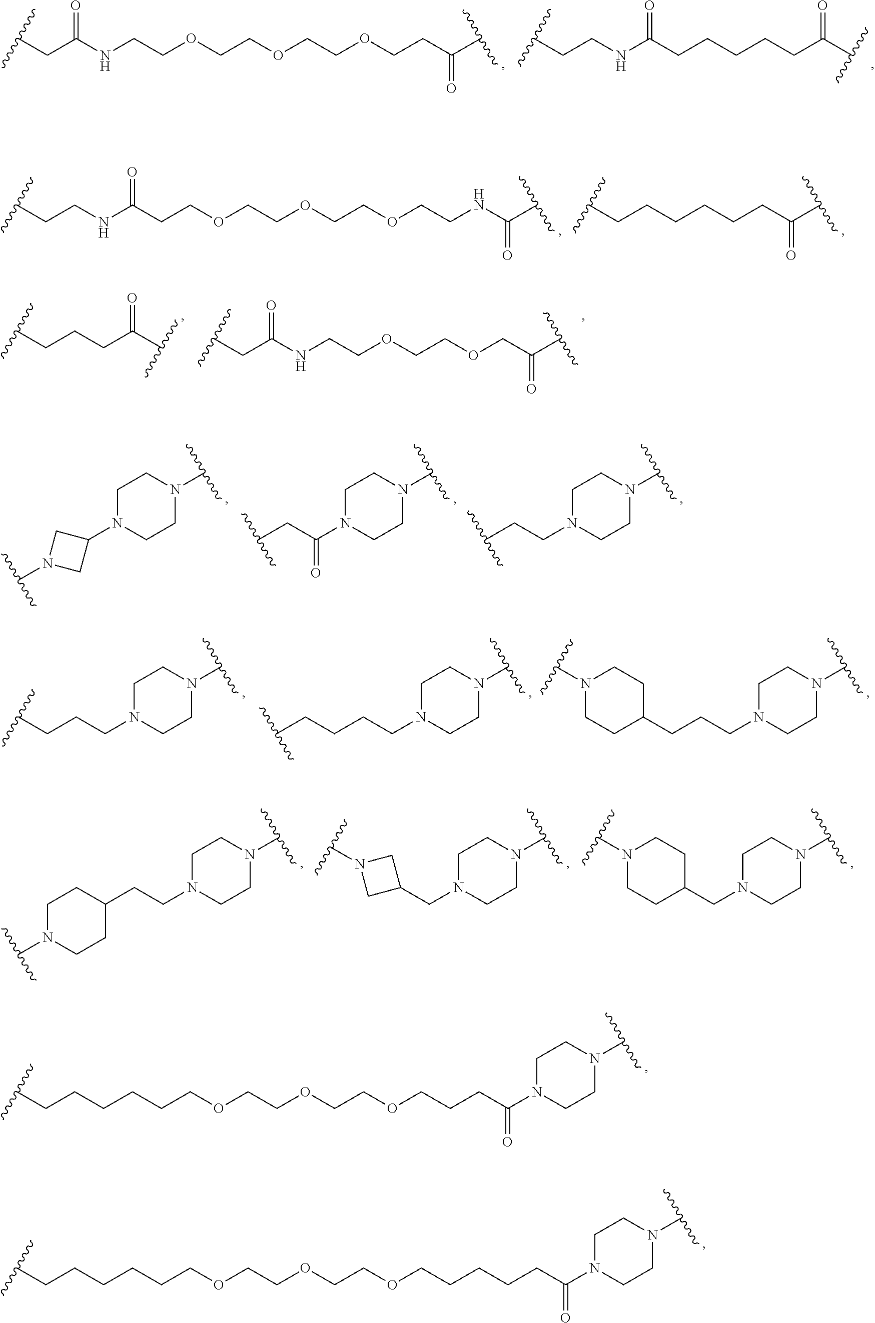

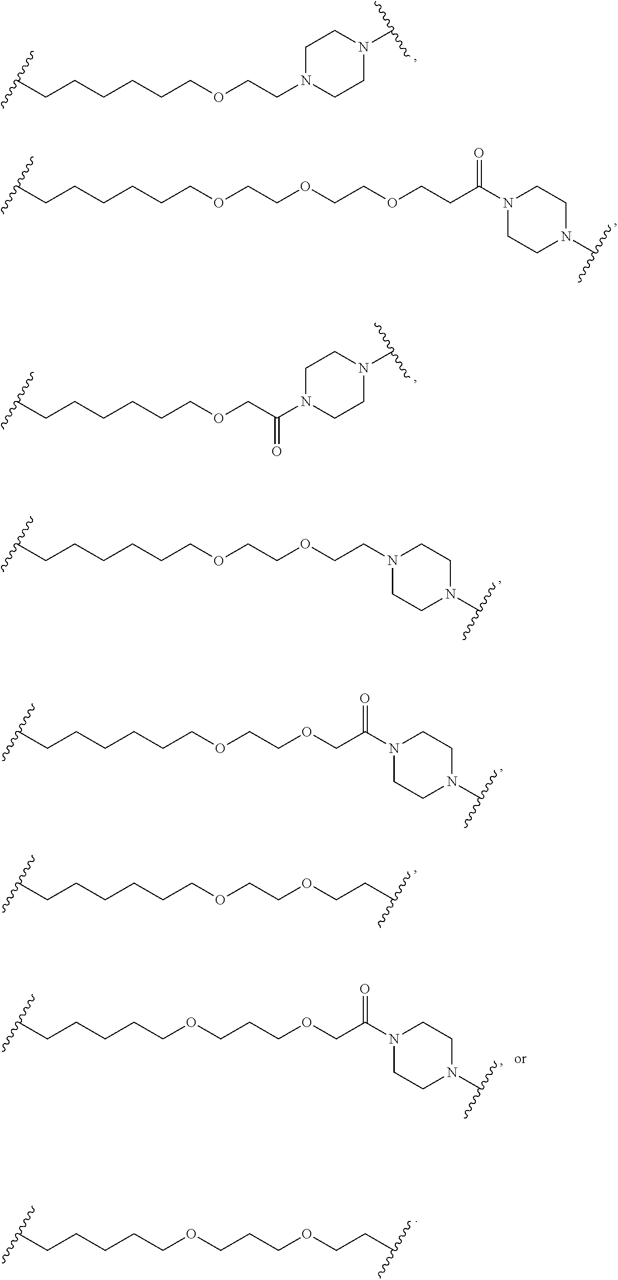

80. The compound of any one of the preceding claims, or a pharmaceutically acceptable salt, stereoisomer, or tautomer, or isotopically labeled derivative thereof, wherein L² is optionally substituted C₁-C₃₀ alkylene or optionally substituted C₁-C₃₀ heteroalkylene.

81. The compound of claim 80, or a pharmaceutically acceptable salt, stereoisomer, or tautomer, or isotopically labeled derivative thereof, wherein L² is comprises one or more groups independently selected from —C(═O)—, —O—, —NR^{N}—, —OC(═O)—, —C(═O)O—, —NR^{N}C(═O)—, and —C(═O)NR^{N}—.

82. The compound of any one of the preceding claims, or a pharmaceutically acceptable salt, stereoisomer, or tautomer, or isotopically labeled derivative thereof, wherein L² is optionally substituted C₁-C₃₀ alkylene or optionally substituted C₁-C₃₀ heteroalkylene, interrupted with at least one instance of optionally substituted triazolylene.

83. The compound of any one of the preceding claims, or a pharmaceutically acceptable salt, stereoisomer, or tautomer, or isotopically labeled derivative thereof, wherein L² is of one of the following formulae:

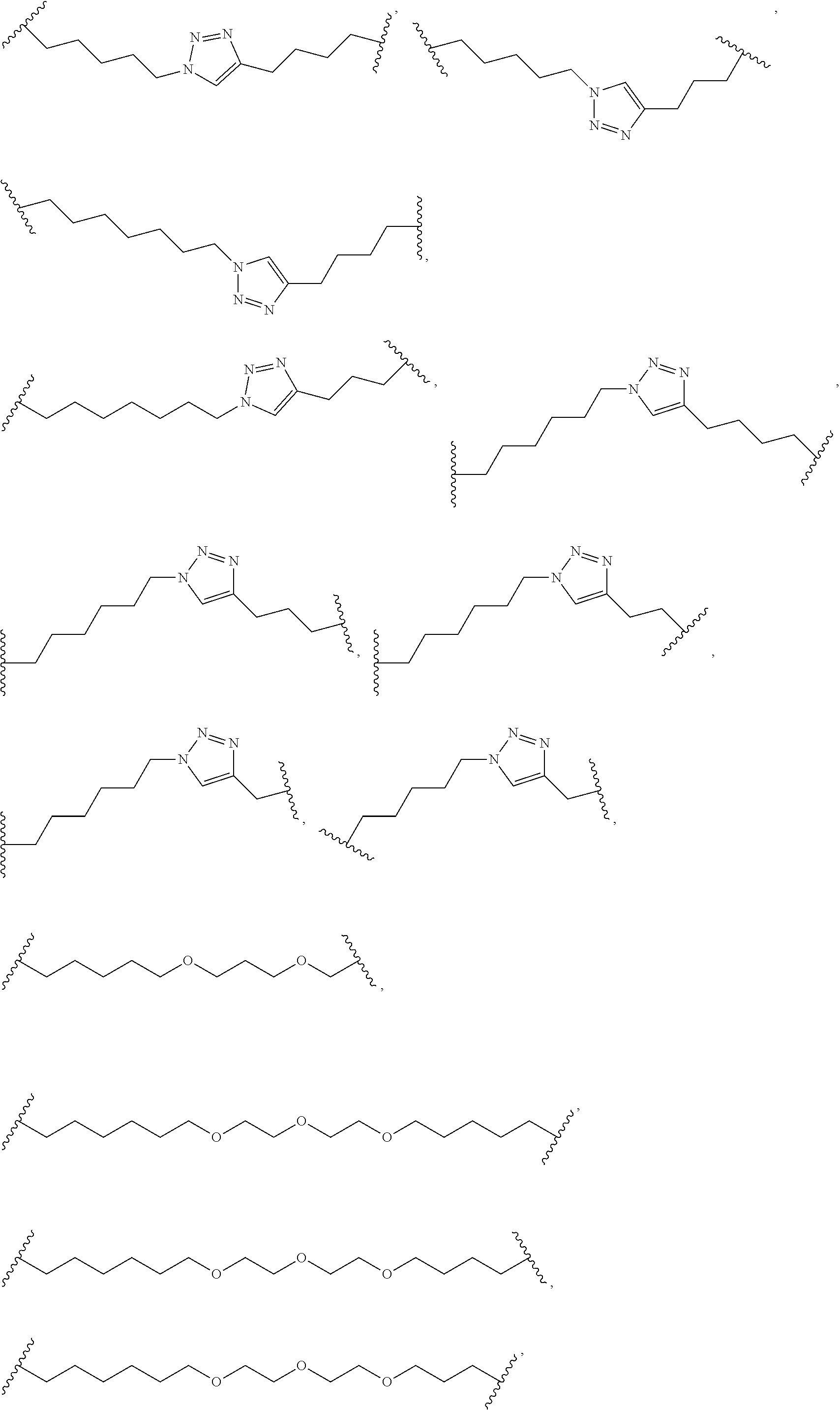

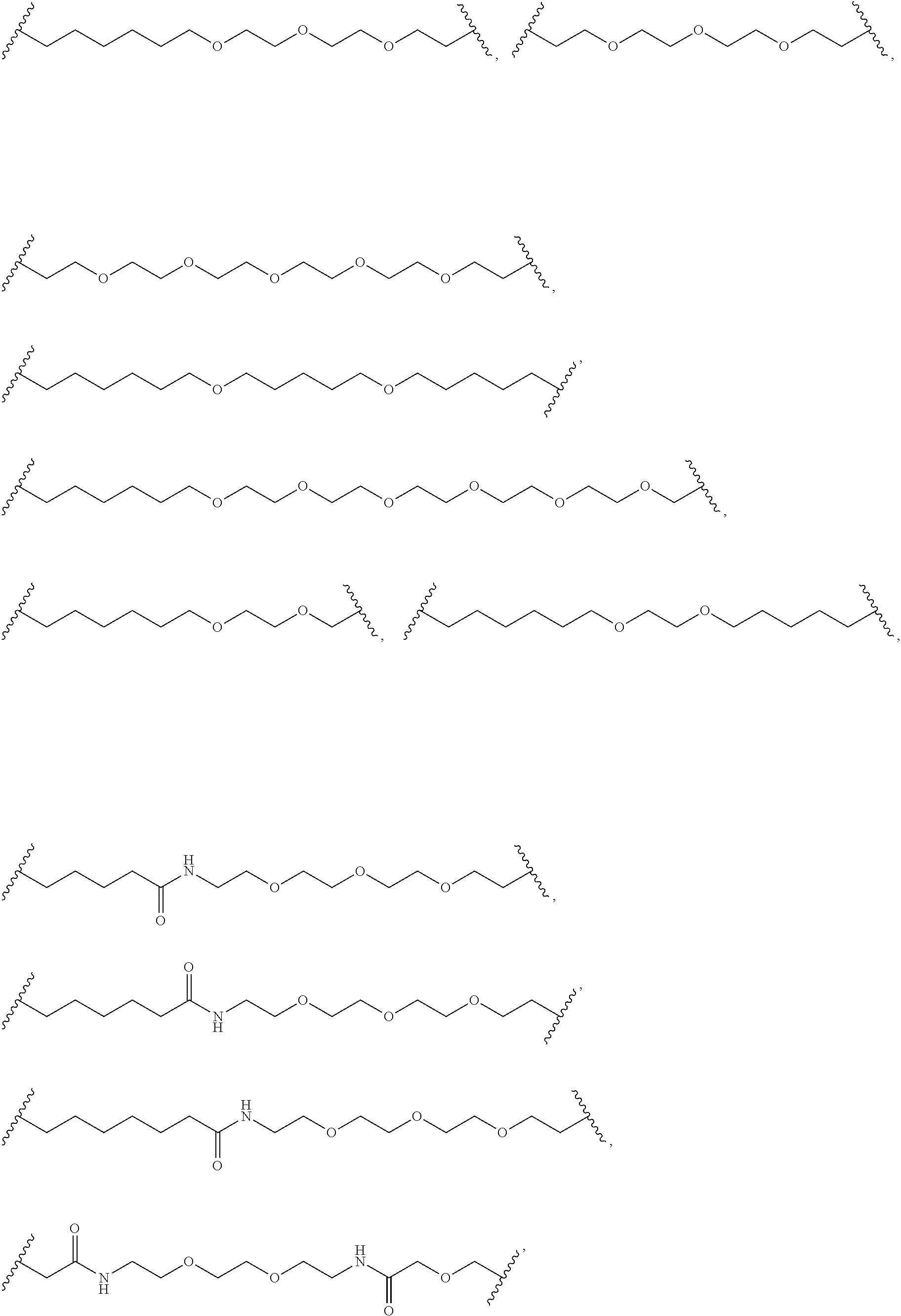

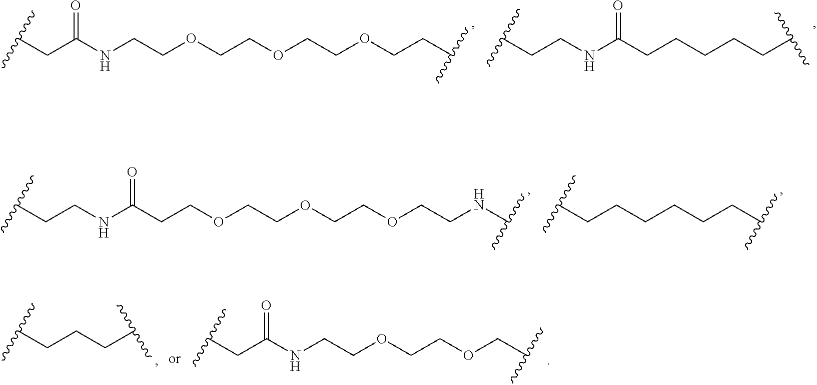

84. The compound of any one of the preceding claims, or a pharmaceutically acceptable salt, stereoisomer, or tautomer, or isotopically labeled derivative thereof, wherein at least one instance of R^{N} is hydrogen.

85. The compound of claim 1, wherein the compound is selected from those in Table A, and pharmaceutically acceptable salts and tautomers thereof.

86. The compound of claim 8, wherein the compound is selected from those in Table B, and pharmaceutically acceptable salts and tautomers thereof.

87. The compound of claim 15, wherein the compound is selected from those in Table C, and pharmaceutically acceptable salts and tautomers thereof.

88. A pharmaceutical composition comprising a compound of any one of the preceding claims, or a pharmaceutically acceptable salt, stereoisomer, or tautomer, or isotopically labeled derivative thereof, and a pharmaceutically acceptable excipient.

89. A method of treating a proliferative disease in a subject comprising administering to the subject a compound of any one of the preceding claims, or a pharmaceutically acceptable salt, stereoisomer, or tautomer, or isotopically labeled derivative thereof, or a pharmaceutical composition thereof.

90. The method of claim 89, wherein the proliferative disease is cancer.

91. The method of claim 90, wherein the cancer is pancreatic cancer.

92. A method of inhibiting tumor growth in a subject comprising administering to the subject a compound of any one of the preceding claims, or a pharmaceutically acceptable salt, stereoisomer, or tautomer, or isotopically labeled derivative thereof, or a pharmaceutical composition thereof.

93. A method of treating a cardiovascular disease in a subject comprising administering to the subject a compound of any one of the preceding claims, or a pharmaceutically acceptable salt, stereoisomer, or tautomer, or isotopically labeled derivative thereof, or a pharmaceutical composition thereof.

94. The method of claim 93, wherein the cardiovascular disease is heart failure, cardiac hypertrophy, or hypertension.

95. A method of treating a GRK2-related disease in a subject comprising administering to the subject a compound of any one of the preceding claims, or a pharmaceutically acceptable salt, stereoisomer, or tautomer, or isotopically labeled derivative thereof, or a pharmaceutical composition thereof.

96. A method of inhibiting the activity of GRK2 in a subject or biological sample comprising administering to the subject or contacting the biological sample with a compound of any one of the preceding claims, or a pharmaceutically acceptable salt, stereoisomer, or tautomer, or isotopically labeled derivative thereof, or a pharmaceutical composition thereof.

97. A method of degrading a GRK2 protein in a subject or biological sample comprising administering to the subject or contacting the biological sample with a compound of any one of the preceding claims, or a pharmaceutically acceptable salt, stereoisomer, or tautomer, or isotopically labeled derivative thereof, or a pharmaceutical composition thereof.

98. The method of any one of the preceding claims, wherein the subject is a human.

99. A compound of any one of the preceding claims, or a pharmaceutically acceptable salt, stereoisomer, or tautomer, or isotopically labeled derivative thereof, or a pharmaceutical composition thereof, for use in a method of any one of the preceding claims.

100. Use of a compound of any one of the preceding claims, or a pharmaceutically acceptable salt, stereoisomer, or tautomer, or isotopically labeled derivative thereof, or a pharmaceutical composition thereof, for the manufacture of a medicament.

101. A kit comprising a compound of any one of the preceding claims, or a pharmaceutically acceptable salt, stereoisomer, or tautomer, or isotopically labeled derivative thereof, or a pharmaceutical composition thereof; and optionally instructions for use.


SEQUENCE LISTING (3 sequences)

SEQ ID NO: 1 - DNA, 2070 bp, Homo sapiens
atggcggacctggaggcggtgctggccgacgtgagctacctgatggccatggagaagagcaaggccacgccggccgcgcgcgccagcaagaagatcctgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaaggctgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacgctccctgctggaggggttgctgcagagggatgtcaaccggagattgggctgcctgggccgaggggctcaggaggtgaaagagagcccctttttccgctccctggactggcagatggtcttcttgcagaagtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaaccccttcctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctga

SEQ ID NO: 2 - protein, 689 aa, Homo sapiens
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKADTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELRSLLEGLLQRDVNRRLGCLGRGAQEVKESPFFRSLDWQMVFLQKYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL

SEQ ID NO: 3 - protein, 688 aa, Homo sapiens
MADLEAVLADVSYLMAMEKSKATPAARASKRIVLPEPSIRSVMQKYLAERNEITFDKIFNQKIGFLLFKDFCLNEINEAVPQVKFYEEIKEYEKLDNEEDRLCRSRQIYDAYIMKELLSCSHPFSKQAVEHVQSHLSKKQVTSTLFQPYIEEICESLRGDIFQKFMESDKFTRFCQWKNVELNIHLTMNEFSVHRIIGRGGFGEVYGCRKADTGKMYAMKCLDKKRIKMKQGETLALNERIMLSLVSTGDCPFIVCMTYAFHTPDKLCFILDLMNGGDLHYHLSQHGVFSEKEMRFYATEIILGLEHMHNRFVVYRDLKPANILLDEHGHARISDLGLACDFSKKKPHASVGTHGYMAPEVLQKGTAYDSSADWFSLGCMLFKLLRGHSPFRQHKTKDKHEIDRMTLTVNVELPDTFSPELKSLLEGLLQRDVSKRLGCHGGGSQEVKEHSFFKGVDWQHVYLQKYPPPLIPPRGEVNAADAFDIGSFDEEDTKGIKLLDCDQELYKNFPLVISERWQQEVTETVYEAVNADTDKIEARKRAKNKQLGHEEDYALGKDCIMHGYMLKLGNPFLTQWQRRYFYLFPNRLEWRGEGESRQNLLTMEQILSVEETQIKDKKCILFRIKGGKQFVLQCESDPEFVQWKKELNETFKEAQRLLRRAPKFLNKPRSGTVELPKPSLCHRNSNGL